SEQUENCE LISTING (67 sequences)

SEQ ID NO: 1 - protein, 17 aa, Artificial Sequence
  Chemically synthesized
  misc_feature (1)..(1): N-acetylated
  misc_feature (11)..(11): Xaa =norleucin
  misc_feature (17)..(17): terminus is C(=O)NH2
VKFNKPFVFLXIEQNTK

SEQ ID NO: 2 - protein, 17 aa, Artificial Sequence
  Chemically synthesized
VKFNKPFVFLMIEQNTK

SEQ ID NO: 3 - protein, 20 aa, Artificial Sequence
  Chemically synthesized
TWPKHFDKHTFYSILKLGKH

SEQ ID NO: 4 - protein, 19 aa, Artificial Sequence
  Chemically synthesized
TFFYGGSRGKRNNFKTEEY

SEQ ID NO: 5 - protein, 30 aa, Artificial Sequence
  Chemically synthesized
LRKLRKRLLRDADDLLRKLRKRLLRDADDL

SEQ ID NO: 6 - protein, 20 aa, Artificial Sequence
  Chemically synthesized
TEELRVRLASHLRKLRKRLL

SEQ ID NO: 7 - protein, 12 aa, Artificial Sequence
  Chemically synthesized
EAKIEKHNHYQK

SEQ ID NO: 8 - protein, 22 aa, Artificial Sequence
  Chemically synthesized
EAKIEKHNHYQKQLEIAHEKLR

SEQ ID NO: 9 - protein, 19 aa, Artificial Sequence
  Chemically synthesized
TFFYGGSRGKRNNFKTEEY

SEQ ID NO: 10 - protein, 8 aa, Artificial Sequence
  Chemically synthesized
  misc_feature (1)..(1): D-amino acid
  misc_feature (1)..(8): Cys and Pen form a ring
  misc_feature (3)..(3): Xaa = thiazolidine-4-carboxylic acid
  misc_feature (8)..(8): Xaa = Penicillamine
CMXRLRGX

SEQ ID NO: 11 - protein, 8 aa, Artificial Sequence
  Chemically synthesized
  misc_feature (1)..(8): Cys and Cys form a ring
CMPRLRGC

SEQ ID NO: 12 - protein, 12 aa, Artificial Sequence
  Chemically synthesized
  misc_feature (4)..(9): Cys and Cys form a ring
HLDCMPRGCFRN

SEQ ID NO: 13 - protein, 9 aa, Artificial Sequence
  Chemically synthesized
  misc_feature (1)..(9): Cys and Cys form a ring
CQVKSMPRC

SEQ ID NO: 14 - protein, 9 aa, Artificial Sequence
  Chemically synthesized
  misc_feature (1)..(9): Cys and Cys form a ring
CTTPMPRLC

SEQ ID NO: 15 - protein, 9 aa, Artificial Sequence
  Chemically synthesized
  misc_feature (1)..(9): Cys and Cys form a ring
CKAPQMPRC

SEQ ID NO: 16 - protein, 9 aa, Artificial Sequence
  Chemically synthesized
  misc_feature (1)..(9): Cys and Cys form a ring
CLNPSMPRC

SEQ ID NO: 17 - protein, 9 aa, Artificial Sequence
  Chemically synthesized
CLVSSMPRC

SEQ ID NO: 18 - protein, 9 aa, Artificial Sequence
  Chemically synthesized
  misc_feature (1)..(9): Cys and Cys form a ring
CLQPMPRLC

SEQ ID NO: 19 - protein, 9 aa, Artificial Sequence
  Chemically synthesized
  misc_feature (1)..(9): Cys and Cys form a ring
CPVSSMPRC

SEQ ID NO: 20 - protein, 9 aa, Artificial Sequence
  Chemically synthesized
  misc_feature (1)..(9): Cys and Cys form a ring
CQSPMPRLC

SEQ ID NO: 21 - protein, 9 aa, Artificial Sequence
  Chemically synthesized
  misc_feature (1)..(9): Cys and Cys form a ring
CLTPMPRLC

SEQ ID NO: 22 - protein, 15 aa, Artificial Sequence
  Chemically synthesized
  misc_feature (5)..(12): Cys and Cys form a ring
DSGLCMPRLRGCDPR

SEQ ID NO: 23 - protein, 15 aa, Artificial Sequence
  Chemically synthesized
TPSAHAMALQSLSVG

SEQ ID NO: 24 - protein, 15 aa, Artificial Sequence
  Chemically synthesized
  misc_feature (1)..(1): N-acetylated
  misc_feature (5)..(12): Cys and Cys form a ring
  misc_feature (15)..(15): terminus is C(=O)NH2
DSGLCMPRLRGCDPR

SEQ ID NO: 25 - protein, 8 aa, Artificial Sequence
  Chemically synthesized
  misc_feature (1)..(8): Cys and Cys form a ring
  misc_feature (1)..(1): N-propionylated
  misc_feature (8)..(8): terminus is C(=O)NH2
CMPRLRGC

SEQ ID NO: 26 - protein, 8 aa, Artificial Sequence
  Chemically synthesized
  misc_feature (1)..(8): Cys and Cys form a ring
  misc_feature (1)..(1): N-propionylated D-amino acid
  misc_feature (8)..(8): terminus is C(=O)NH2
CMPRLRGC

SEQ ID NO: 27 - protein, 8 aa, Artificial Sequence
  Chemically synthesized
  misc_feature (1)..(8): Cys and Pen form a ring
  misc_feature (1)..(1): N-propionylated D-amino acid
  misc_feature (3)..(3): Xaa = thiazolidine-4-carboxylic acid
  misc_feature (8)..(8): Xaa = Penicillamine
  misc_feature (8)..(8): terminus is C(=O)NH2
CMXRLRGX

SEQ ID NO: 28 - protein, 7 aa, Artificial Sequence
  Chemically synthesized
  misc_feature (1)..(7): Cys and Cys form a ring
  misc_feature (1)..(1): N-acetylated
  misc_feature (7)..(7): terminus is C(=O)NH2
CMPRLGC

SEQ ID NO: 29 - protein, 8 aa, Artificial Sequence
  Chemically synthesized
  misc_feature (1)..(8): Cys and Cys form a ring
  misc_feature (1)..(1): N-acetylated D-amino acid
  misc_feature (8)..(8): terminus is C(=O)NH2
CMPRLRGC

SEQ ID NO: 30 - protein, 9 aa, Artificial Sequence
  Chemically synthesized
  misc_feature (1)..(1): N-acetylated
  misc_feature (2)..(2): Xaa = Penicillamine
  misc_feature (2)..(9): Pen and Cys form a ring
  misc_feature (4)..(4): Xaa = thiazolidine-4-carboxylic acid
  misc_feature (9)..(9): terminus is C(=O)NH2
DXMXRLRGC

SEQ ID NO: 31 - protein, 8 aa, Artificial Sequence
  Chemically synthesized
  misc_feature (1)..(1): N-propionylated D-amino acid
  misc_feature (1)..(8): Cys and Pen form a ring
  misc_feature (3)..(3): Xaa = thiazolidine-4-carboxylic acid
  misc_feature (8)..(8): Xaa =Penicillamine, terminus is C(=O)NH2
CMXRLRGX

SEQ ID NO: 32 - protein, 8 aa, Artificial Sequence
  Chemically synthesized
  misc_feature (1)..(1): N-propionylated D-amino acid
  misc_feature (1)..(8): Cys and Pen form a ring
  misc_feature (3)..(3): Xaa = thiazolidine-4-carboxylic acid
  misc_feature (7)..(7): Xaa = Sarcosine
  misc_feature (8)..(8): Xaa = Penicillamine, terminus is C(=O)NH2
CMXRLRXX

SEQ ID NO: 33 - protein, 8 aa, Artificial Sequence
  Chemically synthesized
  misc_feature (1)..(1): N-propionylated D-amino acid
  misc_feature (1)..(8): Cys and Cys form a ring
  misc_feature (3)..(3): Xaa = Pipecolic group
  misc_feature (7)..(7): Xaa = Sarcosine
  misc_feature (8)..(8): terminus is C(=O)NH2
CMXRLRXC

SEQ ID NO: 34 - protein, 8 aa, Artificial Sequence
  Chemically synthesized
  misc_feature (1)..(8): Cys and Pen form a ring
  misc_feature (1)..(1): N-propionylated D-amino acid
  misc_feature (3)..(3): Xaa = Pipecolic group
  misc_feature (8)..(8): Xaa = penicillamine, terminus is C(=O)NH2
CMXRLRGX

SEQ ID NO: 35 - protein, 8 aa, Artificial Sequence
  Chemically synthesized
  misc_feature (1)..(1): N-propionylated D-amino acid
  misc_feature (1)..(8): Cys and Pen form a ring
  misc_feature (3)..(3): Xaa = Pipecolic group
  misc_feature (7)..(7): Xaa = sarcosine
  misc_feature (8)..(8): Xaa =  penicillamine, terminus is C(=O)NH2
CMXRLRXX

SEQ ID NO: 36 - protein, 16 aa, Artificial Sequence
  Chemically synthesized
  misc_feature (4)..(13): Cys and Cys form a ring
TDTCLMLPLLLGCDEE

SEQ ID NO: 37 - protein, 16 aa, Artificial Sequence
  Chemically synthesized
  misc_feature (4)..(13): Cys and Cys form a ring
DPICWYFPRLLGCTTL

SEQ ID NO: 38 - protein, 16 aa, Artificial Sequence
  Chemically synthesized
  misc_feature (4)..(13): Cys and Cys form a ring
WYPCYIYPRLLGCDGD

SEQ ID NO: 39 - protein, 16 aa, Artificial Sequence
  Chemically synthesized
  misc_feature (4)..(13): Cys and Cys form a ring
GNICMLIPGLLGCSYE

SEQ ID NO: 40 - protein, 16 aa, Artificial Sequence
  Chemically synthesized
  misc_feature (4)..(13): Cys and Cys form a ring
VNSCLLLPNLLGCGDD

SEQ ID NO: 41 - protein, 16 aa, Artificial Sequence
  Chemically synthesized
  misc_feature (4)..(13): Cys and Cys form a ring
TPVCILLPSLLGCDTQ

SEQ ID NO: 42 - protein, 16 aa, Artificial Sequence
  Chemically synthesized
  misc_feature (4)..(13): Cys and Cys form a ring
TVLCSLWPELLGCPPE

SEQ ID NO: 43 - protein, 16 aa, Artificial Sequence
  Chemically synthesized
  misc_feature (4)..(13): Cys and Cys form a ring
TFSCLMWPWLLGCESL

SEQ ID NO: 44 - protein, 16 aa, Artificial Sequence
  Chemically synthesized
  misc_feature (4)..(13): Cys and Cys form a ring
FGTCYTWPWLLGCEGF

SEQ ID NO: 45 - protein, 16 aa, Artificial Sequence
  Chemically synthesized
  misc_feature (4)..(13): Cys and Cys form a ring
SLFCRLLLTPVGCVSQ

SEQ ID NO: 46 - protein, 16 aa, Artificial Sequence
  Chemically synthesized
HLLVLPRGLLGCTTLA

SEQ ID NO: 47 - protein, 16 aa, Artificial Sequence
  Chemically synthesized
  misc_feature (4)..(13): Cys and Cys form a ring
TSLCSMFPDLLGCFNL

SEQ ID NO: 48 - protein, 16 aa, Artificial Sequence
  Chemically synthesized
  misc_feature (4)..(13): Cys and Cys form a ring
SHPCGRLPMLLGCAES

SEQ ID NO: 49 - protein, 17 aa, Artificial Sequence
  Chemically synthesized
TSTCSMVPGPLGAVSTW

SEQ ID NO: 50 - protein, 16 aa, Artificial Sequence
  Chemically synthesized
  misc_feature (4)..(13): Cys and Cys form a ring
KDPCTRWAMLLGCDGE

SEQ ID NO: 51 - protein, 16 aa, Artificial Sequence
  Chemically synthesized
  misc_feature (4)..(13): Cys and Cys form a ring
IMTCSVYPFLLGCVDK

SEQ ID NO: 52 - protein, 16 aa, Artificial Sequence
  Chemically synthesized
  misc_feature (4)..(13): Cys and Cys form a ring
IHSCAHVMRLLGCWSR

SEQ ID NO: 53 - protein, 10 aa, Artificial Sequence
  Chemically synthesized
CGGGPFWWWP

SEQ ID NO: 54 - protein, 10 aa, Artificial Sequence
  Chemically synthesized
CGGGHKYLRW

SEQ ID NO: 55 - protein, 10 aa, Artificial Sequence
  Chemically synthesized
CGGGKRIFMV

SEQ ID NO: 56 - protein, 10 aa, Artificial Sequence
  Chemically synthesized
CGGGKWHYLR

SEQ ID NO: 57 - protein, 12 aa, Artificial Sequence
  Chemically synthesized
THRPPMWSPVWP

SEQ ID NO: 58 - protein, 12 aa, Artificial Sequence
  Chemically synthesized
THRPPMWSPVWP

SEQ ID NO: 59 - protein, 12 aa, Artificial Sequence
  Chemically synthesized
THRPPMWSPVWP

SEQ ID NO: 60 - protein, 15 aa, Artificial Sequence
  Chemically synthesized
LSLERFLRCWSDAPA

SEQ ID NO: 61 - protein, 13 aa, Artificial Sequence
  Chemically synthesized
LERFLRCWSDAPA

SEQ ID NO: 62 - protein, 11 aa, Artificial Sequence
  Chemically synthesized
RFLRCWSDAPA

SEQ ID NO: 63 - protein, 9 aa, Artificial Sequence
  Chemically synthesized
LRCWSDAPA

SEQ ID NO: 64 - protein, 7 aa, Artificial Sequence
  Chemically synthesized
CWSDAPA

SEQ ID NO: 65 - protein, 18 aa, Artificial Sequence
  Chemically synthesized
DWFKAFYDKVAEKFKEAF

SEQ ID NO: 66 - protein, 13 aa, Artificial Sequence
  Chemically synthesized
  misc_feature (10)..(10): Phe phenyl is para substituted with benzoyl
DCAWHLGELFWCT

SEQ ID NO: 67 - protein, 7 aa, Artificial Sequence
  Chemically synthesized
HAIYPRH

(19) United States
(12) Patent Application Publication
(10) Pub. No.: US 2023/0090282 A1
(43) Pub. Date: Mar. 23, 2023

(54) ENGINEERED ANTIBODIES AS MOLECULAR DEGRADERS THROUGH CELLULAR RECEPTORS

(71) Applicant: YALE UNIVERSITY, New Haven, CT (US)

(72) Inventors: David SPIEGEL, Hamden, CT (US); David CAIANIELLO, Brooklyn, NY (US); Emily BRANHAM, Hamden, CT (US)

(21) Appl. No.: 17/654,984

(22) Filed: Mar. 15, 2022

Related U.S. Application Data
(63) Continuation-in-part of application No. PCT/US2020/055053, filed on Oct. 9, 2020.
(60) Provisional application No. 62/913,679, filed on Oct. 10, 2019.

(51) Int. Cl. A61K 47/68

(52) U.S. Cl. CPC A61K 47/6889; A61K 47/6807; A61K 47/6811

(57) ABSTRACT

The present disclosure provides, in one aspect, bifunctional compounds that can be used to promote or enhance degradation of certain circulating proteins. In certain embodiments, the circulating protein mediates a disease and/or disorder in a subject, and treatment or management of the disease and/or disorder requires degradation, removal, or reduction in concentration of the circulating protein in the subject. Thus, in certain embodiments, administration of a compound of the disclosure to the subject removes or reduces the circulation concentration of the circulating protein, thus treating, ameliorating, or preventing the disease and/or disorder.

### CROSS-REFERENCE TO RELATED APPLICATIONS

This application is a continuation-in-part of, and claims priority to, International Application No. PCT/US2020/055053, filed Oct. 9, 2020, which claims priority under 35 U.S.C. § 119(e) to U.S. Provisional Patent Application No. 62/913,679, filed Oct. 10, 2019, which is incorporated herein by reference in its entirety.

### STATEMENT REGARDING FEDERALLY SPONSORED RESEARCH OR DEVELOPMENT

This invention was made with government support under GM067543 awarded by National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM

This invention contains one or more sequences in a computer readable format in an accompanying text file titled “047162-7249US1_sequence_listing,” which is 21.8 KB in size and was created on Nov. 23, 2021, the contents of which are incorporated herein by reference in their entirety.

### BACKGROUND OF THE DISCLOSURE

Among the mechanisms that regulate transport of molecules into a cell is receptor-mediated endocytosis. In this process, a receptor on the cell surface binds to a specific ligand (or a molecule comprising such specific ligand) that is present outside the cell—this ligand may be a small molecule, metabolite, hormone, protein, or even a virus. The binding process triggers the inward budding of the plasma membrane (invagination), forming a vesicle containing the receptor-ligand complex. The vesicle becomes an endosome and subsequently fuses with lysosomes, and the receptor is degraded along with ligand cargo bound thereto or the receptor is recycled to the cell surface for further harvesting of the circulating ligand.

One such receptor is the asialoglycoprotein receptor (ASGPR). This receptor is a C-type lectin, and its major biological role is to bind, internalize, and subsequently clear from circulation glycoproteins that contain terminal galactose or N-acetylgalactosamine residues (asialoglycoproteins). ASGPRs remove the target glycoproteins from circulation through endocytosis and subsequent lysosomal degradation. ASGPRs are highly expressed on the surface of hepatocytes, several human carcinoma cell lines, and liver cancers, and also weakly expressed by glandular cells of the gallbladder and the stomach. These receptors are known to be involved in the clearance of IgG subtypes and other antibody isotypes from circulation, removal of apoptotic cells, clearance of low density lipoprotein (LDL) and chylomicron remnants, and disposal of cellular fibronectin.

There is a need in the art for novel compounds and methods that allow for inhibition, removal, and/or degradation of certain extracellular proteins that mediate a disease and/or disorder in a subject. The present disclosure addresses this need.

### BRIEF SUMMARY OF THE DISCLOSURE

The present disclosure provides a compound comprising formula (I), or a salt, geometric isomer, stereoisomer, or solvate thereof:

[Ab]_{k′}—[CON]ₕ-[Linker]ᵢ-[CON]_{h′}—[CRBM]_{j′} (I),

wherein Ab, CON, Linker, CRBM, k′, h, i, h′, and j′ are defined elsewhere herein.

The present disclosure further provides a pharmaceutical composition comprising at least one compound contemplated herein and at least one pharmaceutically acceptable excipient.

The present disclosure further provides a method of treating a disease or disorder in a subject, the method comprising administering a therapeutically effective amount of at least one compound contemplated herein.

### BRIEF DESCRIPTION OF THE FIGURES

The drawings illustrate generally, by way of example, but not by way of limitation, various embodiments of the present application.

As used herein, the term “REAG” refers to any reagent comprising —CON, -Linker, —CON-Linker, -Linker-CON, —CON-Linker-CON, -CRBM, —CON-CRBM, -Linker-CRBM, —CON-Linker-CRBM, -Linker-CON-CRBM, and/or —CON-Linker-CON-CRBM.

FIG. 1 illustrates a non-limiting preparation of a compound of the disclosure comprising a mannose receptor binder.

FIG. 2 illustrates a non-limiting preparation of a compound of the disclosure comprising a mannose receptor binder.

FIG. 3 illustrates a non-limiting preparation of a compound of the disclosure comprising a mannose receptor binder.

FIG. 4 illustrates a non-limiting preparation of a compound of the disclosure comprising a mannose receptor binder.

FIG. 5 illustrates a non-limiting preparation of a compound of the disclosure comprising a mannose receptor binder.

FIG. 6 illustrates a non-limiting preparation of a compound of the disclosure comprising a mannose receptor binder.

FIG. 7 illustrates a non-limiting preparation of a compound of the disclosure comprising a mannose receptor binder.

FIG. 8 illustrates a non-limiting preparation of a polymeric compound comprising mannose-6-phosphate receptor binders.

FIG. 9 illustrates non-limiting examples of R¹ and/or R³ groups in ASGPRBM.

FIG. 10 illustrates non-limiting examples of R² groups in ASGPRBM.

FIGS. 11A-11B illustrate the non-limiting synthesis of an ASGPRBM group.

FIGS. 12A-12C illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

FIGS. 13A-13L illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

FIGS. 14A-14O illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

FIG. 15 illustrates non-limiting synthetic schemes that allow for labeling (derivatization) of an antibody (labeled as Ab) with an azido group.

FIG. 16 illustrates a non-limiting synthetic scheme that allow for labeling (derivatization) of a CRBM group with a strained alkyne containing group. In certain non-limiting embodiments, any azido-containing compound (such as, but not limited to, those shown in FIG. 15) can react with a strained alkyne-containing compound (such as, but not limited to, that shown in FIG. 16), so as to generate a compound of the disclosure.

### DETAILED DESCRIPTION OF THE DISCLOSURE

The present disclosure provides, in one aspect, bifunctional compounds that can be used to promote or enhance degradation of an extracellular protein (which may be, for example, a circulating protein and/or a cell surface protein, which can be attached or embedded in the cell membrane). In certain embodiments, the extracellular protein mediates a disease and/or disorder in a subject, and treatment or management of the disease and/or disorder requires degradation, removal, or reduction in concentration of the extracellular protein in the subject. Thus, in certain embodiments, administration of a compound of the disclosure to the subject removes the extracellular protein and/or reduces the circulation concentration of the extracellular protein, thus treating, ameliorating, or preventing the disease and/or disorder in the subject.

In certain embodiments, the compound of the disclosure comprises an antibody, such as but not limited to a monoclonal antibody, which can bind to an extracellular protein of interest. In other embodiments, the compound of the disclosure further comprises another group (such as but not limited to a small molecule) that binds to a cellular receptor, whereby the binding leads to endocytosis of the compound (and/or the extracellular protein-compound complex). The receptor binder and the antibody can be linked via a linker such as a polyethylene glycol (PEG), any other linker as described herein with adjustable length, or other linker as described herein and containing contains one or more connector molecule(s), which are referred to herein as CON. The conjugation of the antibody to the connector molecule can be accomplished using any chemistry known to one skilled in the art, such as but not limited to lysine bioconjugation using activated esters, such as but not limited to NHS esters. Once the extracellular protein-compound complex undergoes endocytosis, the extracellular protein is eventually degraded, and the compound may be degraded or recycled to the outside of the cell.

In certain embodiments, the receptor is a hepatocyte asialoglycoprotein receptor (ASGPR). In that case, the binding moiety is referred to herein as ASGPR binding moiety, or ASGPRBM. The disclosure is not limited to the receptor, but rather contemplates the use of other receptor described herein or any other endocytic receptor known in the art.

Further, the disclosure is not limited to degradation performed in hepatocytes. Rather, the disclosure contemplates that non-hepatic cells in the body display certain degradation receptors, and those receptors are contemplated within the present disclosure.

In one aspect, the compounds of the disclosure bind to extracellular target proteins and cause them to be removed from circulation in the body (and from the body) through the liver. Thus, the compounds of the disclosure harness the body's own machinery for degrading proteins. Without wishing to be limited by any theory, the compounds of the disclosure bind to certain receptors located in certain cells, such as but not limited to hepatocytes, such as but not limited to ASGPR. Such binding triggers degradation of protein targets via endolysosomal proteolysis. As a consequence of this mechanism, there is a lowering in the circulating levels of the extracellular protein target. As a result, the corresponding disease symptoms are attenuated and/or eliminated from the subject administered the present compounds.

The ASPGR has the function of clearing desialylated glycoproteins with exposed non-reducing D-galactose (Gal) or N-acetylgalactosamine (GalNac) as end groups. ASGPR is expressed at a level of about 500,000 per hepatocyte, and has minimal existence elsewhere in the body. Internalization of the target glycoproteins by the ASGPR has a half-life of about 3 min. The presently claimed bifunctional compounds selectively bind to the extracellular protein through the compound's antibody moiety, thus forming a protein complex. When this protein complex reaches the liver, the asialoglycoprotein receptor binding moiety (ASGPRBM) of the molecule engages the end-lysosomal pathway of hepatocytes through the ASGPR. Endosomal bound ASPGR releases the extracellular protein ligand at pH 5.4, and the ligand is eliminated from circulation by the hepatocytes. However, the ASPGR remains available for recycling; it is spared from lysosomal degradation and buds into recycling endosomes. Indeed, it can be recycled up to about 200 times with a recycling rate of about 15-20 minutes, depending on the cell line. ASPGR has a very promiscuous ligand size requirement, most likely reaching diameters of about 70 nm. For comparison, the IgM pentamer is approximately 20 nm in diameter, and thus meets the ASPGR's ligand size requirement.

The disclosures of the International Patent Applications No. PCT/US2019/026260, filed Apr. 8, 2019 (and published as WO 2019/199634 on Oct. 17, 2019), and No. PCT/US2019/026239, filed Apr. 8, 2019 (and published as WO 2019/199621 on Oct. 17, 2019), are incorporated herein in their entireties by reference.

Without wishing to be limited by any theory, the use of antibodies, such as but not limited to monoclonal antibodies, within the compounds of the disclosure has distinctive advantages over similar bifunctional compounds in the prior art.

In one aspect, the affinity of an antibody for its target is often in the pM to low nM range, which corresponds to particularly strong binding. In contrast, small molecule protein binders often have nM to μM range affinities for their targets. Thus, antibodies are expected to have much tighter binding to the molecular target of interest.

In another aspect, antibodies, such as but not limited to monoclonal antibodies, are very specific to their intended target, which decreases their toxicity due to off-target interactions.

In yet another aspect, antibodies interact with the neonatal Fc receptor (FcRn), also known as the Brambell receptor. In humans, the FcRn regulates IgG and serum albumin turnover. FcRn extends the half-life of IgG and serum albumin by reducing lysosomal degradation in endothelial cells and bone-marrow derived cells. IgG, serum albumin, and other serum proteins are continuously internalized through pinocytosis, and would presumably be degraded in the lysosome. However, IgG and serum albumin are bound by FcRn at slightly acidic pH (<6.5), and recycled to the cell surface where they are released at the neutral pH (>7.0) of blood, thus avoiding lysosomal degradation. Interestingly, both FcRn and ASGPR are expressed on hepatocytes, the primary cell type in which ASGPR-mediated degradation occurs. Thus, the compounds of the disclosure can bind to ASGPRBM and yet escape ASGPR-mediated degradation through binding to FcRn.

The rescue of compounds of the disclosure by FcRn provides several therapeutic advantages. Generally, compounds comprising a circulating protein small-molecule binding partner are degraded along with the circulating protein of interest. In contrast, the modified antibodies of the disclosure can participate in multiple rounds of protein binding and targeting to degradation due to their recycling via FcRn. Further, as the compounds of the disclosure can participate in multiple rounds of degradation, they can be administered at lower doses, which is therapeutically advantageous. Further, as the half-life of the compounds of the disclosure is extended via FcRn rescue, their dosing frequency is decreased relative to the dosing frequency necessary when using compounds comprising a circulating protein small-molecule binding partner.

In fact, the antibody-containing compounds of the disclosure have advantageous properties not only over the previously described compounds comprising a circulating protein small-molecule binding partner, but also over the non-derivatized antibodies themselves. The main function of unmodified antibodies is to bind to and neutralize their protein targets—in this situation, unmodified antibodies can participate in one binding event each. In contrast, the antibody-containing compounds of the disclosure work by degrading protein targets, and some percent of these compounds are recycled by FcRn and are available to participate in another round of binding after a degradation event occurs. These available antibody-containing compounds of the disclosure can thus participate in semi-catalytic protein degradation, a property which is facilitated by FcRn mediated recycling.

As an additional advantage, the present disclosure allows for efficient generation of novel antibody-containing compounds, wherein an ASGPRBM is attached to any antibody of choice (without the need to alter the amino acid sequence of those antibodies and/or extensively test the impact of those amino acid changes on FcRn binding, protein binding, and protein release). This ease of synthesis lends itself to the examination of the degradation of various protein targets. In certain embodiments, using this method is less expensive than traditional antibody modification methods. In certain embodiments, a less frequent dosing regimen for the antibody-containing compounds of the disclosure (as compared to other antibody therapies) decreases the overall therapy cost, lowering financial burdens on insurance companies and patients.

In accordance with the present disclosure, conventional chemical synthetic and pharmaceutical formulation methods, as well as pharmacology, molecular biology, microbiology, and recombinant DNA techniques within the skill of the art may be employed. Such techniques are well-known and are otherwise explained fully in the literature.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics that are normal or expected for one cell or tissue type, might be abnormal for a different cell or tissue type.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂CC(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino acid sequence variant” refers to polypeptides having amino acid sequences that differ to some extent from a native sequence polypeptide. Ordinarily, amino acid sequence variants possess at least about 70% homology, at least about 80% homology, at least about 90% homology, or at least about 95% homology to the native polypeptide. The amino acid sequence variants possess substitutions, deletions, and/or insertions at certain positions within the amino acid sequence of the native amino acid sequence.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aminoalkyl” as used herein refers to amine connected to an alkyl group, as defined herein. The amine group can appear at any suitable position in the alkyl chain, such as at the terminus of the alkyl chain or anywhere within the alkyl chain.

The term “antibody,” as used herein, refers to an immunoglobulin molecule that specifically binds with an antigen. Antibodies can be intact immunoglobulins derived from natural sources or from recombinant sources, and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, single-domain antibodies such as sdAb (either VL or VH), such as camelid antibodies (Riechmann, 1999, J. Immunol. Meth. 231:25-38), camelid VHH domains, composed of either a VL or a VH domain that exhibit sufficient affinity for the target, and multispecific antibodies formed from antibody fragments such as a bivalent fragment comprising two Fab fragments linked by a disulfide bridge at the hinge region, and an isolated complementarity-determining region (CDR) or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger & Hudson, 2005, Nature Biotech. 23:1126-1136). Antigen binding fragments can also be grafted into scaffolds based on polypeptides such as a fibronectin type III (Fn3) (U.S. Pat. No. 6,703,199, which describes fibronectin polypeptide minibodies). The antibody fragment also includes a human antibody or a humanized antibody or a portion of a human antibody or a humanized antibody.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, the term “asialoglycoprotein receptor binding moiety” or “ASGPRBM” refers to a group that is capable of binding to at least one hepatocyte asialoglycoprotein receptor on the surface of a cell, such as but not limited to hepatocytes. Once the ASGPRBM, and any additional moiety to which it is attached, binds to the receptor on the surface of hepatocyte, the molecule comprising the ASGPRBM is taken into the hepatocyte via a phagocytosis mechanism wherein the molecule is at least partially degraded through lysosomal degradation.

As used herein, the term “C₆₋₁₀-C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀-C₆₋₁₀ biaryl include biphenyl and binaphthyl.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Eₘₐₓ) achieved within an assay.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

As used herein, the term “GN3” refers to the group

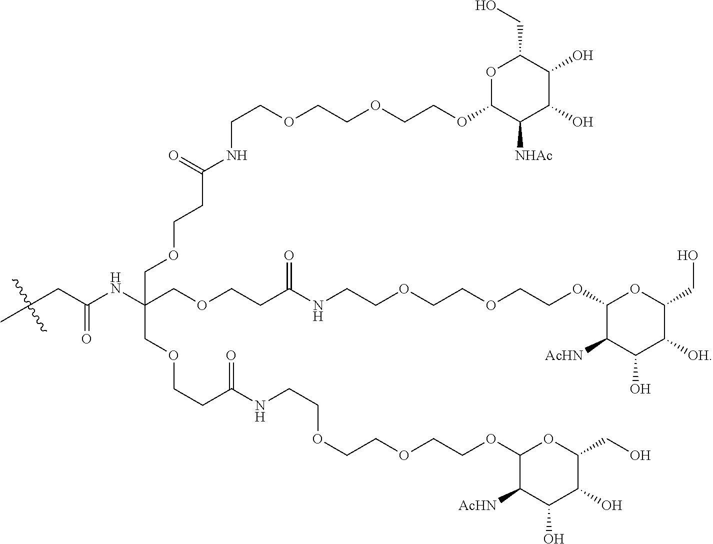 [SMILES: *CC(=O)NC(COCCC(=O)NCCOCCOCCOC1O[C@H](CO)[C@H](O)[C@H](O)[C@H]1NC(C)=O)(COCCC(=O)NCCOCCOCCO[C@@H]1O[C@H](CO)[C@H](O)[C@H](O)[C@H]1NC(C)=O)COCCC(=O)NCCOCCOCCO[C@@H]1O[C@H](CO)[C@H](O)[C@H](O)[C@H]1NC(C)=O]

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include:

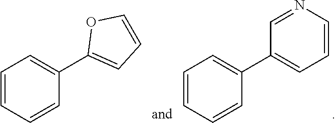 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, wherein X¹, X², and X³ are all different, wherein X¹ and X² are the same but X³ is different, and other analogous permutations.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

By the term “modulating,” as used herein, is meant mediating a detectable increase or decrease in the activity and/or level of a mRNA, polypeptide, or a response in a subject compared with the activity and/or level of a mRNA, polypeptide or a response in the subject in the absence of a treatment or compound, and/or compared with the activity and/or level of a mRNA, polypeptide, or a response in an otherwise identical but untreated subject. The term encompasses activating, inhibiting and/or otherwise affecting a native signal or response thereby mediating a beneficial therapeutic response in a subject, preferably, a human.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, the term “potency” refers to the dose needed to produce half the maximal response (ED₅₀).

As used herein, the term “REAG” refers to any reagent comprising —CON, -Linker, —CON-Linker, -Linker-CON, —CON-Linker-CON, -CRBM, —CON-CRBM, -Linker-CRBM, —CON-Linker-CRBM, -Linker-CON-CRBM, and/or —CON-Linker-CON-CRBM. As described herein, the REAG reacts with a Protein binder so as to incorporate the Protein binder in the compound of the disclosure, or a fragment thereof, derivative thereof, or intermediate thereto.

The term “room temperature” as used herein refers to a temperature of about 15° C. to 28° C.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “solvent” as used herein refers to a liquid that can dissolve a solid, liquid, or gas. Non-limiting examples of solvents are silicones, organic compounds, water, alcohols, ionic liquids, and supercritical fluids.

The term “standard temperature and pressure” as used herein refers to 20° C. and 101 kPa.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “autoimmune disease” refers to a disease or illness that occurs when the body tissues are attacked by its own immune system. Examples of autoimmune diseases include, for example, systemic lupus erythematosus, Sjogren syndrome, Hashimoto thyroiditis, rheumatoid arthritis, juvenile (type 1) diabetes, polymyositis, scleroderma, Addison's disease, vitiligo, pernicious anemia, glomerulonephritis, and pulmonary fibrosis, among numerous others.

A more complete list of autoimmune diseases which may be treated by compounds and pharmaceutical compositions according to the present disclosure includes Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn' disease, microscopic colitis, ulcerative colitis, autophospholipid syndrome (AP1S), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankolosing spondylitis, CREST syndrome, drug-induced lupus, enthesitis-related arthritis, esosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymyssitis, acute disseminated encephalomyelitis (ADEM), acute motor axonic neuropathy, anti-NMDA receptor encephalitis, Balo concentric sclerosis, Bickerstaff's encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosinophilic granulomatosis with polyangiitis (EGPA), giant cell arteritis, granulomatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency and pyoderma gangrenosum, among others.

The term “cancer” or “neoplasia” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present disclosure may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present disclosure.

Representative common cancers to be treated with compounds according to the present disclosure include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present disclosure. Because of the activity of the present compounds, the present disclosure has general applicability treating virtually any cancer in any tissue, thus the compounds, compositions and methods of the present disclosure are generally applicable to the treatment of cancer and in reducing the likelihood of development of cancer and/or the metastasis of an existing cancer.

In certain particular aspects of the present disclosure, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present disclosure is generally applicable and may be used to treat any cancer in any tissue, regardless of etiology.

The term “anticancer agent” or “additional anticancer agent” refers to a compound other than the chimeric compounds according to the present disclosure which may be used in combination with a compound according to the present disclosure for the treatment of cancer. Exemplary anticancer agents which may be co-administered in combination with one or more chimeric compounds according to the present disclosure include, for example, antimetabolites, inhibitors of topoisomerase I and II, alkylating agents and microtubule inhibitors (e.g., taxol), among others. Exemplary anticancer compounds for use in the present disclosure may include everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (MEK) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab (Arzerra), zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1 H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258, 345-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄—(C₂H₄O₂)_{X} wherein x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, vemurafenib among others, including immunotherapy agents such as IDO inhibitors (an inhibitor of indoleamine 2,3-dioxygenase (IDO) pathway) such as Indoximod (NLG-8187), Navoximod (GDC-0919) and NLG802, PDL1 inhibitors (an inhibitor of programmed death-ligand 1) including, for example, nivolumab, durvalumab and atezolizumab, PD1 inhibitors such as pembrolizumab (Merck) and CTLA-4 inhibitors (an inhibitor of cytotoxic T-lymphocyte associated protein 4/cluster of differentiation 152), including ipilimumab and tremelimumab, among others.

In addition to anticancer agents, a number of other agents may be co-administered with chimeric compounds according to the present disclosure in the treatment of cancer. These include active agents, minerals, vitamins and nutritional supplements which have shown some efficacy in inhibiting cancer tissue or its growth or are otherwise useful in the treatment of cancer. For example, one or more of dietary selenium, vitamin E, lycopene, soy foods, curcumin (turmeric), vitamin D, green tea, omega-3 fatty acids and phytoestrogens, including beta-sitosterol, may be utilized in combination with the present compounds to treat cancer.

The term “inflammatory disease” is used to describe a disease or illness with acute, but more often chronic inflammation as a principal manifestation of the disease or illness. Inflammatory diseases include diseases of neurodegeneration (including, for example, Alzheimer's disease, Parkinson's disease, Huntington's disease; other ataxias), diseases of compromised immune response causing inflammation (e.g., dysregulation of T cell maturation, B cell and T cell homeostasis, counters damaging inflammation), chronic inflammatory diseases including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, pancreatic β-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), elevated triglycerides and metabolic syndrome, liver disease, renal disease (apoptosis in plaques, glomerular disease), cardiovascular disease (especially including infarction, ischemia, stroke, pressure overload and complications during reperfusion), muscle degeneration and atrophy, low grade inflammation, gout, silicosis, atherosclerosis and associated conditions such as cardiac and neurological (both central and peripheral) manifestations including stroke, age-associated dementia and sporadic form of Alzheimer's disease, and psychiatric conditions including depression), stroke and spinal cord injury, arteriosclerosis, among others. In these diseases, elevated MIF is very often observed, making these disease states and/or conditions response to therapy using compounds and/or pharmaceutical compositions according to the present disclosure. It is noted that there is some overlap between certain autoimmune diseases and inflammatory diseases as described herein.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

The disclosure provides a compound comprising formula (I), or a salt, geometric isomer, stereoisomer, or solvate thereof:

[Ab]_{k′}—[CON]ₕ-[Linker]ᵢ-[CON]_{h′}—[CRBM]_{j′} (I).

In certain embodiments, the compound comprises formula (Ia), or a salt, geometric isomer, stereoisomer, or solvate thereof:

[Ab]-[CON]₀₋₁-[Linker]-[CON]₀₋₁-[CRBM] (Ia).

In (I) or (Ia), the Ab is an antibody, such as but not limited to a monoclonal antibody, which binds to a biological target, such as but not limited to an extracellular protein, such as but not limited to a circulating protein and/or a cell surface protein. In certain embodiments, the circulating protein mediates a disease and/or disorder in a subject, and treatment or management of the disease and/or disorder requires degradation, removal, or reduction in concentration of the circulating protein in the subject. In certain embodiments, the Ab within (I) or (Ia) is capable of binding to the circulating protein in the plasma of the subject with identical affinity or substantially similar affinity as compared to the Ab itself.

In (I) or (Ia), the CRBM is a cellular receptor binding moiety that binds to at least one receptor on the surface of hepatocytes or other degrading cells in the subject, whereby binding leads to endocytosis and degradation of (I) and/or (Ia) and/or the biological target. In certain embodiments, the CRBM is ASGPRBM, which is a cellular receptor binding moiety that binds to at least one asialoglycoprotein receptor on the surface of hepatocytes or other degrading cells in the subject.

In (I) or (Ia), each CON is independently a bond or a group that covalently links an Ab to a CRBM, an Ab to a Linker, and/or a Linker to a CRBM.

In (I) or (Ia), the Linker is a group having a valence ranging from 1 to 15. In certain embodiments, the valence of the Linker is 1 to 10. In certain embodiments, the valence of the Linker is 1 to 5. In certain embodiments, the valence of the Linker is 1, 2, or 3. In certain embodiments, the Linker covalently links one or more CRBM and/or Ab groups, optionally through a CON, wherein the Linker optionally itself contains one or more CON groups.

In certain embodiments, k′ is an integer ranging from 1 to 15. In certain embodiments, k′ is an integer ranging from 1 to 10. In certain embodiments, k′ is an integer ranging from 1 to 5. In certain embodiments, k′ is an integer ranging from 1 to 3. In certain embodiments, k′ is 1, 2 or 3.

In certain embodiments, j is an integer ranging from 1 to 15. In certain embodiments, j is an integer ranging from 1 to 10. In certain embodiments, j is an integer ranging from 1 to 5. In certain embodiments, j is an integer ranging from 1 to 3. In certain embodiments, j is 1, 2 or 3.

In certain embodiments, h is an integer ranging from 0 to 15. In certain embodiments, h is an integer ranging from 1 to 15. In certain embodiments, h is an integer ranging from 1 to 10. In certain embodiments, h is an integer ranging from 1 to 5. In certain embodiments, h is an integer ranging from 1 to 3. In certain embodiments, h is 1, 2, or 3.

In certain embodiments, h′ is an integer ranging from 0 to 15. In certain embodiments, h′ is an integer ranging from 1 to 15. In certain embodiments, h′ is an integer ranging from 1 to 10. In certain embodiments, h′ is an integer ranging from 1 to 5. In certain embodiments, h′ is an integer ranging from 1 to 3. In certain embodiments, h′ is 1, 2, or 3.

In certain embodiments, i is an integer ranging from 0 to 15. In certain embodiments, i is an integer ranging from 1 to 15. In certain embodiments, i is an integer ranging from 1 to 10. In certain embodiments, i is an integer ranging from 1 to 5. In certain embodiments, i is an integer ranging from 1 to 3. In certain embodiments, i is 1, 2, or 3.

In certain embodiments, at least one of h, h′, and i is at least 1.

In certain embodiments, k′, j′, h, h′, and i are each independently 1, 2, or 3.

In certain embodiments, k′ is 1, and j′ is 1, 2, or 3.

### CRBM

Folic Acid (Folate) Receptor:

In certain embodiments, the CRBM is folic acid, or any fragment or derivative thereof that is capable of binding to the folic acid (folate) receptor. Folate receptors bind folate and reduced folic acid derivatives and mediates delivery to the interior of cells of tetrahydrofolate, which is then converted from monoglutamate to polyglutamate forms (such as 5-methyltetrahydrofolate) as only monoglutamate forms can be transported across cell membranes. Human proteins from this family include folate receptor 1 (adult), folate receptor 2 (fetal), and folate receptor gamma.

In certain embodiments, the folic acid CRBM comprises methotrexate or a biologically active fragment thereof:

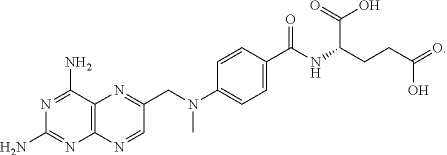 [SMILES: CN(Cc1cnc2nc(N)nc(N)c2n1)c1ccc(C(=O)N[C@@H](CCC(=O)O)C(=O)O)cc1]

In certain embodiments, the folic acid CRBM comprises premetrexed or a biologically active fragment thereof:

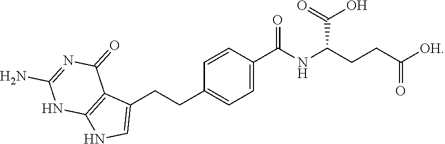 [SMILES: Nc1nc(=O)c2c(CCc3ccc(C(=O)N[C@@H](CCC(=O)O)C(=O)O)cc3)c[nH]c2[nH]1]

In certain embodiments, the folic acid CRBM can be incorporated into the compound of the disclosure through one of its carboxylic acid. In other embodiments, the folic acid CRBM can be incorporated into the compound of the disclosure using N-hydroxysuccinamidyl (NHS)-activated folate (similar chemistry is applicable to methotrexate and premetrexed).

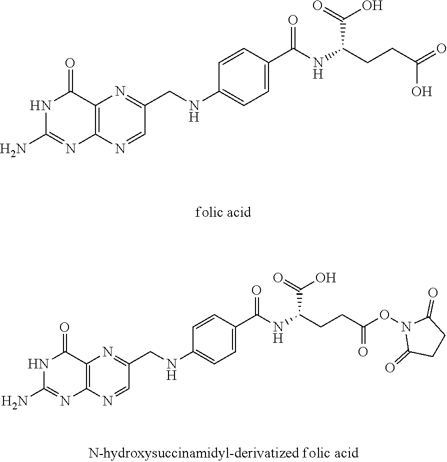 [SMILES: Nc1nc2ncc(CNc3ccc(C(=O)N[C@@H](CCC(=O)O)C(=O)O)cc3)nc2c(=O)[nH]1 ; Nc1nc2ncc(CNc3ccc(C(=O)N[C@@H](CCC(=O)ON4C(=O)CCC4=O)C(=O)O)cc3)nc2c(=O)[nH]1]

Mannose Receptor:

In certain embodiments, the CRBM is a group that binds to a mannose receptor. In certain embodiments, the CRBM comprises the group:

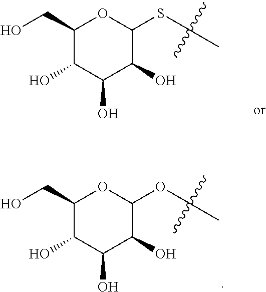 [SMILES: *SC1O[C@H](CO)[C@@H](O)[C@H](O)[C@@H]1O ; *OC1O[C@H](CO)[C@@H](O)[C@H](O)[C@@H]1O]

In certain embodiments, the mannose receptor CRBM can be attached to the compound of the disclosure (such as but not limited to the REAG) using one of the following reagents (which may be optionally protected with appropriately protecting groups):

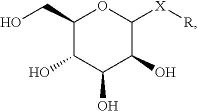

wherein X is S or O, wherein R is selected from the group consisting of:

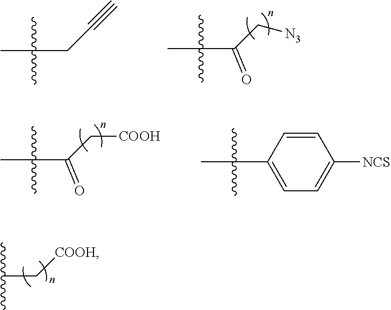

and wherein each occurrence of ‘n’ is independently 1, 2, 3, 4, 5, 6, 7, 8, 9, 10, 11, 12, 13, 14, 15, 16, 17, 18, 19, or 20.

In certain embodiments, the mannose receptor CRBM is part of a polymeric molecule. Such molecule can comprise one or more independently selected mannose receptor CRBMs as part of a polymeric chain. In certain embodiments, the CRBMs are incorporated into the polymeric molecule using CRBM reagents recited elsewhere herein.

Mannose-6-Phosphate (M6P) Receptor:

In certain embodiments, the CRBM is a group that binds to a mannose-6-phosphate (M6P) receptor. In certain embodiments, the CRBM comprises the group:

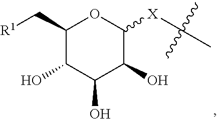

wherein X is O or S, and wherein R¹ is selected from the group consisting of:

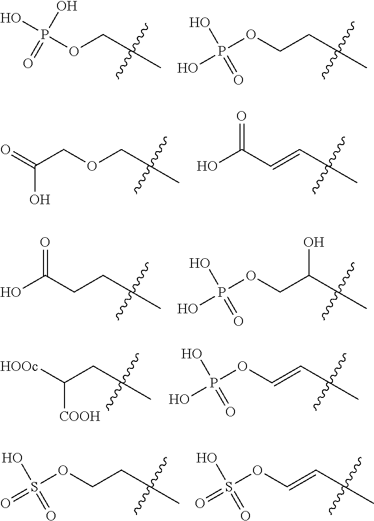

In certain embodiments, the CRBM can be attached to the compound of the disclosure (such as but not limited to the REAG) using one of the following reagents (which may be optionally protected with appropriately protecting groups):

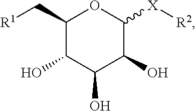

wherein X and R¹ are as defined elsewhere herein, wherein R² is selected from the group consisting of:

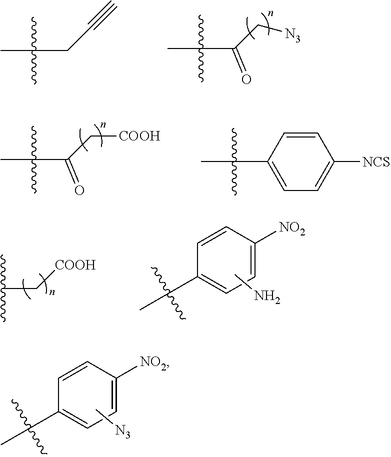

and wherein each occurrence of ‘n’ is independently 1, 2, 3, 4, 5, 6, 7, 8, 9, 10, 11, 12, 13, 14, 15, 16, 17, 18, 19, or 20.

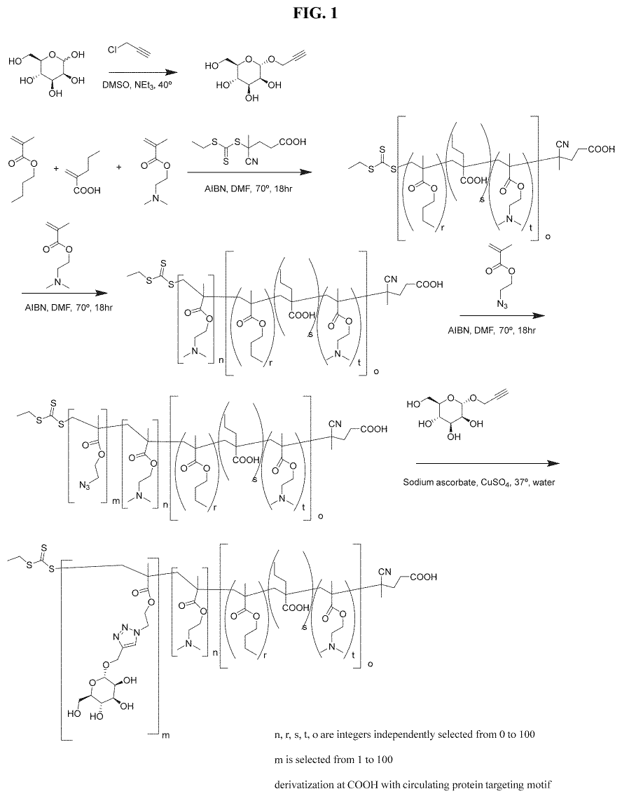

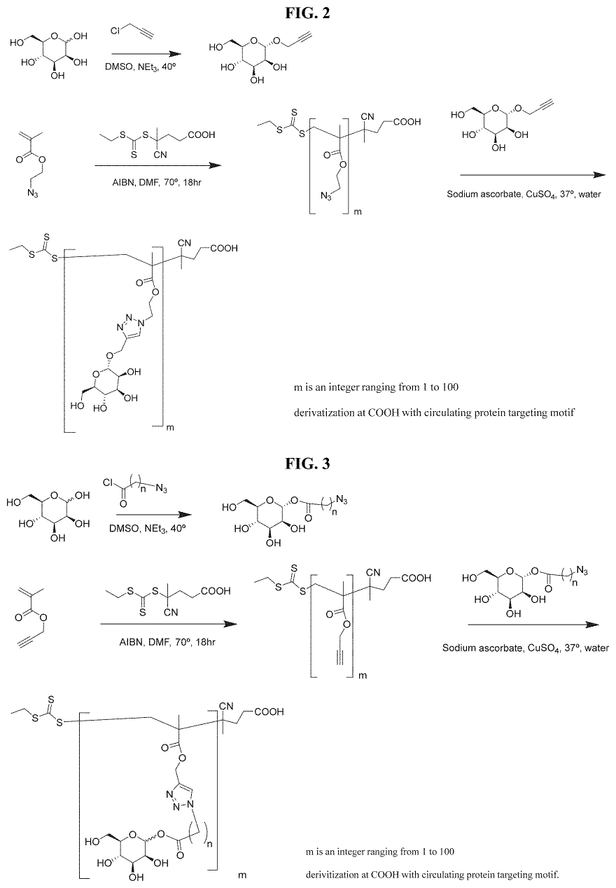

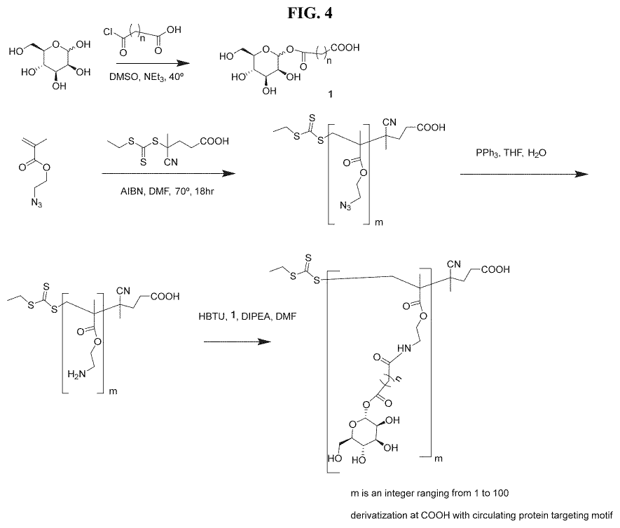

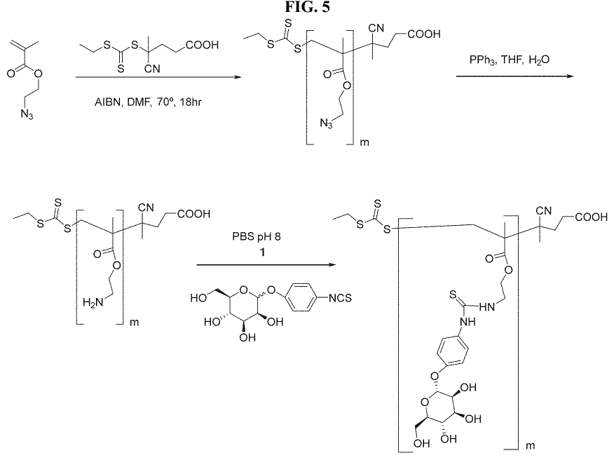

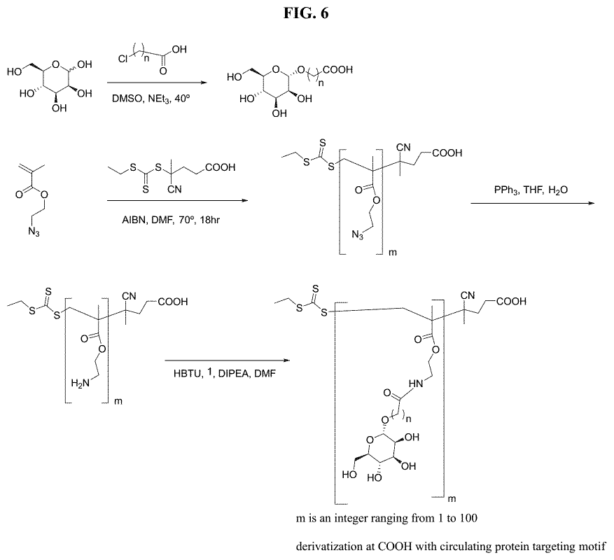

In certain embodiments, the M6P receptor CRBM is part of a polymeric molecule. Such molecule can comprise one or more independently selected M6P receptor CRBMs as part of a polymeric chain. In certain embodiments, the CRBMs are incorporated into the polymeric molecule using CRBM reagents recited elsewhere herein. FIGS. 1-8 illustrate exemplary mannose receptor binders and their preparation.

In certain embodiments, the M6P receptor CRBM is one of the following (Yamaguchi, et al., 2016, J. Am. Chem. Soc. 138(38):12472-12485):

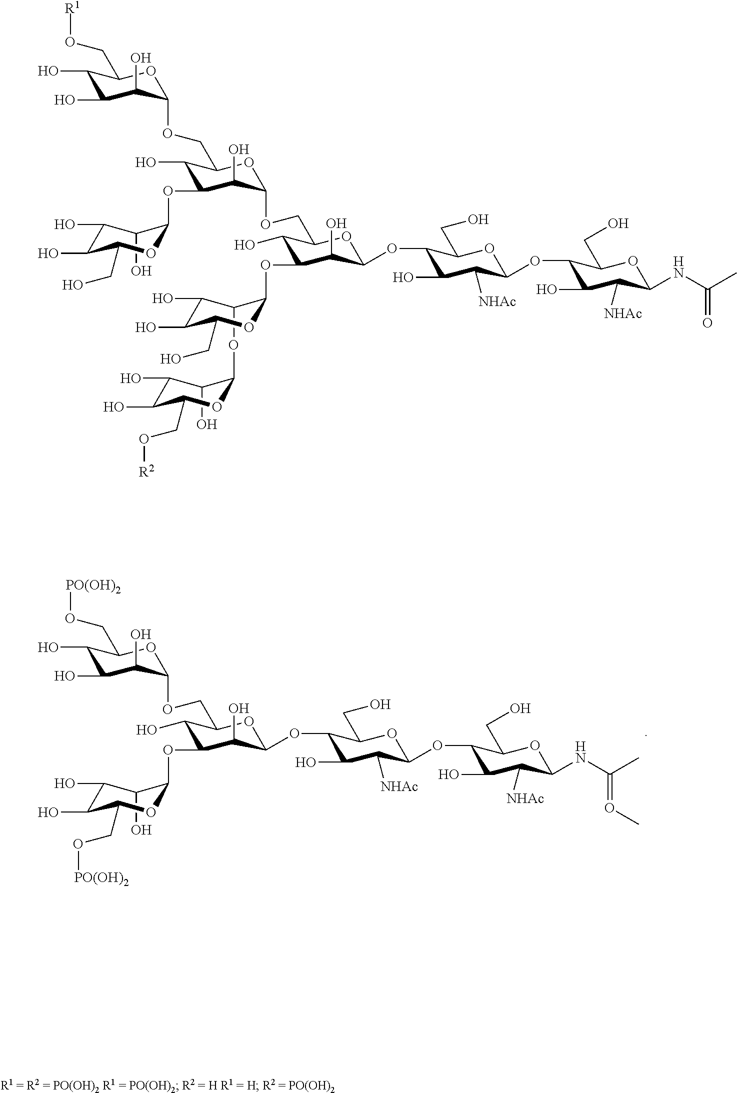

In certain embodiments, the M6P receptor CRBM is one of the following (US2011/0110960 to Platenburg):

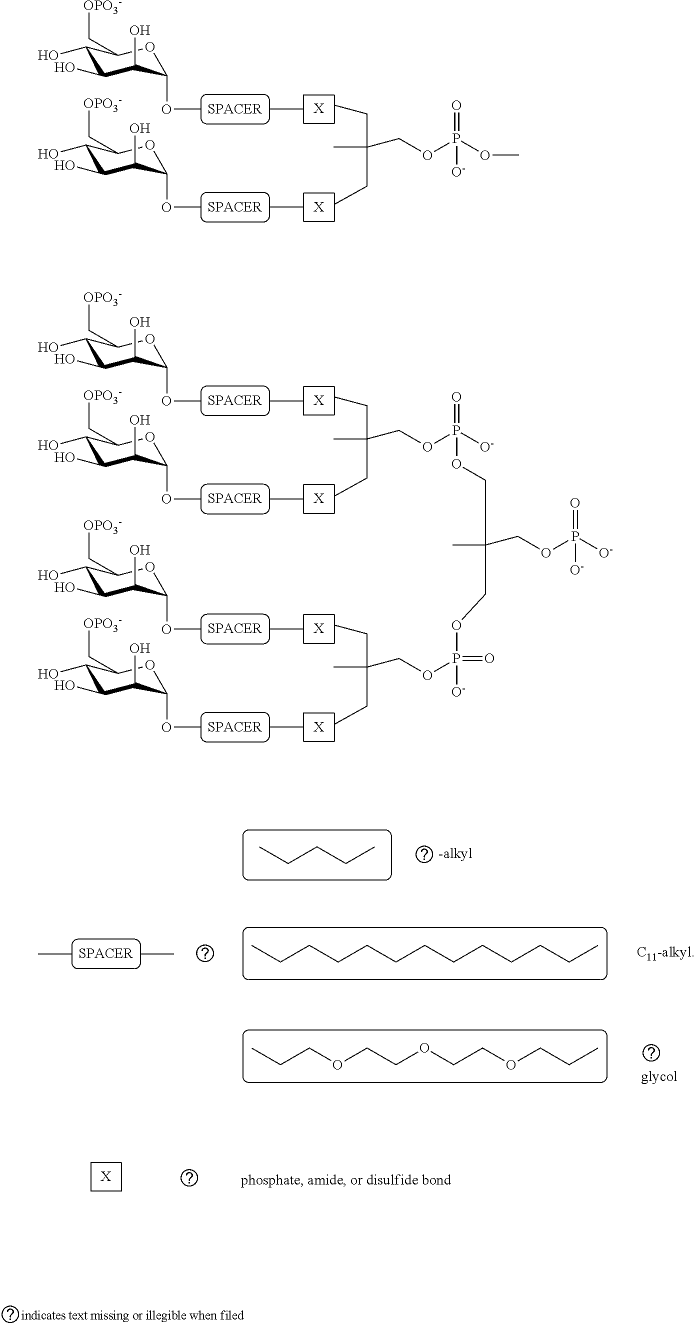

Low Density Lipoprotein Receptor-Related Protein 1 (LRP1) Receptor: In certain embodiments, the CRBM is a LRP1 [Low density lipoprotein receptor-related protein 1; also known as alpha-2-macroglobulin receptor (A2MR), apolipoprotein E receptor (APOER) or cluster of differentiation 91 (CD91)] binding group comprising one of the following amino acid sequences:
Ac-VKFNKPFVFLN1eIEQNTK-NH₂ (SEQ ID NO:1), Toldo et al., 2017, JACC: Basic to Translational Science 2.5:561-574;
VKFNKPFVFLMIEQNTK (SEQ ID NO:2), Toldo et al., 2017, JACC: Basic to Translational Science 2.5:561-574;
TWPKHFDKHTFYSILKLGKH-OH (SEQ ID NO:3), Sakamoto, et al., 2017, Biochemistry and biophysics reports 12:135-139;
Angiopep-2: TFFYGGSRGKRNNFKTEEY-OH (SEQ ID NO:4), Sakamoto, et al., 2017, Biochemistry and biophysics reports 12:135-139;
LRKLRKRLLRDADDLLRKLRKRLLRDADDL (SEQ ID NO:5), Croy, et al., 2004, Biochemistry 43.23:7328-7335;
TEELRVRLASHLRKLRKRLL (SEQ ID NO:6), Croy, et al., 2004, Biochemistry 43.23:7328-7335;
Rap12: EAKIEKHNHYQK (SEQ ID NO:7), Ruan, et al., 2018, Journal of Controlled Release 279:306-315;
Rap22: EAKIEKHNHYQKQLEIAHEKLR (SEQ ID NO:8), Ruan, et al., 2018, Journal of Controlled Release 279:306-315; or
ANG: TFFYGGSRGKRNNFKTEEY (SEQ ID NO:9) Kim, et al., 2016, Scientific reports 6:34297.

Low Density Lipoprotein Receptor (LDLR):

In certain embodiments, the CRBM is a LDLR (low density lipoprotein receptor) binding group comprising one of the following amino acid sequences:
VH4127: cM-Thz-RLRG-Pen (cyclized c-Pen) (SEQ ID NO:10), Molino, et al., 2017, The FASEB Journal 31.5:1807-1827;
VH434: CMPRLRGC (cyclized C-C) (SEQ ID NO:11), Molino, et al., 2017, The FASEB Journal 31.5:1807-1827;
VH101: HLDCMPRGCFRN (cyclized C-C) (SEQ ID NO:12), David, et al., 2018, PloS one 13.2: 0191052;
VH202: CQVKSMPRC (cyclized C-C) (SEQ ID NO:13), David, et al., 2018, PloS one 13.2: 0191052;
VH203: CTTPMPRLC (cyclized C-C) (SEQ ID NO:14), David, et al., 2018, PloS one 13.2: 0191052;
VH204: CKAPQMPRC (cyclized C-C) (SEQ ID NO:15), David, et al., 2018, PloS one 13.2: 0191052;
VH205: CLNPSMPRC (cyclized C-C) (SEQ ID NO:16), David, et al., 2018, PloS one 13.2: 0191052;
VH306: CLVSSMPRC (cyclized C-C) (SEQ ID NO:17), David, et al., 2018, PloS one 13.2: 0191052;
VH307: CLQPMPRLC (cyclized C-C) (SEQ ID NO:18), David, et al., 2018, PloS one 13.2: 0191052;
VH308: CPVSSMPRC (cyclized C-C) (SEQ ID NO:19), David, et al., 2018, PloS one 13.2: 0191052;
VH309: CQSPMPRLC (cyclized C-C) (SEQ ID NO:20), David, et al., 2018, PloS one 13.2: 0191052;
VH310: CLTPMPRLC (cyclized C-C) (SEQ ID NO:21), David, et al., 2018, PloS one 13.2: 0191052;
VH411: DSGLCMPRLRGCDPR (cyclized C-C) (SEQ ID NO:22), David, et al., 2018, PloS one 13.2: 0191052;
VH549: TPSAHAMALQSLSVG (SEQ ID NO:23), David, et al., 2018, PloS one 13.2: 0191052;
AcVH411: Ac-DSGLCMPRLRGCDPR-NH₂(cyclized C-C) (SEQ ID NO:24), David, et al., 2018, PloS one 13.2: 0191052;
PrVH434: Pr-CMPRLRGC-NH2(cyclized C-C) (SEQ ID NO:25), David, et al., 2018, PloS one 13.2: 0191052;
VH445: Pr-cMPRLRGC-NH2 (cyclized C-C) (SEQ ID NO:26), David, et al., 2018, PloS one 13.2: 0191052;
VH4127: Pr-cMThzRLRG-Pen-NH₂ (cyclized C-Pen) (SEQ ID NO:27), David, et al., 2018, PloS one 13.2: 0191052;
AcVH434: Ac-CMPRLGC-NH₂(cyclized C-C) (SEQ ID NO:28), Jacquot, et al., 2016, Molecular pharmaceutics 13.12:4094-4105;
AcVH445: Ac-cMPRLRGC-NH2 (cyclized C-C) (SEQ ID NO:29), Jacquot, et al., 2016, Molecular pharmaceutics 13.12:4094-4105;
VH4106: Ac-D-Pen-M-Thz-RLRGC-NH₂ (cyclized Pen-C) (SEQ ID NO:30), Jacquot, et al., 2016, Molecular pharmaceutics 13.12:4094-4105;
VH4127: Pr-cM-Thz-RLRG-Pen-NH₂ (cyclized c-Pen) (SEQ ID NO:31), Jacquot, et al., 2016, Molecular pharmaceutics 13.12:4094-4105;
VH4128: Pr-cM-Thz-RLR-Sar-Pen-NH₂ (cyclized C-Pen) (SEQ ID NO:32), Jacquot, et al., 2016, Molecular pharmaceutics 13.12:4094-4105;
VH4129: Pr-cM-Pip-RLR-Sar-C—NH2 (cyclized C-C) (SEQ ID NO:33), Jacquot, et al., 2016, Molecular pharmaceutics 13.12:4094-4105;
VH4130: Pr-cM-Pip-RLRG-Pen-NH₂ (cyclized c-Pen) (SEQ ID NO:34), Jacquot, et al., 2016, Molecular pharmaceutics 13.12:4094-4105; or
VH4131: Pr-cM-Pip-RLR-Sar-Pen-NH₂ (cyclized c-Pen) (SEQ ID NO:35), Jacquot, et al., 2016, Molecular pharmaceutics 13.12:4094-4105.

FcγRI Receptor:

In certain embodiments, the CRBM is a FcγRI binding group comprising one of the following amino acid sequences:
Cp22: TDT C LMLPLLLG C DEE (cyclized C-C) (SEQ ID NO:36), Bonetto, et al, 2009, The FASEB Journal 23.2:575-585;
Cp21: DPI C WYFPRLLG C TTL (cyclized C-C) (SEQ ID NO:37), Bonetto, et al, 2009, The FASEB Journal 23.2:575-585;
Cp23: WYP C YIYPRLLG C DGD (cyclized C-C) (SEQ ID NO:38), Bonetto, et al, 2009, The FASEB Journal 23.2:575-585;
Cp24: GNI C MLIPGLLG C SYE (cyclized C-C) (SEQ ID NO:39), Bonetto, et al, 2009, The FASEB Journal 23.2:575-585;
Cp33: VNS C LLLPNLLG C GDD (cyclized C-C) (SEQ ID NO:40), Bonetto, et al, 2009, The FASEB Journal 23.2:575-585;
Cp25: TPV C ILLPSLLG C DTQ (cyclized C-C) (SEQ ID NO:41), Bonetto et al, 2009, The FASEB Journal 23.2:575-585;
Cp26: TVL C SLWPELLG C PPE (cyclized C-C) (SEQ ID NO:42), Bonetto, et al, 2009, The FASEB Journal 23.2:575-585;
Cp27: TFS C LMWPWLLG C ESL (cyclized C-C) (SEQ ID NO:43), Bonetto, et al, 2009, The FASEB Journal 23.2:575-585;
Cp32: FGT C YTWPWLLG C EGF (cyclized C-C) (SEQ ID NO:44), Bonetto, et al, 2009, The FASEB Journal 23.2:575-585;
Cp34: SLF C RLLLTPVG C VSQ (cyclized C-C) (SEQ ID NO:45), Bonetto, et al, 2009, The FASEB Journal 23.2:575-585;
P35: HLL V LPRGLLG C TTLA (cyclized C-C) (SEQ ID NO:46), Bonetto, et al, 2009, The FAST I3 Journal 23.2:575-585;
Cp28: TSL C SMFPDLLG C FNL (cyclized C-C) (SEQ ID NO:47), Bonetto, et al, 2009, The FASEB Journal 23.2:575-585;
Cp29: SHP C GRLPMLLG C AES (cyclized C-C) (SEQ ID NO:48), Bonetto, et al, 2009, The FASEB Journal 23.2:575-585;
P37: TST C SMVPGPLGAV STW (cyclized C-C) (SEQ ID NO:49), Bonetto, et al, 2009, The FASEB Journal 23.2:575-585;
Cp30: KDP C TRWAMLLG C DGE (cyclized C-C) (SEQ ID NO:50), Bonetto, et al, 2009, The FASEB Journal 23.2:575-585;
Cp31: IMT C SVYPFLLG C VDK (cyclized C-C) (SEQ ID NO:51), Bonetto, et al, 2009, The FASEB Journal 23.2:575-585;
Cp36: IHS C AHVMRLLG C WSR (cyclized C-C) (SEQ ID NO:52), Bonetto, et al, 2009, The FASEB Journal 23.2:575-585.

Transferrin Receptor:

In certain embodiments, the CRBM is a transferrin receptor binding group comprising one of the following amino acid sequences:
Tf1: CGGGPFWWWP (SEQ ID NO:53), Sand, et al., 2016, Bioconjugate chemistry 28.2:471-480;
Tf2: CGGGHKYLRW (SEQ ID NO:54), San i, et al., 2016, Bioconjugate chemistry 28.2:471-480;
Tf3: CGGGKRIFMV (SEQ ID NO:55), Sand, et al., 2016, Bioconjugate chemistry 28.2:471-480;
Tf2-scr: CGGGKWHYLR (SEQ ID NO:56), Sand, et al., 2016, Bioconjugate chemistry 28.2:471-480;
TfR-Ti2: THRPPMWSPVWP (SEQ ID NO:57), Mu, et al., 2017, Scientific reports 7.1:3487;
HAIYPRH (SEQ ID NO:67), Lee, et al, 2001, European journal of biochemistry 268.7:2004-2012);
THRPPMWSPVWP (SEQ ID NO:58), Lee, et al, 2001, European jour of biochemistry 268.7:2004-2012);
THRPPMWSPVWP (SEQ ID NO:59), Wangler, et al., 2011, Molecular Imaging and Biology 13.2:332-341.

Macrophage Scavenger Receptor:

In certain embodiments, the CRBM is a macrophage scavenger receptor binding moiety comprising one of the following amino acid sequences:
PP1: LSLERFLRCWSDAPA (SEQ ID NO:60), Segers, et al., 2012, Arteriosclerosis, thrombosis, and vascular biology 32.4:971-978;
PP1-13: LERFLRCWSDAPA (SEQ ID NO:61), Segers, et al., 2012, Arteriosclerosis, thrombosis, and vascular biology 32.4:971-978;
PP1-11: RFLRCWSDAPA (SEQ ID NO:62), Segers, et al., 2012, Arteriosclerosis, thrombosis, and vascular biology 32.4:971-978;
PP1-9: LRCWSDAPA (SEQ ID NO:63), Segers, et al., 2012, Arteriosclerosis, thrombosis, and vascular biology 32.4:971-978;
PP1-7: CWSDAPA (SEQ ID NO:64), Segers, et al., 2012, Arteriosclerosis, thrombosis, and vascular biology 32.4:971-978;
4F: DWFKAFYDKVAEKFKEAF (SEQ ID NO:65), Neyen, et al., 2009, Biochemistry 48.50:11858-11871);

As used herein, Pen is Penicillamine, Thz is thiazolidine-4-carboxylic acid, Sar is sarcosine, Pip is pipecolic acid, Nleu is norleucine, and NMeLeu is N-methylleucine.

G-Protein Coupled Receptor:

In certain embodiments, the CRBM is a G-protein coupled receptor (GPCR) binding moiety. In certain embodiments, the binding moiety binds to the GPCR and induces receptor internalization. In certain embodiments, the receptor is CXCR7 (see, for example, Nalawansha, et al., 2019, ACS Cent. Sci. 5(6):1079-1084). In certain embodiments, the binding moiety comprises the following:

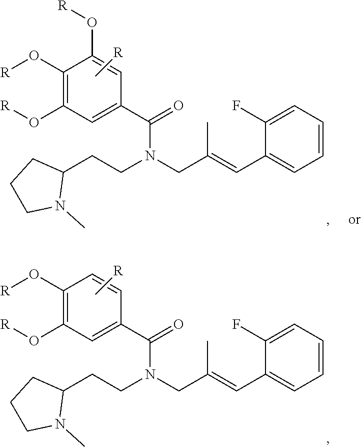

wherein each occurrence of R is independently H or C₁-C₆ alkyl. In certain embodiments, the CRBM can be attached to the compound of the disclosure (such as but not limited to the REAG) using one of the following reagents (which may be optionally protected with appropriately protecting groups):

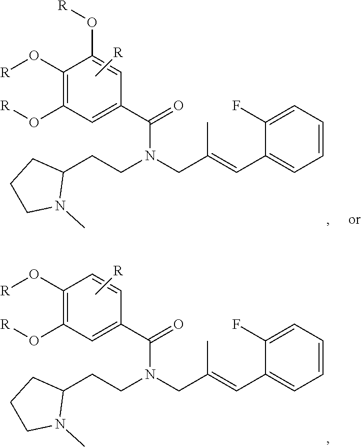

wherein at least one occurrence of R is REAG, and wherein the remaining occurrences of R are independently H or C₁-C₆ alkyl.

Asialoglycoprotein Receptor (ASGPR):

The disclosure contemplates the use of a ASGPR binding moiety (ASGPRBM).

In certain embodiments, the ASGPRBM group is any such group recited in Huang, et al., 2017, Bioconjugate Chem. 28:283-295, which is incorporated herein in its entirety by reference.

In certain embodiments, the ASGPRBM group comprises the structure:

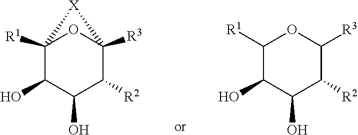

wherein X is a linker of 1-4 atoms in length and comprises O, S, N(R^{N1}), or C(R^{N1})(R^{N1}) groups, such that:
when X is a linker of 1 atom in length, X is O, S, N(R^{N1}), or C(R^{N1})(R^{N1}),
when X is a linker of 2 atoms in length, no more than 1 atom of X is O, S, or N(R^{N1}),
when X is a linker of 3 or 4 atoms in length, no more than 2 atoms of X are independently O, S, or N(R^{N1}).

In certain embodiments, each occurrence of R^{N1} is independently H or C₁-C₃ alkyl optionally substituted with 1-3 independently selected halogens and/or 1-2 hydroxyl groups.

In certain embodiments, the X in ASGPRBM is —O—C(R^{N1})(R^{N1})—, —C(R^{N1})(R^{N1})—O—, —S—C(R^{N1})(R^{N1})—, —C(R^{N1})(R^{N1})—S—, —N(R^{N1})—C(R^{N1})(R^{N1})—, —C(R^{N1})(R^{N1})—N(R^{N1})—, or —C(R^{N1})(R^{N1})—C(R^{N1})(R^{N1})—, when X is 2 atoms in length.

In certain embodiments, the X in ASGPRBM is —O—C(R^{N1})(R^{N1})—C(R^{N1})(R^{N1})—, —C(R^{N1})(R^{N1})—O—C(R^{N1})(R^{N1})—, —O—C(R^{N1})(R^{N1})—O—, —O—C(R^{N1})(R^{N1})—S—, —O—C(R^{N1})(R^{N1})—N(R^{N1})—, —S—C(R^{N1})(R^{N1})—C(R^{N1})(R^{N1})—, —C(R^{N1})(R^{N1})—S—C(R^{N1})(R^{N1})—, —C(R^{N1})(R^{N1})—C(R^{N1})(R^{N1})—S, —S—C(R^{N1})(R^{N1})—S—, —S—C(R^{N1})(R^{N1})—O—, —S—C(R^{N1})(R^{N1})—N(R^{N1})—, —N(R^{N1})—C(R^{N1})(R^{N1})—C(R^{N1})(R^{N1})—, —C(R^{N1})(R^{N1})—N(R^{N1})—C(R^{N1})(R^{N1})—, —C(R^{N1})(R^{N1})—C(R^{N1})(R^{N1})—N(R^{N1})—, —N(R^{N1})—C(R^{N1})(R^{N1})—N(R^{N1})—, or —C(R^{N1})(R^{N1})—C(R^{N1})(R^{N1})— C(R^{N1})(R^{N1}), when X is 3 atoms in length.

In certain embodiments, the X in ASGPRBM is —O—C(R^{N1})(R^{N1})—C(R^{N1})(R^{N1})—C(R^{N1})(R^{N1})—, —C(R^{N1})(R^{N1})—O—C(R^{N1})(R^{N1})—C(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})—, —C(R^{N1})(R^{N1})—S—C(R^{N1})(R^{N1})—C(R^{N1})(R^{N1})—, —C(R^{N1})(R^{N1})—C(R^{N1})(R^{N1})—S—C(R^{N1})(R^{N1})—, —S—C(R^{N1})(R^{N1})—S—C—(R^{N1})(R^{N1})—, —N(R^{N1})—C(R^{N1})(R^{N1})—C(R^{N1})(R^{N1})—C(R^{N1})(R^{N1})—, or —C(R^{N1})(R^{N1})—N(R^{N1})—C(R^{N1})(R^{N1})—C(R^{N1})(R^{N1})—, when X is 4 atoms in length.

In certain embodiments, X is OCH₂ and R^{N1} is H.

In certain embodiments, X is CH₂O and R^{N1} is H.

In certain embodiments, the ASGPRBM comprises the structure:

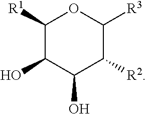

In certain embodiments, the ASGPRBM comprises the structure:

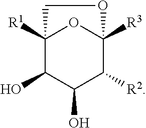

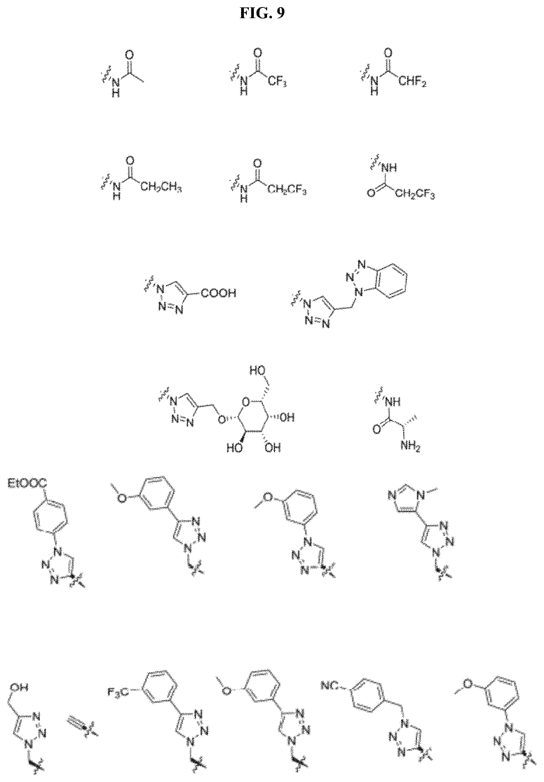

In certain embodiments, R¹ is a group illustrated in FIG. 9. In certain embodiments, R³ is a group illustrated in FIG. 9. In certain embodiments, R¹ and R³ are each independently a group illustrated in FIG. 9.

In certain embodiments, R¹ and R³ are each independently H, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}(vinyl), —O(CH₂)_{K}(vinyl), —(CH₂)_{K}(alkynyl), —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens.

In certain embodiments, R¹ and R³ are each independently Ph(CH₂)_{K}—, which is optionally substituted with: 1-3 independently selected halogens; C₁-C₄ alkyl optionally substituted with 1-3 independently selected halogens and/or 1-2 hydroxyl groups; or C₁-C₄ alkoxy optionally substituted with 1-3 independently selected halogens and/or 1-2 hydroxyl groups.

In certain embodiments, R¹ and R³ are each independently a group of structure

—O—(CH₂)_{K}—CH(OH)—(CH₂)K′—R⁷,

wherein R⁷ is: C₁-C₄ alkoxy optionally substituted with 1-3 independently selected halogens and/or 1-2 hydroxy groups; —NR^{N3}R^{N4}; or —(CH₂)_{K′}—O—(CH₂)_{K}—CH₂—CH═CH₂.

In certain embodiments, K is 0. In certain embodiments, K is 1. In certain embodiments, K is 2. In certain embodiments, K is 3. In certain embodiments, K is 4.

In certain embodiments, K′ is 1. In certain embodiments, K′ is 2. In certain embodiments, K′ is 3. In certain embodiments, K′ is 4.

In certain embodiments, each occurrence of R^{N3} is independently H or C₁-C₃ alkyl optionally substituted with 1-3 independently selected halogens and/or 1-2 hydroxyl groups;

In certain embodiments, each occurrence of R^{N4} is independently H, C₁-C₃ alkyl optionally substituted with 1-3 independently selected halogens and/or 1-2 hydroxyl groups, or Ph-(CH₂)_{K}—.

In certain embodiments, R¹ and R³ are each independently selected from the group consisting of:
—(CH₂)_{K}OH,

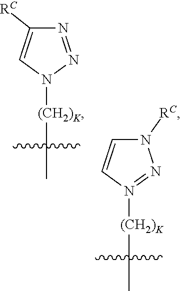

L¹—≡—, L¹—(CH₂)_{K}—, and CYC—(CH₂)_{K}—, wherein CYC is selected from the group consisting of:

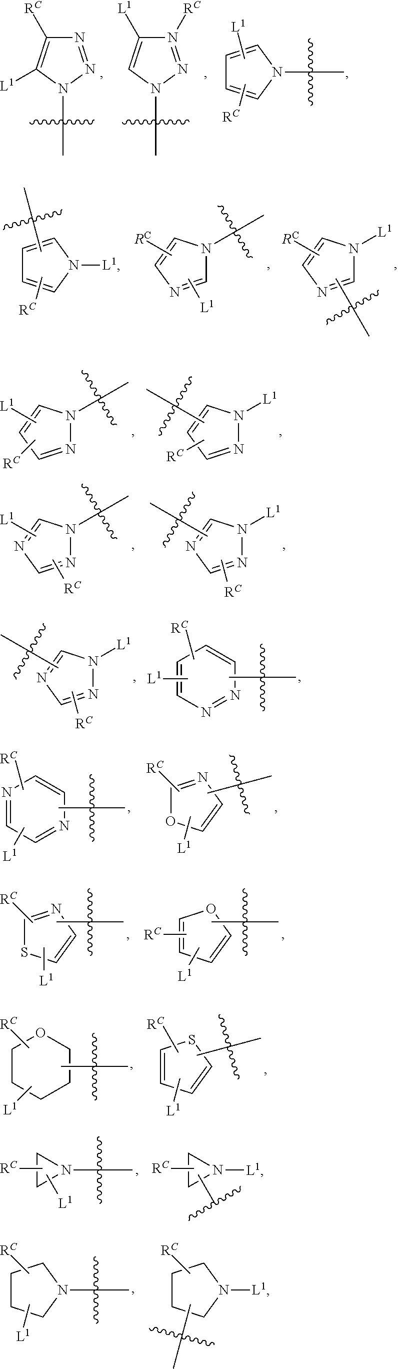

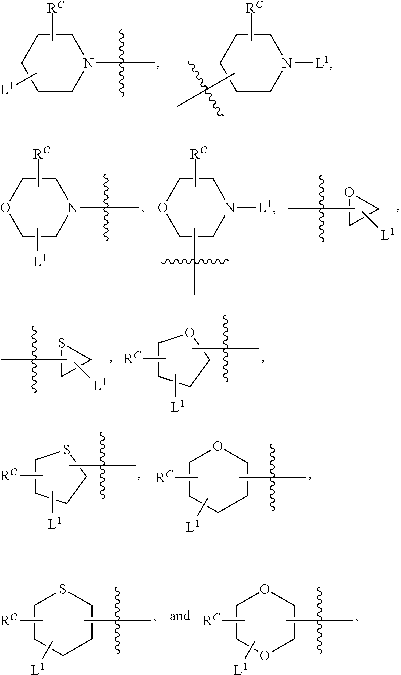

wherein the bond marked with [symbol] indicating the site on CYC whereto —(CH₂)_{K} is connected.

In certain embodiments, L¹ is a bond, -Linker, —CON-Linker, or —CON-Linker-CON.

In certain embodiments, R^{C} is absent, H, C₁-C₄ alkyl optionally substituted with 1-3 optionally substituted halogens and/or 1-2 hydroxyl groups, or a group of structure:

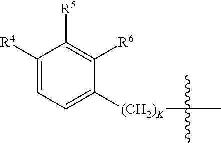

wherein R⁴, R⁵, and R⁶ are each independently H, F, Cl, Br, I, CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens, C₁-C₃-alkoxy optionally substituted with 1-3 independently selected halogens, —(CH₂)_{K}COOH, —(CH₂)_{K}C(═O)O—(C₁-C₄ alkyl) optionally substituted with 1-3 independently selected halogens, O—C(═O)—(C₁-C₄ alkyl) optionally substituted with 1-3 independently selected halogens, or —C(═O)—(C₁-C₄ alkyl) optionally substituted with 1-3 independently selected halogens.

In certain embodiments, each occurrence of R^{N2} is independently H or C₁-C₃ alkyl optionally substituted with 1-3 independently selected halogens and/or 1-2 hydroxyl groups.

In certain embodiments, R^{C} is

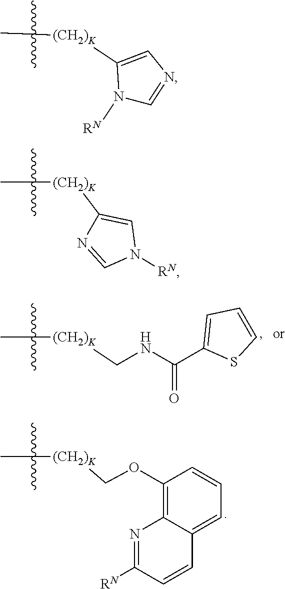

In certain embodiments, R¹ and R³ are each independently (C₃-C₈ saturated carbocyclic) —(CH₂)_{K}—, wherein the carbocyclic is further substituted with —L¹ and —R^{C}.

In certain embodiments, each occurrence of R^{N} is independently H or C₁-C₃ alkyl optionally substituted with 1-3 independently selected halogens and/or 1-2 hydroxyl groups.

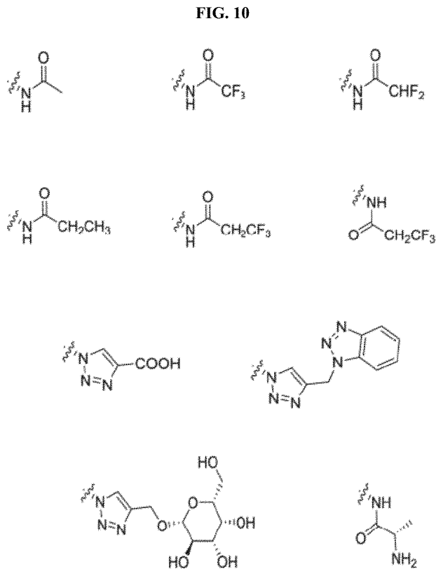

In certain embodiments, R² is a group illustrated in FIG. 10.

In certain embodiments, R² is —(CH₂)_{K}—N(R^{N1})—C(═O)R^{AM}.

In certain embodiments, R^{AM} is H, C₁-C₄ alkyl optionally substituted with 1-3 independently selected halogens and/or 1-2 hydroxyl groups, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, —C(═O)(C₁-C₄ alkyl) optionally substituted with 1-3 independently selected halogens, or —(CH₂)_{K}—NR^{N3}R^{N4}.

In certain embodiments, R² is

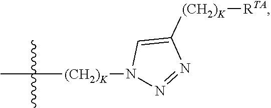

wherein:
R^{TA} is H, CN, NR^{N1}R^{N2}, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens,
or
R^{TA} is C₃-C₁₀ aryl or a 3- to 10-membered heteroaryl group containing 1-5 non-carbon ring atoms, each of the aryl or heteroaryl groups being optionally substituted with 1-3 groups independently selected from CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens and/or 1-2 hydroxyl groups, —(C₁-C₃-alkoxy) optionally substituted from 1-3 independently selected halogens, —(CH₂)_{K}COOH, —(CH₂)_{K}C(═O)O—(C₁-C₄ alkyl) optionally substituted with 1-3 independently selected halogens, —OC(═O)(C₁-C₄ alkyl) optionally substituted with 1-3 independently selected halogens, or —(CH₂)_{K}C(═O)—(C₁-C₄ alkyl) optionally substituted with 1-3 independently selected halogens,
or
R^{TA} is

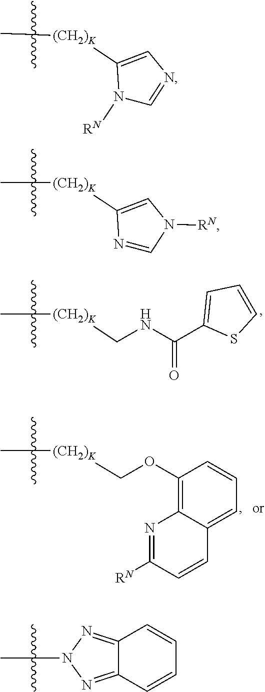

optionally substituted with 1-3 C₁-C₃ alkyl groups optionally substituted with 1-3 independently selected halogens, or

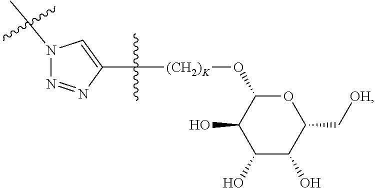

R^{TA} is wherein each —(CH₂)_{K} group is optionally substituted with 1-4 C₁-C₃ alkyl groups optionally substituted with 1-3 fluoro groups or 1-2 hydroxyl groups.

In certain embodiments, the ASGPRBM group comprises the structure:

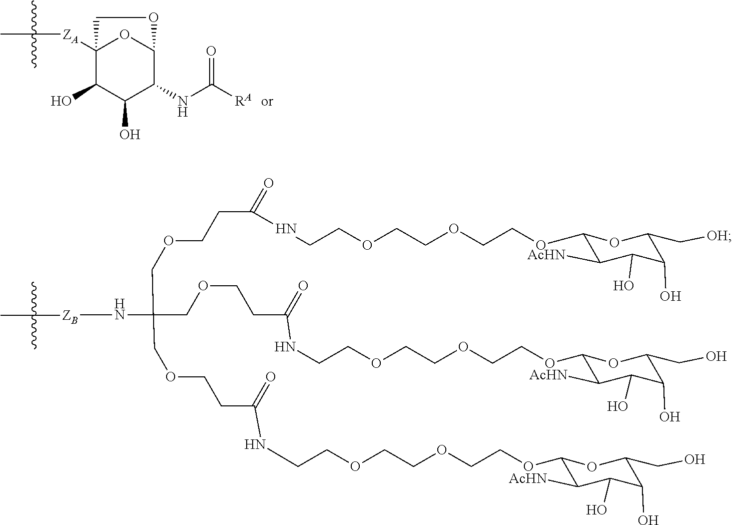

wherein:
R^{A} is C₁-C₃ alkyl optionally substituted with 1-5 independently selected halogens;
Z_{A} is —(CH₂)_{IM}—, —O—(CH₂)_{IM}—, —S—(CH₂)_{IM}—, —NR^{M}—(CH₂)_{IM}—, —C(═O)—(CH₂)_{IM}—, a PEG group containing from 1 to 8 ethylene glycol residues, or —C(O)(CH₂)_{IM}NR^{M}—;
Z_{B} is absent, —(CH₂)_{IM}—, —C(═O)—(CH₂)_{IM}—, or —C(═O)(CH₂)_{IM}—NR^{M}—;
R^{M} is H or C₁-C₃ alkyl optionally substituted with 1-2 hydroxyl groups; and
each occurrence of IM is independently 0, 1, 2, 3, 4, 5, or 6.

In certain embodiments, R^{A} is methyl or ethyl, either of which is optionally substituted with 1-3 fluorines.

In certain embodiments, Z_{A} is a PEG group containing from 1 to 4 ethylene glycol residues.

In certain embodiments, the ASGPRBM group comprises one of the following (Mamidyala, et al., 2012, J. Am. Chem. Soc. 134:1978-1981):

3

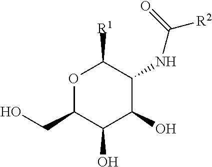

4

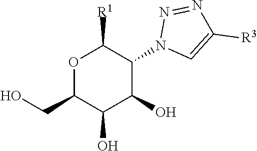

| | R¹ | # | R² | # | R² | # | R³ | # | R³ | # | R³ |
|---|---|---|---|---|---|---|---|---|---|---|---|
| a | Me | 1 | Me | 9 | CH₂NH₂ | 1 | Ph | 10 | 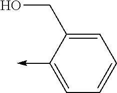 | 14 | 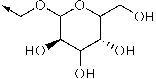 |
| b | 4- OMePh | 2 | [symbol] | 10 | CH₂CF₃ | 2 | 4-OMePh | 11 | 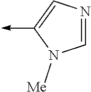 | 15 | CH₂OH |
| | | 3 | i-Pr | 11 | 2-furyl | 3 | 3-OMePh | 12 | 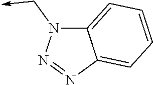 | 16 | CH₂NH₂ |
| | | 4 | t-Bu | 12 | Ph | 4 | 3-NH₂Ph | 13 | 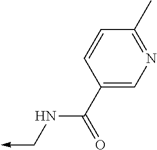 | 17 | CH₂NHMe |
| | | 5 | CCl₂ | 13 | 4-OMePh | 5 | 4-NMe₂Ph | | | 18 | CH₂NMe₂ |
| | | 6 | CF₃ | 14 | 3-OMePh | 6 | 2-pyridyl | | | 19 | CO₂H |
| | | 7 | Ot-Bu | 15 | 4-CNPh | 7 | 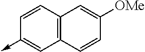 | | | 20 | CH₂NHCOPh |
| | | 8 | CH₂CO₂H | 16 | 3-pyridyl | 8 | 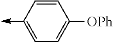 | | | 21 | CH₂NHCOMe |
| | | | | 17 | 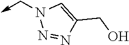 | 9 | 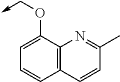 | | | | |

| | | R¹ | | R¹ |
|---|---|---|---|---|
| 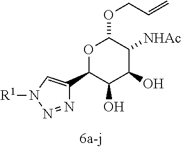 | a | Me | g | 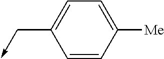 |
| | b | 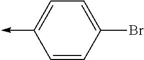 | h | 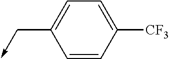 |
| | c | 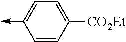 | i | 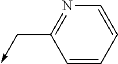 |
| | d | 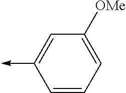 | j | 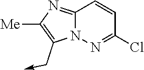 |
| | e | 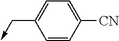 | | |
| | f | 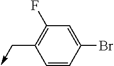 | | |
| | | R² | | R² |
| 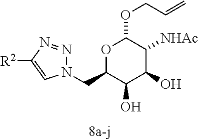 | a | CH₂OH | g | 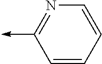 |
| | b | 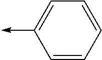 | h | 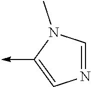 |
| | c | 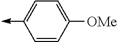 | i | 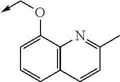 |
| | d | 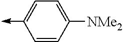 | j | 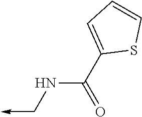 |
| | e | 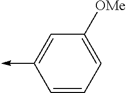 | | |
| | f | 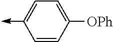 | | |

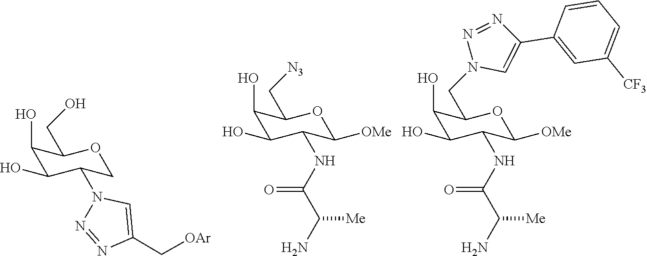

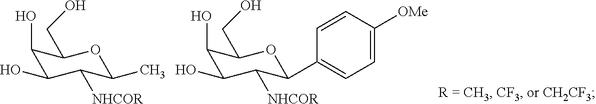

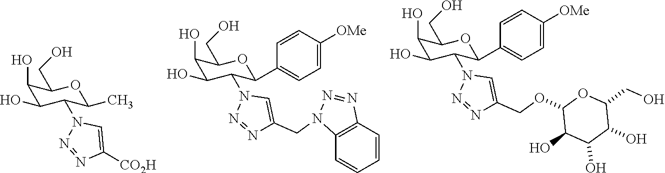

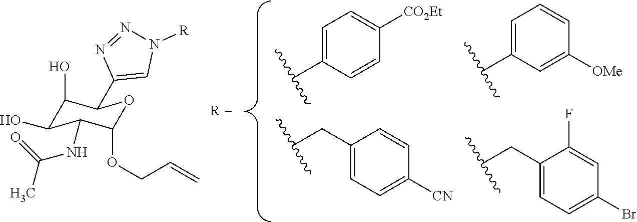

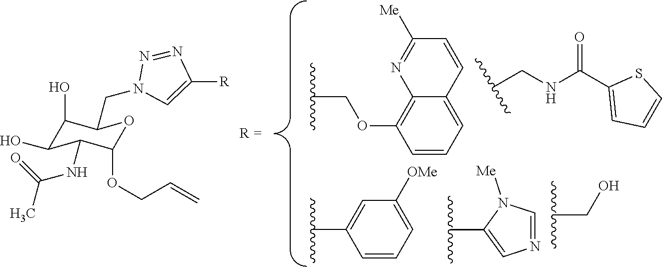

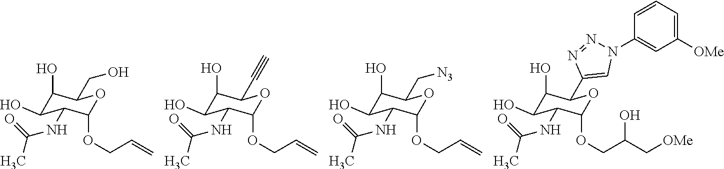

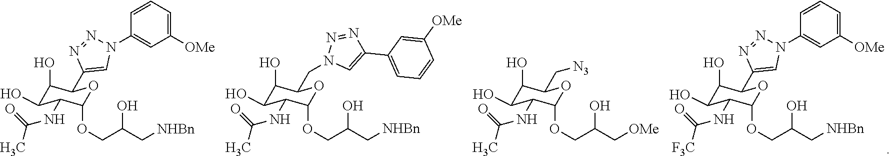

[symbol]

indicates data missing or illegible when filed

In certain embodiments, the ASGPRBM group comprises one of the following (Sanhueza, et al., 2017, J. Am. Chem. Soc. 139:3528-3536):

| 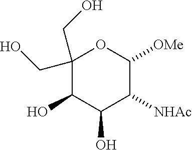 [SMILES: CO[C@H]1OC(CO)(CO)[C@H](O)[C@H](O)[C@H]1NC(C)=O] | |
|---|---|
| 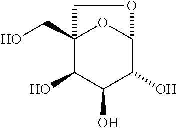 [SMILES: OC[C@@]12CO[C@@H](O1)[C@H](O)[C@@H](O)[C@H]2O] | |
| 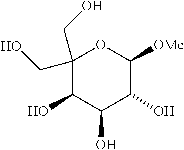 [SMILES: CO[C@@H]1OC(CO)(CO)[C@H](O)[C@H](O)[C@H]1O] | |
| 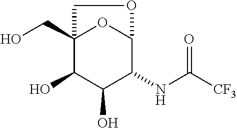 [SMILES: O=C(N[C@H]1[C@H]2OC[C@](CO)(O2)[C@H](O)[C@@H]1O)C(F)(F)F] | |
| 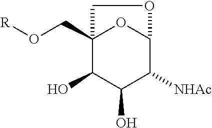 | |
| | R |
| 16a | Et |
| 16b | n-C₃H₇ |
| 16c | n-C₄H₉ |
| 16d | n-C₅H₁₁ |
| 16e | n-C₆H₁₃ |
| 16f | (CH₂CH₂O)₄Me |

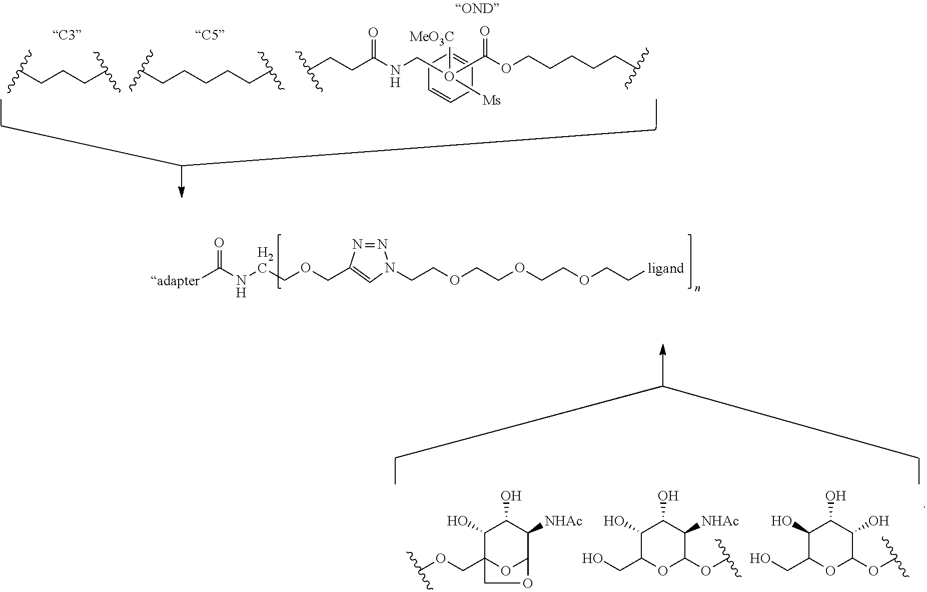

### Linker & CON

In certain embodiments, the Linker is a polyethylene glycol containing linker having 1-12 ethylene glycol residues.

In certain embodiments, the Linker comprises the structure: —CH₂CH₂(OCH₂CH₂)ₘOCH₂—, —(CH₂)ₘCH₂—, —[N(R^{a})—CH(R^{b})(C═O)]ₘ—, or a polypropylene glycol or polypropylene-co-polyethylene glycol group containing 1-100 alkylene glycol units;
wherein each R^{a} is independently H, C₁-C₃ alkyl, or C₁-C₆ alkanol, or combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
wherein m is an integer ranging from 1 to 15.

In certain embodiments, the Linker comprises the structure —[N(R′—(CH₂)₁₋₁₅—C(═O)]—, wherein R′ is H or a C₁-C₃ alkyl optionally substituted with 1-2 hydroxyl groups, and m is an integer ranging from 1 to 100.

In certain embodiments, the Linker comprises the structure

—Z—D—Z′—,

wherein:
Z and Z′ are each independently a bond, —(CH₂)ᵢ—O—, —(CH₂)ᵢ—S—, —(CH₂)ᵢ—N(R)—,

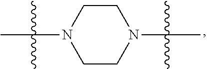 [SMILES: *N1CCN(*)CC1]

—(CH₂)ᵢ—C(R²)═C(R²)— (cis or trans), —(CH₂)ᵢ—≡—, or —Y—C(═O)—Y—;
each R is independently H, C₁-C₃ alkyl, or C₁-C₆ alkanol;
each R² is independently H or C₁-C₃ alkyl;
each Y is independently a bond, O, S, or N(R);
each i is independently 0 to 100; in certain embodiments 0 to 75; in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 0, 1, 2, 3, 4 or 5;
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X¹ is O, S, or N(R);
j is an integer ranging from 1 to 100; in certain embodiments 1 to 75;
in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 1, 2, 3, 4 or 5;
m′ is an integer ranging from 1 to 100; in certain embodiments 1 to 75; in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 1, 2, 3, 4 or 5;
n is an integer ranging from 1 to 100; in certain embodiments 1 to 75; in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 1, 2, 3, 4 or 5.

In certain embodiments, the Linker comprises a structure:

—CH₂—(OCH₂CH₂)ₙ—CH₂—, —(CH₂CH₂O)_{n′}CH₂CH₂—, or —(CH₂CH₂CH₂O)ₙ—,

wherein each n and n′ is independently an integer ranging from 1 to 25; in certain embodiments 1 to 15; in certain embodiments 1 to 12; in certain embodiments 2 to 11; in certain embodiments 2 to 10; in certain embodiments 2 to 8; in certain embodiments 2 to 6; in certain embodiments 2 to 5; in certain embodiments 2 to 4; in certain embodiments 2 or 3; in certain embodiments 1, 2, 3, 4, 5, 6, 7, or 8.

In certain embodiments, the Linker comprises a structure:

-PEG-CON-PEG-

wherein each PEG is independently a polyethylene glycol group containing from 1-12ethylene glycol residues and CON is a triazole group

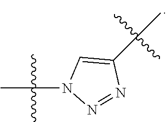 [SMILES: *c1cn(*)nn1]

In certain embodiments, the CON comprises a structure:

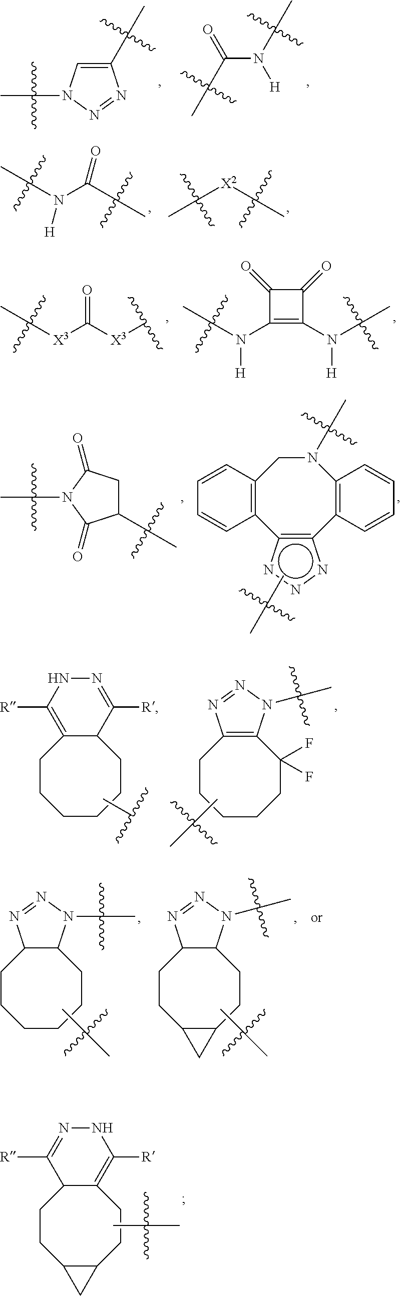

wherein R′ and R″ are each independently H, methyl, or a bond.

In certain embodiments, the CON comprises a diamide structure:

—C(═O)—N(R¹)—(CH₂)_{n″}—N(R¹)C(═O)—,

—N(R¹)—C(═O)(CH₂)_{n″}—C(═O)N(R¹)—, or

—N(R¹)—C(═O)(CH₂)_{n″}—N(R¹)C(═O)—;

wherein each R¹ is independently H or C₁-C₃ alkyl, and n″ is independently an integer from 0 to 8, in certain embodiments 1 to 7, in certain embodiments 1, 2, 3, 4, 5 or 6.

In certain embodiments, the CON comprises a structure:

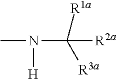

wherein:
R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}—, or —(CH₂)_{M2}O—(CH₂)_{M1}—C(O)NR⁴—, with the proviso that R^{1a}, R^{2a} and R^{3a} are not simultaneously H;
each M1 is independently 1, 2, 3, or 4; in certain embodiments, 1 or 2;
each M2 is independently 0, 1, 2, 3, or 4; in certain embodiments, 0, 1 or 2;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0.

In certain embodiments, the CON comprises a structure:

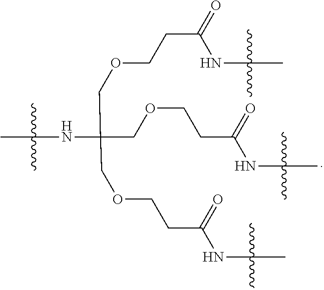 [SMILES: *NC(=O)CCOCC(COCCC(=O)N*)(COCCC(=O)N*)N*]

### Ab

Any antibody (Ab) that binds to an extracellular protein is useful within the present disclosure. In certain non-limiting embodiments, the antibody is a monoclonal antibody.

Non-limiting examples of extracellular proteins contemplated within the present disclosure include 1-40-β-amyloid, 5′-nucleotidase, activated F9, F10, activin receptor-like kinase 1, alpha-fetoprotein, amyloid, angiopoietin 2, angiopoietin 3, anthrax toxin, AOC3, AOC3 (VAP-1), Bacillus anthracis anthrax, BAFF, beta amyloid, c-Met, C1s, C242 antigen, C5, CA-125, calcitonin, calcitonin gene-related peptide, calcitonin gene-related peptide alpha, Canis lupus familiaris IL31, carbonic anhydrase 9 (CA-IX), CEA, CEA-related antigen, CEACAM5, CFD, CGRP, clumping factor A, coagulation factor III, complement C5a, CSF1, MCSF, CSF2, dabigatran, E. coli shiga toxin type-1, E. coli shiga toxin type-2, EGFL7, endotoxin, episialin, FGF 23, fibrin II, beta chain, fibronectin extra domain-B, folate hydrolase, GDF-8, gelatinase B, GMCSF, growth differentiation factor 8, hemagglutinin, hemagglutinin HA, HGF, HIV-1, HNGF, Hsp90, human beta-amyloid, human scatter factor receptor kinase, human TNF, IFN-α, IFN-γ, IgE, IgE Fc region, IGF1, IGF2, IGHE, IL 17A, IL 17A and IL 17F, IL 20, IL-1, IL-12, IL-23, IL-13, IL-17, IL-10, IL-22, IL-4, IL-5, IL-6, IL17A and IL17F, IL1A, IL2, IL23, IL23A, IL31RA, IL6, IL6R, IL9, ILGF2, Influenza A hemagglutinin, influenza A virus hemagglutinin, influenza A virus hemagglutinin HA, interferon gamma, interferon gamma-induced protein, interleukin 1 alpha, interleukin 13, interleukin 17 alpha, interleukin 17 alpha, TNF, interleukin 17A, kallikrein, LOXL2, LRRC15, LTA, MASP-2, MCP-1, MIF, MST1R (aka RON), MUC1, myostatin, NACP, NCA-90 (granulocyte antigen), neural apoptosis-regulated proteinase 1, NGF, NOGO-A, Notch 1, NRP1, oxLDL, PCSK9, PD-L1, phosphatidylserine, RANKL, RGMA, root plate-specific spondin 3, RTN4, sclerostin, SDC1, serum amyloid A protein, serum amyloid P component, SOST, Staphylococcus aureus alpha toxin, tau protein, TFPI, TGF beta 1, TGF beta 2, TGF-β, TNF-α, TROP-2, TSLP, VEGF-A, VEGF-A and Ang-2, VEGFA, and VWF.

Non-limiting examples of antibodies useful within the present disclosure include Abagovomab, Abrezekimab, Adalimumab, Aducanumab, Afasevikumab, Afelimomab, Alirocumab, Altumomab, Altumomab pentetate, Andecaliximab, Anrukinzumab, Arcitumomab, Ascrinvacumab, Atezolizumab, Atidortoxumab, Atinumab, Avelumab, Bapineuzumab, Bavituximab, Belimumab, Bermekimab, Besilesomab, Bevacizumab, Biciromab, Bimekizumab, Birtamimab, Blosozumab, Bococizumab, Brazikumab, Briakinumab, Brodalumab, Brolucizumab, Brontictuzumab, Burosumab, Cabiralizumab, Canakinumab, Cantuzumab, Cantuzumab ravtansine, Caplacizumab, Carlumab, Cergutuzumab, Cergutuzumab amunaleukin, Certolizumab, Certolizumab pegol, Cibisatamab, Clazakizumab, Clivatuzumab, Clivatuzumab tetraxetan, Concizumab, Crenezumab, Dectrekumab, Denosumab, Dezamizumab, Diridavumab, Domagrozumab, Dorlimomab, Dorlimomab aritox, Durvalumab, Dusigitumab, Eculizumab, Edobacomab, Efungumab, Eldelumab, Elezanumab, Elsilimomab, Emactuzumab, Emapalumab, Emicizumab, Enokizumab, Epitumomab, Epitumomab cituxetan, Eptinezumab, Erenumab, Evinacumab, Evolocumab, Faricimab, Fasinumab, Fezakinumab, Ficlatuzumab, Firivumab, Fletikumab, Fontolizumab, Fremanezumab, Fresolimumab, Frovocimab, Frunevetmab, Fulranumab, Galcanezumab, Gantenerumab, Gatipotuzumab, Gedivumab, Gevokizumab, Gimsilumab, Girentuximab, Golimumab, Gosuranemab, Guselkumab, Idarucizumab, Igovomab, Imalumab, Indatuximab, Indatuximab ravtansine, Infliximab, Istiratumab, Ixekizumab, Labetuzumab, Lacnotuzumab, Lampalizumab, Lanadelumab, Landogrozumab, Lebrikizumab, Lemalesomab, Lendalizumab, Lenzilumab, Lerdelimumab, Lesofavumab, Ligelizumab, Lodelcizumab, Lokivetmab, Lutikizumab, Marstacimab, Mepolizumab, Metelimumab, Mirikizumab, Nacolomab, Nacolomab tafenatox, Namilumab, Narnatumab, Navivumab, Naxitamab, Nebacumab, Nemolizumab, NEOD, Nerelimomab, Nesvacumab, Netakimab, Nofetumomab, Nofetumomab merpentan, Obiltoxaximab, Oleclumab, Olendalizumab, Olokizumab, Omalizumab, OMS, Onartuzumab, Oregovomab, Orticumab, Otilimab, Ozanezumab, Ozoralizumab, Parsatuzumab, Pascolizumab, Pasotuxizumab, Pateclizumab, Pemtumomab, Perakizumab, Pexelizumab, Placulumab, Ponezumab, Prasinezumab, Pritoxaximab, Quilizumab, Radretumab, Ralpancizumab, Ranevetmab, Ranibizumab, Ravulizumab, Raxibacumab, REGN-EB, Remtolumab, Reslizumab, Rilotumumab, Risankizumab, Romilkimab, Romosozumab, Rontalizumab, Rosmantuzumab, Sacituzumab, Sacituzumab govitecan, Samrotamab, Samrotamab vedotin, Sarilumab, Secukinumab, Setoxaximab, Setrusumab, Sifalimumab, Siltuximab, Simtuzumab, Sirukumab, Sofituzumab, Sofituzumab, Sofituzumab vedotin, Solanezumab, Sontuzumab, Stamulumab, Sulesomab, Sutimlimab, Suvizumab, Suvratoxumab, Tabalumab, Tacatuzumab, Tacatuzumab tetraxetan, Talizumab, Tanezumab, Tefibazumab, Telimomab, Telimomab aritox, Tesidolumab, Tezepelumab, Tibulizumab, Tildrakizumab, Timolumab, Tisotumab, Tisotumab vedotin, Tralokinumab, Trevogrumab, Urtoxazumab, Ustekinumab, Vanucizumab, Vapaliximab, Varisacumab, Vepalimomab, Vesencumab, Vobarilizumab, Vunakizumab, and Xentuzumab.

The antibody of interest can be incorporated within the compounds of the disclosure using any methods known in the art and/or any techniques described or illustrated herein. For example, the antibody can be attached to a Linker through a carboxylic acid group on the antibody's surface, using for example amide or ester formation chemistry. For example, the antibody can be attached to a Linker through an amine group on the antibody's surface, using for example amide formation chemistry. For example, the antibody can be attached to a Linker through a thiol group on the antibody's surface, using for example nucleophilic substitution chemistry. In that case, the surface cysteine residue can exist in the wild-type form of the Ab and/or can be introduced by mutation, using for example site-directed mutagenesis. The Linker useful within the disclosure can be any linker known in the art, as long as the presence of the linker does not significantly disturb the Ab's ability to bind to the circulating protein.

By way of a non-limited example, an antibody useful within the disclosure can bind to a circulating protein. As will be understood by one skilled in the art, any antibody that may recognize and specifically bind to a circulating protein is useful in the present disclosure. The disclosure should not be construed to be limited to any one type of antibody, either known or heretofore unknown, provided that the antibody can specifically bind to a circulating protein, and prevent or minimize biological activity of the circulating protein.

Methods of making and using such antibodies are well known in the art. For example, the generation of polyclonal antibodies may be accomplished by inoculating the desired animal with the antigen and isolating antibodies which specifically bind the antigen therefrom. Monoclonal antibodies directed against full length or peptide fragments of a protein or peptide may be prepared using any well-known monoclonal antibody preparation procedures, such as those described, for example, in Harlow et al. (1989, Antibodies, A Laboratory Manual, Cold Spring Harbor, N.Y.) and in Tuszynski et al. (1988, Blood 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. However, the disclosure should not be construed as being limited solely to methods and compositions including these antibodies, but should be construed to include other antibodies, as that term is defined elsewhere herein.

In some instances, it is desirable to prepare monoclonal antibodies from various mammalian hosts, such as rodents (e.g., mice), primates (e.g., humans), and so forth. Descriptions of techniques for preparing such monoclonal antibodies are well known and are described, for example, in Harlow et al., ANTIBODIES: A LABORATORY MANUAL, COLD SPRING HARBOR LABORATORY, Cold Spring Harbor, N.Y. (1988); Harlow et al., USING ANTIBODIES: A LABORATORY MANUAL, (Cold Spring Harbor Press, New York, 1998); Breitling et al., RECOMBINANT ANTIBODIES (Wiley-Spektrum, 1999); and Kohler et al., 1997 Nature 256: 495-497; and U.S. Pat. Nos. 5,693,762; 5,693,761; 5,585,089; and 6,180,370.

Nucleic acid encoding an antibody obtained using the procedures described herein may be cloned and sequenced using technology that is available in the art, and is described, for example, in Wright et al. (Critical Rev. Immunol. 1992, 12:125-168) and the references cited therein. Further, the antibody useful within the disclosure may be “humanized” using the technology described in Wright et al. (supra) and in the references cited therein, and in Gu et al. (Thrombosis and Hematocyst 1997, 77:755-759).

Alternatively, antibodies may be generated using phage display technology. To generate a phage antibody library, a cDNA library is first obtained from mRNA that is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, New York).

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell that expresses the corresponding antigen, the bacteriophage will bind to the cell. Bacteriophage that do not express the antibody will not bind to the cell. Such panning techniques are well known in the art and are described for example, in Wright et al. (Critical Rev. Immunol. 1992, 12:125-168).

Processes such as those described herein have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage that encode the Fab portion of an antibody molecule. However, the disclosure should not be construed to be limited solely to the generation of phage encoding Fab antibodies. Rather, phage that encode single chain antibodies (scFv/phage antibody libraries) are also included in the disclosure. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J Mol Biol 222:581-597). Panning of phage so generated for the isolation of a desired antibody is conducted in a manner similar to that described for phage libraries comprising Fab DNA.

The disclosure should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

Additional Galactose- and Talose-based ASGPR Binding Moieties

In one embodiment, the present invention is directed to compounds which are useful for removing circulating proteins which are associated with a disease state or condition in a patient or subject according to the general chemical structure of Formula II:

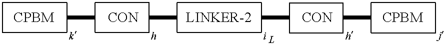

Formula II

The term “Extracellular Protein Targeting Ligand” as used herein is interchangeably used with the term CPBM (cellular protein binding moiety). The term “ASGPR Ligand” as used herein is interchangeably used with an asialoglycoprotein receptor (ASGPR) binding moiety as defined herein.

In the compound of Formula II, each [CON] is an optional connector chemical moiety which, when present, connects directly to [CPBM] or to [CRBM] or connects the [LINKER-2] to [CPBM] or to [CRBM].
In the compound of Formula II:
[LINKER-2] is a chemical moiety having a valency from 1 to 15 which covalently attaches to one or more [CRBM] and/or [CPBM] group, optionally through a [CON], including a [MULTICON] group, wherein said [LINKER-2] optionally itself contains one or more [CON] or [MULTICON] group(s);
k′ is an integer from 1 to 15;
j′ is an integer from 1 to 15;
h and h′ are each independently an integer from 0 to 15;
i_{L} is an integer from 0 to 15;
with the proviso that at least one of h, h′ and i_{L} is at least 1, or a pharmaceutically acceptable salt, stereoisomer, solvate or polymorph thereof.
A [MULTICON] group can connect one or more of a [CRBM] or [CPBM] to one or more of a [LINKER-2]. In various embodiments, [LINKER-2] has a valency of 1 to 10. In various embodiments, [LINKER-2] has a valency of 1 to 5. In various embodiments, [LINKER-2] has a valency of 1, 2 or 3. In various embodiments, in the compound of Formula II, the [LINKER-2] includes one or more of Linker^{A}, Linker^{B}, Linker^{C}, Linker^{D}, and/or combinations thereof as defined herein.

In the compound of Formula II, xx is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, yy is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, zz is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, X¹ is 1 to 5 contiguous atoms independently selected from O, S, N(R^{b}), and C(R⁴)(R⁴), wherein if X¹ is 1 atom then X¹ is O, S, N(R⁶), or C(R⁴)(R⁴), if X¹ is 2 atoms then no more than 1 atom of X¹ is O, S, or N(R⁶), if X¹ is 3, 4, or 5 atoms then no more than 2 atoms of X¹ are O, S, or N(R⁶);

R³ at each occurrence is independently selected from hydrogen, alkyl, heteroalkyl, haloalkyl (including —CF₃, —CHF₂, —CH₂F, —CH₂CF₃, —CH₂CH₂F, and —CF₂CF₃), arylalkyl, heteroarylalkyl, alkenyl, alkynyl, and, heteroaryl, heterocycle, —OR′, and —NR⁸R⁹;

R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷,

R⁶ and R⁷ are independently selected at each occurrence from hydrogen, heteroalkyl, alkyl, arylalkyl, heteroaryl alkyl, alkenyl, alkynyl, and, haloalkyl, heteroaryl, heterocycle, -alkyl-OR⁸, -alkyl-NR⁸R⁹, C(O)R³, S(O)R³, C(S)R³, and S(O)₂R³;

R⁸ and R⁹ are independently selected at each occurrence from hydrogen, heteroalkyl, alkyl, arylalkyl, heteroarylalkyl, alkenyl, alkynyl, aryl, heteroaryl, and heterocycle.

A. Galactose-Based ASGPR-Binding Cellular Receptor Binding Moieties of Formula II

In certain embodiments, the compound of Formula II is selected from:

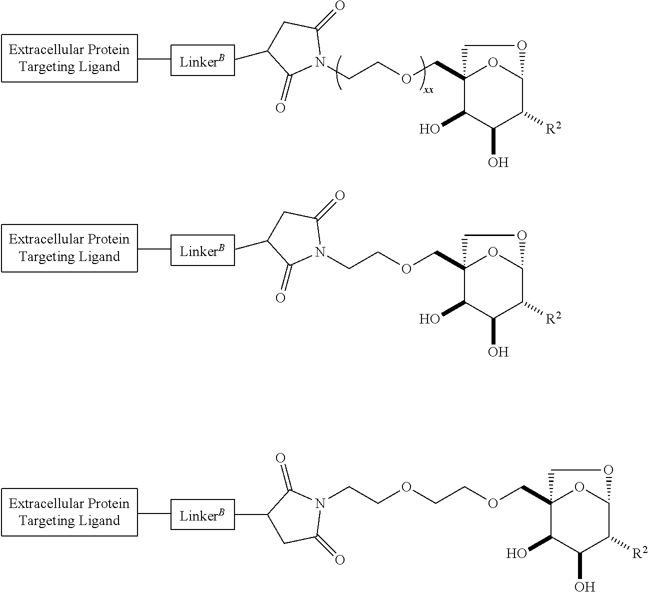

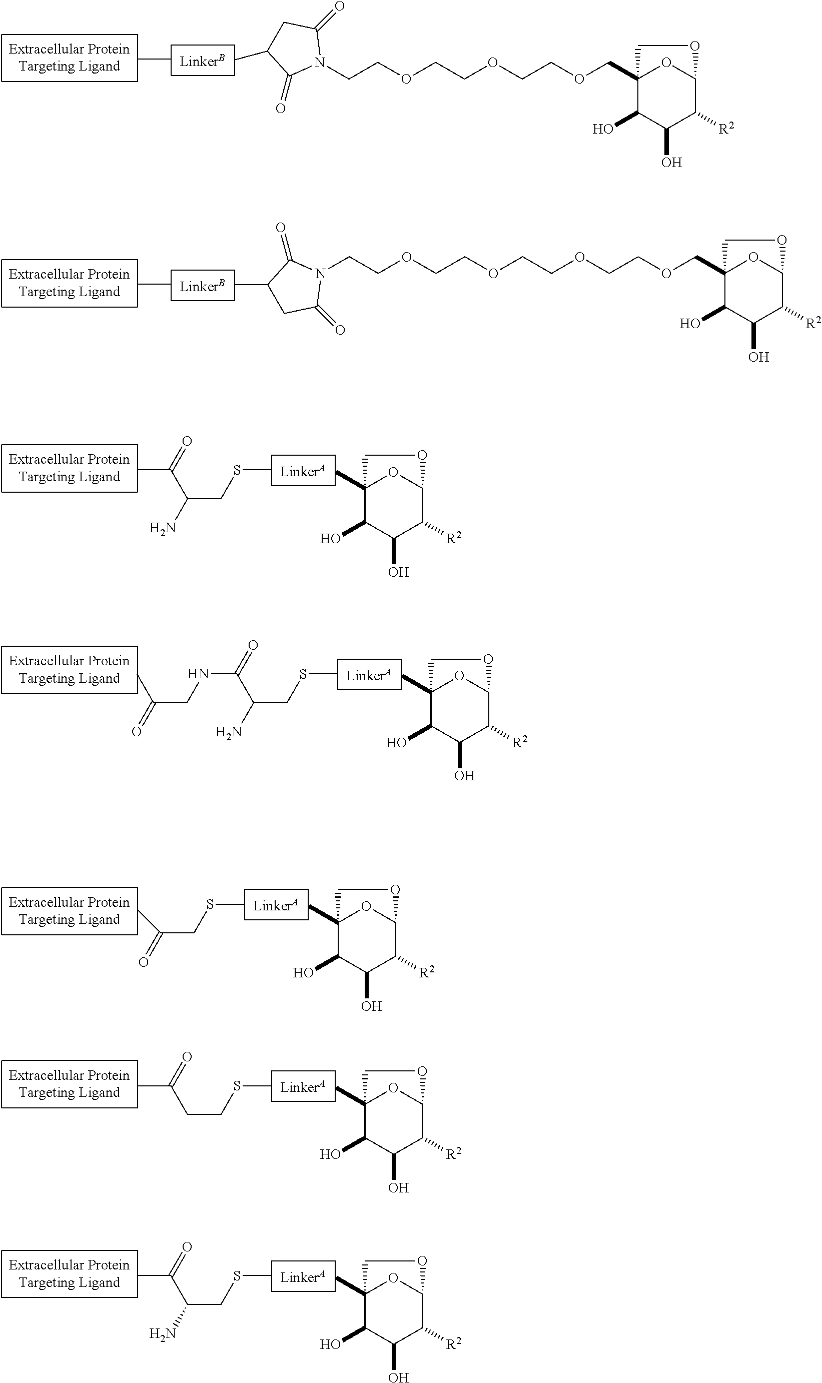

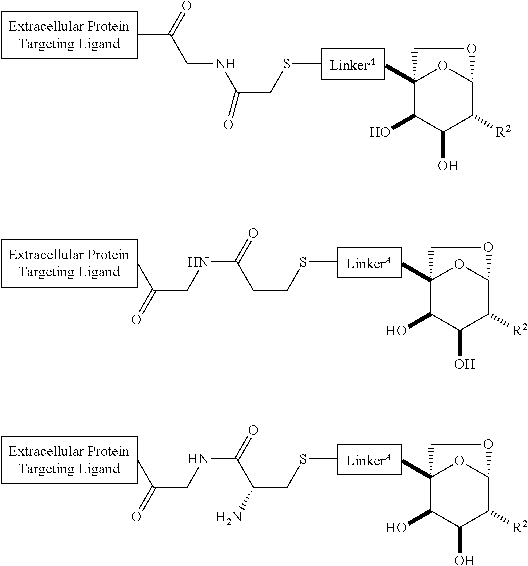

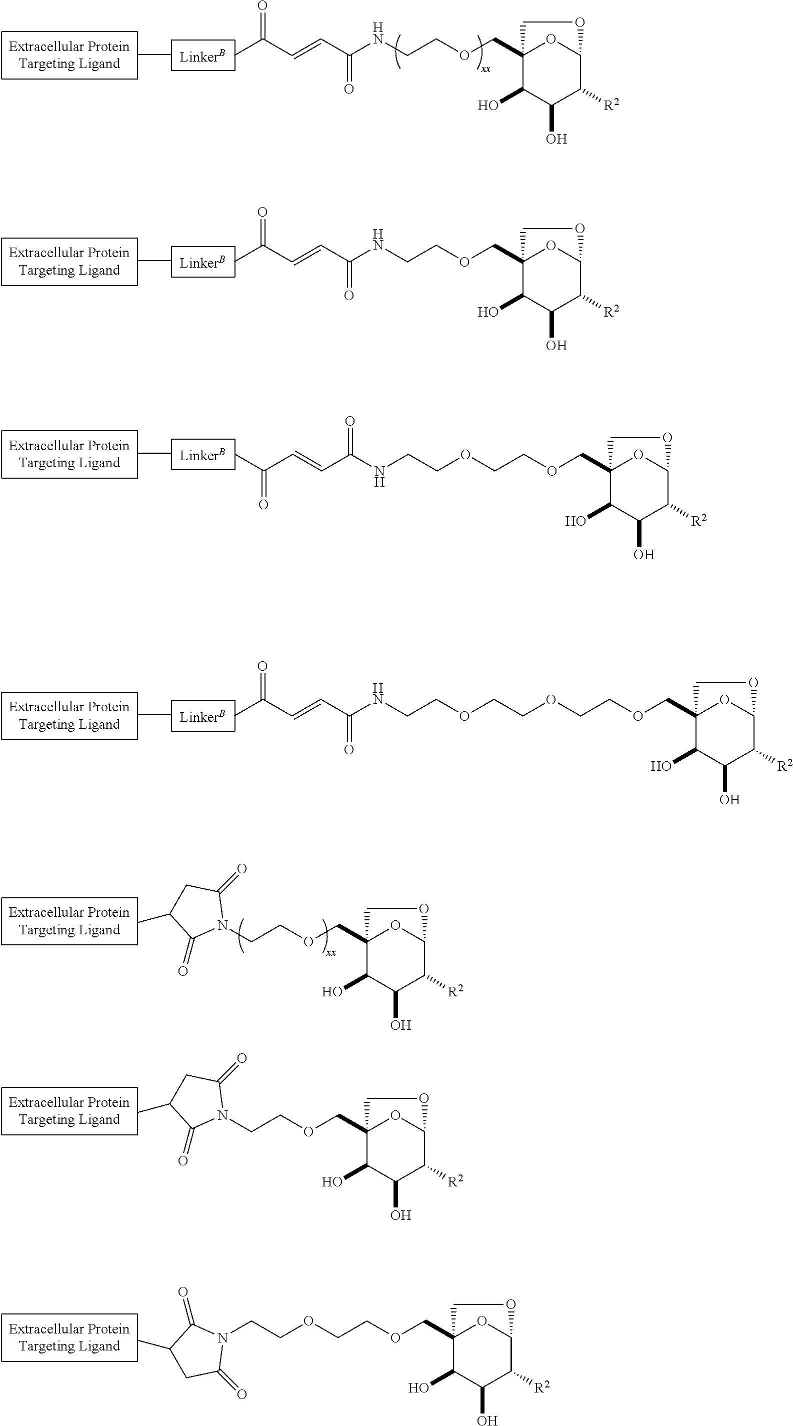

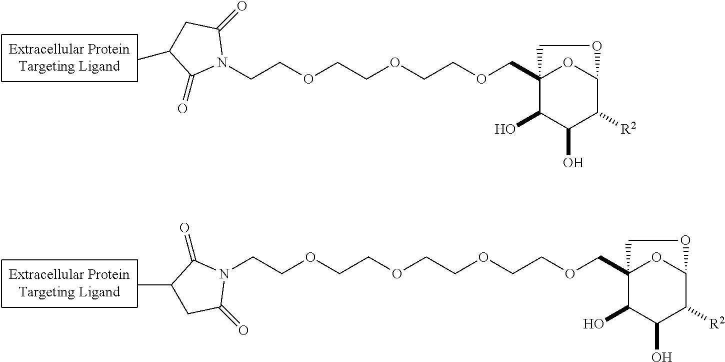

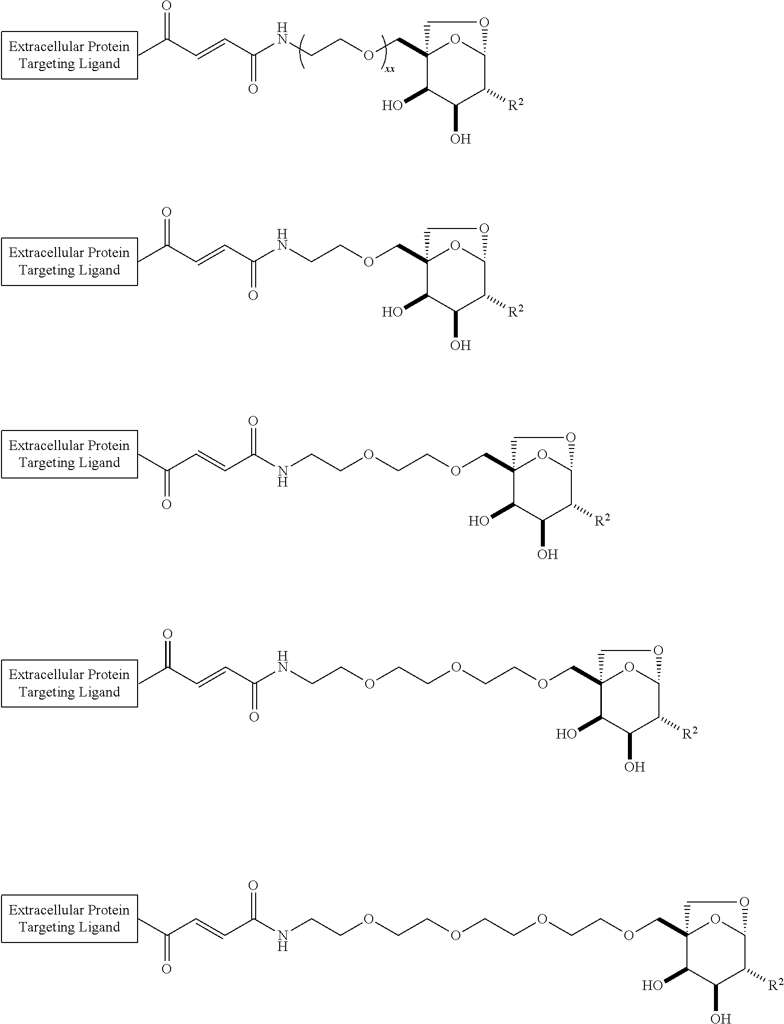

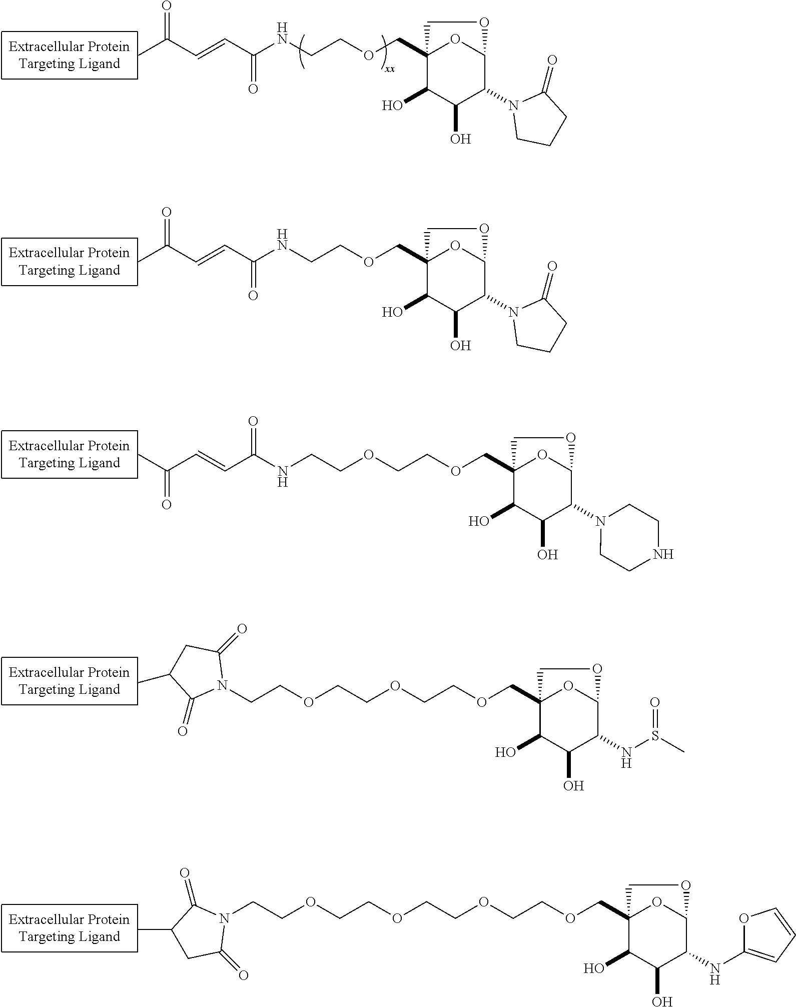

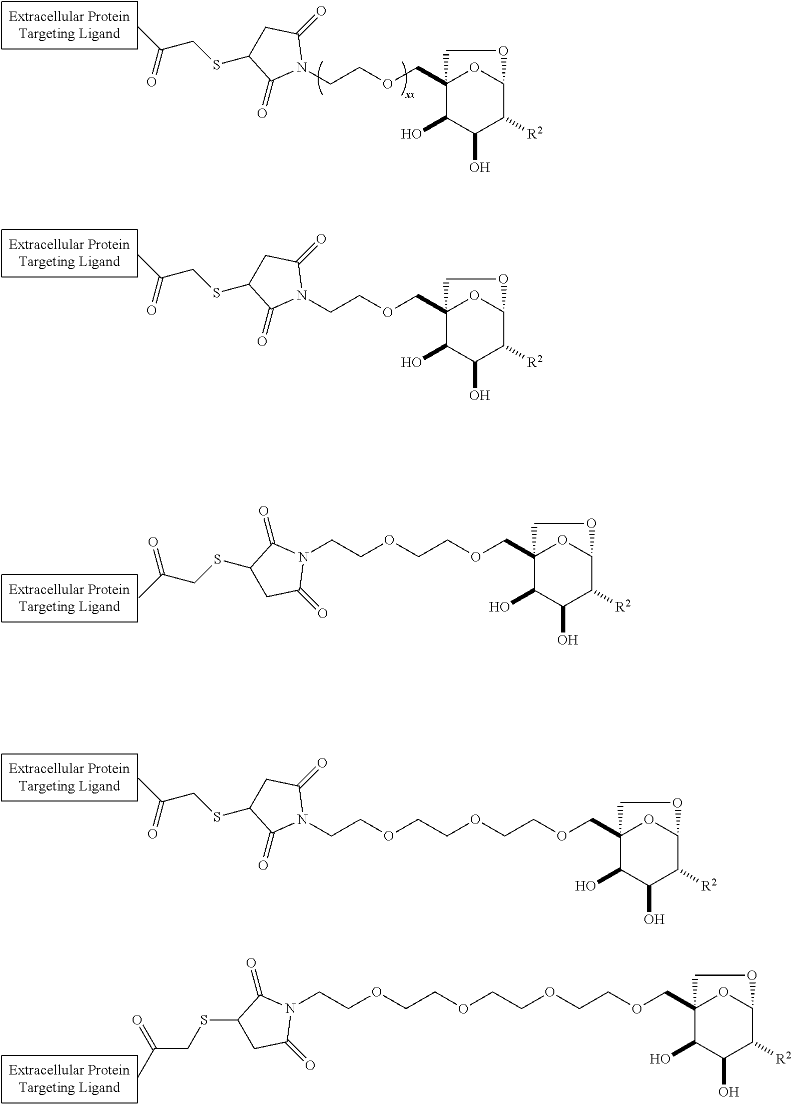

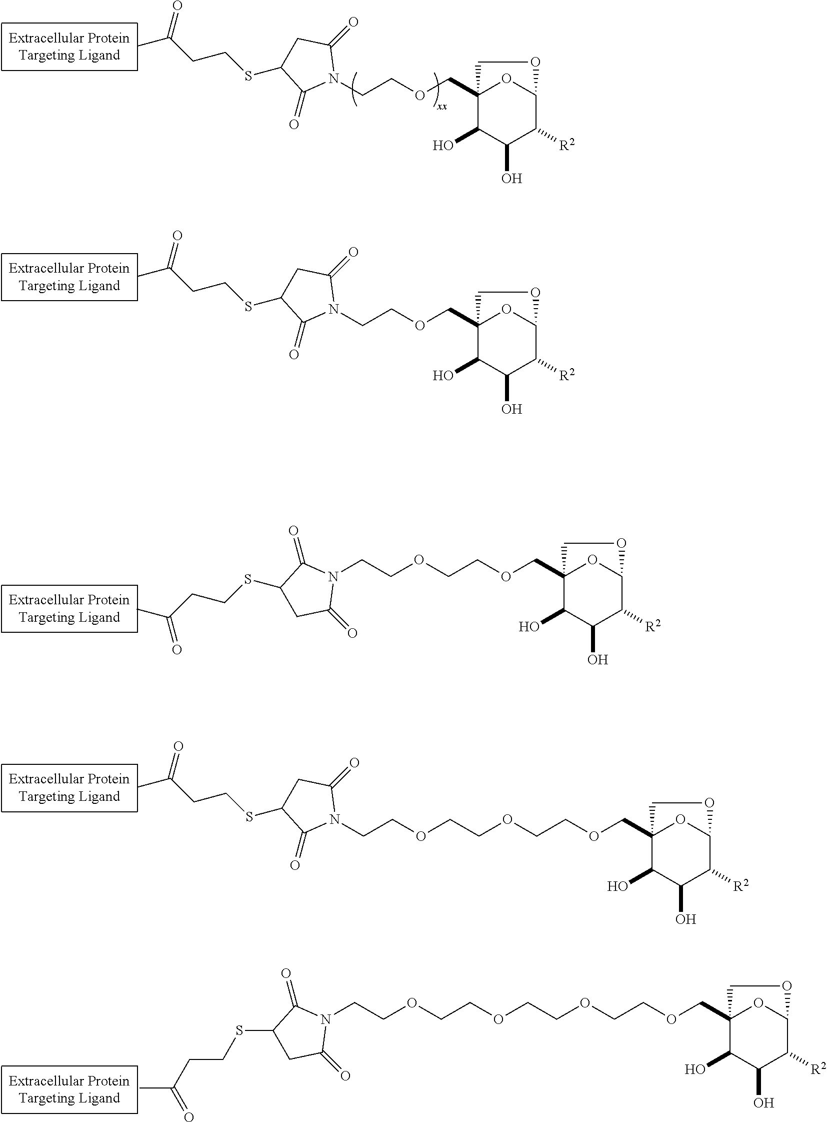

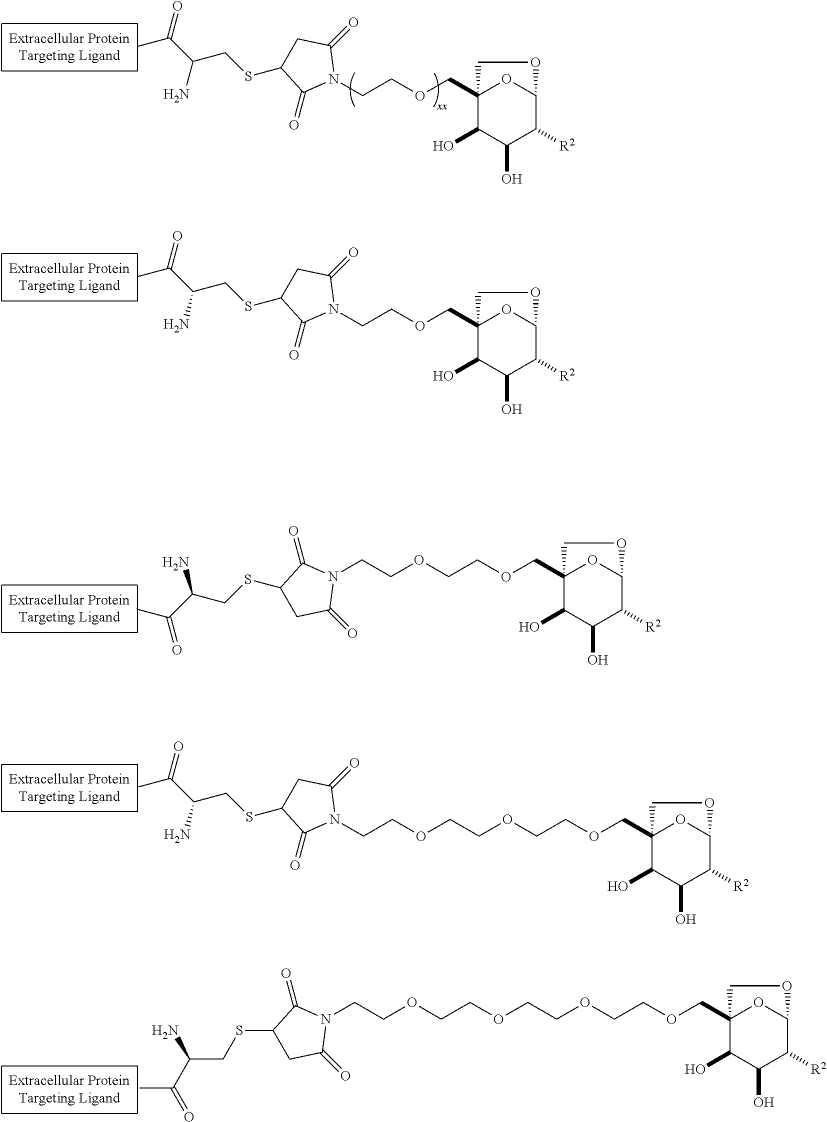

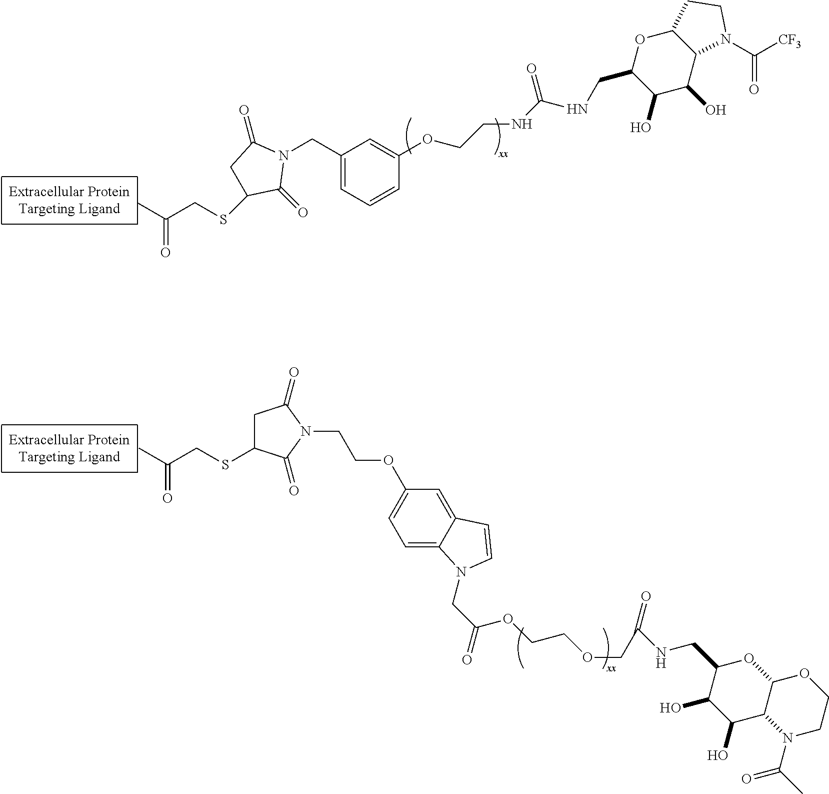

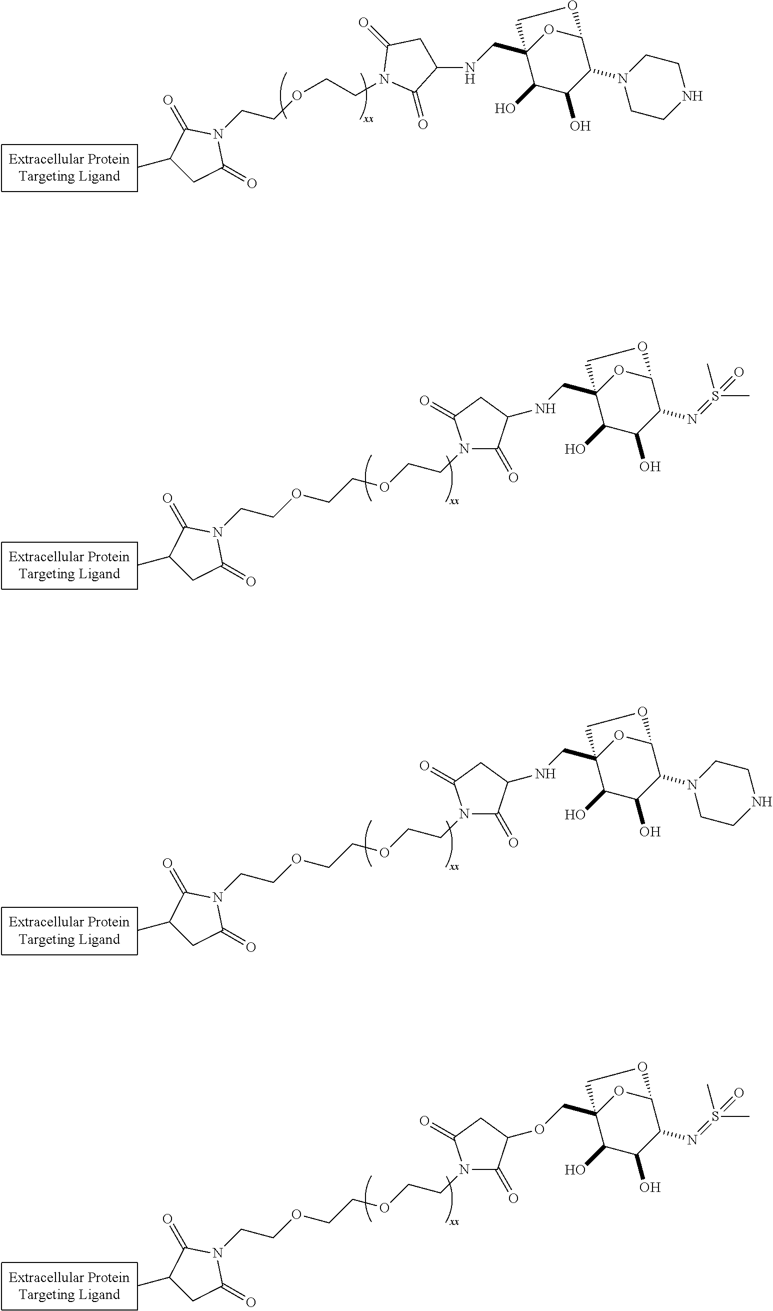

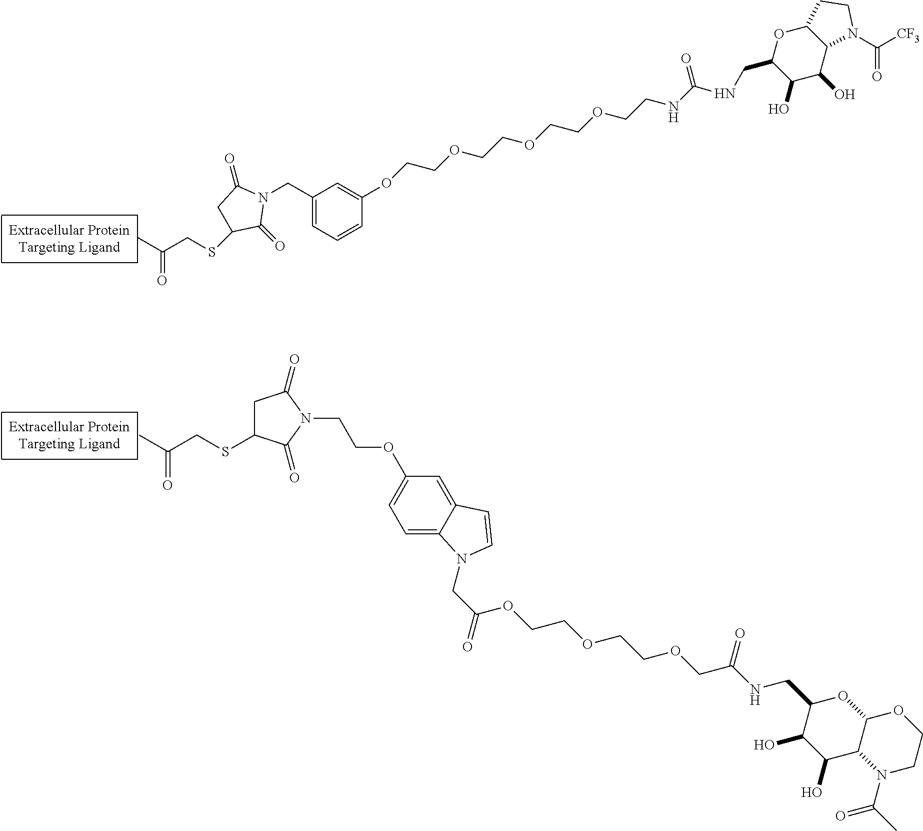

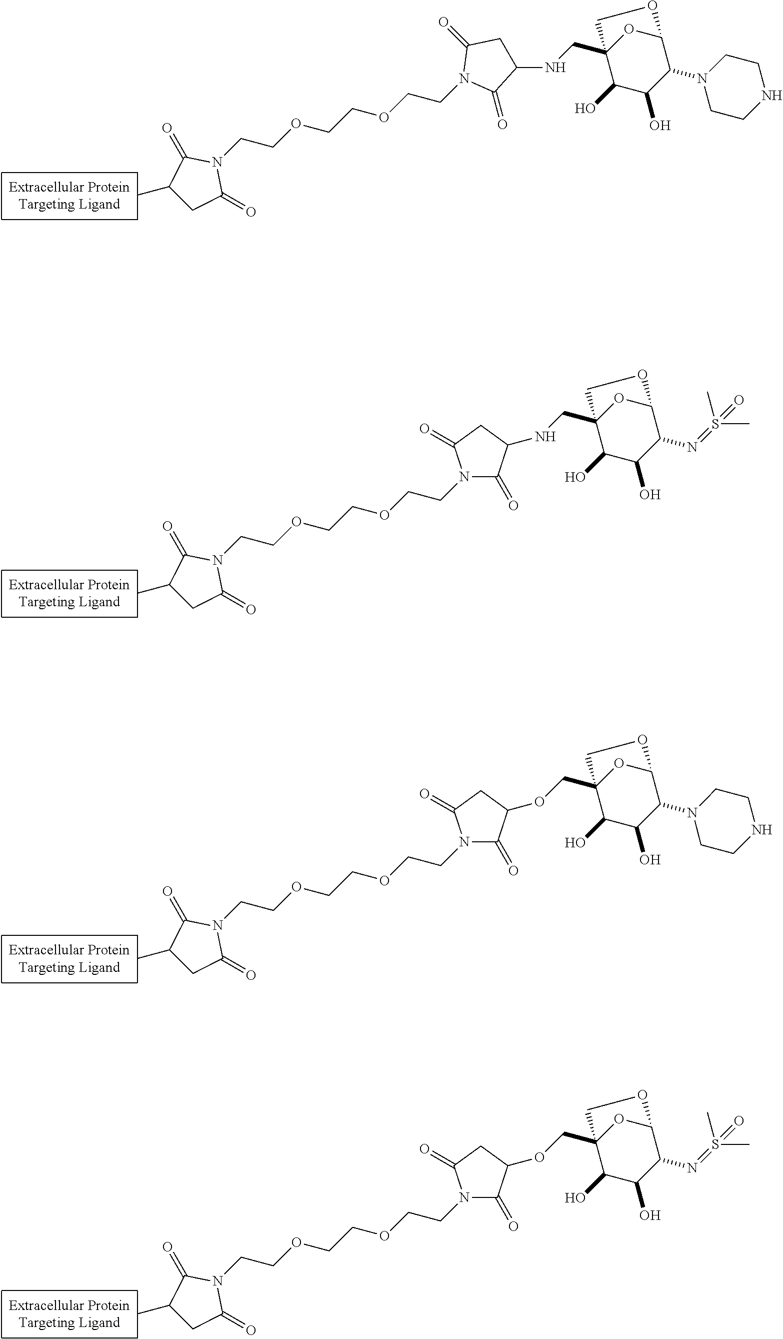

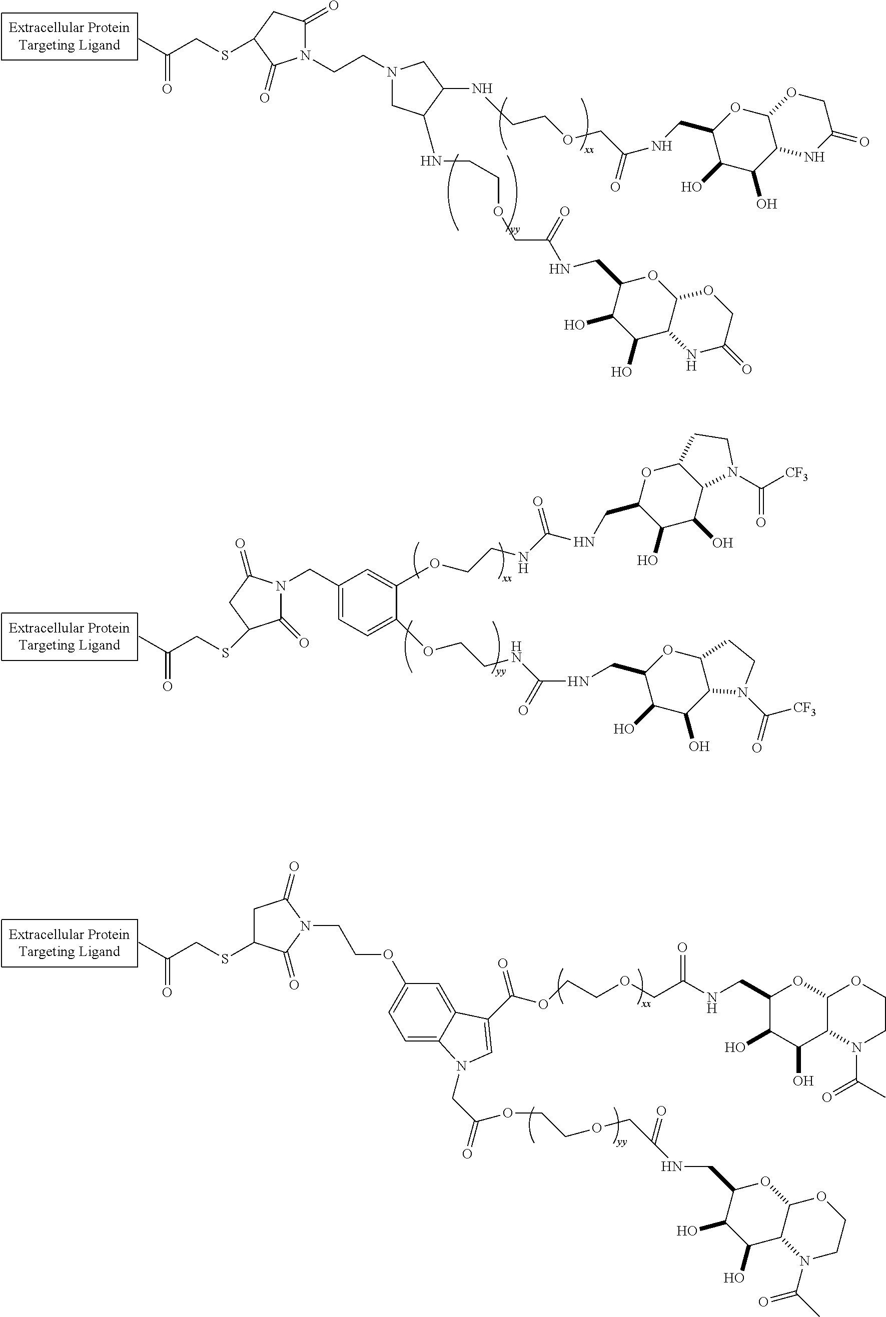

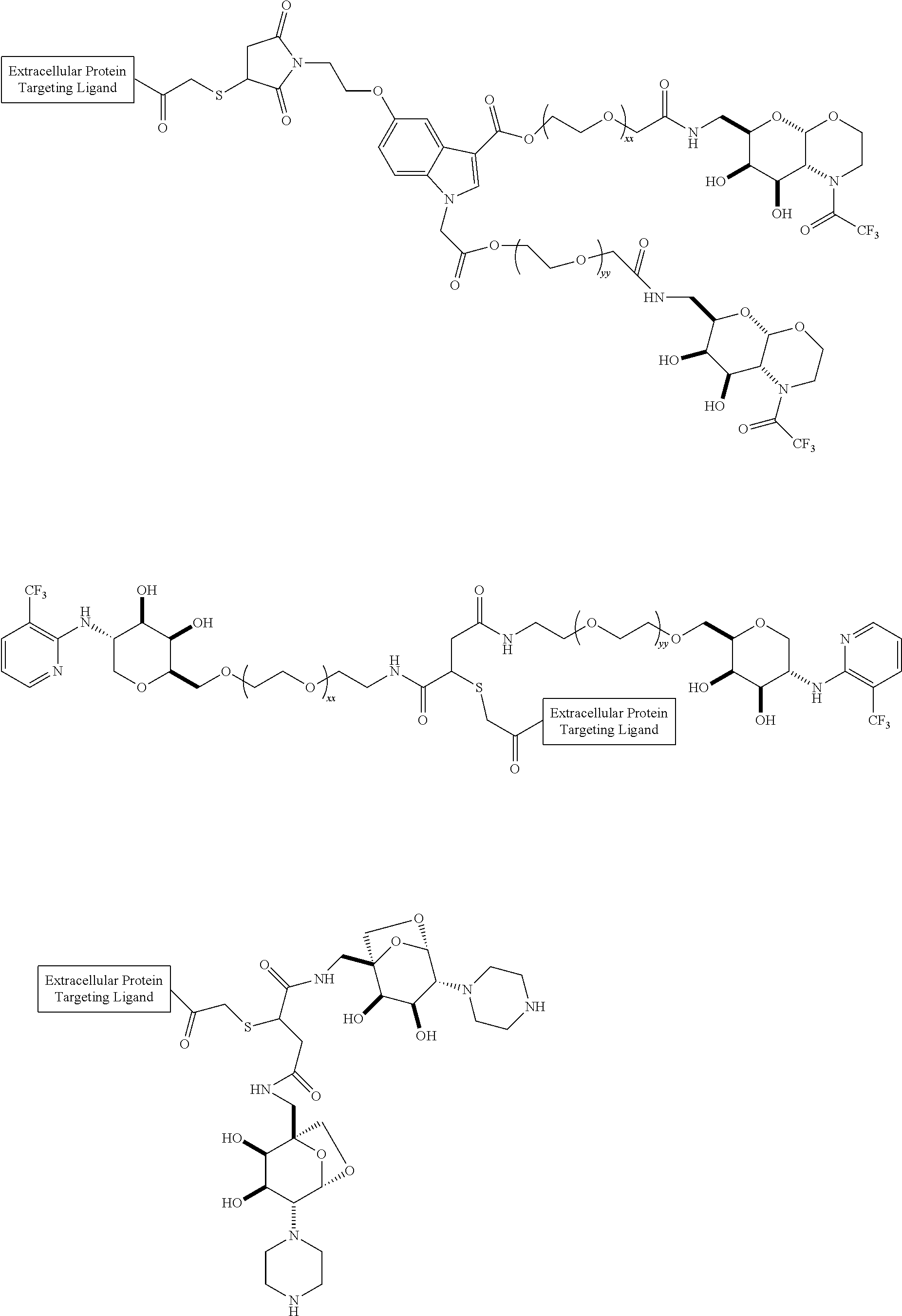

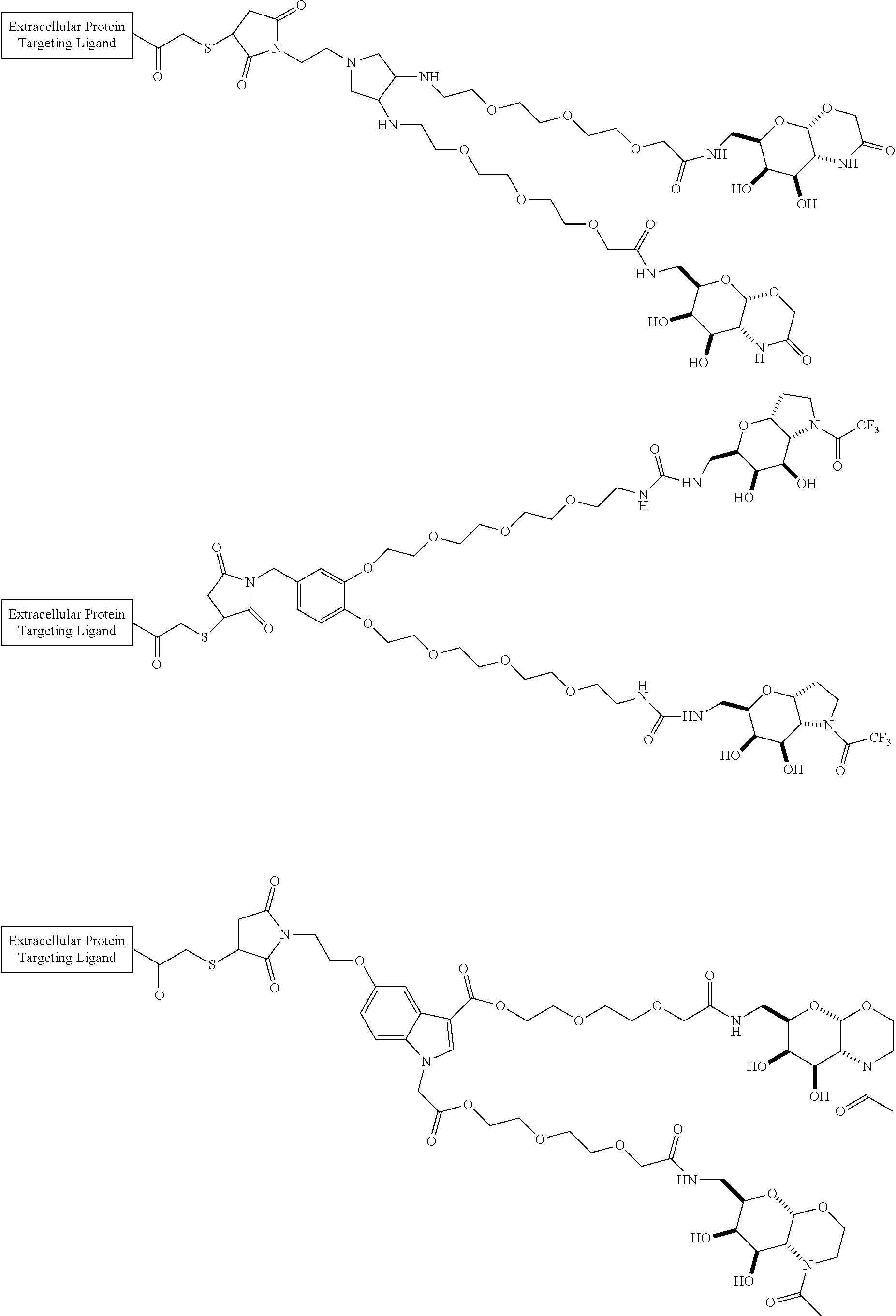

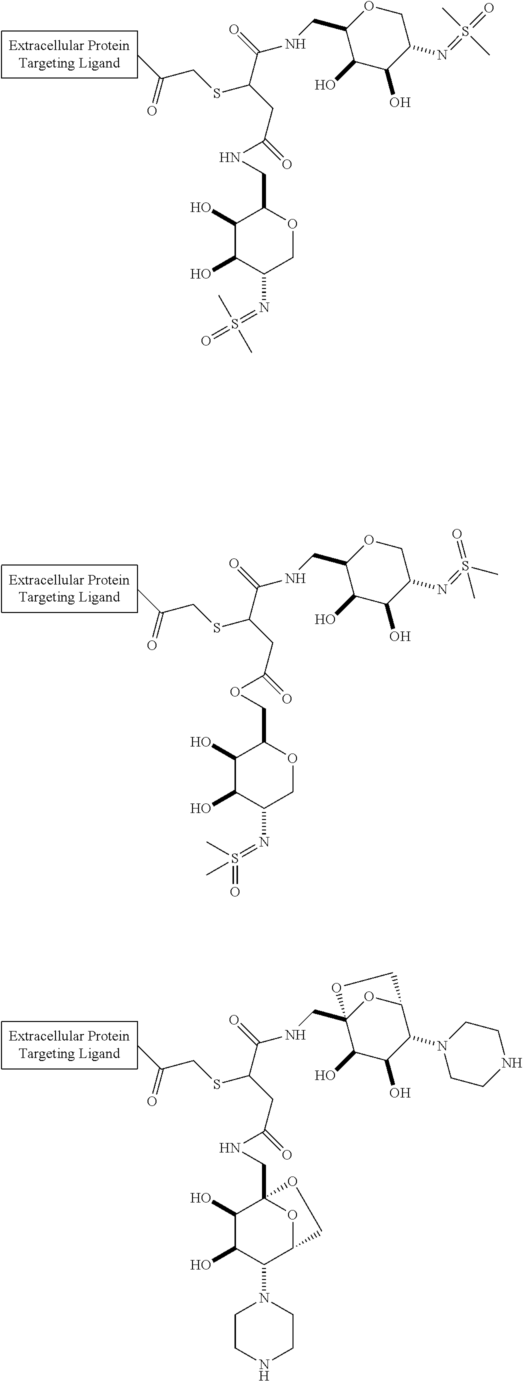

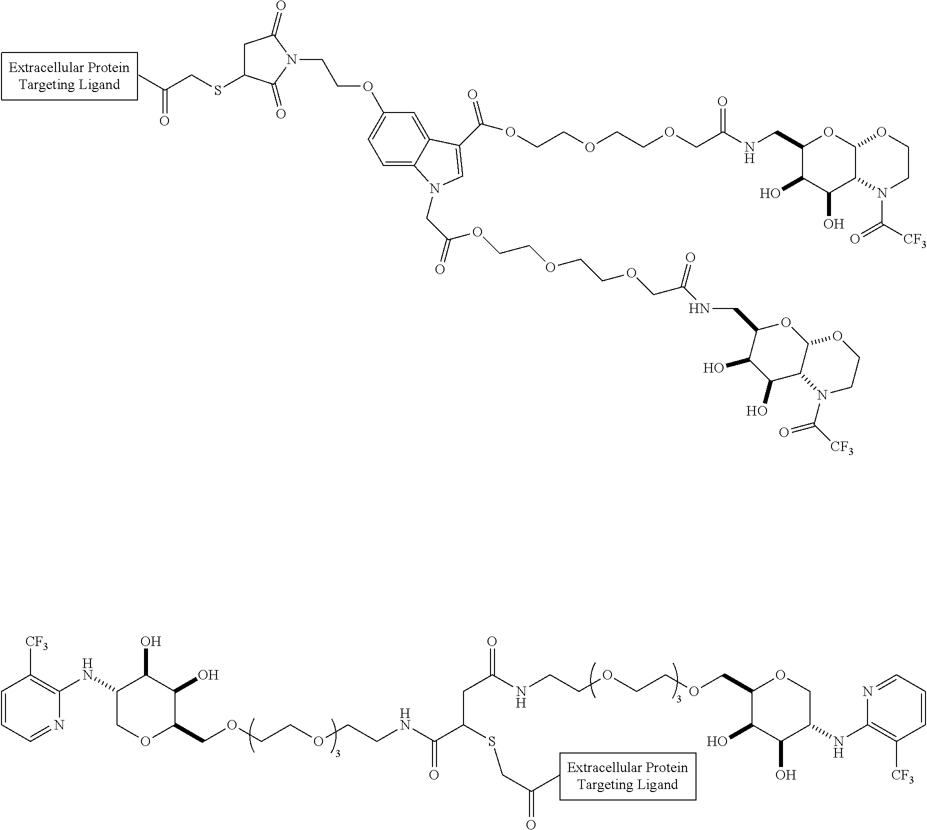

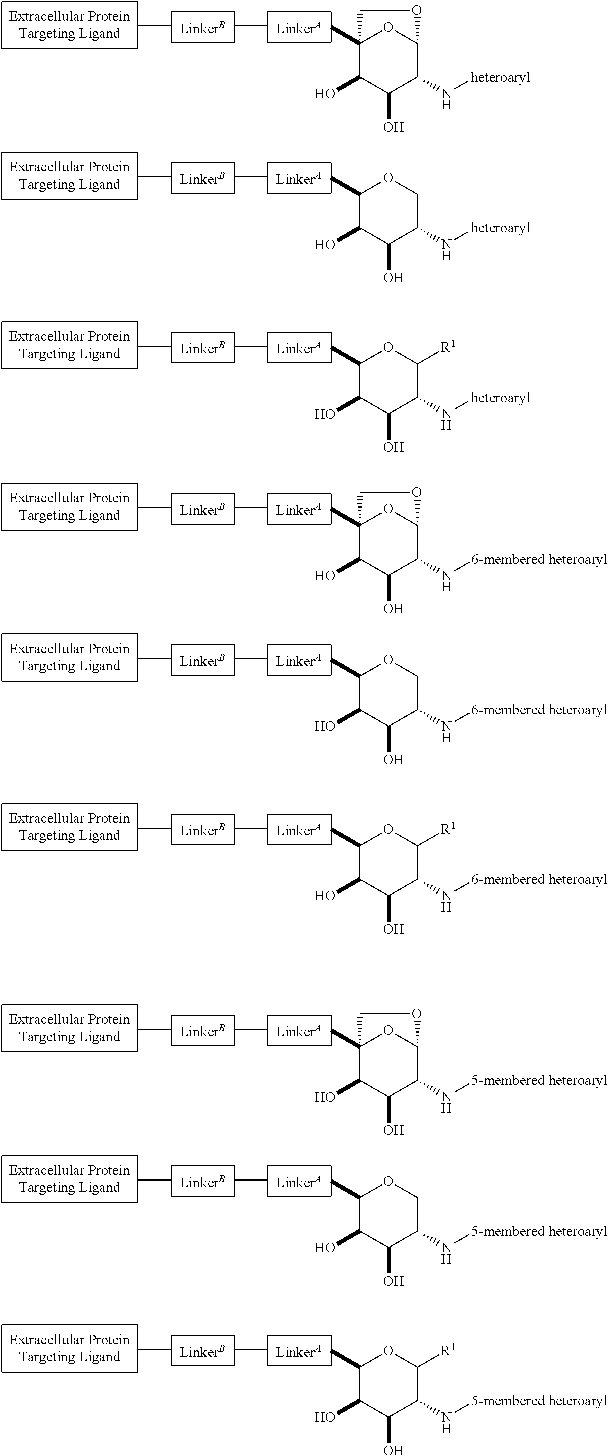

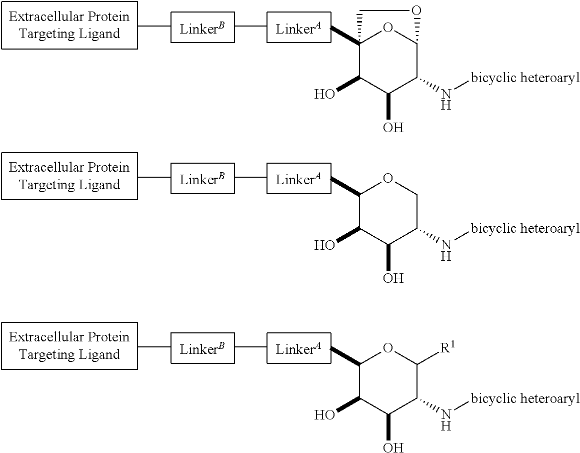

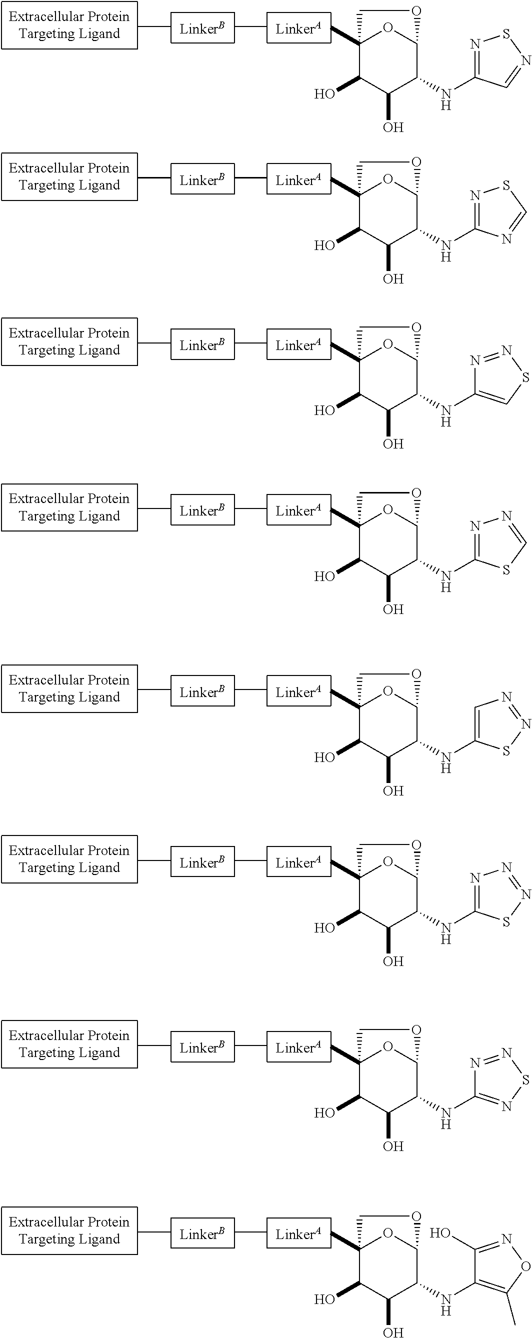

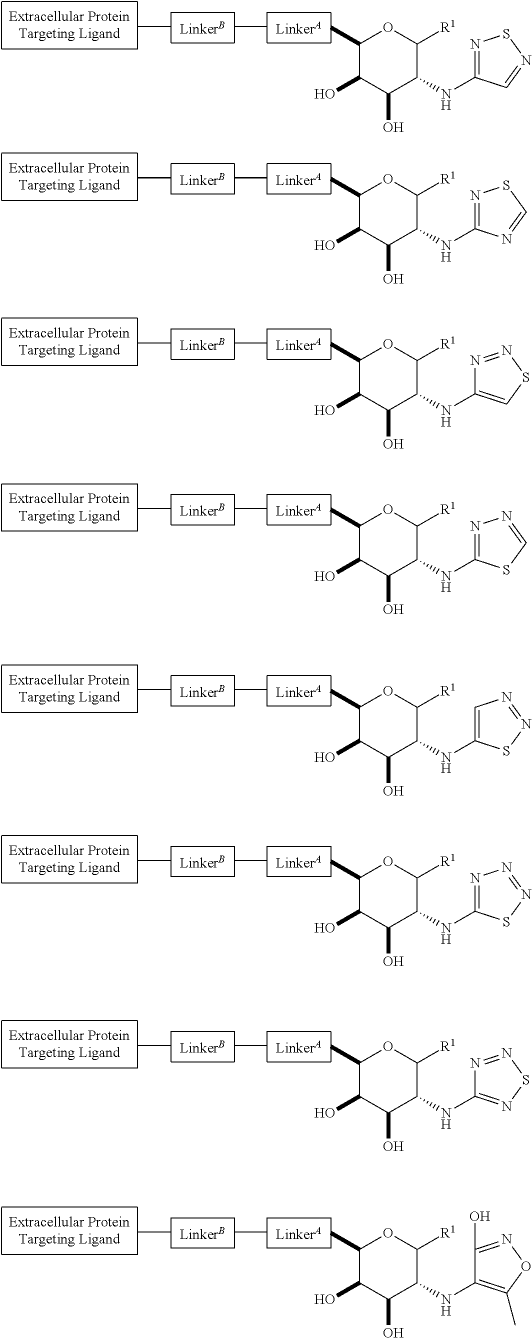

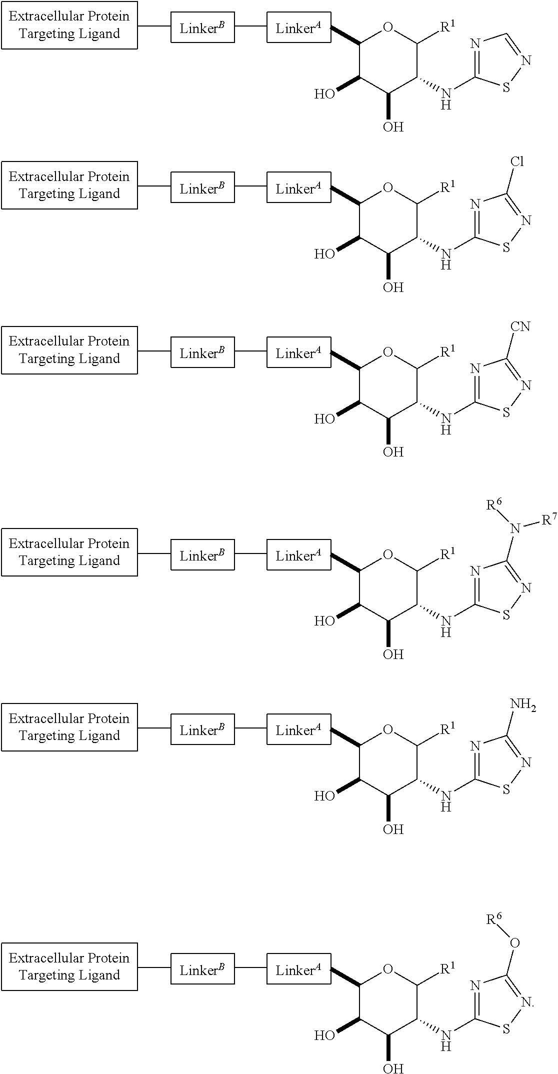

In one embodiment, the compound of Formula II has one of the following structures:

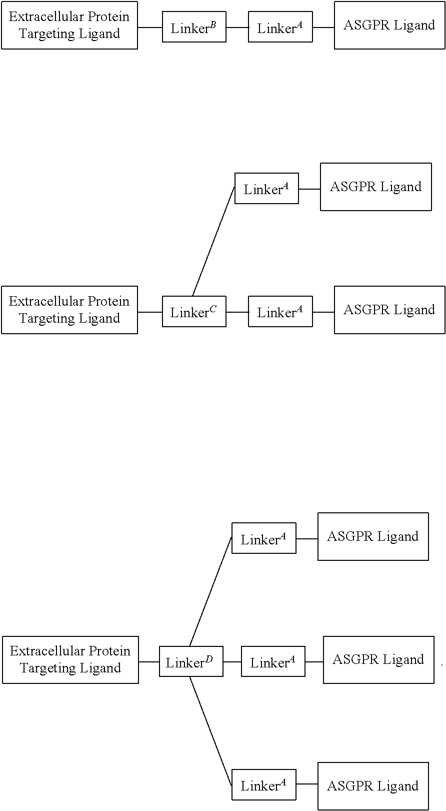

In various embodiments, the ASGPR ligand is linked at either the C¹ or C⁵ (R¹ or R⁵) position to form a degrading compound. In various embodiments, the ASGPR ligand is linked at C⁶ position to form a degrading compound. For example, when the ASGPR ligand is

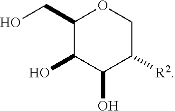

then non-limiting examples of ASGPR binding compounds of Formula II include:

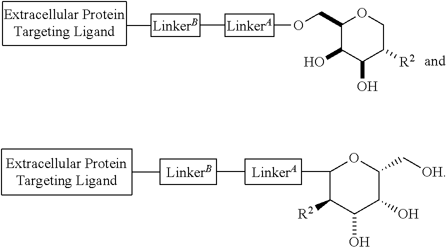

or the bi- or tri-substituted versions thereof or pharmaceutically acceptable salts thereof, where the bi- or tri-substitution refers to the number additional galactose derivatives attached to a linker moiety.

In any of the embodiments herein where an ASGPR ligand is drawn for use in a degrader the ASGPR ligand is typically linked through to the Extracellular Protein Targeting Ligand in the C⁵ position (e.g., which can refer to the adjacent C⁶ carbon hydroxyl or other functional moiety that can be used for linking purposes). When the linker and Extracellular Protein Targeting Ligand is connected through the C¹ position, then that carbon is appropriately functionalized for linking, for example with a hydroxyl, amino, allyl, alkyne or hydroxyl-allyl group.

In various embodiments, the ASGPR ligand is not linked in the C³ or C⁴ position, because these positions chelate with the calcium for ASGPR binding in the liver. In certain embodiments, an ASGPR ligand useful for incorporation into a compound of Formula II is selected from:

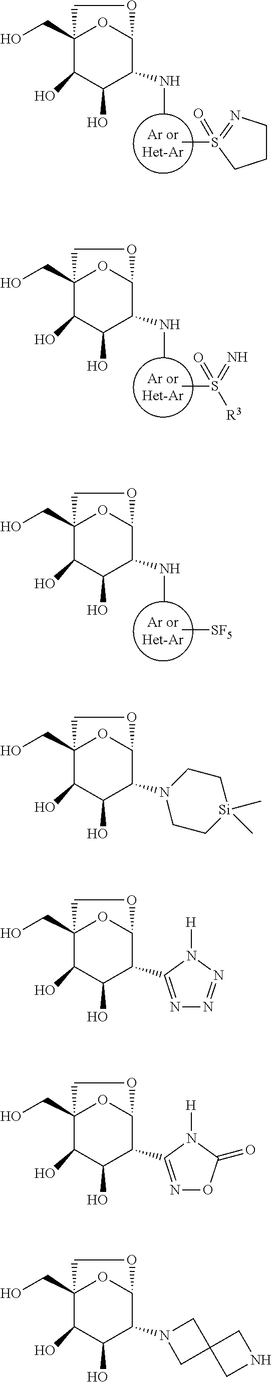

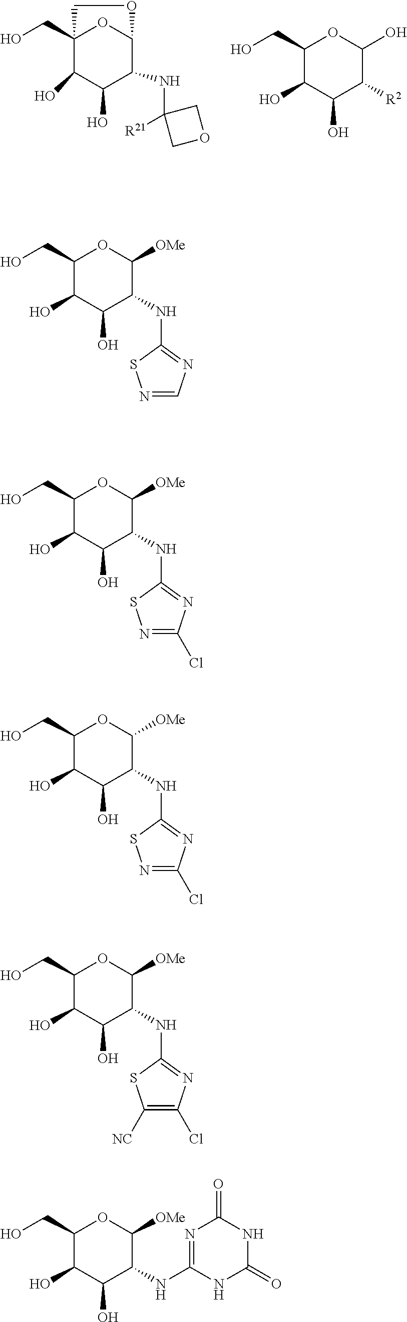

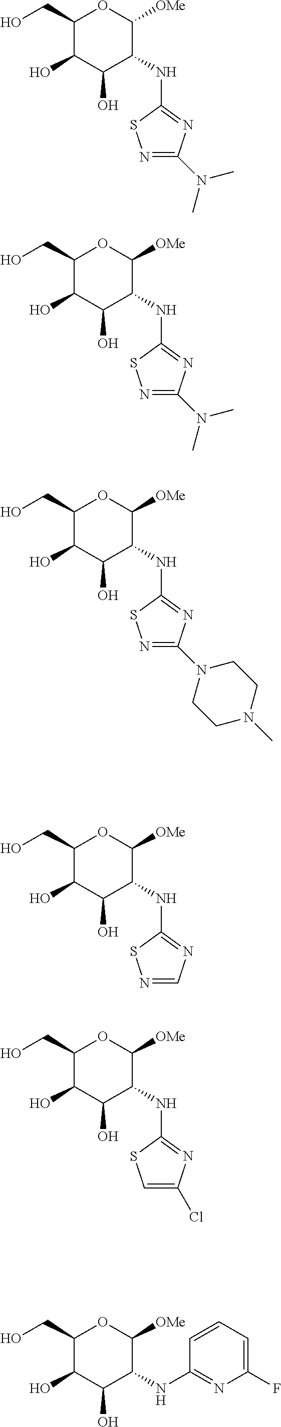

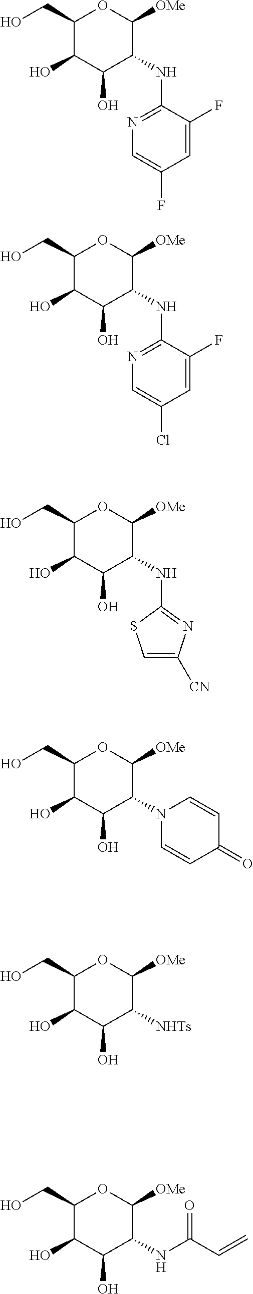

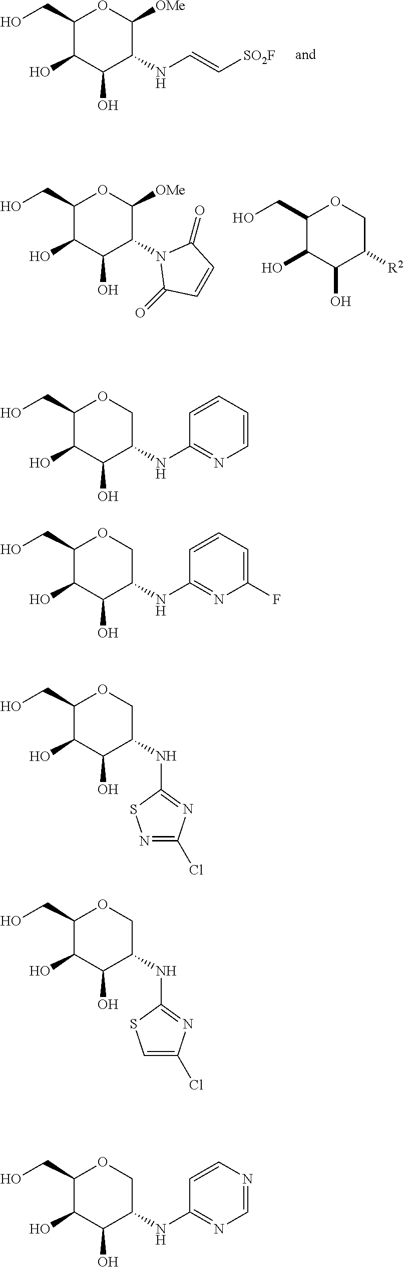

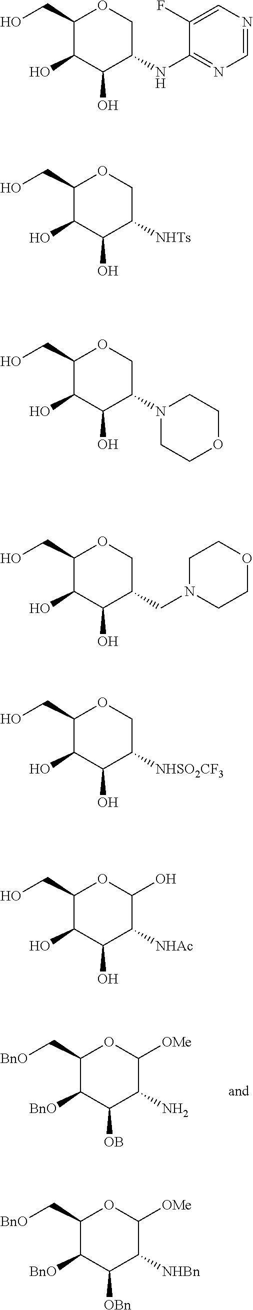

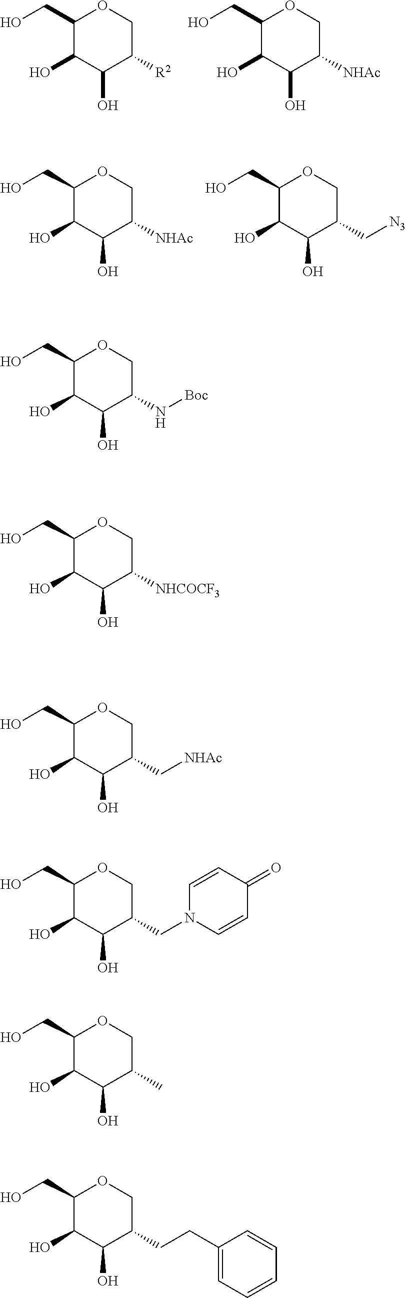

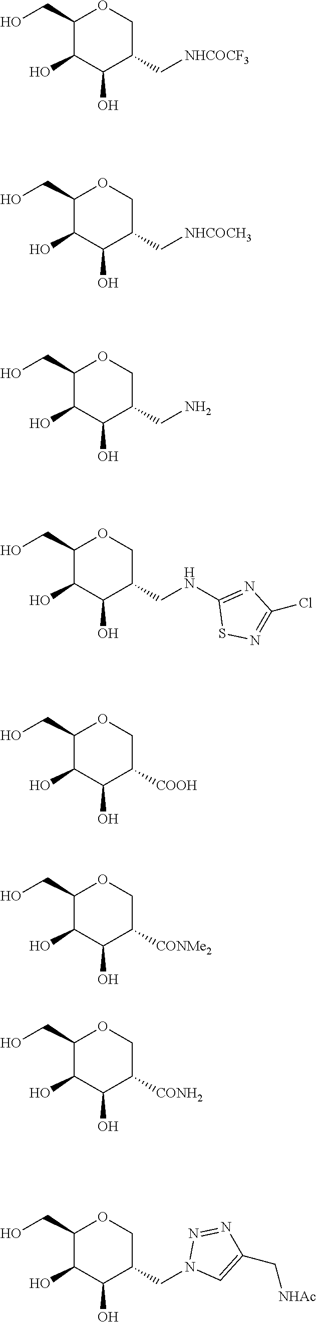

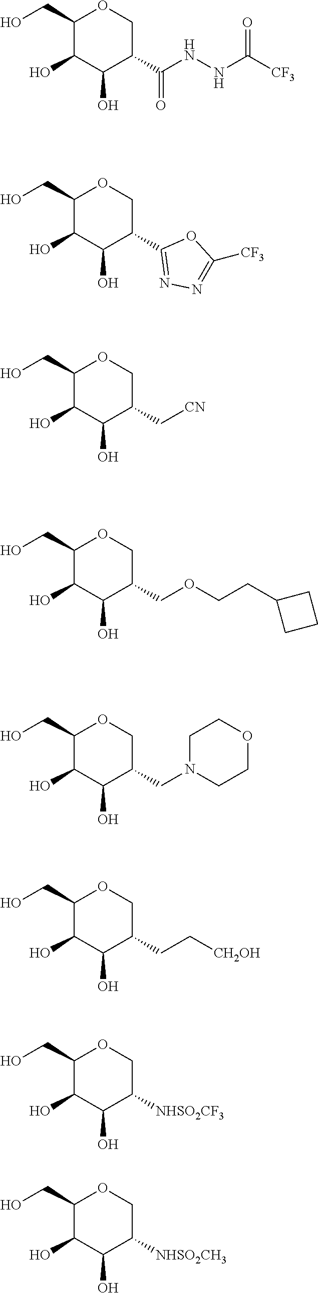

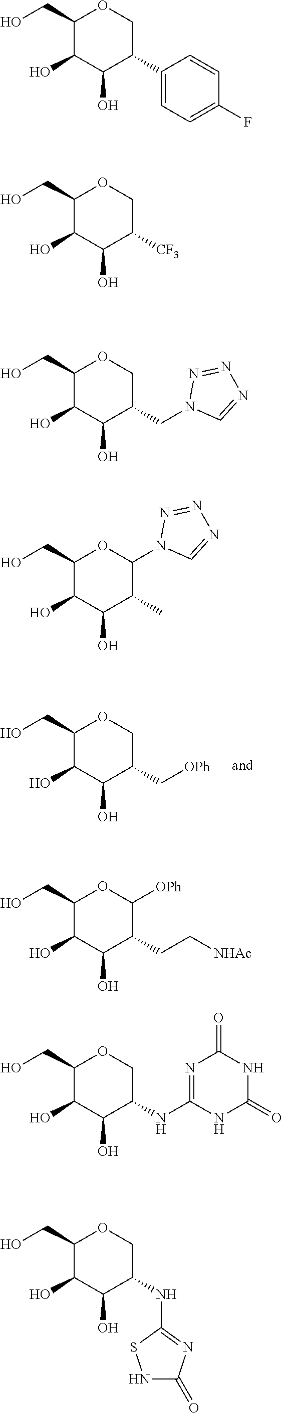

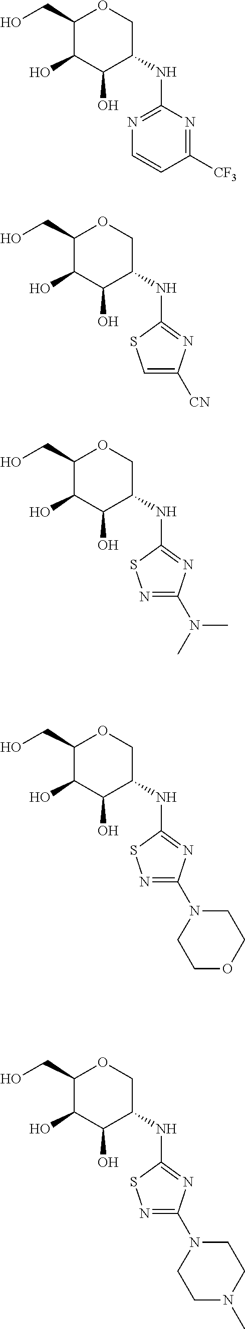 [SMILES: OC[C@H]1OC[C@H](Nc2nccc(C(F)(F)F)n2)[C@@H](O)[C@H]1O ; N#Cc1csc(N[C@H]2CO[C@H](CO)[C@H](O)[C@@H]2O)n1 ; CN(C)c1nsc(N[C@H]2CO[C@H](CO)[C@H](O)[C@@H]2O)n1 ; OC[C@H]1OC[C@H](Nc2nc(N3CCOCC3)ns2)[C@@H](O)[C@H]1O ; CN1CCN(c2nsc(N[C@H]3CO[C@H](CO)[C@H](O)[C@@H]3O)n2)CC1]

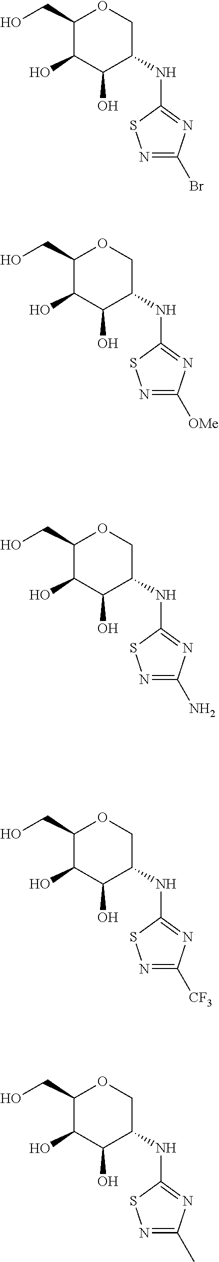 [SMILES: OC[C@H]1OC[C@H](Nc2nc(Br)ns2)[C@@H](O)[C@H]1O ; COc1nsc(N[C@H]2CO[C@H](CO)[C@H](O)[C@@H]2O)n1 ; Nc1nsc(N[C@H]2CO[C@H](CO)[C@H](O)[C@@H]2O)n1 ; OC[C@H]1OC[C@H](Nc2nc(C(F)(F)F)ns2)[C@@H](O)[C@H]1O ; Cc1nsc(N[C@H]2CO[C@H](CO)[C@H](O)[C@@H]2O)n1]

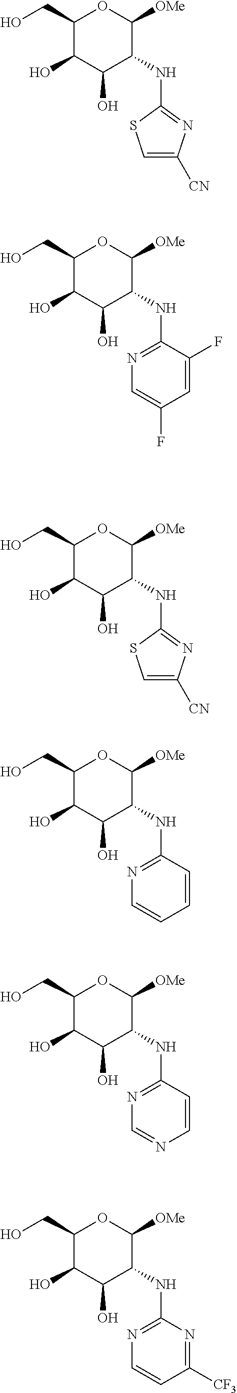 [SMILES: CO[C@@H]1O[C@H](CO)[C@H](O)[C@H](O)[C@H]1Nc1nc(C#N)cs1 ; CO[C@@H]1O[C@H](CO)[C@H](O)[C@H](O)[C@H]1Nc1ncc(F)cc1F ; CO[C@@H]1O[C@H](CO)[C@H](O)[C@H](O)[C@H]1Nc1nc(C#N)cs1 ; CO[C@@H]1O[C@H](CO)[C@H](O)[C@H](O)[C@H]1Nc1ccccn1 ; CO[C@@H]1O[C@H](CO)[C@H](O)[C@H](O)[C@H]1Nc1ccncn1 ; CO[C@@H]1O[C@H](CO)[C@H](O)[C@H](O)[C@H]1Nc1nccc(C(F)(F)F)n1]

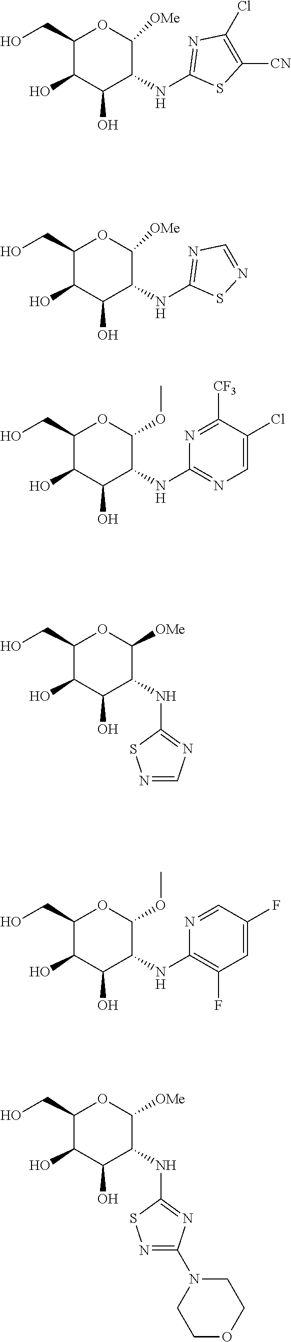 [SMILES: CO[C@H]1O[C@H](CO)[C@H](O)[C@H](O)[C@H]1Nc1nc(Cl)c(C#N)s1 ; CO[C@H]1O[C@H](CO)[C@H](O)[C@H](O)[C@H]1Nc1ncns1 ; CO[C@H]1O[C@H](CO)[C@H](O)[C@H](O)[C@H]1Nc1ncc(Cl)c(C(F)(F)F)n1 ; CO[C@@H]1O[C@H](CO)[C@H](O)[C@H](O)[C@H]1Nc1ncns1 ; CO[C@H]1O[C@H](CO)[C@H](O)[C@H](O)[C@H]1Nc1ncc(F)cc1F ; CO[C@H]1O[C@H](CO)[C@H](O)[C@H](O)[C@H]1Nc1nc(N2CCOCC2)ns1]

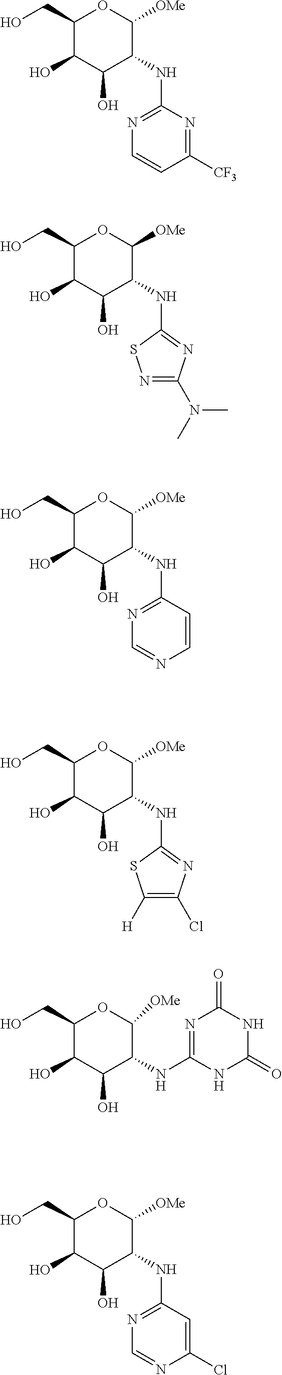 [SMILES: CO[C@H]1O[C@H](CO)[C@H](O)[C@H](O)[C@H]1Nc1nccc(C(F)(F)F)n1 ; CO[C@@H]1O[C@H](CO)[C@H](O)[C@H](O)[C@H]1Nc1nc(N(C)C)ns1 ; CO[C@H]1O[C@H](CO)[C@H](O)[C@H](O)[C@H]1Nc1ccncn1 ; CO[C@H]1O[C@H](CO)[C@H](O)[C@H](O)[C@H]1Nc1nc(Cl)cs1 ; CO[C@H]1O[C@H](CO)[C@H](O)[C@H](O)[C@H]1Nc1nc(=O)[nH]c(=O)[nH]1 ; CO[C@H]1O[C@H](CO)[C@H](O)[C@H](O)[C@H]1Nc1cc(Cl)ncn1]

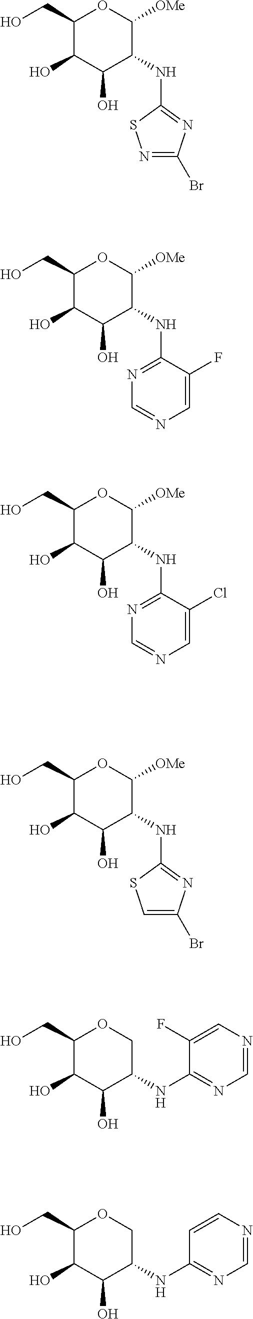 [SMILES: CO[C@H]1O[C@H](CO)[C@H](O)[C@H](O)[C@H]1Nc1nc(Br)ns1 ; CO[C@H]1O[C@H](CO)[C@H](O)[C@H](O)[C@H]1Nc1ncncc1F ; CO[C@H]1O[C@H](CO)[C@H](O)[C@H](O)[C@H]1Nc1ncncc1Cl ; CO[C@H]1O[C@H](CO)[C@H](O)[C@H](O)[C@H]1Nc1nc(Br)cs1 ; OC[C@H]1OC[C@H](Nc2ncncc2F)[C@@H](O)[C@H]1O ; OC[C@H]1OC[C@H](Nc2ccncn2)[C@@H](O)[C@H]1O]

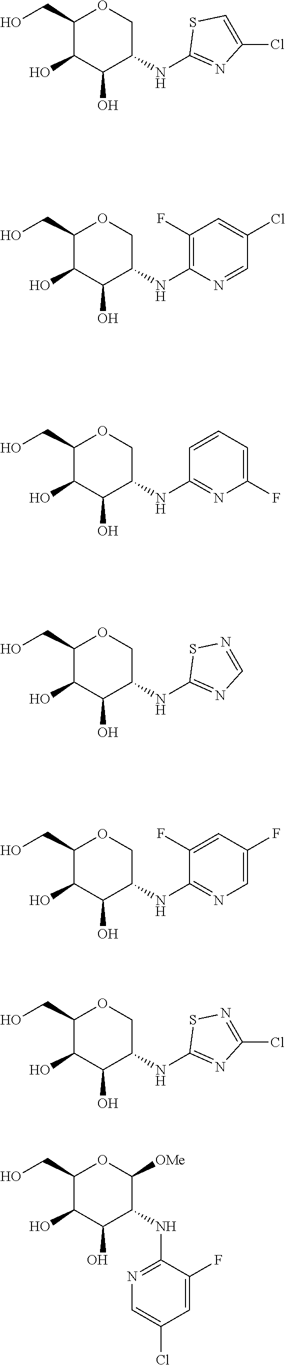

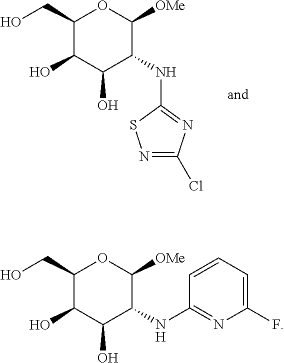 [SMILES: CO[C@@H]1O[C@H](CO)[C@H](O)[C@H](O)[C@H]1Nc1nc(Cl)ns1 ; CO[C@@H]1O[C@H](CO)[C@H](O)[C@H](O)[C@H]1Nc1cccc(F)n1]

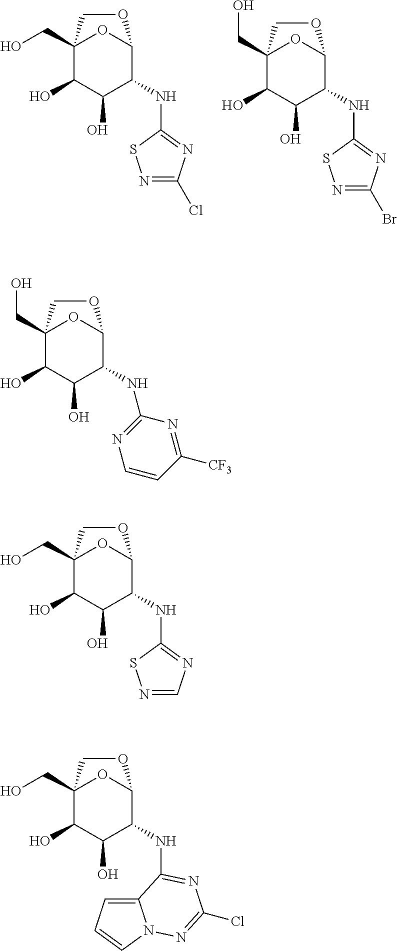 [SMILES: OC[C@@]12CO[C@@H](O1)[C@H](Nc1nc(Cl)ns1)[C@@H](O)[C@H]2O ; OC[C@@]12CO[C@@H](O1)[C@H](Nc1nc(Br)ns1)[C@@H](O)[C@H]2O ; OC[C@@]12CO[C@@H](O1)[C@H](Nc1nccc(C(F)(F)F)n1)[C@@H](O)[C@H]2O ; OC[C@@]12CO[C@@H](O1)[C@H](Nc1ncns1)[C@@H](O)[C@H]2O ; OC[C@@]12CO[C@@H](O1)[C@H](Nc1nc(Cl)nn3cccc13)[C@@H](O)[C@H]2O]

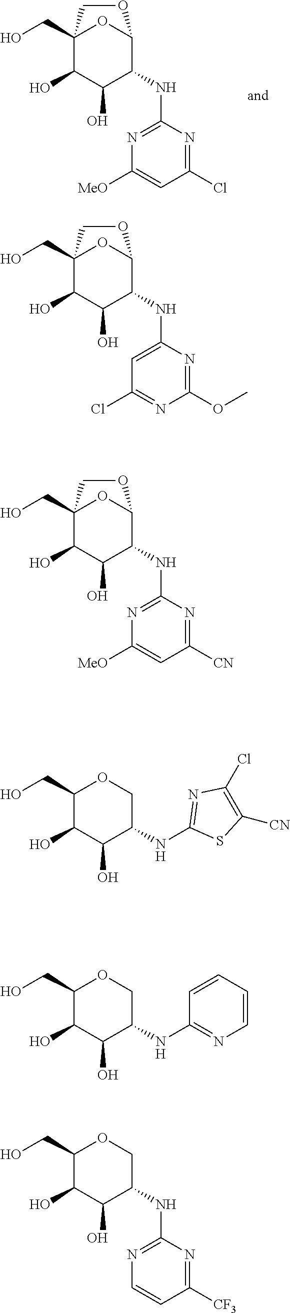

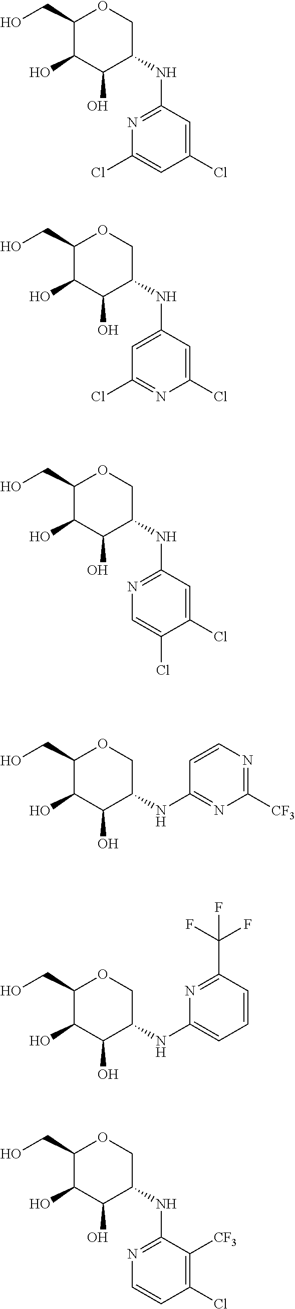 [SMILES: OC[C@H]1OC[C@H](Nc2cc(Cl)cc(Cl)n2)[C@@H](O)[C@H]1O ; OC[C@H]1OC[C@H](Nc2cc(Cl)nc(Cl)c2)[C@@H](O)[C@H]1O ; OC[C@H]1OC[C@H](Nc2cc(Cl)c(Cl)cn2)[C@@H](O)[C@H]1O ; OC[C@H]1OC[C@H](Nc2ccnc(C(F)(F)F)n2)[C@@H](O)[C@H]1O ; OC[C@H]1OC[C@H](Nc2cccc(C(F)(F)F)n2)[C@@H](O)[C@H]1O ; OC[C@H]1OC[C@H](Nc2nccc(Cl)c2C(F)(F)F)[C@@H](O)[C@H]1O]

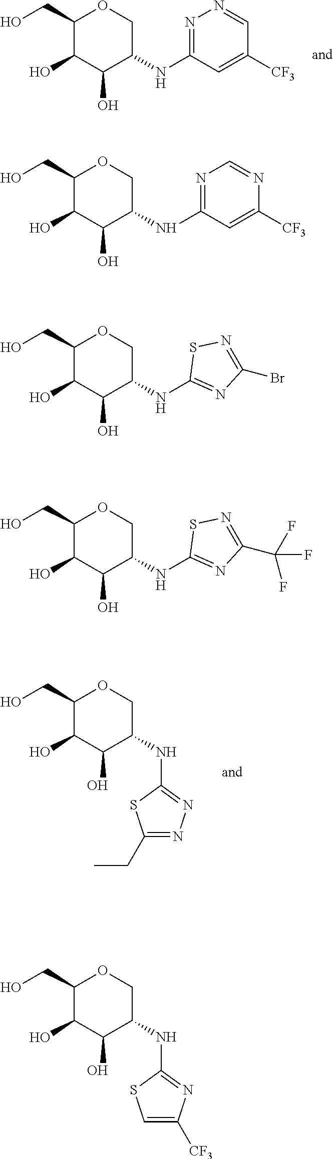 [SMILES: OC[C@H]1OC[C@H](Nc2cc(C(F)(F)F)cnn2)[C@@H](O)[C@H]1O ; OC[C@H]1OC[C@H](Nc2cc(C(F)(F)F)ncn2)[C@@H](O)[C@H]1O ; OC[C@H]1OC[C@H](Nc2nc(Br)ns2)[C@@H](O)[C@H]1O ; OC[C@H]1OC[C@H](Nc2nc(C(F)(F)F)ns2)[C@@H](O)[C@H]1O ; CCc1nnc(N[C@H]2CO[C@H](CO)[C@H](O)[C@@H]2O)s1 ; OC[C@H]1OC[C@H](Nc2nc(C(F)(F)F)cs2)[C@@H](O)[C@H]1O]

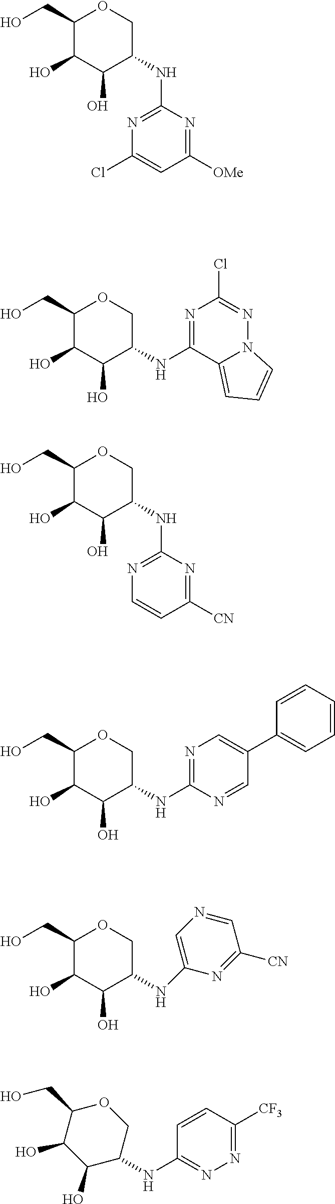 [SMILES: COc1cc(Cl)nc(N[C@H]2CO[C@H](CO)[C@H](O)[C@@H]2O)n1 ; OC[C@H]1OC[C@H](Nc2nc(Cl)nn3cccc23)[C@@H](O)[C@H]1O ; N#Cc1ccnc(N[C@H]2CO[C@H](CO)[C@H](O)[C@@H]2O)n1 ; OC[C@H]1OC[C@H](Nc2ncc(-c3ccccc3)cn2)[C@@H](O)[C@H]1O ; N#Cc1cncc(N[C@H]2CO[C@H](CO)[C@H](O)[C@@H]2O)n1 ; OC[C@H]1OC[C@H](Nc2ccc(C(F)(F)F)nn2)[C@@H](O)[C@H]1O]

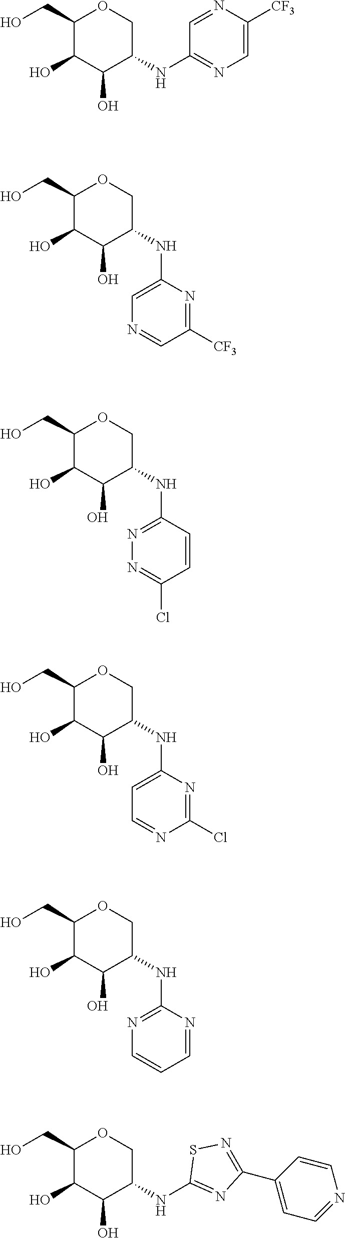 [SMILES: OC[C@H]1OC[C@H](Nc2cnc(C(F)(F)F)cn2)[C@@H](O)[C@H]1O ; OC[C@H]1OC[C@H](Nc2cncc(C(F)(F)F)n2)[C@@H](O)[C@H]1O ; OC[C@H]1OC[C@H](Nc2ccc(Cl)nn2)[C@@H](O)[C@H]1O ; OC[C@H]1OC[C@H](Nc2ccnc(Cl)n2)[C@@H](O)[C@H]1O ; OC[C@H]1OC[C@H](Nc2ncccn2)[C@@H](O)[C@H]1O ; OC[C@H]1OC[C@H](Nc2nc(-c3ccncc3)ns2)[C@@H](O)[C@H]1O]

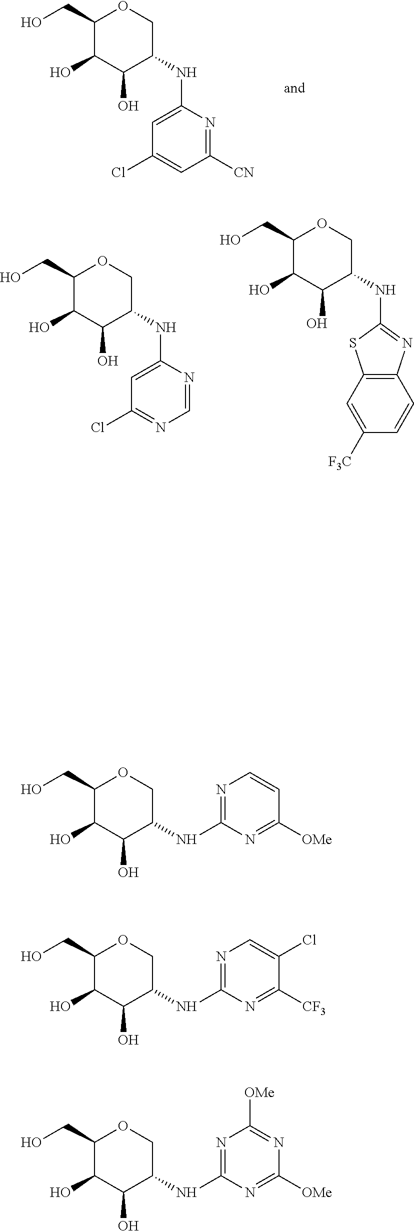 [SMILES: N#Cc1cc(Cl)cc(N[C@H]2CO[C@H](CO)[C@H](O)[C@@H]2O)n1 ; OC[C@H]1OC[C@H](Nc2cc(Cl)ncn2)[C@@H](O)[C@H]1O ; OC[C@H]1OC[C@H](Nc2nc3ccc(C(F)(F)F)cc3s2)[C@@H](O)[C@H]1O ; COc1ccnc(N[C@H]2CO[C@H](CO)[C@H](O)[C@@H]2O)n1 ; OC[C@H]1OC[C@H](Nc2ncc(Cl)c(C(F)(F)F)n2)[C@@H](O)[C@H]1O ; COc1nc(N[C@H]2CO[C@H](CO)[C@H](O)[C@@H]2O)nc(OC)n1]

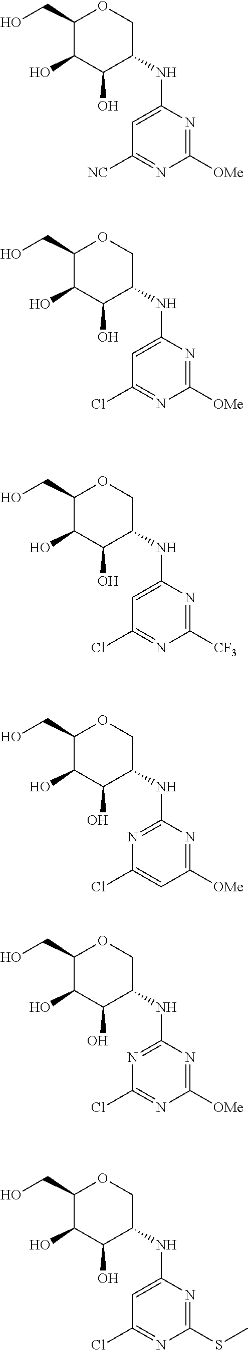 [SMILES: COc1nc(C#N)cc(N[C@H]2CO[C@H](CO)[C@H](O)[C@@H]2O)n1 ; COc1nc(Cl)cc(N[C@H]2CO[C@H](CO)[C@H](O)[C@@H]2O)n1 ; OC[C@H]1OC[C@H](Nc2cc(Cl)nc(C(F)(F)F)n2)[C@@H](O)[C@H]1O ; COc1cc(Cl)nc(N[C@H]2CO[C@H](CO)[C@H](O)[C@@H]2O)n1 ; COc1nc(Cl)nc(N[C@H]2CO[C@H](CO)[C@H](O)[C@@H]2O)n1 ; CSc1nc(Cl)cc(N[C@H]2CO[C@H](CO)[C@H](O)[C@@H]2O)n1]

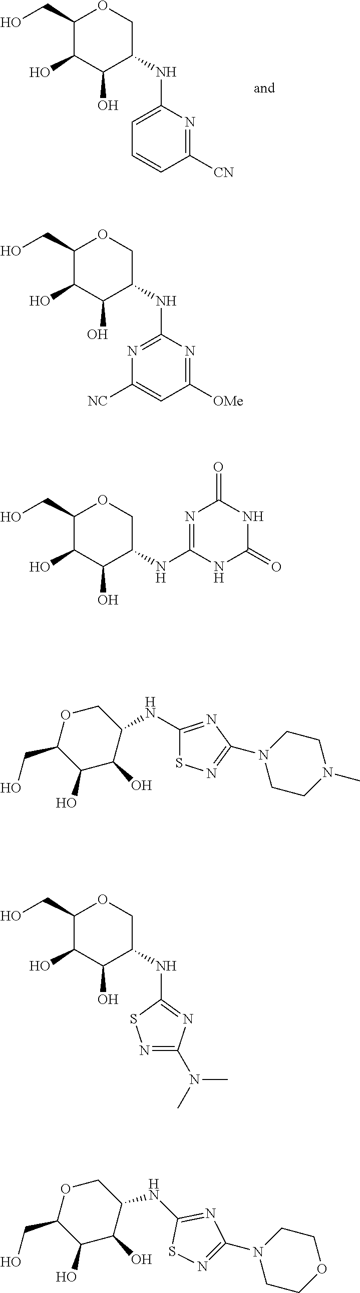 [SMILES: N#Cc1cccc(N[C@H]2CO[C@H](CO)[C@H](O)[C@@H]2O)n1 ; COc1cc(C#N)nc(N[C@H]2CO[C@H](CO)[C@H](O)[C@@H]2O)n1 ; O=c1nc(N[C@H]2CO[C@H](CO)[C@H](O)[C@@H]2O)[nH]c(=O)[nH]1 ; CN1CCN(c2nsc(N[C@H]3CO[C@H](CO)[C@H](O)[C@@H]3O)n2)CC1 ; CN(C)c1nsc(N[C@H]2CO[C@H](CO)[C@H](O)[C@@H]2O)n1 ; OC[C@H]1OC[C@H](Nc2nc(N3CCOCC3)ns2)[C@@H](O)[C@H]1O]

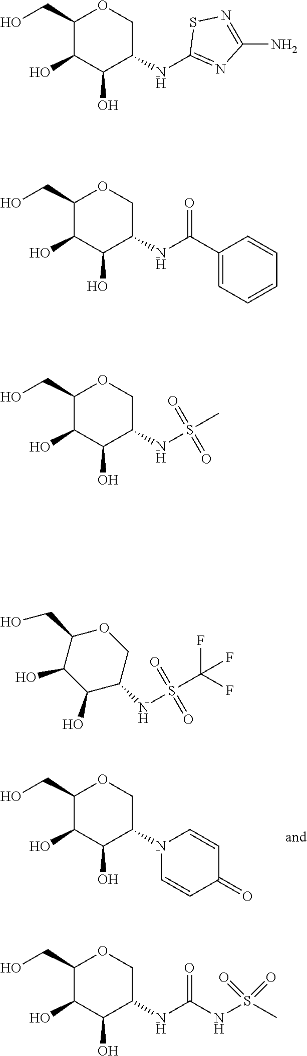 [SMILES: Nc1nsc(N[C@H]2CO[C@H](CO)[C@H](O)[C@@H]2O)n1 ; O=C(N[C@H]1CO[C@H](CO)[C@H](O)[C@@H]1O)c1ccccc1 ; CS(=O)(=O)N[C@H]1CO[C@H](CO)[C@H](O)[C@@H]1O ; O=S(=O)(N[C@H]1CO[C@H](CO)[C@H](O)[C@@H]1O)C(F)(F)F ; O=c1ccn([C@H]2CO[C@H](CO)[C@H](O)[C@@H]2O)cc1 ; CS(=O)(=O)NC(=O)N[C@H]1CO[C@H](CO)[C@H](O)[C@@H]1O]

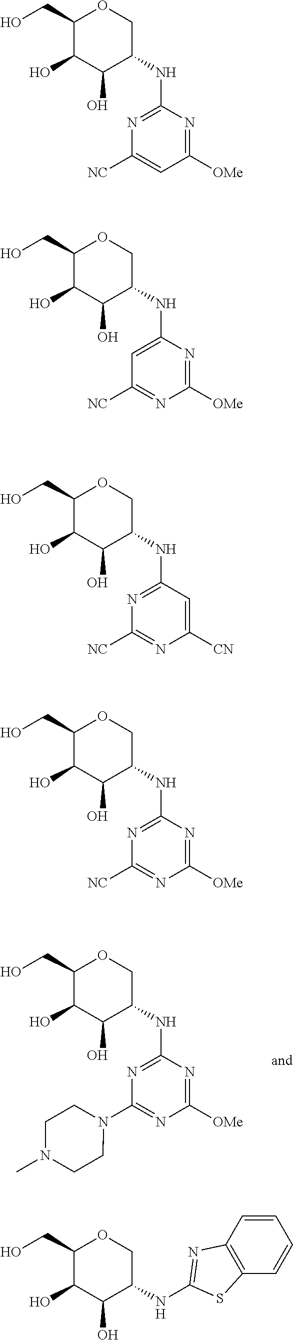 [SMILES: COc1cc(C#N)nc(N[C@H]2CO[C@H](CO)[C@H](O)[C@@H]2O)n1 ; COc1nc(C#N)cc(N[C@H]2CO[C@H](CO)[C@H](O)[C@@H]2O)n1 ; N#Cc1cc(N[C@H]2CO[C@H](CO)[C@H](O)[C@@H]2O)nc(C#N)n1 ; COc1nc(C#N)nc(N[C@H]2CO[C@H](CO)[C@H](O)[C@@H]2O)n1 ; COc1nc(N[C@H]2CO[C@H](CO)[C@H](O)[C@@H]2O)nc(N2CCN(C)CC2)n1 ; OC[C@H]1OC[C@H](Nc2nc3ccccc3s2)[C@@H](O)[C@H]1O]

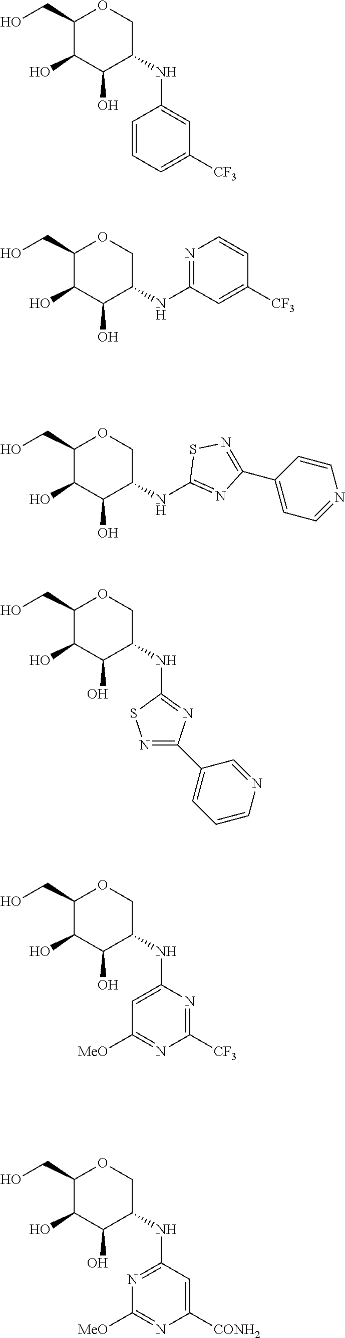 [SMILES: OC[C@H]1OC[C@H](Nc2cccc(C(F)(F)F)c2)[C@@H](O)[C@H]1O ; OC[C@H]1OC[C@H](Nc2cc(C(F)(F)F)ccn2)[C@@H](O)[C@H]1O ; OC[C@H]1OC[C@H](Nc2nc(-c3ccncc3)ns2)[C@@H](O)[C@H]1O ; OC[C@H]1OC[C@H](Nc2nc(-c3cccnc3)ns2)[C@@H](O)[C@H]1O ; COc1cc(N[C@H]2CO[C@H](CO)[C@H](O)[C@@H]2O)nc(C(F)(F)F)n1 ; COc1nc(N[C@H]2CO[C@H](CO)[C@H](O)[C@@H]2O)cc(C(N)=O)n1]

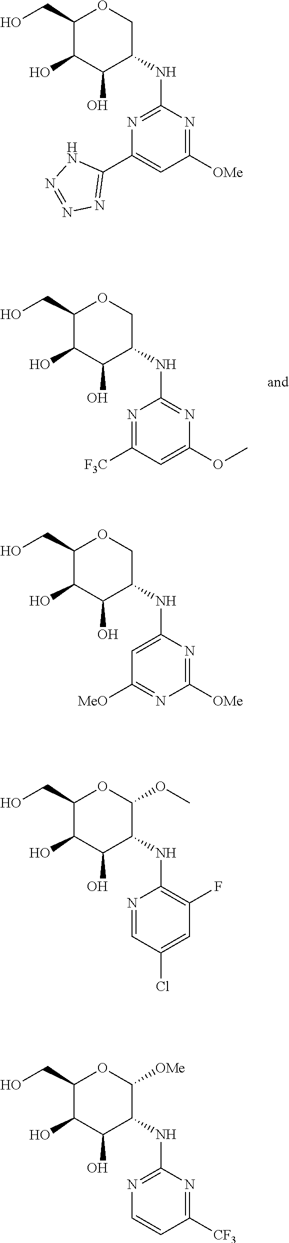 [SMILES: COc1cc(-c2nnn[nH]2)nc(N[C@H]2CO[C@H](CO)[C@H](O)[C@@H]2O)n1 ; COc1cc(C(F)(F)F)nc(N[C@H]2CO[C@H](CO)[C@H](O)[C@@H]2O)n1 ; COc1cc(N[C@H]2CO[C@H](CO)[C@H](O)[C@@H]2O)nc(OC)n1 ; CO[C@H]1O[C@H](CO)[C@H](O)[C@H](O)[C@H]1Nc1ncc(Cl)cc1F ; CO[C@H]1O[C@H](CO)[C@H](O)[C@H](O)[C@H]1Nc1nccc(C(F)(F)F)n1]

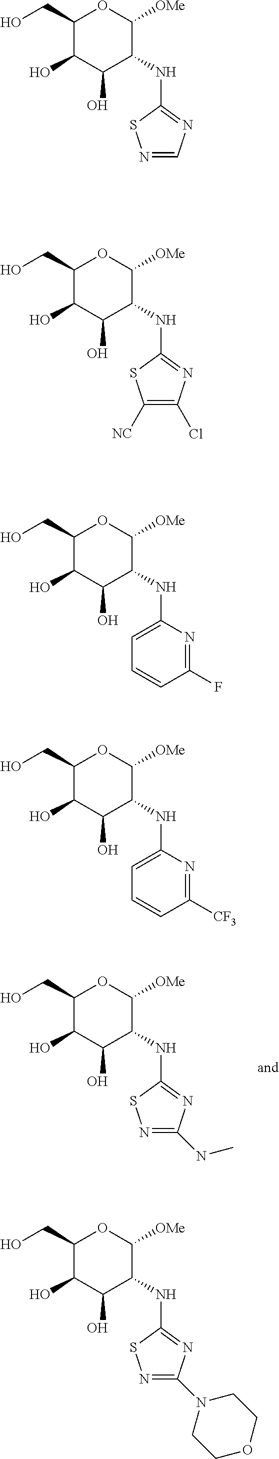 [SMILES: CO[C@H]1O[C@H](CO)[C@H](O)[C@H](O)[C@H]1Nc1ncns1 ; CO[C@H]1O[C@H](CO)[C@H](O)[C@H](O)[C@H]1Nc1nc(Cl)c(C#N)s1 ; CO[C@H]1O[C@H](CO)[C@H](O)[C@H](O)[C@H]1Nc1cccc(F)n1 ; CO[C@H]1O[C@H](CO)[C@H](O)[C@H](O)[C@H]1Nc1cccc(C(F)(F)F)n1 ; CNc1nsc(N[C@H]2[C@@H](OC)O[C@H](CO)[C@H](O)[C@@H]2O)n1 ; CO[C@H]1O[C@H](CO)[C@H](O)[C@H](O)[C@H]1Nc1nc(N2CCOCC2)ns1]

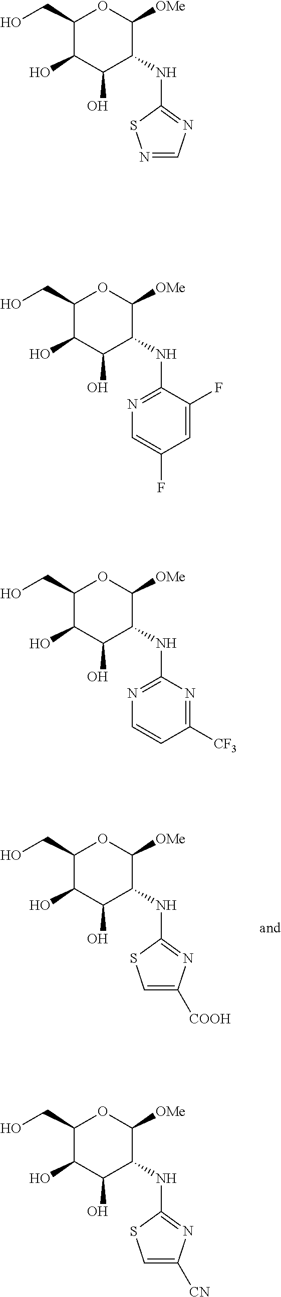 [SMILES: CO[C@@H]1O[C@H](CO)[C@H](O)[C@H](O)[C@H]1Nc1ncns1 ; CO[C@@H]1O[C@H](CO)[C@H](O)[C@H](O)[C@H]1Nc1ncc(F)cc1F ; CO[C@@H]1O[C@H](CO)[C@H](O)[C@H](O)[C@H]1Nc1nccc(C(F)(F)F)n1 ; CO[C@@H]1O[C@H](CO)[C@H](O)[C@H](O)[C@H]1Nc1nc(C(=O)O)cs1 ; CO[C@@H]1O[C@H](CO)[C@H](O)[C@H](O)[C@H]1Nc1nc(C#N)cs1]

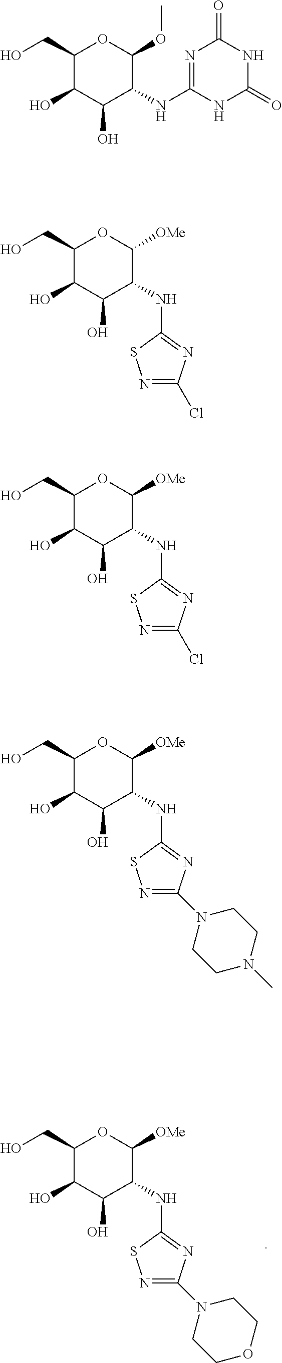 [SMILES: CO[C@@H]1O[C@H](CO)[C@H](O)[C@H](O)[C@H]1Nc1nc(=O)[nH]c(=O)[nH]1 ; CO[C@H]1O[C@H](CO)[C@H](O)[C@H](O)[C@H]1Nc1nc(Cl)ns1 ; CO[C@@H]1O[C@H](CO)[C@H](O)[C@H](O)[C@H]1Nc1nc(Cl)ns1 ; CO[C@@H]1O[C@H](CO)[C@H](O)[C@H](O)[C@H]1Nc1nc(N2CCN(C)CC2)ns1 ; CO[C@@H]1O[C@H](CO)[C@H](O)[C@H](O)[C@H]1Nc1nc(N2CCOCC2)ns1]

In certain embodiments, the compound of Formula II is selected from:

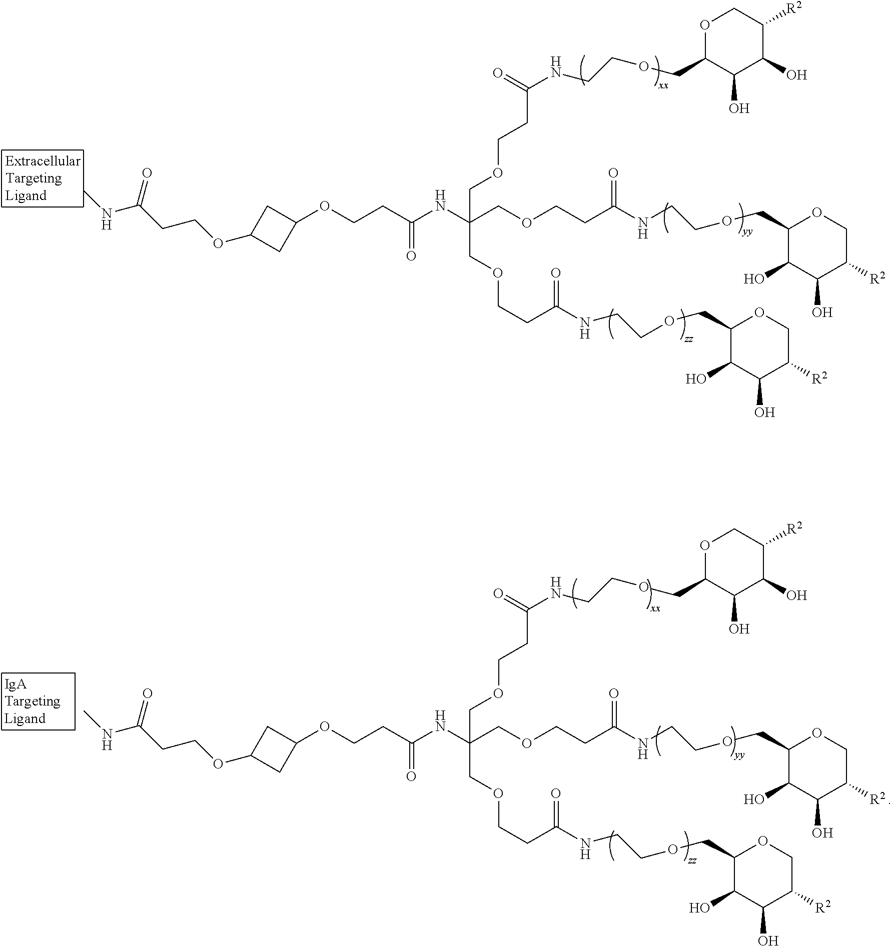

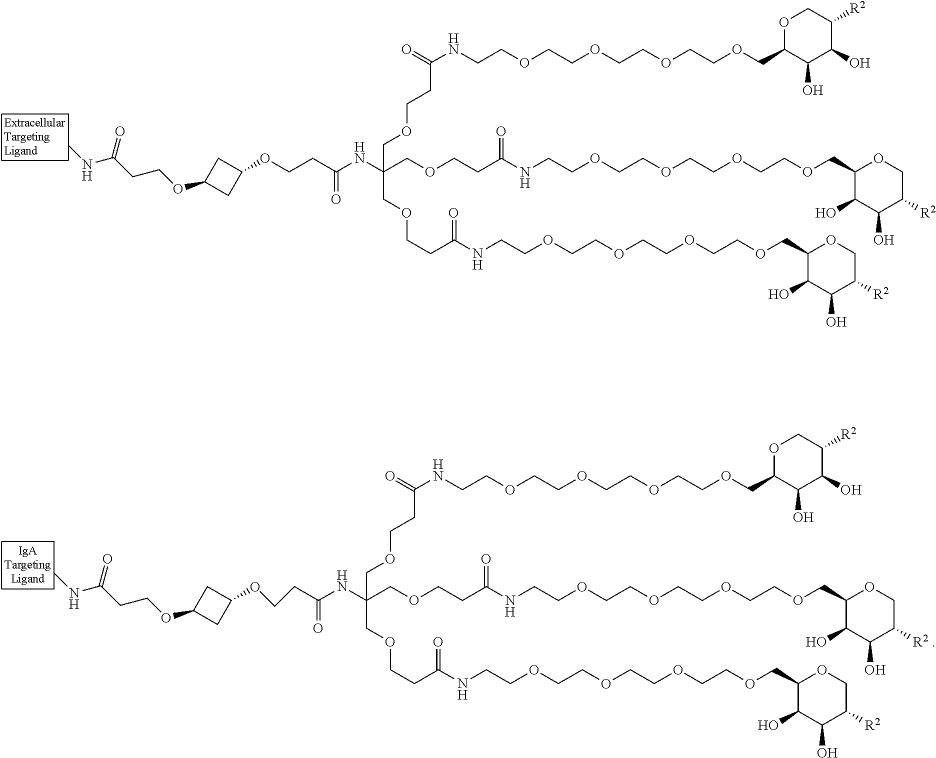

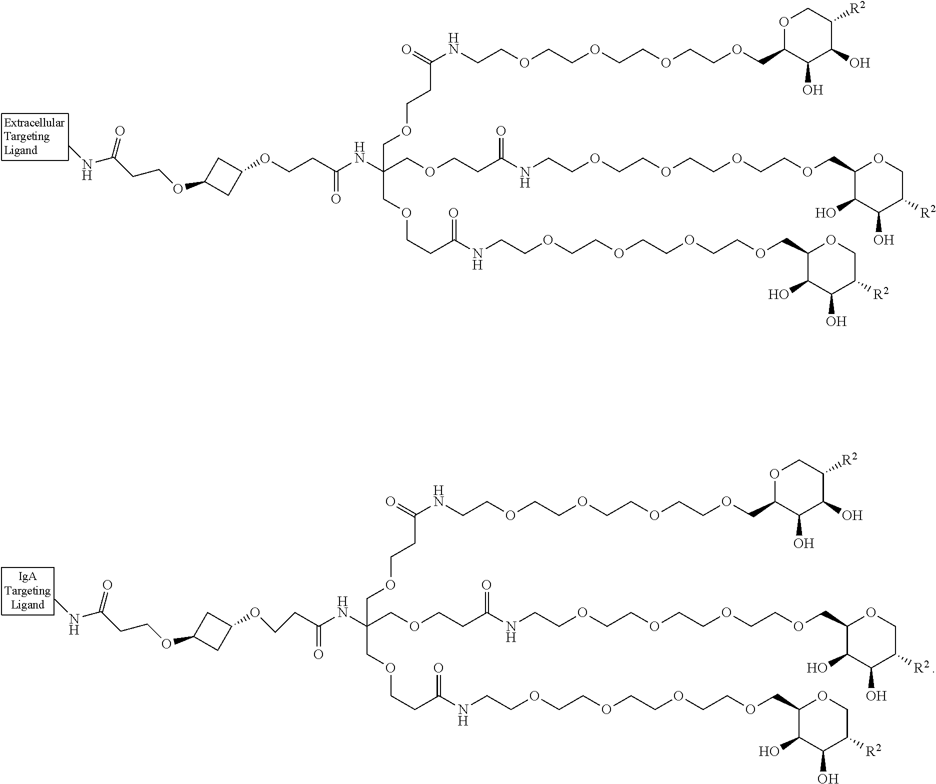

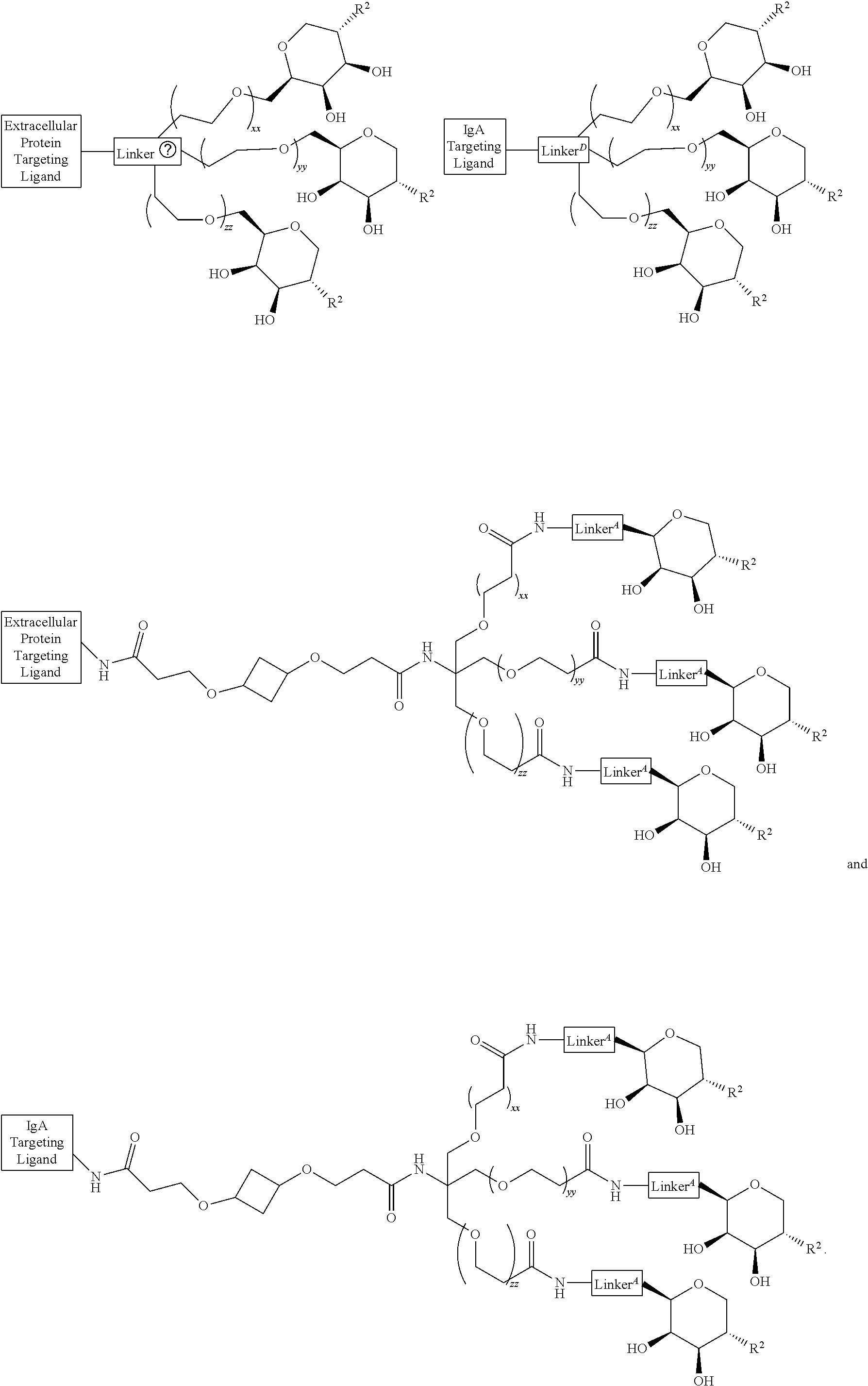

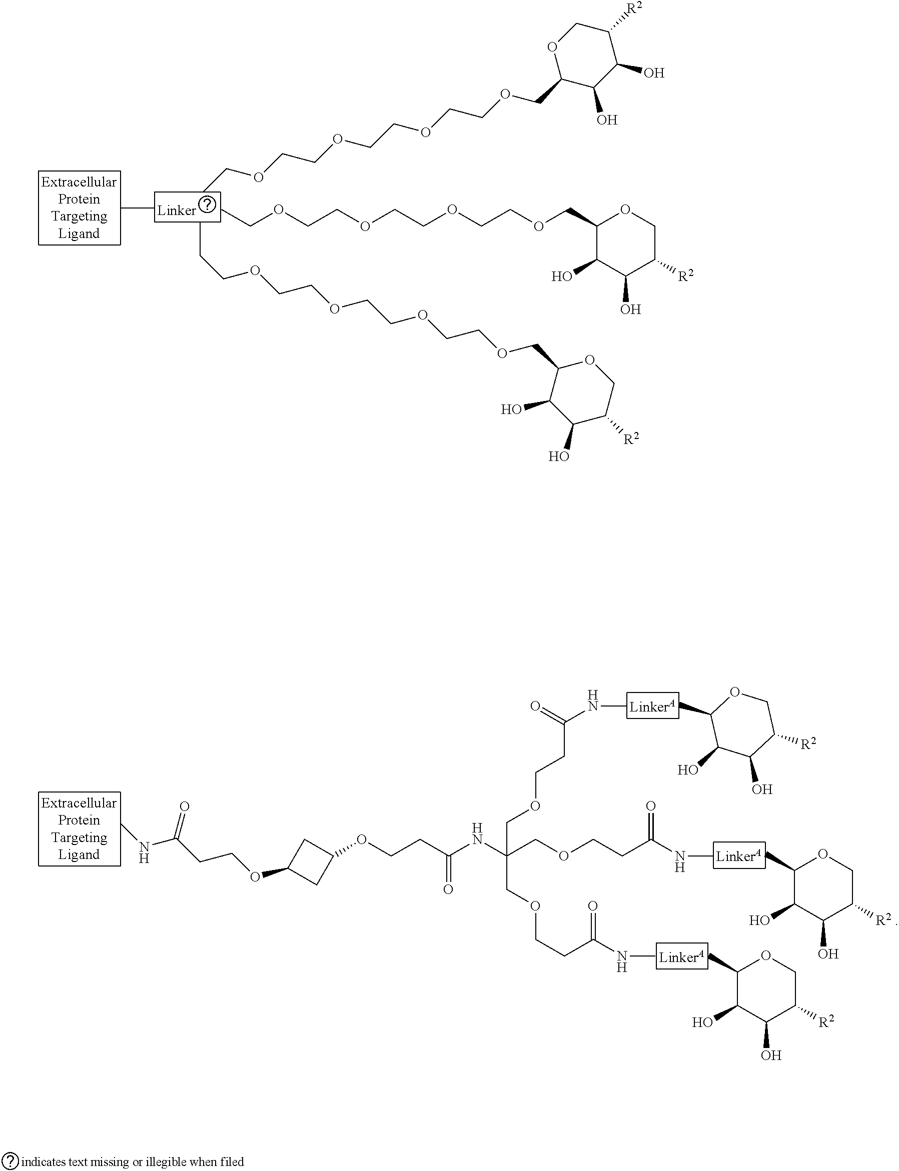

B. Talose-Based ASGPR-Binding Cellular Receptor Binding Moieties of Formula II

In certain embodiments, the compound of Formula II is selected from:

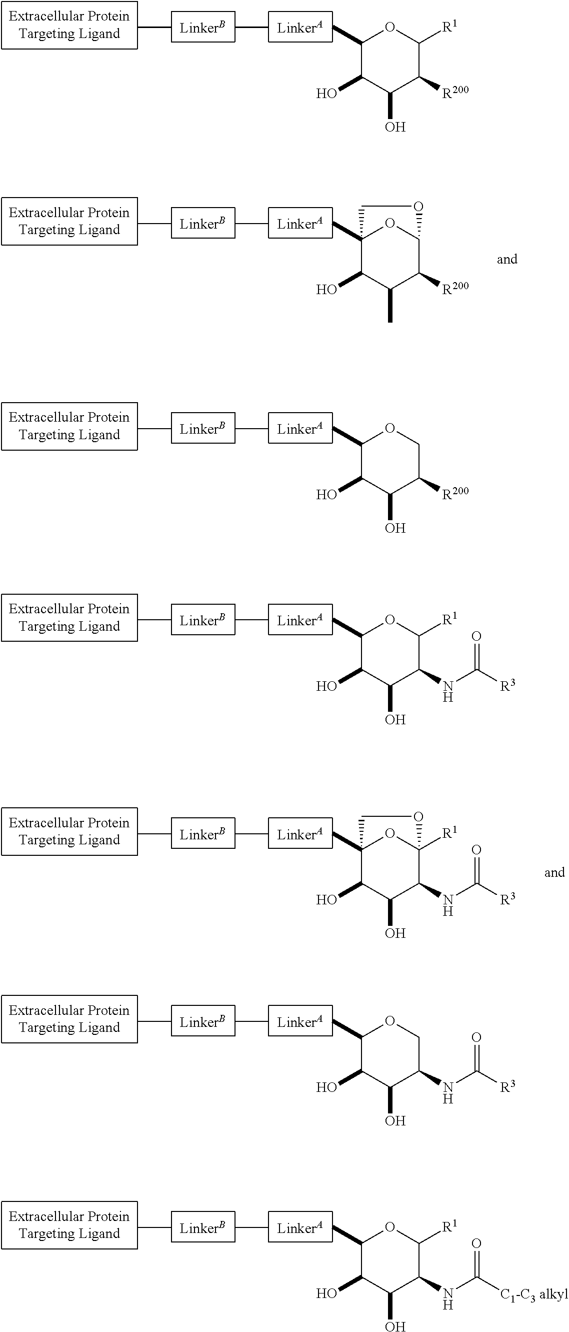

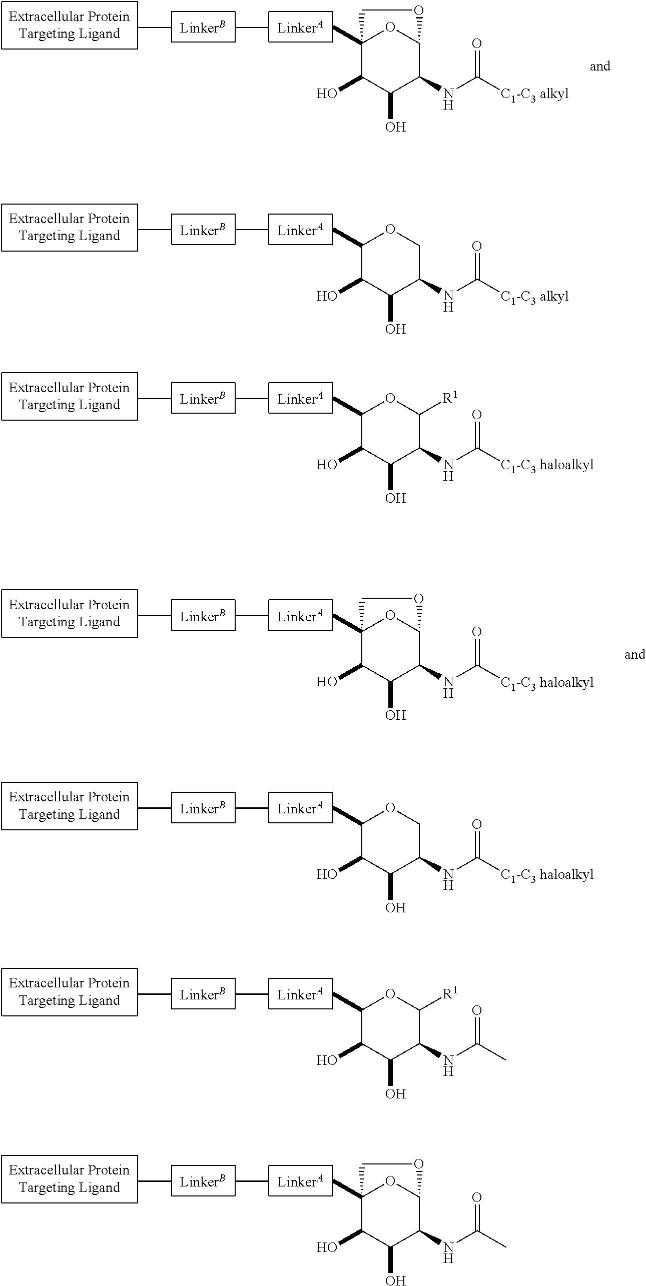

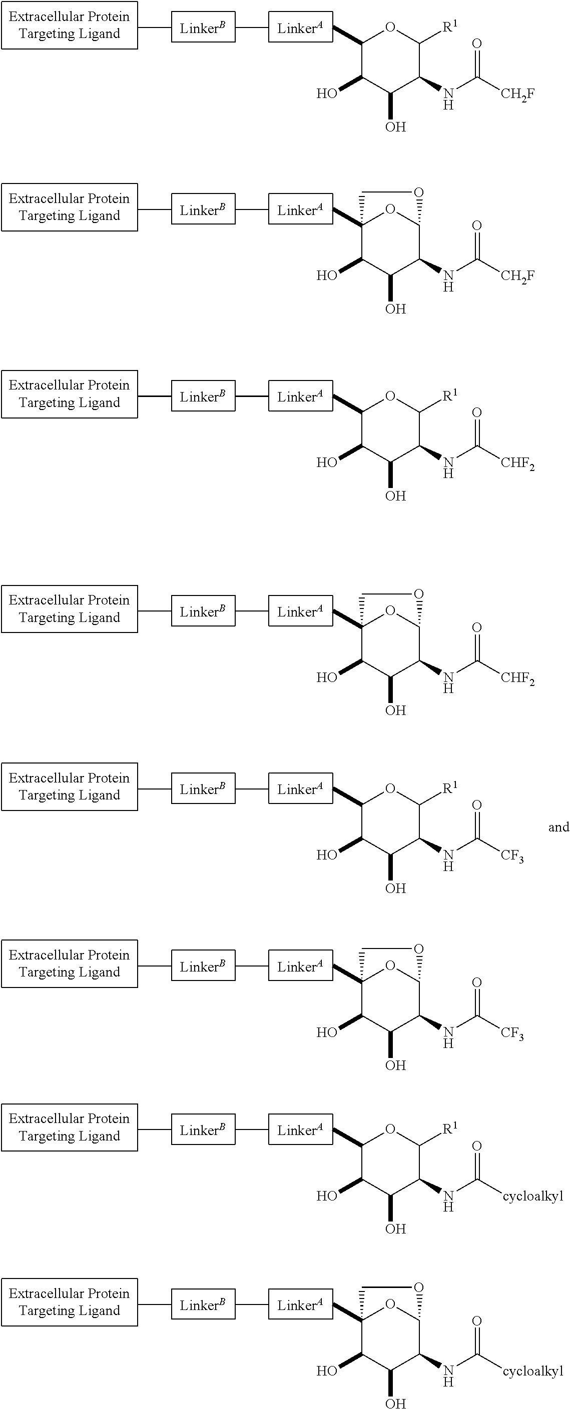

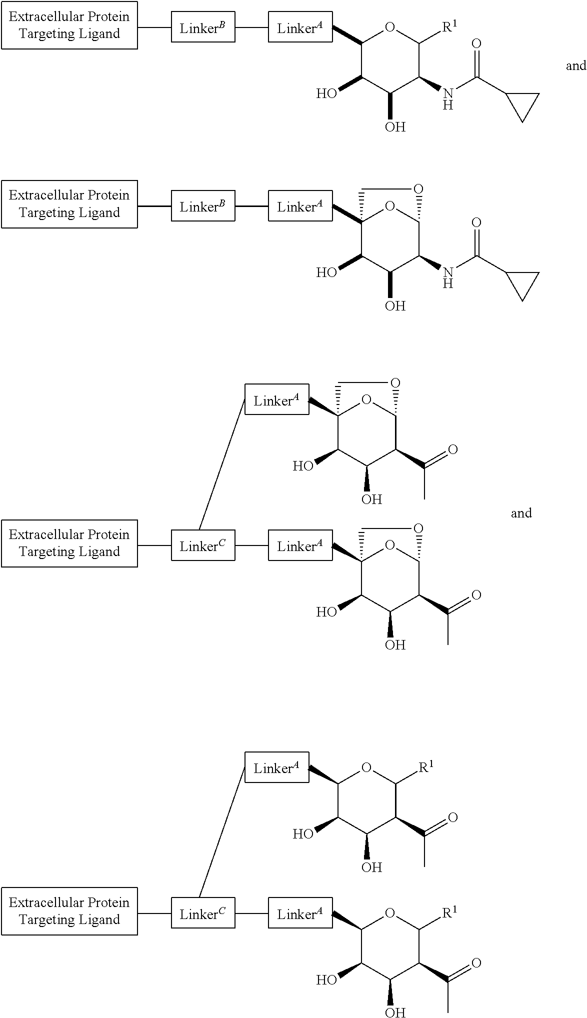

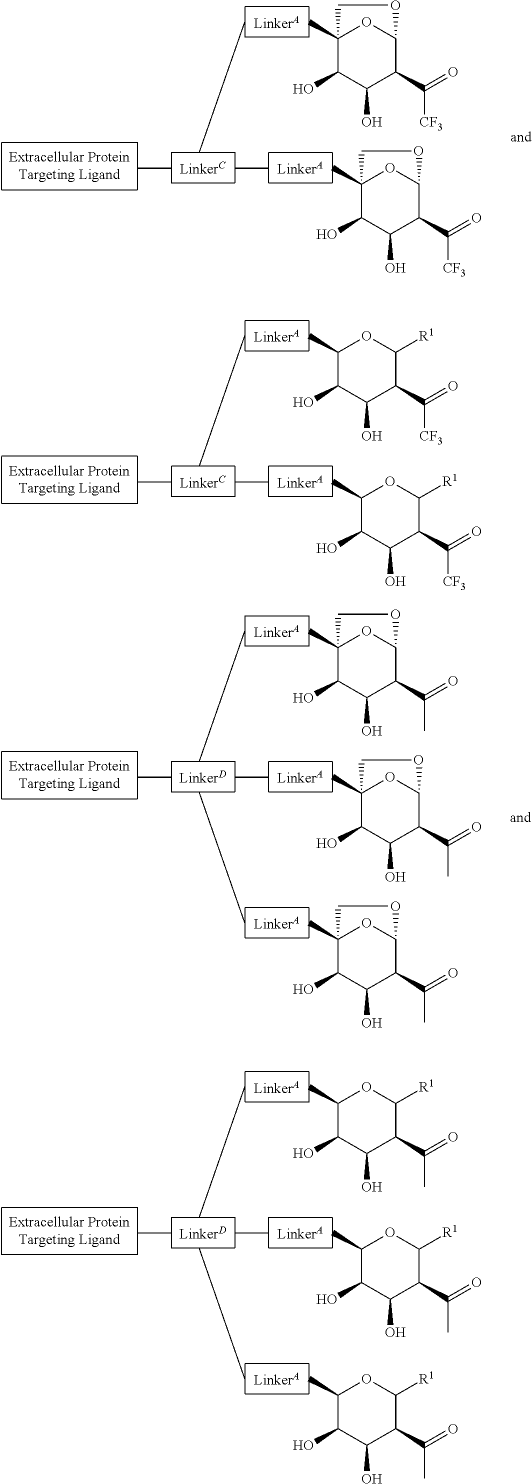

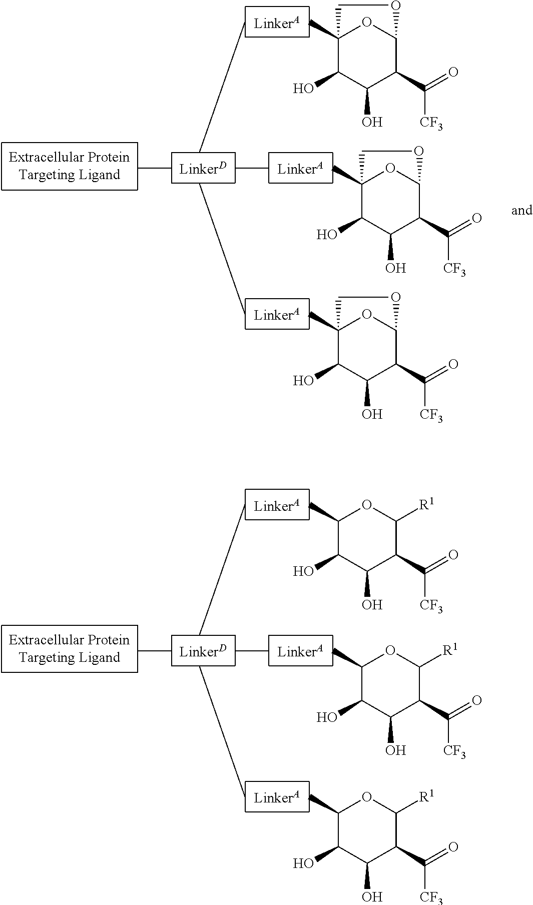

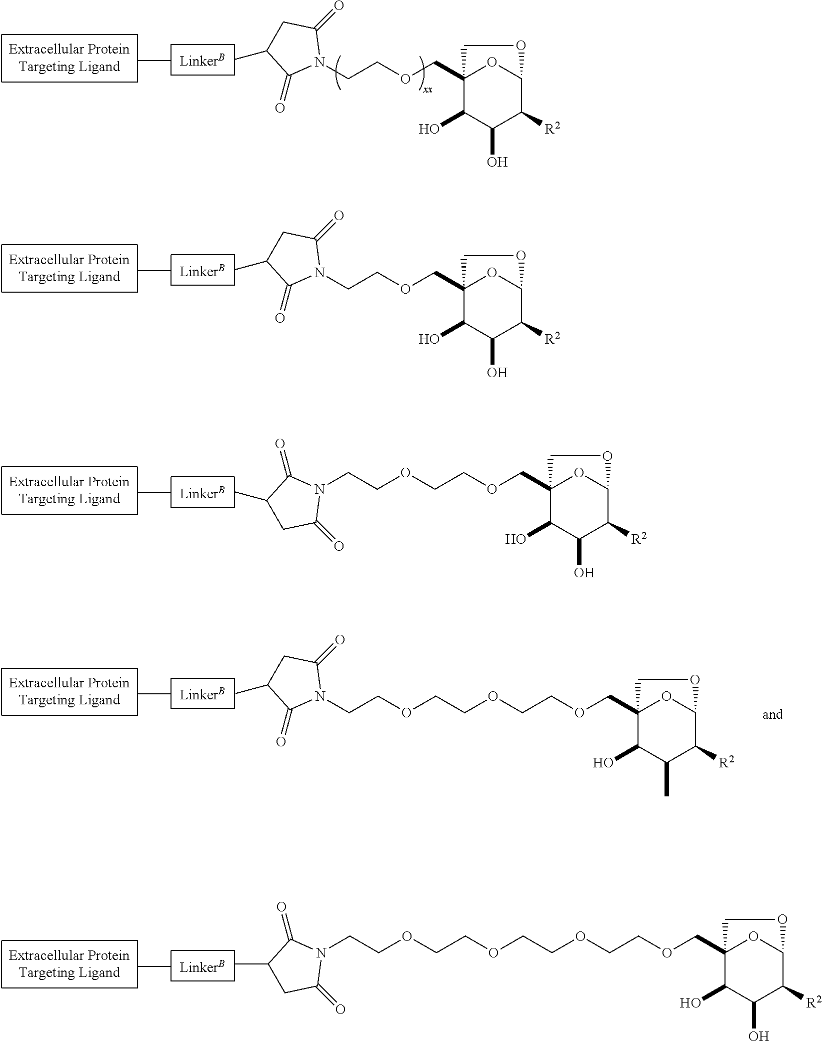

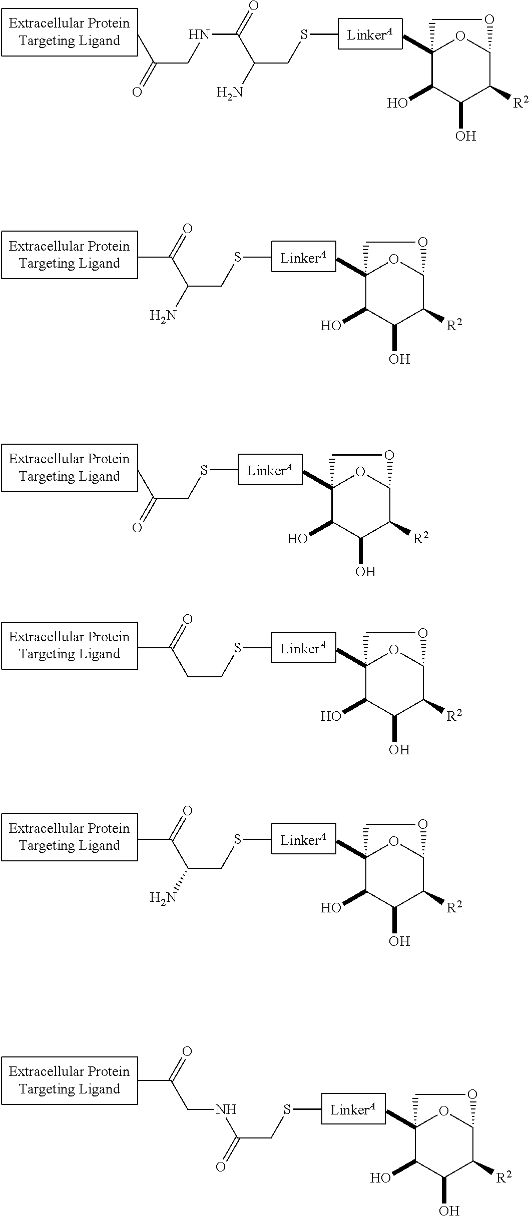

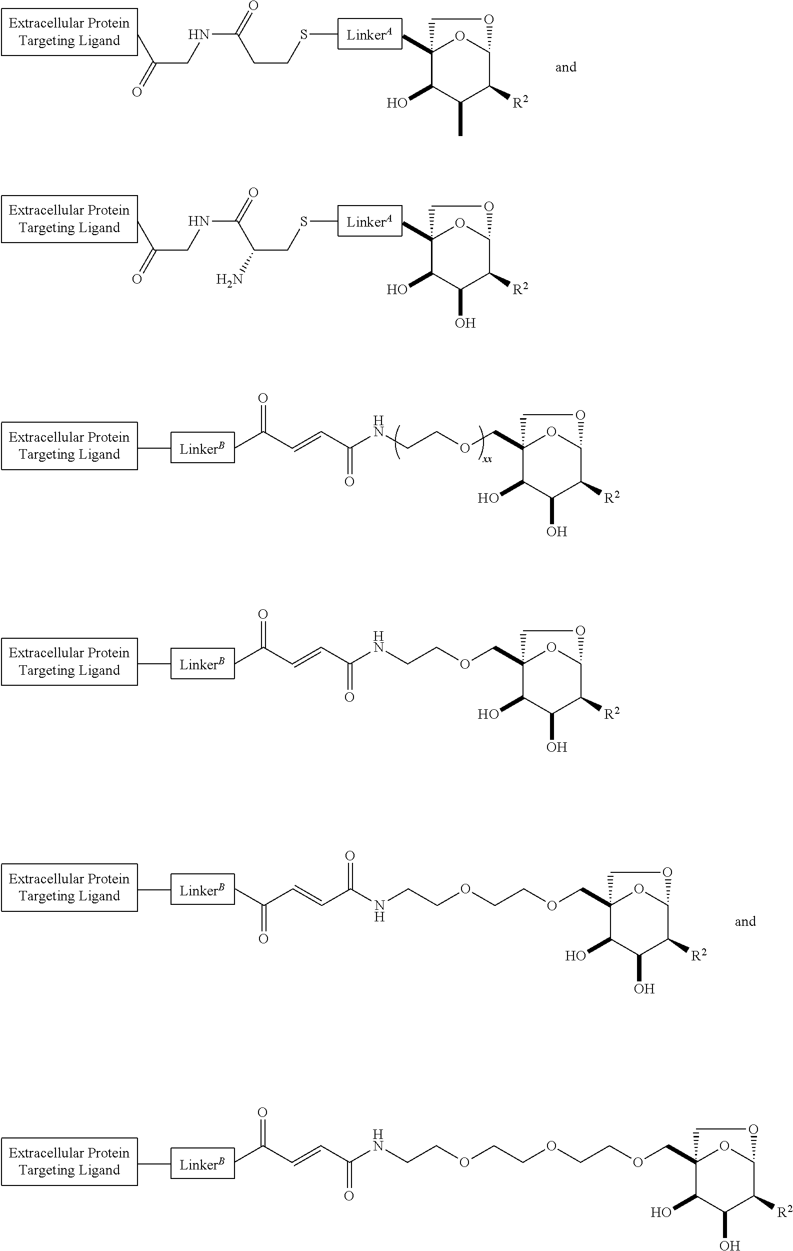

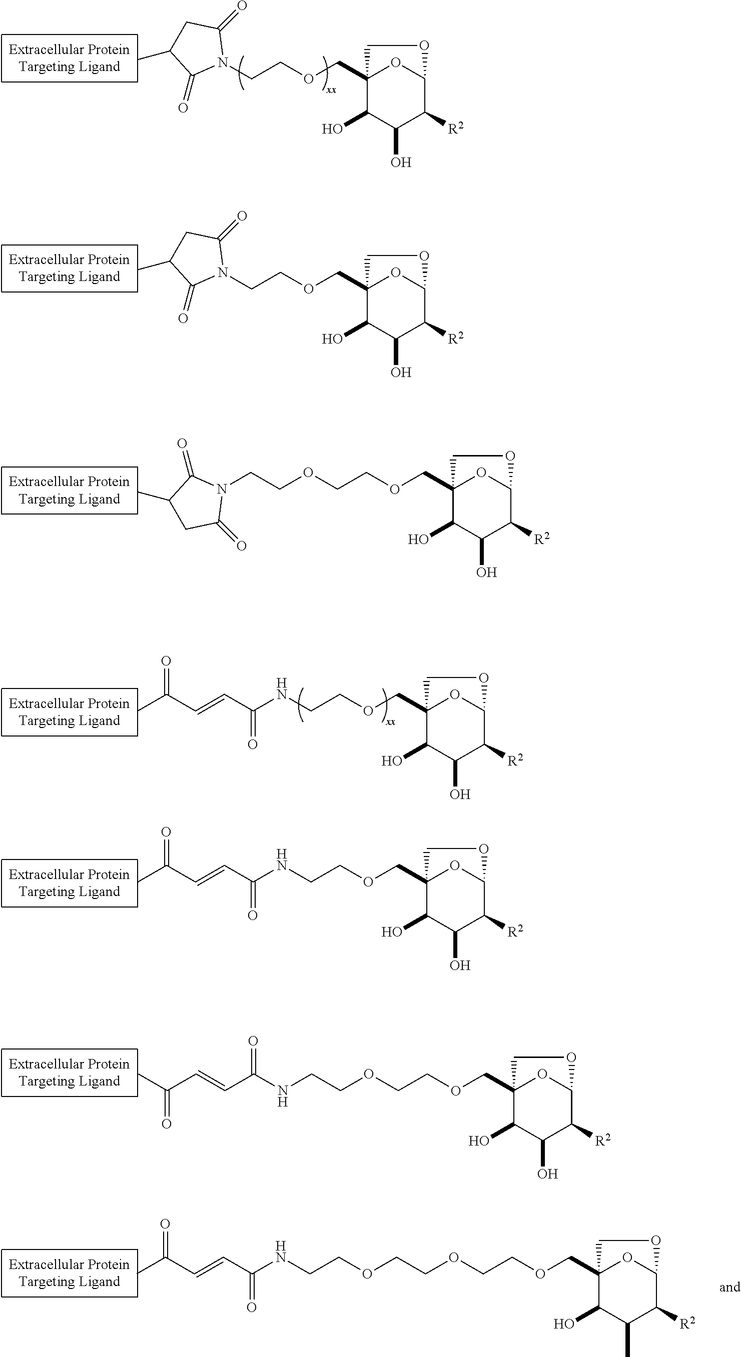

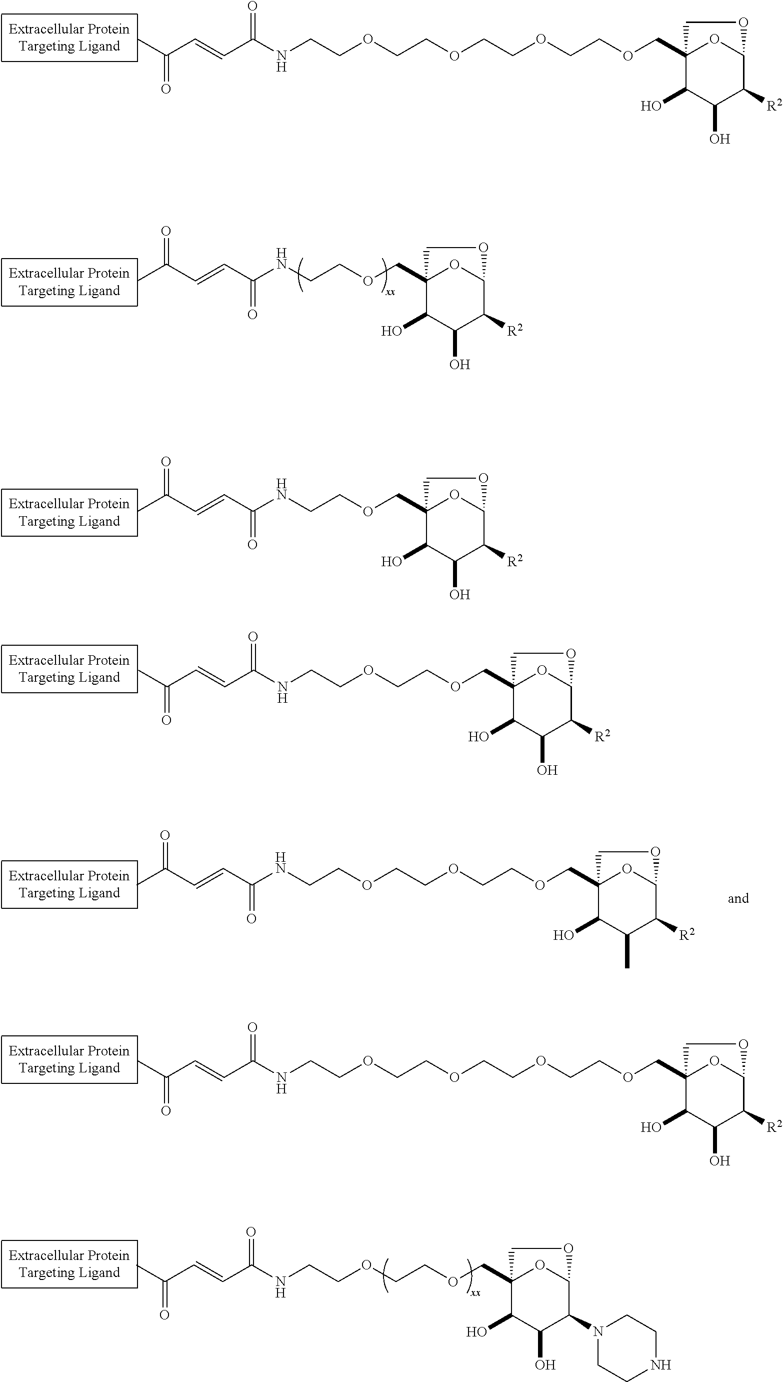

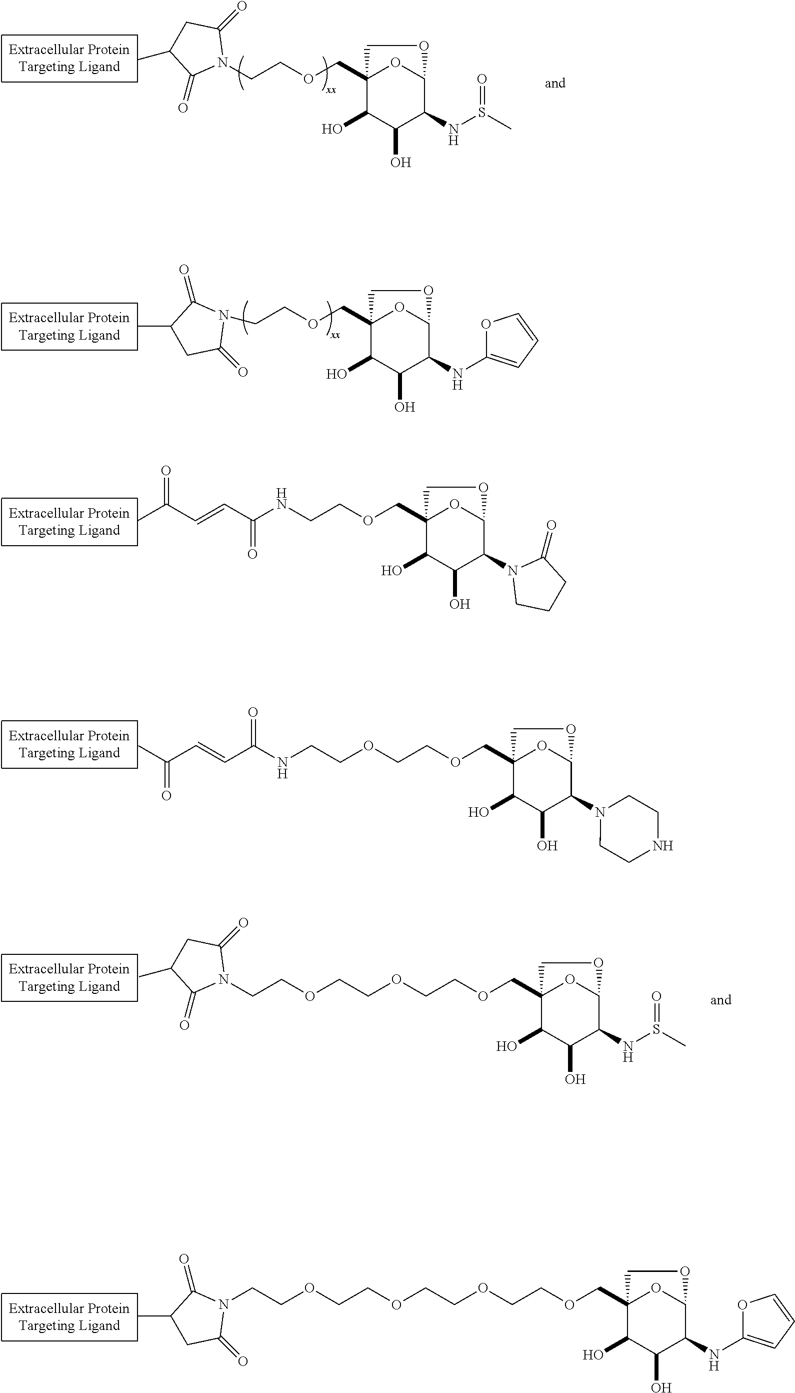

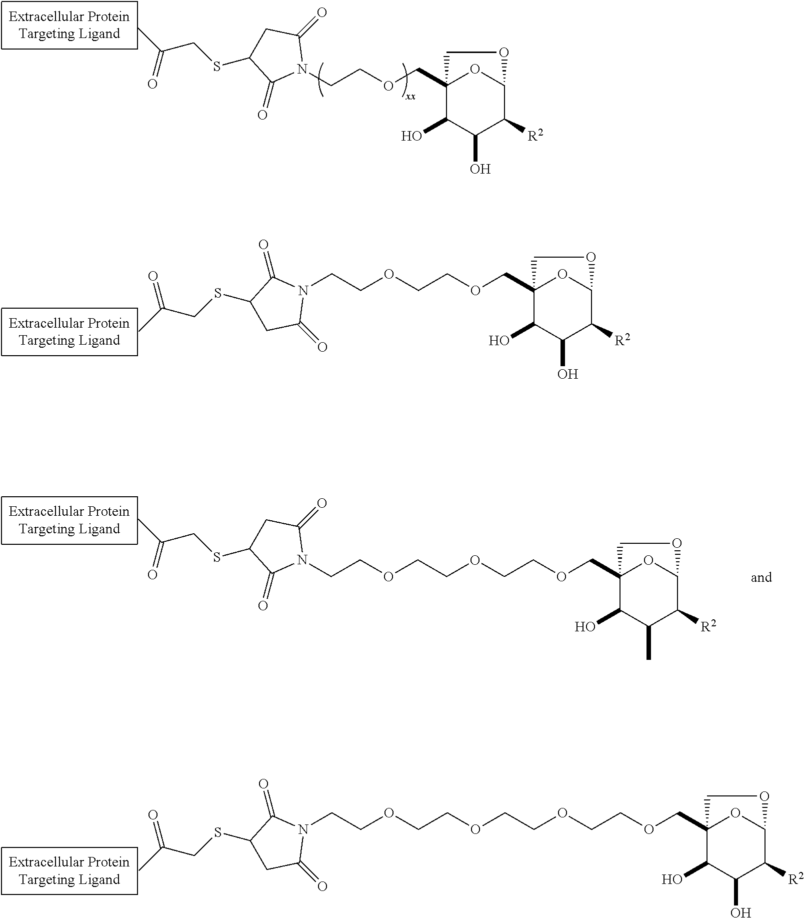

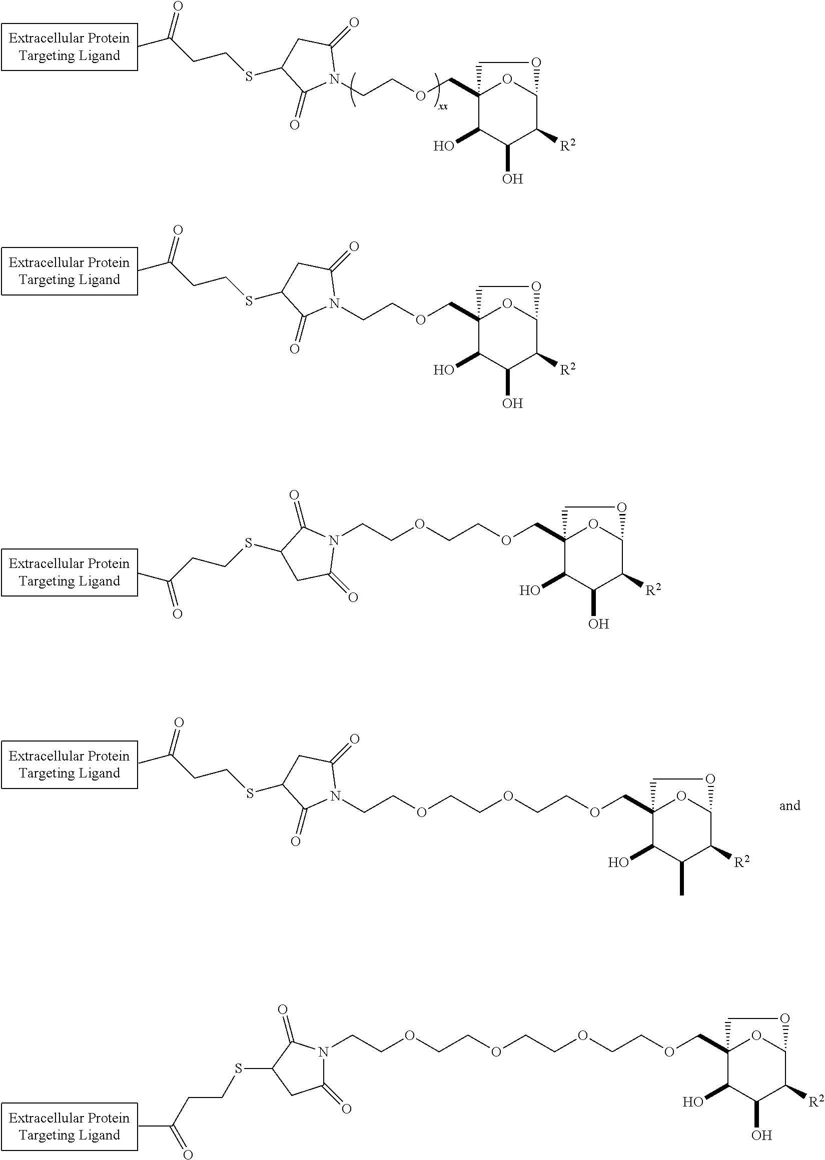

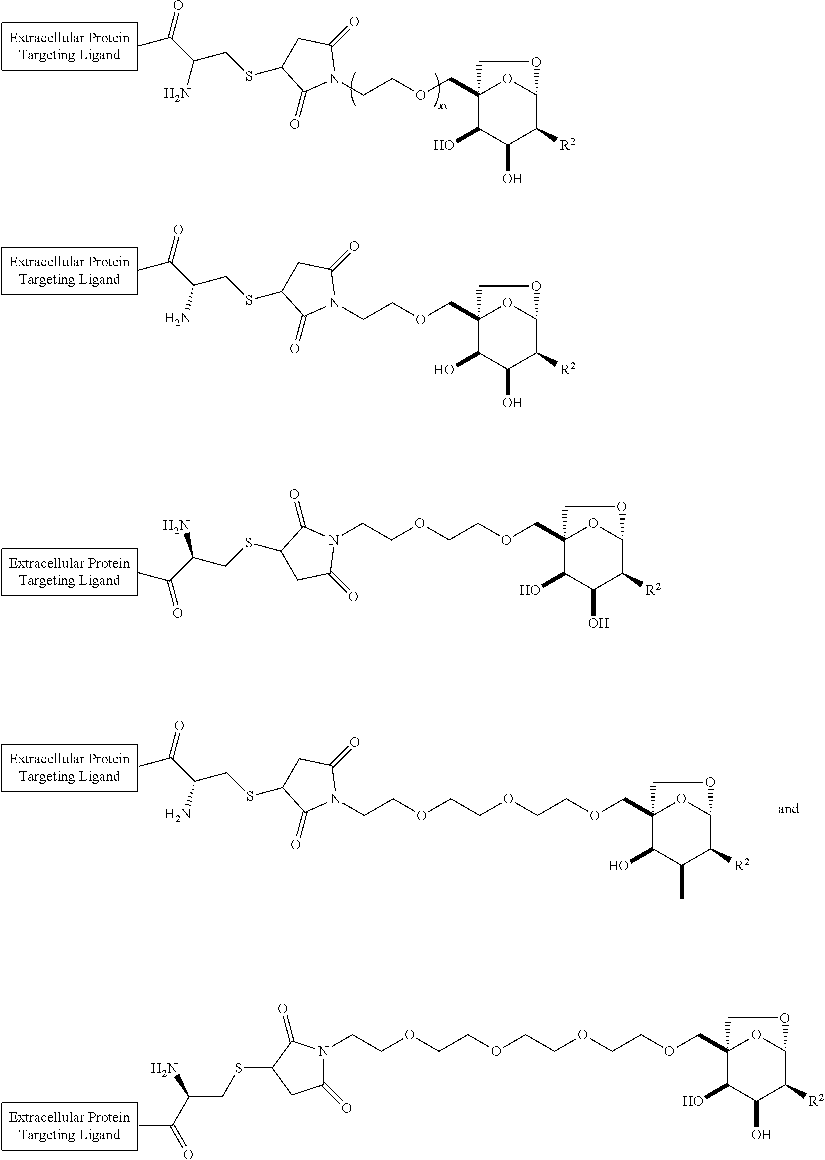

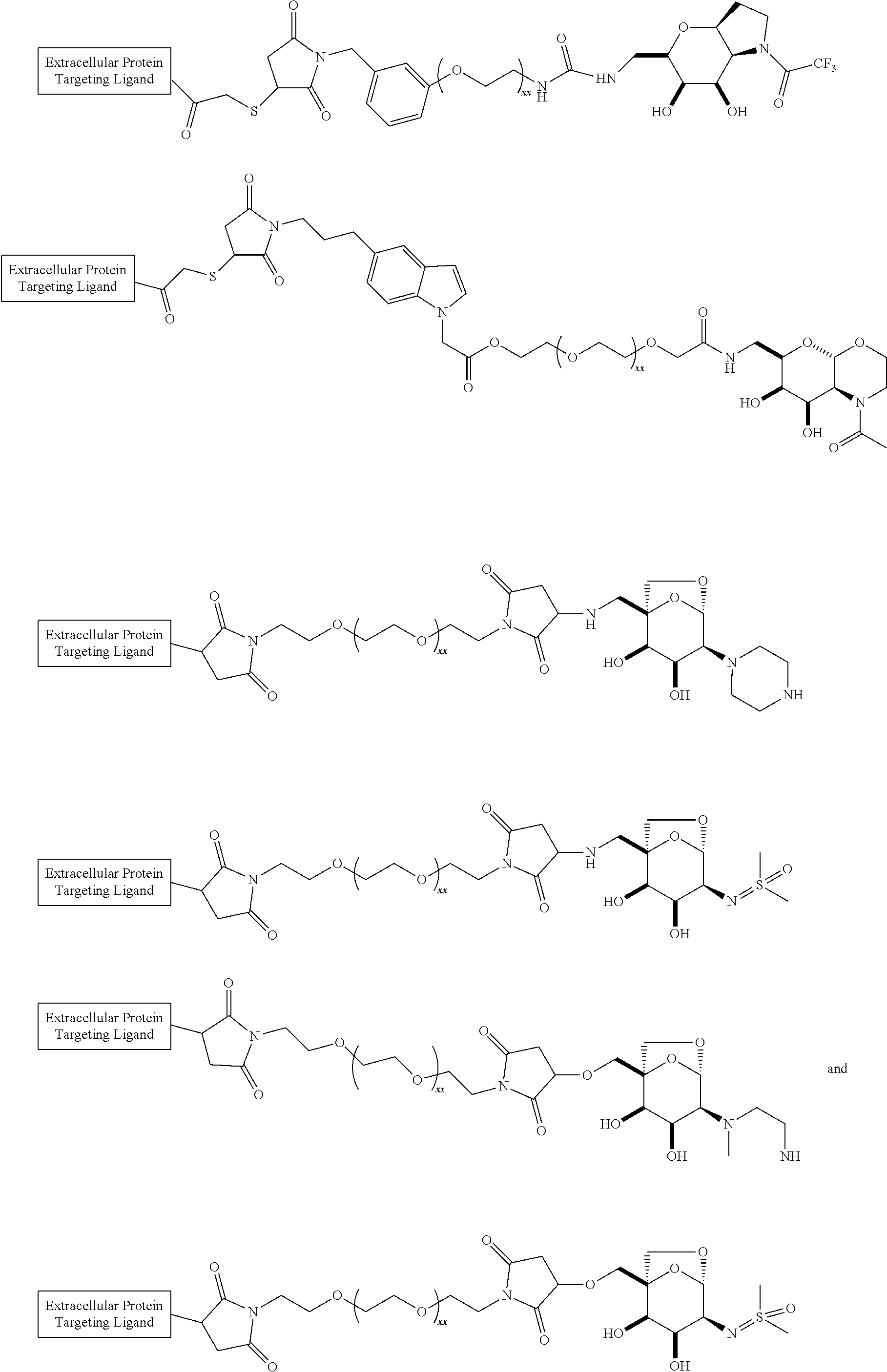

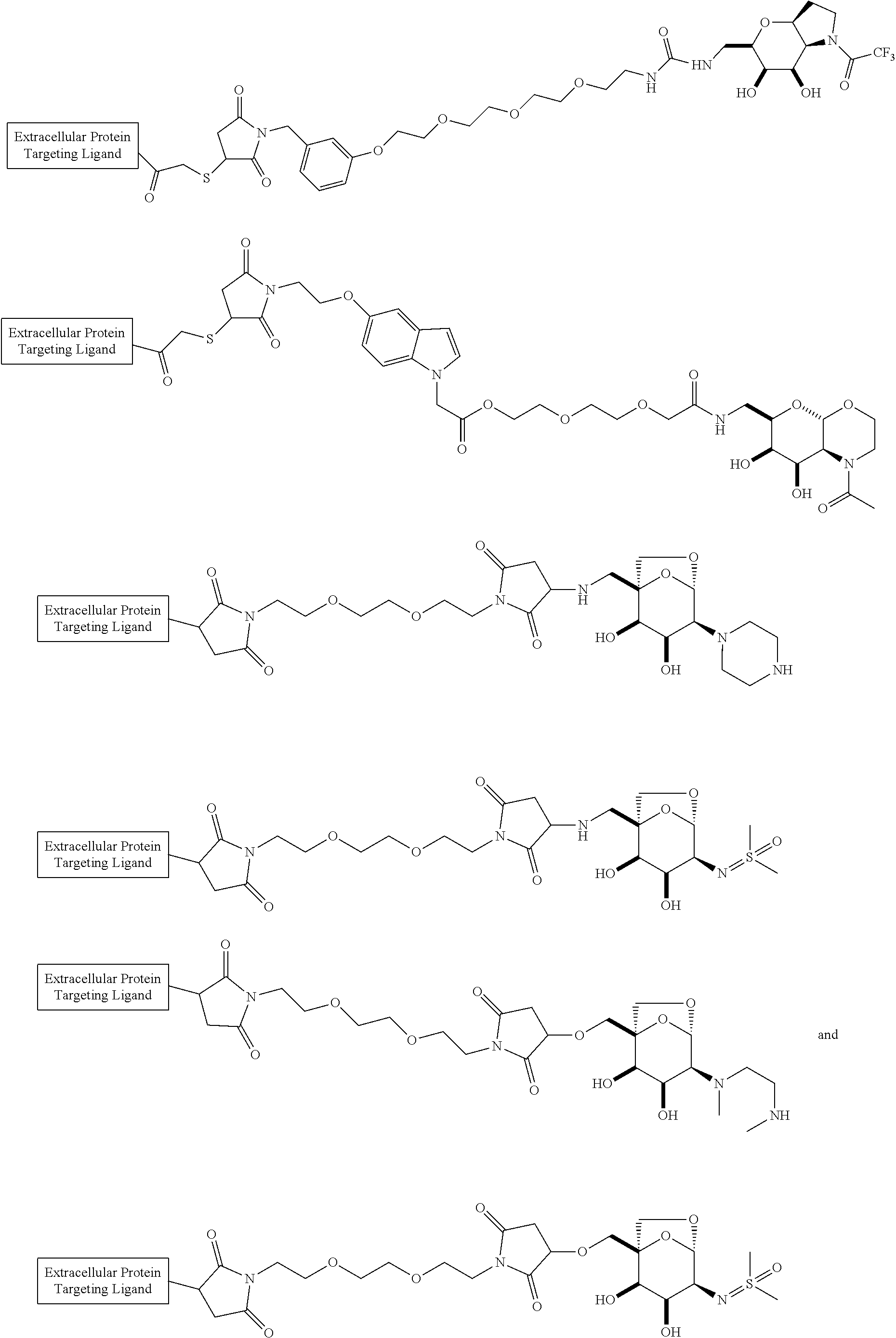

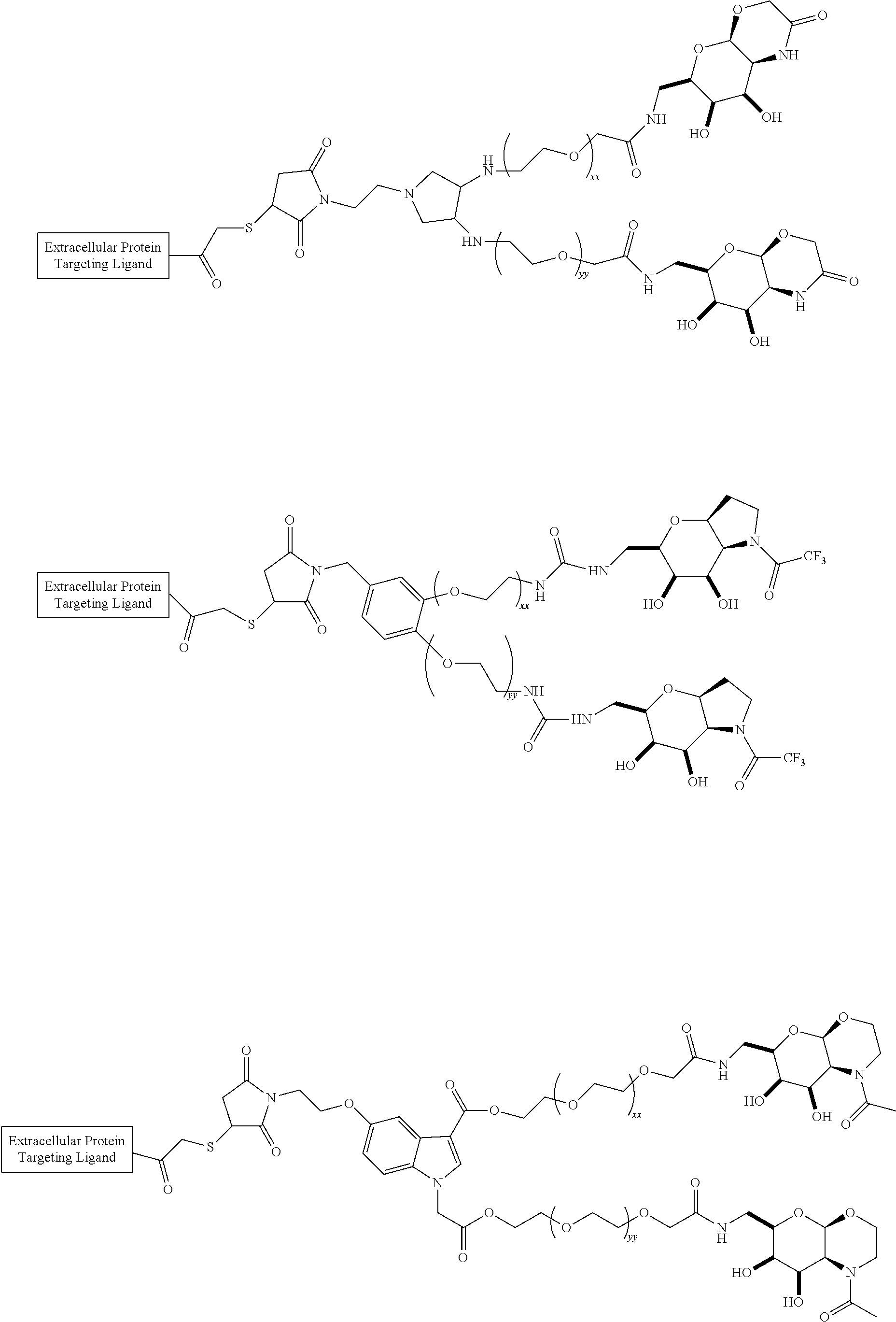

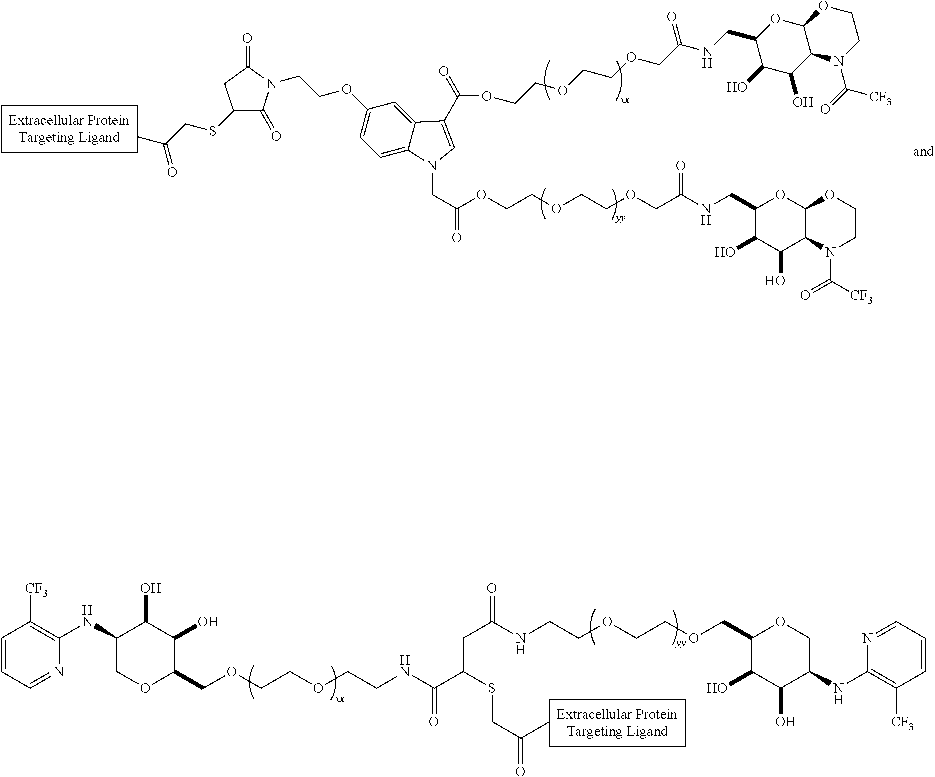

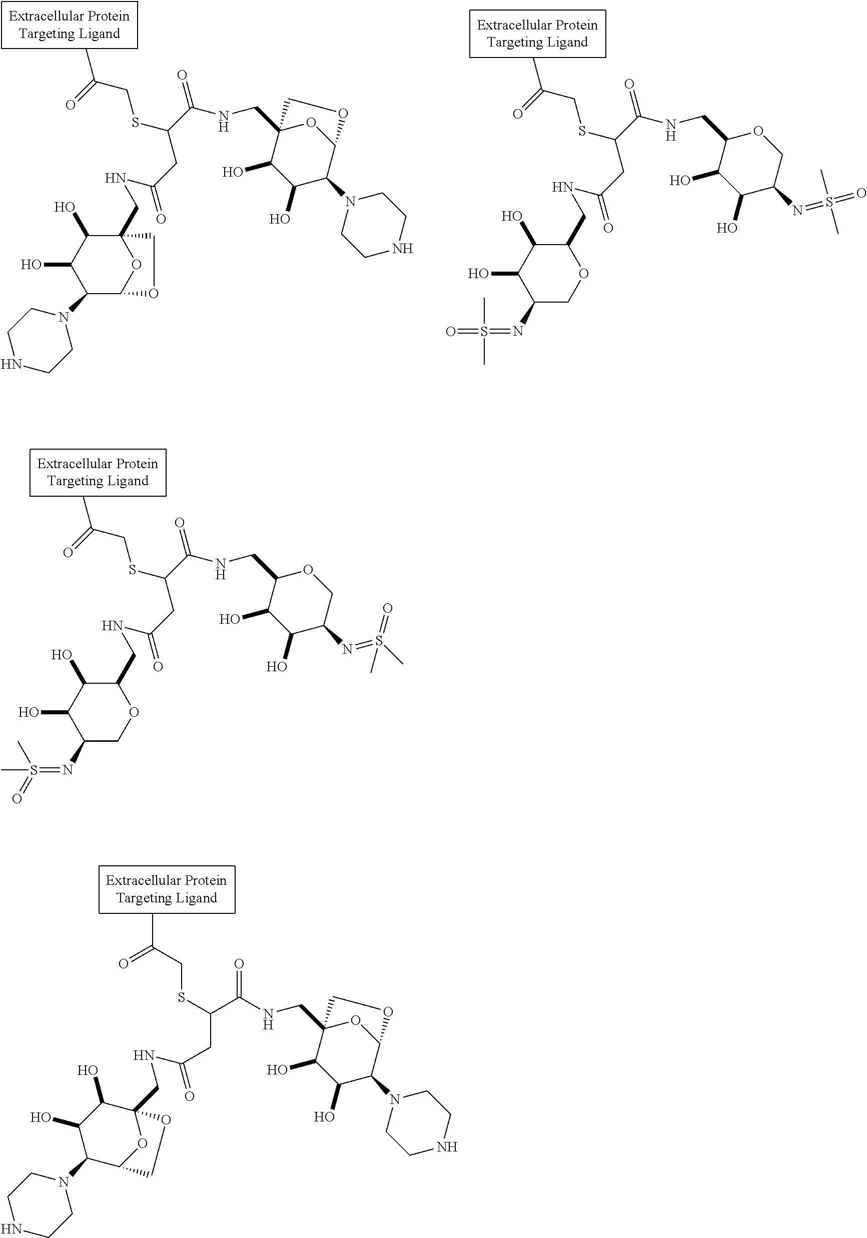

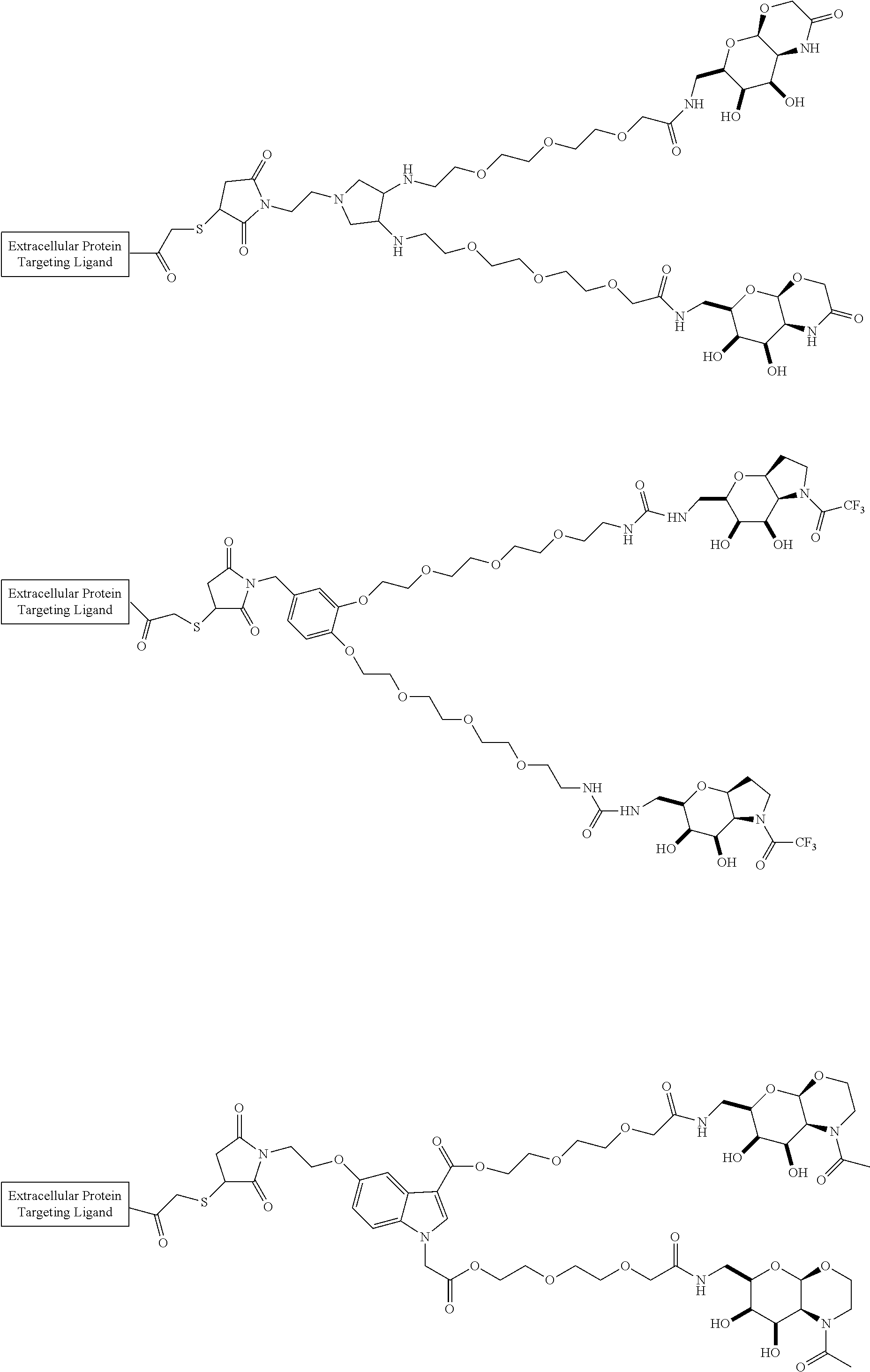

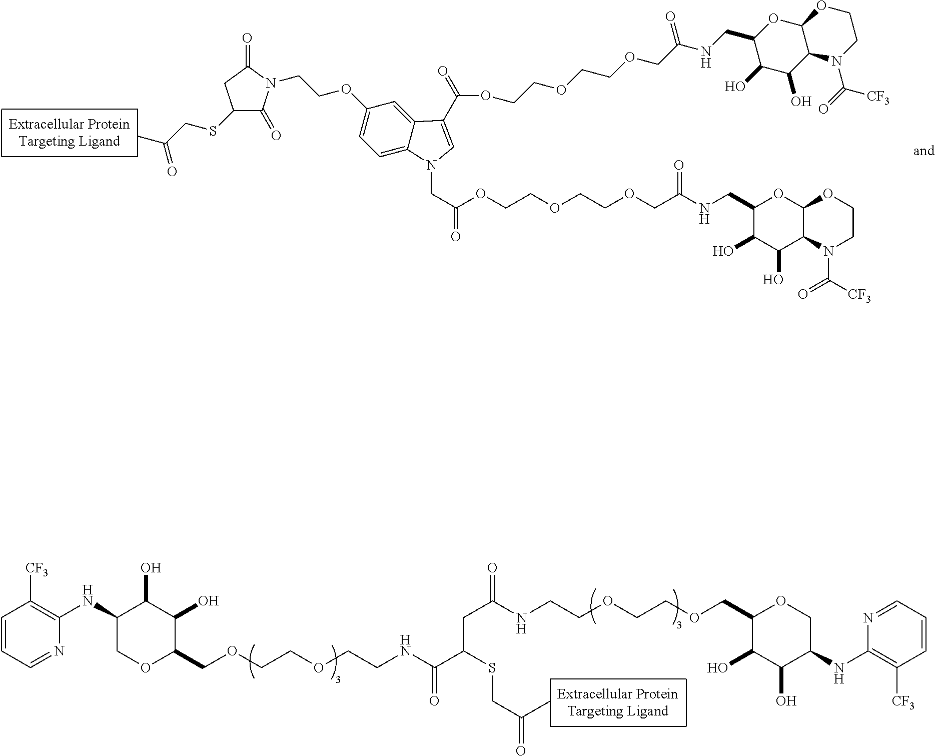

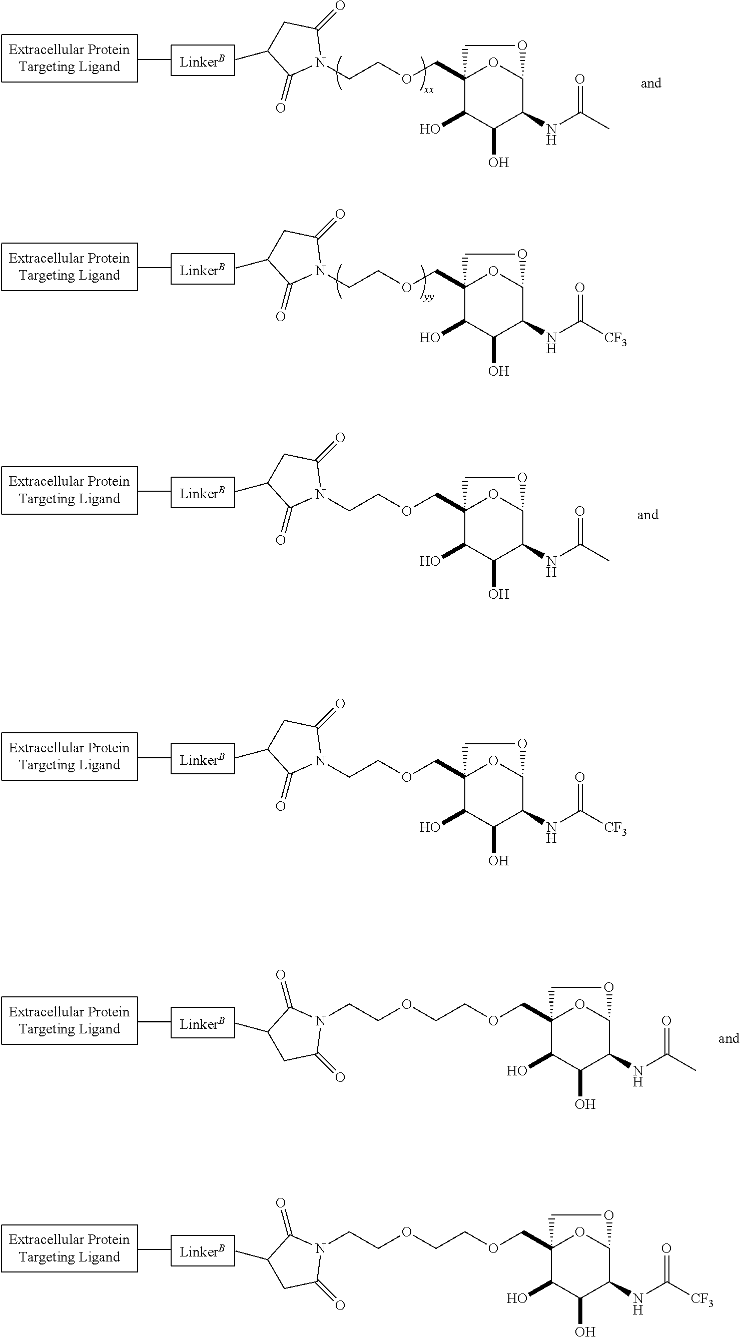

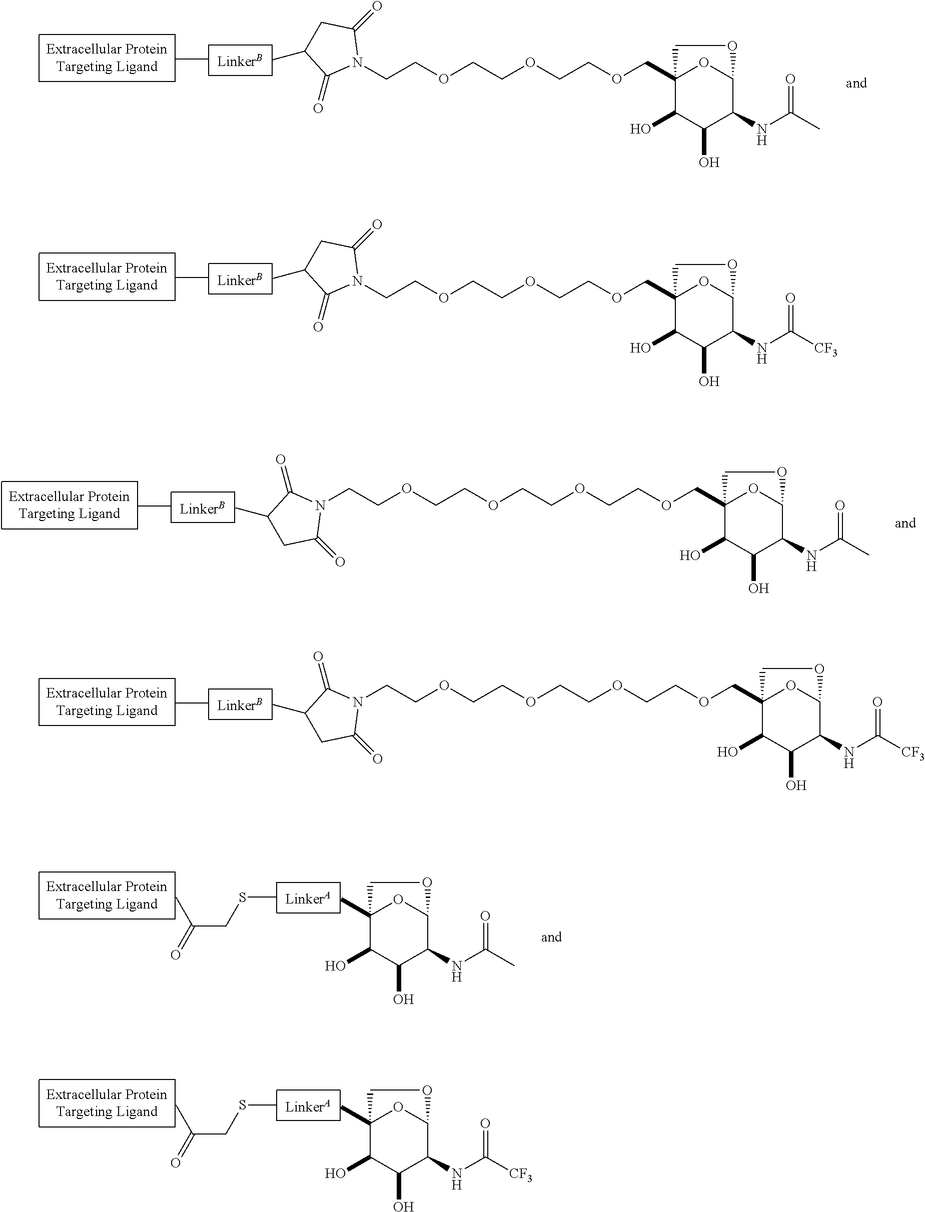

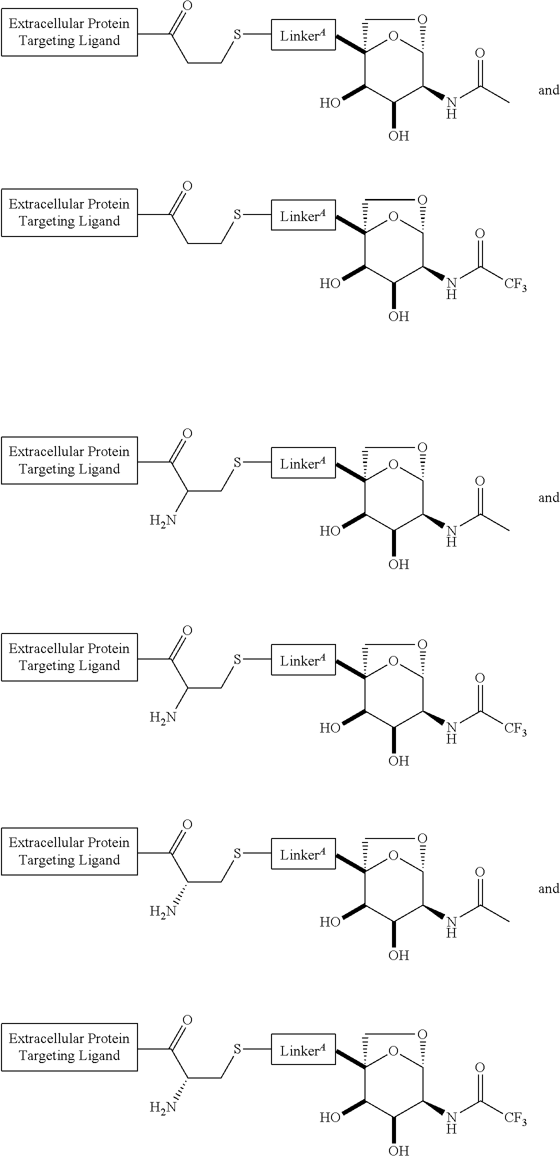

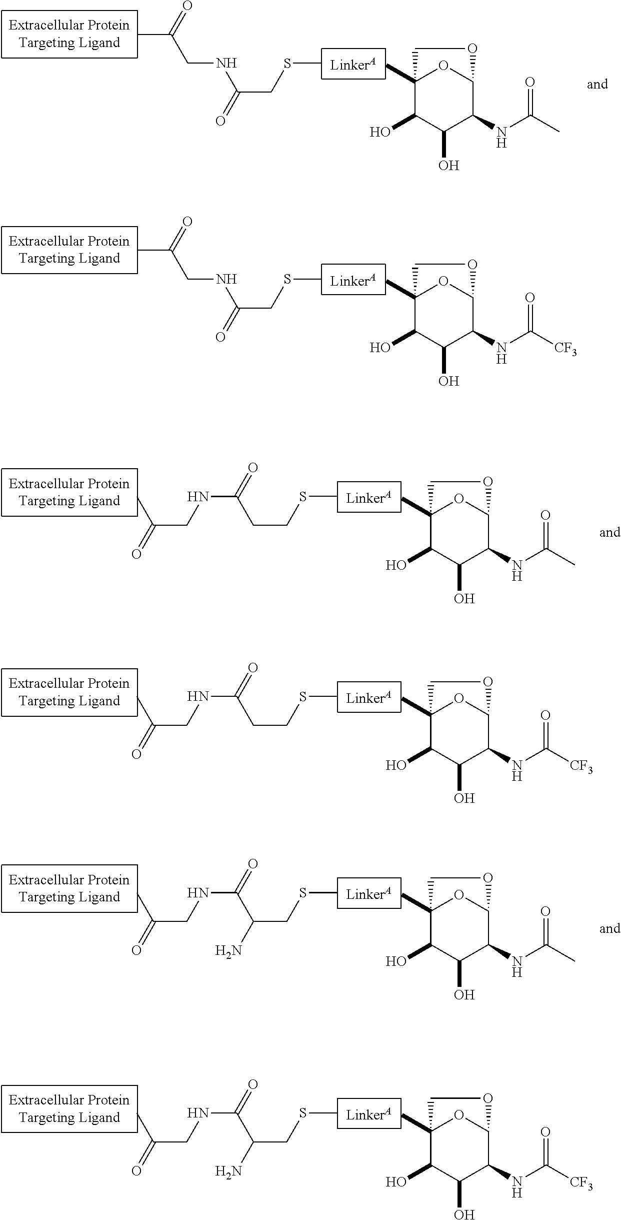

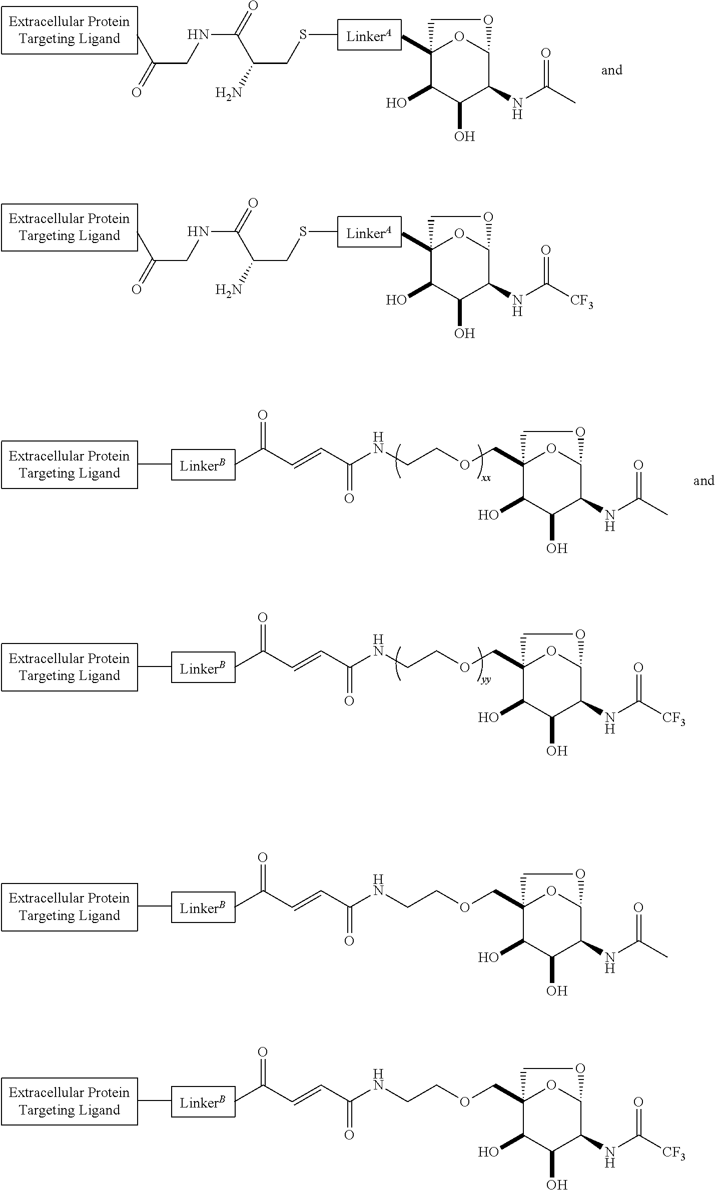

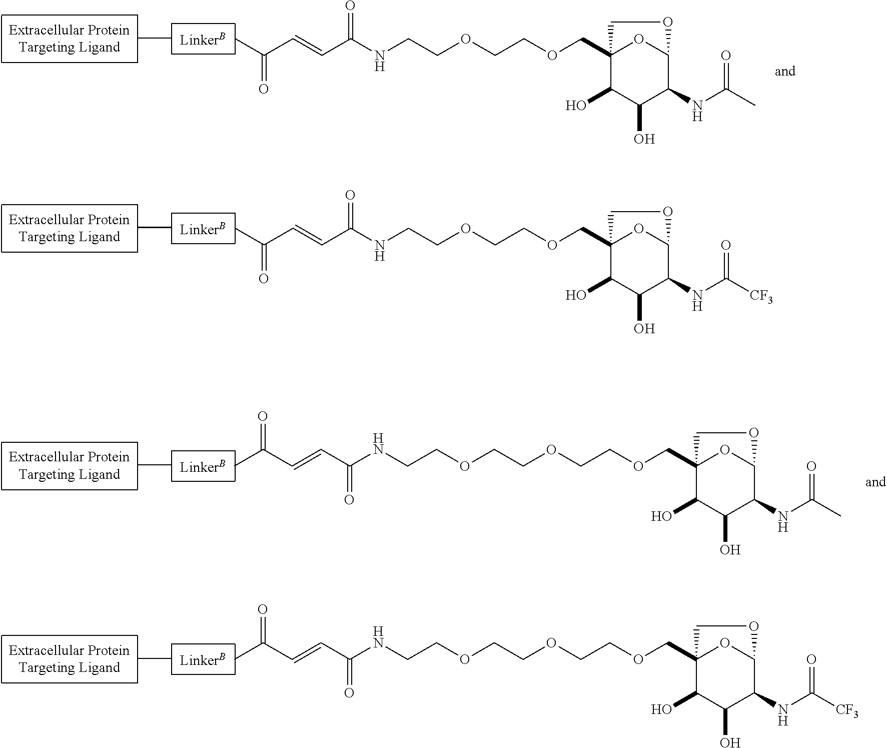

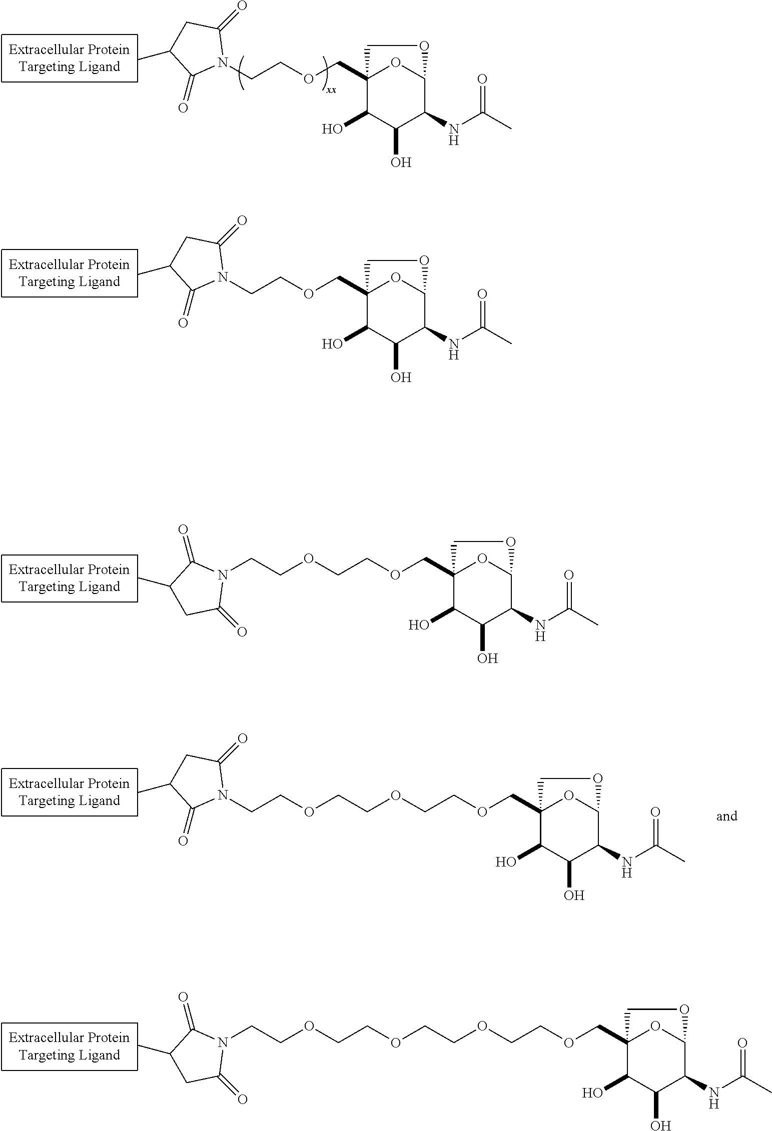

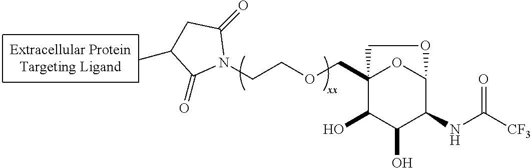

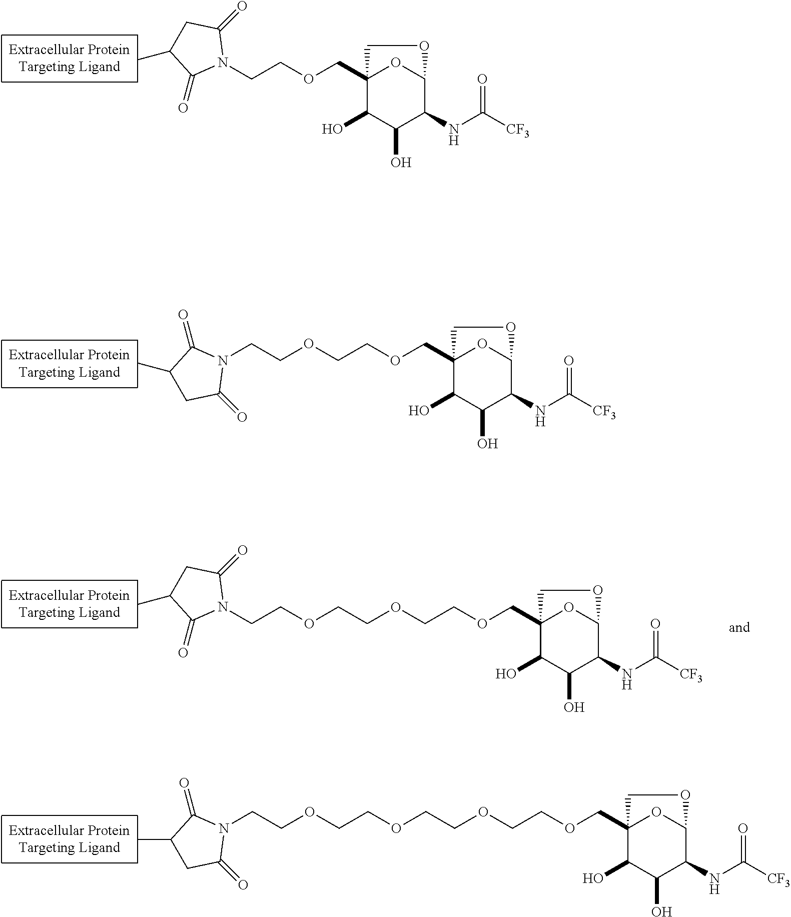

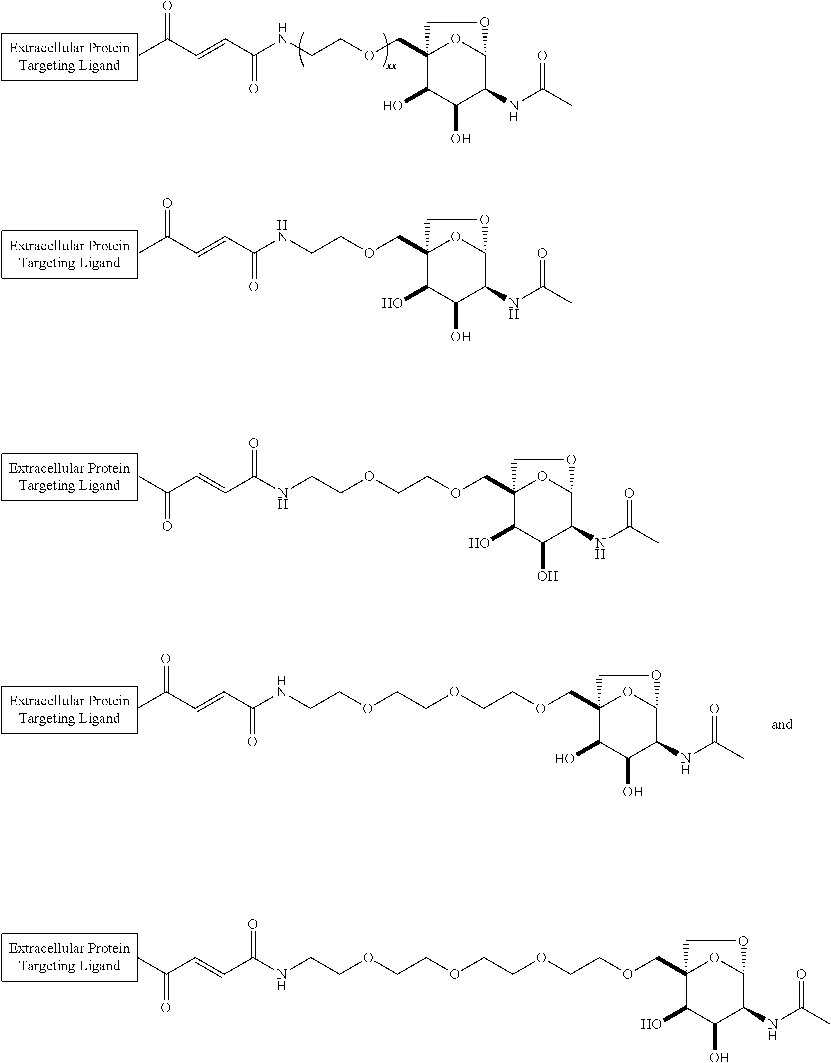

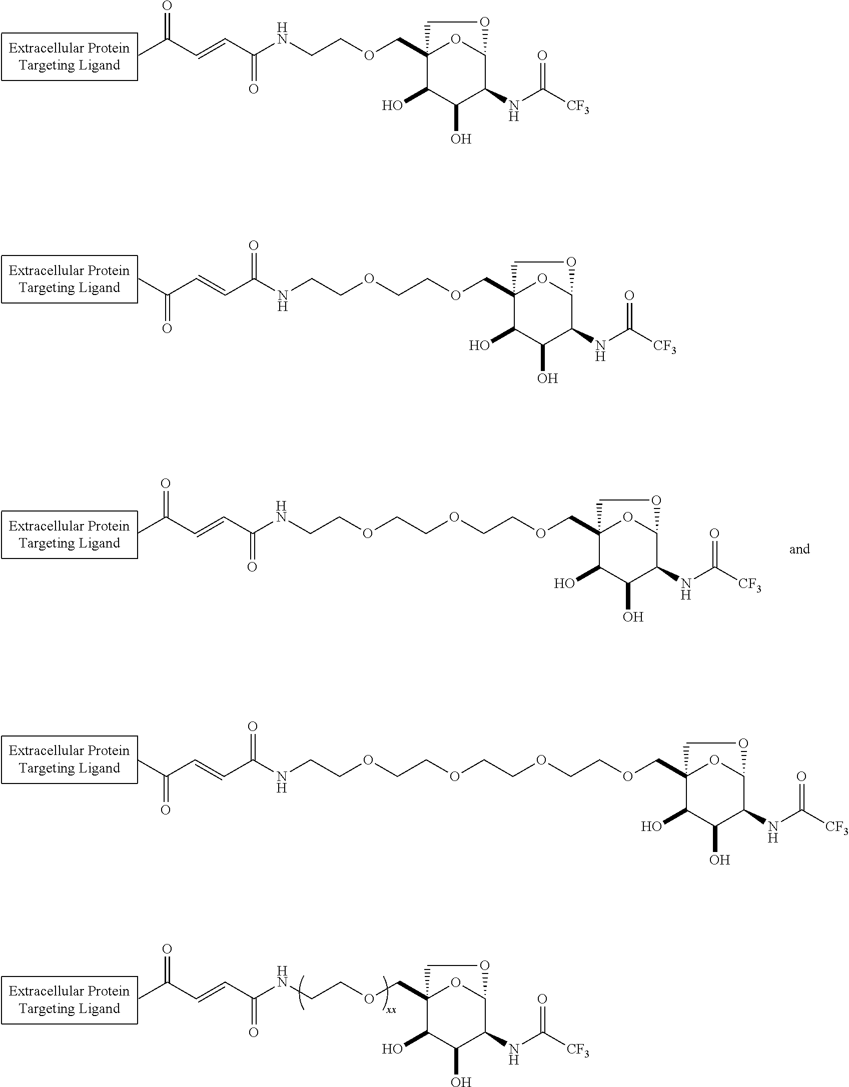

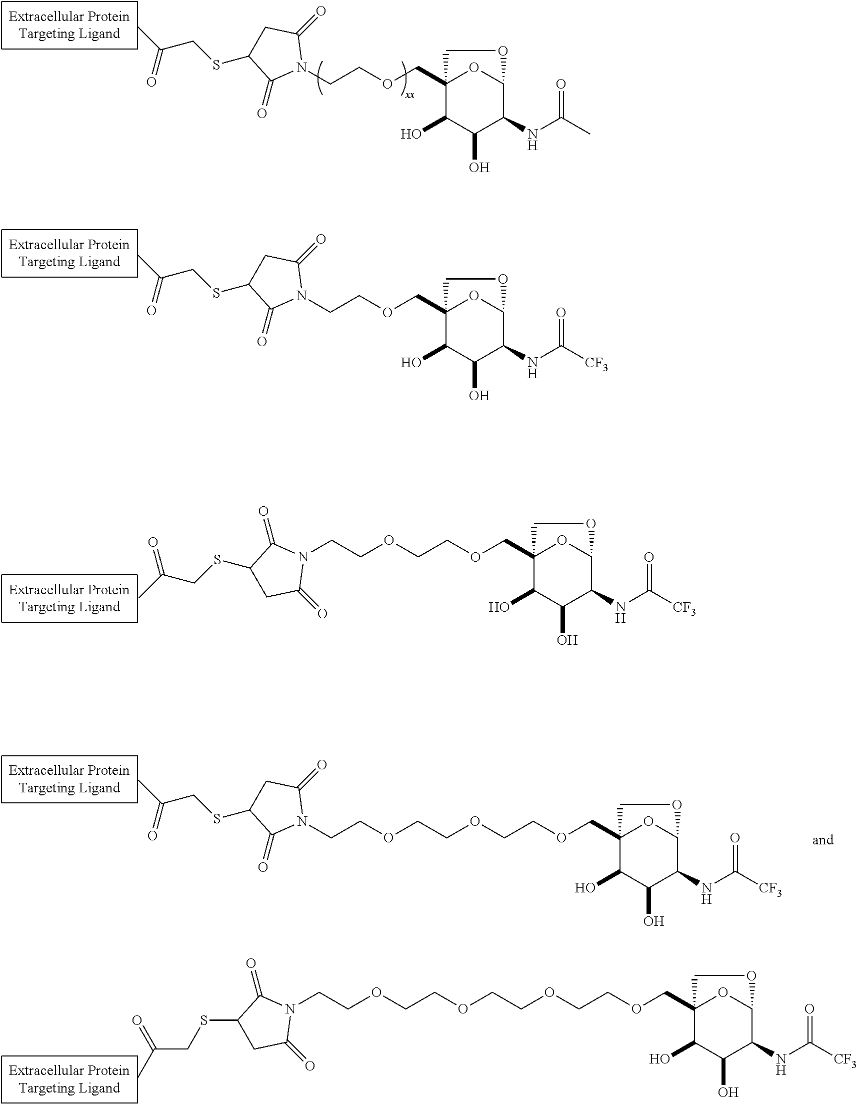

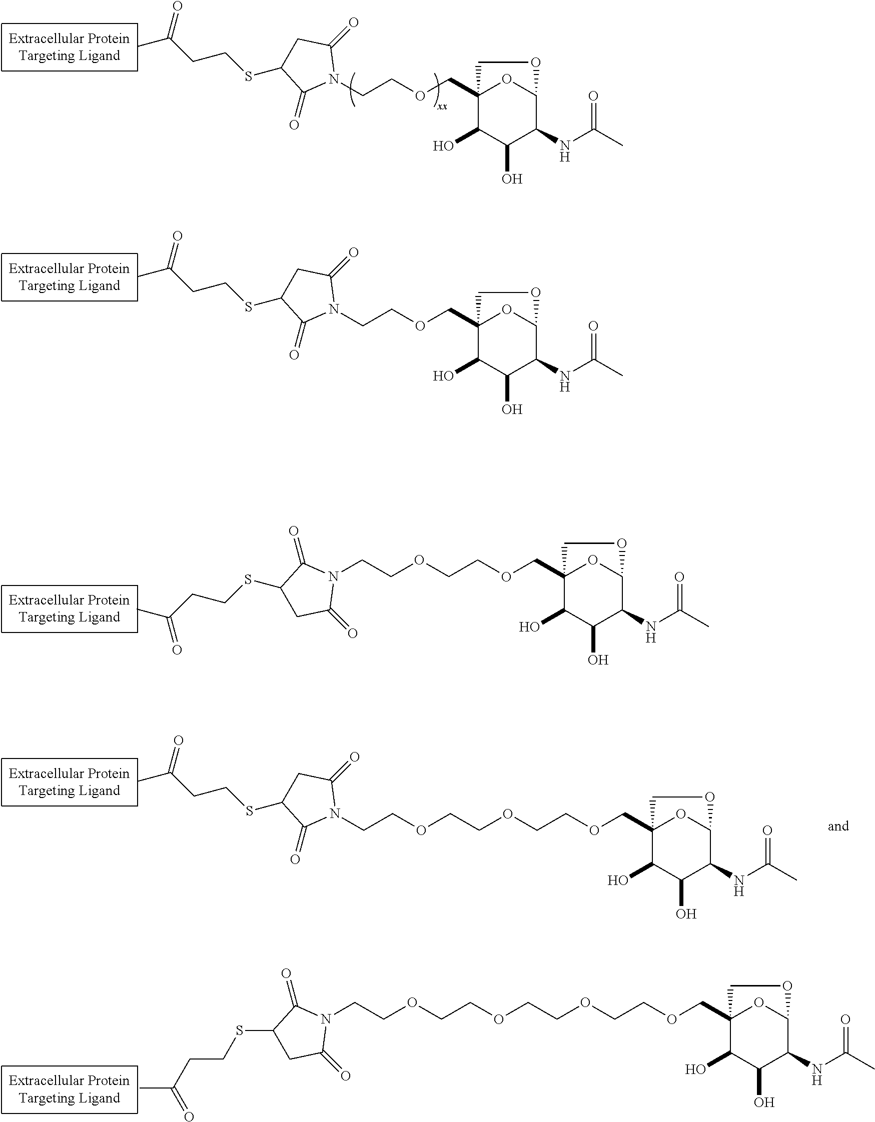

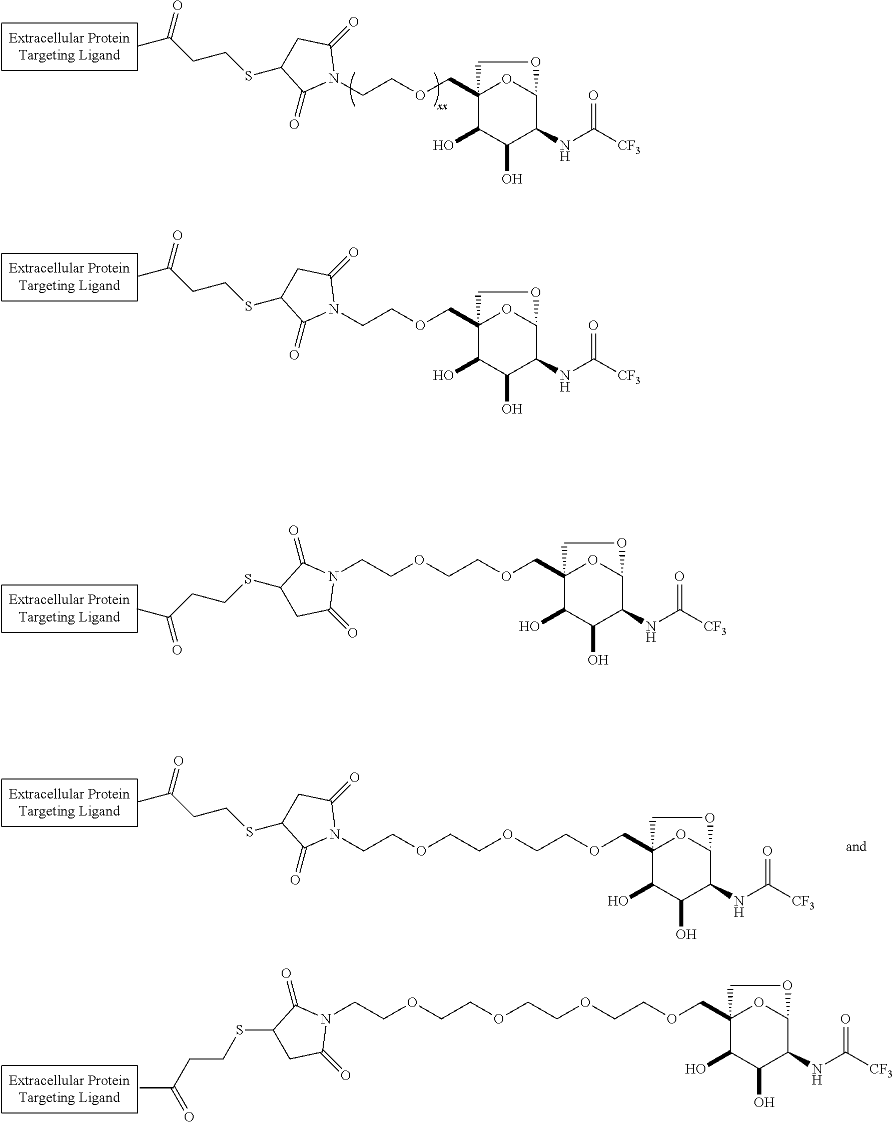

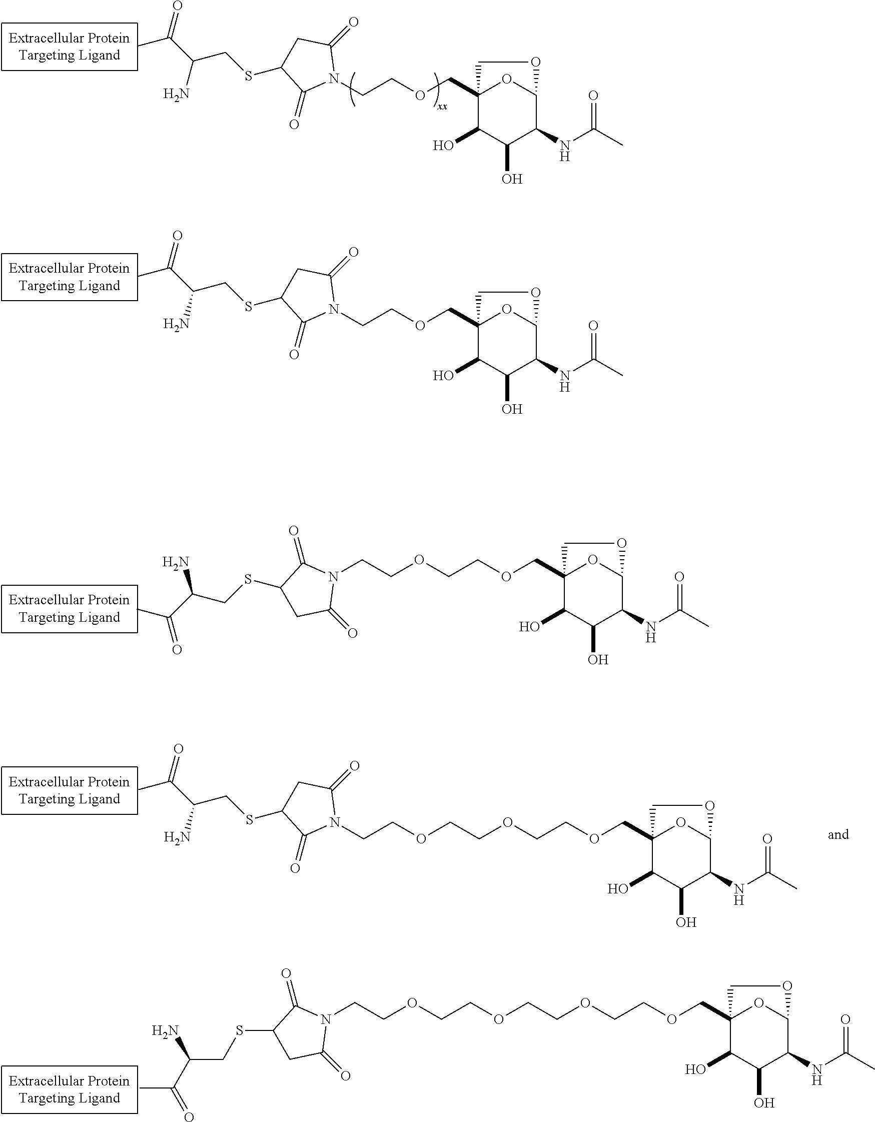

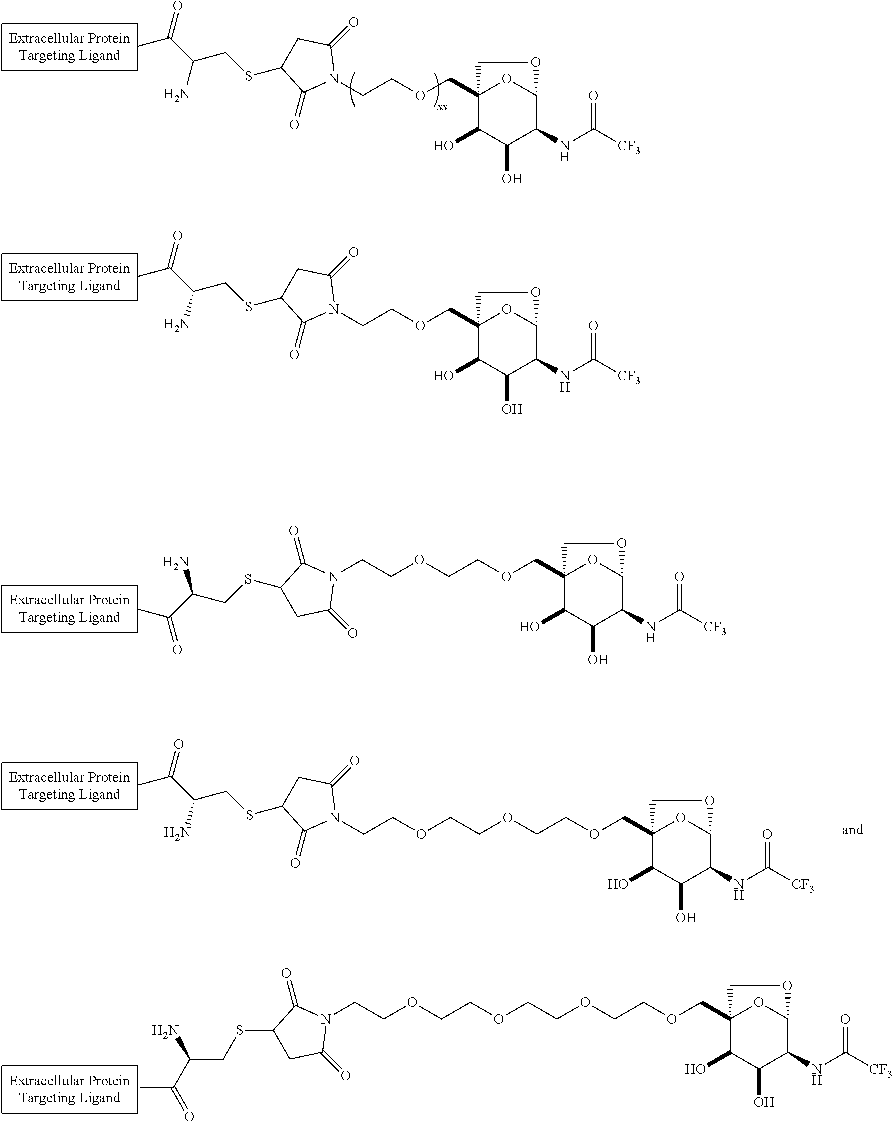

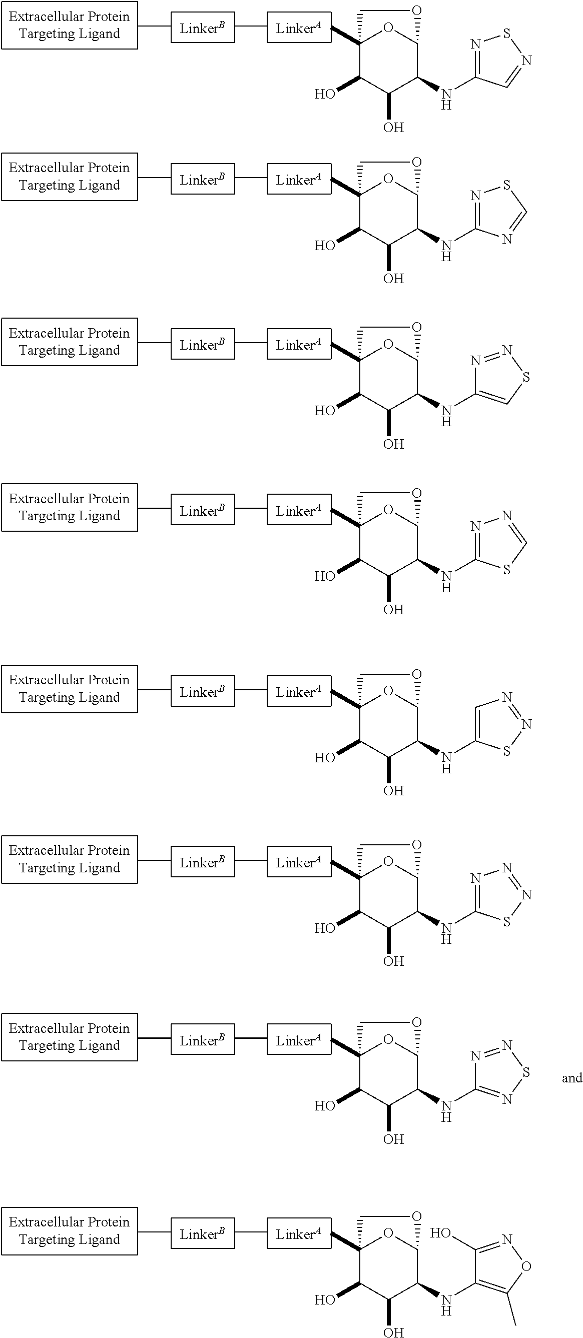

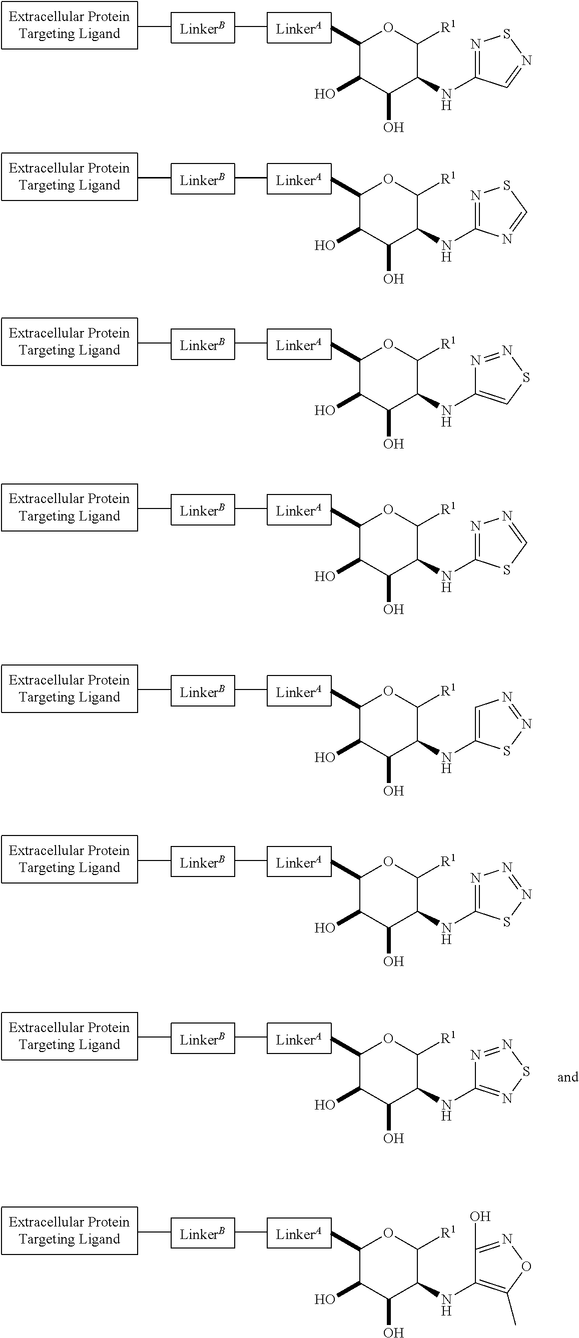

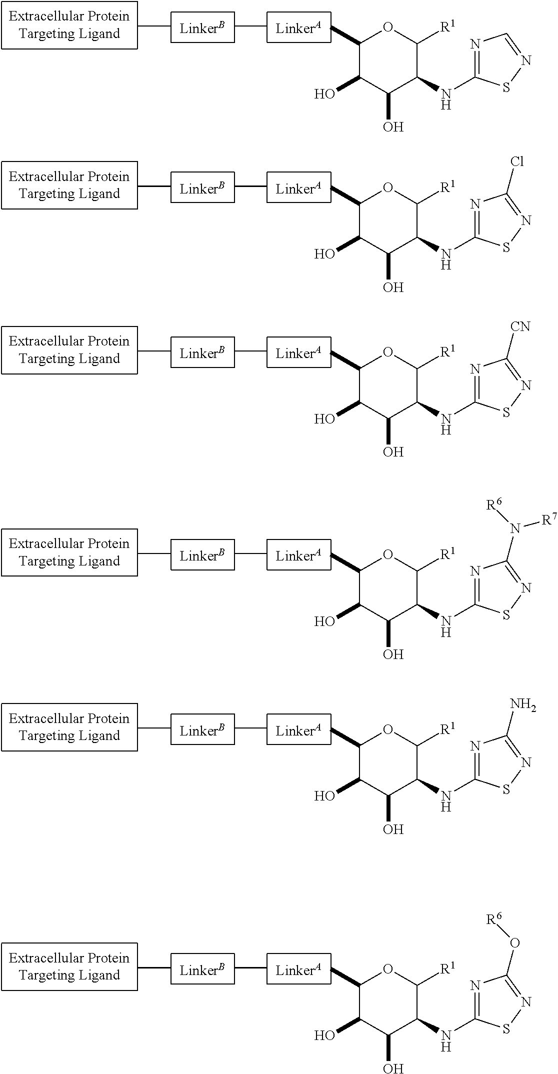

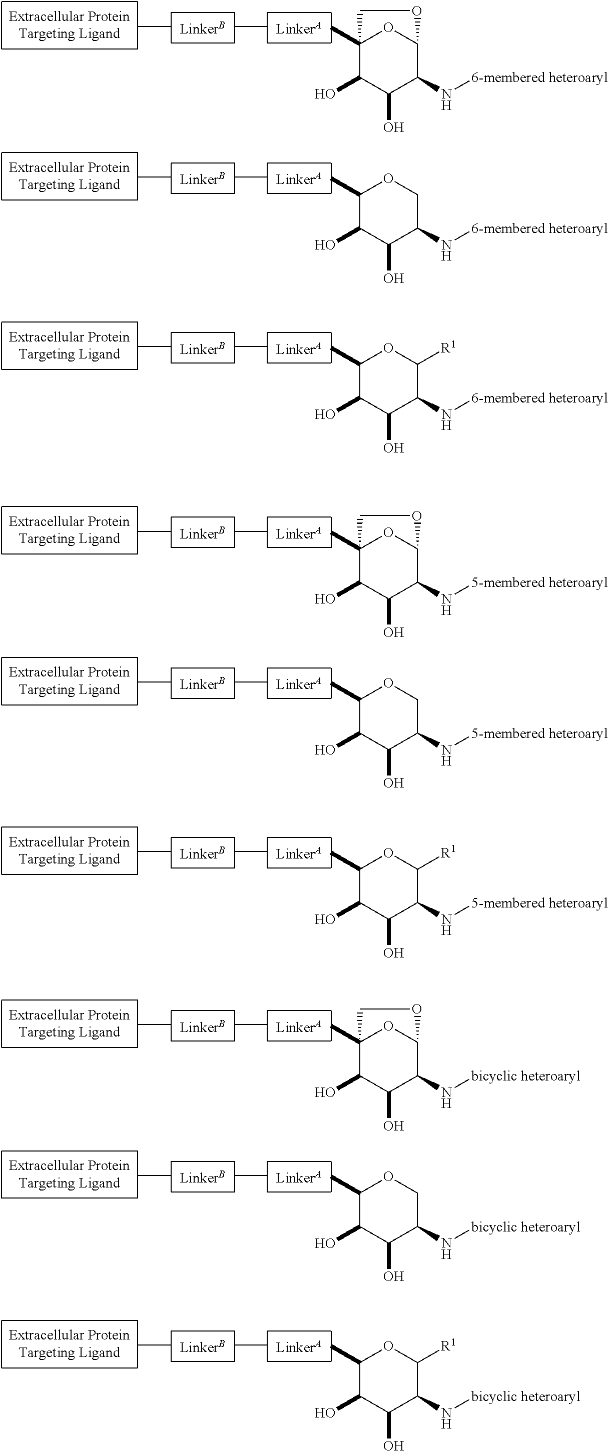

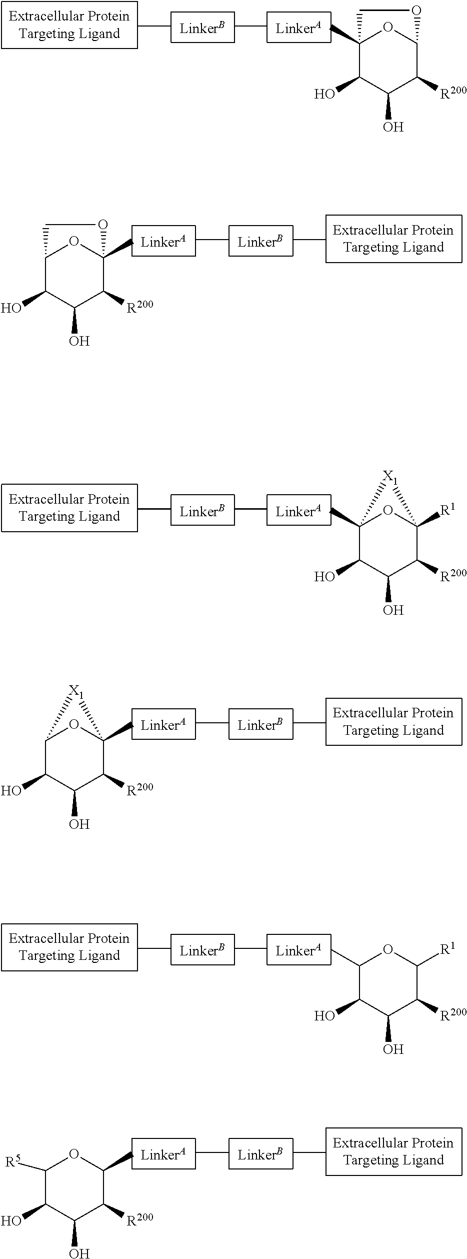

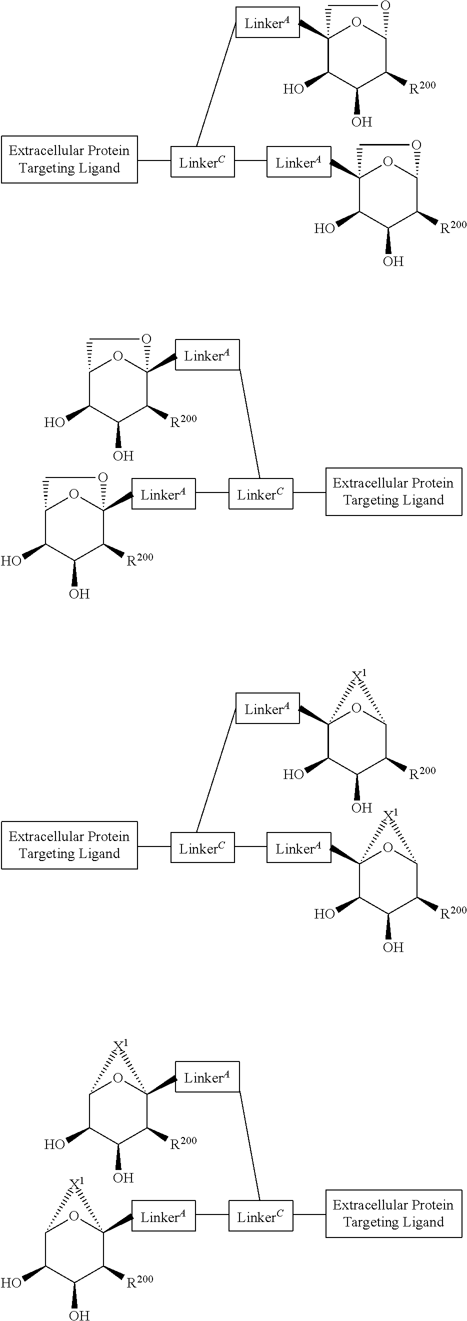

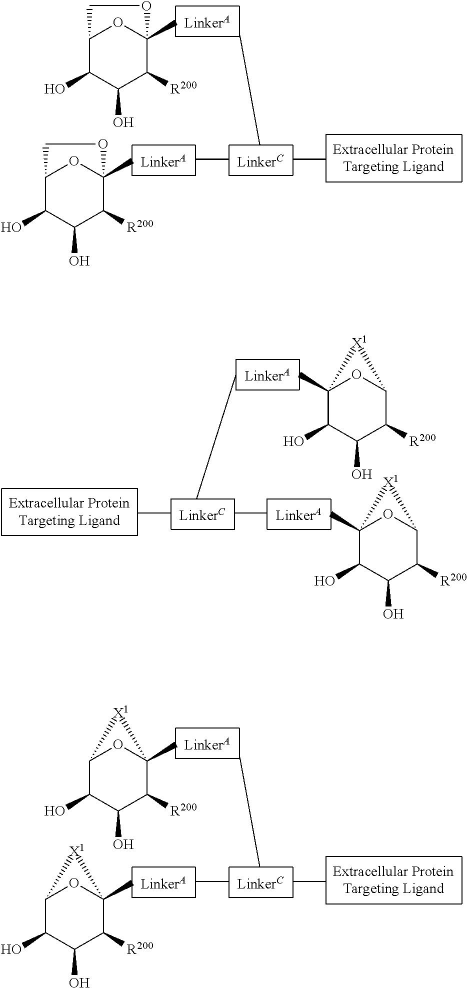

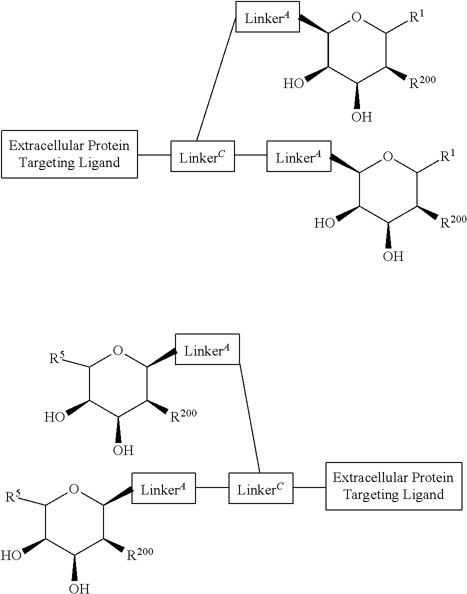

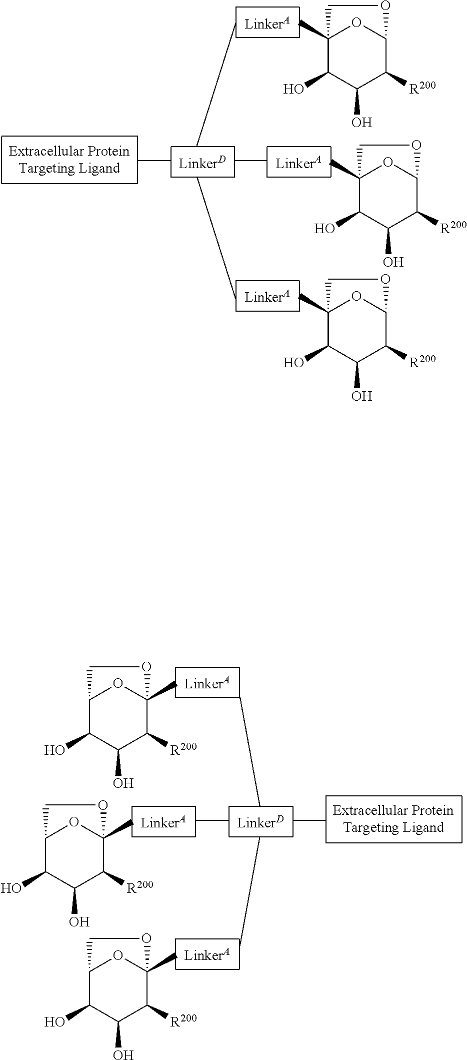

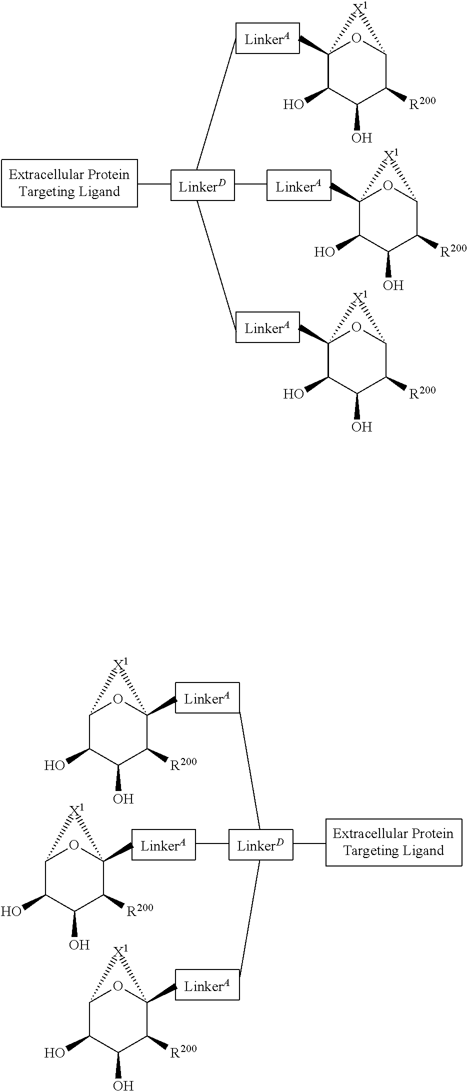

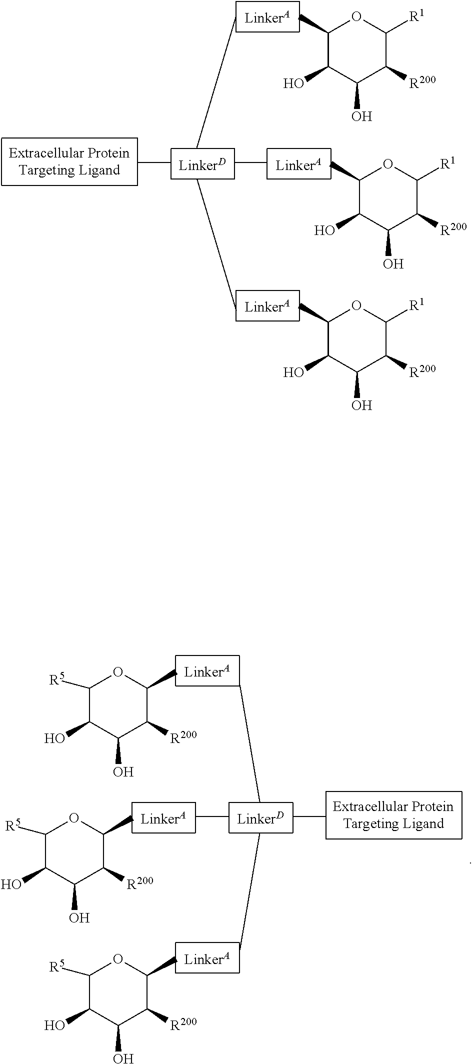

In one embodiment, the compound of Formula II is an Extracellular Protein degrading compound in which the ASGPR ligand is a ligand as described herein

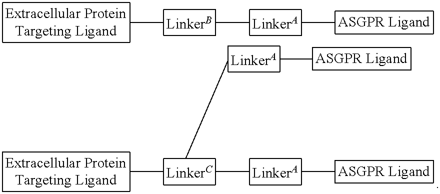

In one embodiment, in the compound of Formula II, the ASGPR ligand is linked at either the C1 or C5 (R¹ or R⁵) position to form a degrading compound. In one embodiment, in the compound of Formula II, the ASGPR ligand is linked at C6. In various embodiments, when the ASGPR ligand is

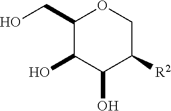

then non-limiting examples of ASGPR binding compounds of Formula II include:

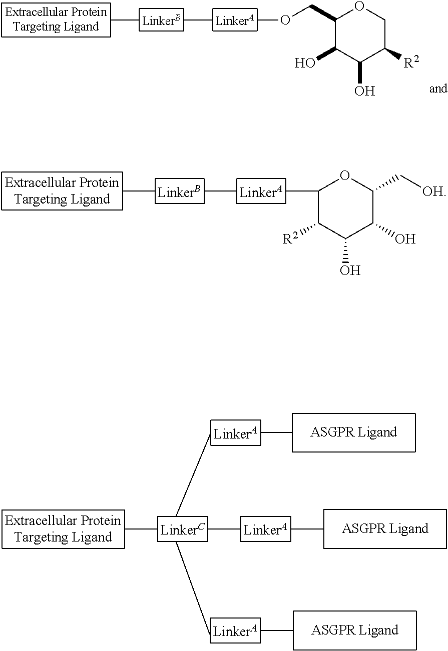

or the bi- or tri-substituted versions thereof or pharmaceutically acceptable salts thereof, where the bi- or tri-substitution refers to the number additional galactose derivatives attached to a linker moiety. In certain embodiments the compound of Formula II is selected from:

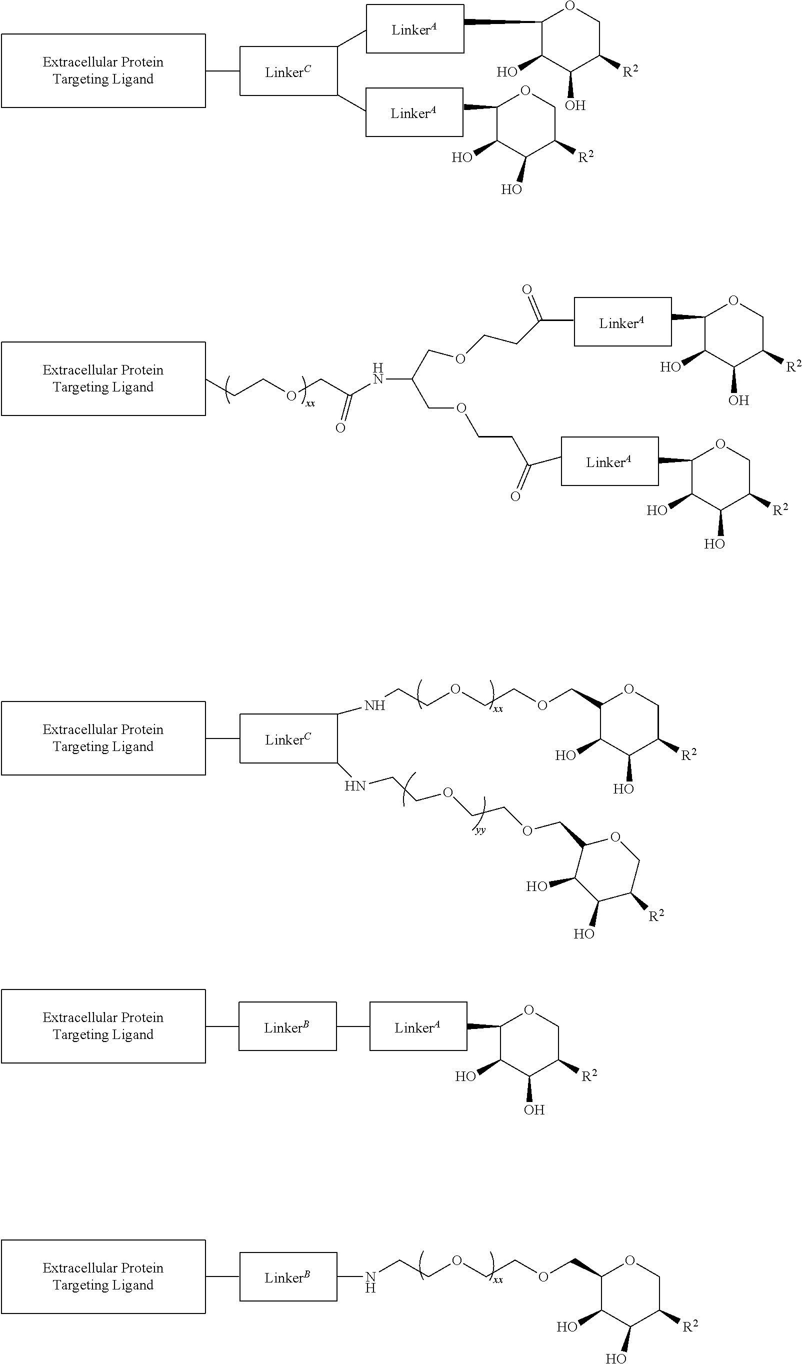

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

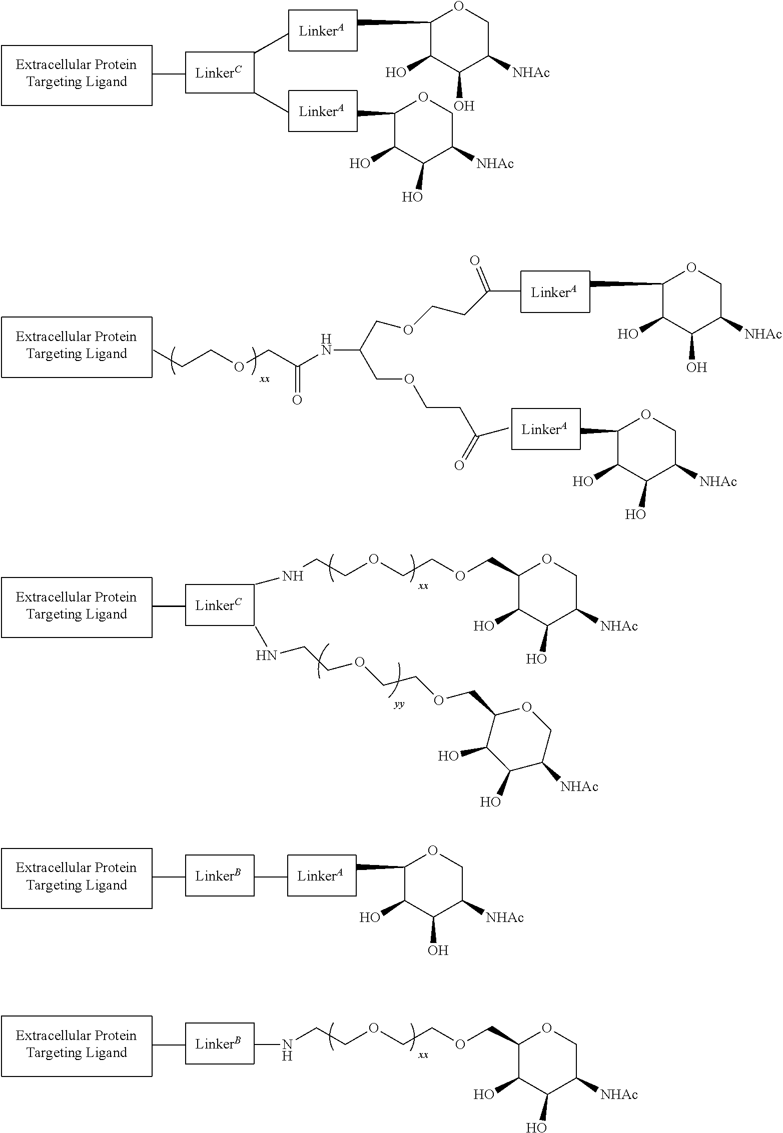

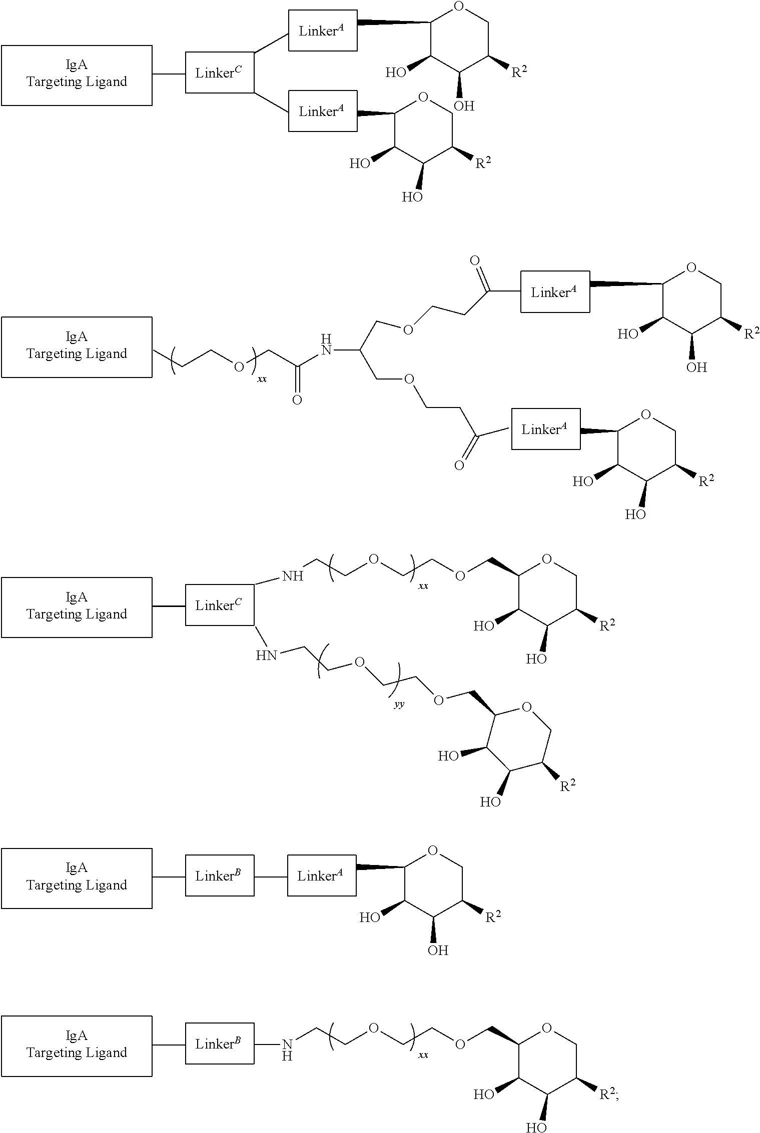

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

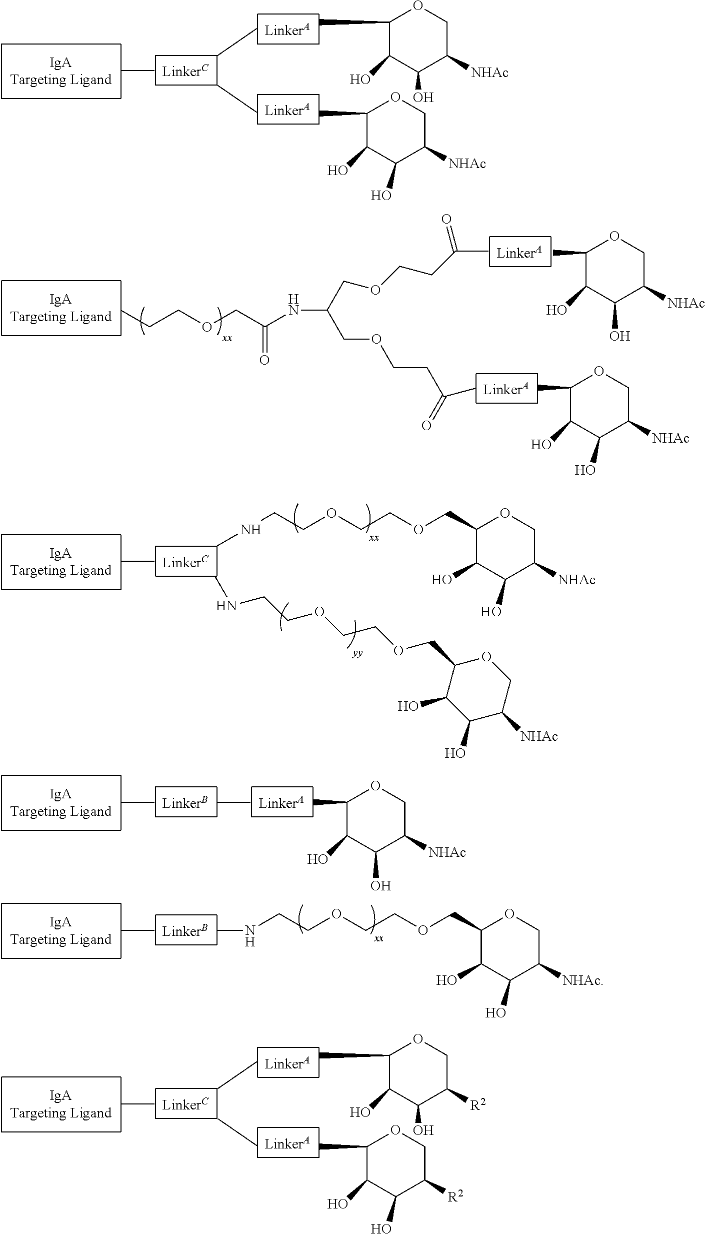

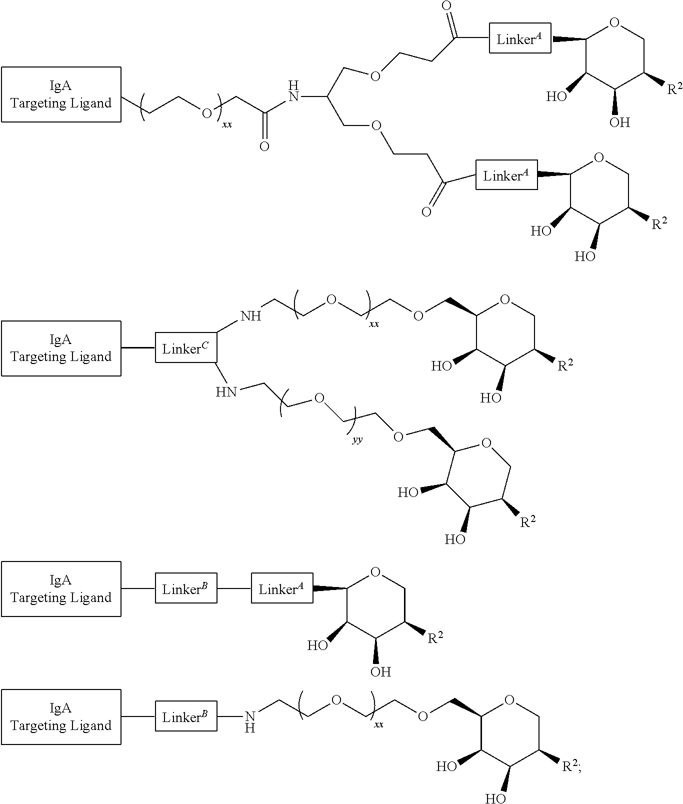

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

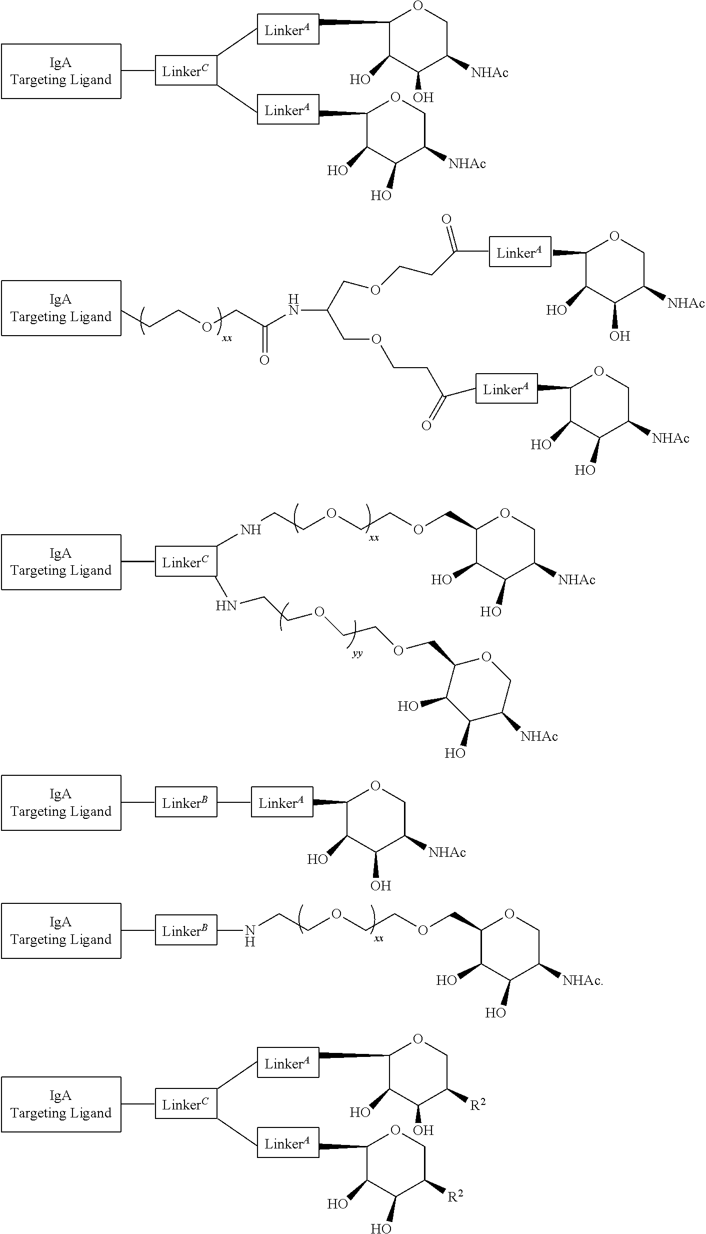

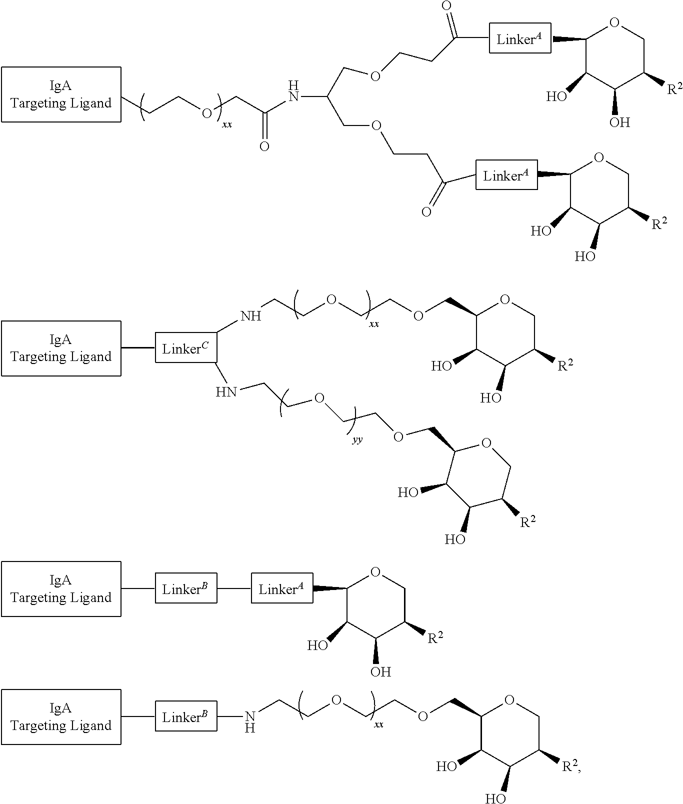

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

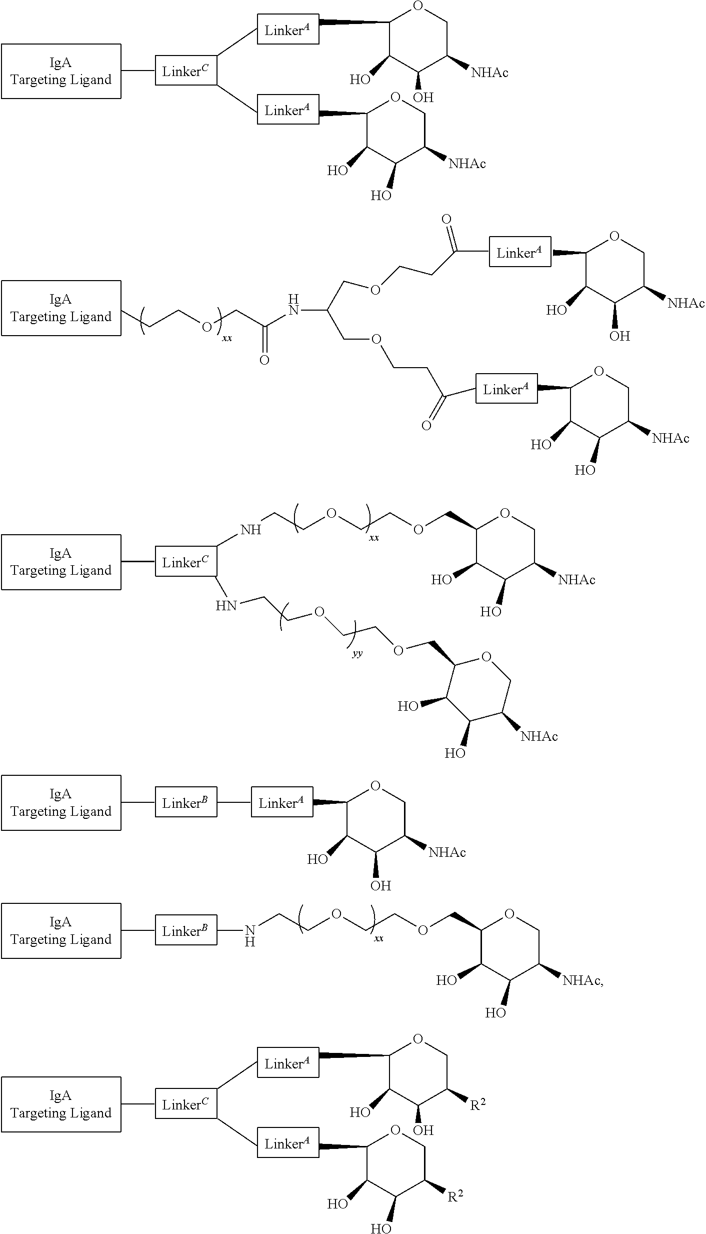

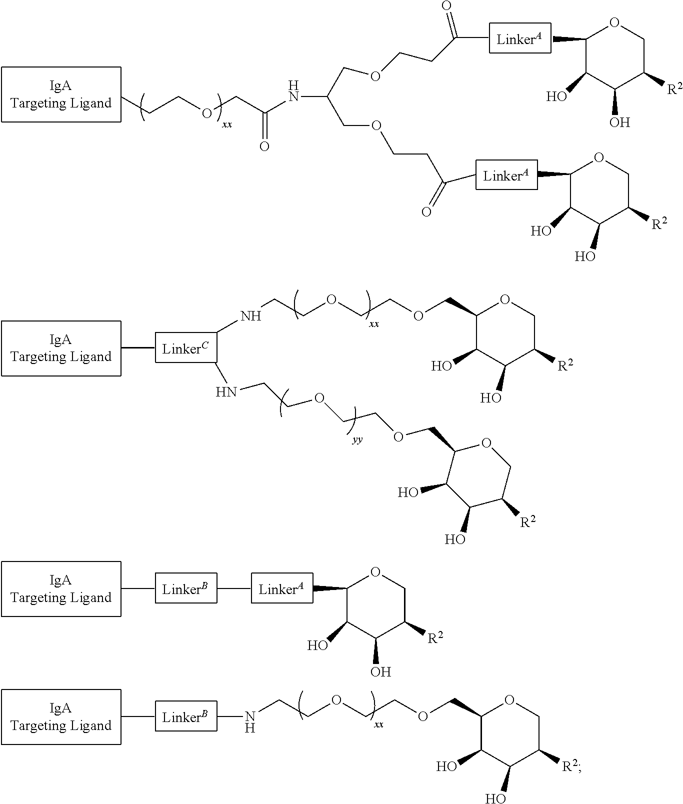

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

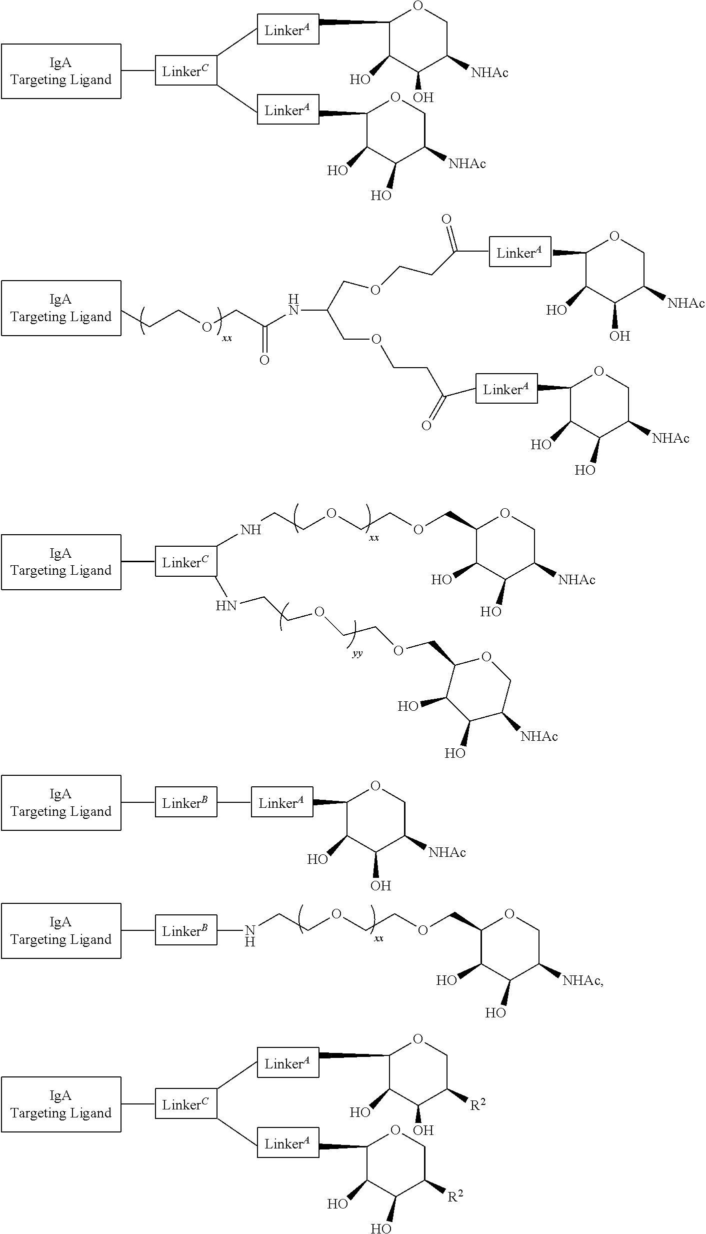

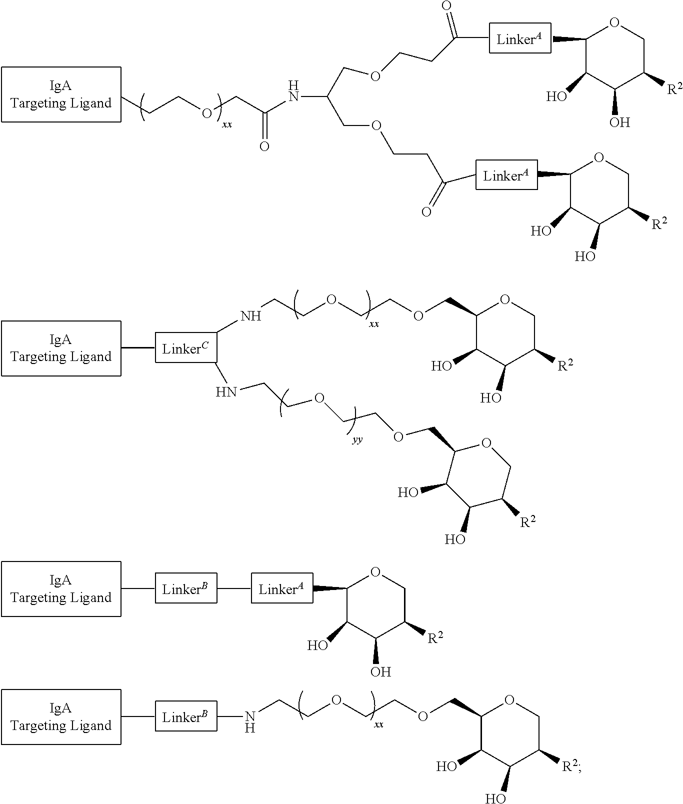

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

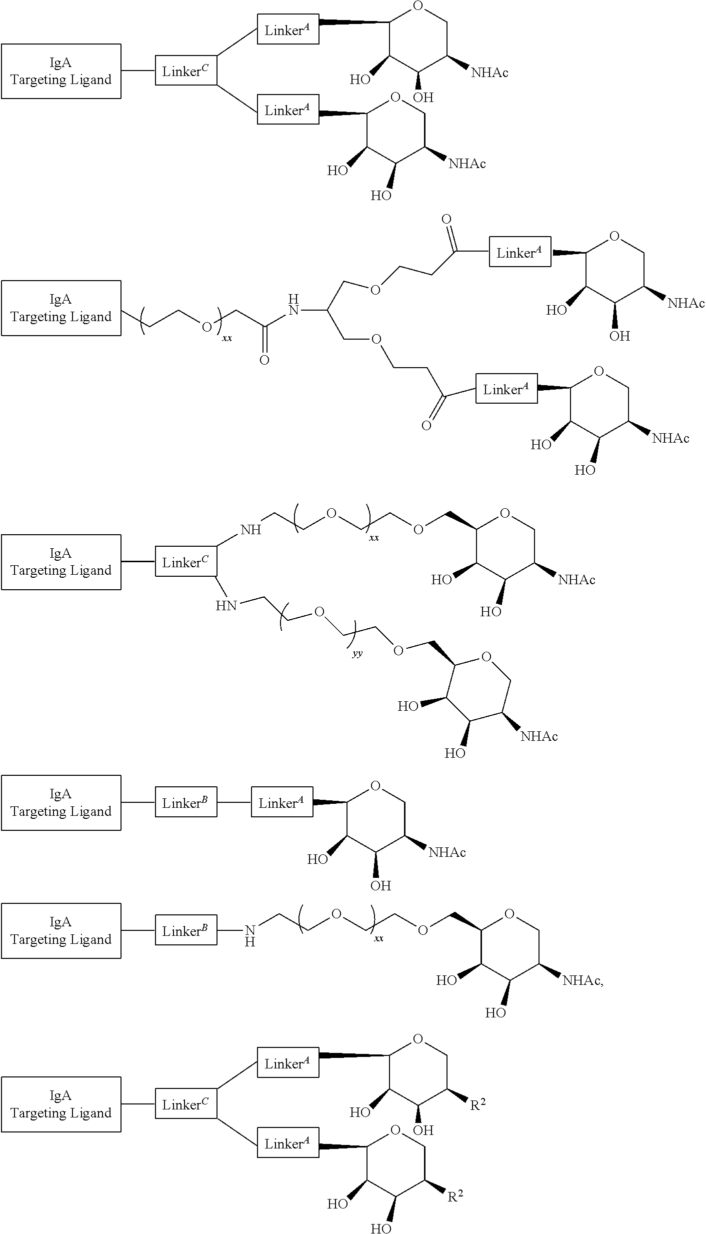

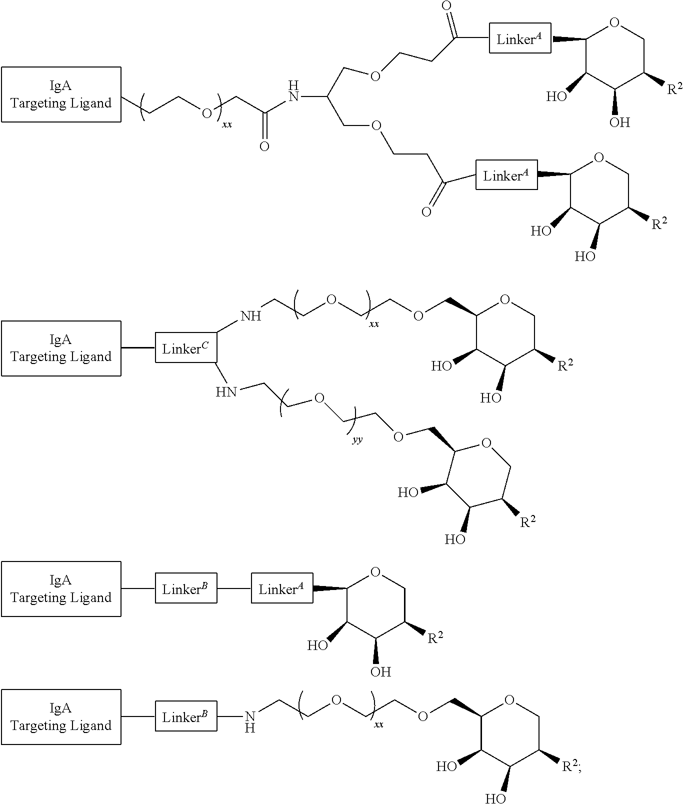

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

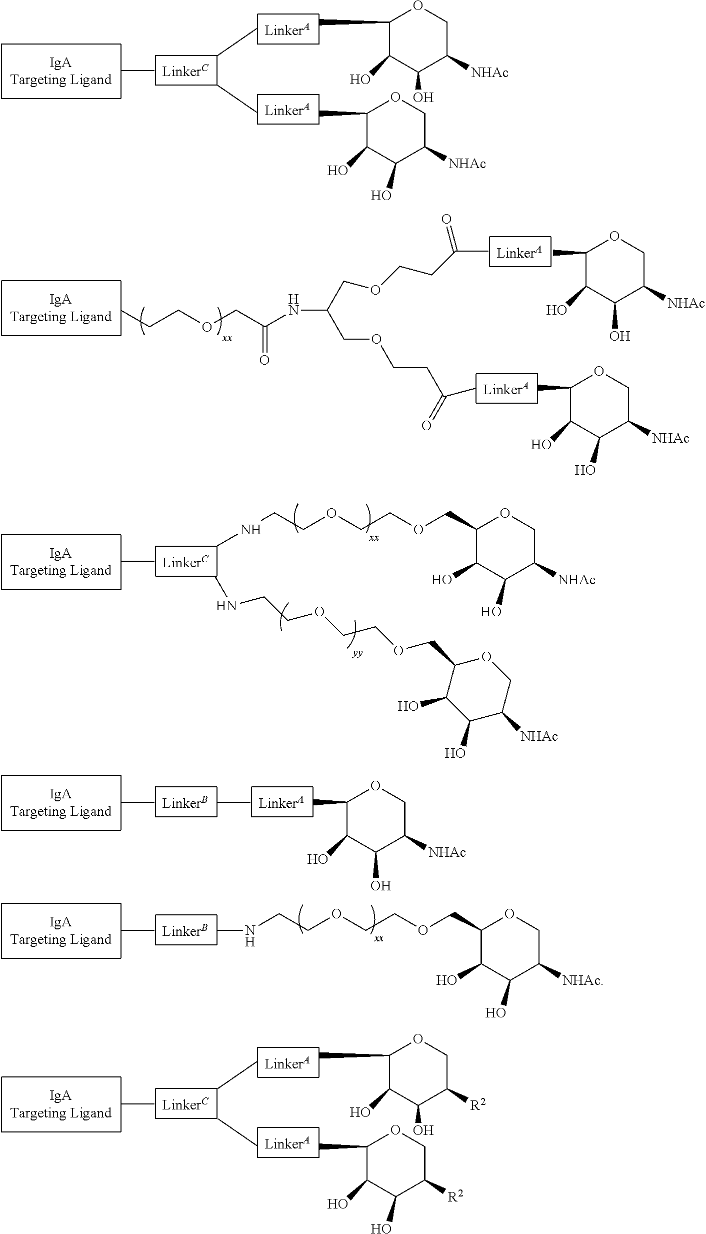

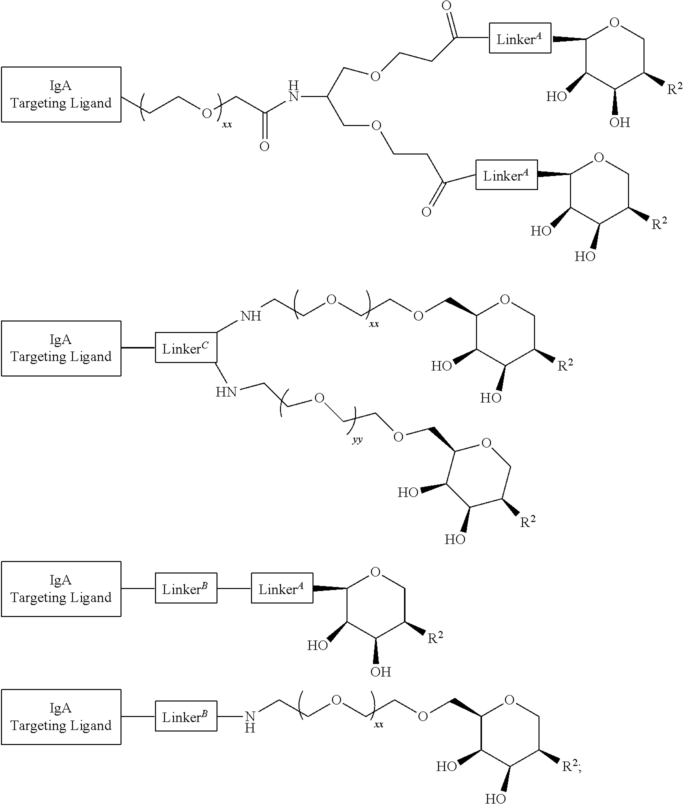

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

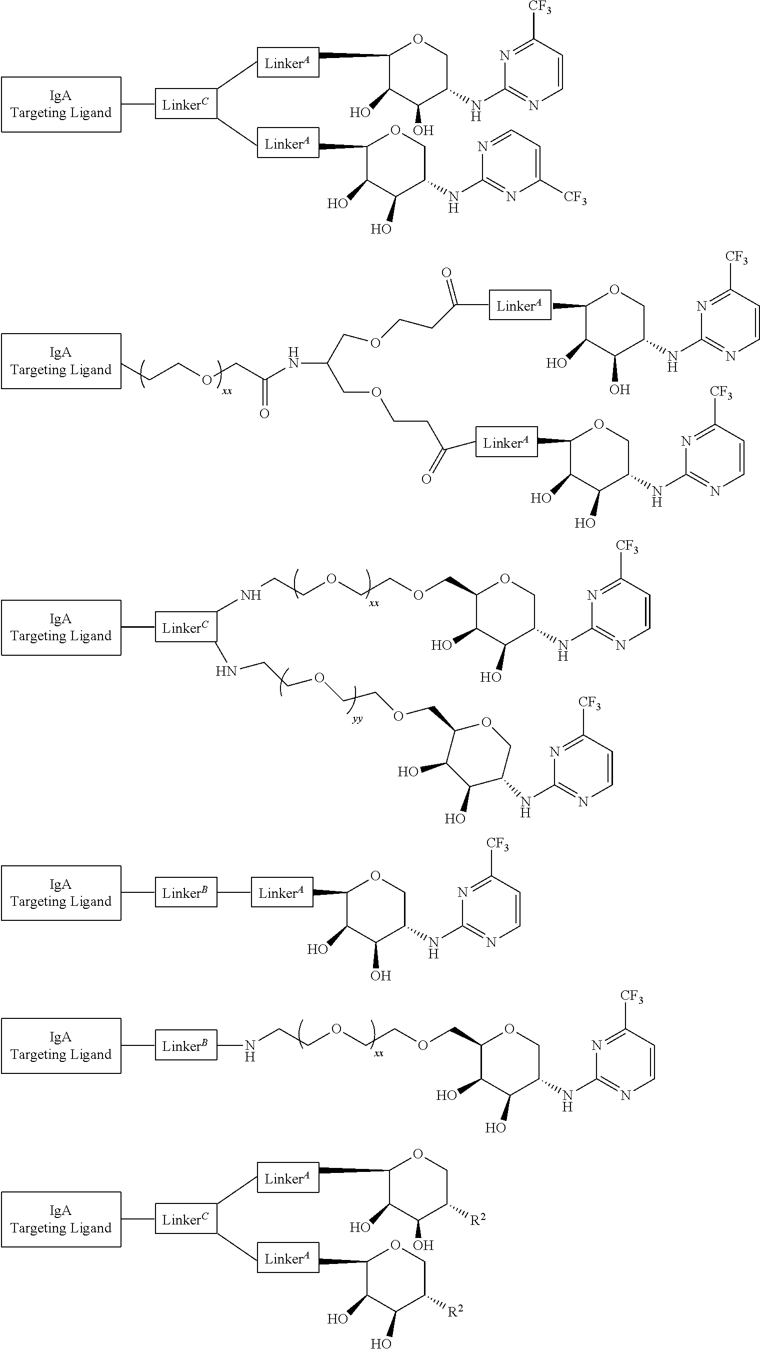

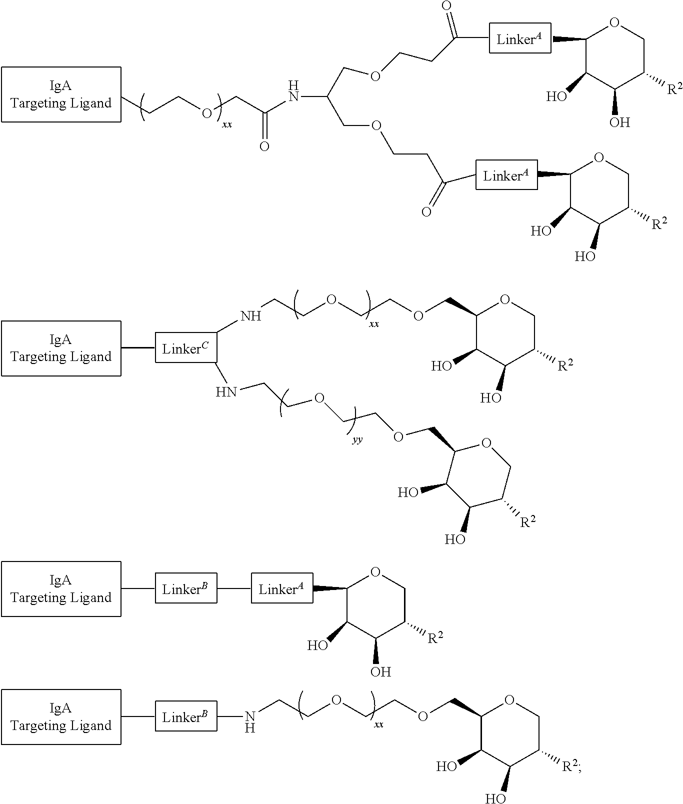

wherein in certain embodiments R² is selected from —NRbCOR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

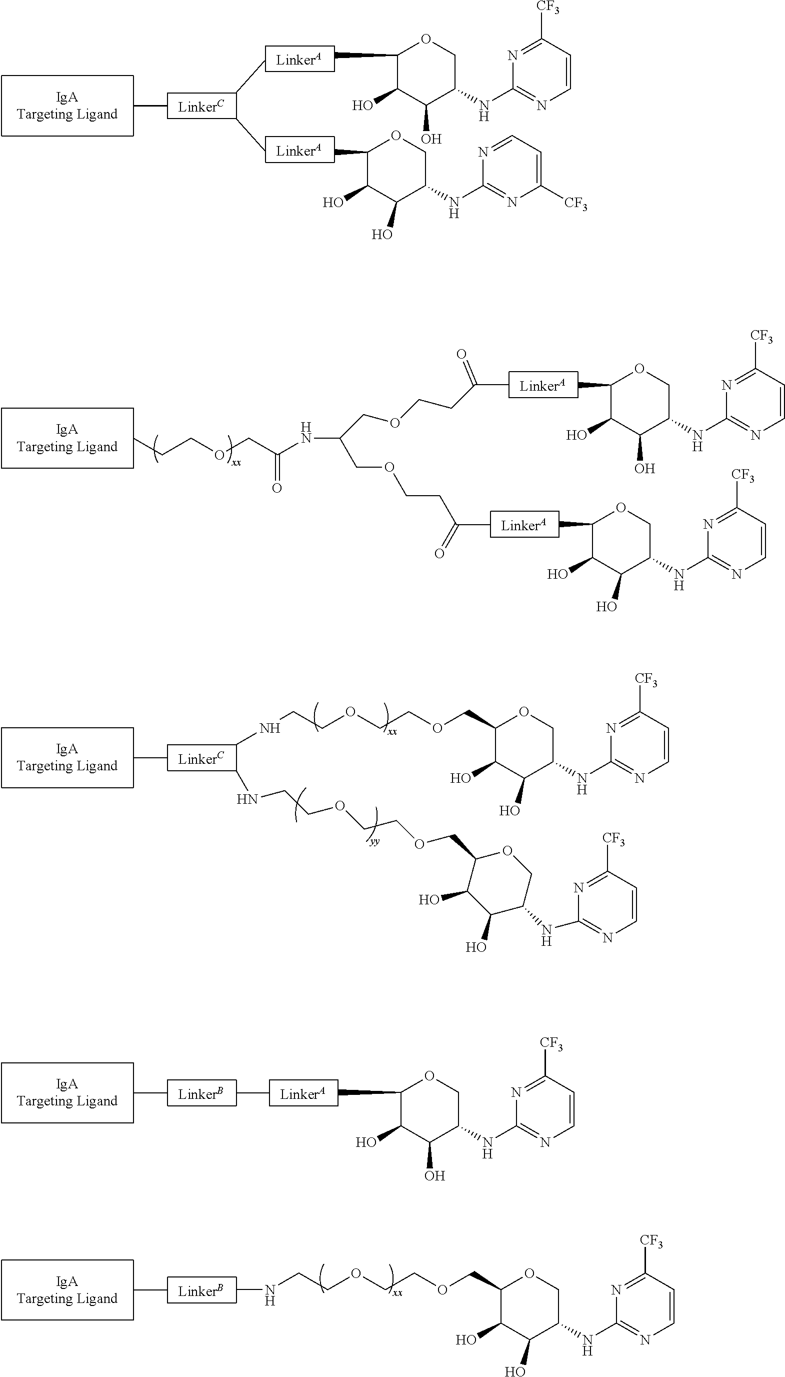

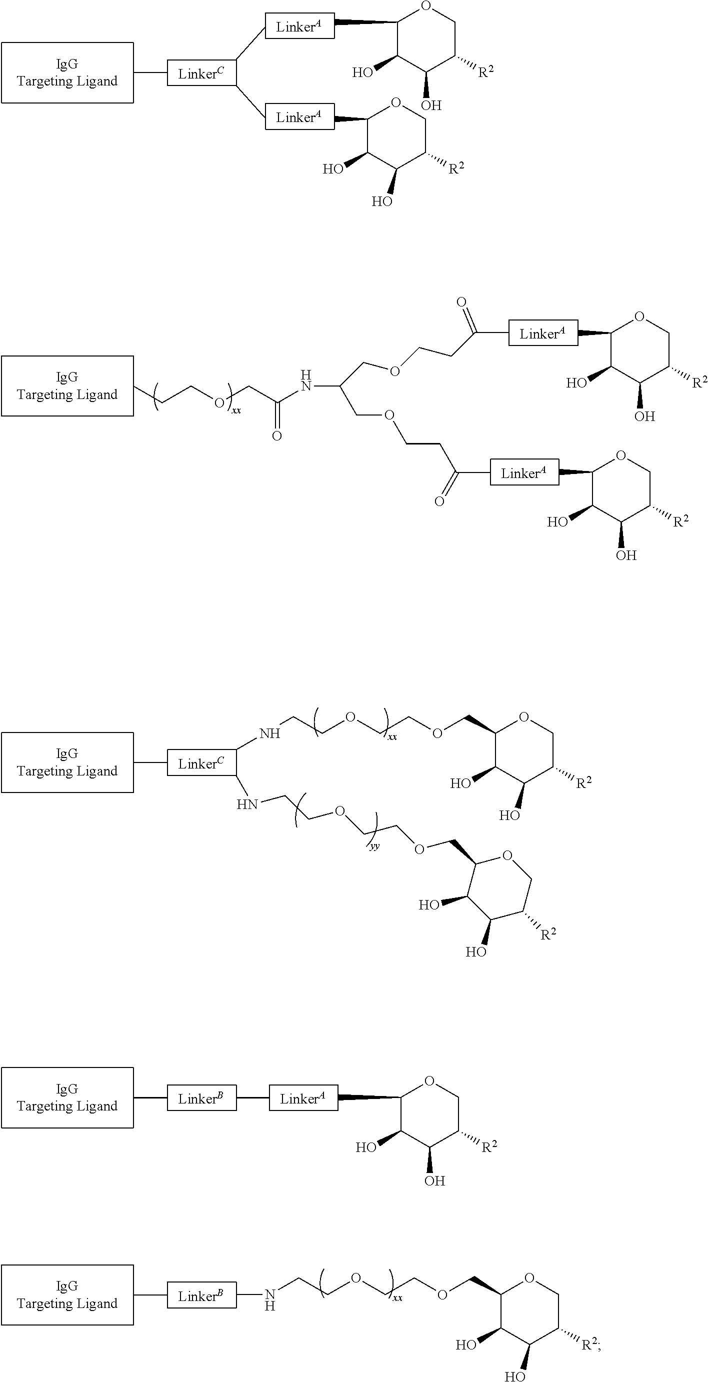

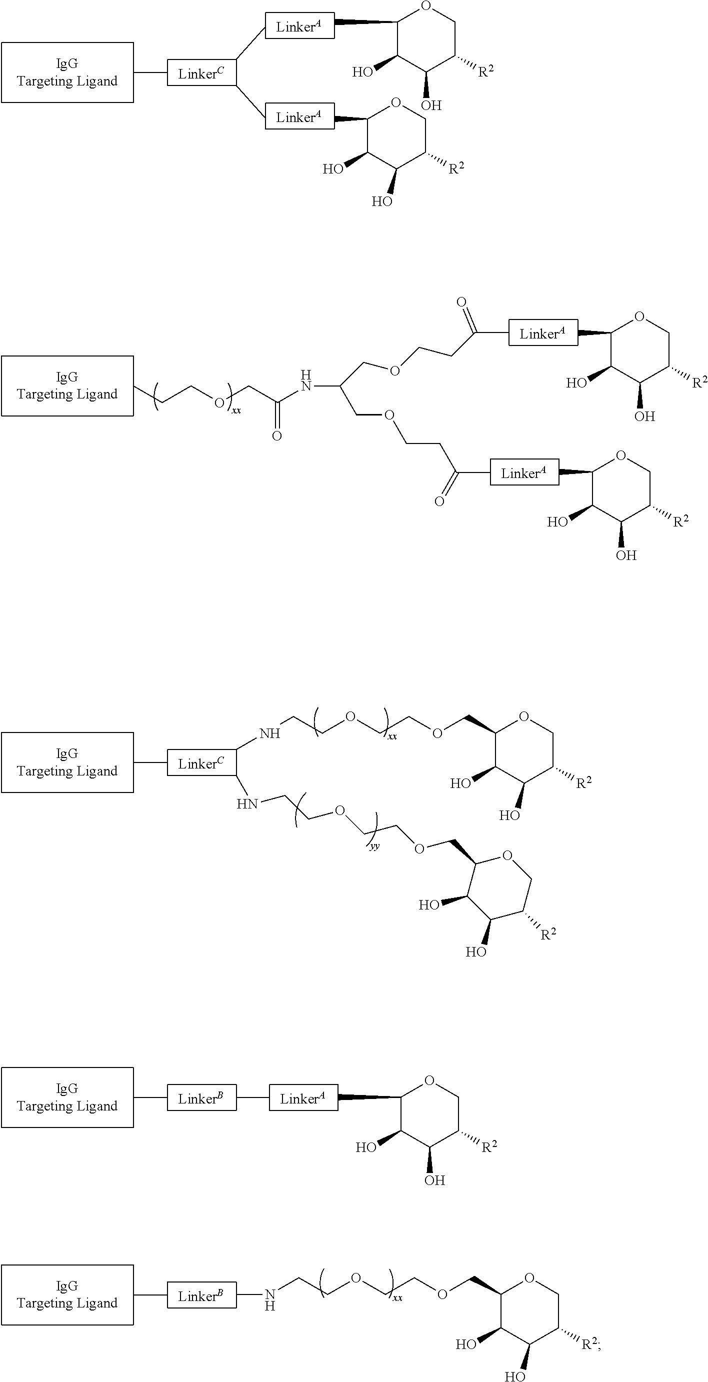

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

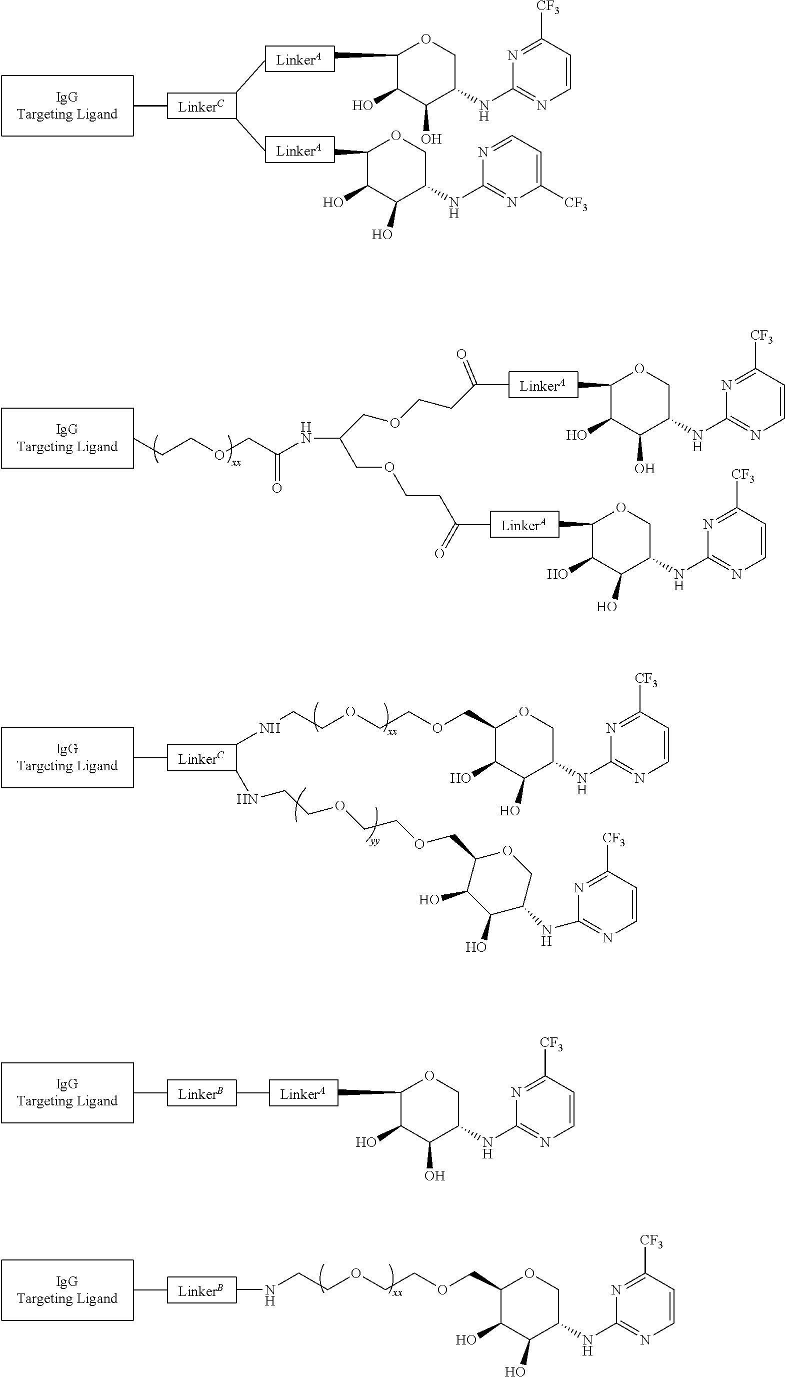

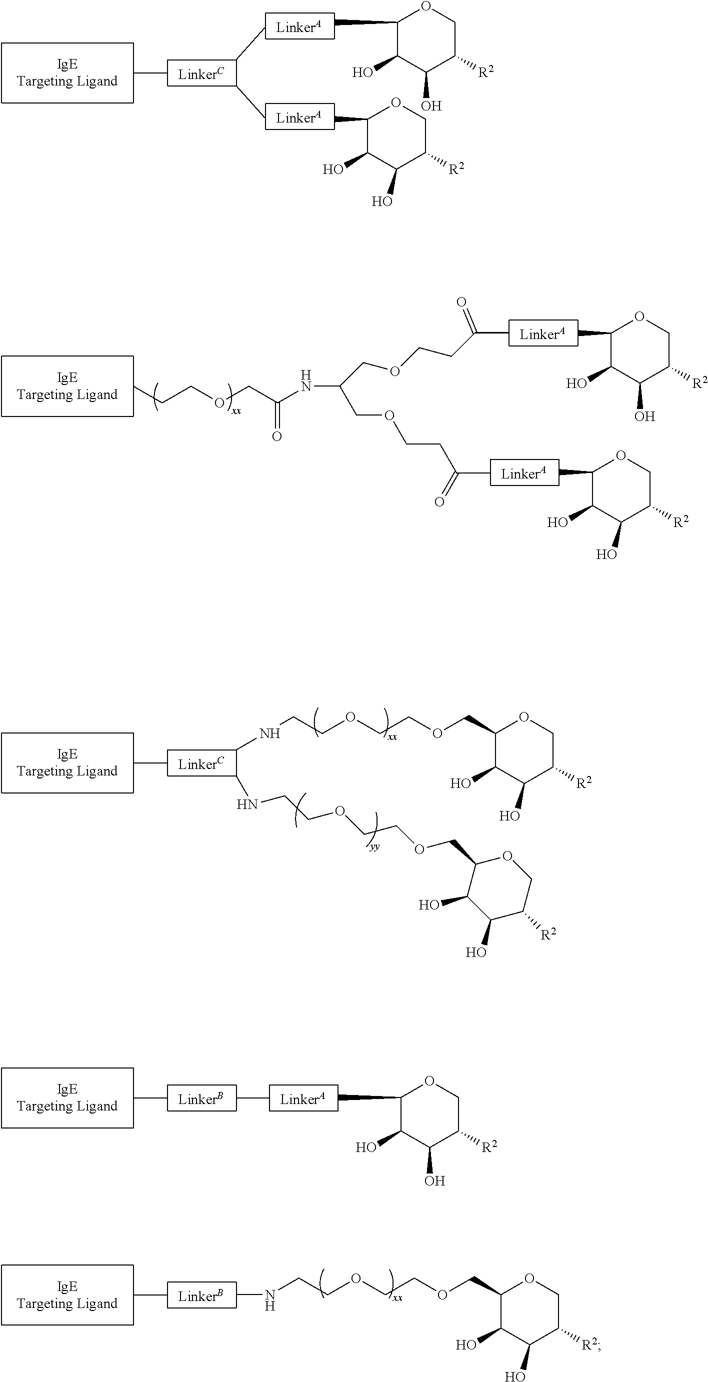

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

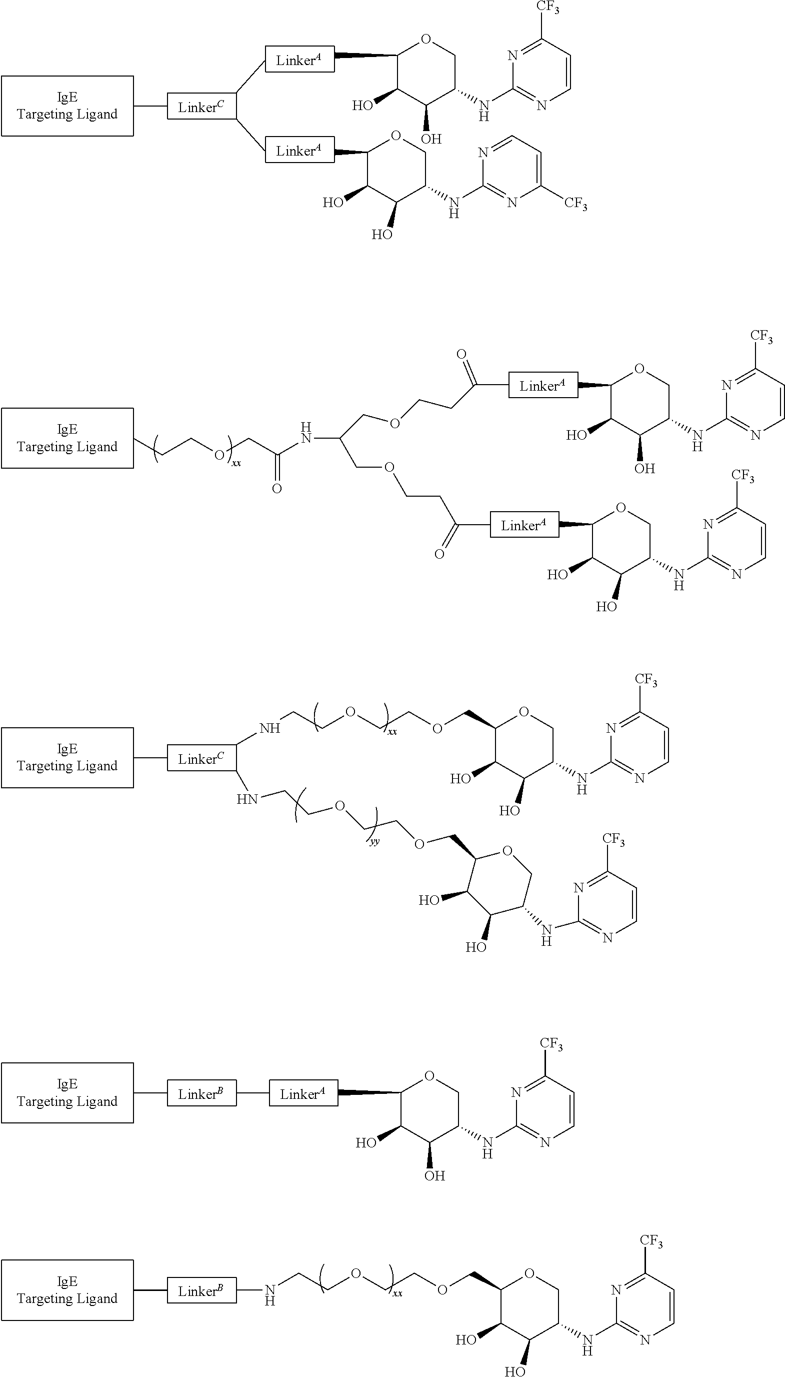

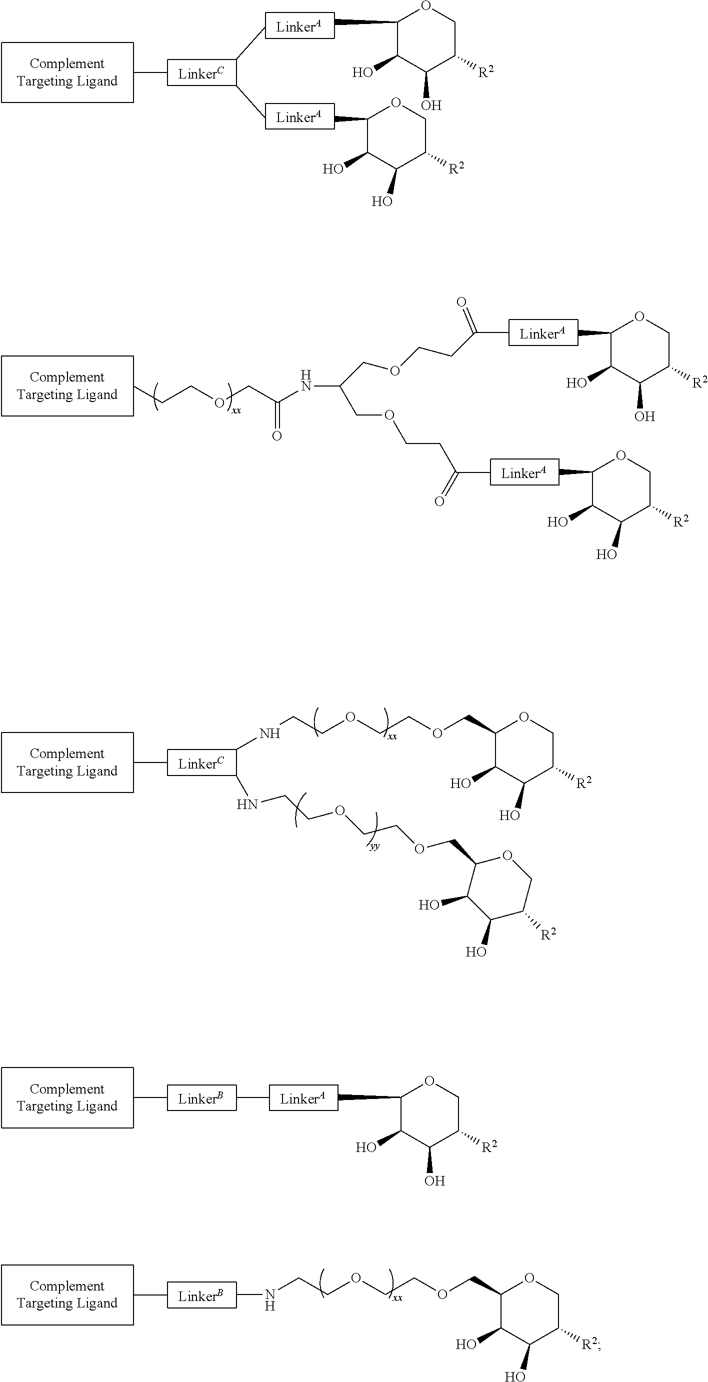

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

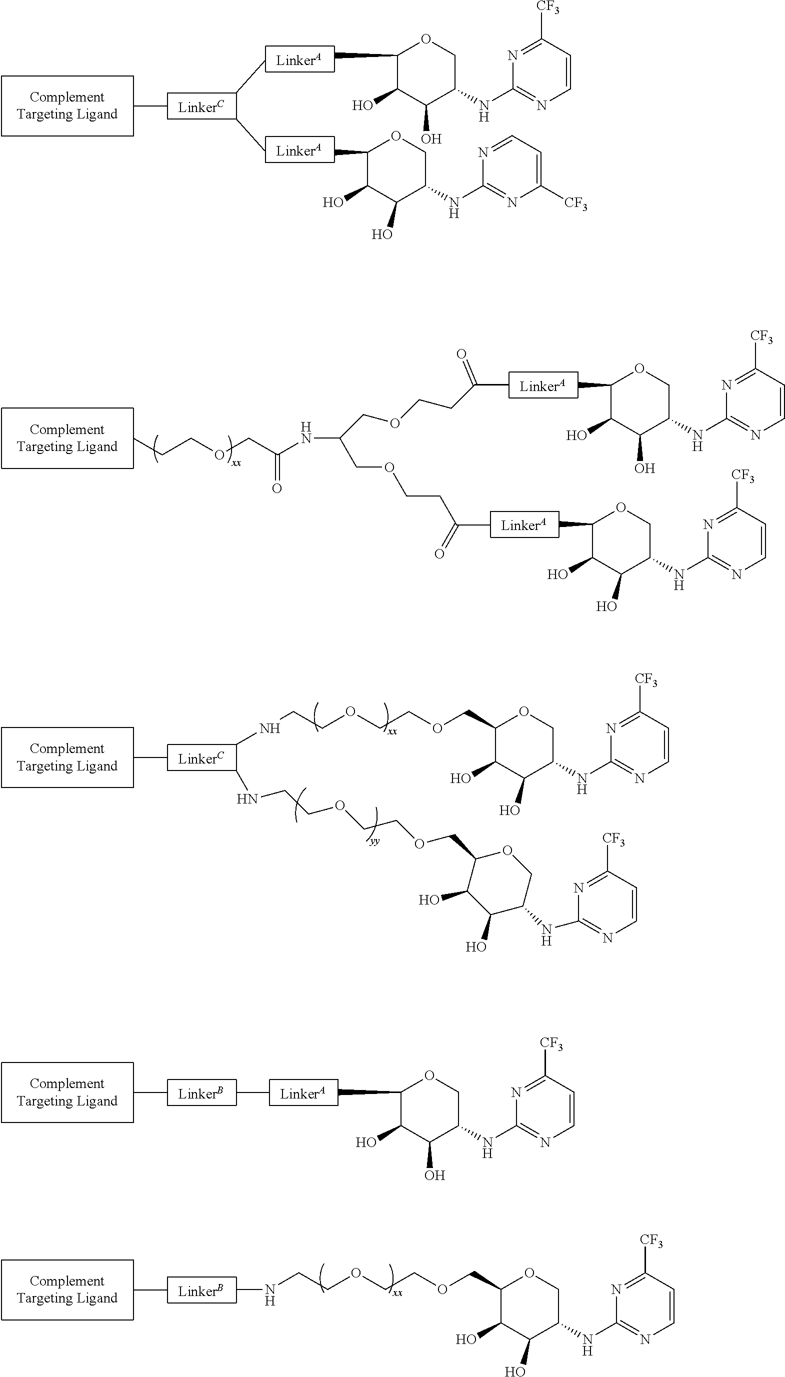

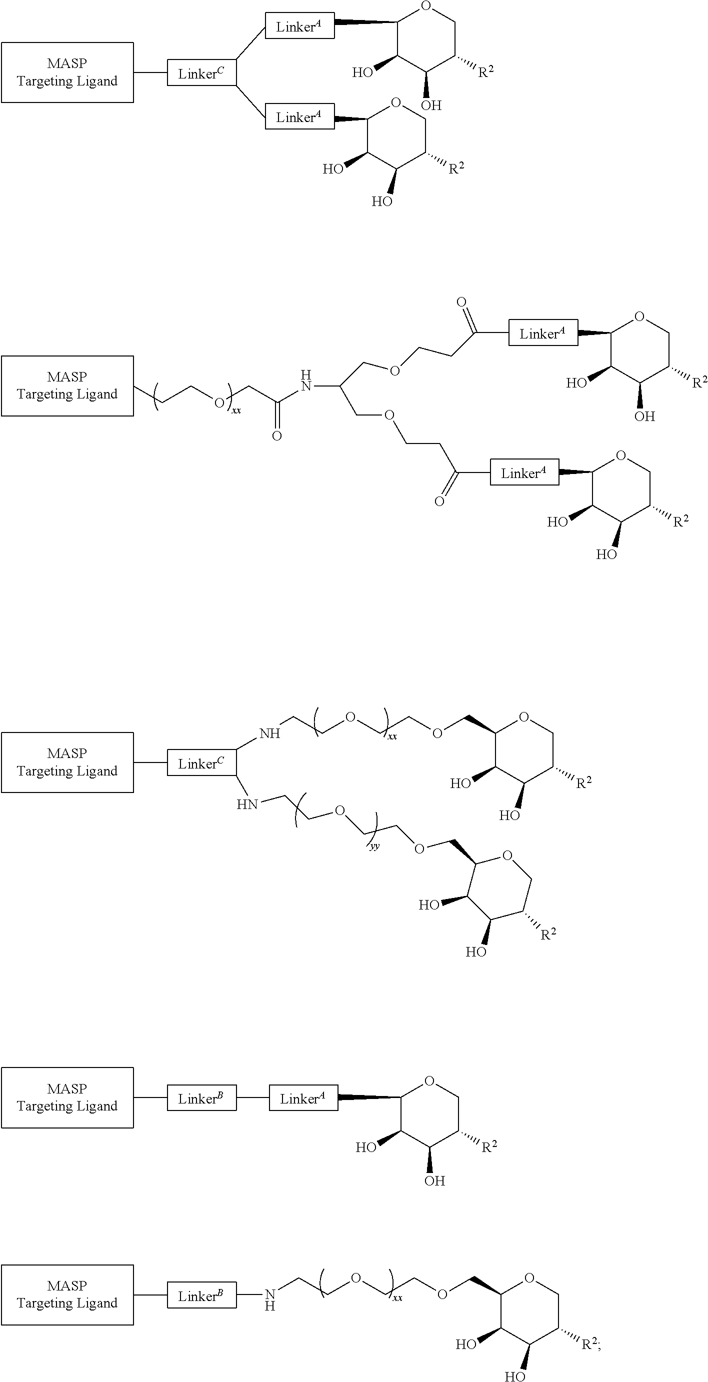

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

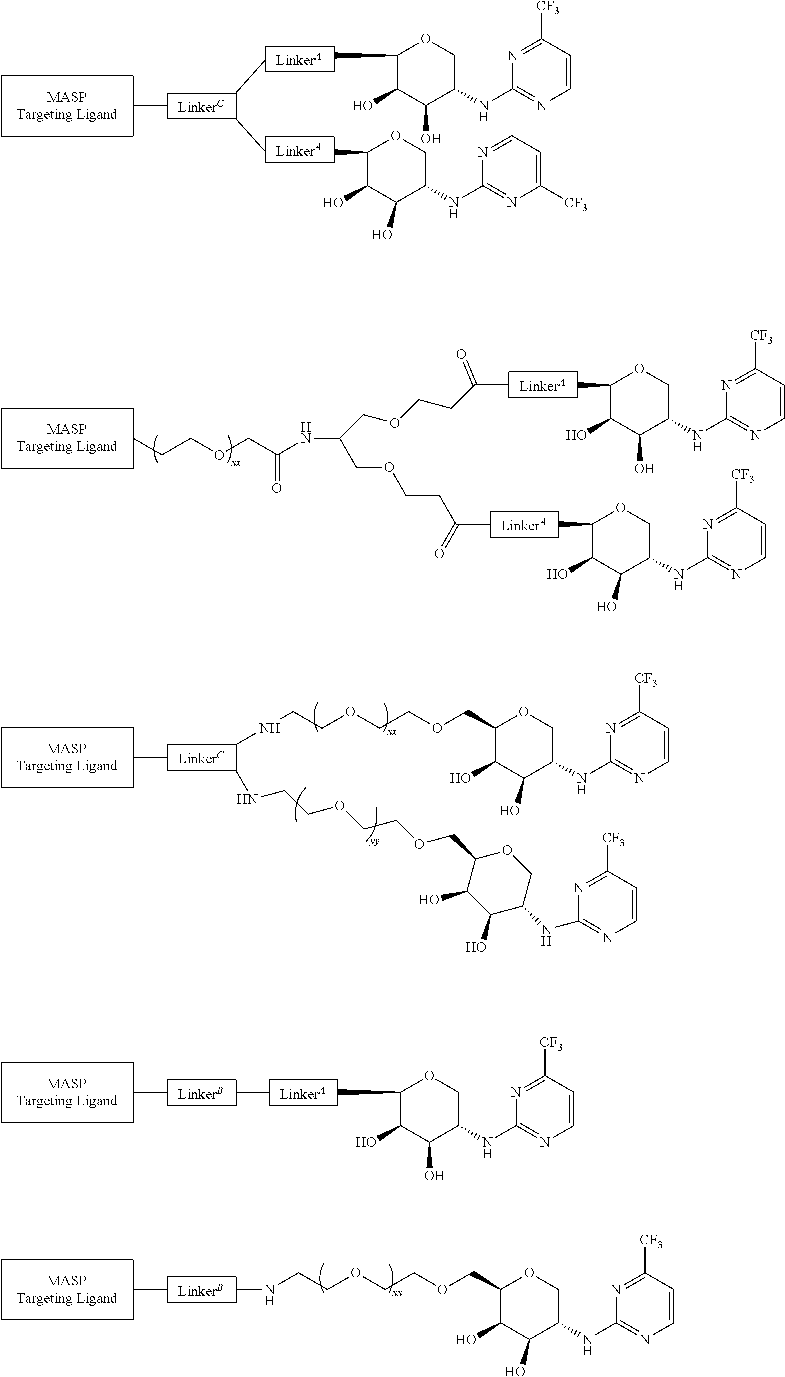

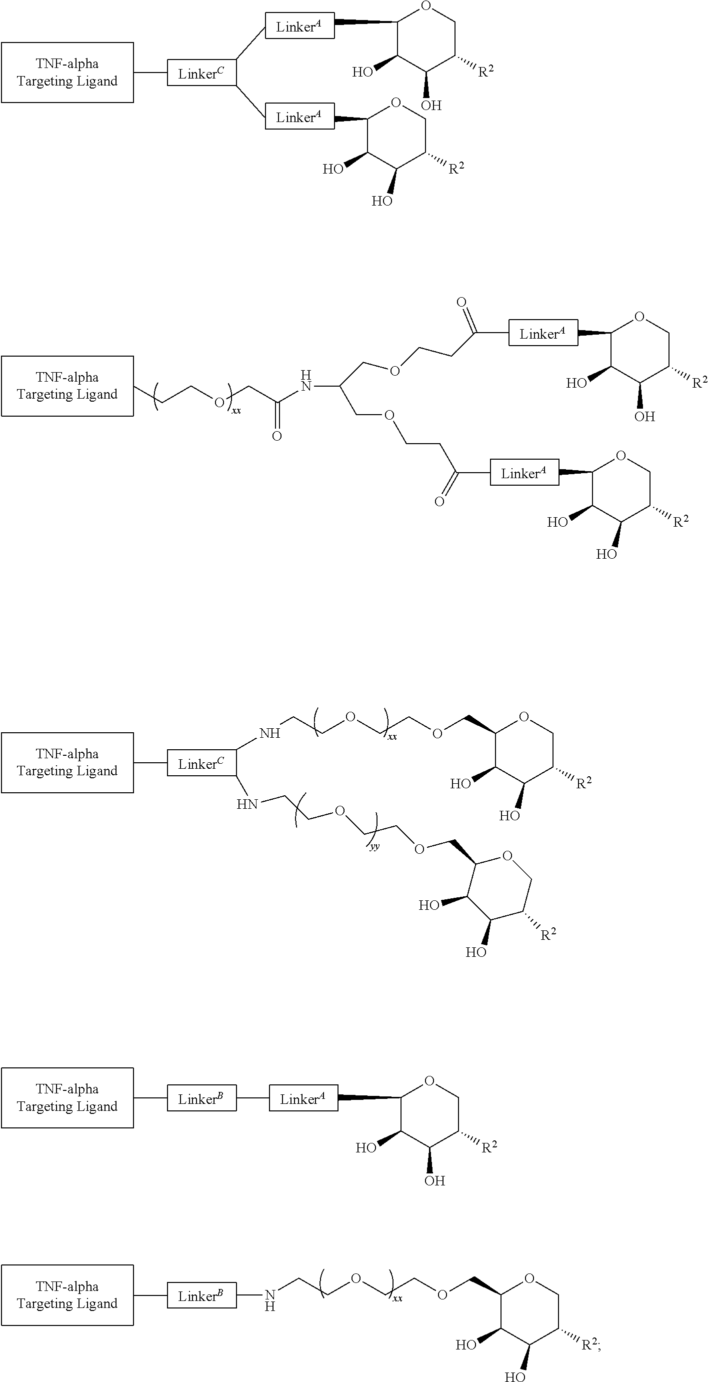

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

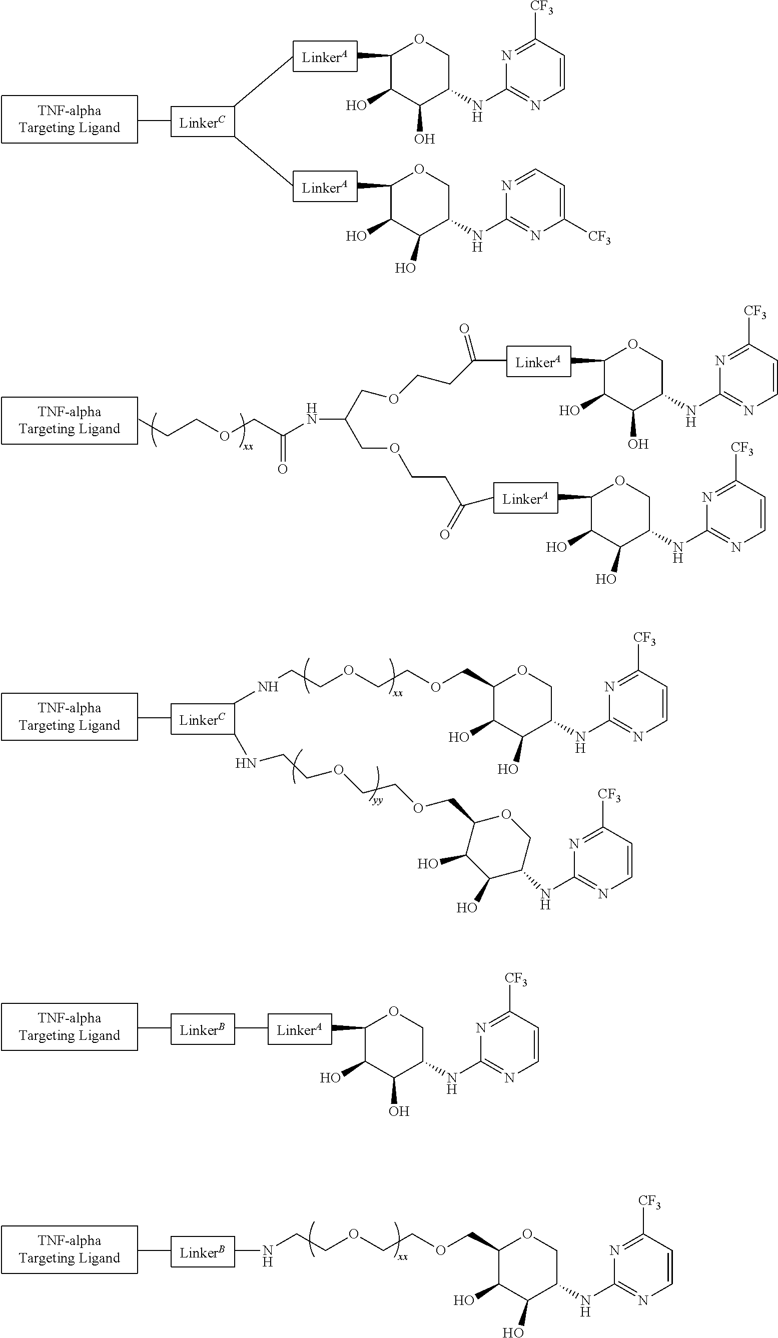

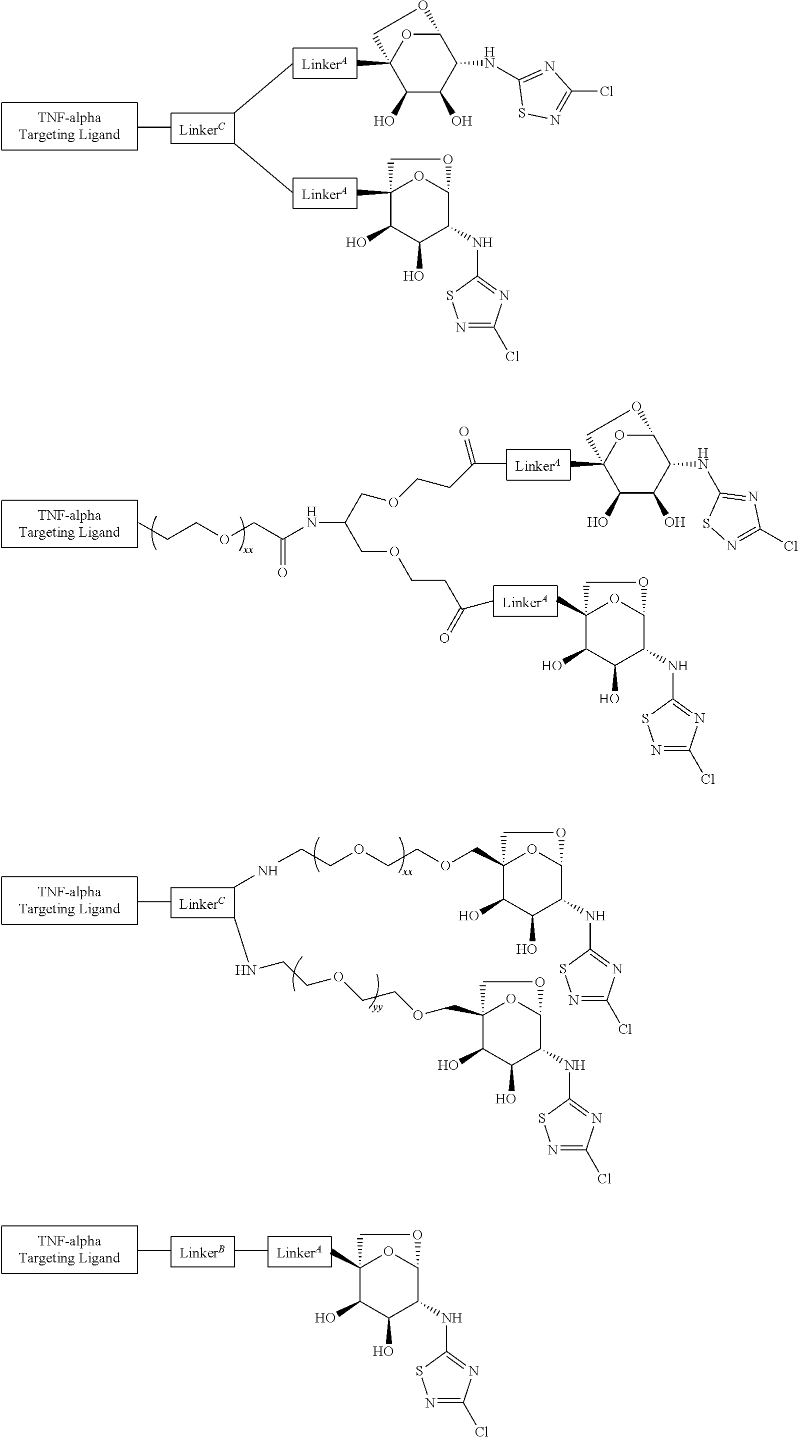

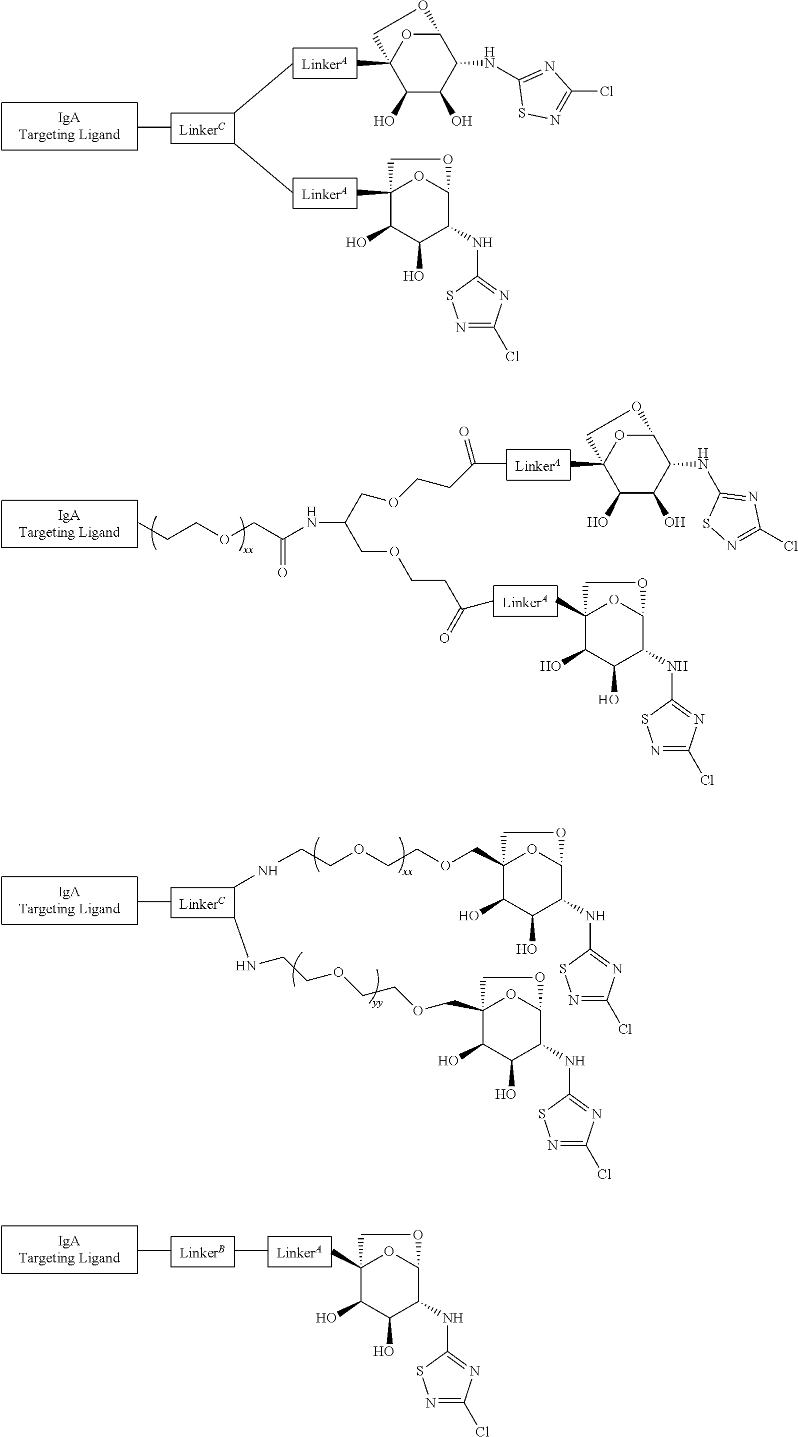

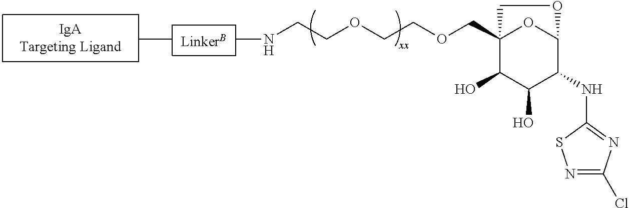

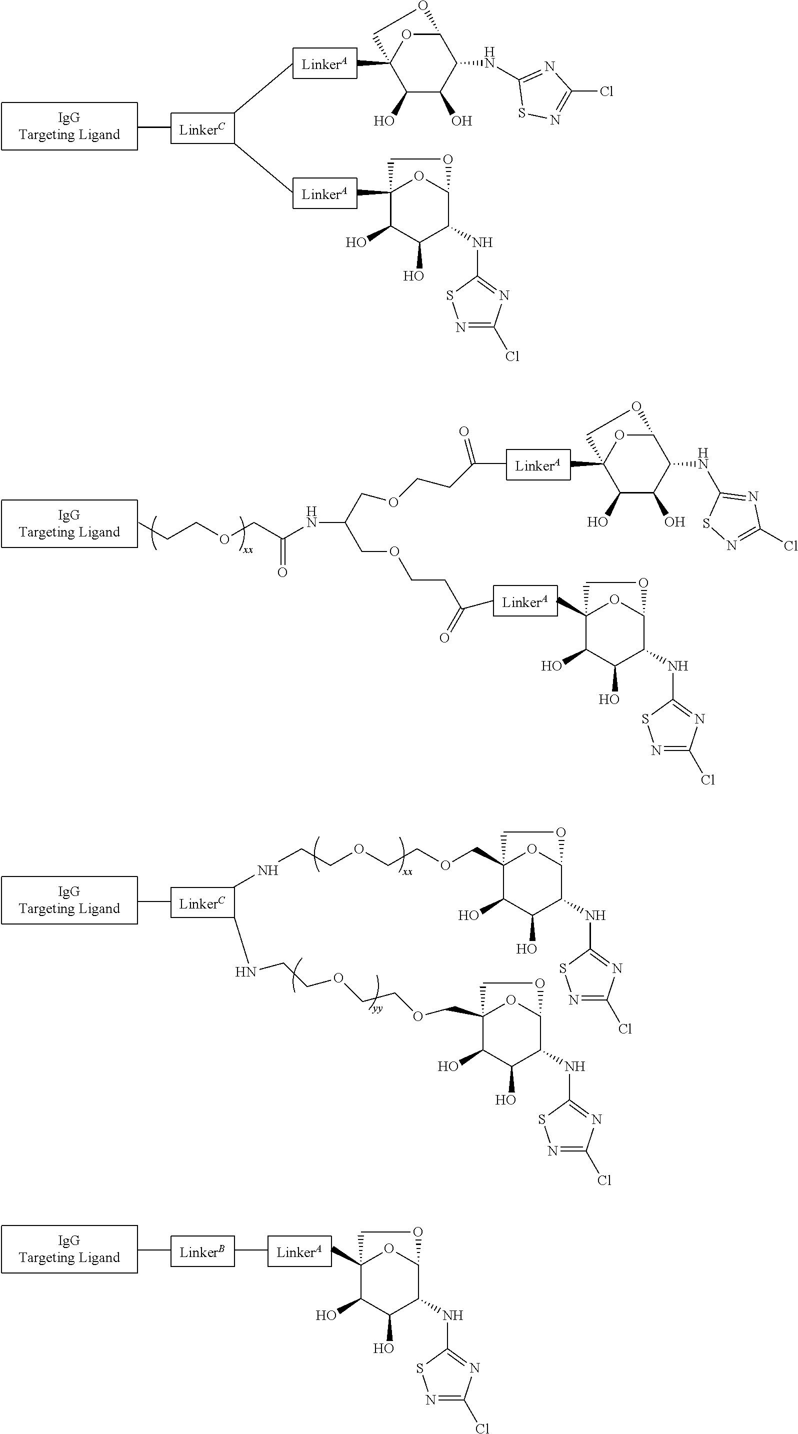

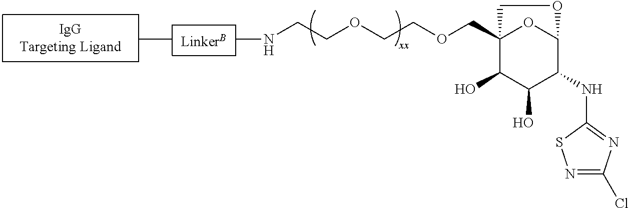

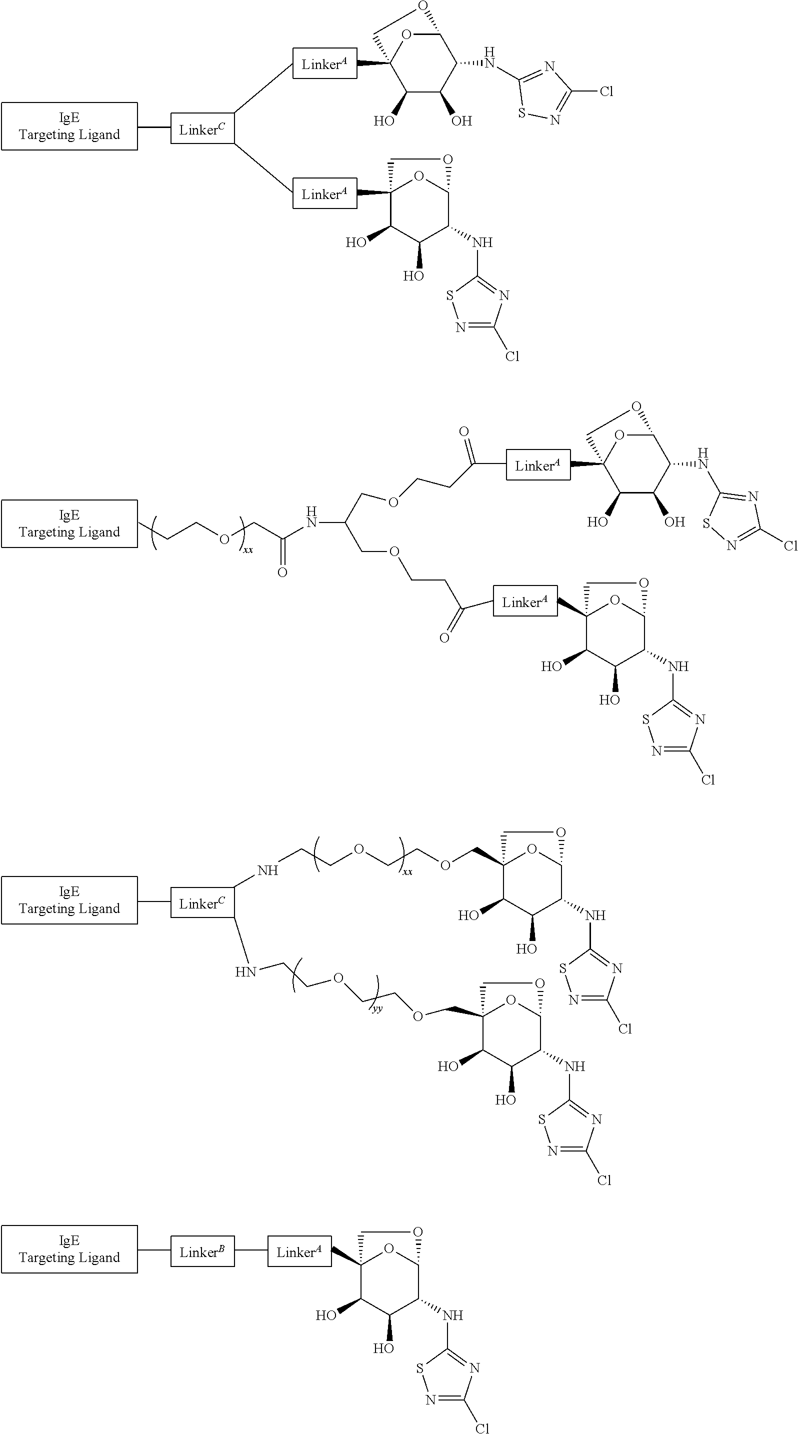

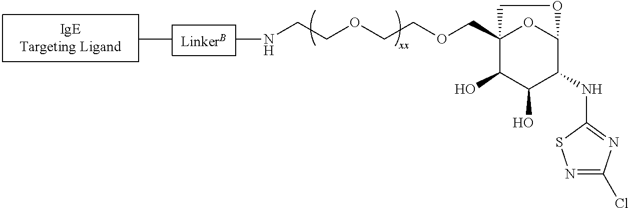

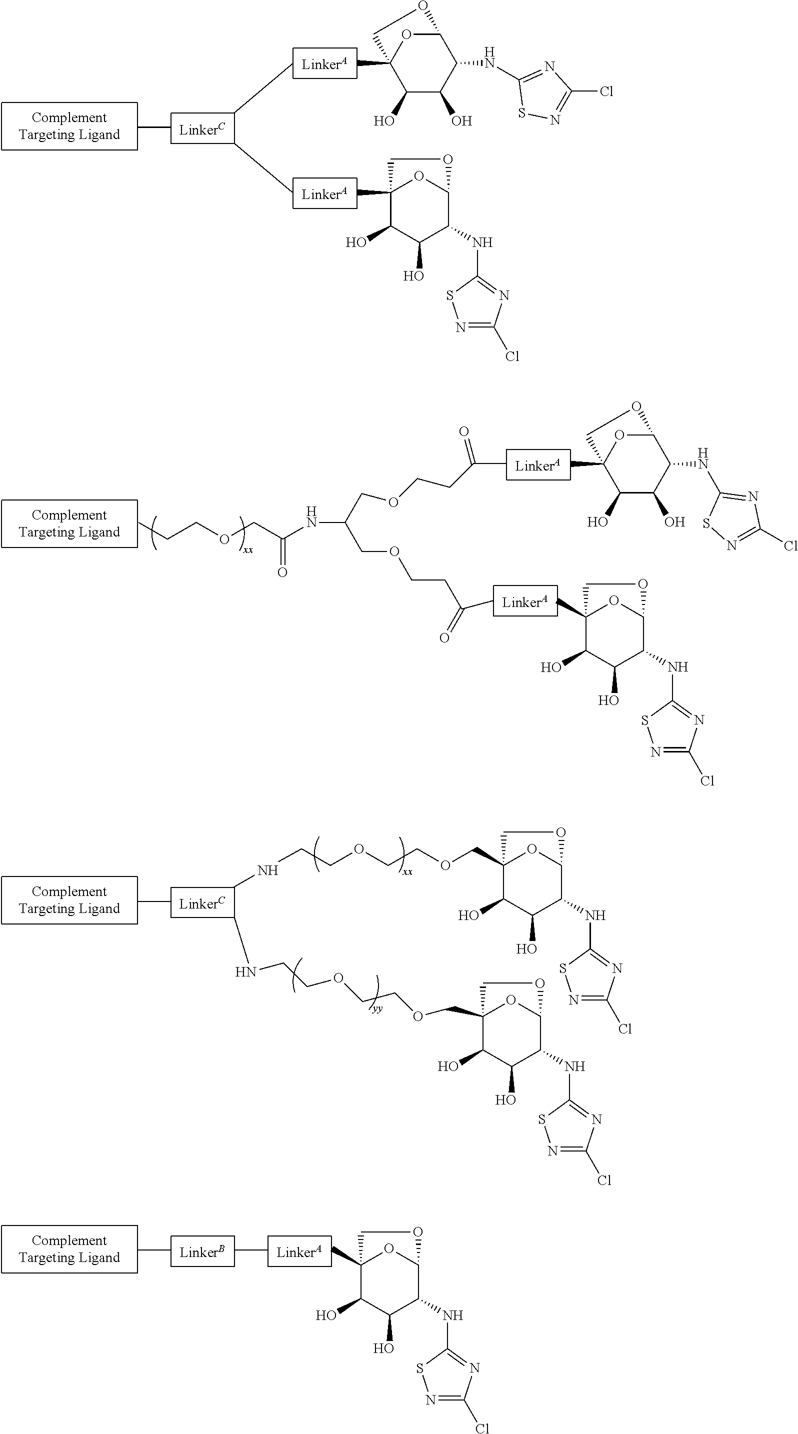

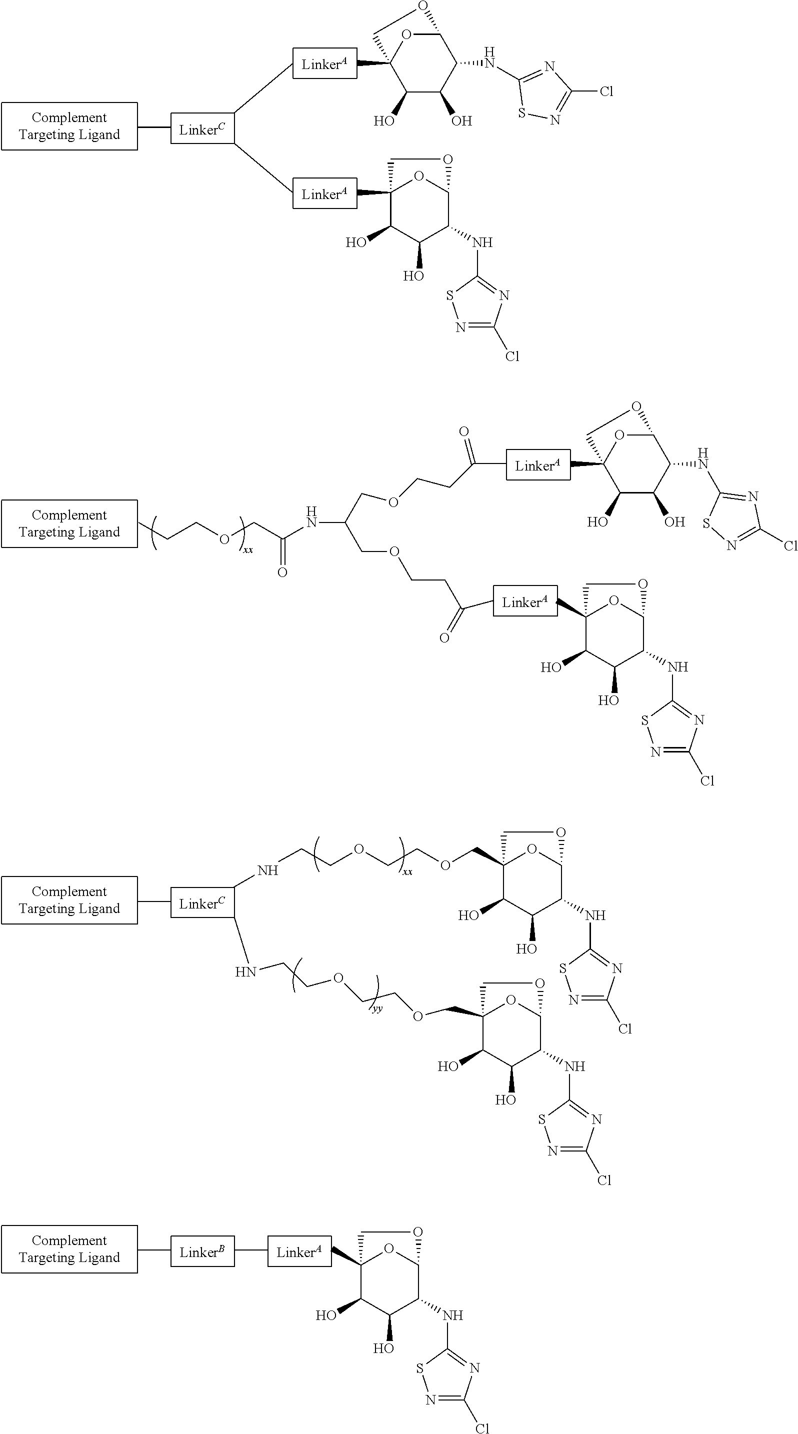

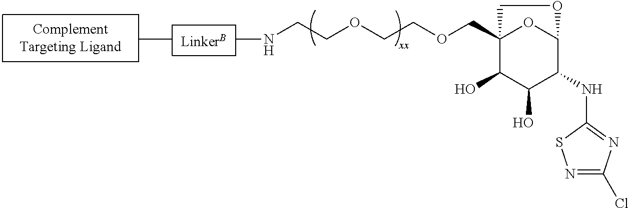

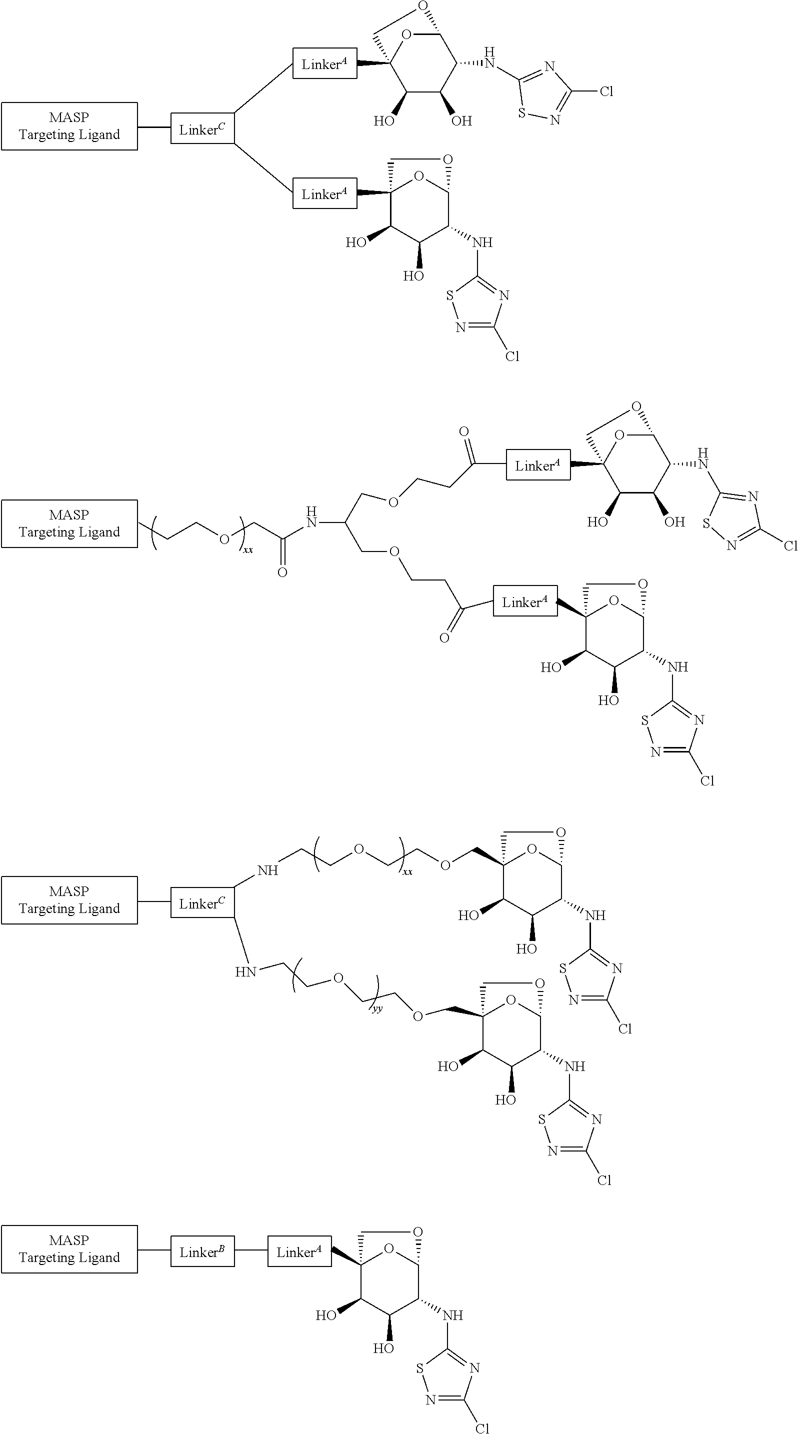

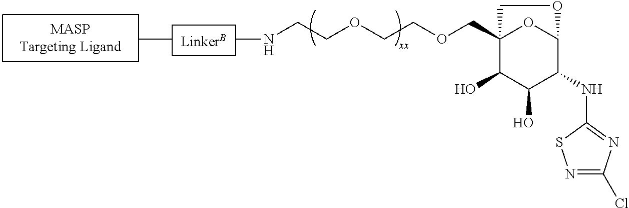

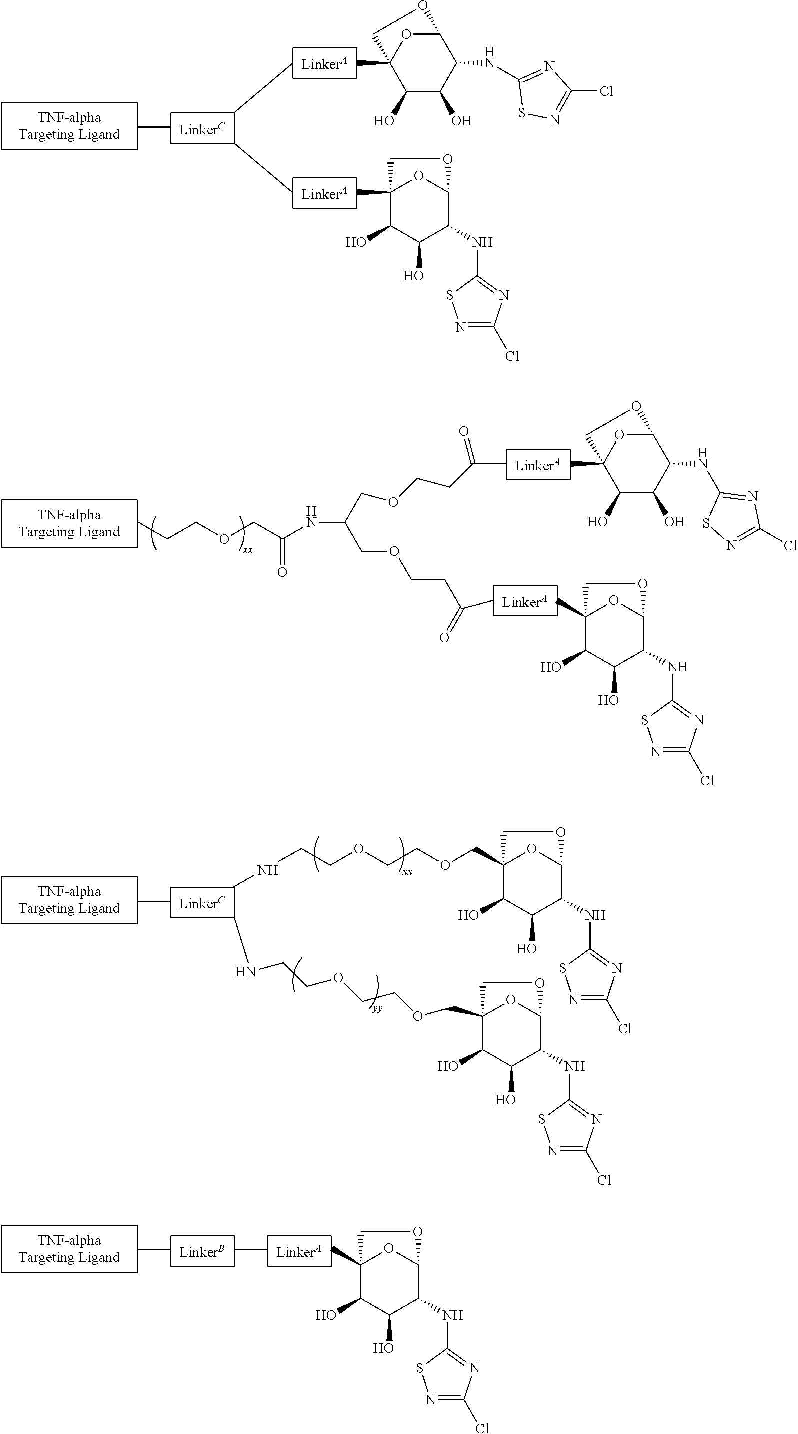

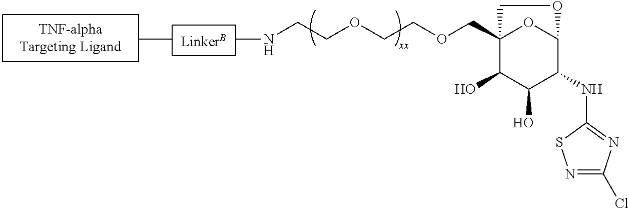

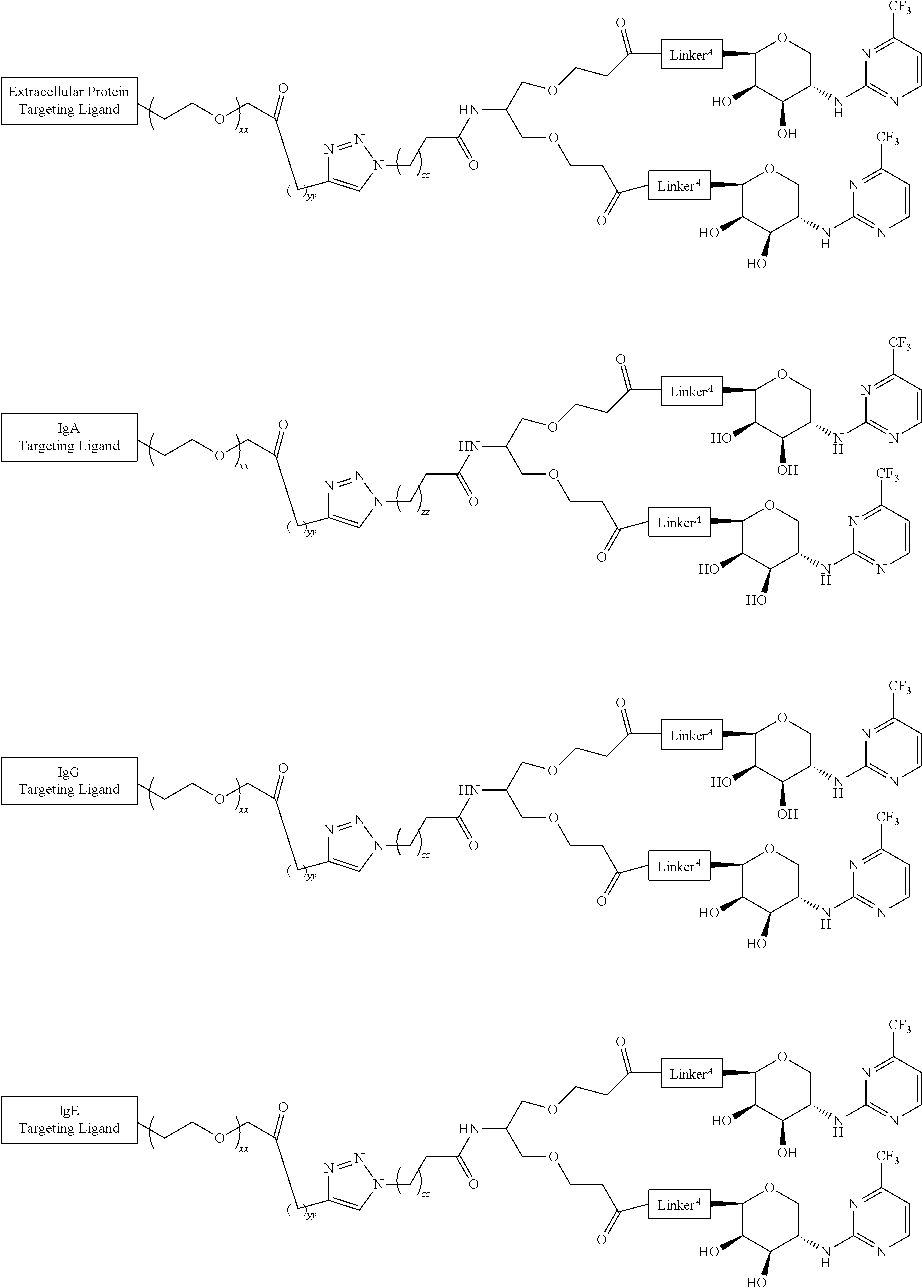

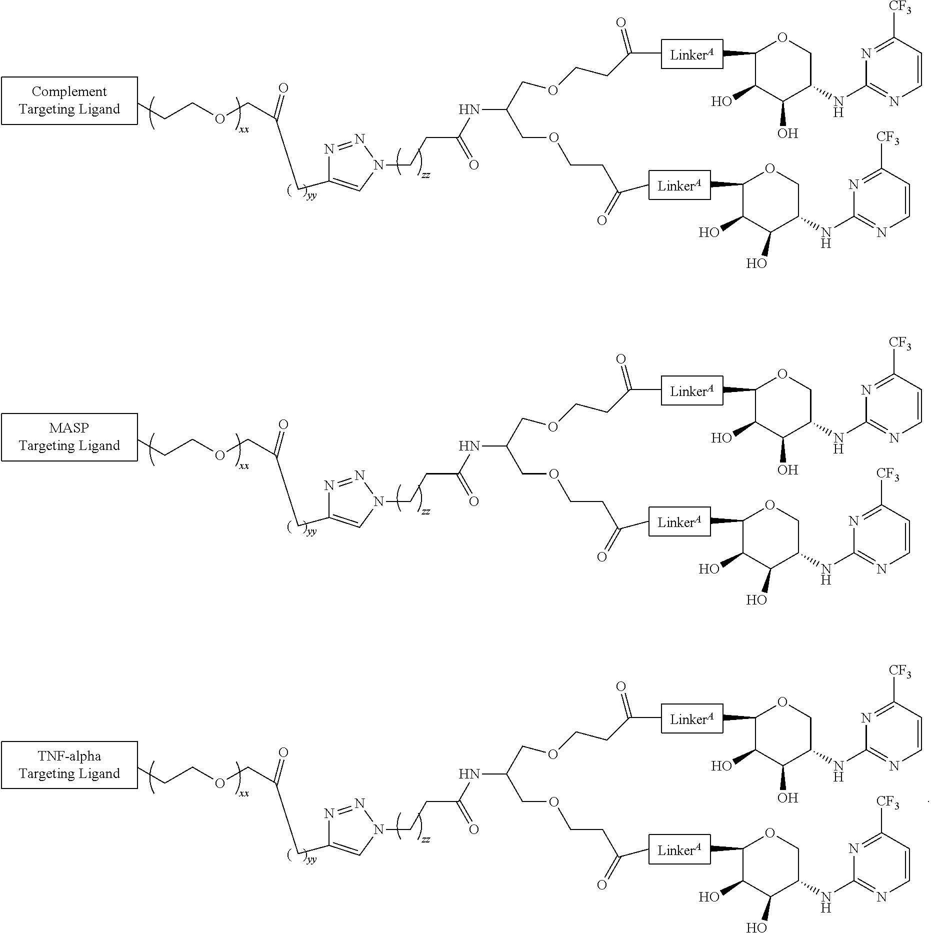

In certain embodiments, an ASGPR ligand useful for incorporation into a compound of Formula II is selected from:

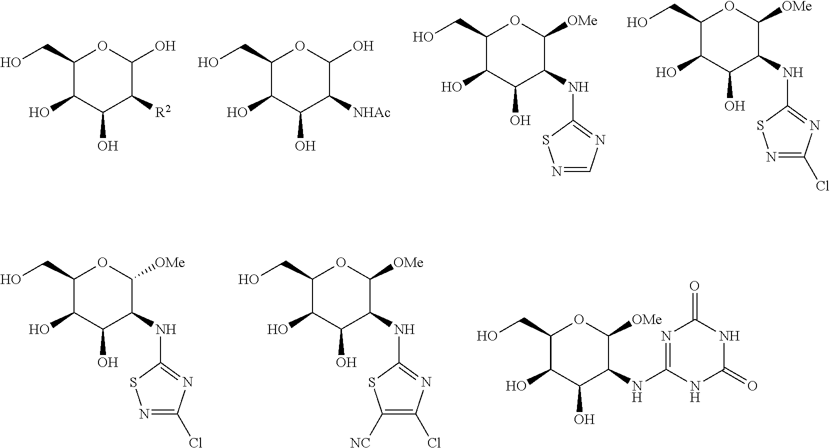

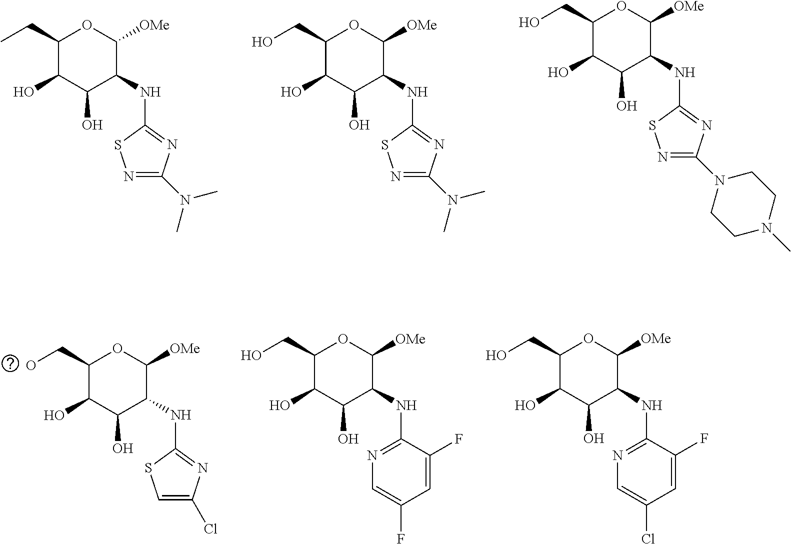 [SMILES: CC[C@H]1O[C@H](OC)[C@@H](Nc2nc(N(C)C)ns2)[C@@H](O)[C@H]1O ; CO[C@@H]1O[C@H](CO)[C@H](O)[C@H](O)[C@@H]1Nc1nc(N(C)C)ns1 ; CO[C@@H]1O[C@H](CO)[C@H](O)[C@H](O)[C@@H]1Nc1nc(N2CCN(C)CC2)ns1 ; CO[C@@H]1O[C@H](CO)[C@H](O)[C@H](O)[C@H]1Nc1nc(Cl)cs1 ; CO[C@@H]1O[C@H](CO)[C@H](O)[C@H](O)[C@@H]1Nc1ncc(F)cc1F ; CO[C@@H]1O[C@H](CO)[C@H](O)[C@H](O)[C@@H]1Nc1ncc(Cl)cc1F]

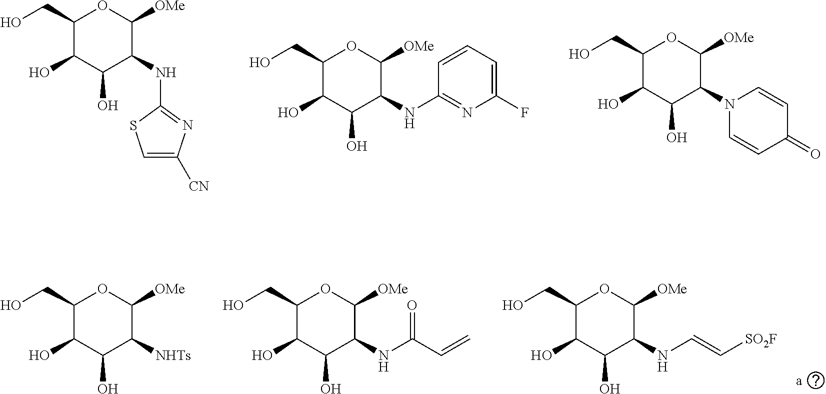

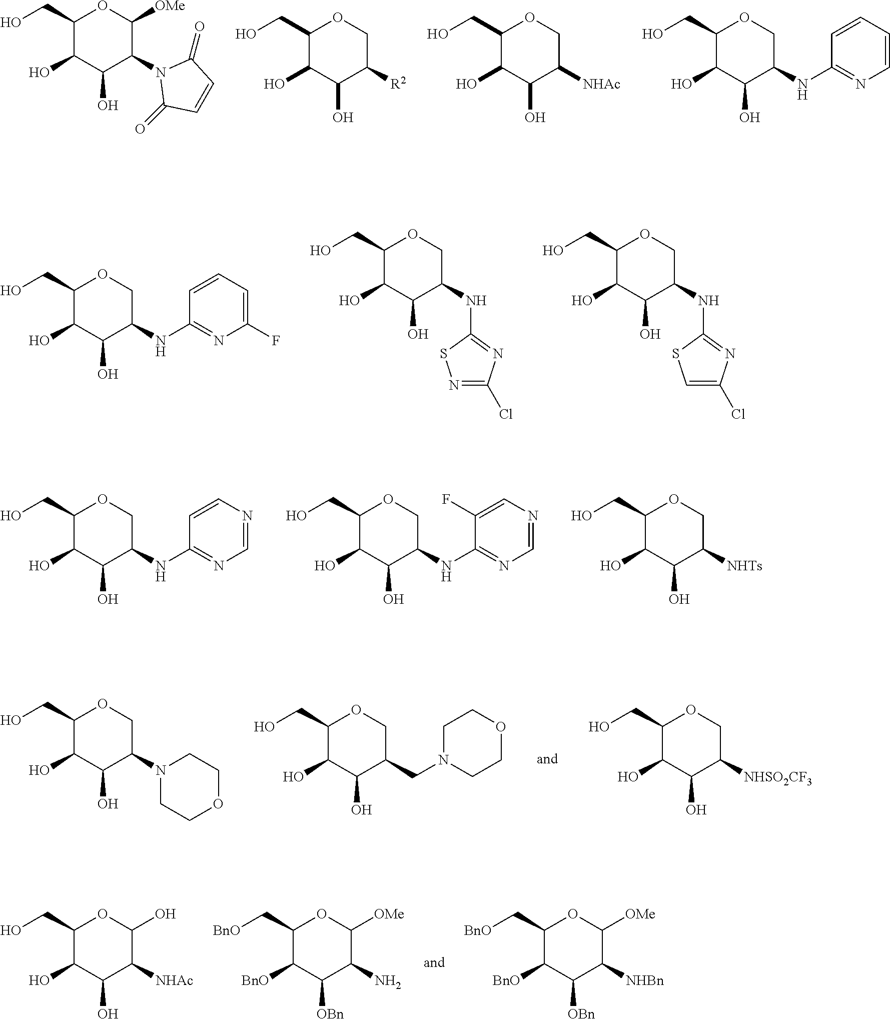

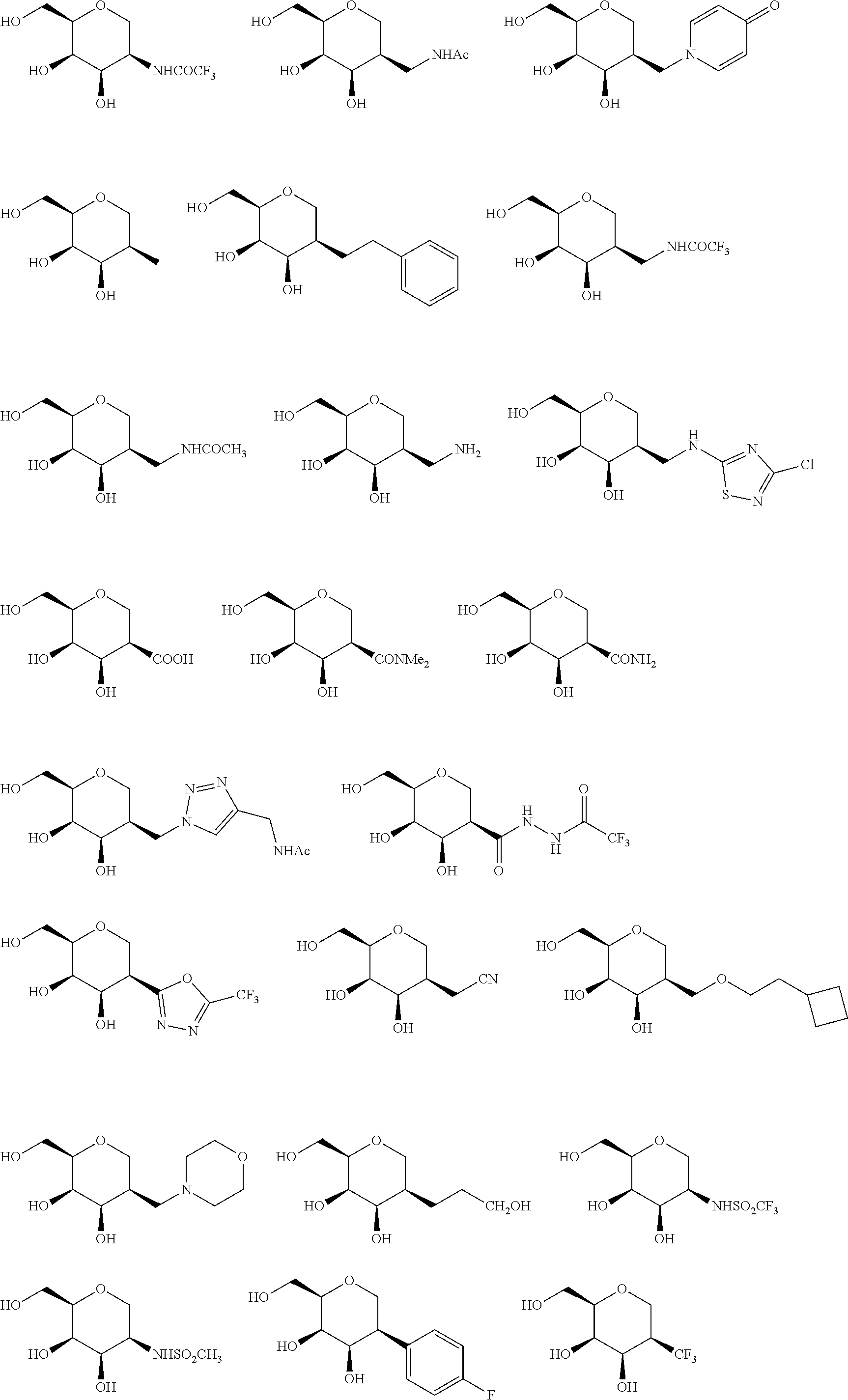

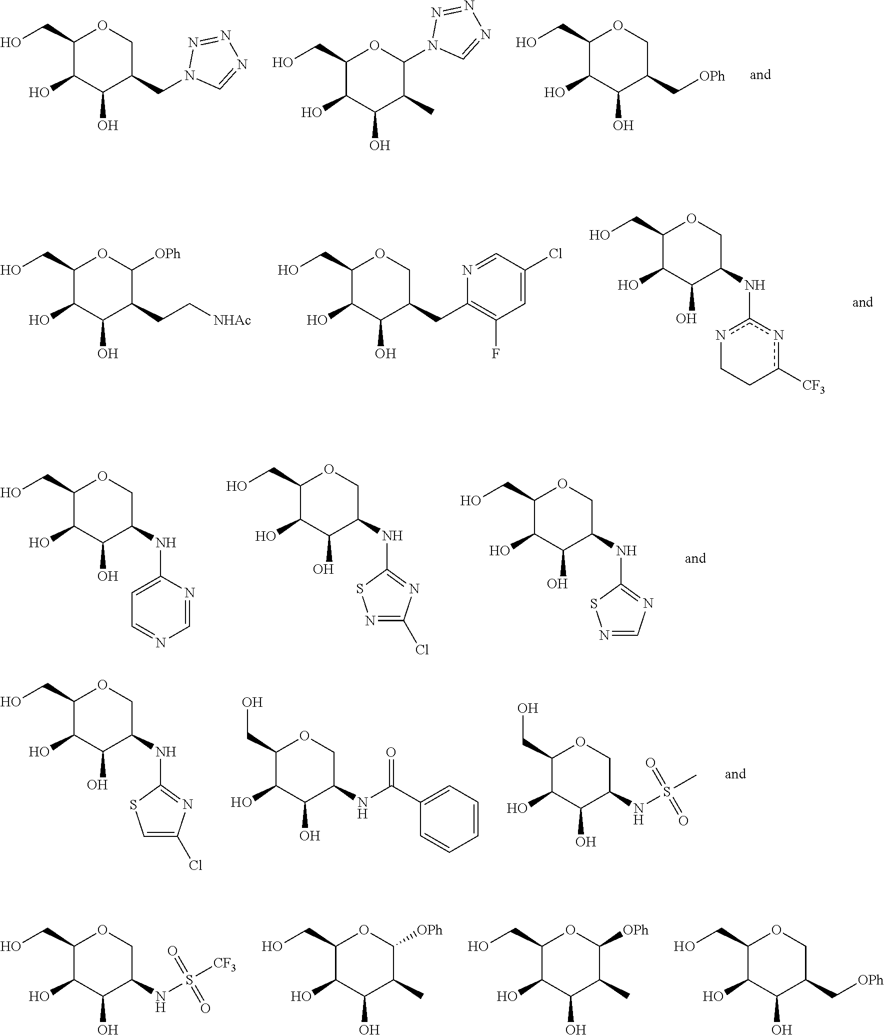

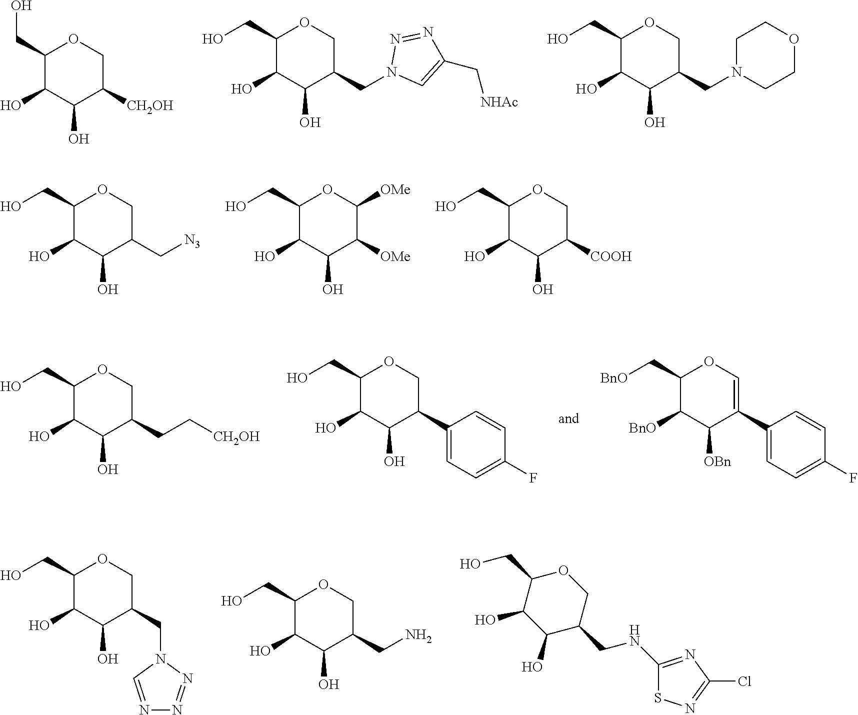

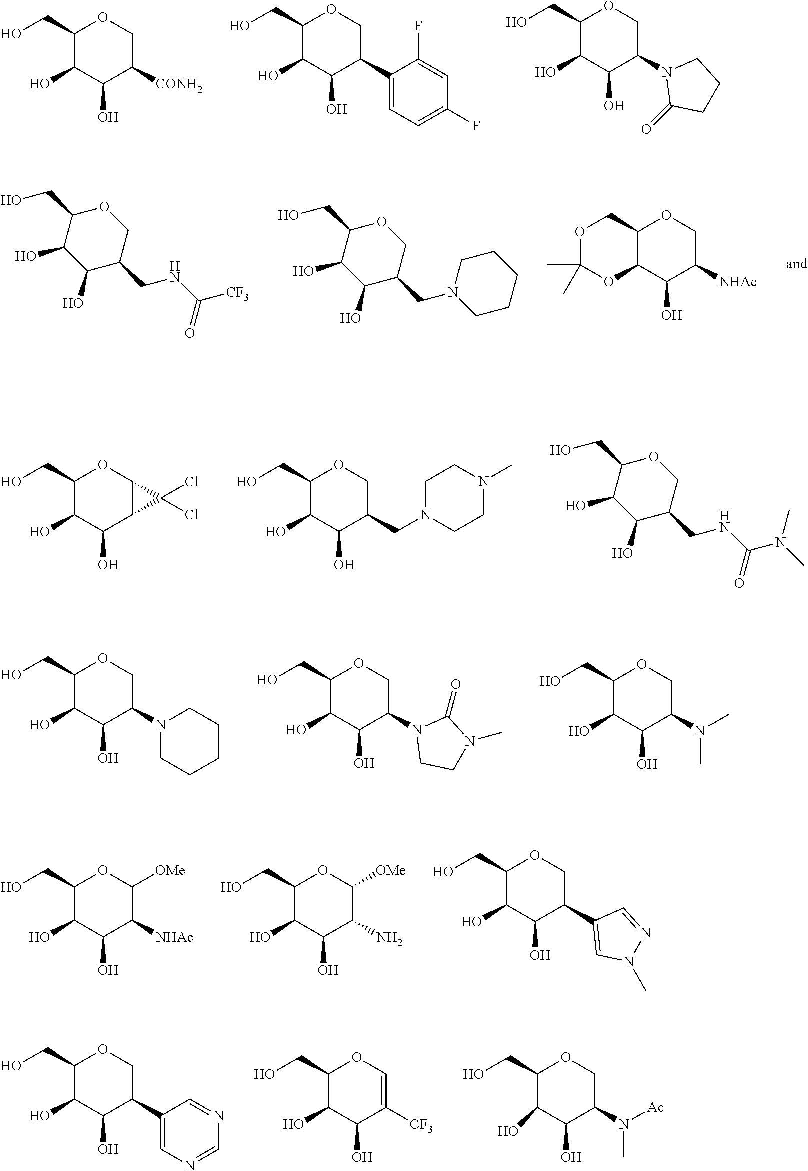

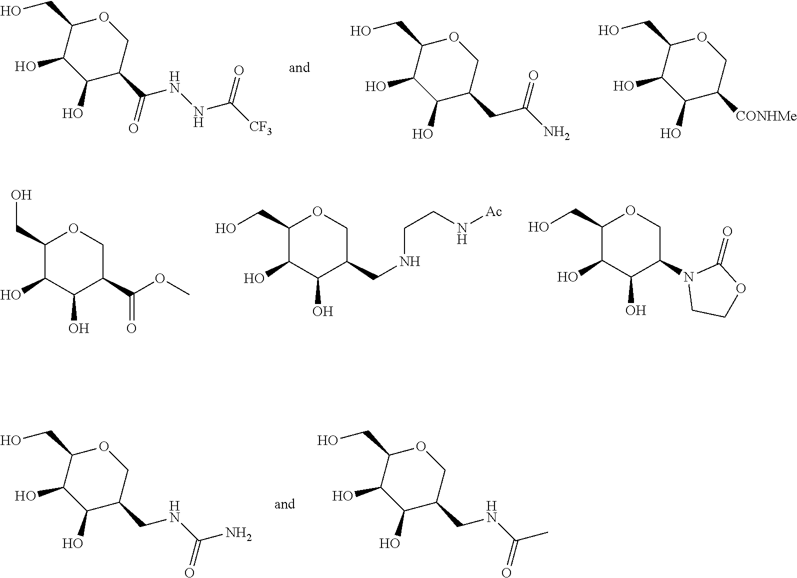

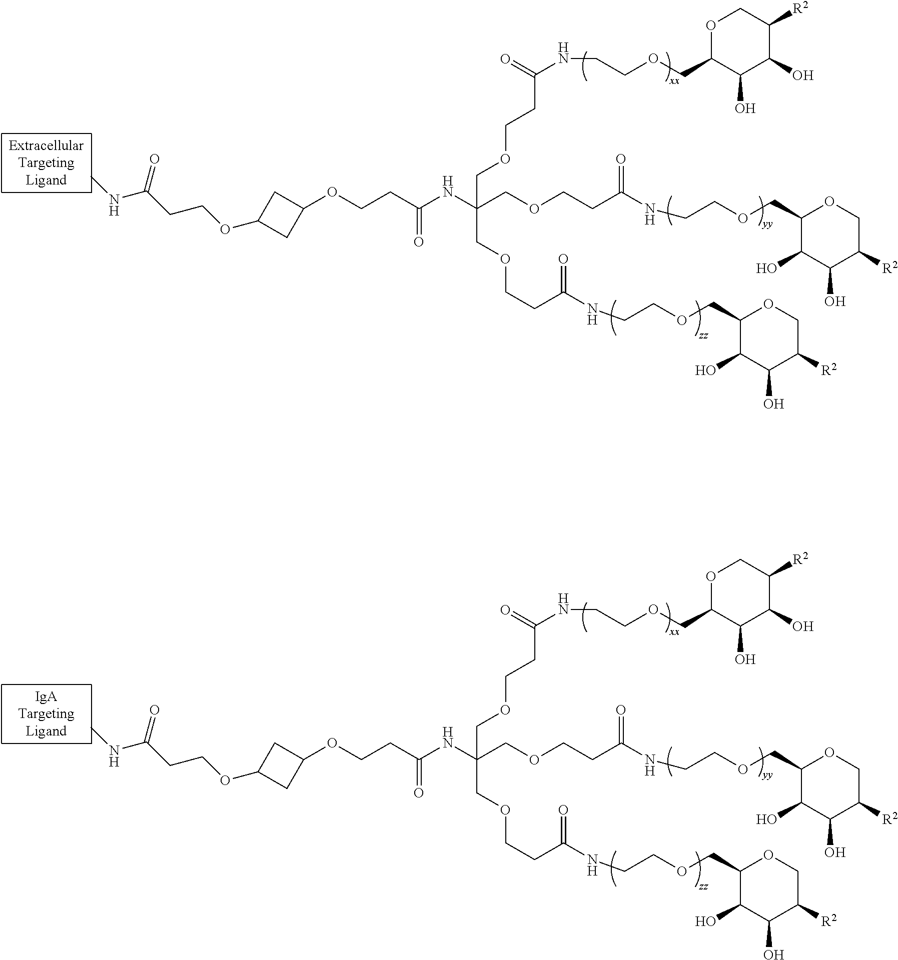

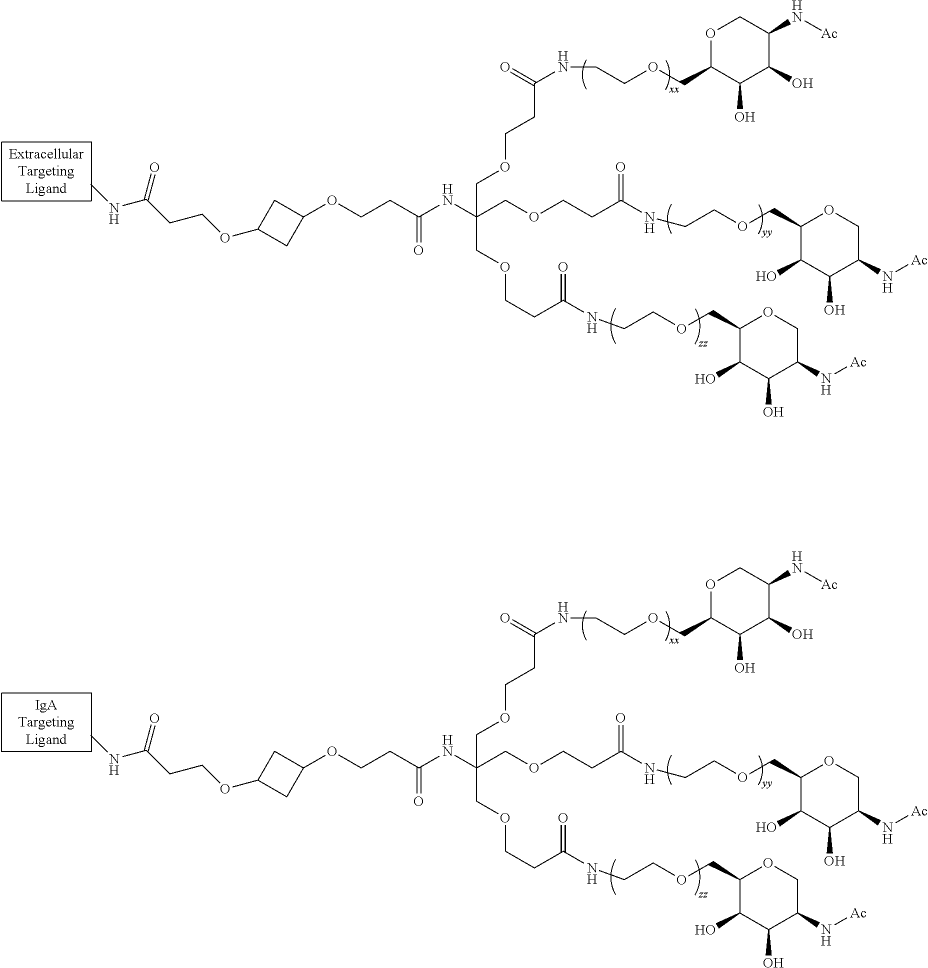

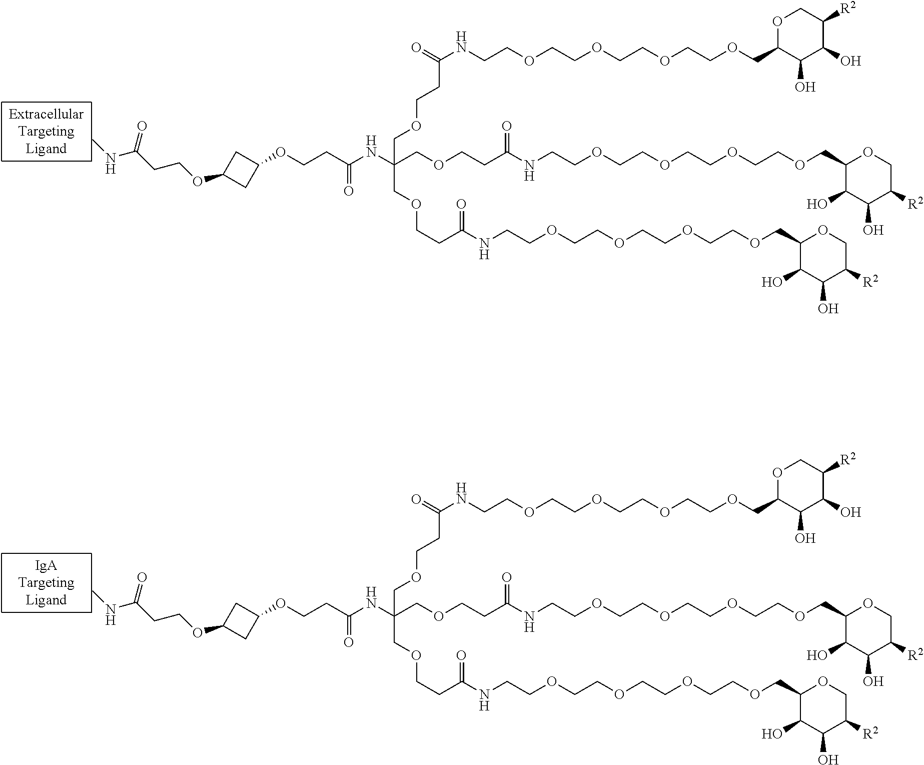

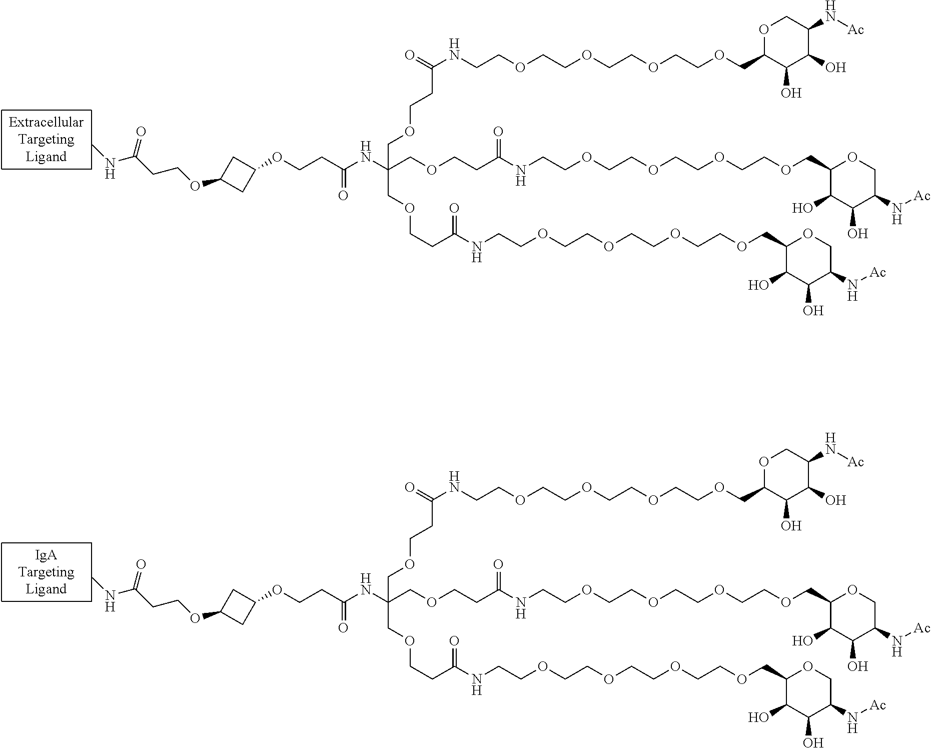 [SMILES: CNC(=O)CCO[C@H]1C[C@H](OCCC(=O)NC(COCCC(=O)NCCOCCOCCOCCOC[C@H]2OC[C@@H](NC(C)=O)[C@@H](O)[C@H]2O)(COCCC(=O)NCCOCCOCCOCCOC[C@H]2OC[C@@H](NC(C)=O)[C@@H](O)[C@H]2O)COCCC(=O)NCCOCCOCCOCCOC[C@H]2OC[C@@H](NC(C)=O)[C@@H](O)[C@H]2O)C1 ; CNC(=O)CCO[C@H]1C[C@H](OCCC(=O)NC(COCCC(=O)NCCOCCOCCOCCOC[C@H]2OC[C@@H](NC(C)=O)[C@@H](O)[C@H]2O)(COCCC(=O)NCCOCCOCCOCCOC[C@H]2OC[C@@H](NC(C)=O)[C@@H](O)[C@H]2O)COCCC(=O)NCCOCCOCCOCCOC[C@H]2OC[C@@H](NC(C)=O)[C@@H](O)[C@H]2O)C1]

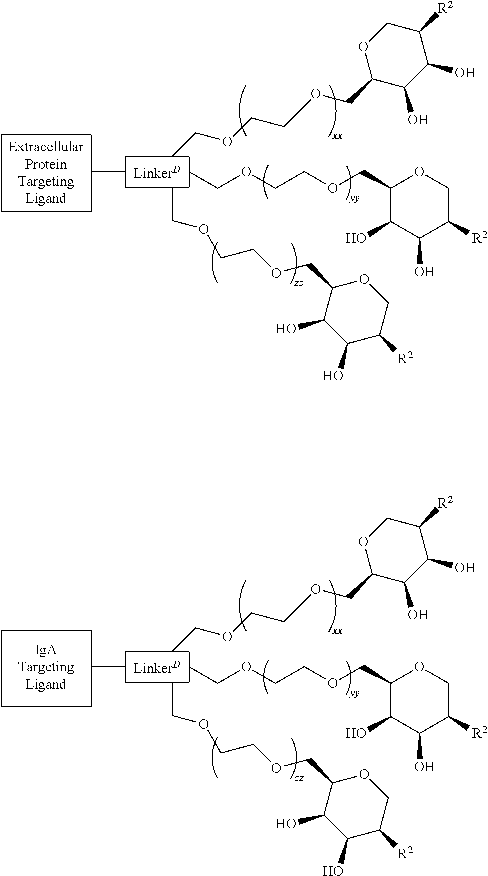

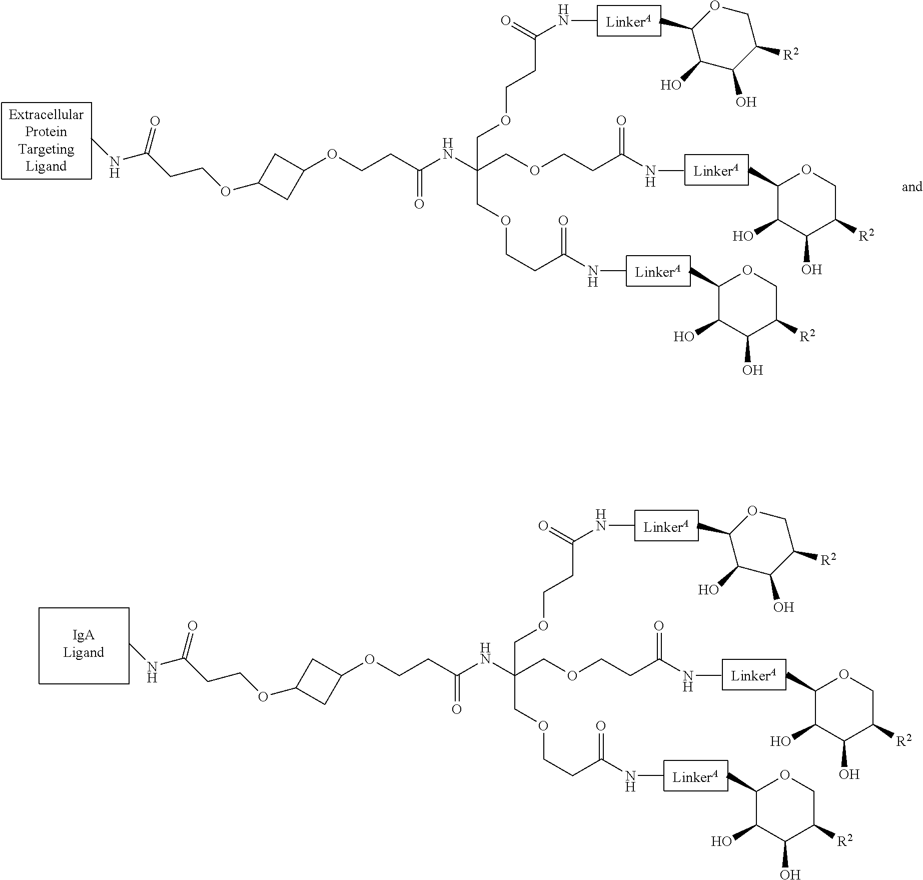

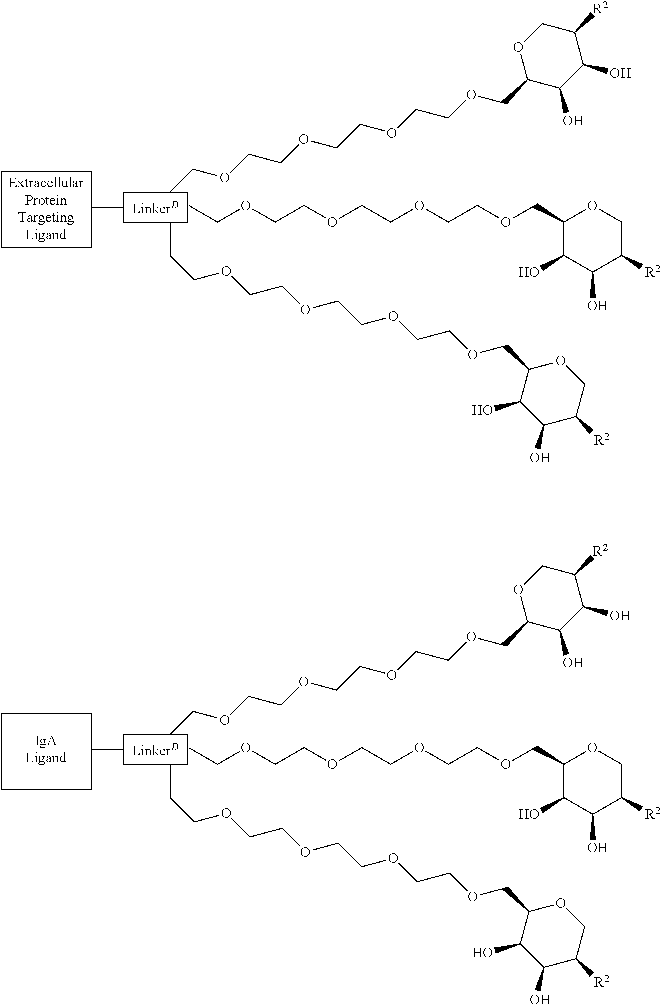

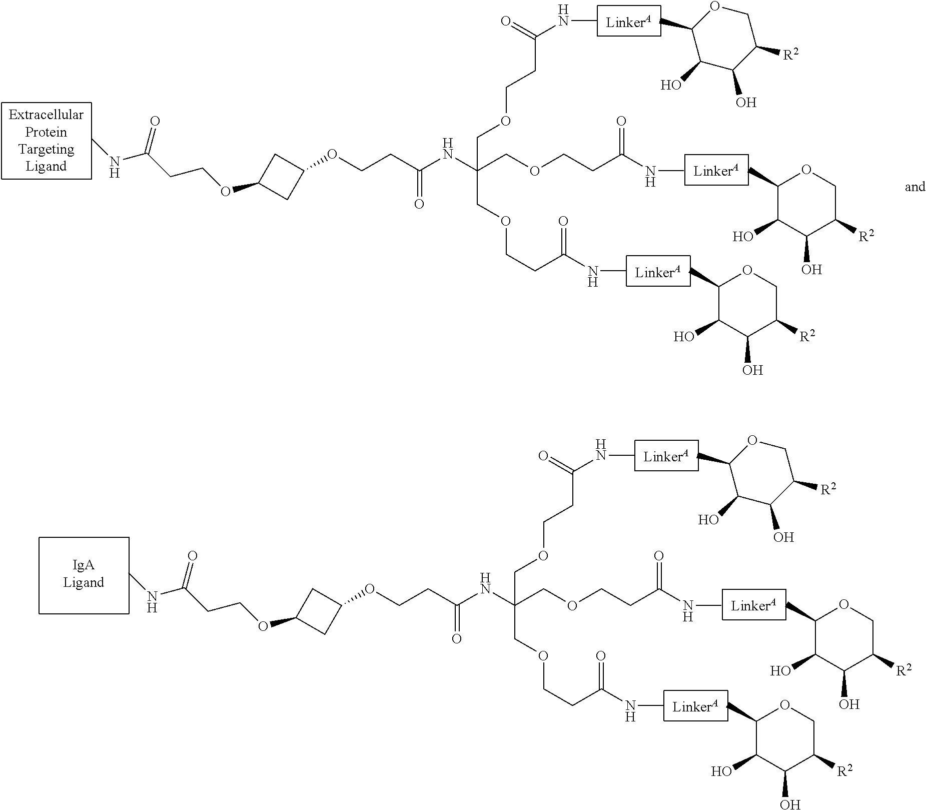

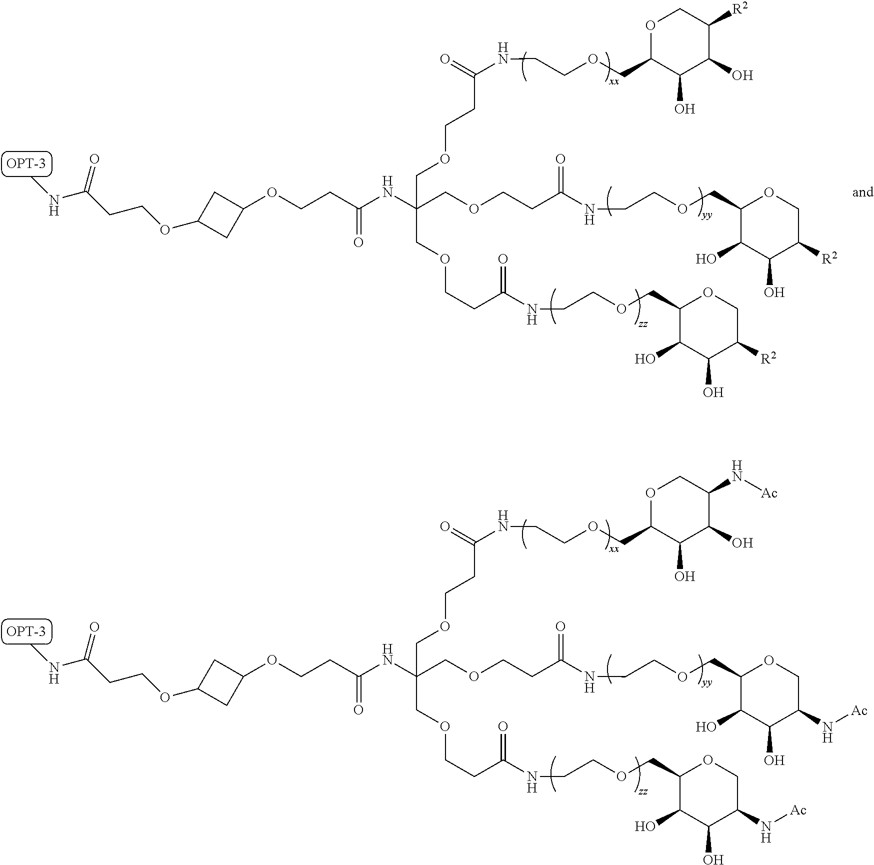

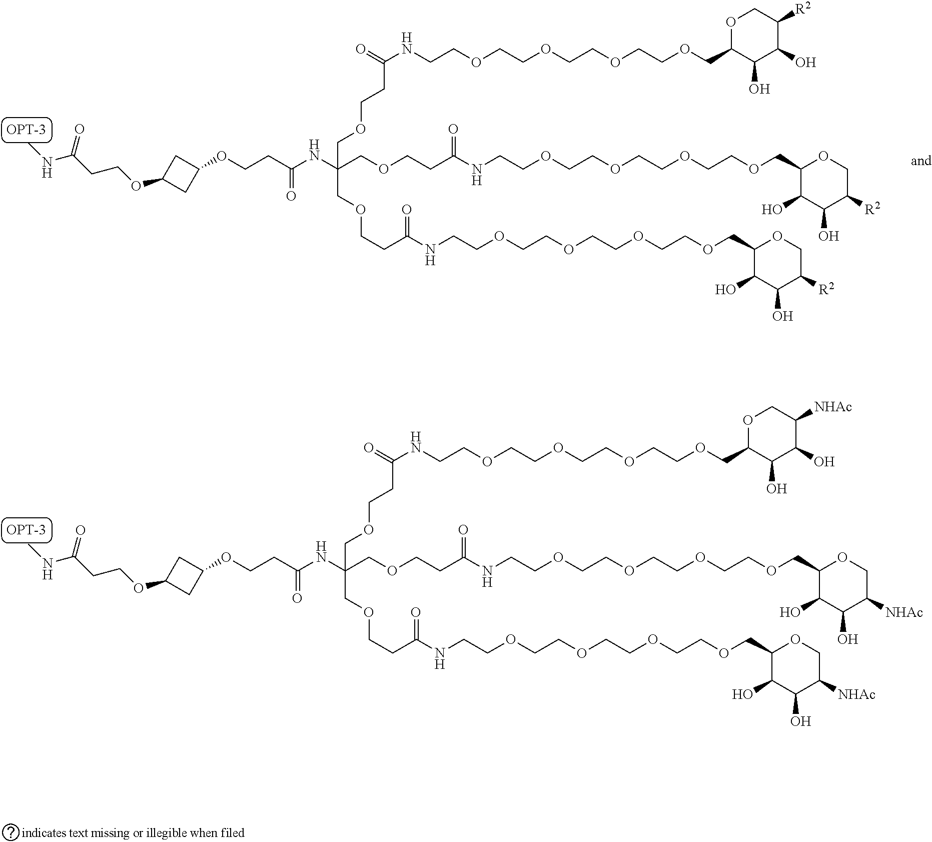

C. The ASGPR Ligand/Binding Moiety in Compounds of Formula II

In certain embodiments, in the compound of Formula II, R¹ is hydrogen.

In certain embodiments, in the compound of Formula II, R¹ is

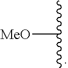 [SMILES: *OC]

In certain embodiments, in the compound of Formula II, R¹ is

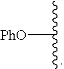 [SMILES: *Oc1ccccc1]

In certain embodiments, in the compound of Formula II, R¹ is

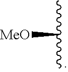

In certain embodiments, in the compound of Formula II, R¹ is

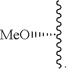

In certain embodiments, in the compound of Formula II, R¹ is

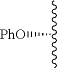

In certain embodiments, in the compound of Formula II, R¹ is

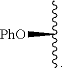

In certain embodiments, in the compound of Formula II, R¹ is C₀-C₆alkyl-cyano optionally substituted with 1, 2, 3, or 4 substituents.

In certain embodiments, in the compound of Formula II, R¹ is alkyl optionally substituted with 1, 2, 3, or 4 substituents.

In certain embodiments, in the compound of Formula II, R¹ is alkenyl optionally substituted with 1, 2, 3, or 4 substituents. In certain embodiments, in the compound of Formula II, R¹ is alkynyl optionally substituted with 1, 2, 3, or 4 substituents. In certain embodiments, in the compound of Formula II, R¹ is haloalkyl optionally substituted with 1, 2, 3, or 4 substituents. In certain embodiments, in the compound of Formula II, R¹ is F.

In certain embodiments, in the compound of Formula II, R¹ is Cl.

In certain embodiments, in the compound of Formula II, R¹ is Br.

In certain embodiments, in the compound of Formula II, R¹ is aryl optionally substituted with 1, 2, 3, or 4 substituents.

In certain embodiments, in the compound of Formula II, R¹ is arylalkyl optionally substituted with 1, 2, 3, or 4 substituents.

In certain embodiments, in the compound of Formula II, R¹ is heteroaryl optionally substituted with 1, 2, 3, or 4 substituents.

In certain embodiments, in the compound of Formula II, R¹ is heteroaryl alkyl optionally substituted with 1, 2, 3, or 4 substituents.

In certain embodiments, in the compound of Formula II, R¹ is heterocycle optionally substituted with 1, 2, 3, or 4 substituents.

In certain embodiments, in the compound of Formula II, R¹ is heterocycloalkyl optionally substituted with 1, 2, 3, or 4 substituents.

In certain embodiments, in the compound of Formula II, R¹ is haloalkoxy optionally substituted with 1, 2, 3, or 4 substituents.

In certain embodiments, in the compound of Formula II, R¹ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³ C₀-C₆alkyl-O—C(O)R³, C₀-C₆alkyl-O—S(O)R³, C₀-C₆alkyl-O—C(S)R³, —N═S(O)(R³)₂, C₀-C₆alkylN³, or C₀-C₆alkyl-O—S(O)₂R³, each of which is optionally substituted with 1, 2, 3, or 4 substituents.

In certain embodiments, in the compound of Formula II, R² is aryl optionally substituted with 1, 2, 3, or 4 substituents.

In certain embodiments, in the compound of Formula II, R² is heterocycle optionally substituted with 1, 2, 3, or 4 substituents.

In certain embodiments, in the compound of Formula II, R² is heteroaryl containing 1 or 2 heteroatoms independently selected from N, O, and S optionally substituted with 1, 2, 3, or 4 substituents.

In certain embodiments, in the compound of Formula II, R² is selected from

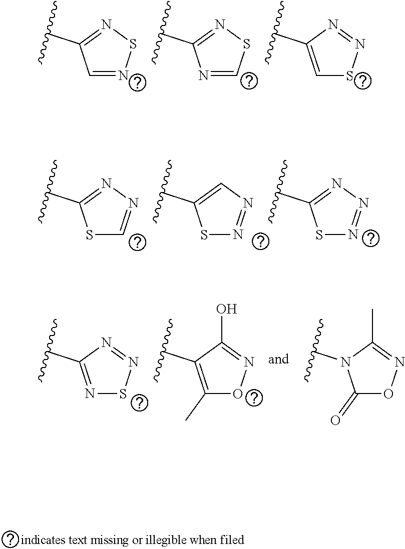

In certain embodiments, in the compound of Formula II, R² is heterocycle optionally substituted with 1, 2, 3, or 4 substituents.

In certain embodiments, in the compound of Formula II, R² is —NR⁸—S(O)—R³ optionally substituted with 1, 2, 3, or 4 substituents.

In certain embodiments, in the compound of Formula II, R² is —NR⁸—C(S)—R³ optionally substituted with 1, 2, 3, or 4 substituents.

In certain embodiments, in the compound of Formula II, R² is —NR⁸—S(O)(NR⁶)—R³ optionally substituted with 1, 2, 3, or 4 substituents.

In certain embodiments, in the compound of Formula II, R² is —N═S(O)(R³)₂ optionally substituted with 1, 2, 3, or 4 substituents.

In certain embodiments, in the compound of Formula II, R² is —NR⁸C(O)NR⁹S(O)₂R³ optionally substituted with 1, 2, 3, or 4 substituents.

In certain embodiments, in the compound of Formula II, R² is —NR⁸—S(O)₂—R¹⁰ optionally substituted with 1, 2, 3, or 4 substituents.

In certain embodiments, in the compound of Formula II, R² is —NR⁸—C(NR⁶)—R³ optionally substituted with 1, 2, 3, or 4 substituents.

In certain embodiments, in the compound of Formula II, R² is hydrogen.

In certain embodiments, in the compound of Formula II, R² is R¹⁰,

In certain embodiments, in the compound of Formula II, R² is alkyl-C(O)—R³.

In certain embodiments, in the compound of Formula II, R² is —C(O)—R³.

In certain embodiments, in the compound of Formula II, R² is alkyl.

In certain embodiments, in the compound of Formula II, R² is haloalkyl.

In certain embodiments, in the compound of Formula II, R² is —OC(O)R³.

In certain embodiments, in the compound of Formula II, R² is —NR⁸—C(O)R¹⁰.

In certain embodiments, in the compound of Formula II, R² is alkenyl optionally substituted with 1, 2, 3, or 4 substituents.

In certain embodiments, in the compound of Formula II, R² is allyl optionally substituted with 1, 2, 3, or 4 substituents.

In certain embodiments, in the compound of Formula II, R² is alkynyl optionally substituted with 1, 2, 3, or 4 substituents.

In certain embodiments, in the compound of Formula II, R² is —NR⁶-alkenyl optionally substituted with 1, 2, 3, or 4 substituents.

In certain embodiments, in the compound of Formula II, R² is —O-alkenyl optionally substituted with 1, 2, 3, or 4 substituents.

In certain embodiments, in the compound of Formula II, R² is —NR⁶-alkynyl optionally substituted with 1, 2, 3, or 4 substituents.

In certain embodiments, in the compound of Formula II, R² is —NR⁶-heteroaryl optionally substituted with 1, 2, 3, or 4 substituents.

In certain embodiments, in the compound of Formula II, R² is —NR⁶-aryl optionally substituted with 1, 2, 3, or 4 substituents.

In certain embodiments, in the compound of Formula II, R² is —O-heteroaryl optionally substituted with 1, 2, 3, or 4 substituents.

In certain embodiments, in the compound of Formula II, R² is —O-aryl optionally substituted with 1, 2, 3, or 4 substituents.

In certain embodiments, in the compound of Formula II, R² is —O-alkynyl optionally substituted with 1, 2, 3, or 4 substituents.

In certain embodiments, in the compound of Formula II, R² is selected from and

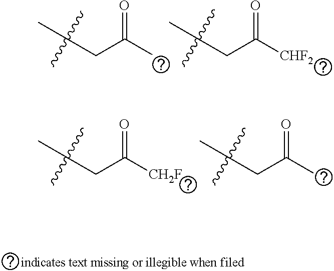

In certain embodiments, in the compound of Formula II, R² is selected from

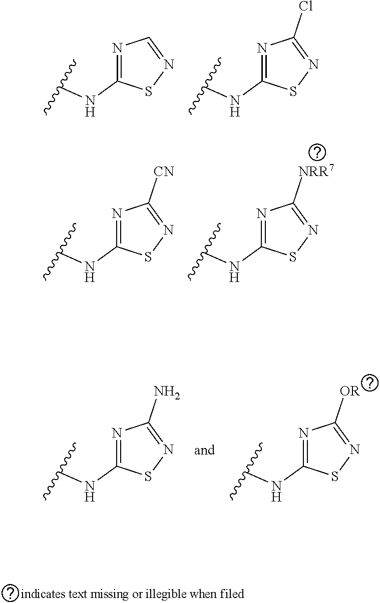

In certain embodiments, in the compound of Formula II, R² is selected from

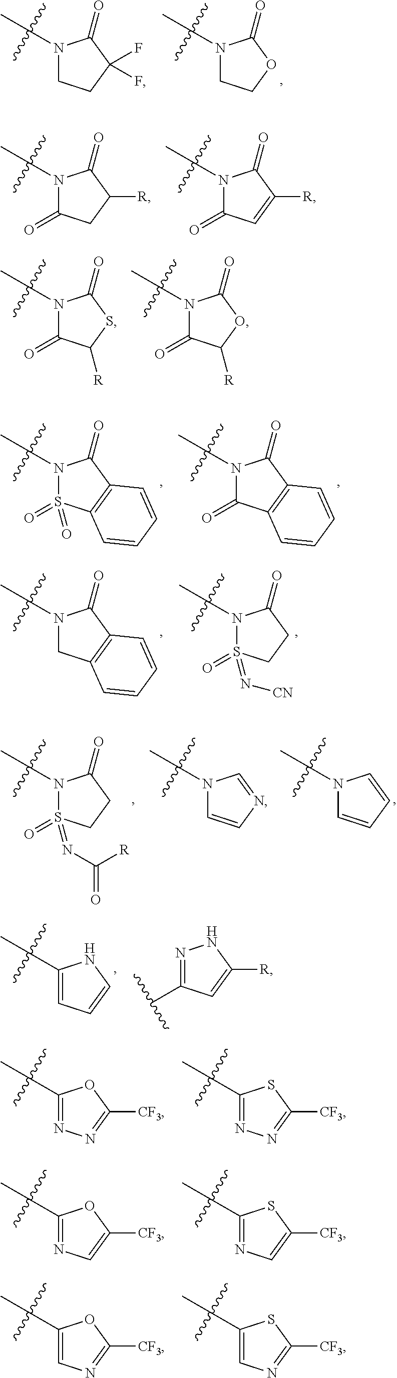

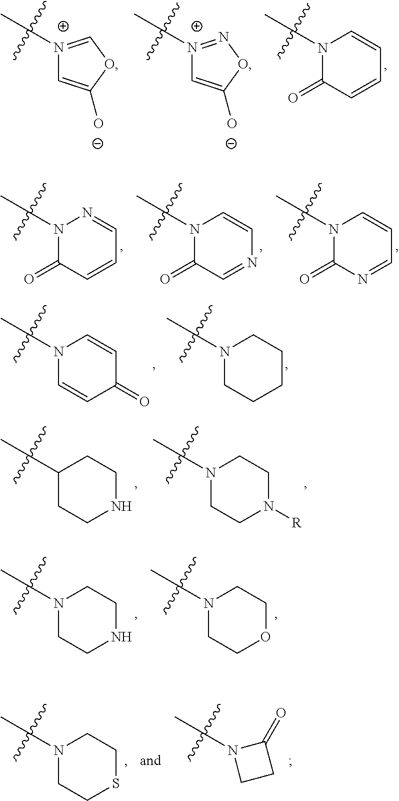

wherein R is an optional substituent as defined herein.

In certain embodiments, in the compound of Formula II, R² is selected from

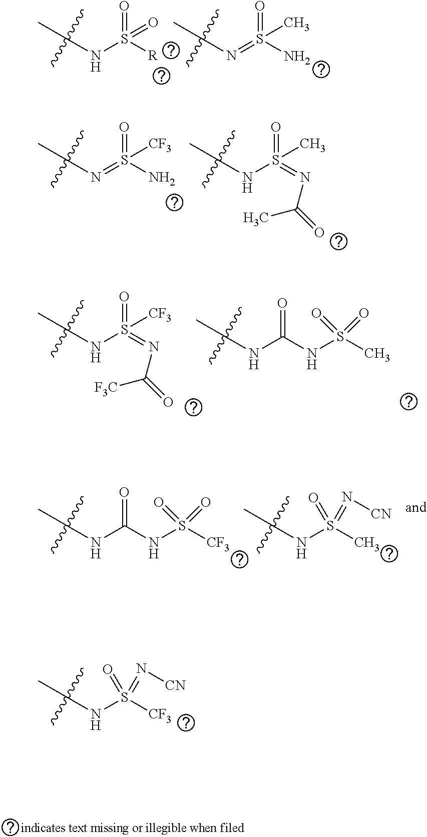

In certain embodiments, in the compound of Formula II, R^{2A} is selected from

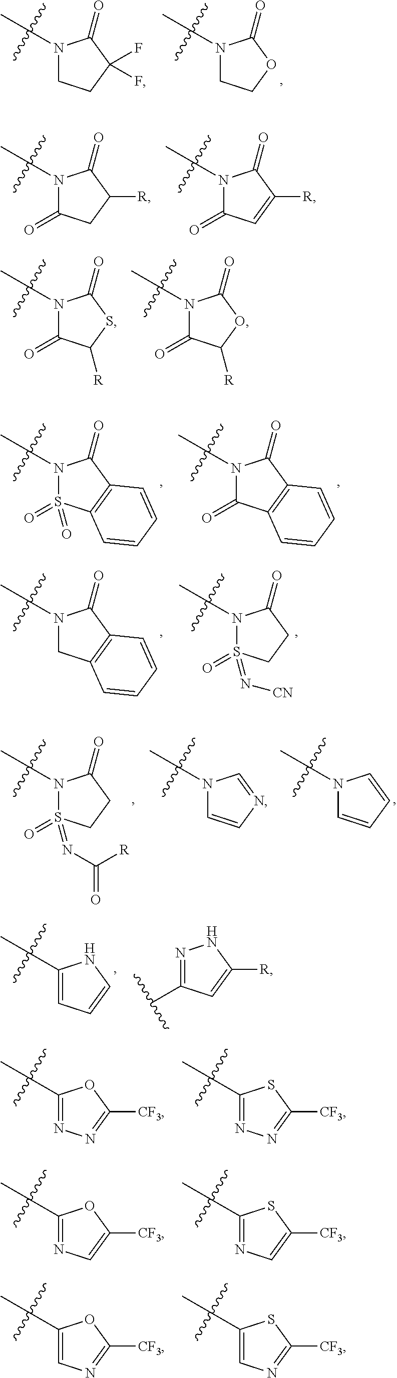

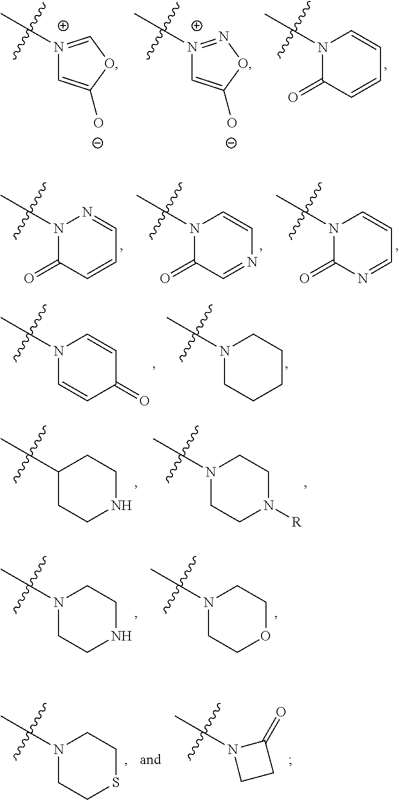

wherein R is an optional substituent as defined herein.

In certain embodiments, in the compound of Formula II, R^{2A} is selected from

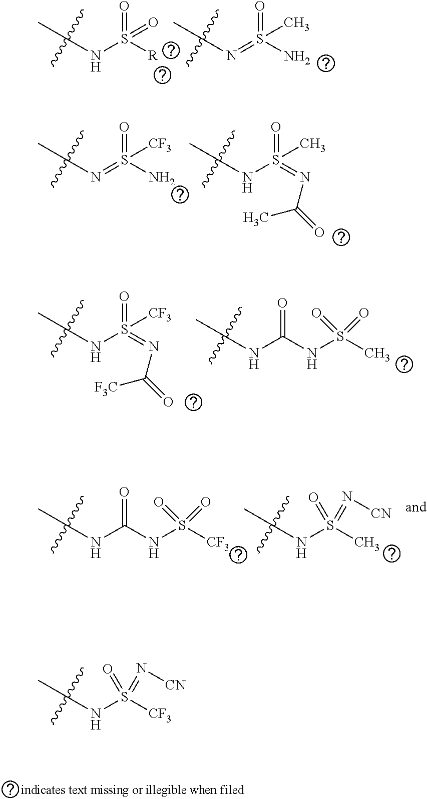

In certain embodiments, in the compound of Formula II, R² is selected from

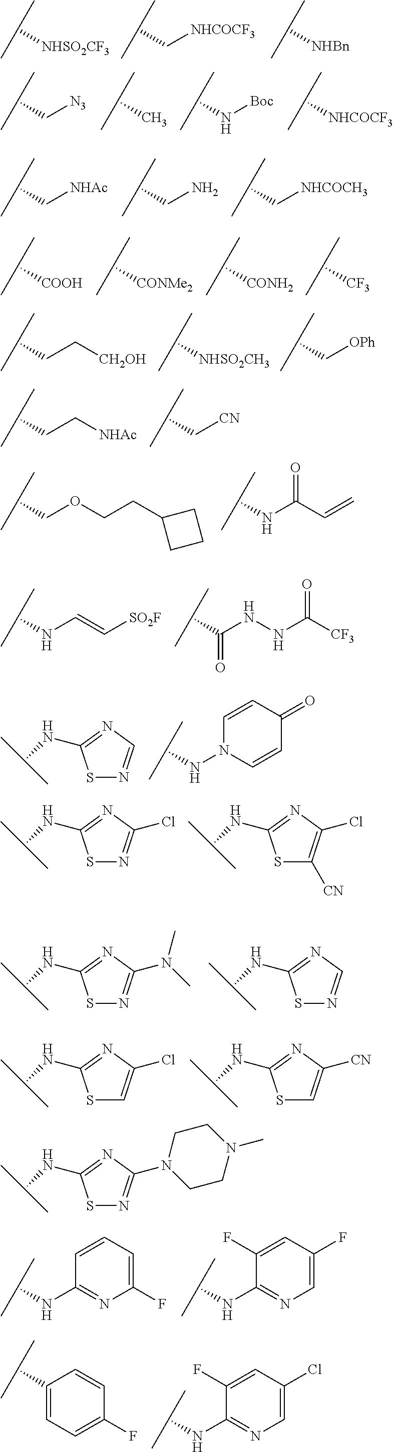

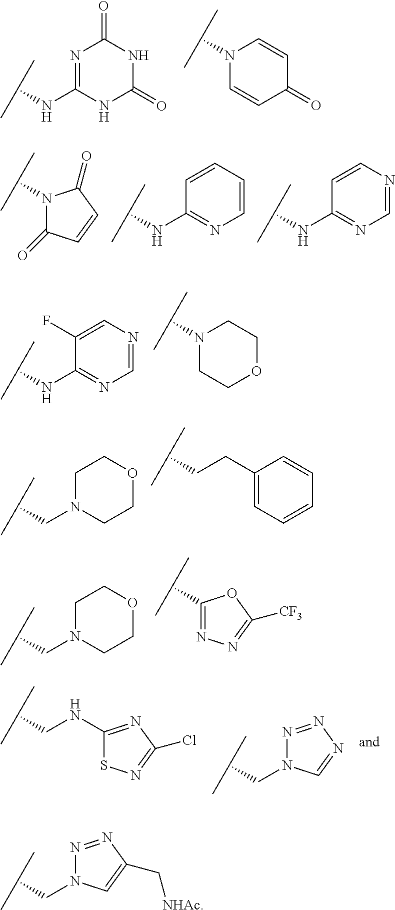

In certain embodiments, in the compound of Formula II, R² is selected from

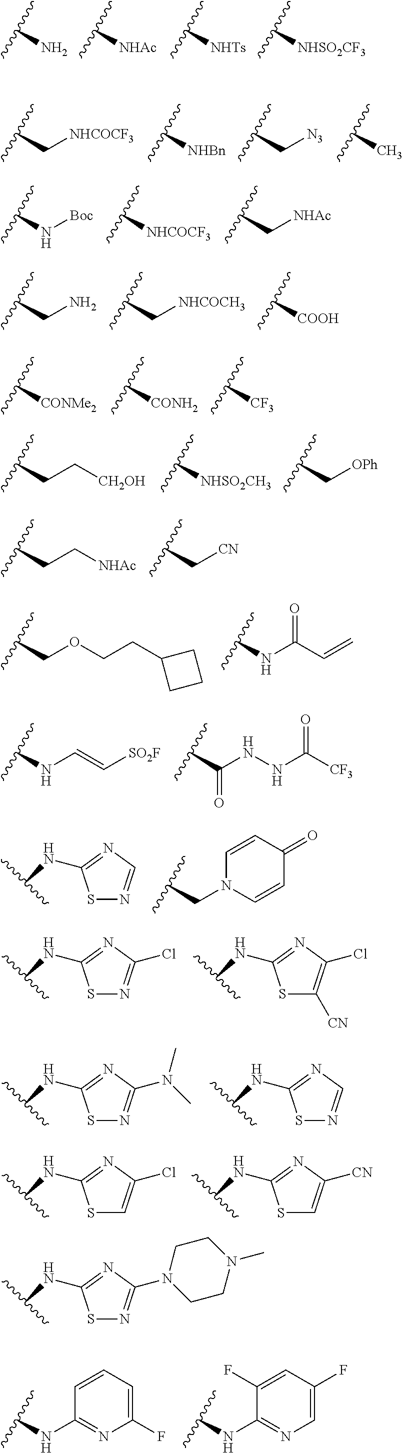

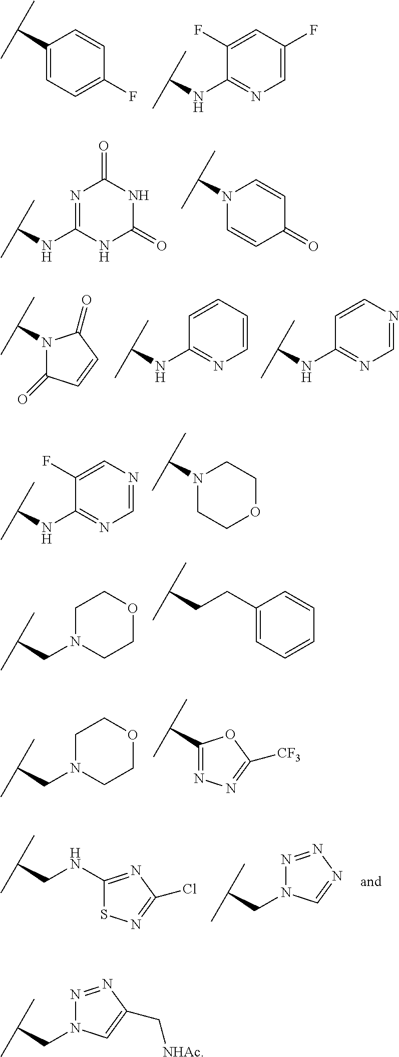

In certain embodiments, in the compound of Formula II, R² is selected from

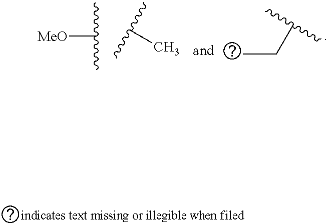

In certain embodiments, in the compound of Formula II, R² is selected from

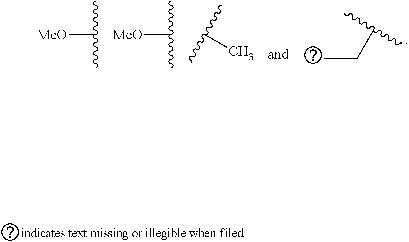

In certain embodiments, in the compound of Formula II, R² is selected from

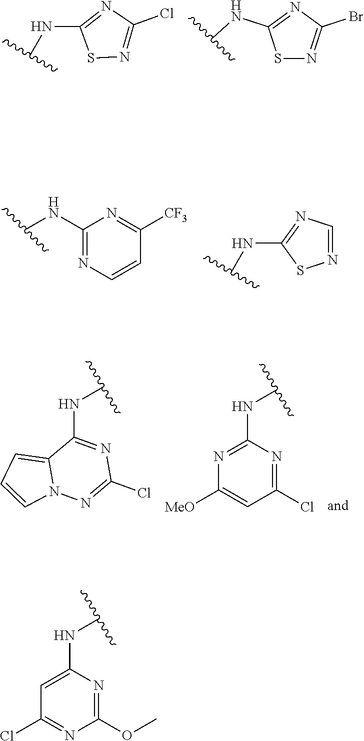

In certain embodiments, in the compound of Formula II, R² is selected from

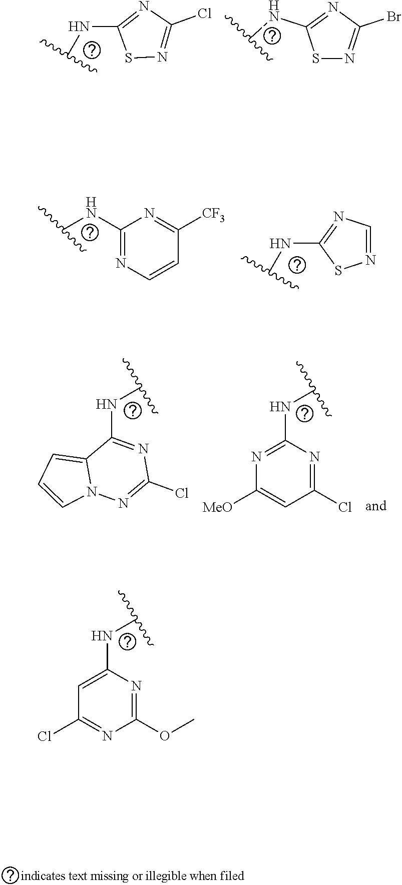

In certain embodiments, in the compound of Formula II, R² is selected from

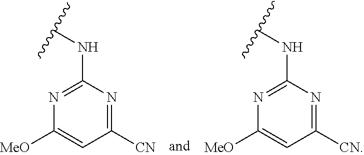 [SMILES: *Nc1nc(C#N)cc(OC)n1 ; *Nc1nc(C#N)cc(OC)n1]

In certain embodiments, in the compound of Formula II, R² is selected from

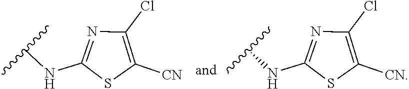

In certain embodiments, in the compound of Formula II, R² is selected from

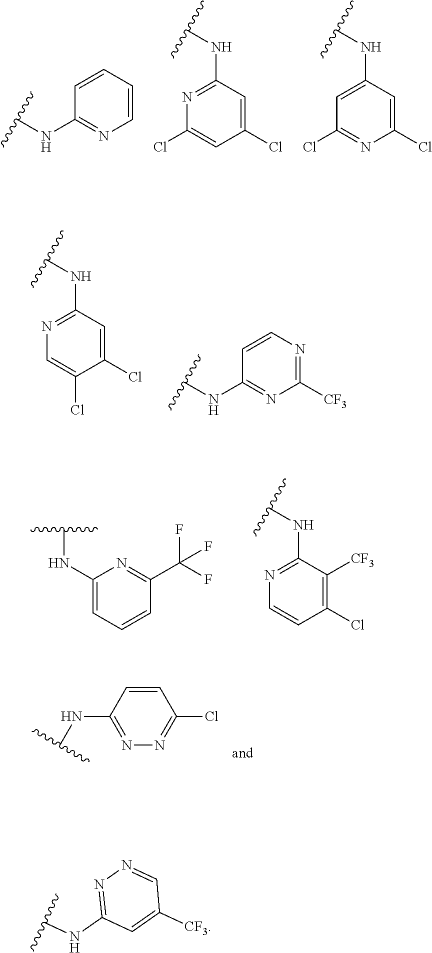

In certain embodiments, in the compound of Formula II, R² is selected from

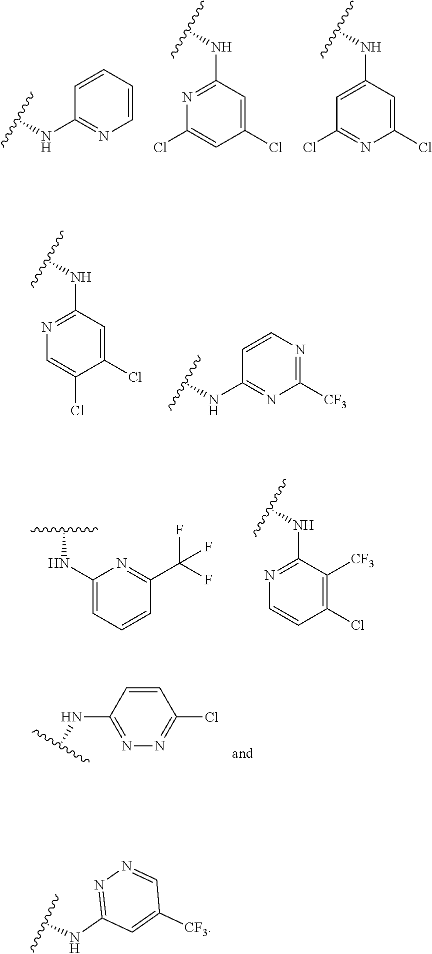

In certain embodiments, in the compound of Formula II, R² is selected from

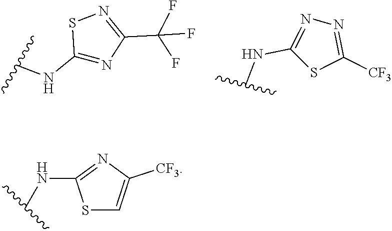 [SMILES: *Nc1nc(C(F)(F)F)ns1 ; *Nc1nnc(C(F)(F)F)s1 ; *Nc1nc(C(F)(F)F)cs1]

In certain embodiments, in the compound of Formula II, R² is selected from

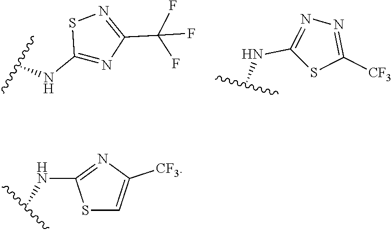

In certain embodiments, in the compound of Formula II, R² is selected from

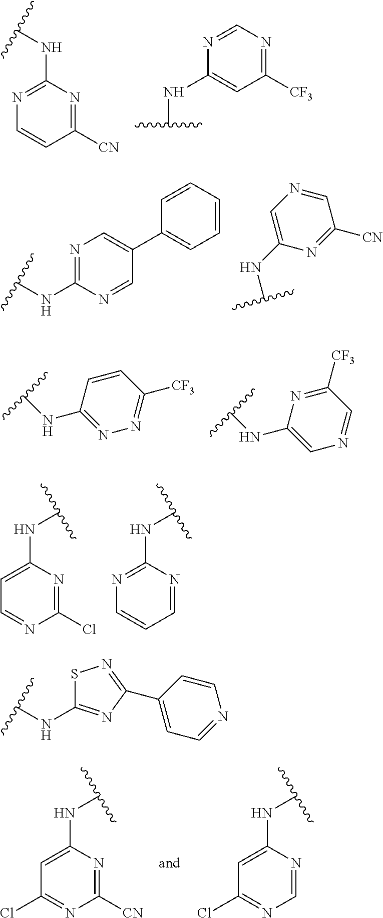

In certain embodiments, in the compound of Formula II, R² is selected from

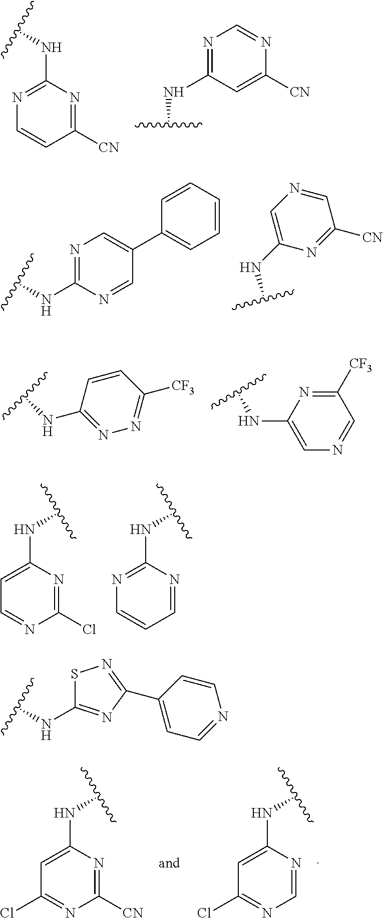

In certain embodiments, in the compound of Formula II, R² is selected from

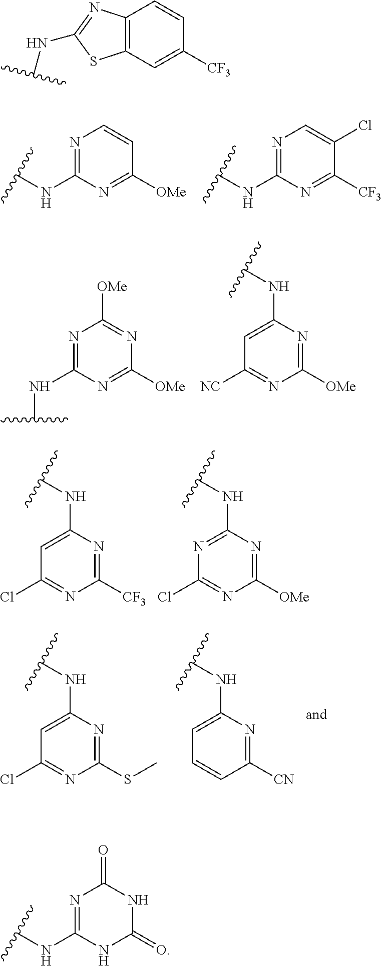

In certain embodiments, in the compound of Formula II, R² is selected from

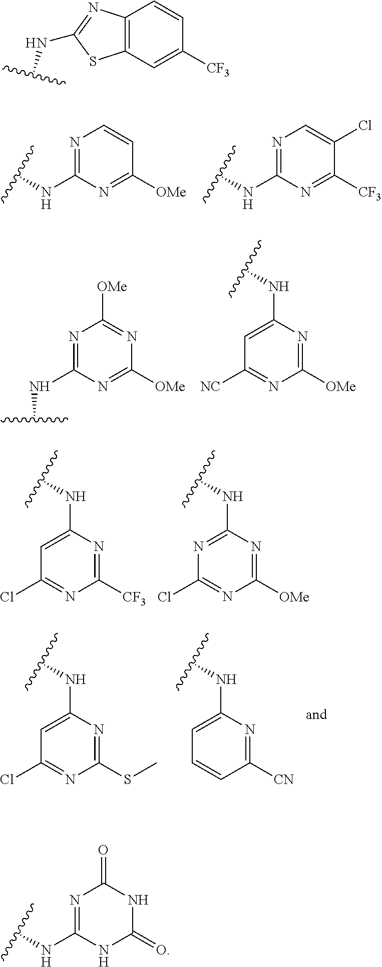

In certain embodiments, in the compound of Formula II, R² is selected from

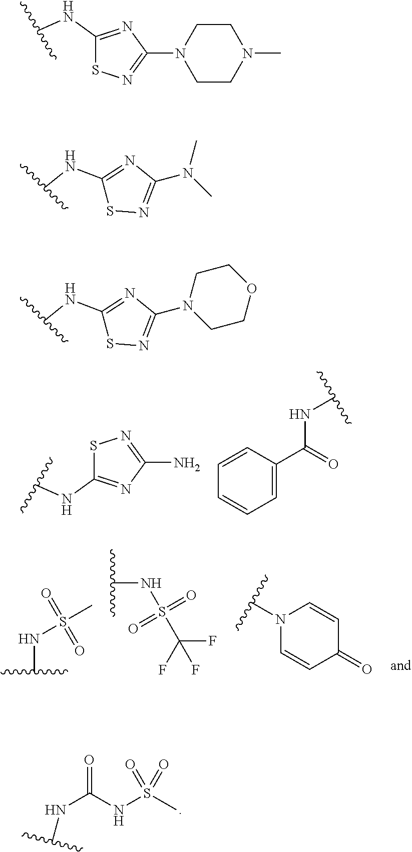

In certain embodiments, in the compound of Formula II, R² is selected from

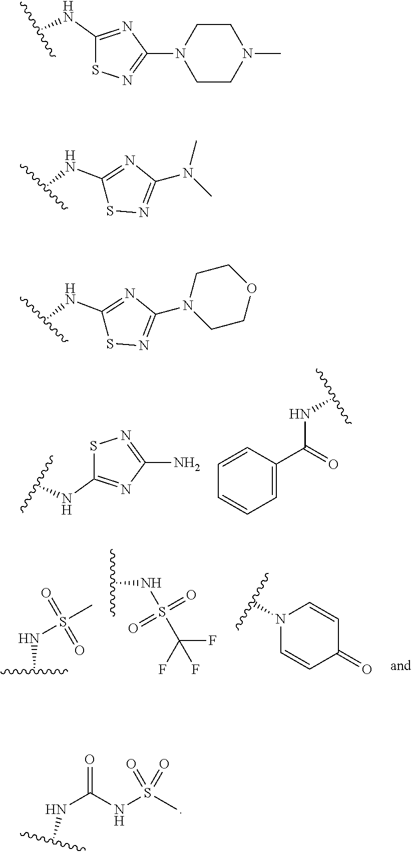

In certain embodiments, in the compound of Formula II, R² is selected from

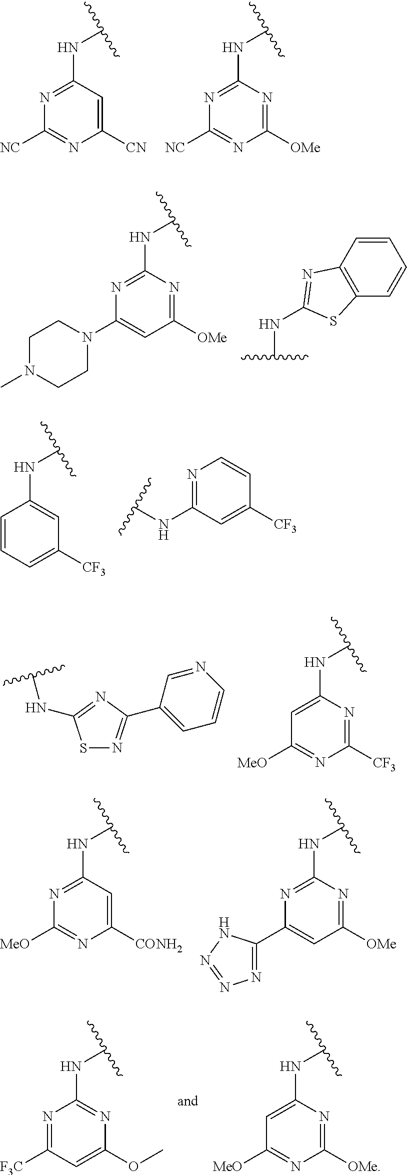

In certain embodiments, in the compound of Formula II, R² is selected from

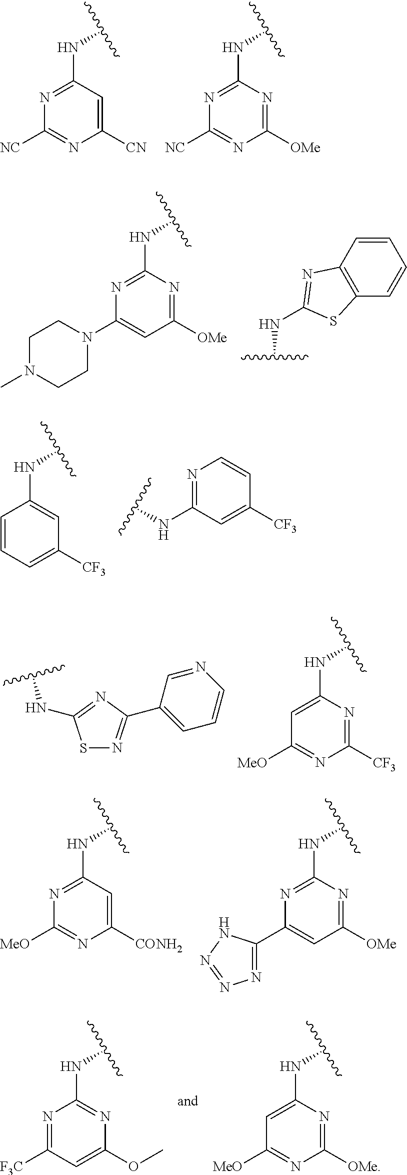

In certain embodiments, in the compound of Formula II, R² is selected from

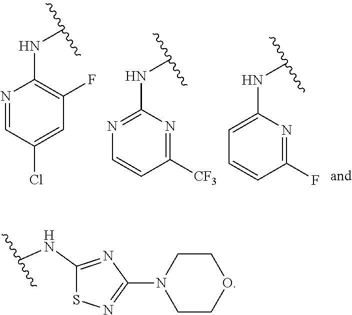

In certain embodiments, in the compound of Formula II, R² is selected from

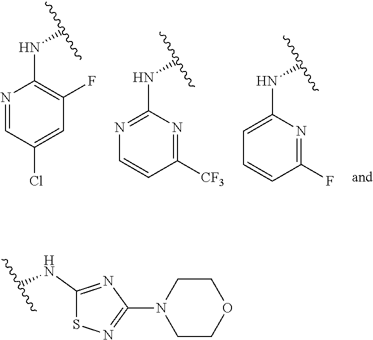

In certain embodiments, in the compound of Formula II, R² is selected from

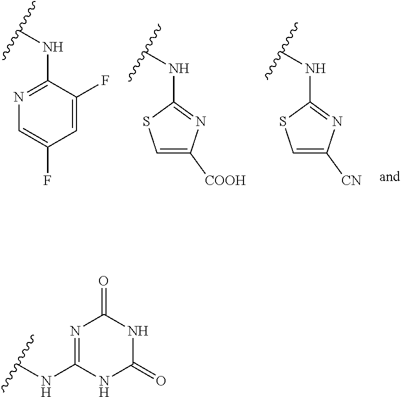 [SMILES: *Nc1ncc(F)cc1F ; *Nc1nc(C(=O)O)cs1 ; *Nc1nc(C#N)cs1 ; *Nc1nc(=O)[nH]c(=O)[nH]1]

In certain embodiments, in the compound of Formula II, R² is selected from

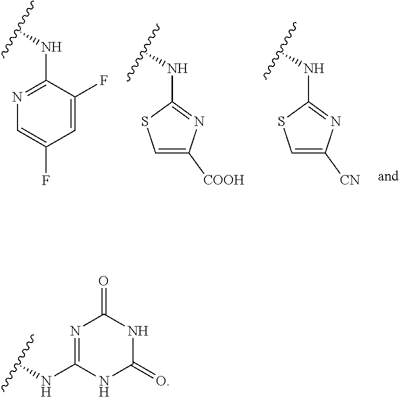

In certain embodiments, in the compound of Formula II, R² is selected from

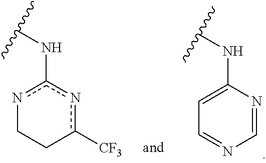

In certain embodiments, in the compound of Formula II, R² is selected from

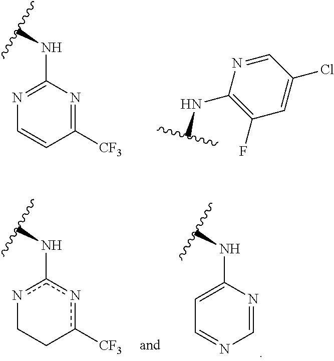

In certain embodiments, in the compound of Formula II, R² is selected from

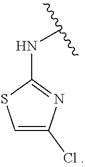 [SMILES: *Nc1nc(Cl)cs1]

In certain embodiments, in the compound of Formula II, R² is selected from

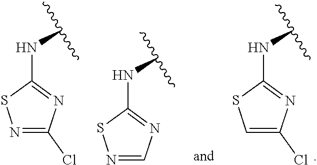

In certain embodiments, in the compound of Formula II, R² is selected from

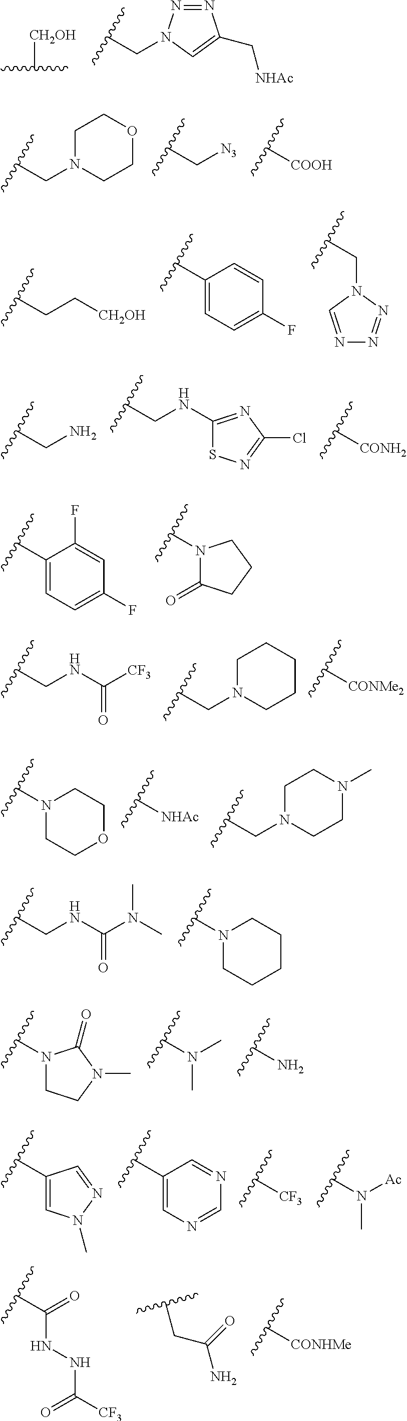

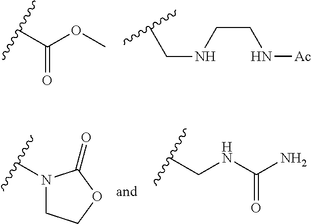

In certain embodiments, in the compound of Formula II, R² or R^{2A} is selected from

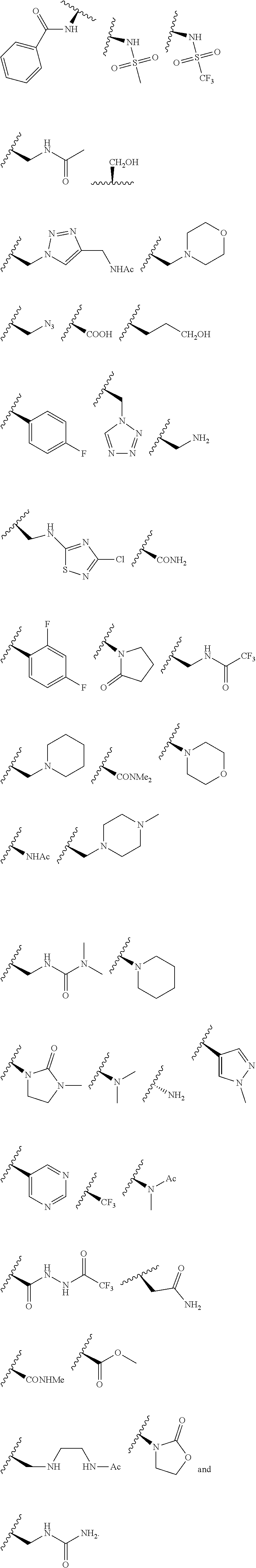

In certain embodiments, in the compound of Formula II, R² is selected from

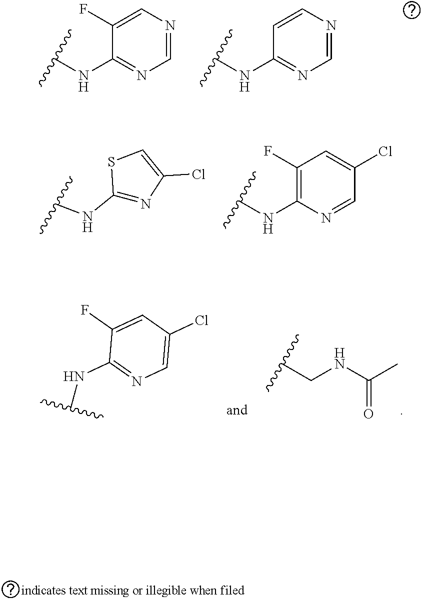

In certain embodiments, in the compound of Formula II, R² is selected from

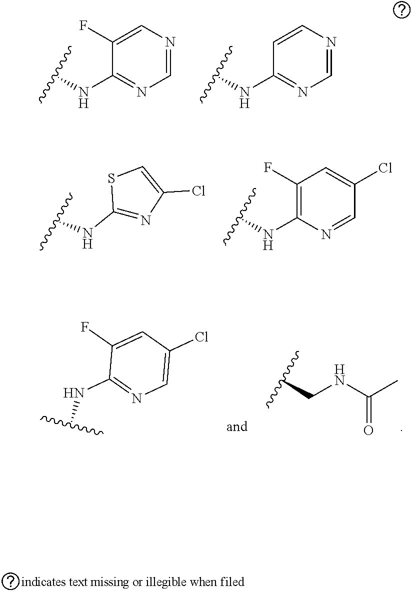

In certain embodiments, in the compound of Formula II, R² is selected from

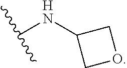

In certain embodiments, in the compound of Formula II, R² is selected from

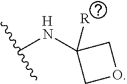

In certain embodiments, in the compound of Formula II, R² is selected from

In certain embodiments, in the compound of Formula II, R² is a spirocyclic heterocycle, for example, and without limitation,

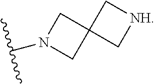 [SMILES: *N1CC2(CNC2)C1]

In certain embodiments, in the compound of Formula II, R² is a silicon containing heterocycle, for example, and without limitation,

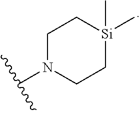 [SMILES: *N1CC[Si](C)(C)CC1]

In certain embodiments, in the compound of Formula II, R² is substituted with SF₅, for example, and without limitation,

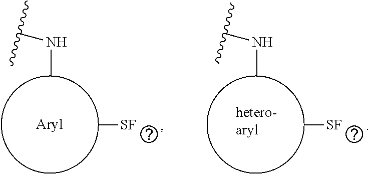

In certain embodiments, in the compound of Formula II, R² is substituted with a sulfoxime, for example, and without limitation,

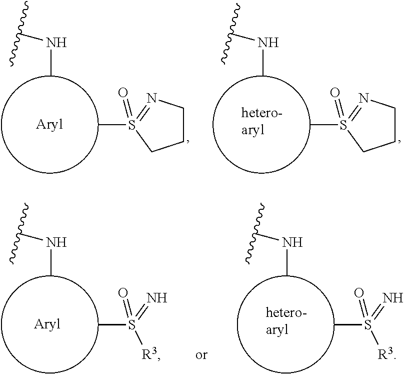

In certain embodiments, in the compound of Formula II, R¹⁰ is selected from bicyclic heterocycle.

In certain embodiments, in the compound of Formula II, R¹⁰ is selected from spirocyclic heterocycle.

In certain embodiments, in the compound of Formula II, R¹⁰ is selected from —NR⁶-heterocycle.

In certain embodiments, in the compound of Formula II, R¹⁰ is selected from

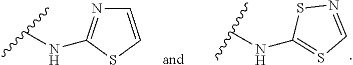

In certain embodiments, in the compound of Formula II, R¹⁰ is selected from

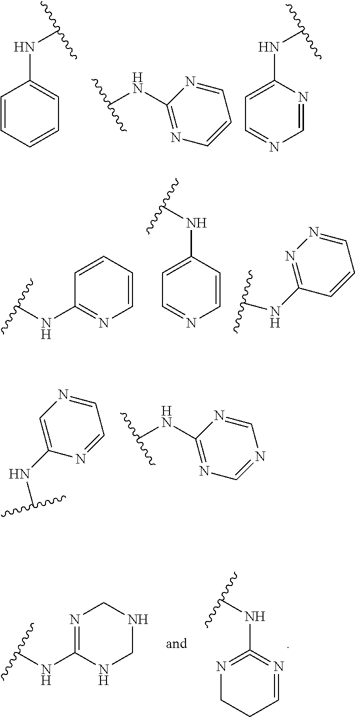

In certain embodiments, in the compound of Formula II, R¹⁰ is selected from

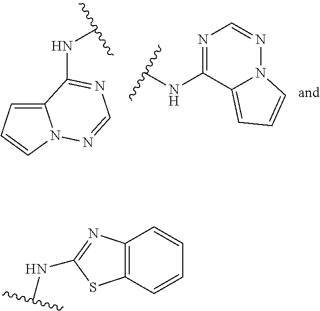 [SMILES: *Nc1ncnn2cccc12 ; *Nc1ncnn2cccc12 ; *Nc1nc2ccccc2s1]

In certain embodiments, in the compound of Formula II, R¹⁰ is selected from

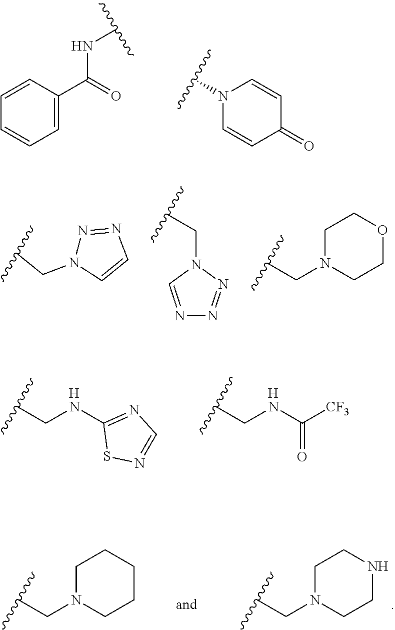

In certain embodiments, in the compound of Formula II, Cycle is selected from

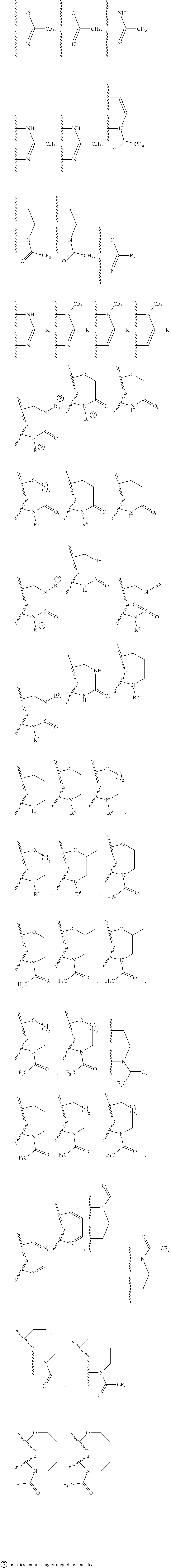

In certain embodiments, in the compound of Formula II, R³⁰ is selected from:

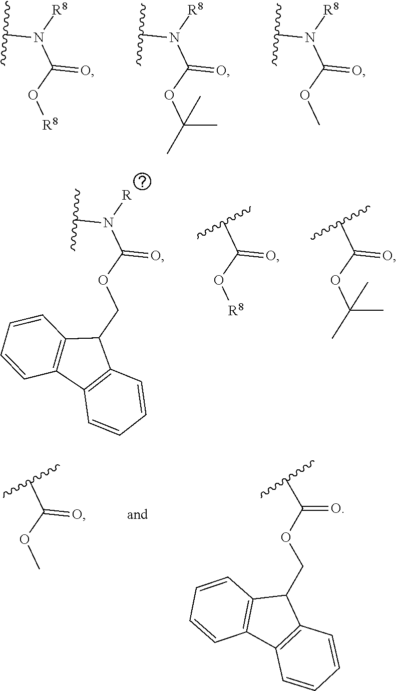

In certain embodiments, in the compound of Formula II, R²⁰⁰ is

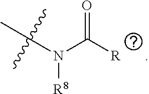

In certain embodiments, in the compound of Formula II, R²⁰⁰ is

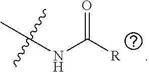

In certain embodiments, in the compound of Formula II, R²⁰⁰ is

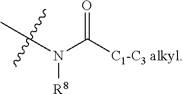

In certain embodiments, in the compound of Formula II, R²⁰⁰ is

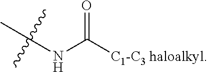

In certain embodiments, in the compound of Formula II, R²⁰⁰ is

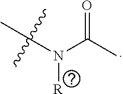

In certain embodiments, in the compound of Formula II, R²⁰⁰ is

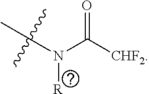

In certain embodiments, in the compound of Formula II, R²⁰⁰ is

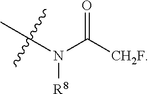

In certain embodiments, in the compound of Formula II, R²⁰⁰ is

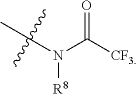

In certain embodiments, in the compound of Formula II, R²⁰⁰ is

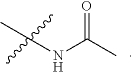 [SMILES: *NC(C)=O]

In certain embodiments, in the compound of Formula II, R²⁰⁰ is

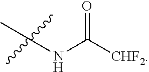 [SMILES: *NC(=O)C(F)F]

In certain embodiments, in the compound of Formula II, R²⁰⁰ is

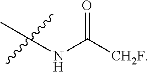

In certain embodiments, in the compound of Formula II, R²⁰⁰ is

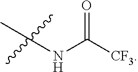 [SMILES: *NC(=O)C(F)(F)F]

Linkers

In non-limiting embodiments, in the compound of Formula II, Linker^{A} and Linker^{B} are independently selected from:

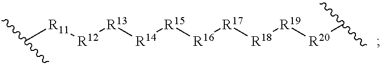

wherein:

R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, and, heterocycle, heteroaryl, —CH₂CH₂—[O—(CH₂)₂]ₙ—O—, CH₂CH₂—[O—(CH₂)₂]ₙ—NR⁶—, —CH₂CH₂—[O—(CH₂)₂]ₙ—, —[—(CH₂)₂—O—]ₙ—, [O—(CH₂)₂]ₙ—, —[O—CH(CH₃)C(O)]ₙ—, —[C(O)—CH(CH₃)—O]ₙ—,

[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;

n is independently selected at each instance from 0, 1, 2, 3, 4, 5, 6, 7, 8, 9, or 10;

R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, and, heteroaryl, and heterocycle;

and the remaining variables are as defined herein.

In one embodiment, in the compound of Formula II, Linker^{A} is bond and Linker^{B} is

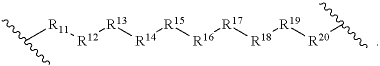

In one embodiment, in the compound of Formula II, Linker^{B} is bond and Linker^{A} is

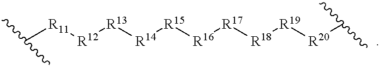

In one embodiment, in the compound of Formula II, a divalent residue of an amino acid is selected from

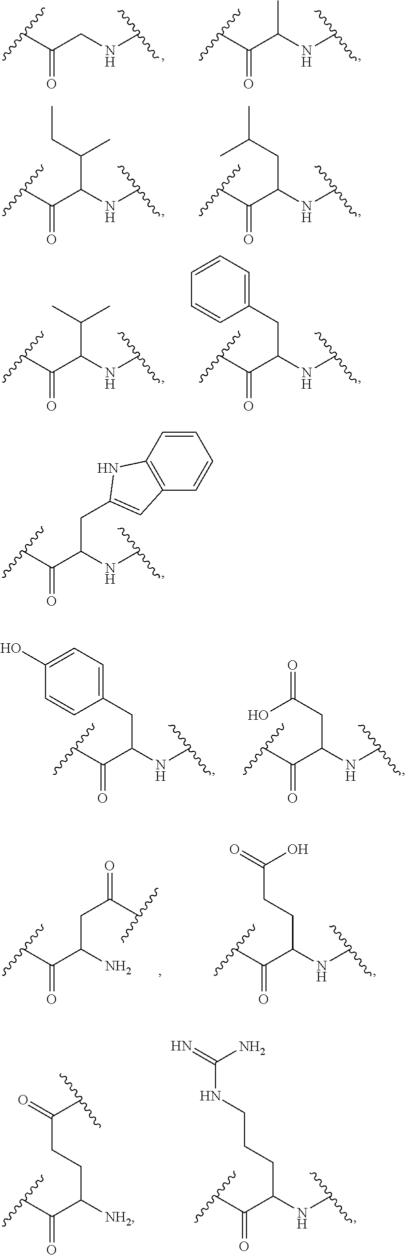

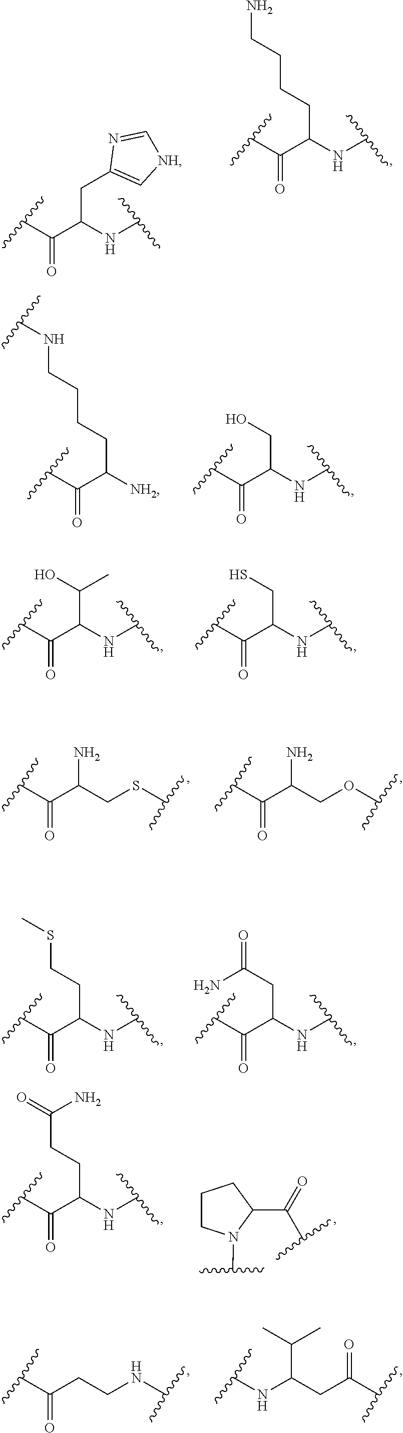

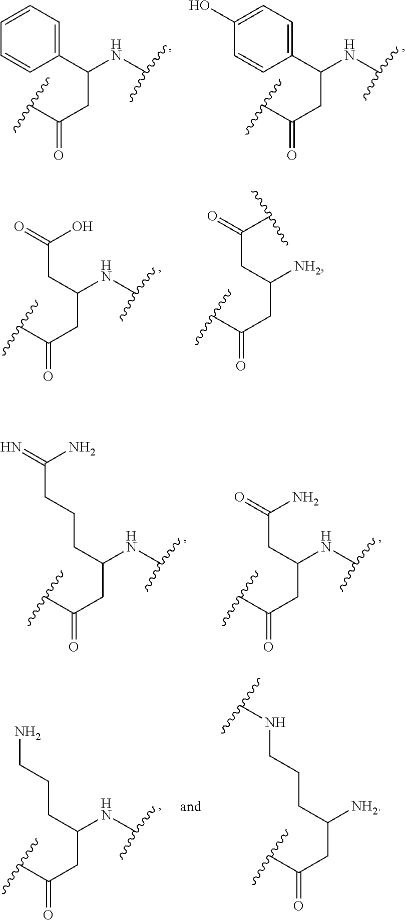

wherein the amino acid can be oriented in either direction and wherein the amino acid can be in the L- or D-form or a mixture thereof.

In one embodiment, in the compound of Formula II, a divalent residue of a dicarboxylic acid is generated from a nucleophilic addition reaction:

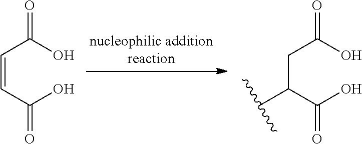 [SMILES: O=C(O)/C=C\C(=O)O ; *C(CC(=O)O)C(=O)O]

Non-limiting embodiments of a divalent residue of a dicarboxylic acid generated from a nucleophilic addition reaction include:

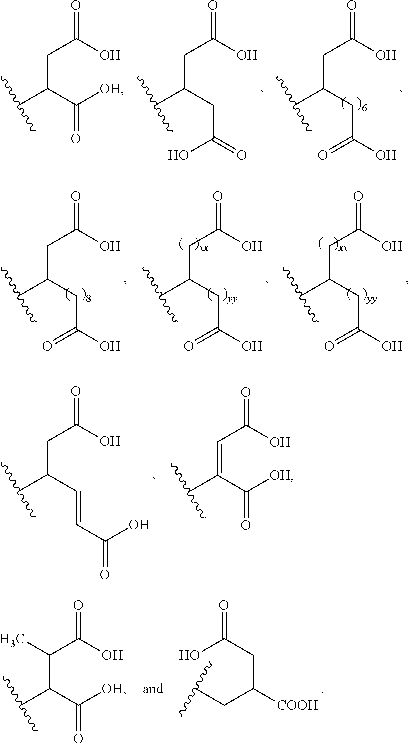

In one embodiment, in the compound of Formula II, a divalent residue of a dicarboxylic acid is generated from a condensation reaction:

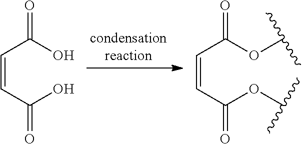 [SMILES: O=C(O)/C=C\C(=O)O ; *OC(=O)/C=C\C(=O)O*]

Non-limiting embodiments of a divalent residue of a dicarboxylic acid generated from a condensation include:

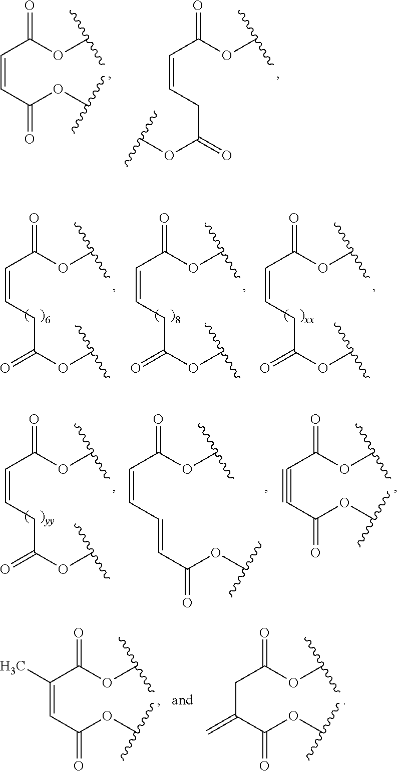

Non-limiting embodiments of a divalent residue of a saturated dicarboxylic acid include:

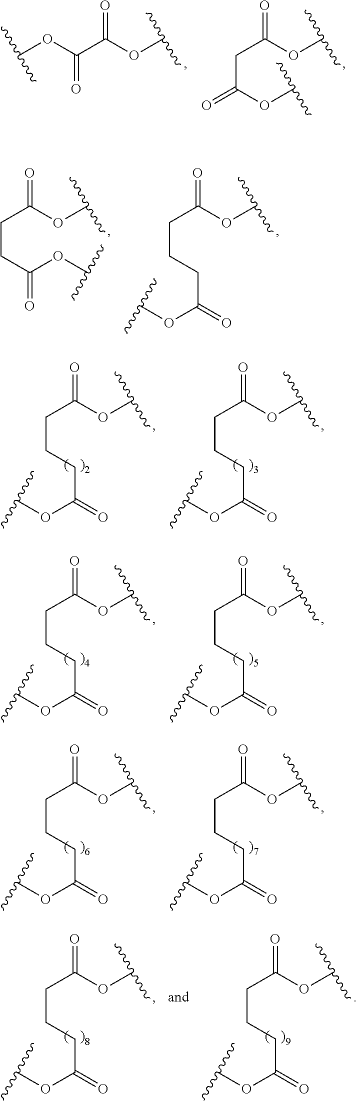

Non-limiting embodiments of a divalent residue of a saturated dicarboxylic acid include:

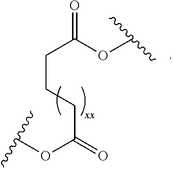

Non-limiting embodiments of a divalent residue of a saturated monocarboxylic acid is selected from butyric acid (—OC(O)(CH₂)₂CH₂—), caproic acid (—OC(O)(CH₂)₄CH₂—), caprylic acid (—OC(O)(CH₂)₅CH₂—), capric acid (—OC(O)(CH₂)₈CH₂—), lauric acid (—OC(O)(CH₂)₁₀CH₂—), myristic acid (—OC(O)(CH₂)₁₂CH₂—), pentadecanoic acid (—OC(O)(CH₂)₁₃CH₂—), palmitic acid (—OC(O)(CH₂)₁₄CH₂—), stearic acid (—OC(O)(CH₂)₁₆CH₂—), behenic acid (—OC(O)(CH₂)₂₀CH₂—), and lignoceric acid (—OC(O)(CH₂)₂₂CH₂—);

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

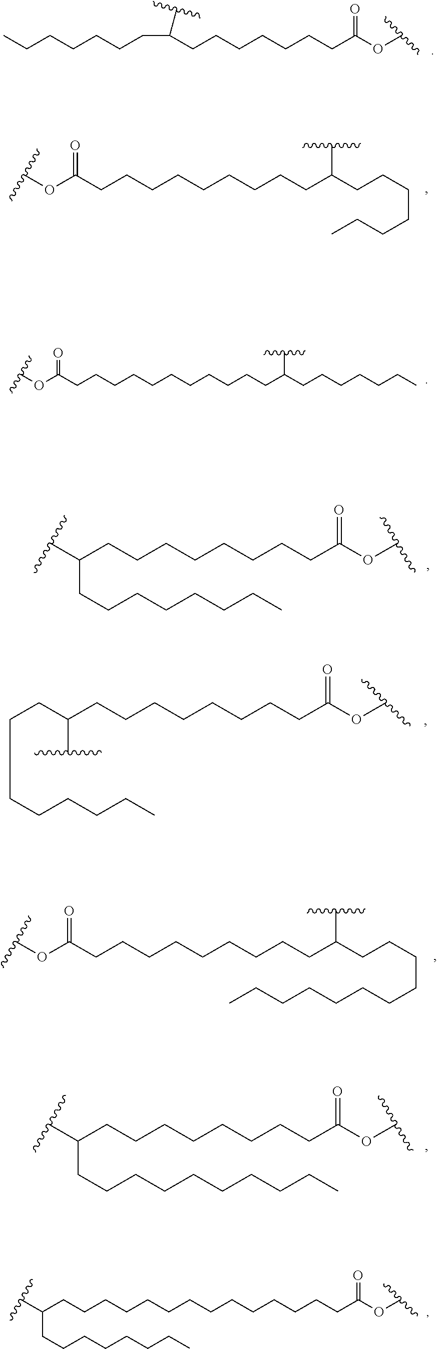

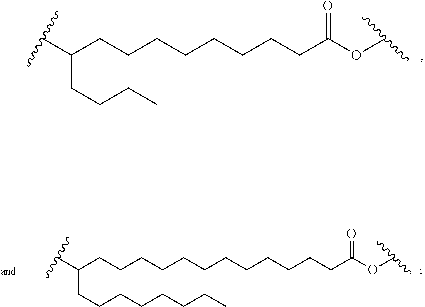 [SMILES: *OC(=O)CCCCCCCCC(*)CCCC ; *OC(=O)CCCCCCCCCCCCC(*)CCCCCCCC]

Non-limiting embodiments of a divalent residue of a fatty acid is selected from linoleic acid (—C(O)(CH₂)₇(CH)₂CH₂(CH)₂(CH₂)₄CH₂—), docosahexaenoic acid

(—C(O)(CH₂)₂(CHCHCH₂)₆CH₂—), eicosapentaenoic acid (—C(O)(CH₂)₃(CHCHCH₂)₅CH₂—), alpha-linolenic acid (—C(O)(CH₂)₇(CHCHCH₂)₃CH₂—) stearidonic acid

(—C(O)(CH₂)₄(CHCHCH₂)₄CH₂—), y-linolenic acid (—C(O)(CH₂)₄(CHCHCH₂)₃(CH₂)₃CH₂—), arachidonic acid (—C(O)(CH₂)₃(CHCHCH₂)₄(CH₂)₄CH₂—), docosatetraenoic acid (—C(O)(CH₂)₅(CHCHCH₂)₄(CH₂)₄CH₂—), palmitoleic acid (—C(O)(CH₂)₇CHCH(CH₂)₅CH₂—), vaccenic acid (—C(O)(CH₂)₉CHCH(CH₂)₅CH₂—), paullinic acid

(—C(O)(CH₂)₁₁CHCH(CH₂)₅CH₂—), oleic acid (—C(O)(CH₂)₇CHCH(CH₂)₇CH₂—), elaidic acid

(—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₃CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments, in the compound of Formula II, Linker^{C} is selected from:

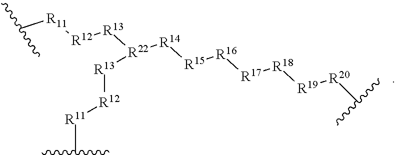

wherein:

R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;

and the remaining variables are as defined herein.

In certain embodiments, in the compound of Formula II, Linker^{D} is selected from:

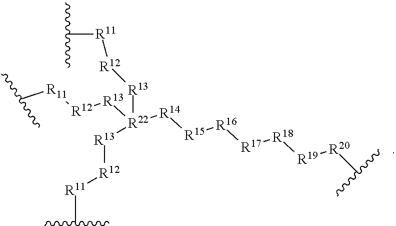

wherein:

R³² is independently at each occurrence selected from the group consisting of alkyl, N⁺X—, —C—, alkenyl, haloalkyl, aryl, heterocycle, and heteroaryl, each of which is optionally substituted with 1, 2, 3, or 4 substituents independently selected from R²¹;

X— is an anionic group, for example Br— or Cl⁻; and

all other variables are as defined herein.

In certain embodiments, in the compound of Formula II, Linker^{A} is selected from:

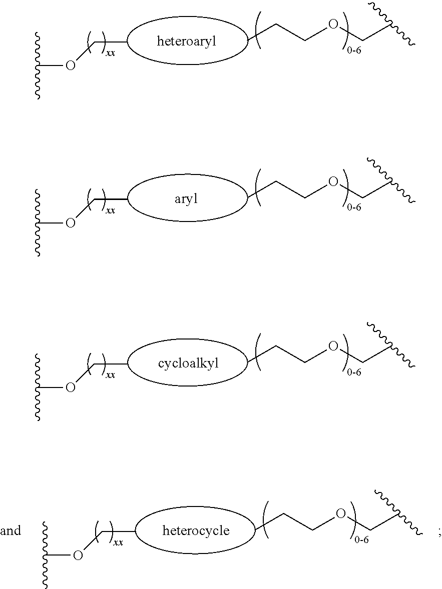

wherein each heteroaryl, heterocycle, cycloalkyl, and aryl can optionally be substituted with 1, 2, 3, or 4 of any combination of halogen, alkyl, haloalkyl, and, heteroaryl, heterocycle, or cycloalkyl, as allowed by valence.

In certain embodiments, in the compound of Formula II, Linker^{A} is selected from:

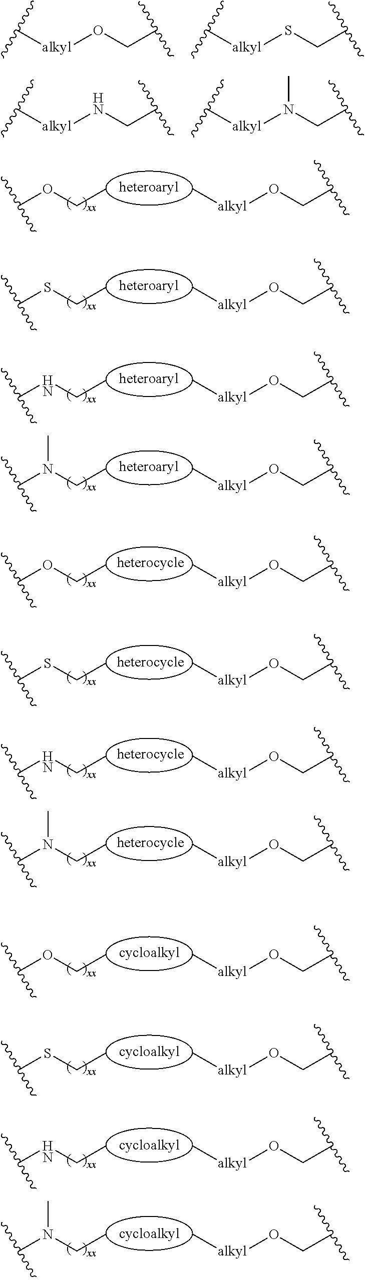

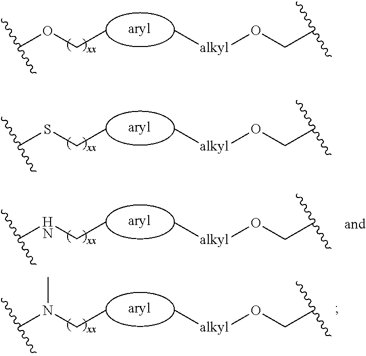

wherein each heteroaryl, heterocycle, cycloalkyl, and and can optionally be substituted with 1, 2, 3, or 4 of any combination of halogen, alkyl, haloalkyl, aryl, heteroaryl, heterocycle, or cycloalkyl, as allowed by valence.

In certain embodiments, in the compound of Formula II, Linker^{B} is selected from:

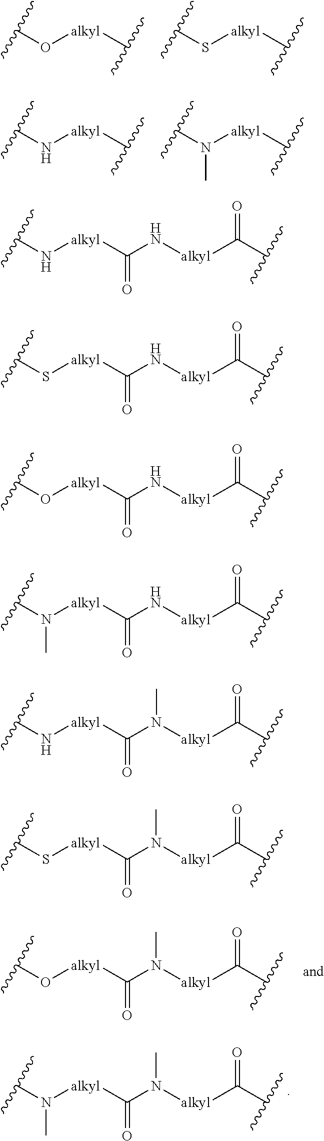

In certain embodiments, in the compound of Formula II, Linker^{B} is selected from:

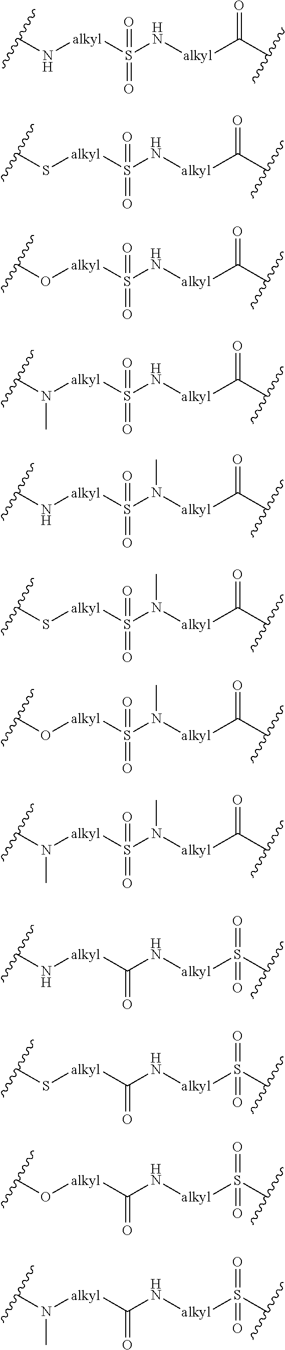

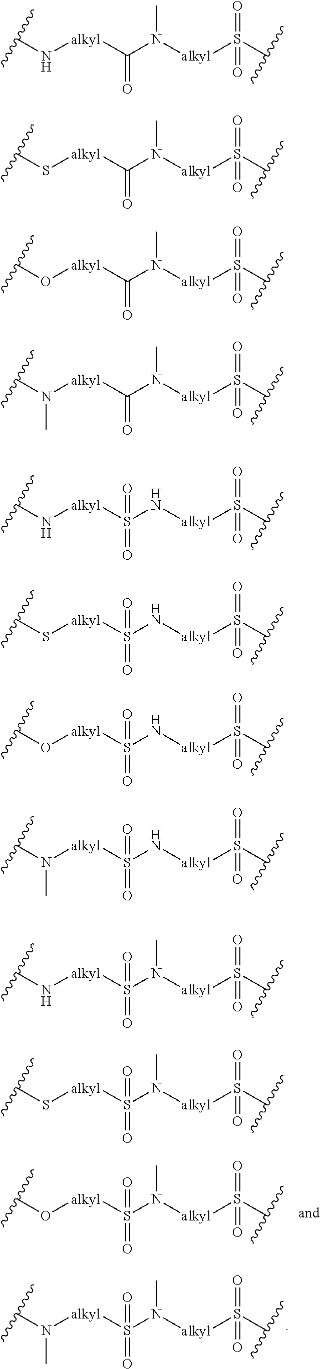

In certain embodiments, in the compound of Formula II, Linker^{B}, Linker^{C}, or Linker^{D} is selected from:

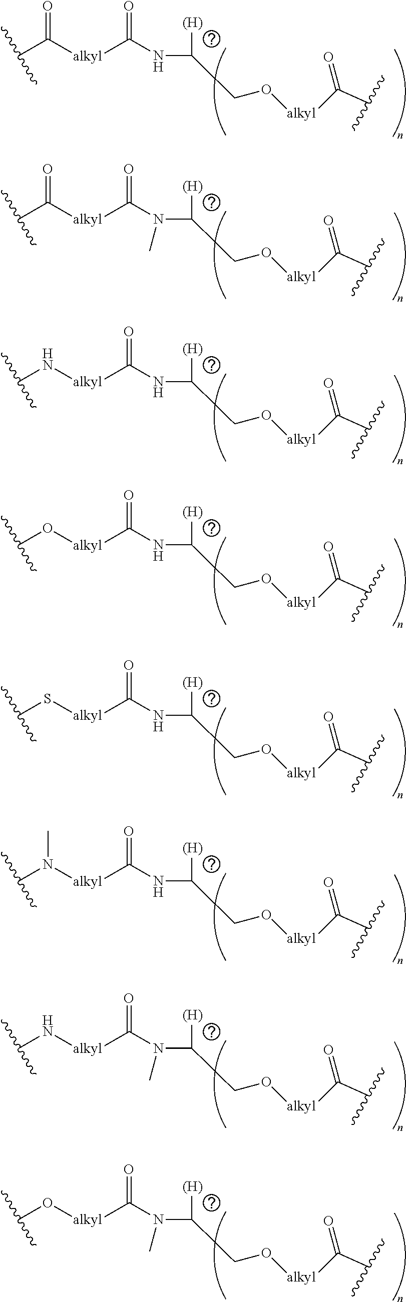

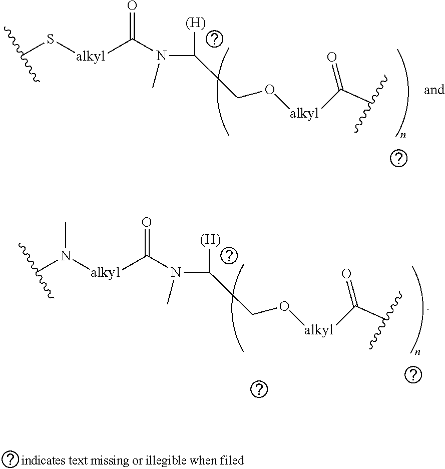

wherein tt is independently selected from 1, 2, or 3 and ss is 3 minus tt (3-tt).

In certain embodiments, in the compound of Formula II, Linker^{B}, Linker^{C}, or Linker^{D} is selected from:

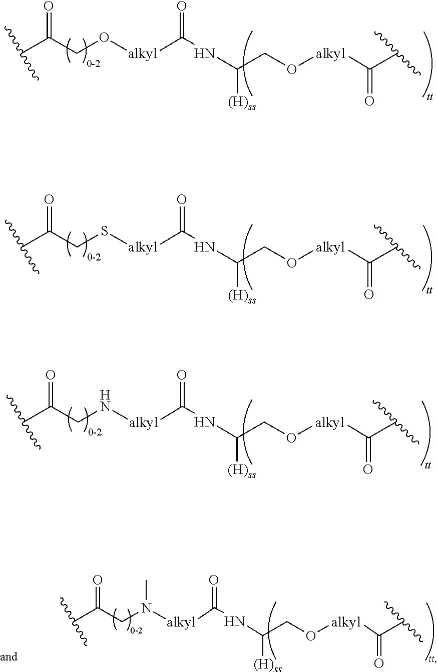

wherein tt and ss are as defined herein.

In certain embodiments, in the compound of Formula II, Linker^{B}, Linker^{C}, or Linker^{D} is selected from:

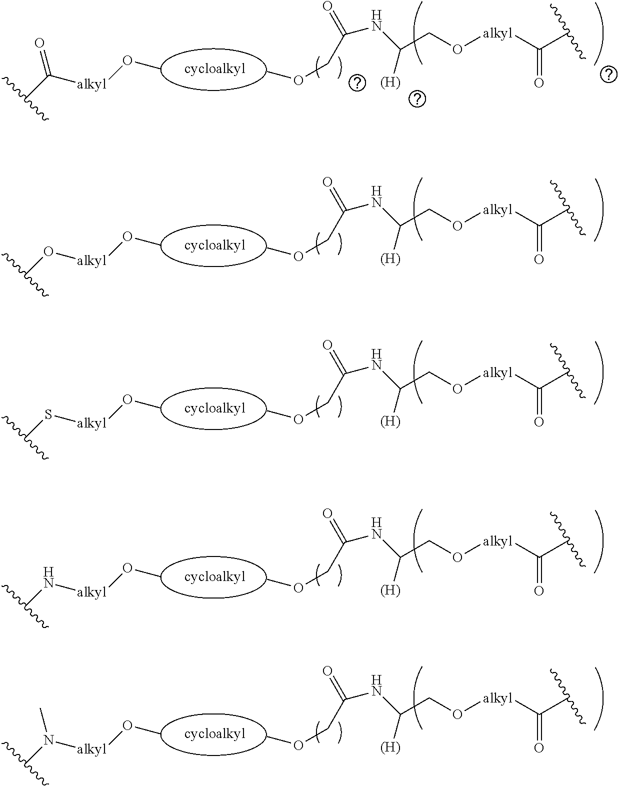

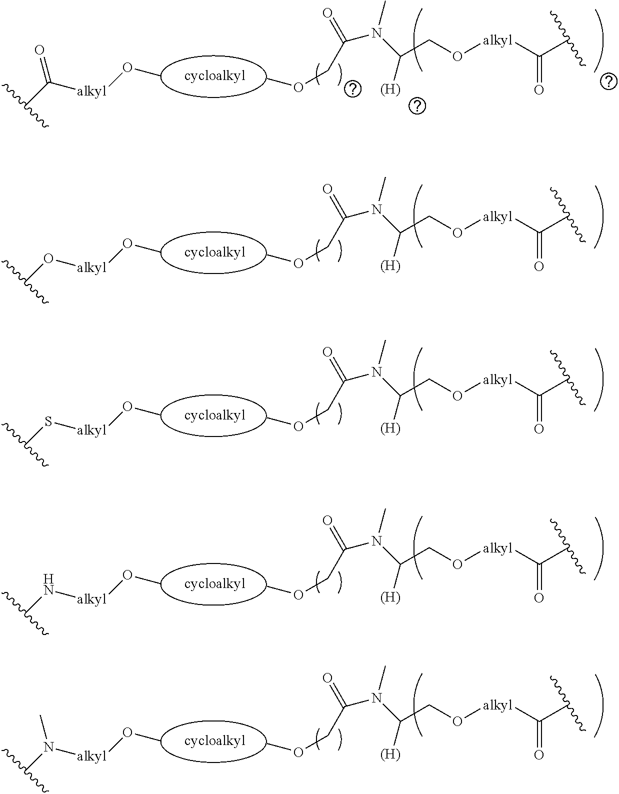

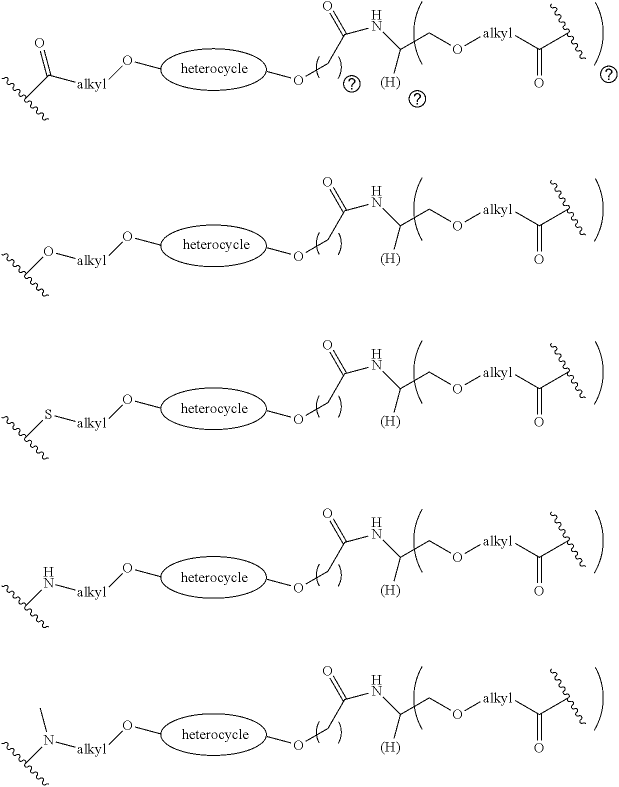

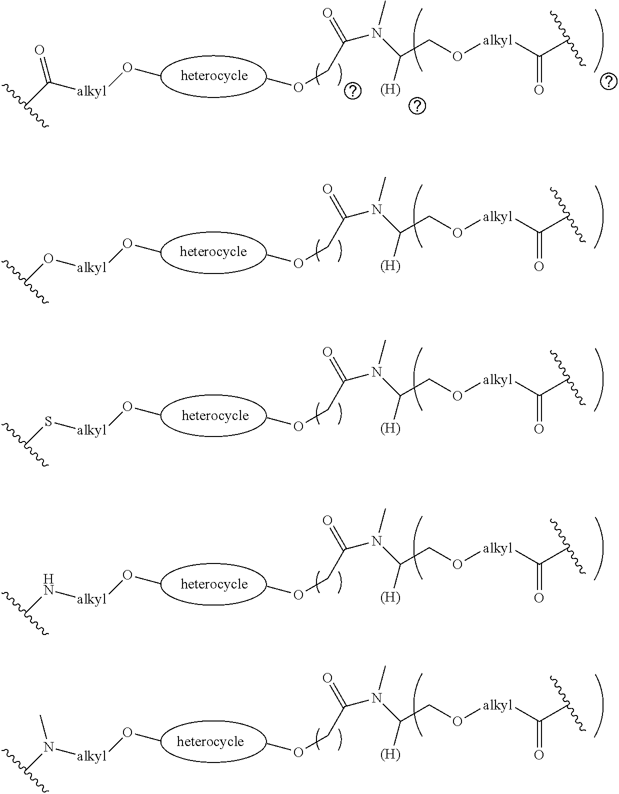

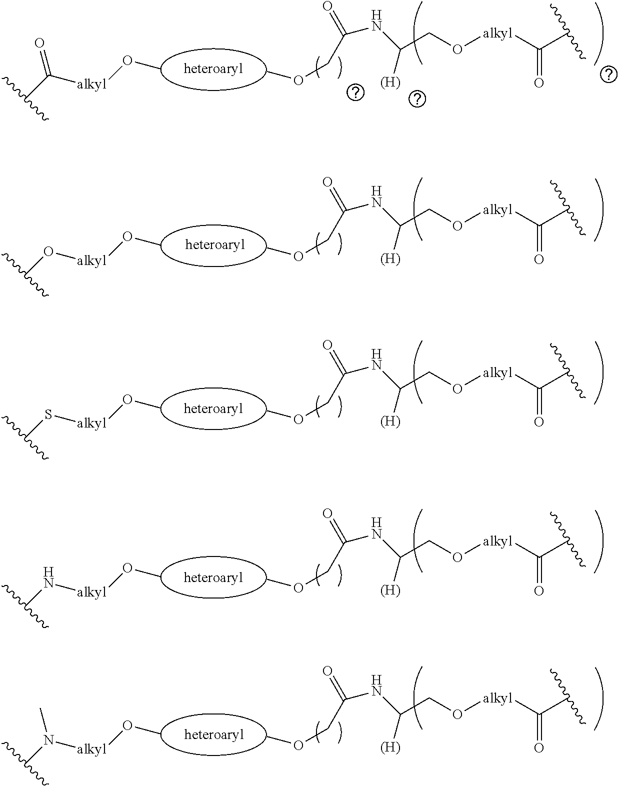

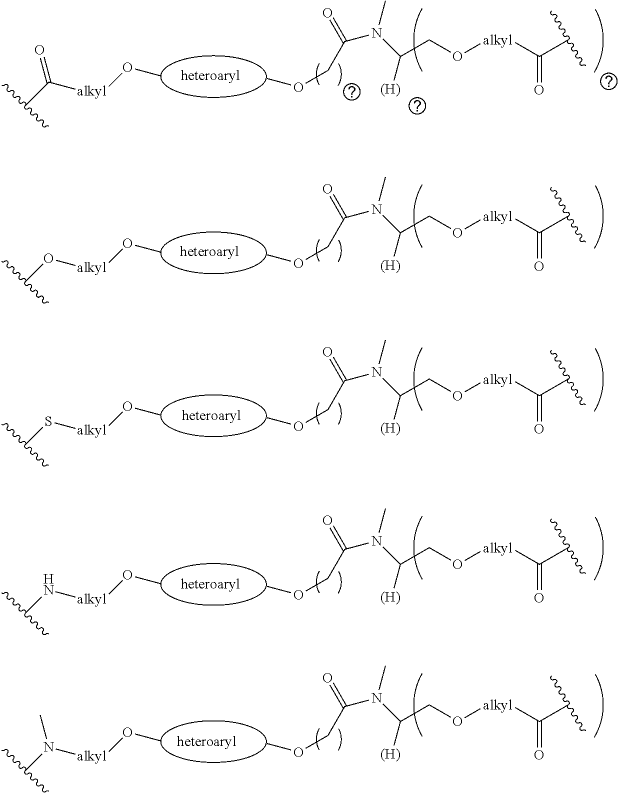

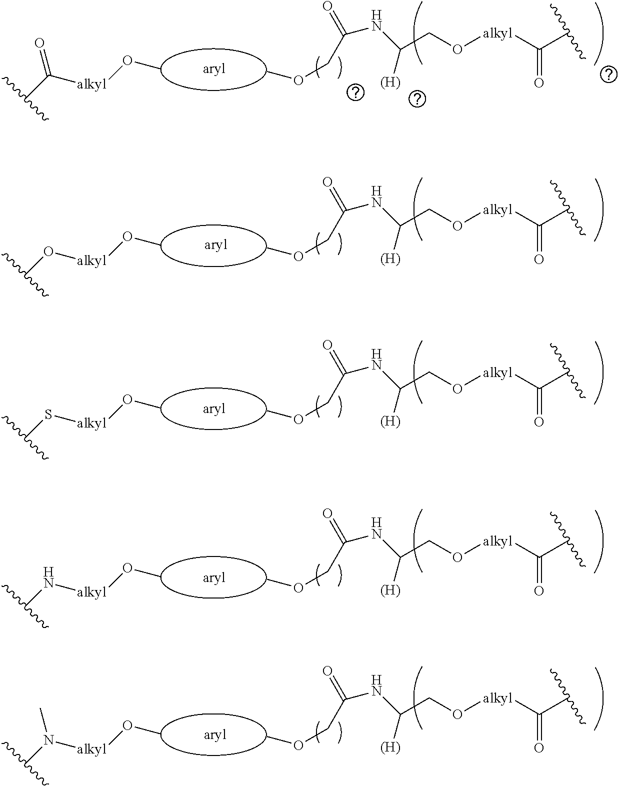

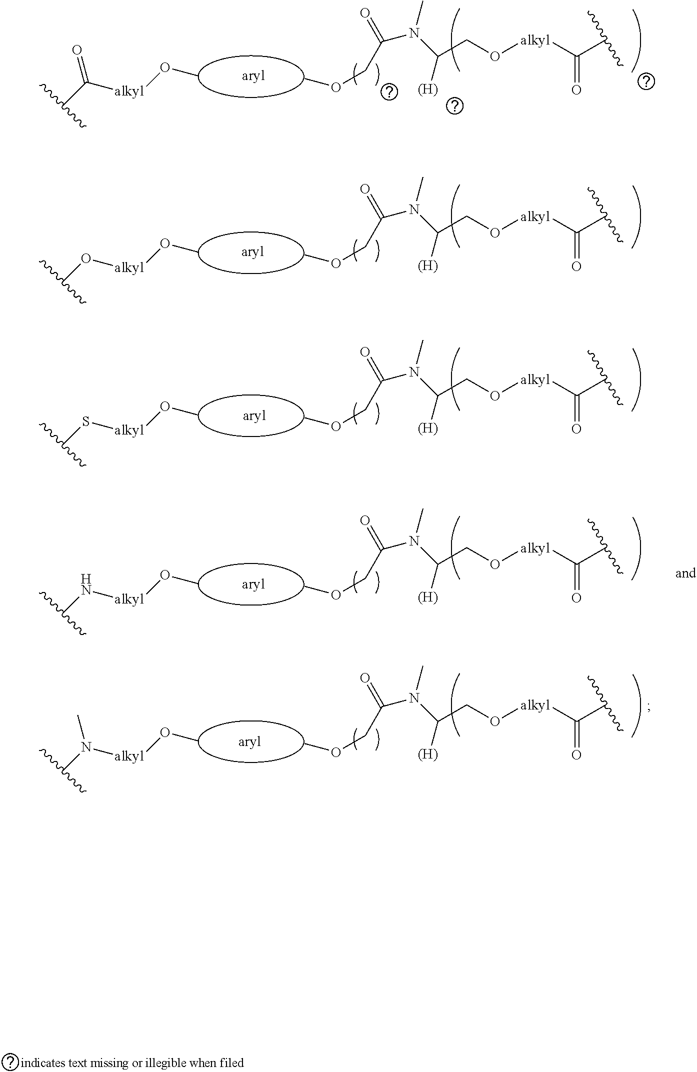

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{B}, Linker^{C}, or Linker^{D} is selected from:

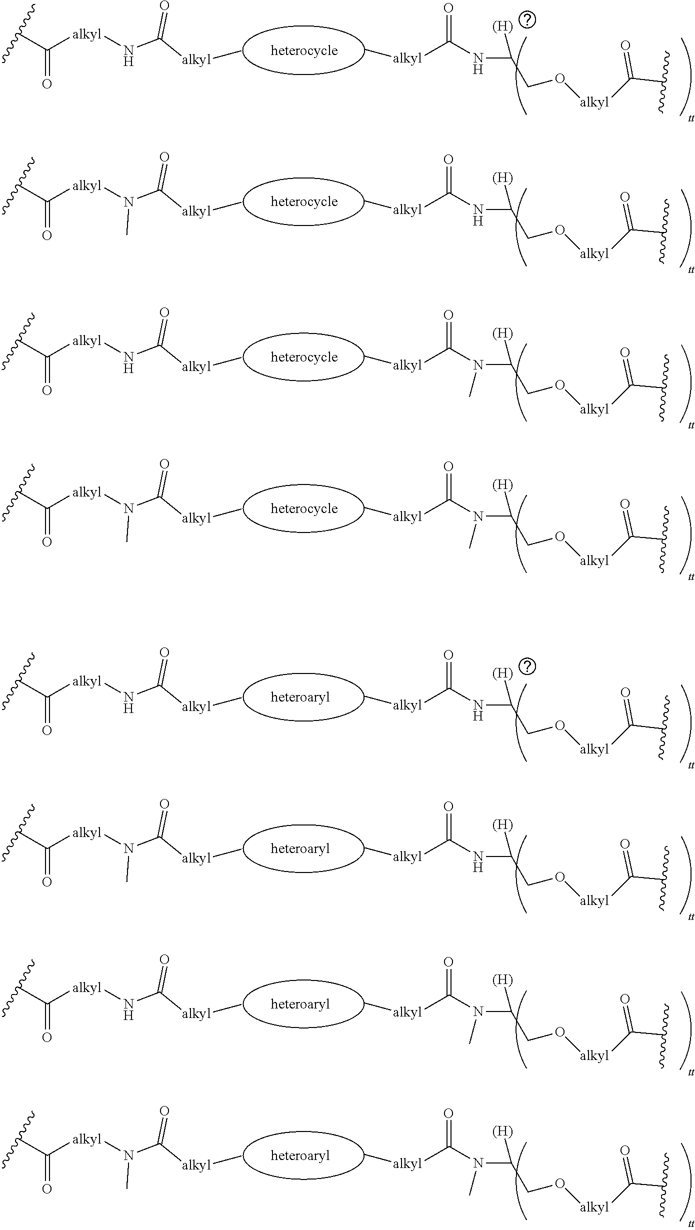

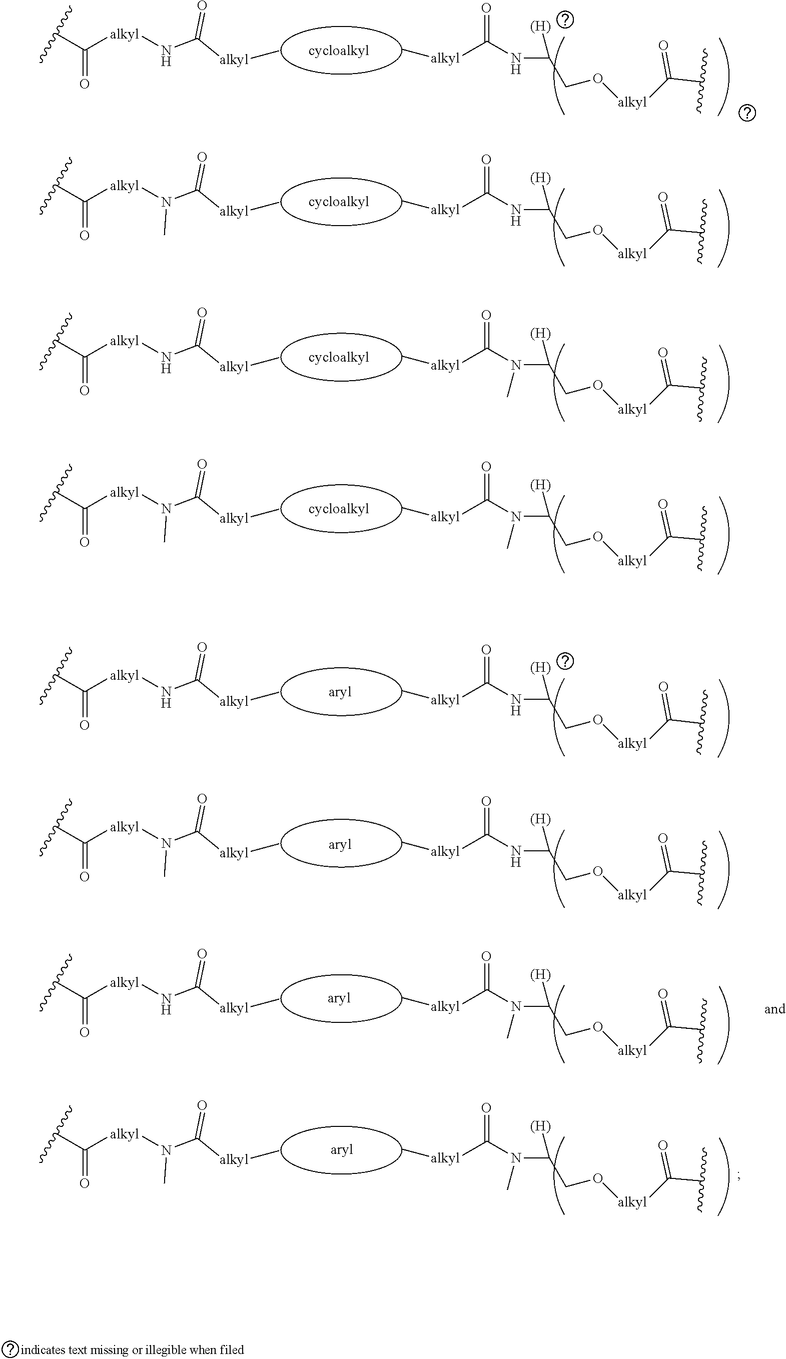

wherein each heteroaryl, heterocycle, cycloalkyl, and aryl can optionally be substituted with 1, 2 3, or 4 of any combination of halogen, alkyl, haloalkyl, and, heteroaryl, heterocycle, or cycloalkyl, as allowed by valence: and tt and ss are as defined herein.

In certain embodiments, in the compound of Formula II, Linker^{B}, Linker^{C}, or Linker^{D} is selected from:

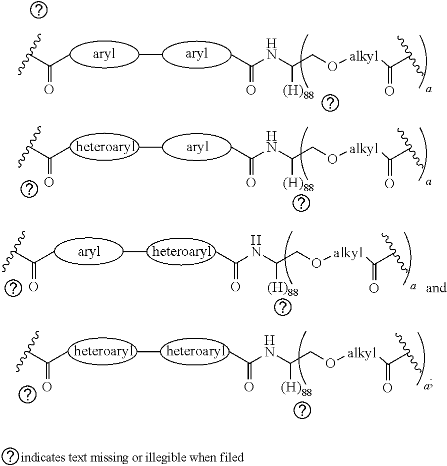

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{A} is selected from:

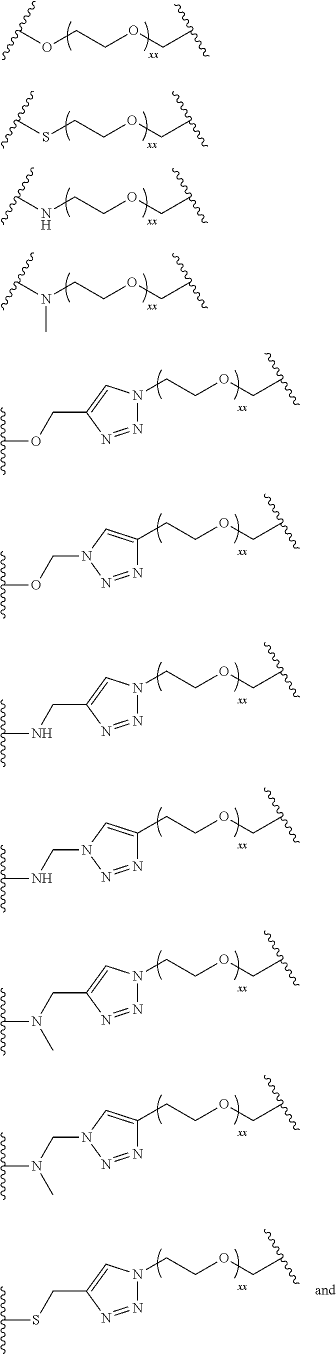

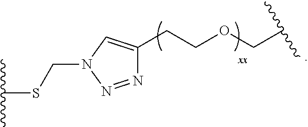

In certain embodiments, in the compound of Formula II, Linker^{A} is selected from:

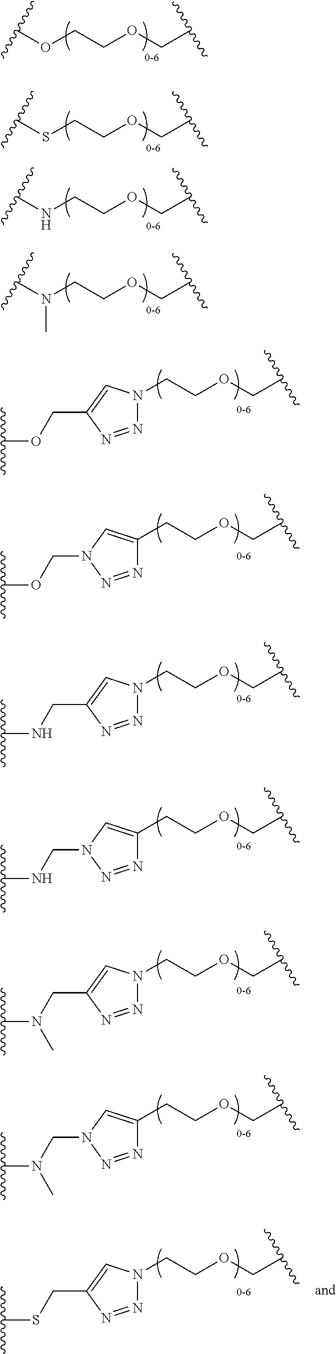

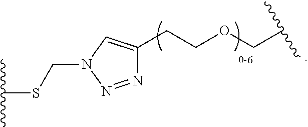

In certain embodiments, in the compound of Formula II, Linker^{A} is selected from:

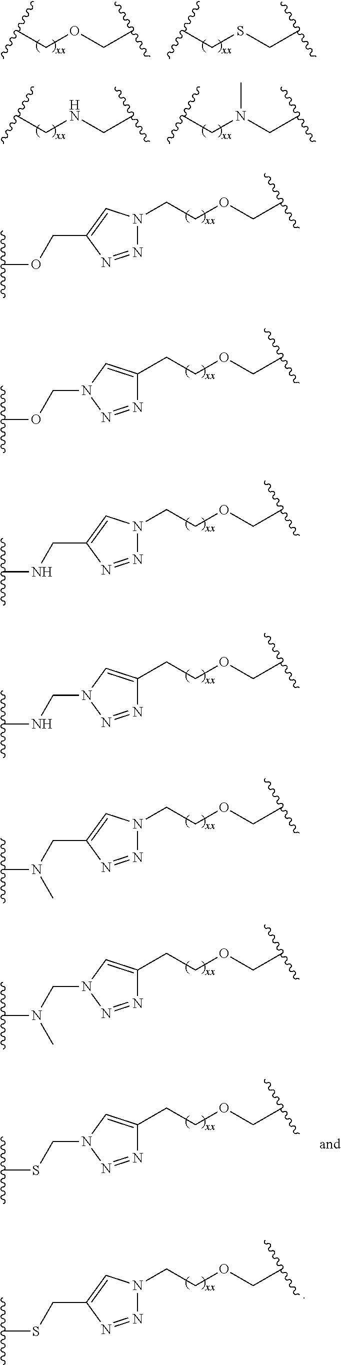

In certain embodiments, in the compound of Formula II, Linker^{A} is selected from:

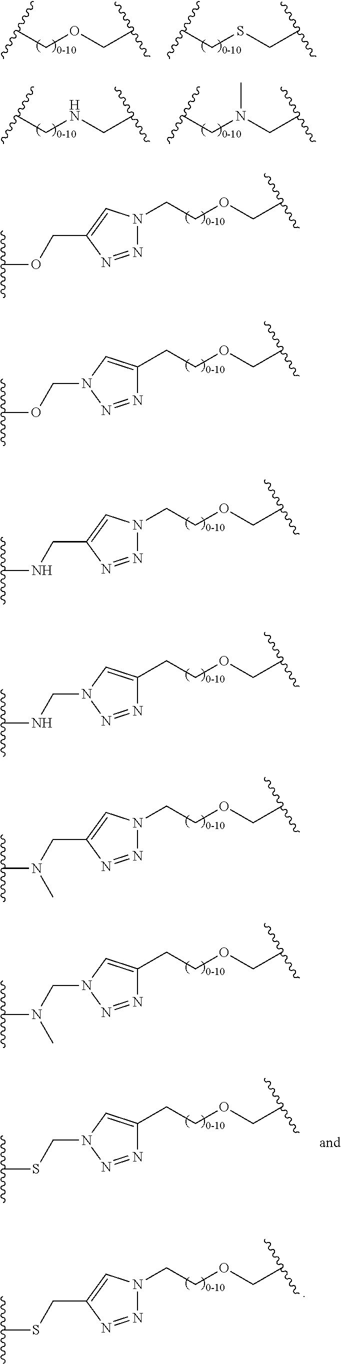

In certain embodiments, in the compound of Formula II, Linker^{B} is selected from:

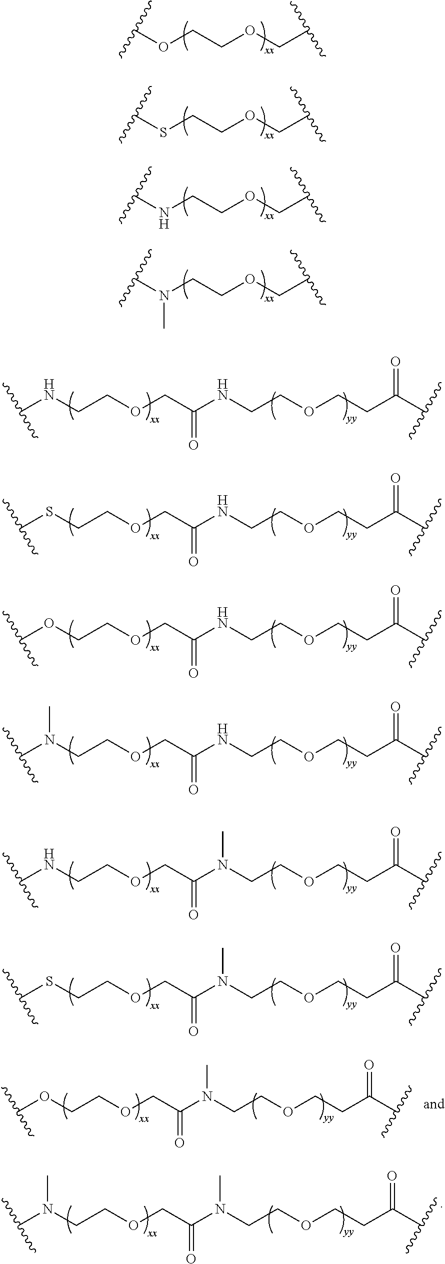

In certain embodiments, in the compound of Formula II, Linker^{B} is selected from:

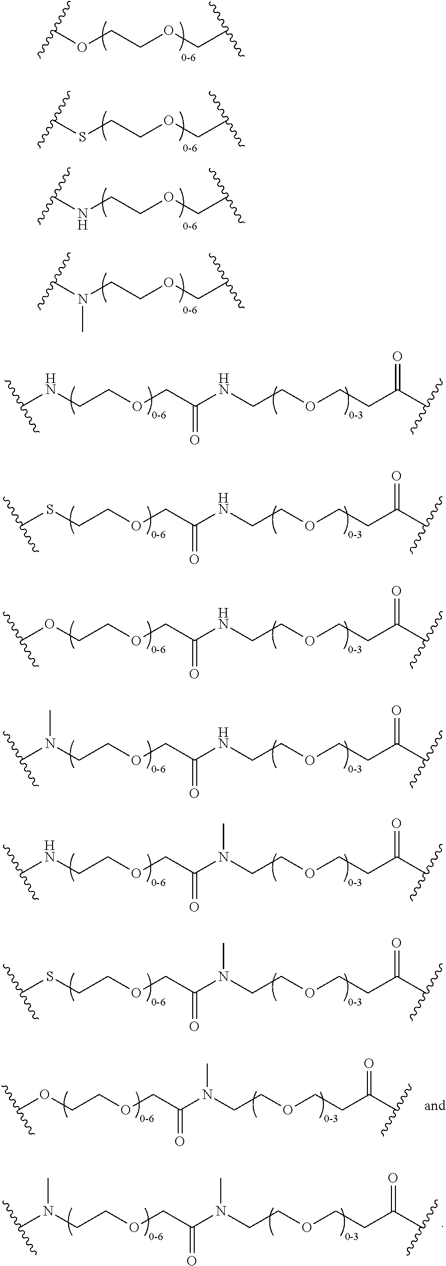

In certain embodiments, in the compound of Formula II, Linker^{B} is selected from:

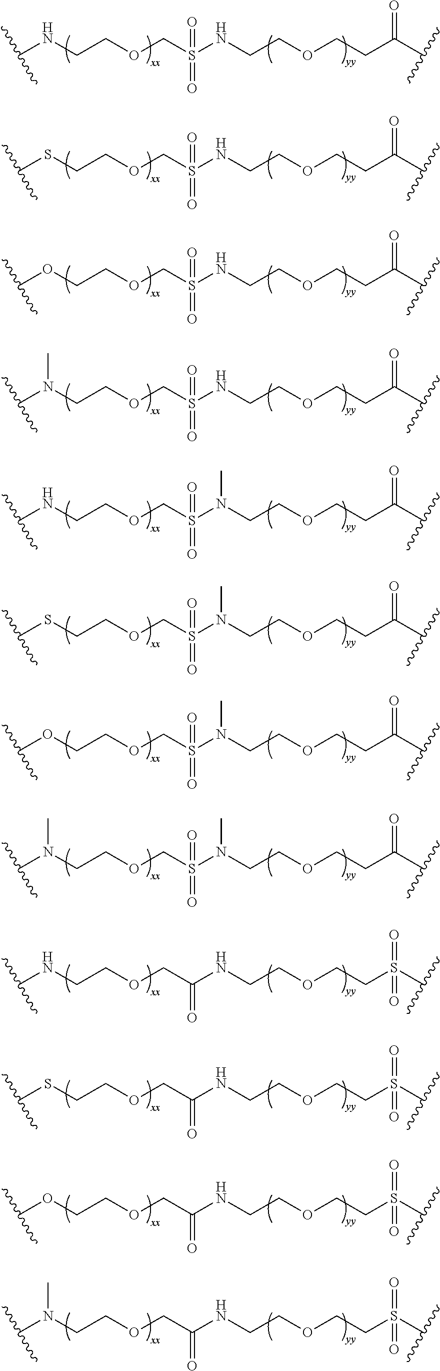

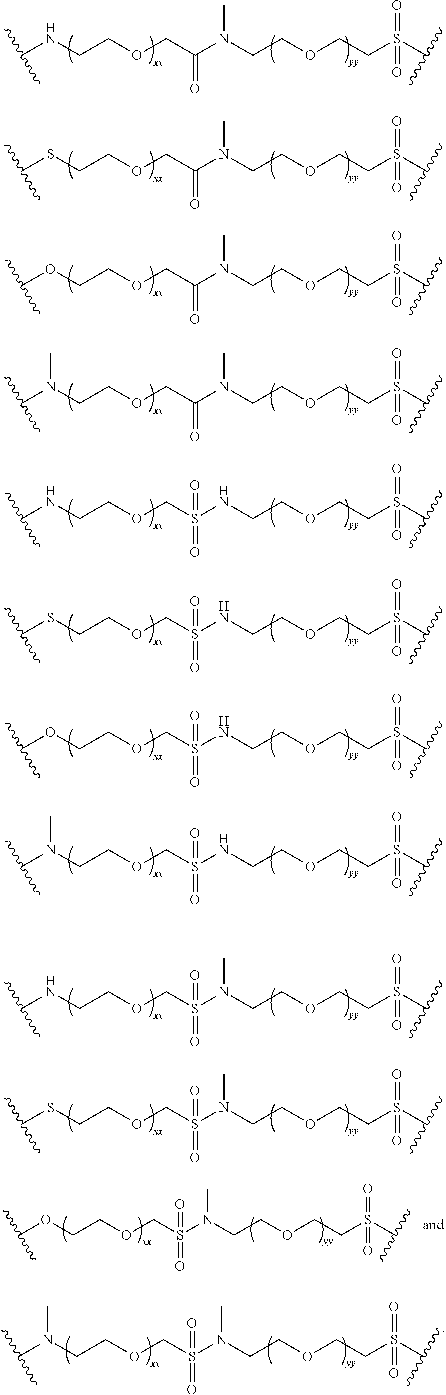

In certain embodiments, in the compound of Formula II, Linker^{B} is selected from:

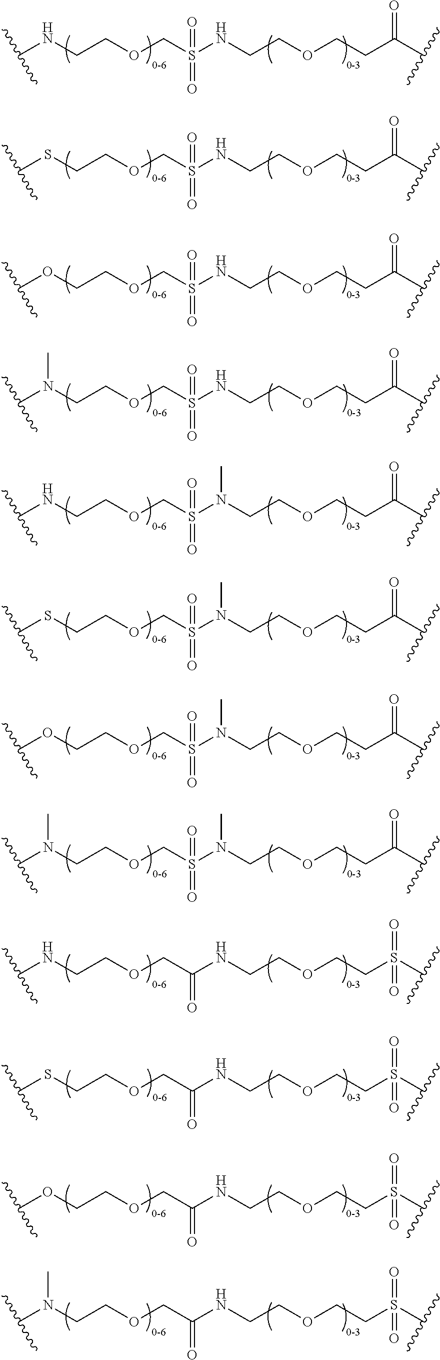

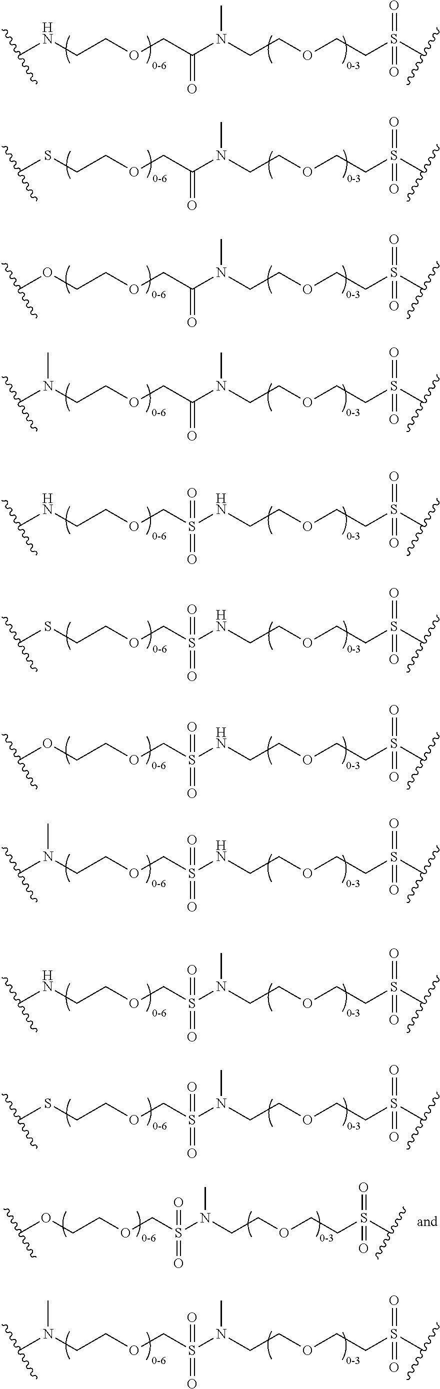

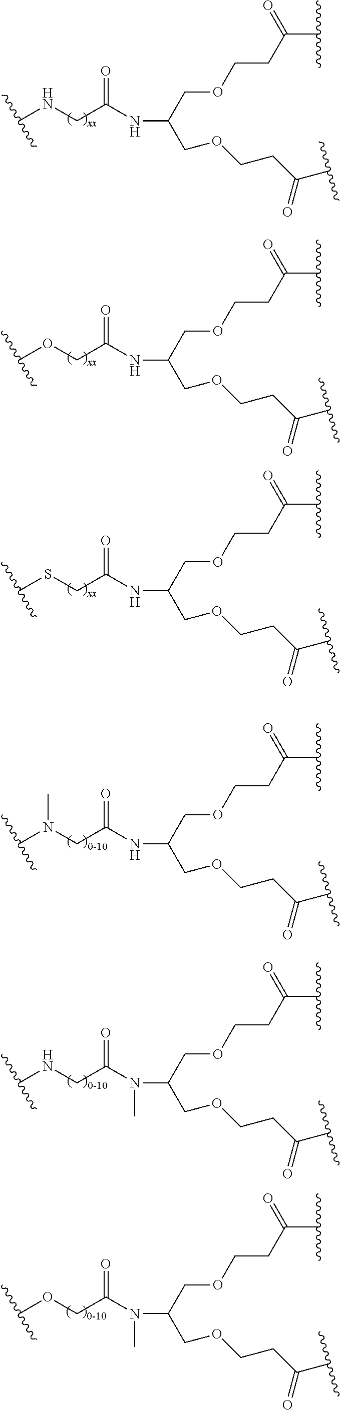

In certain embodiments, in the compound of Formula II, Linker^{C} is selected from:

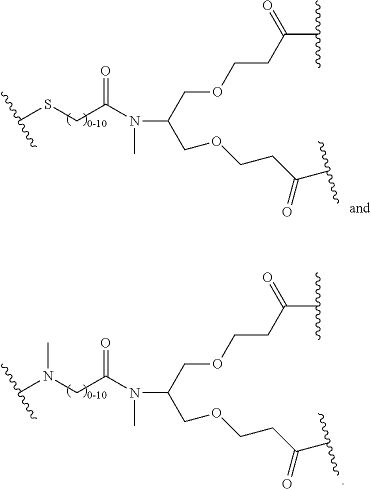

In certain embodiments, in the compound of Formula II, Linker^{C} is selected from:

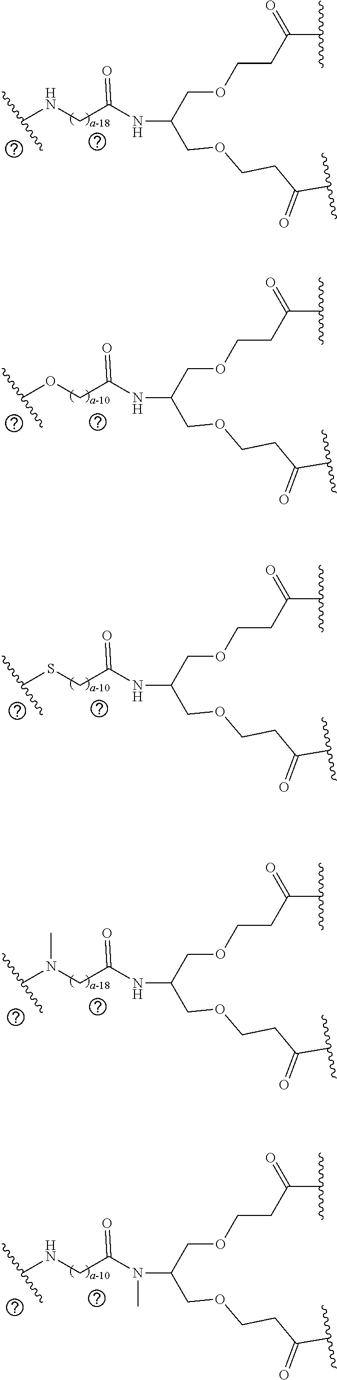

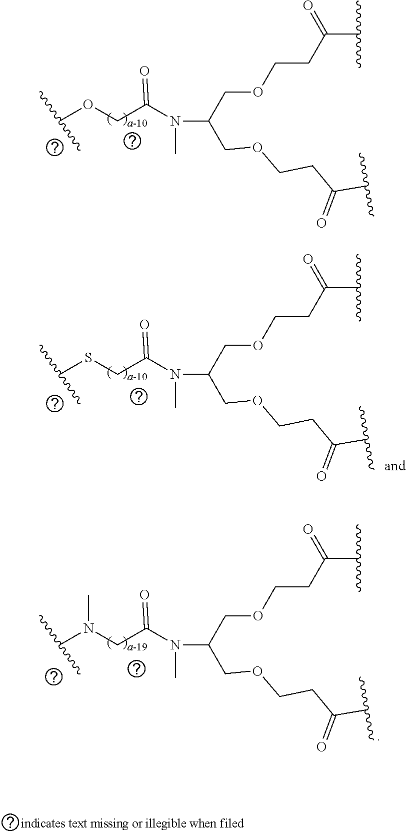

In certain embodiments, in the compound of Formula II, Linker^{C} is selected from:

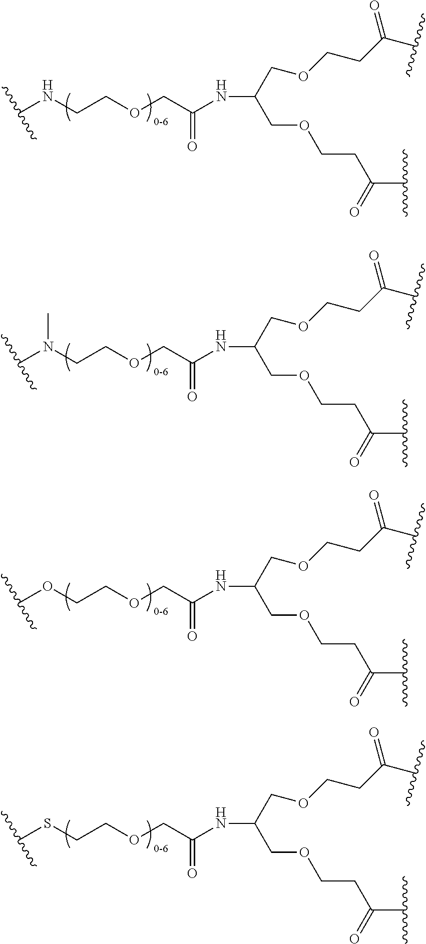

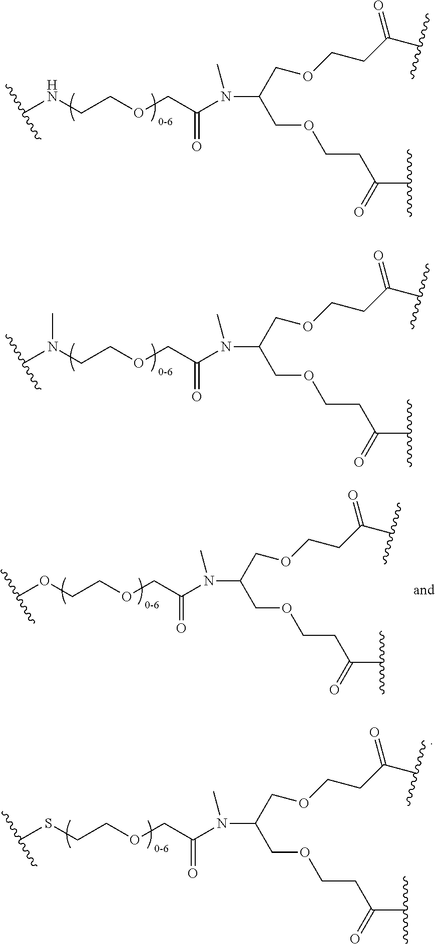

In certain embodiments, in the compound of Formula II, Linker^{C} is selected from:

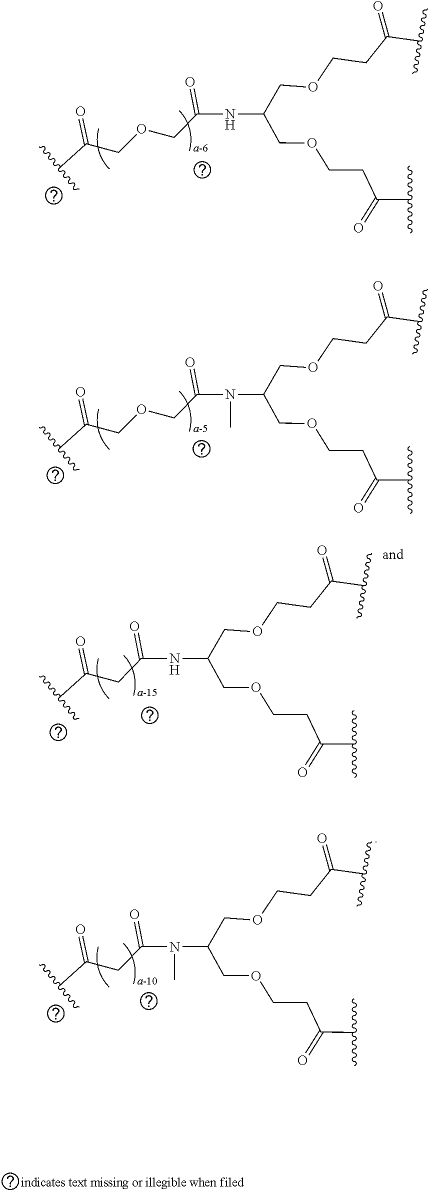

In certain embodiments, in the compound of Formula II, Linker^{C} is selected from:

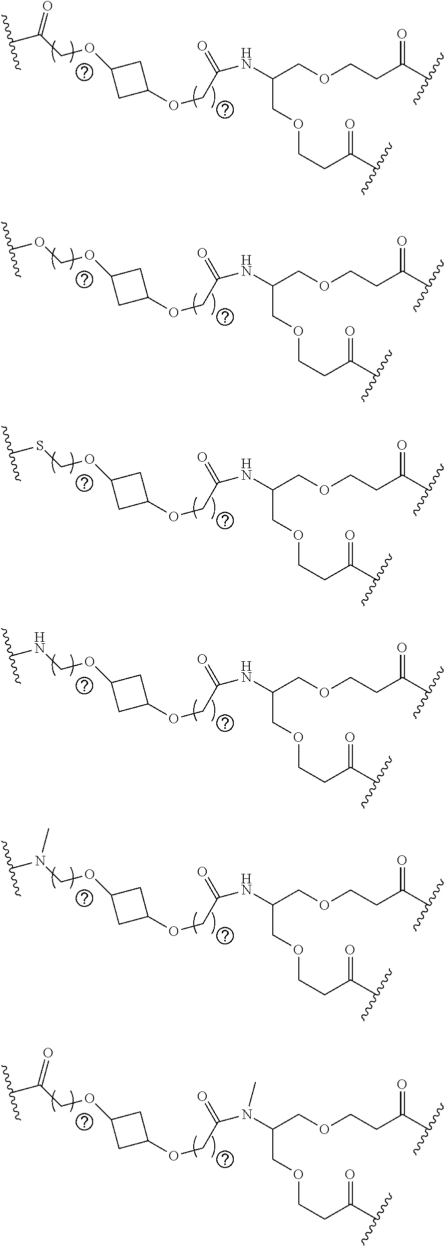

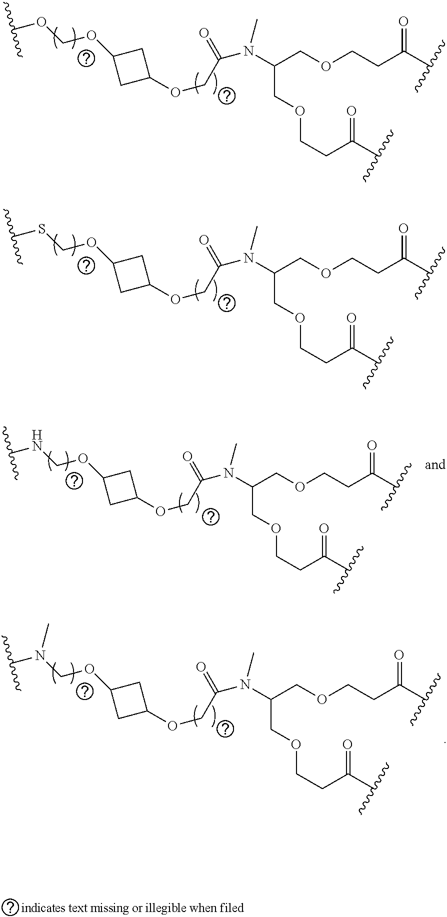

In certain embodiments, in the compound of Formula II, Linker^{C} is selected from:

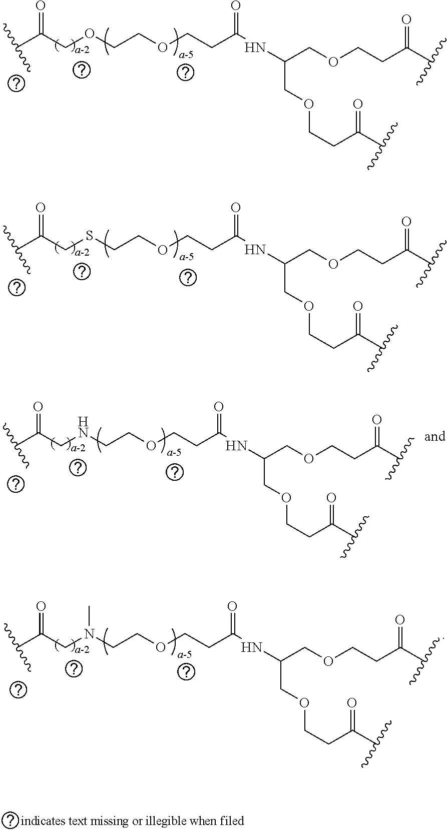

In certain embodiments, in the compound of Formula II, Linker^{C} is selected from:

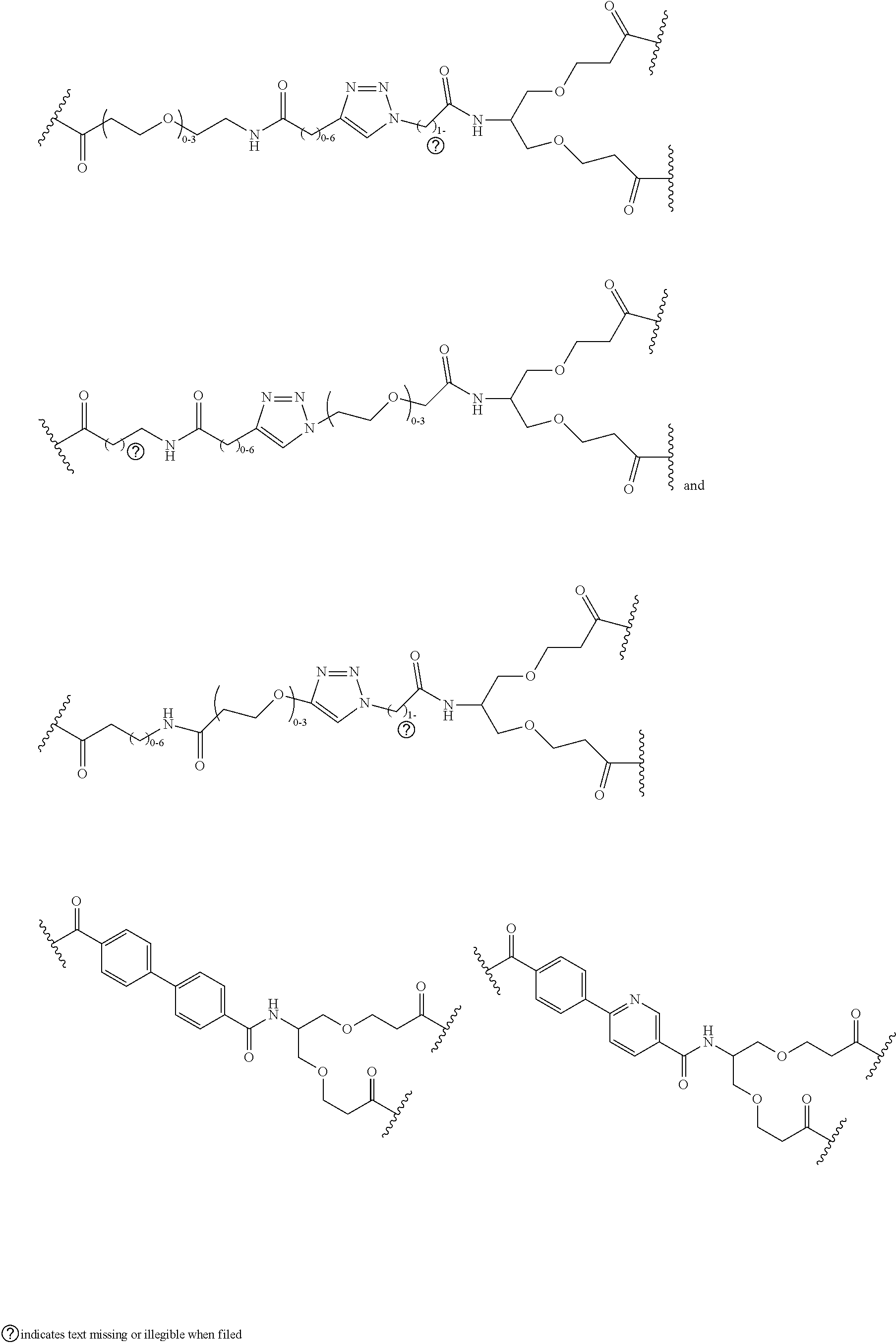

In certain embodiments, in the compound of Formula II, Linker^{C} is selected from:

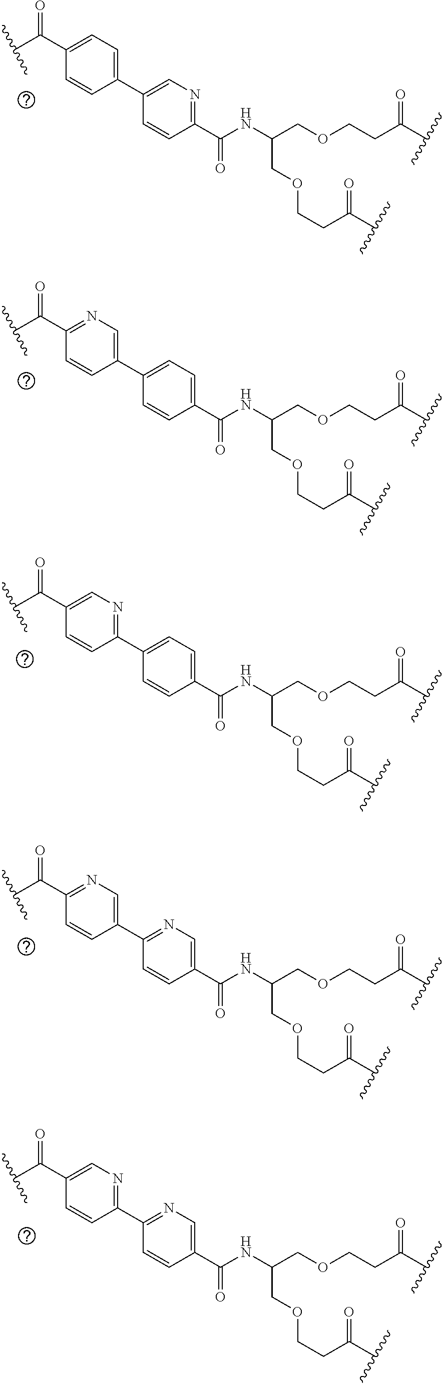 [SMILES: *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1 ; *C(=O)CCOCC(COCCC(*)=O)NC(=O)c1ccc(-c2ccc(C(*)=O)cn2)cc1 ; *C(=O)CCOCC(COCCC(*)=O)NC(=O)c1ccc(-c2ccc(C(*)=O)nc2)nc1 ; *C(=O)CCOCC(COCCC(*)=O)NC(=O)c1ccc(-c2ccc(C(*)=O)cn2)nc1]

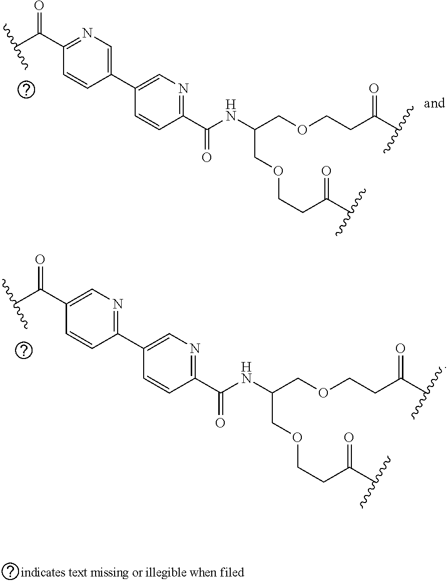 [SMILES: *C(=O)CCOCC(COCCC(*)=O)NC(=O)c1ccc(-c2ccc(C(*)=O)nc2)cn1 ; *C(=O)CCOCC(COCCC(*)=O)NC(=O)c1ccc(-c2ccc(C(*)=O)cn2)cn1]

In certain embodiments, in the compound of Formula II, Linker^{D} is selected from:

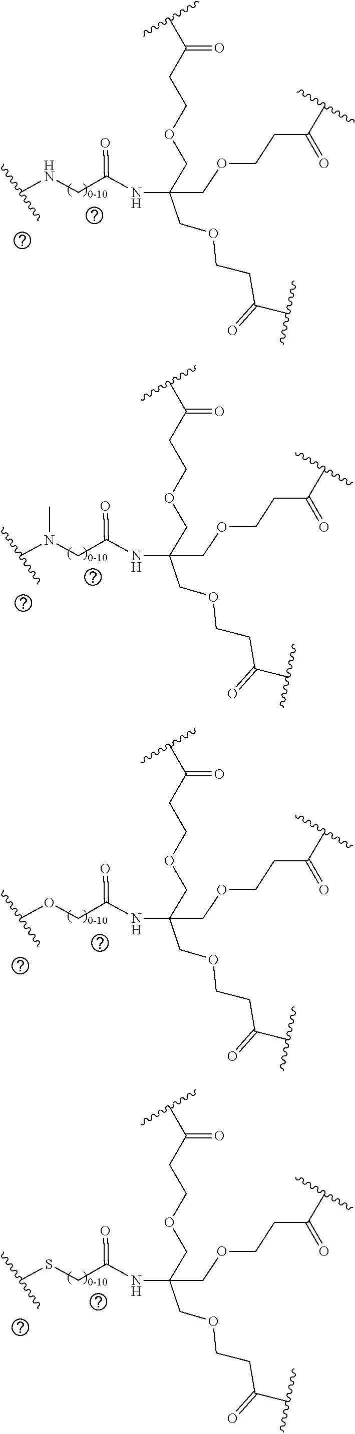

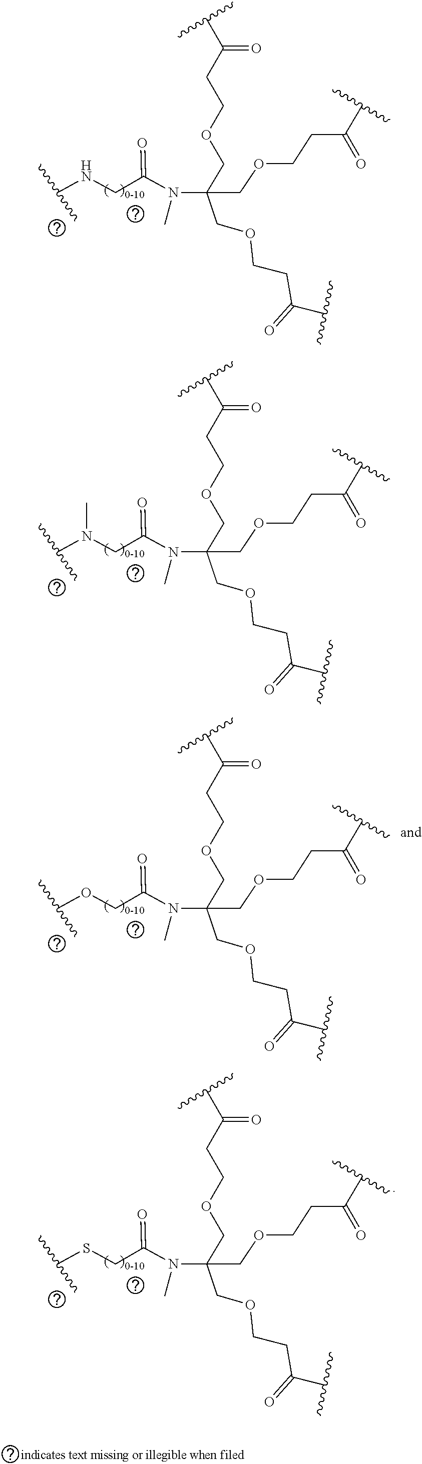

In certain embodiments, in the compound of Formula II, Linker^{D} is selected from:

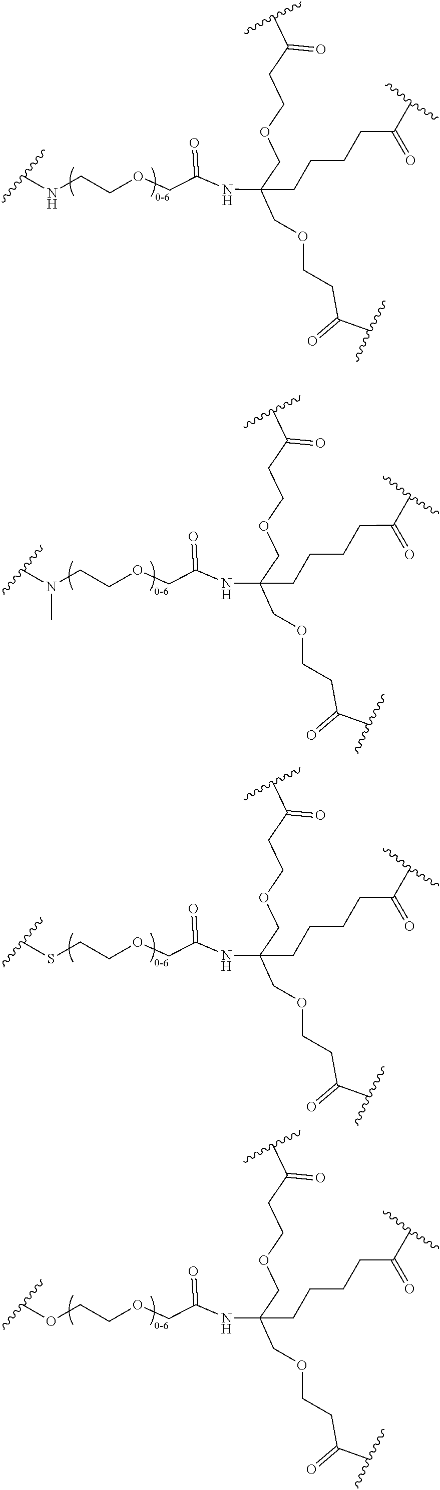

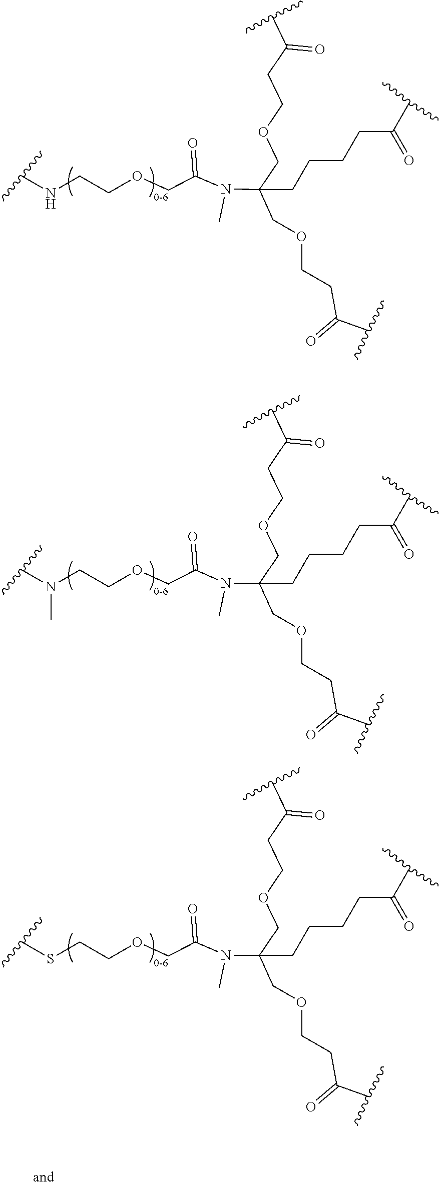

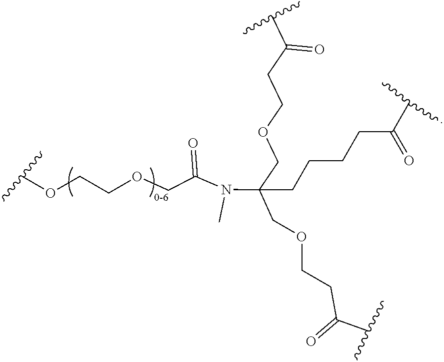

In certain embodiments, in the compound of Formula II, LinkerD is selected from:

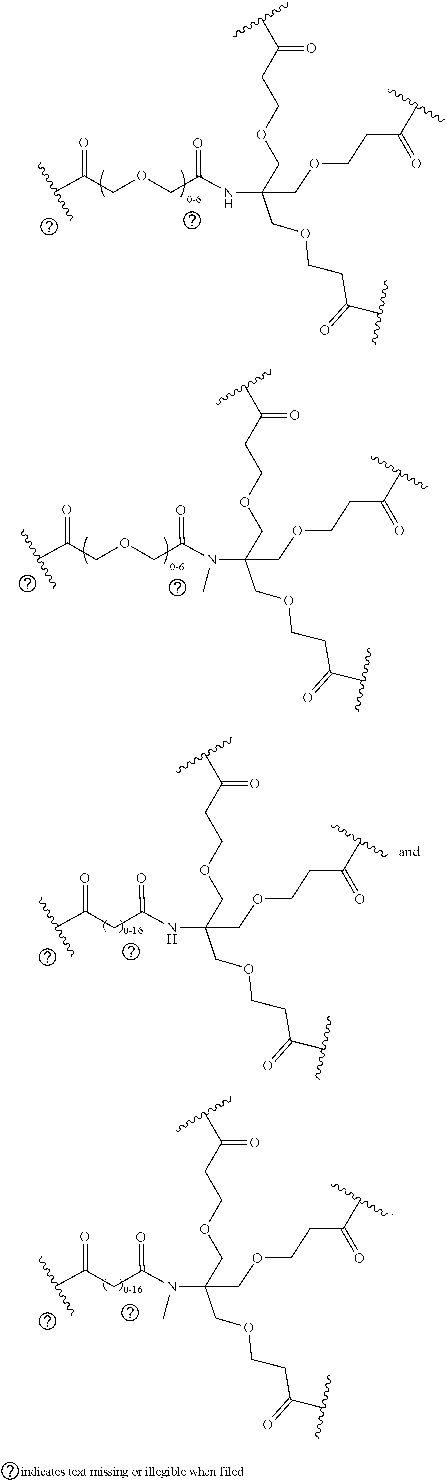

In certain embodiments, in the compound of Formula II, Linker^{D} is selected from:

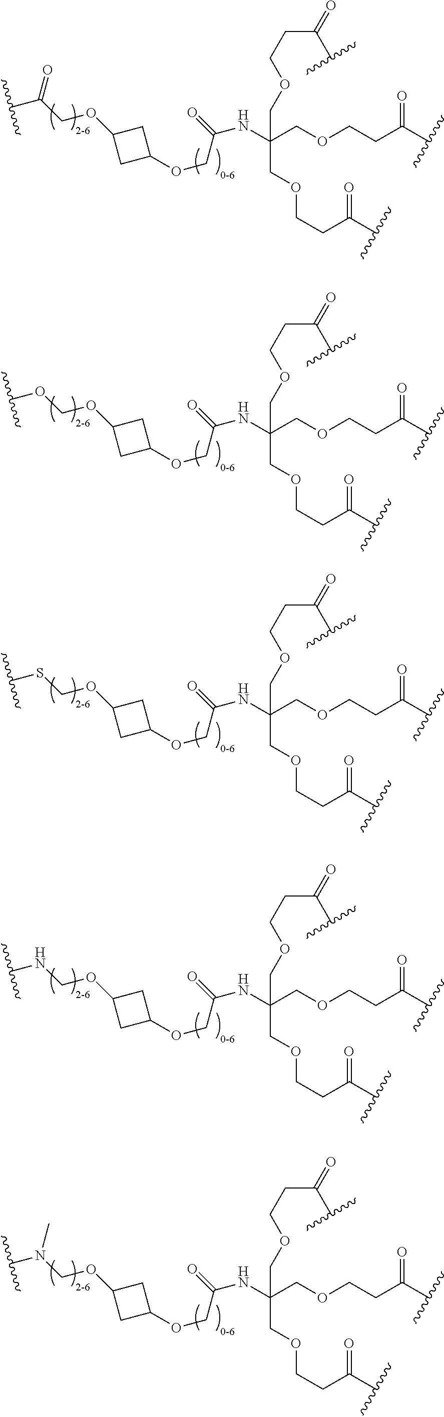

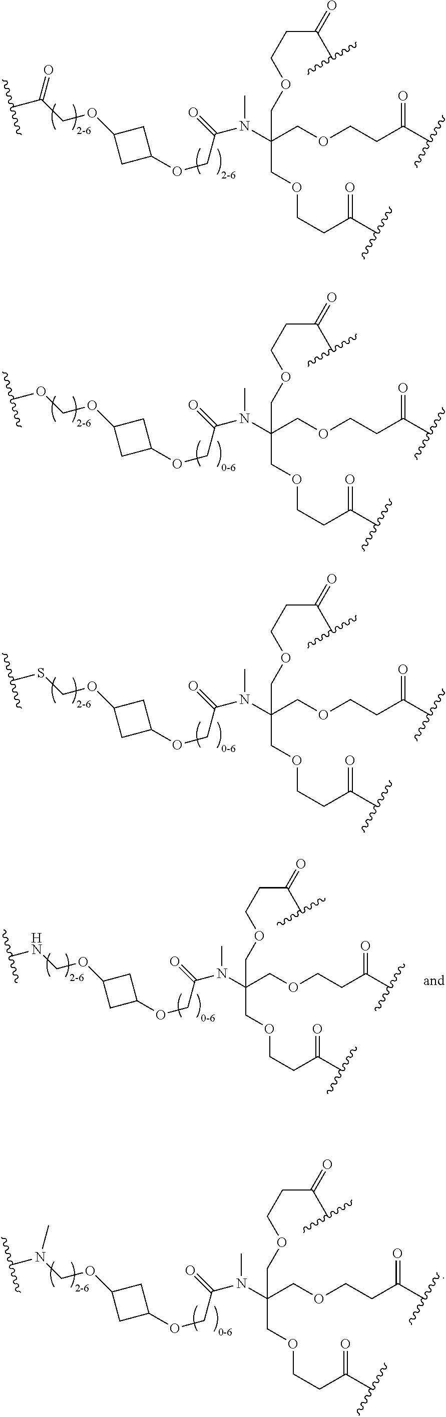

In certain embodiments, in the compound of Formula II, Linker^{D} is selected from:

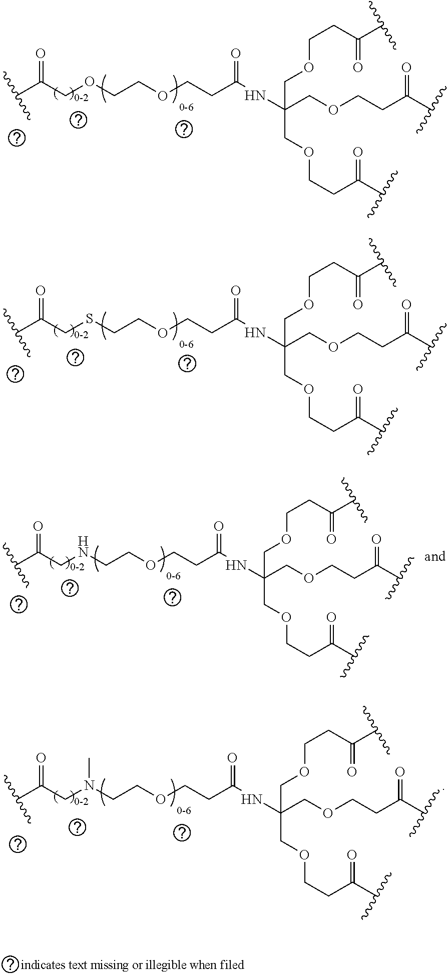

In certain embodiments, in the compound of Formula II, Linker^{D} is selected from:

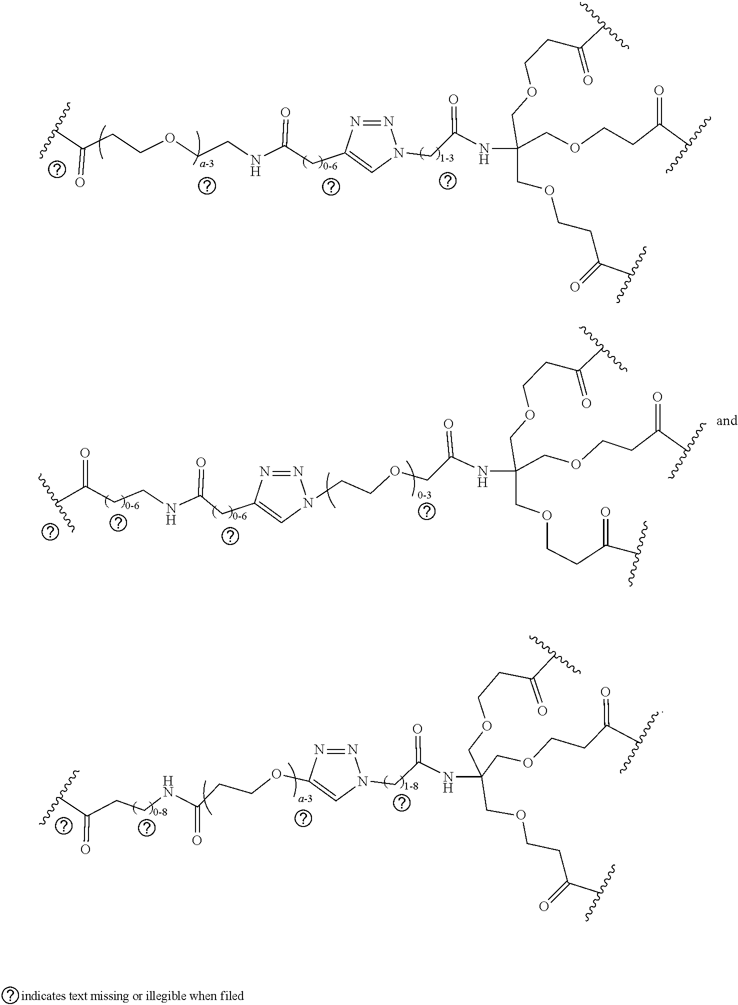

In certain embodiments, in the compound of Formula II, Linker^{D} is selected from:

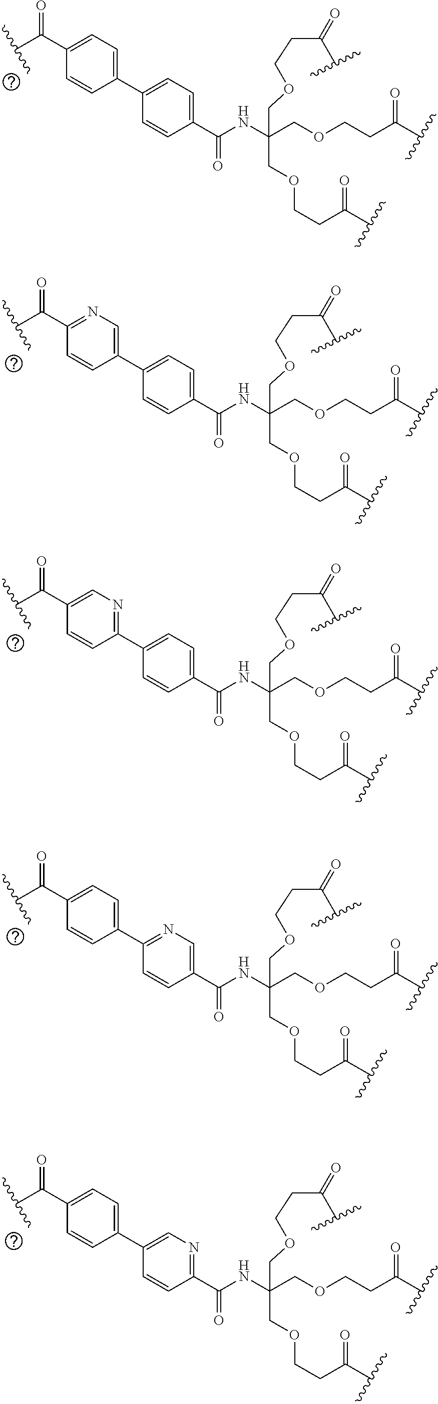 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1 ; *C(=O)CCOCC(COCCC(*)=O)(COCCC(*)=O)NC(=O)c1ccc(-c2ccc(C(*)=O)cc2)cn1]

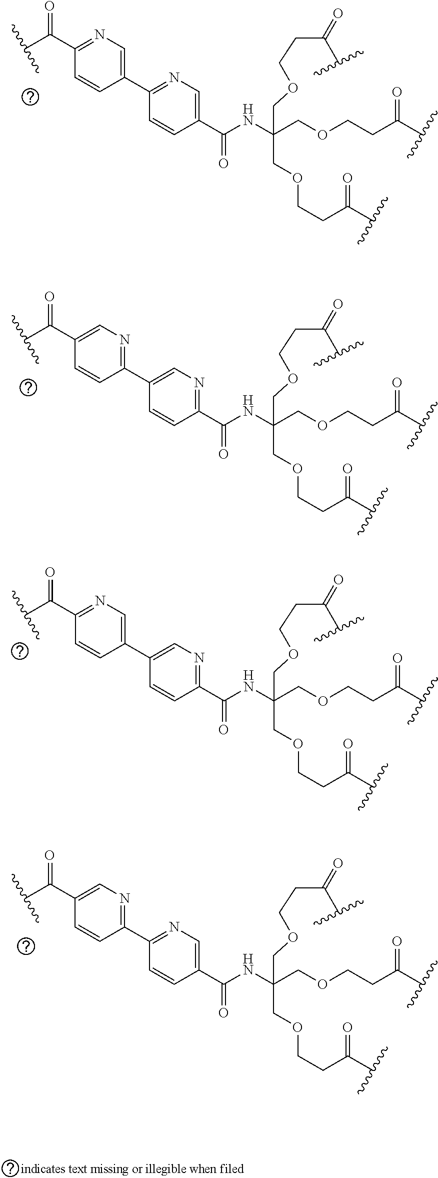 [SMILES: *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1 ; *C(=O)CCOCC(COCCC(*)=O)(COCCC(*)=O)NC(=O)c1ccc(-c2ccc(C(*)=O)cn2)nc1]

In certain embodiments, in the compound of Formula II, the Linker^{A} is selected from

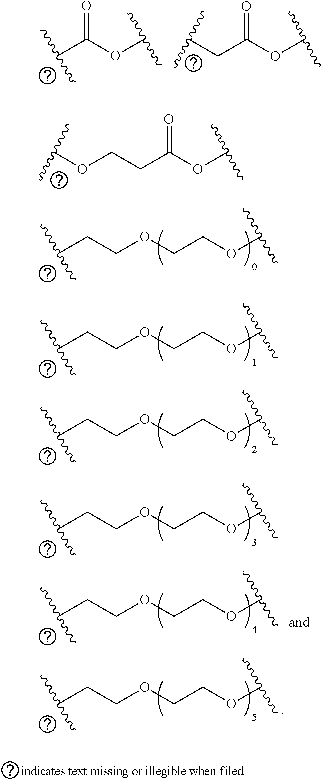

In certain embodiments, in the compound of Formula II, the Linker^{A} is selected from

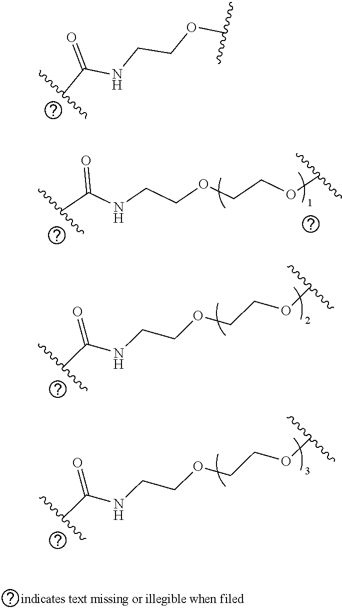

In certain embodiments, in the compound of Formula II, the Linker^{A} is selected from

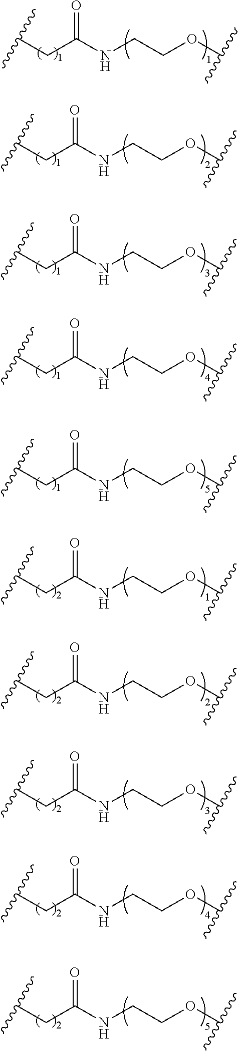

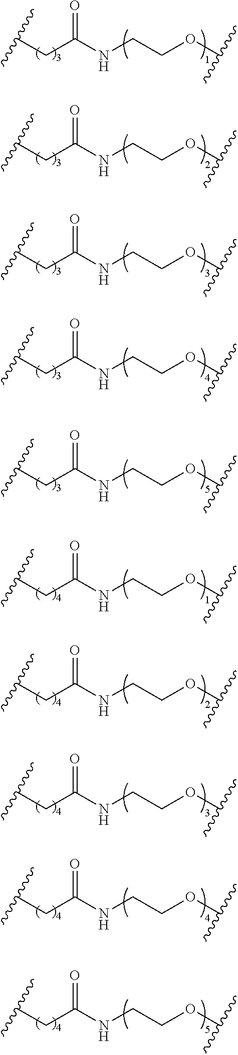

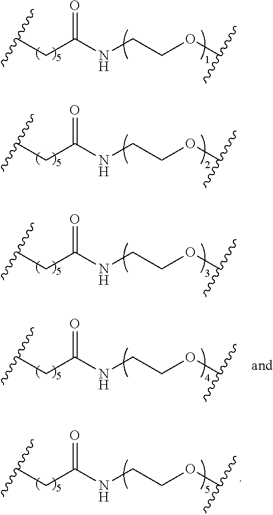

In certain embodiments, the Linker^{A} is selected from

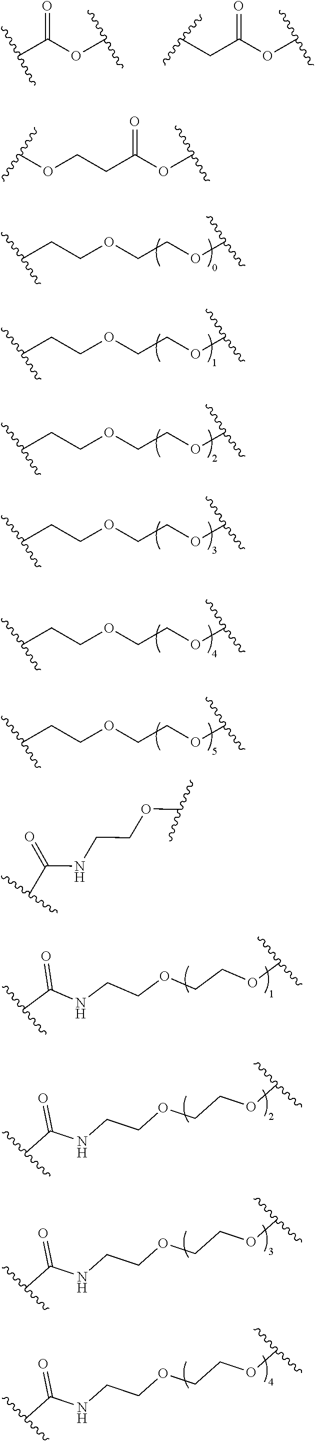

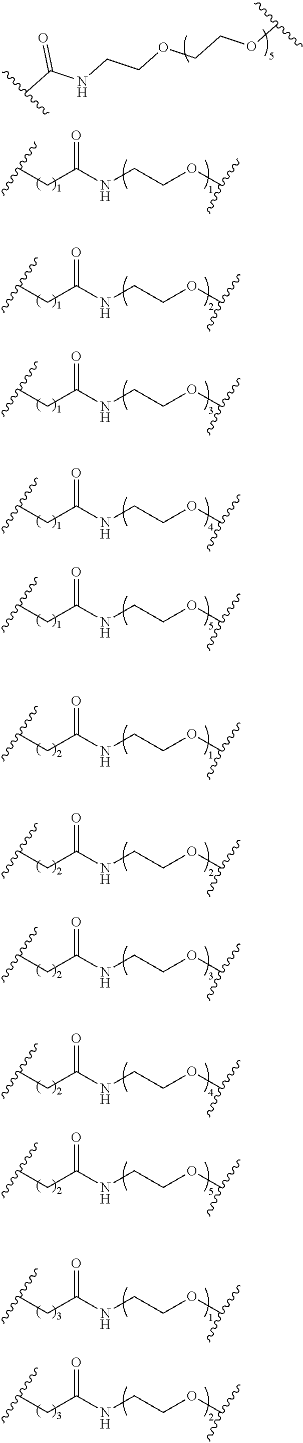

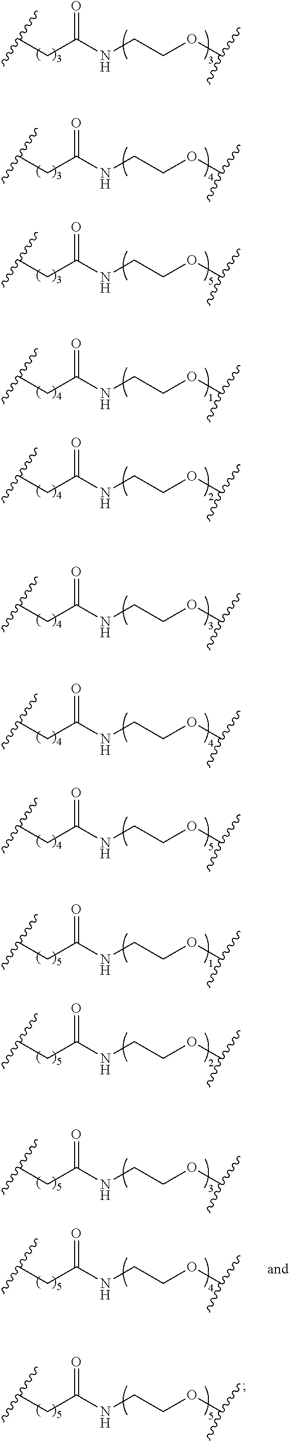

wherein each is optionally substituted with 1, 2, 3, or 4 substituents substituent selected from R²¹.

In certain embodiments, in the compound of Formula II, Linker^{A} is selected from:

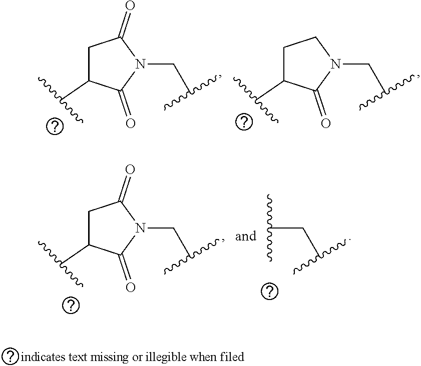

In certain embodiments, in the compound of Formula II, the Linker^{A} is selected from

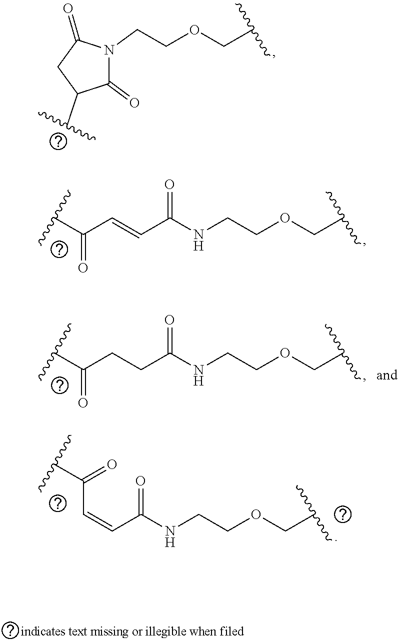 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments, in the compound of Formula II, the Linker^{A} is selected from

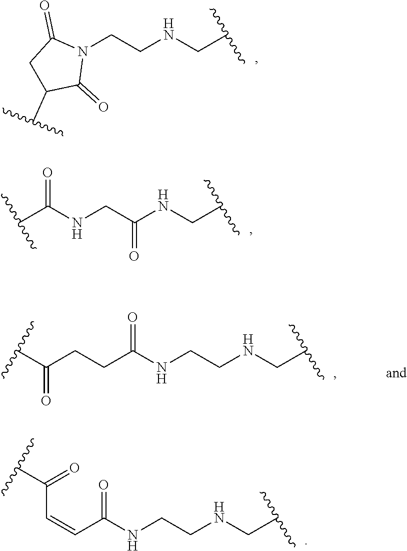 [SMILES: *CNCCN1C(=O)CC(*)C1=O ; *CNC(=O)CNC(*)=O ; *CNCCNC(=O)CCC(*)=O ; *CNCCNC(=O)/C=C\C(*)=O]

In certain embodiments, in the compound of Formula II, the Linker^{A} is selected from

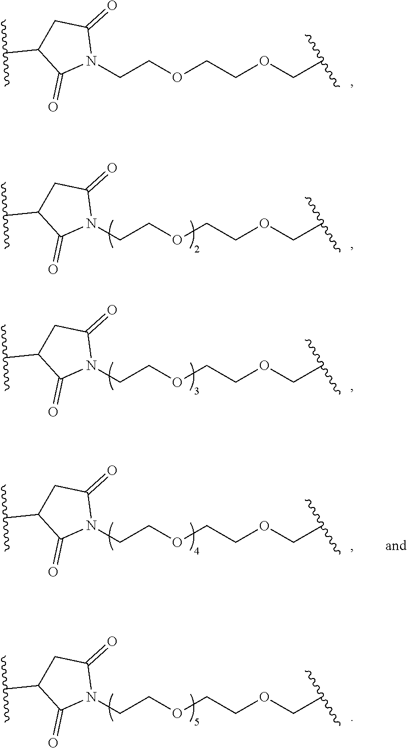

In certain embodiments, in the compound of Formula II, the Linker^{A} is selected from

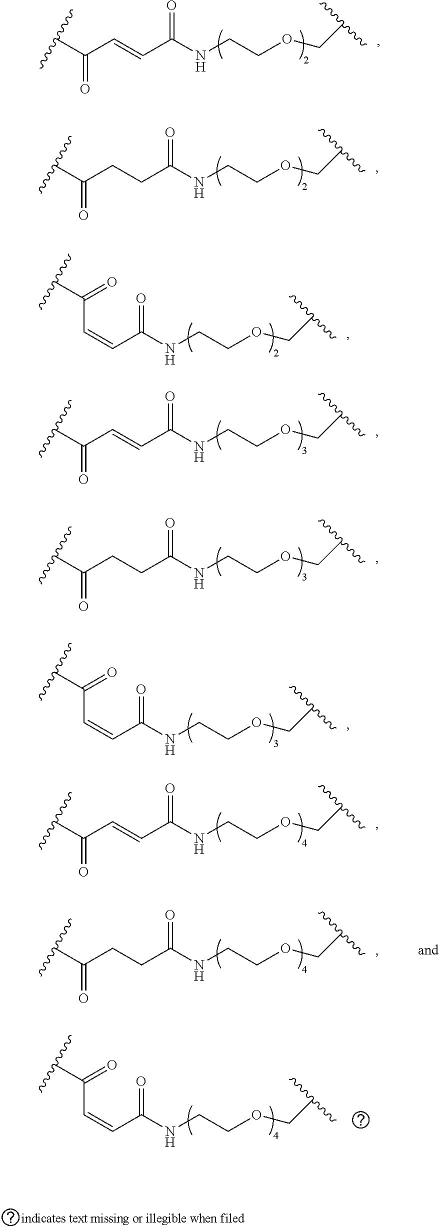

In certain embodiments, in the compound of Formula II, the Linker^{A} is selected from

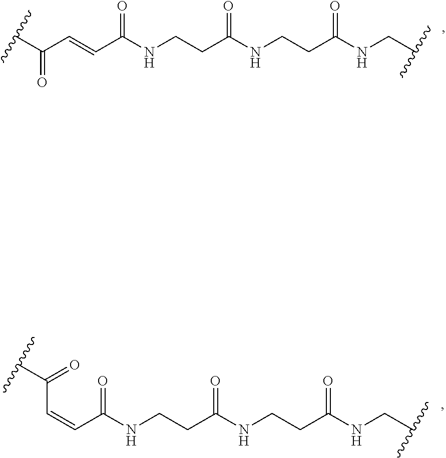 [SMILES: *CNC(=O)CCNC(=O)CCNC(=O)/C=C/C(*)=O ; *CNC(=O)CCNC(=O)CCNC(=O)/C=C\C(*)=O]

In certain embodiments, in the compound of Formula II, the Linker^{A} is selected from

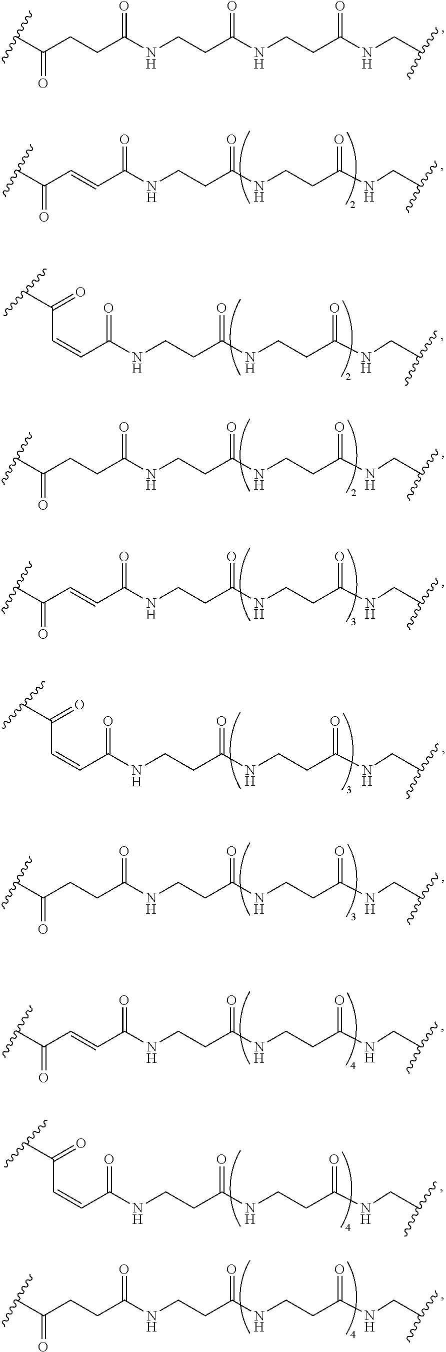

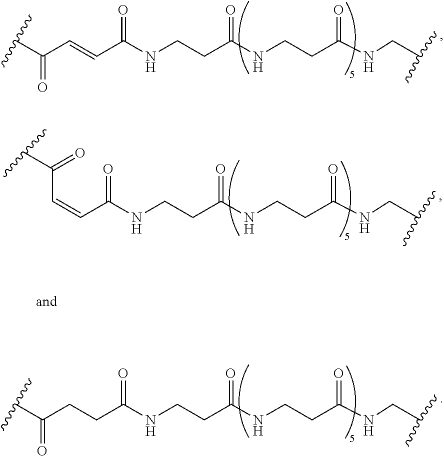

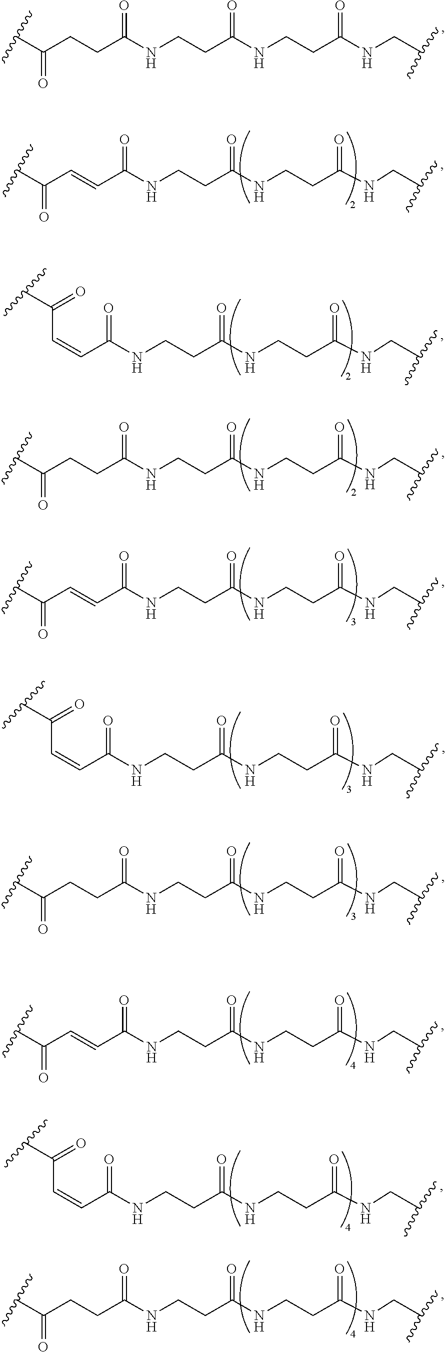

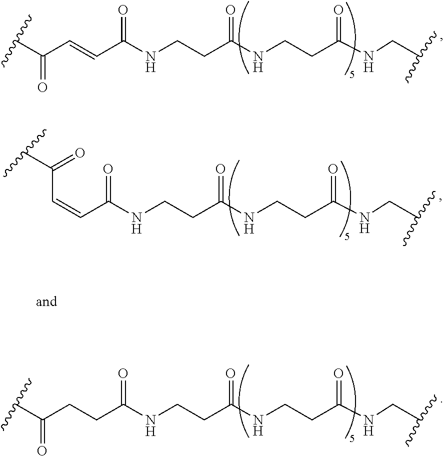

In certain embodiments, in the compound of Formula II, the Linker^{A} is selected from

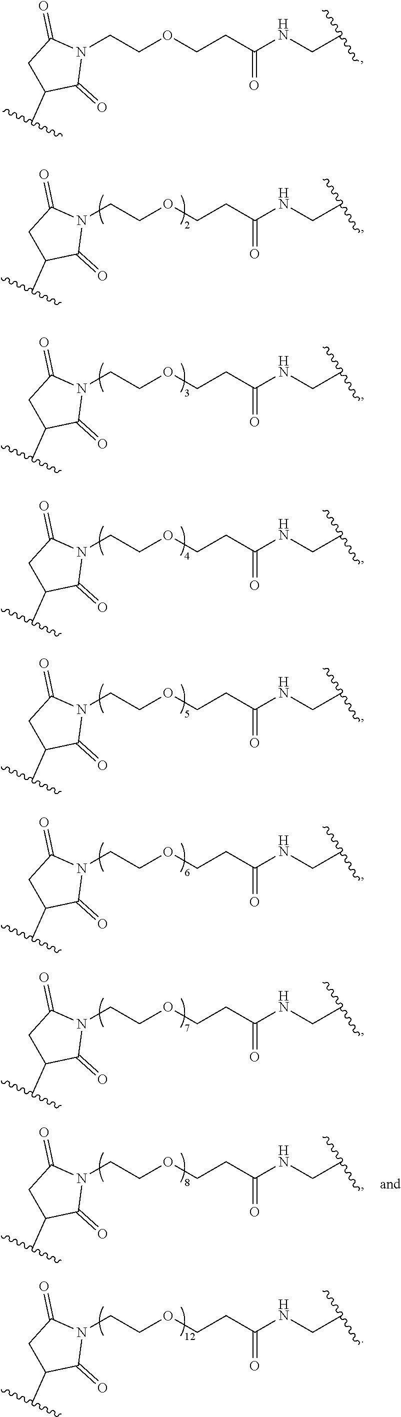

In certain embodiments, in the compound of Formula II, the Linker^{A} is selected from

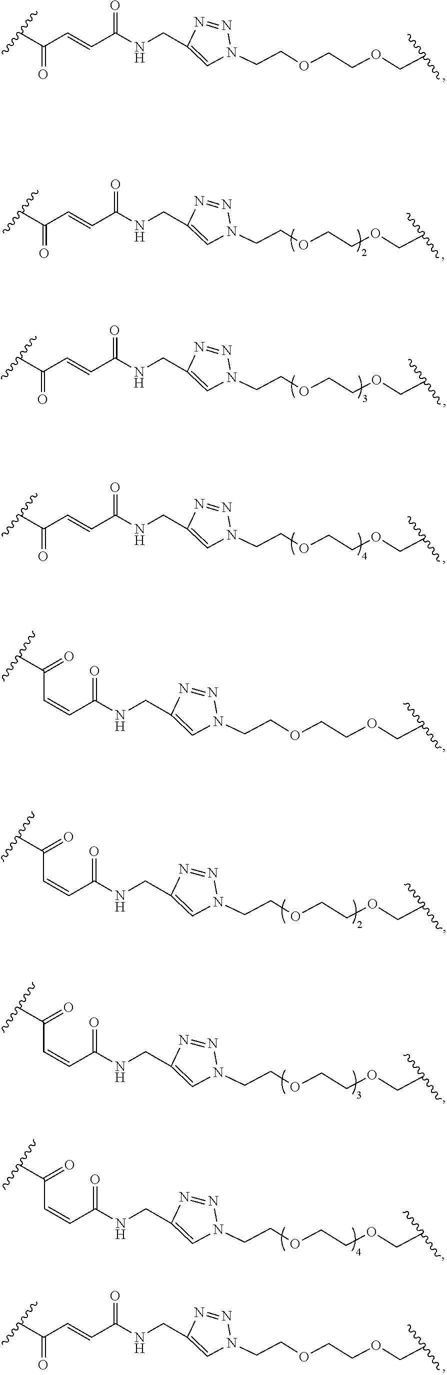

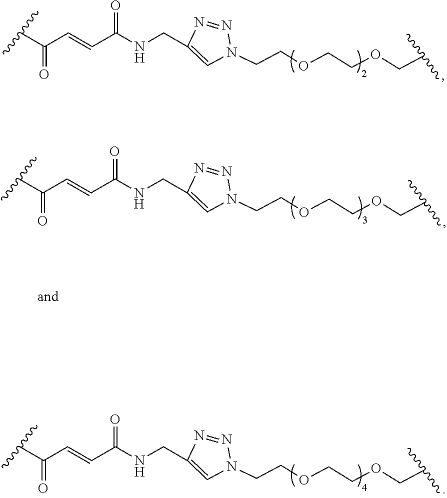

In certain embodiments, in the compound of Formula II, the Linker^{A} is selected from

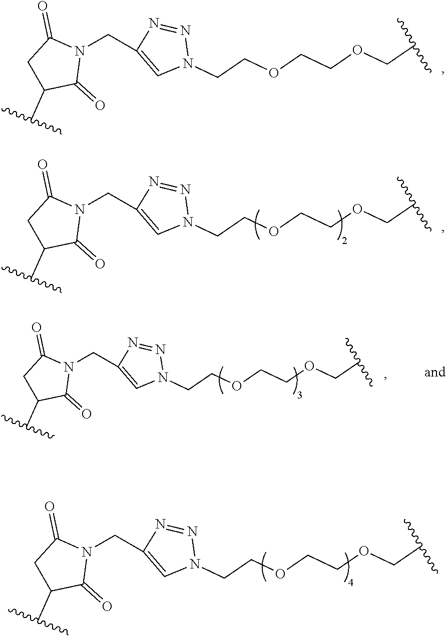

In certain embodiments, in the compound of Formula II, the Linker^{A} is selected from

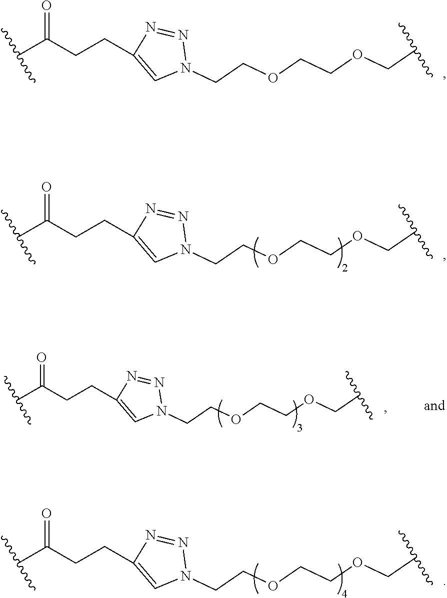

In certain embodiments, in the compound of Formula II, the Linker^{A} is selected from

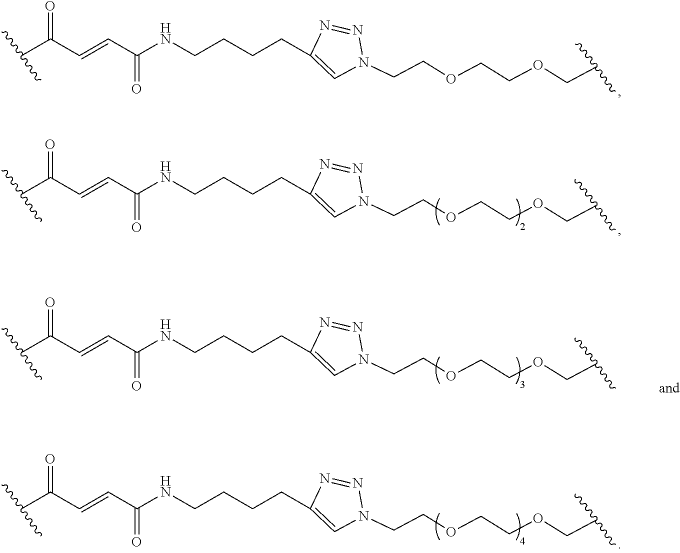

In certain embodiments, in the compound of Formula II, the Linker^{A} is selected from

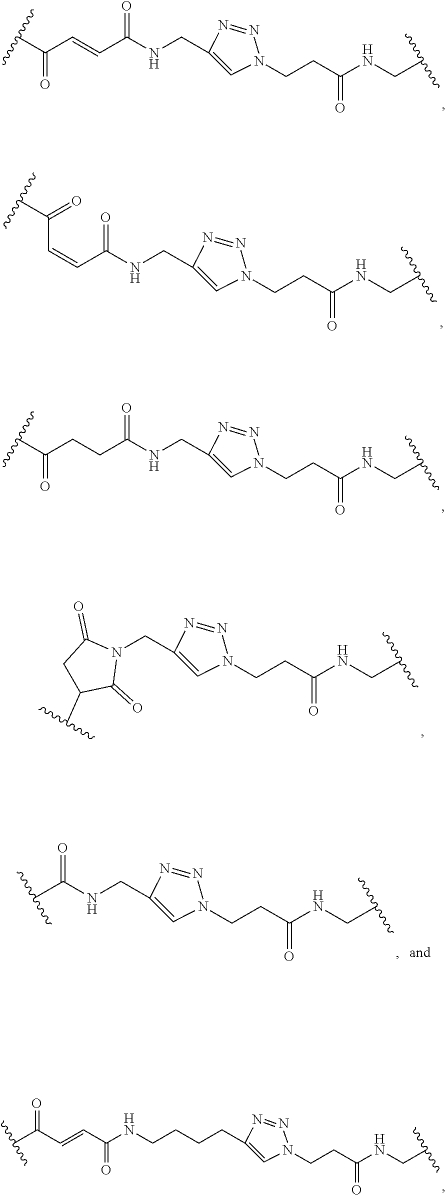 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CNC(*)=O)nn1 ; *CNC(=O)CCn1cc(CCCCNC(=O)/C=C/C(*)=O)nn1]

In certain embodiments, in the compound of Formula II, the Linker^{A} is selected from

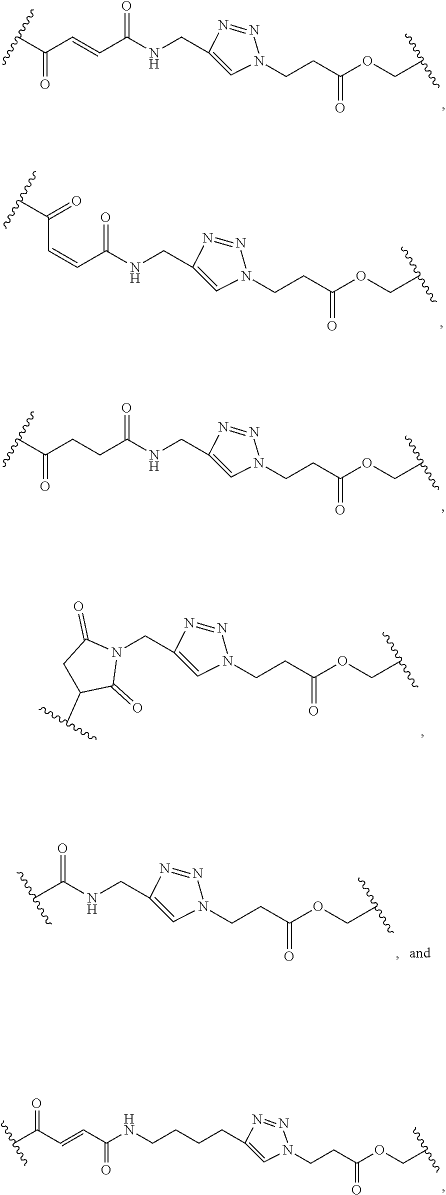 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CNC(*)=O)nn1 ; *COC(=O)CCn1cc(CCCCNC(=O)/C=C/C(*)=O)nn1]

In certain embodiments, in the compound of Formula II, the Linker^{B} is selected from

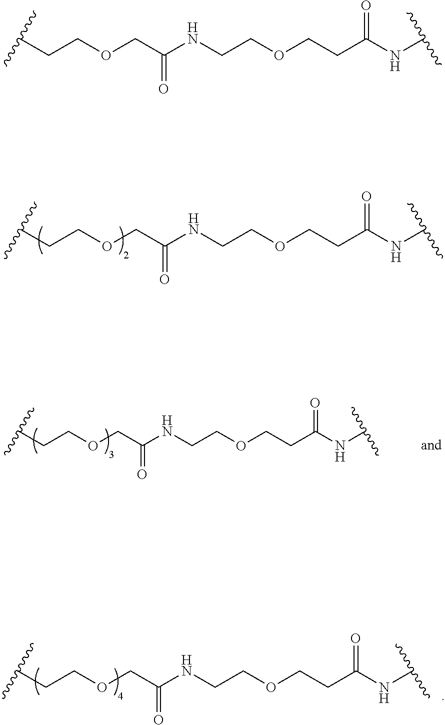

In certain embodiments, in the compound of Formula II, the Linker^{B} is selected from

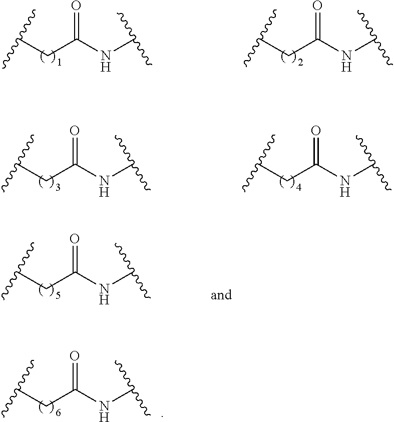

In certain embodiments, in the compound of Formula II, the Linker^{B} is selected from

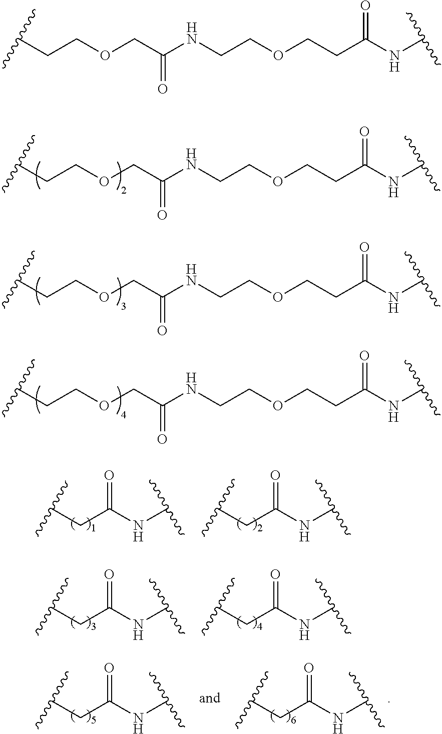

In certain embodiments, in the compound of Formula II, the Linker^{B} is selected from wherein each is optionally substituted with 1, 2, 3, or 4 substituents substituent selected from R²¹.

In certain embodiments, in the compound of Formula II Linker^{B} is selected from:

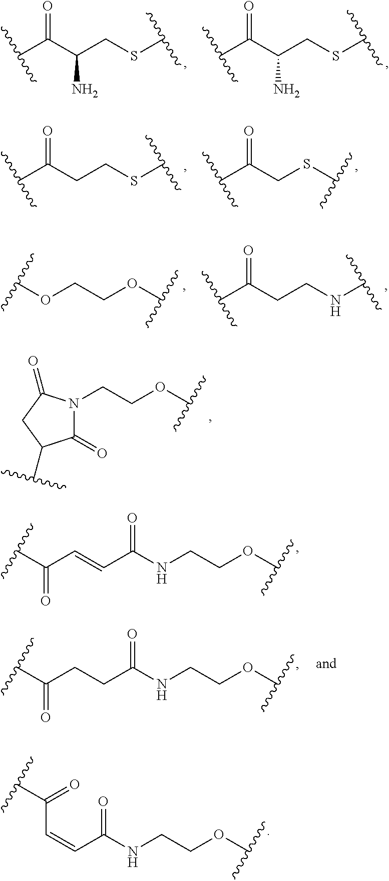

In certain embodiments, in the compound of Formula II, the Linker^{B} is selected from:

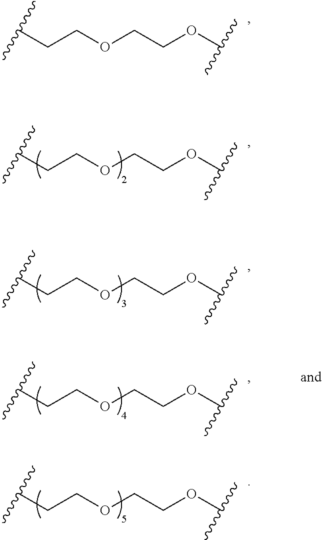

In certain embodiments, in the compound of Formula II, the Linker^{B} is selected from:

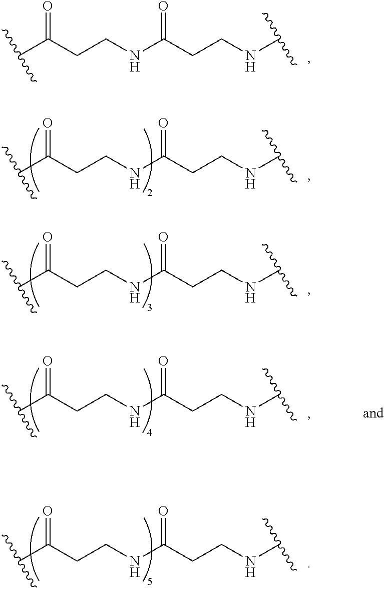

In certain embodiments, in the compound of Formula II, the Linker^{B} is selected from:

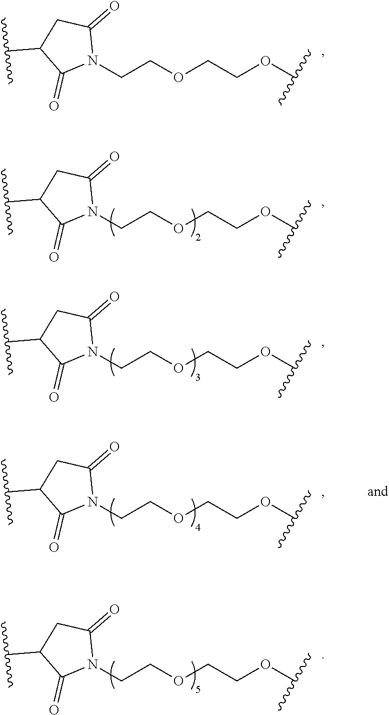

In certain embodiments, in the compound of Formula II, the Linker^{B} is selected from:

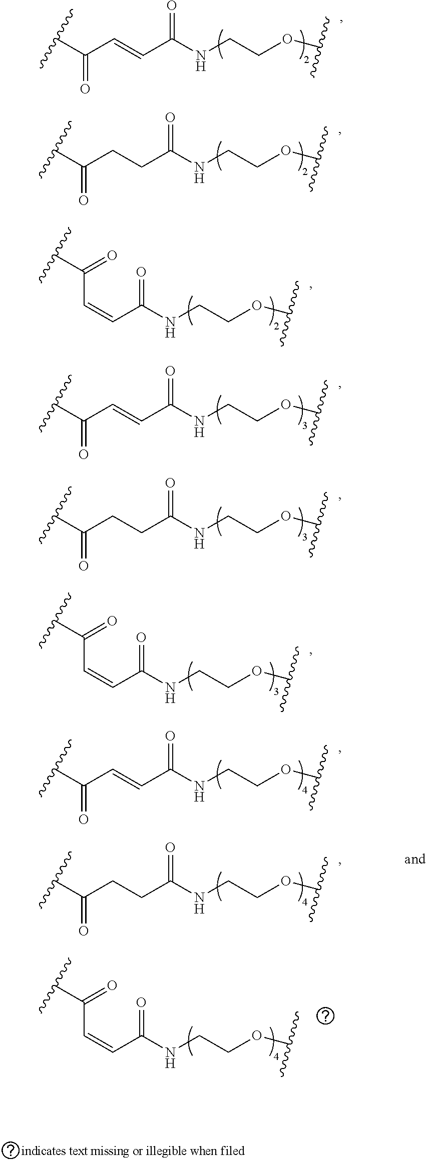

In certain embodiments, in the compound of Formula II, the Linker^{B} is selected from:

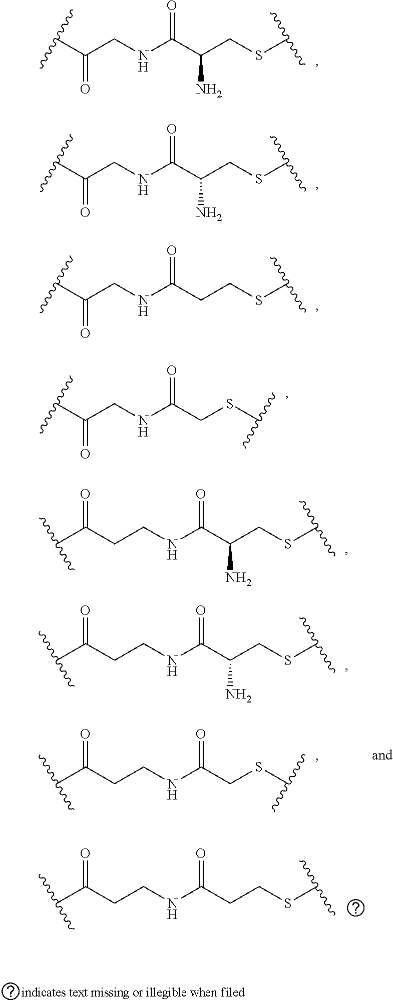

In certain embodiments, in the compound of Formula II, the Linker^{B} is selected from:

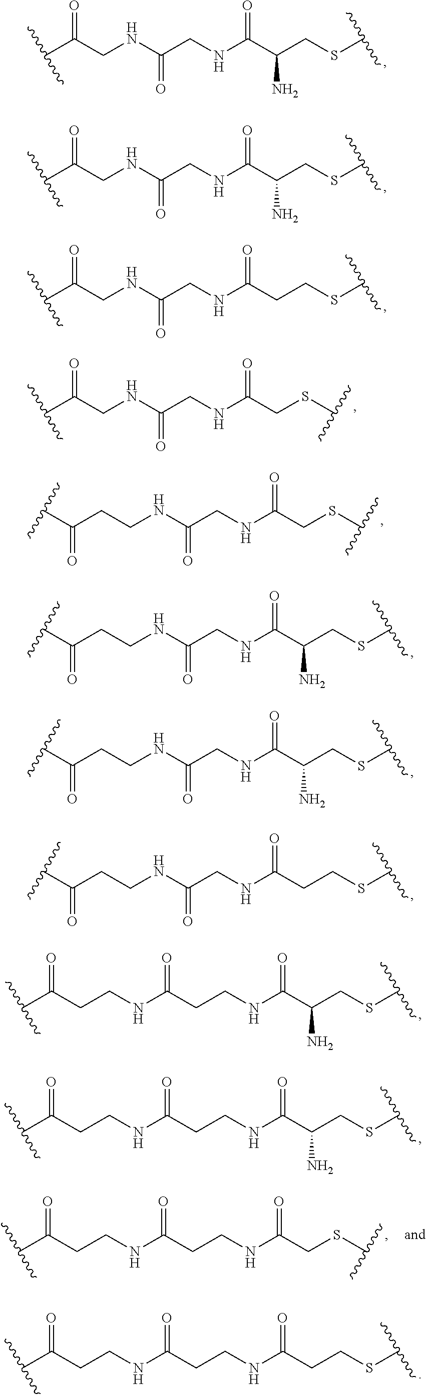

In certain embodiments, in the compound of Formula II, the Linker^{B} is selected from:

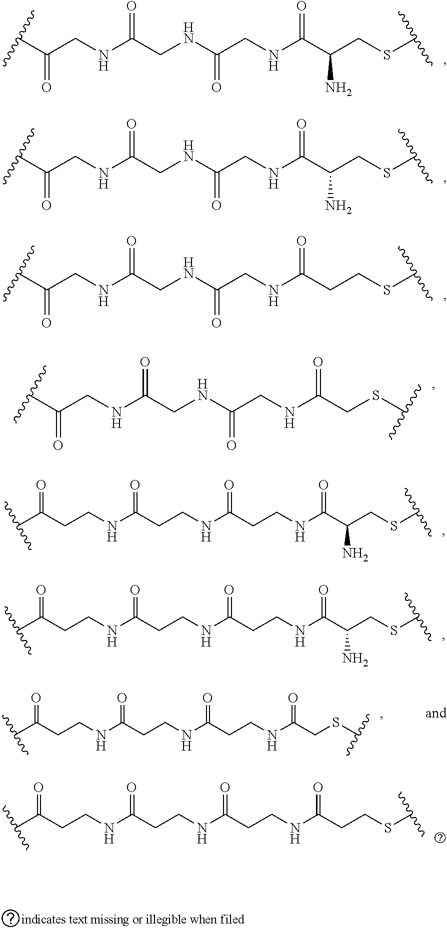

In certain embodiments, in the compound of Formula II, Linker^{B}-Linker^{A} is selected from:

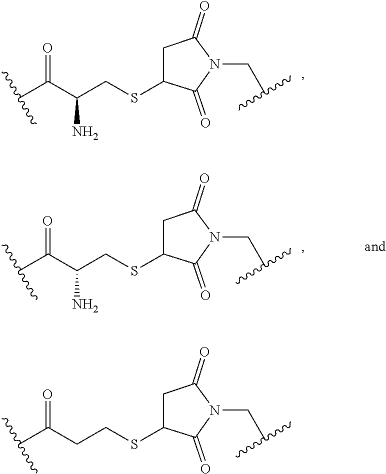 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments, in the compound of Formula II, Linker^{B}-Linker^{A} is selected from:

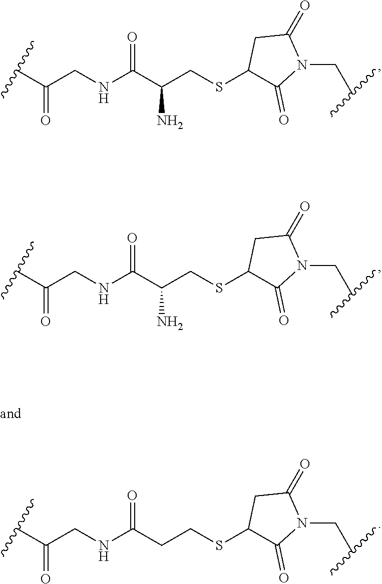 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, in the compound of Formula II, the Linker^{C} is selected from:

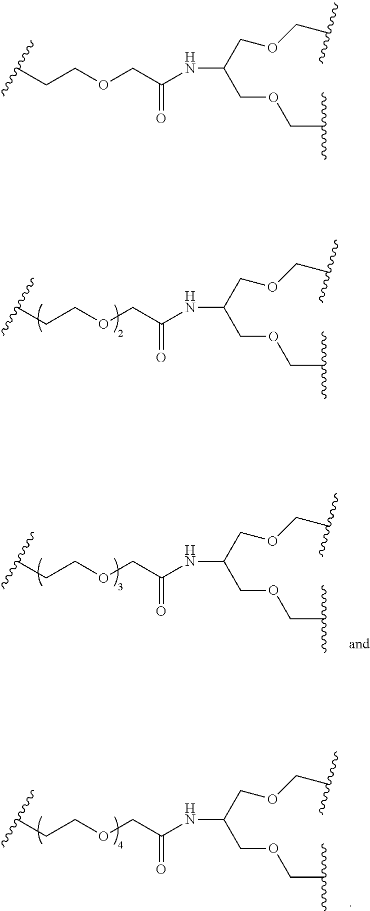

In certain embodiments, in the compound of Formula II, the Linker^{C} is selected from:

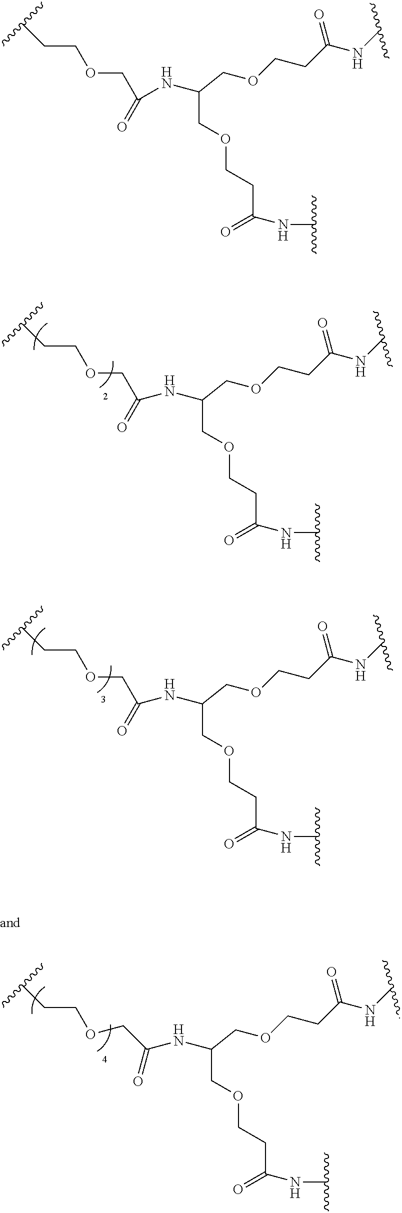

In certain embodiments, in the compound of Formula II, the Linker^{C} is selected from:

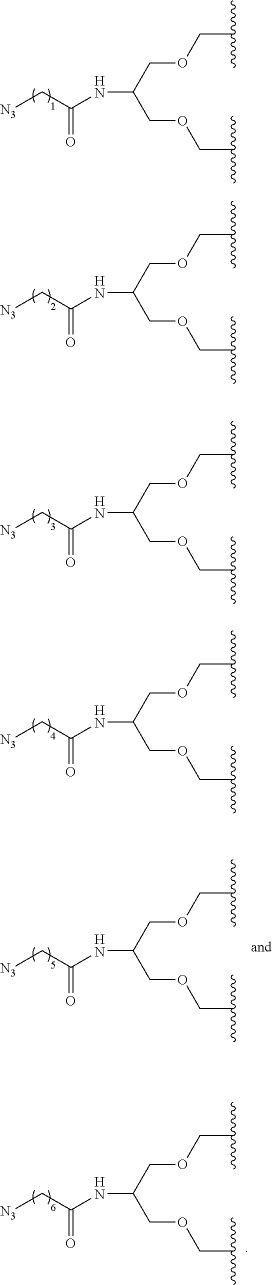

In certain embodiments, in the compound of Formula II, the Linker^{C} is selected from:

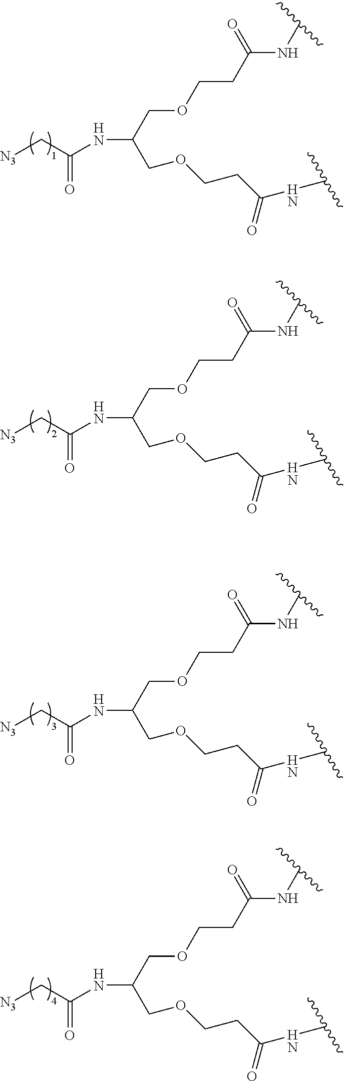

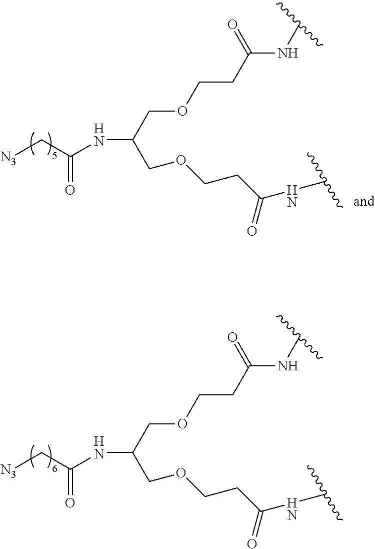

In certain embodiments, in the compound of Formula II, the Linker^{C} is selected from:

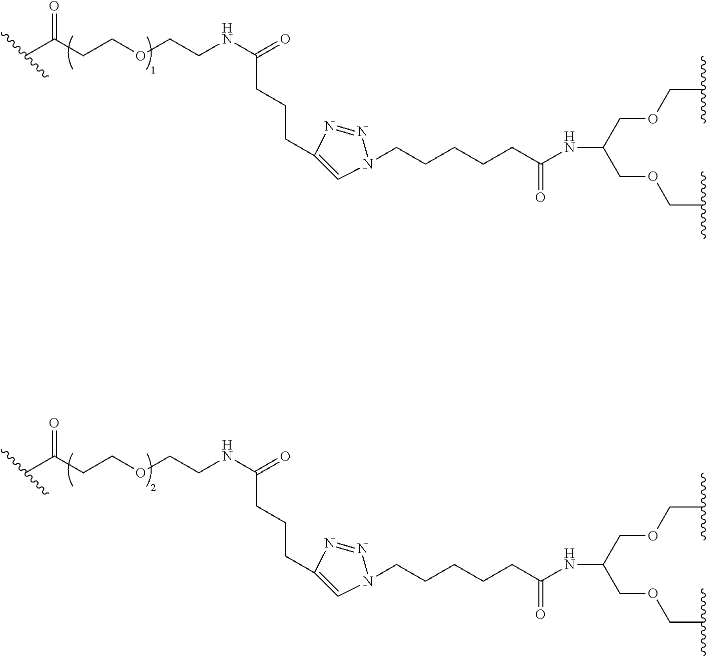

In certain embodiments, in the compound of Formula II, the Linker^{C} is selected from:

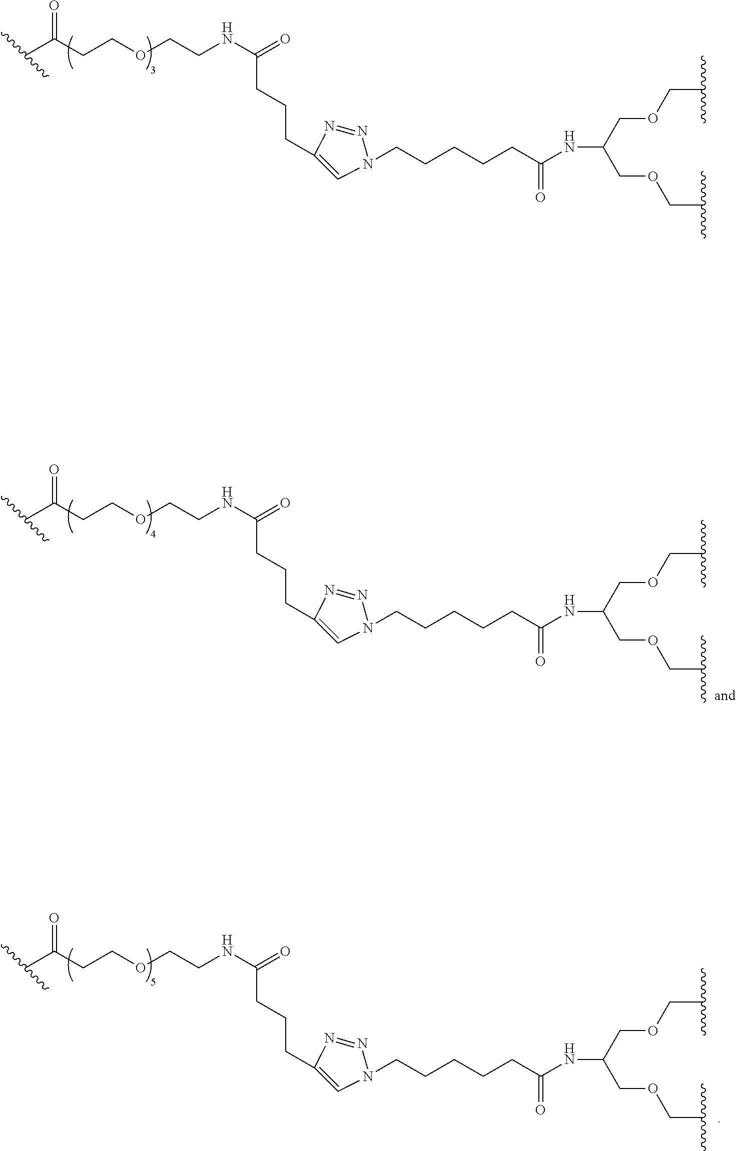

In certain embodiments, in the compound of Formula II, the Linker^{C} is selected from:

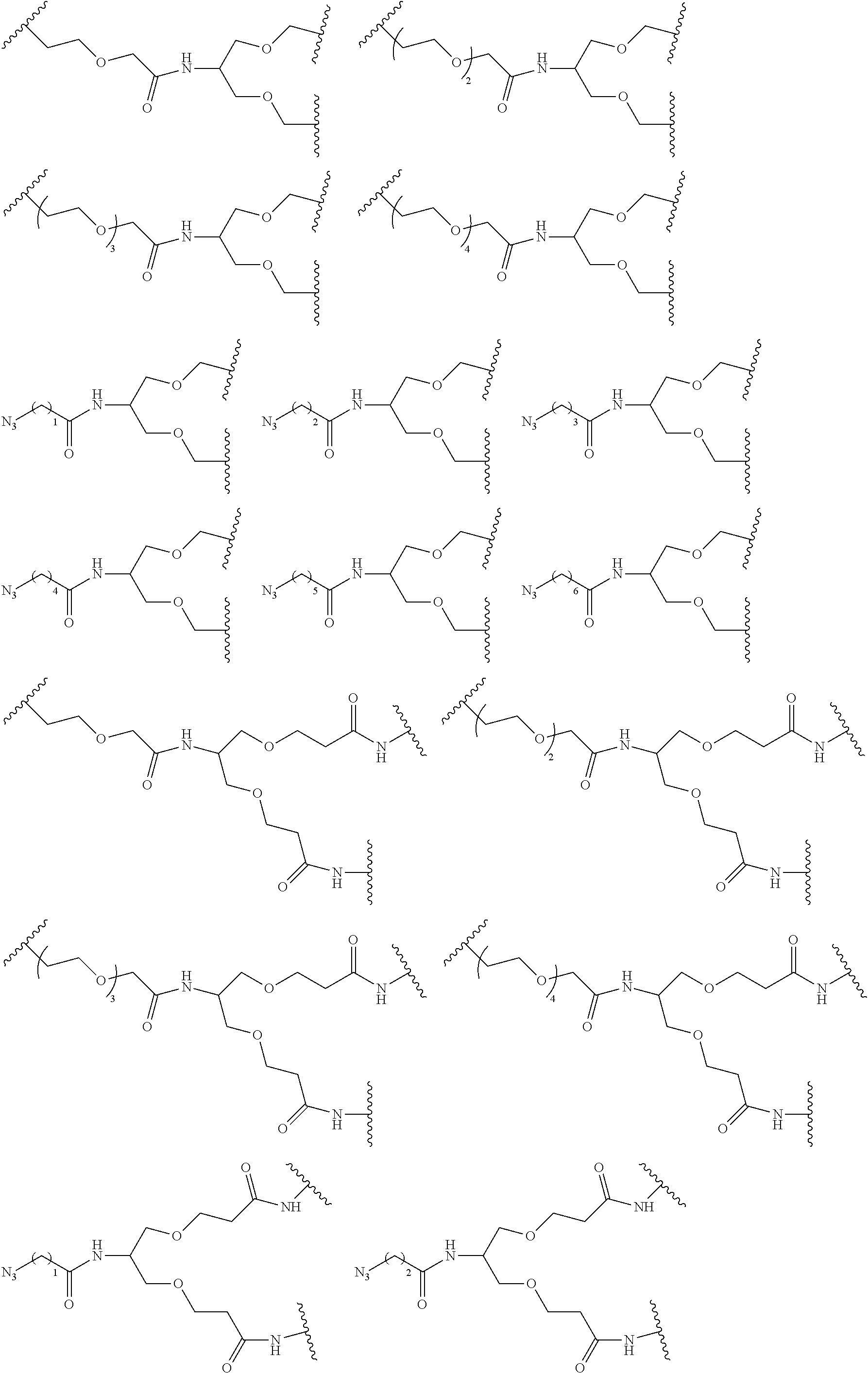

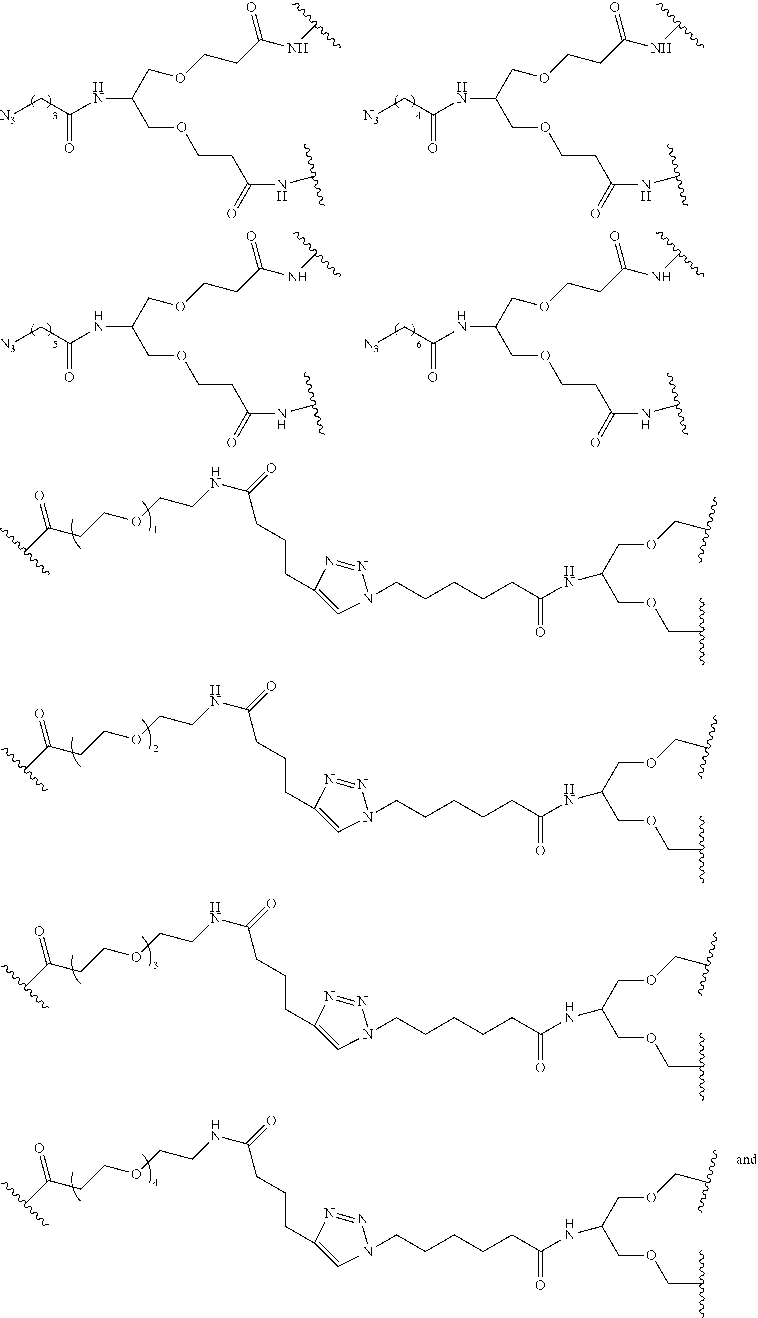

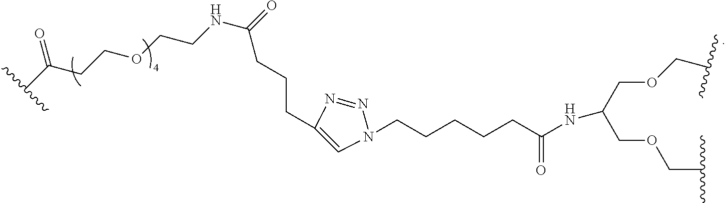

In certain embodiments, in the compound of Formula II, the Linker^{C} is selected from: wherein each is optionally substituted with 1, 2, 3, or 4 substituents substituent selected from R²¹.

In certain embodiments, in the compound of Formula II, the Linker^{C} is selected from:

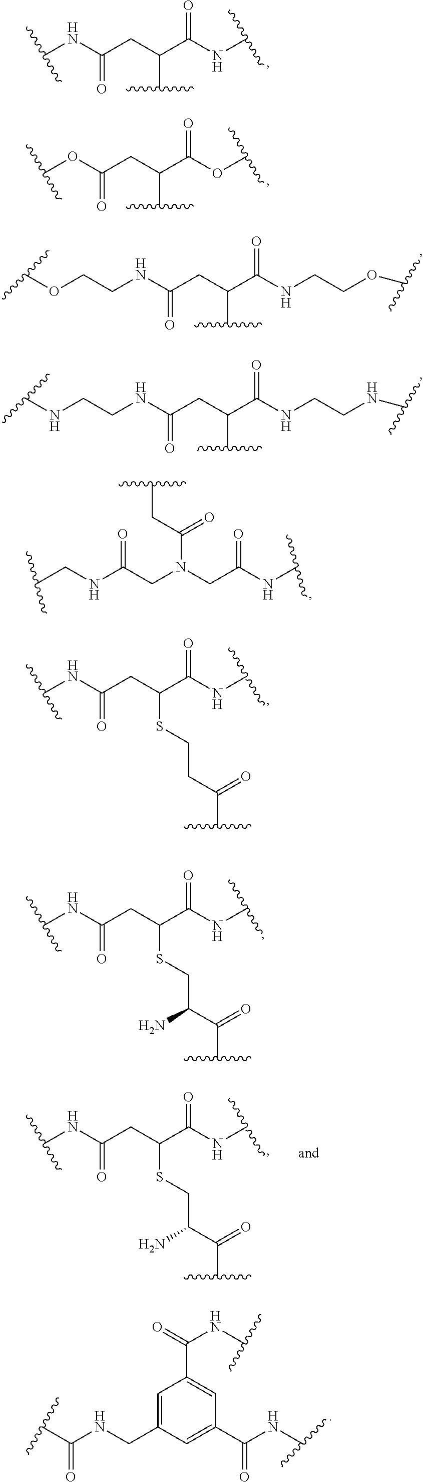

In certain embodiments, in the compound of Formula II, the Linker^{C} is selected from:

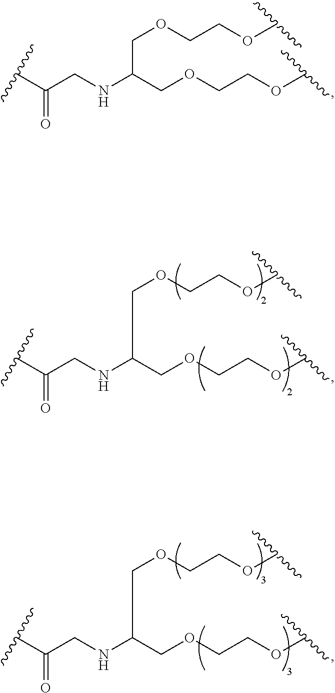

In certain embodiments, in the compound of Formula II, the Linker^{C} is selected from:

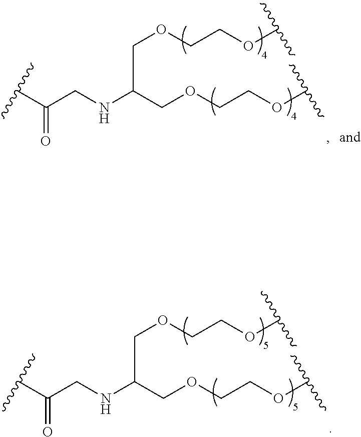

In certain embodiments, in the compound of Formula II, the Linker^{C} is selected from:

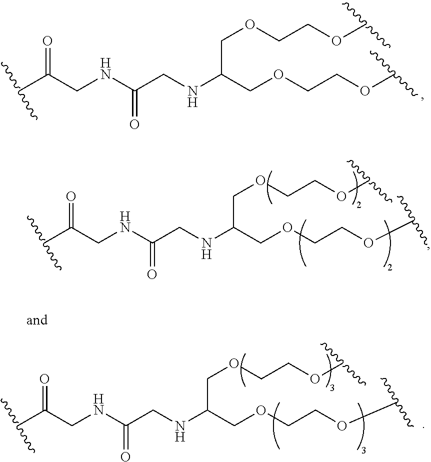

In certain embodiments, in the compound of Formula II, the Linker^{C} is selected from:

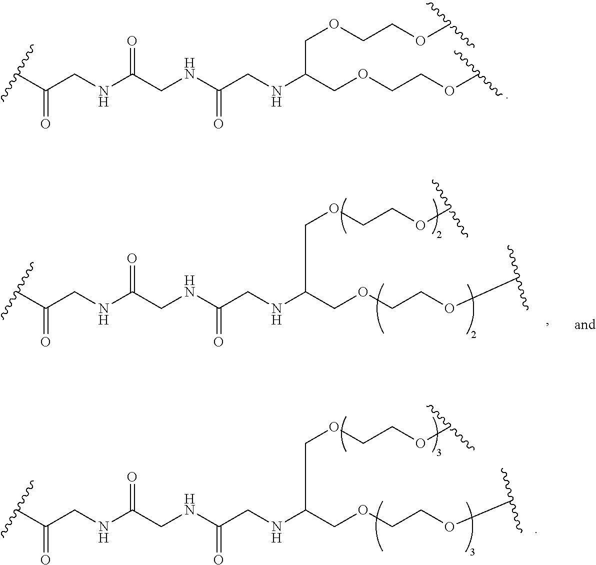

In certain embodiments, in the compound of Formula II, the Linker^{C} is selected from:

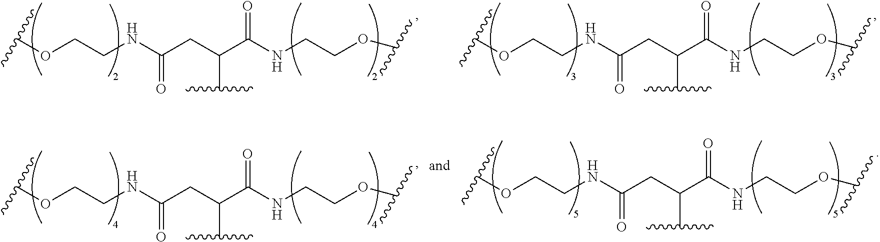

In certain embodiments, in the compound of Formula II, the Linker^{C} is selected from:

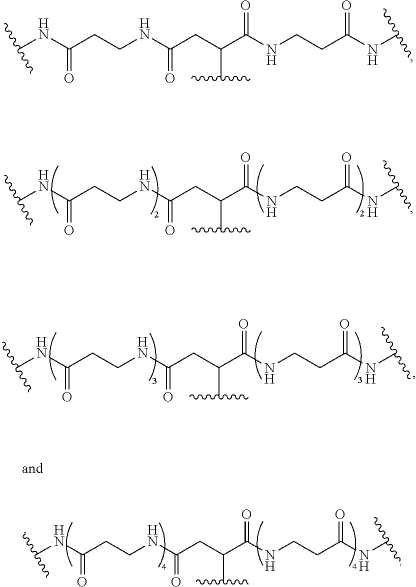

In certain embodiments, in the compound of Formula II, the Linker^{C} is selected from:

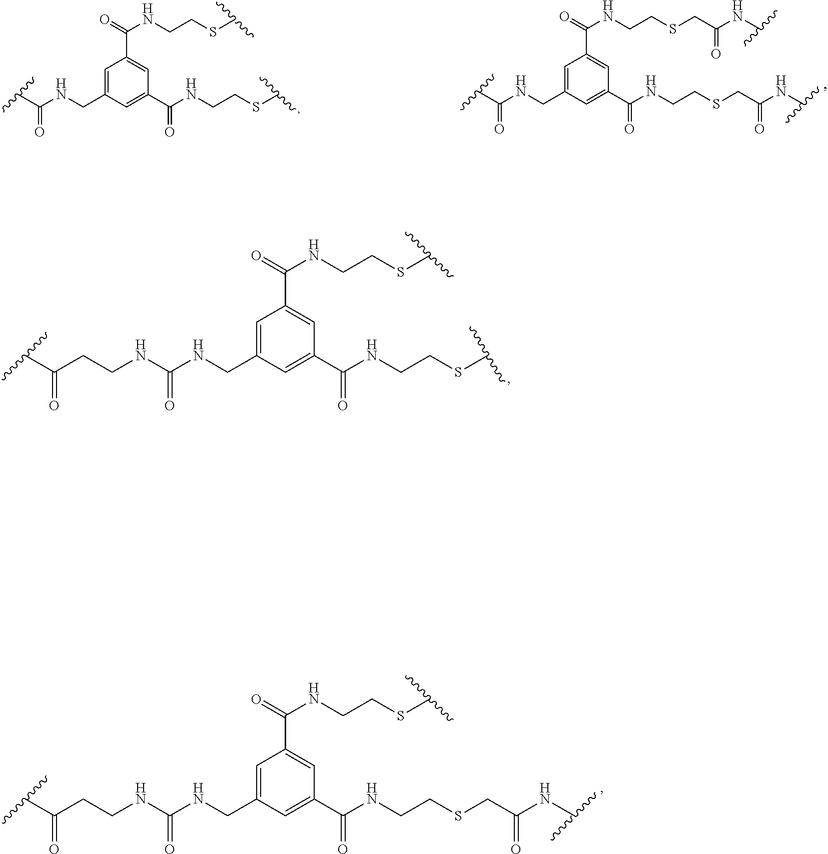 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCS*)c1]

In certain embodiments, in the compound of Formula II, Linker^{C}-(Linker^{A})₂ is selected from:

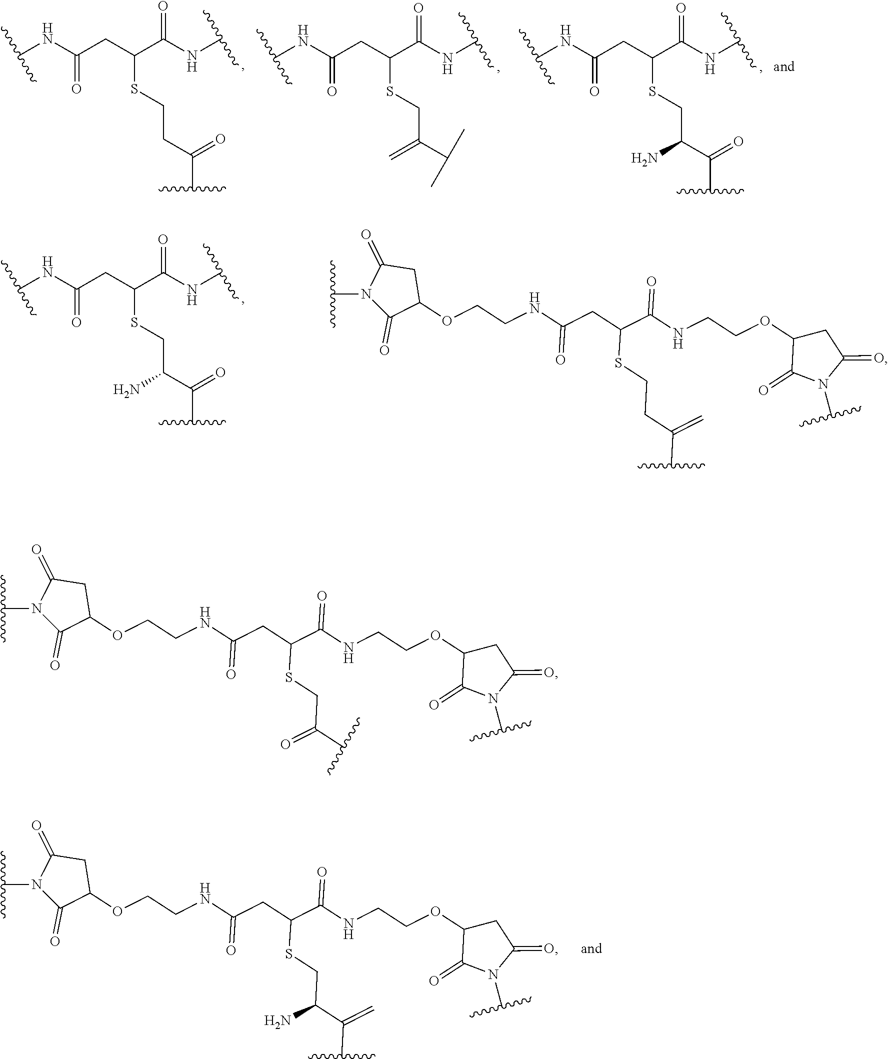

In certain embodiments, in the compound of Formula II, Linker^{C}-(Linker^{A})₂ is selected from:

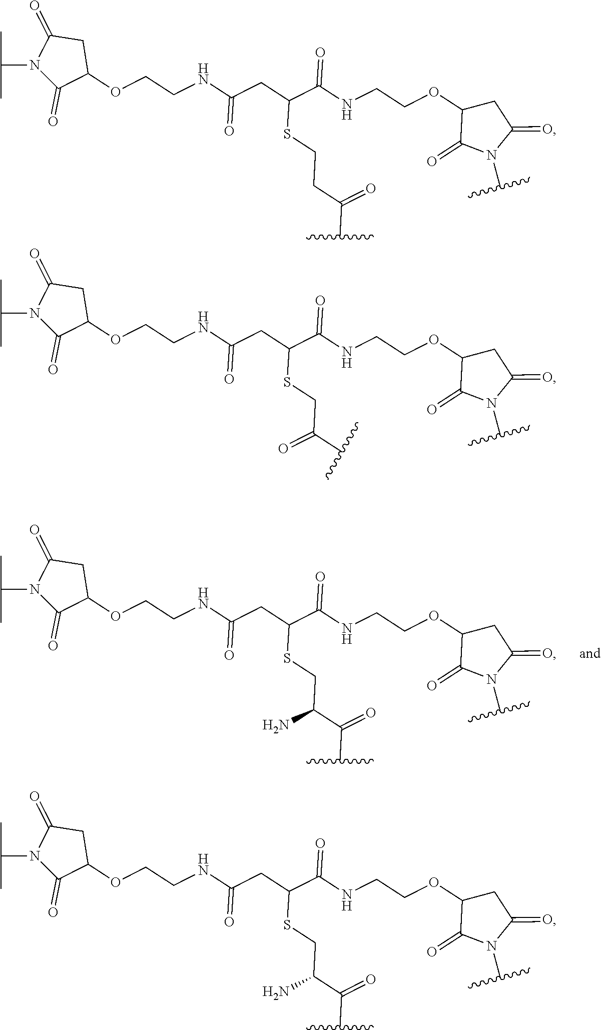 [SMILES: *C(=O)CCSC(CC(=O)NCCOC1CC(=O)N(C(C)C)C1=O)C(=O)NCCOC1CC(=O)N(*)C1=O ; *C(=O)CSC(CC(=O)NCCOC1CC(=O)N(C(C)C)C1=O)C(=O)NCCOC1CC(=O)N(*)C1=O ; *C(=O)[C@@H](N)CSC(CC(=O)NCCOC1CC(=O)N(C(C)C)C1=O)C(=O)NCCOC1CC(=O)N(*)C1=O ; "and" ; *C(=O)[C@H](N)CSC(CC(=O)NCCOC1CC(=O)N(C(C)C)C1=O)C(=O)NCCOC1CC(=O)N(*)C1=O]

In certain embodiments, in the compound of Formula II, Linker^{C}-(Linker^{A})₂ is selected from:

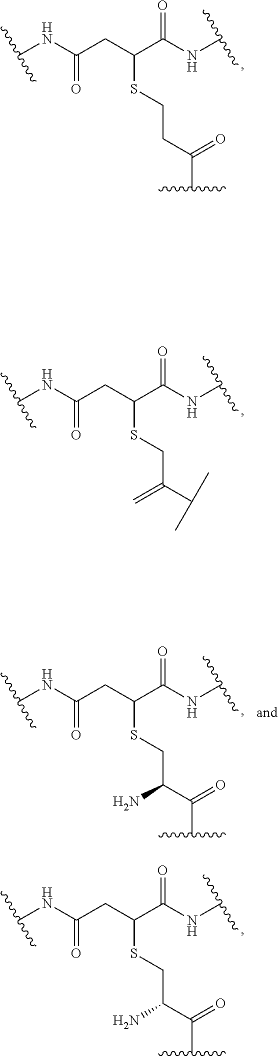

In certain embodiments, in the compound of Formula II, Linker^{C}-(Linker^{A})₂ is selected from:

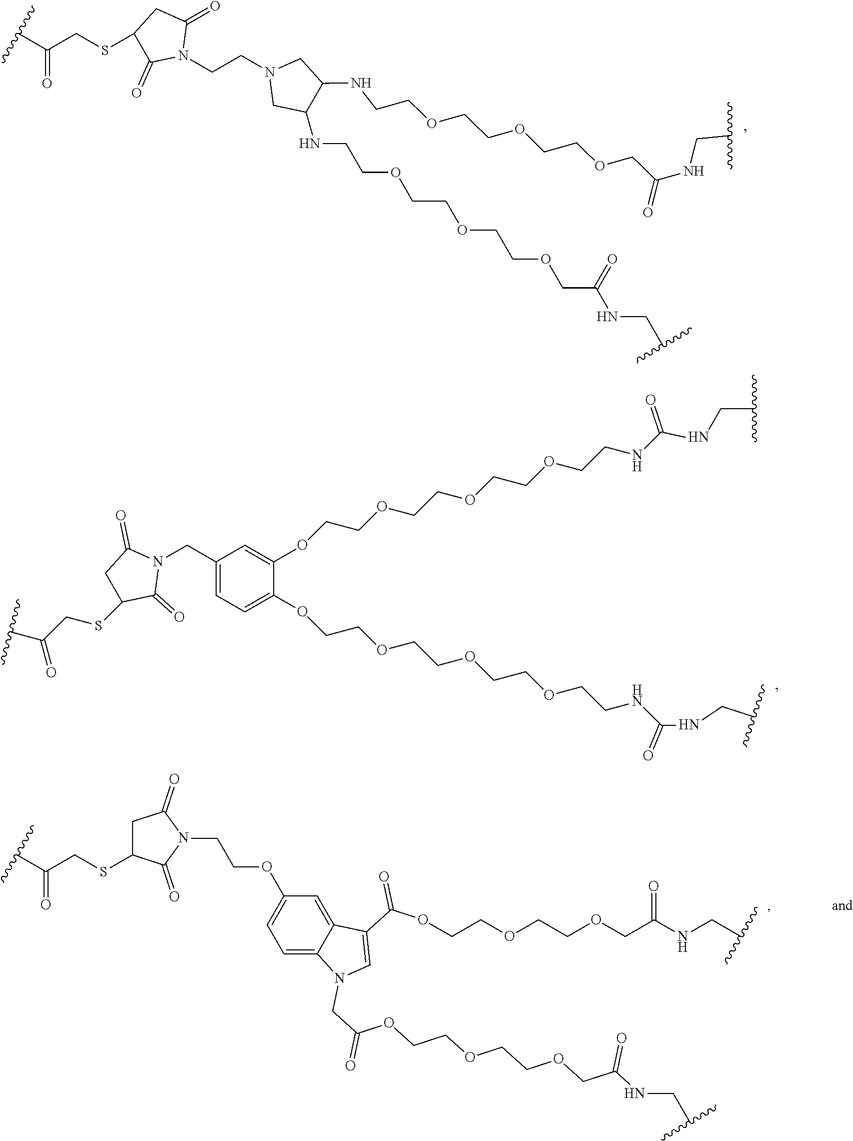 [SMILES: *CNC(=O)COCCOCCOCCNC1CN(CCN2C(=O)CC(SCC(*)=O)C2=O)CC1NCCOCCOCCOCC(=O)NC* ; *CNC(=O)NCCOCCOCCOCCOc1ccc(CN2C(=O)CC(SCC(*)=O)C2=O)cc1OCCOCCOCCOCCNC(=O)NC* ; *CNC(=O)COCCOCCOC(=O)Cn1cc(C(=O)OCCOCCOCC(=O)NC*)c2cc(OCCN3C(=O)CC(SCC(*)=O)C3=O)ccc21]

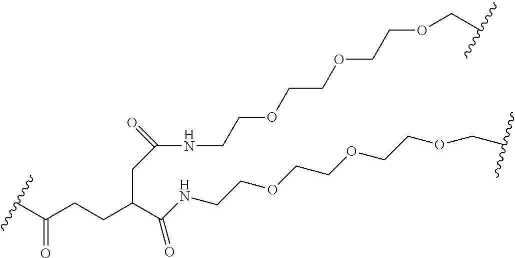 [SMILES: *COCCOCCOCCNC(=O)CC(CCC(*)=O)C(=O)NCCOCCOCCOC*]

In certain embodiments, in the compound of Formula II, Linker^{D} is selected from:

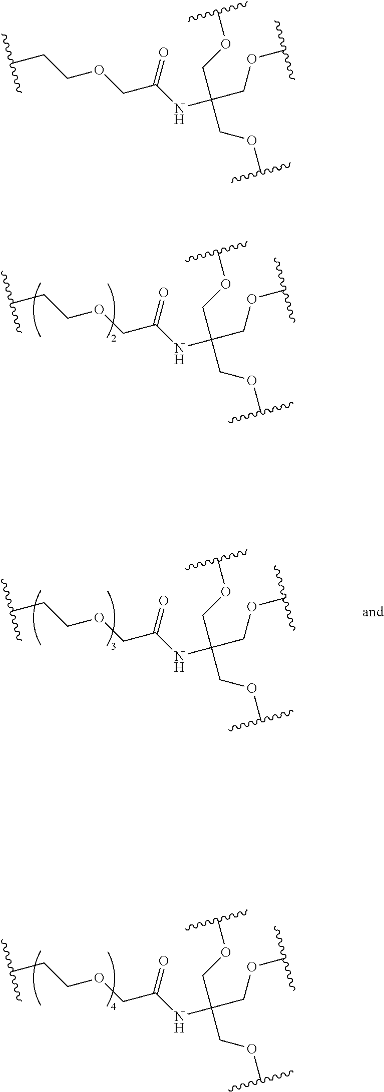

In certain embodiments, in the compound of Formula II, Linker^{D} is selected from:

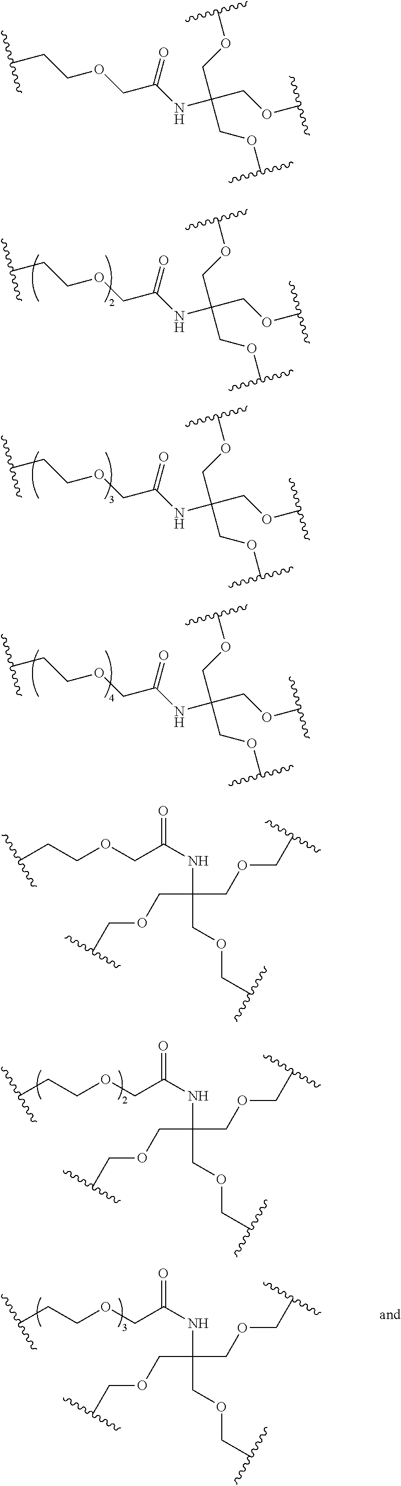

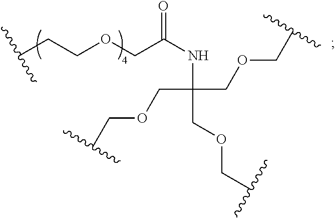

wherein each is optionally substituted with 1, 2, 3, or 4 substituents are selected from R²¹.

In certain embodiments, in the compound of Formula II, Linker^{B}-(Linker^{A}) is selected from

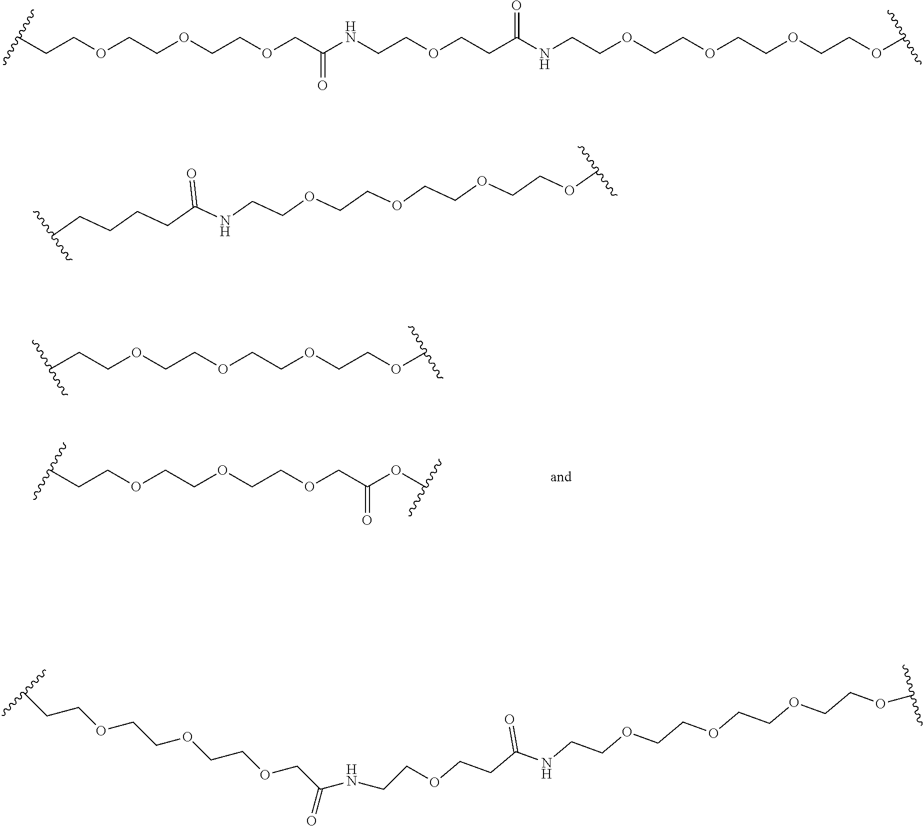 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, in the compound of Formula II, Linker^{C}-(Linker^{A}) is selected from

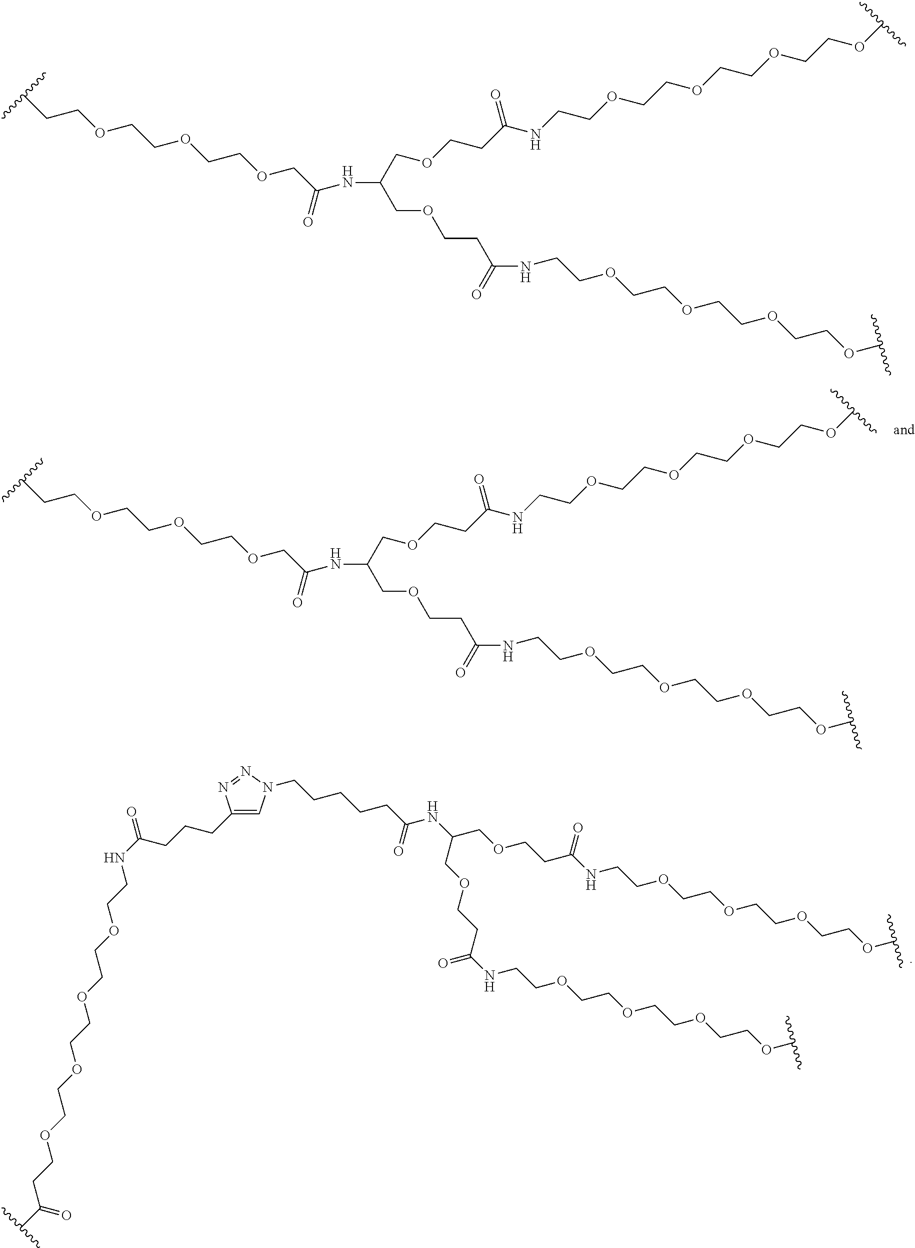 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO* ; *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, in the compound of Formula II, Linker^{D}-(Linker^{A}) is selected from

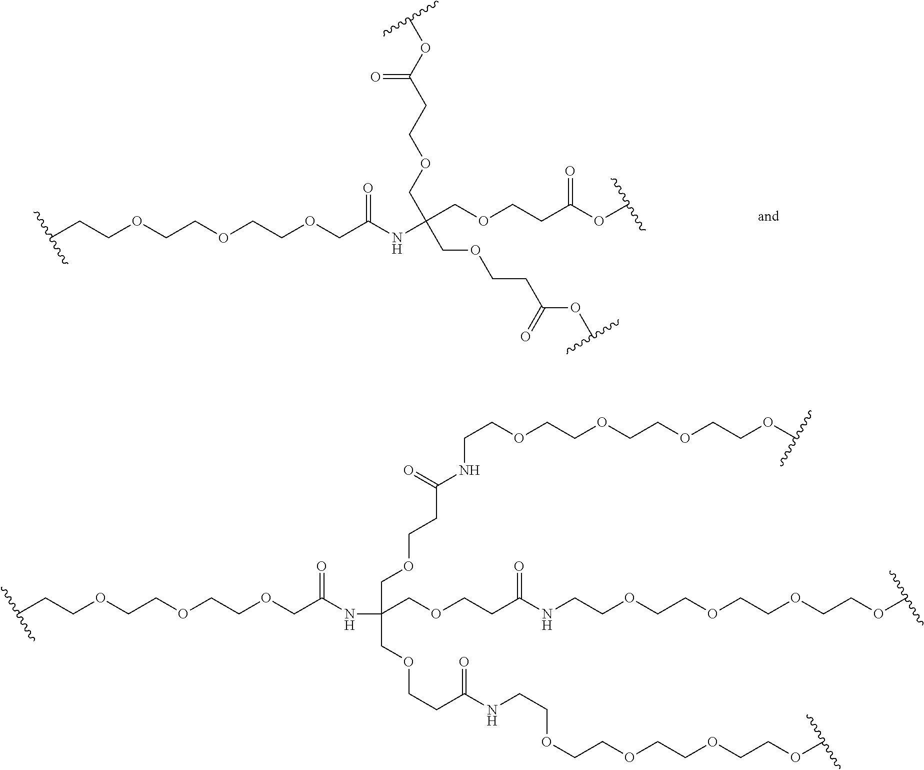 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

In various embodiments, R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷, C(O)R³, S(O)R³, C(S)R³, and S(O)₂R³.

In various embodiments, in the compound of Formula II, R⁵ is independently selected from hydrogen, heteroalkyl,

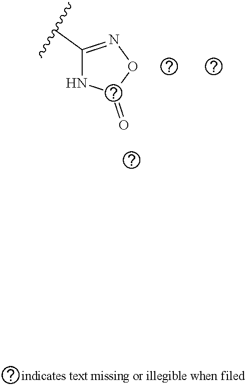 [SMILES: *c1noc(=O)[nH]1]

C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³ C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents.

In various embodiments, in the compound of Formula II, R⁶ and R⁷ are independently selected at each occurrence from hydrogen, heteroalkyl, alkyl, arylalkyl, heteroaryl alkyl, alkenyl, alkynyl, and, haloalkyl, heteroaryl, heterocycle, -alkyl-OR⁸, -alkyl-NR⁸R⁹, C(O)R³, S(O)R³, C(S)R³, and S(O)₂R³.

In various embodiments, in the compound of Formula II, R⁸ and R⁹ are independently selected at each occurrence from hydrogen, heteroalkyl, alkyl, arylalkyl, heteroarylalkyl, alkenyl, alkynyl, aryl, heteroaryl, and heterocycle.

In various embodiments, the compound of Formula II has the structure of Formula II-A. In various embodiments, in the compound of Formula II, [Ab] is as defined herein.

A compound of Formula II-A, having the structure:

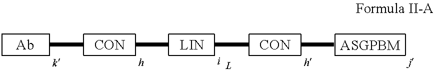

wherein:

[Ab] is an antibody that binds to an extracellular protein;

[ASGPBM] is an asialoglycoprotein receptor binding moiety having the structure selected from

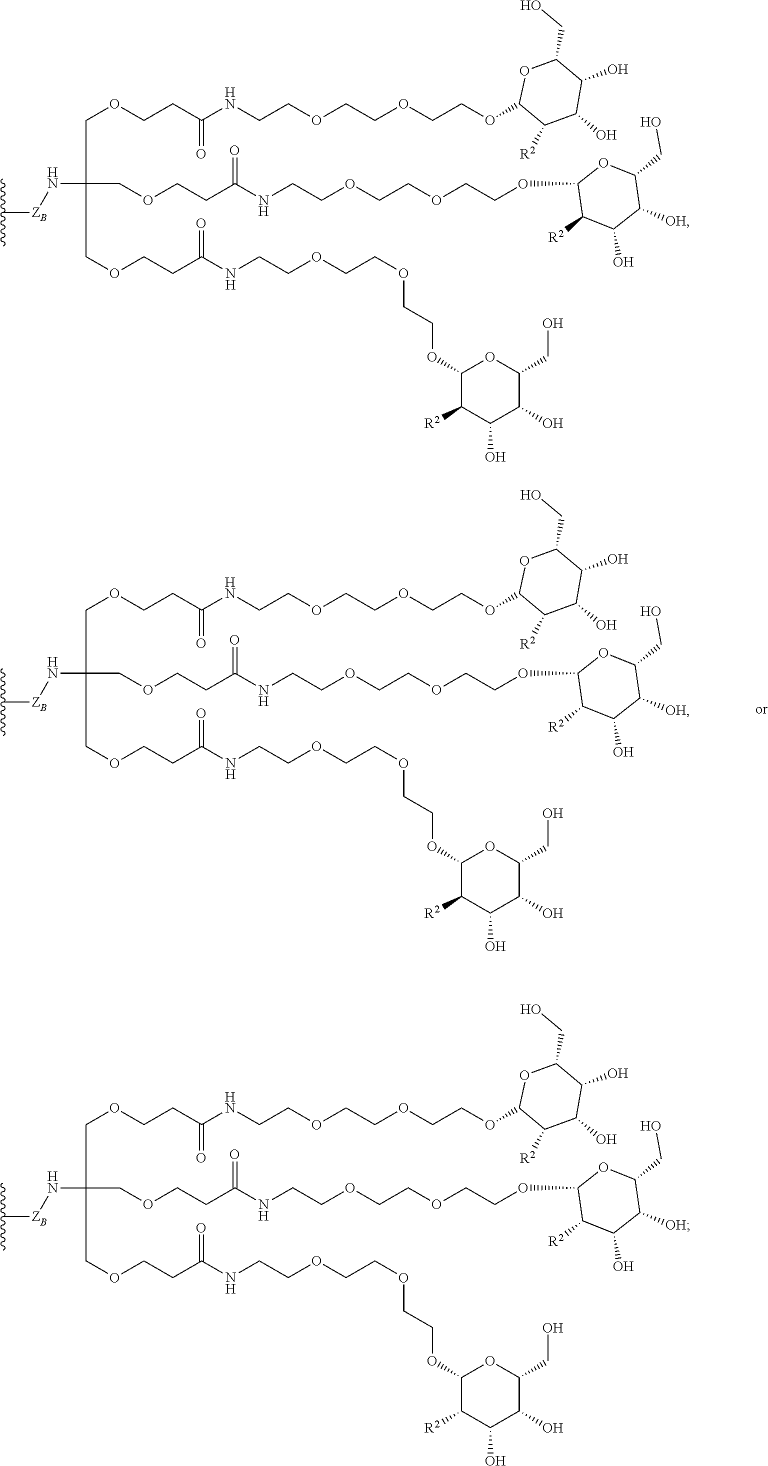

each [CON] is an optional connector chemical moiety which, when present, connects the [LIN] to [CPBM] or to [ASGPBM];

[LIN] is [LINKER] or [LINKER-2], each of which is a chemical moiety having a valency from 1 to 15, which covalently attaches to one or more [ASGPBM] or [CPBM] groups, optionally through a [CON], wherein the [LIN] optionally itself contains one or more [CON] groups;
Z_{B} is absent, (CH₂)_{IM}, C(O)—(CH₂)_{IM}—, or C(O)—(CH₂)_{IM}—NR_{M};
R_{M} is H or a C₁-C₃ alkyl group optionally substituted with one or two hydroxyl groups;

R₂ is

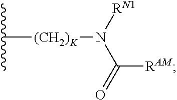

wherein R^{AM} is H, C₁-C₄ alkyl optionally substituted with up to 3 halo groups and one or two hydroxyl groups, —(CH₂)_{K}COOH, —(CH₂)_{K}C(O)O—(C₁-C₄ alkyl) optionally substituted with 1-3 halo groups, —O—C(O)—(C₁-C₄ alkyl) optionally substituted with 1-3 halo groups, —C(O)—(C₁-C₄ alkyl) optionally substituted with 1-3 halo groups, or —(CH₂)_{K}—NR^{N3}R^{N4}, or

R₂ is

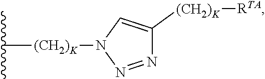

wherein
R^{TA} is H, CN, NR^{N1}R^{N2}, —(CH₂)_{K}OH, —(CH₂)_{K}O(C₁-C₄ alkyl) optionally substituted with 1-3 halo groups, C₁-C₄ alkyl optionally substituted with 1-3 halo groups, —(CH₂)_{K}COOH, —(CH₂)_{K}C(O)O—(C₁-C₄ alkyl) optionally substituted with 1-3 halo groups, —O—C(O)—(C₁-C₄ alkyl) optionally substituted with 1-3 halo groups, or —C(O)—(C₁-C₄ alkyl) optionally substituted with 1-3 halo groups, or
R^{TA} is a C₃-C₁₀ aryl or a three- to ten-membered heteroaryl group containing up to 5 heteroaryl atoms, each of the aryl or heteroaryl groups being optionally substituted with up to three CN, NR^{N1}R^{N2}, —(CH₂)_{K}OH, —(CH₂)_{K}O(C₁-C₄ alkyl) optionally substituted with 1-3 halo groups, C₁-C₃ alkyl optionally substituted with 1-3 halo groups or 1-2 hydroxy groups, —O—(C₁-C₃-alkyl) optionally substituted from 1-3 halo groups, —(CH₂)_{K}COOH, —(CH₂)_{K}C(O)O—(C₁-C₄ alkyl) optionally substituted with 1-3 halo groups, O—C(O)—(C₁-C₄ alkyl) optionally substituted with 1-3 halo groups, or —(CH₂)_{K}C(O)—(C₁-C₄ alkyl) optionally substituted with 1-3 halo groups, or
R^{TA} is

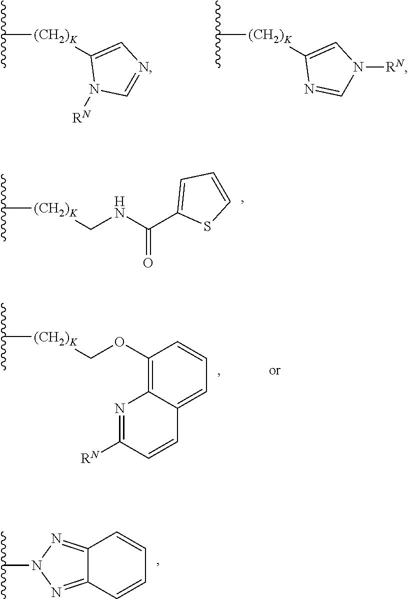

optionally substituted with up to three C₁-C₃ alkyl groups which are optionally substituted with up to three halo groups; or
R^{TA} is

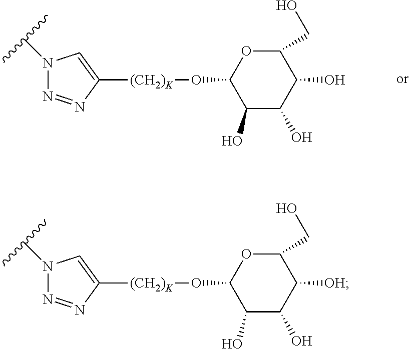

R^{N}, R^{N1}, R^{N2}, R^{N3}, R^{N4} are each independently H or C₁-C₃ alkyl optionally substituted with one to three halo groups or one or two hydroxyl groups and each —(CH₂)_{K} group is optionally substituted with 1-4 C₁-C₃ alkyl groups which are optionally substituted with 1-3 fluoro groups or 1-2 hydroxyl groups;

IM is independently at each occurrence an integer from 0 to 6;

K is independently at each occurrence an integer from 0 to 4;

k′ is an integer ranging from 1 to 15;

j′ is an integer ranging from 1 to 15;

h and h′ are each independently an integer ranging from 0 to 15;

i_{L} is 0 to 15;

with the proviso that at least one of h, h′, and i_{L} is at least 1, or a salt, stereoisomer, or solvate thereof.
In various embodiments, in the compound of Formula II-A, R₂ is —NC(═O)CH₃.

D. Other-Based ASGPR-Binding Moieties

In some embodiments, the ASGPR binding moieties can be any of the moieties described in: Reshitko, G. S., et al., “Synthesis and Evaluation of New Trivalent Ligands for Hepatocyte Targeting via the Asialoglycoprotein Receptor,” Bioconjugate Chem, doi: 10.1021/acs.bioconjchem.0c00202; Majouga, A. G., et al., “Identification of Novel Small-Molecule ASGP-R Ligands,” Current Drug Delivery, 2016, 13, 1303-1312, doi: 10.2174/1567201813666160719144651; Olshanova, A. S., et al., “Synthesis of a new betulinic acid glycoconjugate with N-acetyl-D-galactosamine for the targeted delivery to hepatocellular carcinoma cells,” Russian Chemical Bulletin, International Edition, Vol. 69, No. 1, pp. 158-163, January 2020; Yamansarov, E. Yu., et al., “New ASGPR-targeted ligands based on glycoconjugated natural triterpenoids,” Russian Chemical Bulletin, International Edition, Vol. 68, No. 12, pp. 2331-2338, December 2019; Congdon, M. D., et al., “Enhanced Binding and Reduced Immunogenicity of Glycoconjugates Prepared via Solid-State Photoactivation of Aliphatic Diazirine Carbohydrates,” Bioconjugate Chem, doi: 10.1021/acs.bioconjchem.0c00555; and Dhawan, V., et al., “Polysaccharide conjugates surpass monosaccharide ligands in hepatospecific targeting—Synthesis and comparative in silico and in vitro assessment,” Carbohydrate Research 509 (2021) 108417, doi: 10.1016/j.carres.2021.108417. The following ASGPR binding moieties are illustrative and not intended to be limiting.

1. GalNAc-Tyrosine Based Moieties

In some embodiments, the ASGPR binding moiety can be a moiety having the structure of M1, M2, M3, or M4, or a combination thereof. In the structures of M1, M2, M3, and M4, X is independently at each occurrence O, NH, or S. In various embodiments, compounds of Formula I or Formula II can have one, two, or three ASGPR binding moieties with the structure of M1, M2, M3, or M4.

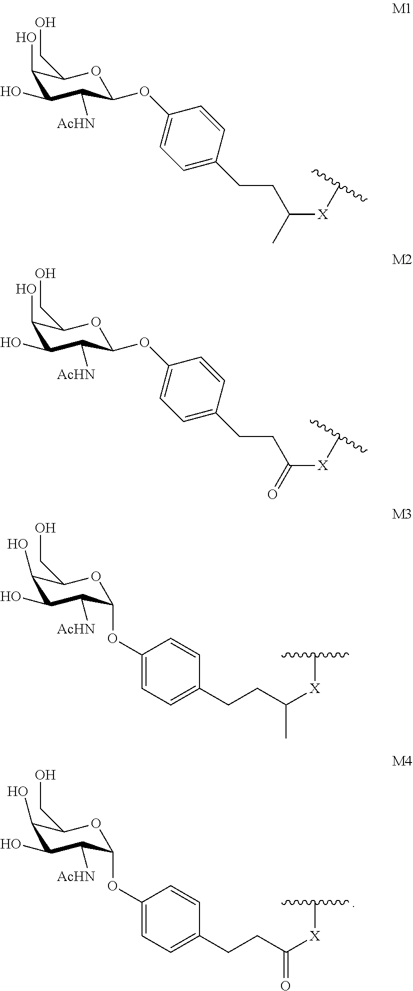

In various embodiments, ASGPR binding moieties M1 to M4 can be conjugated to any suitable [CON], [Linker], or [Linker-2] as described herein and in Congdon, M. D., et al., “Enhanced Binding and Reduced Immunogenicity of Glycoconjugates Prepared via Solid-State Photoactivation of Aliphatic Diazirine Carbohydrates,” Bioconjugate Chem, doi: 10.1021/acs.bioconjchem.0c00555.

2. Trivalent Triazole-Based Moieties

In some embodiments, the ASGPR binding moiety can be a moiety having the structure of M5:

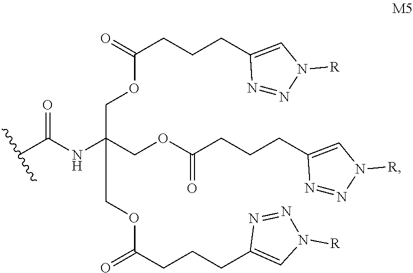

In the structures M5, each R is independently at each occurrence R₁ or R₂,

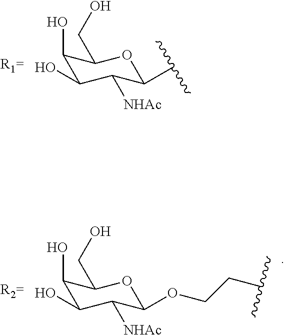 [SMILES: *C1OC(CO)C(O)C(O)C1NC(C)=O ; *CCOC1OC(CO)C(O)C(O)C1NC(C)=O]

In various embodiments, compounds of Formula I or Formula II contain an ASGPR binding moiety with the structure of M5. In various embodiments, each R in M5 is R₁. In various embodiments, each R in M5 is R₂.

In various embodiments, ASGPR binding moiety M5 can be conjugated/bonded to any suitable [CON], [Linker], or [Linker-2] as described herein and in Reshitko, G. S., et al., “Synthesis and Evaluation of New Trivalent Ligands for Hepatocyte Targeting via the Asialoglycoprotein Receptor,” Bioconjugate Chem, doi: 10.1021/acs.bioconjchem.0c00202.

3. Galactose- and Agarose-Derived Behenic Acid Ester Moieties

In various embodiments, the ASGPR binding moiety can be the galactose behenic acid ester-derived moiety M7:

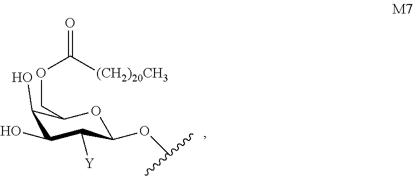

In the structure M7, Y is OH or NHAc.

In various embodiments, the ASGPR binding moiety can be the agarose behenic acid ester-derived moiety M8:

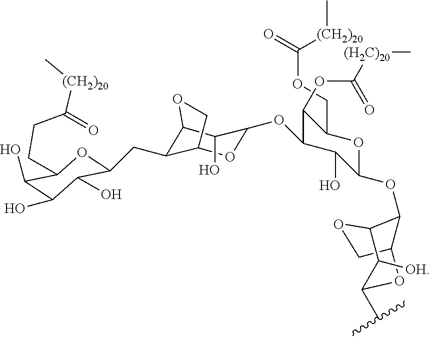

In various embodiments, ASGPR binding moieties M7 and M8 can be conjugated to any suitable [CON], [Linker], or [Linker-2] as described herein and in Dhawan, V., et al., “Polysaccharide conjugates surpass monosaccharide ligands in hepatospecific targeting—Synthesis and comparative in silico and in vitro assessment,” Carbohydrate Research 509 (2021) 108417, doi: 10.1016/j.carres.2021.108417.

4. Other Small Molecule ASGPR Binding Moieties

In various embodiments, the ASGPR binding moiety can be any of the compounds 2-18 below:

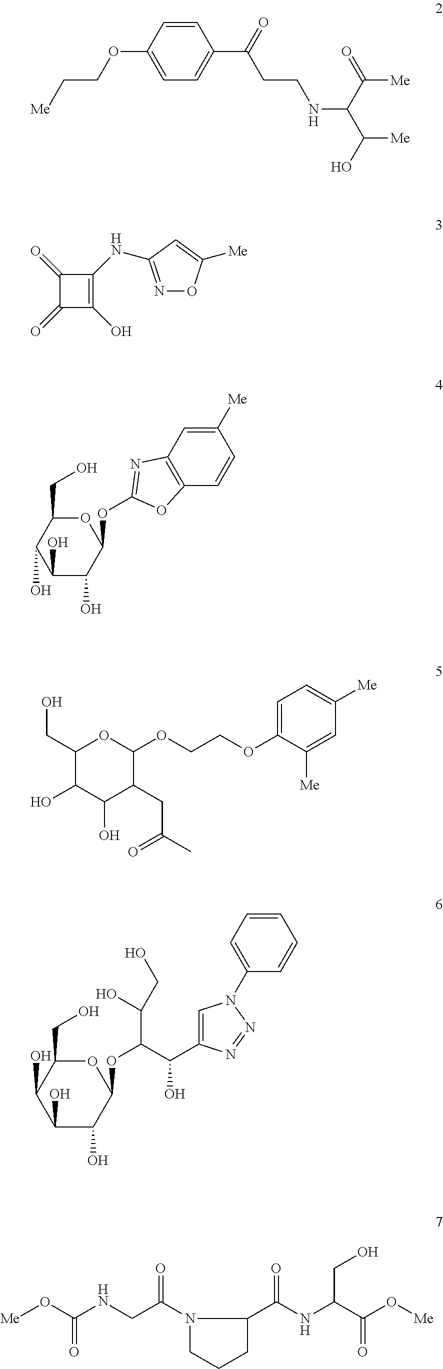

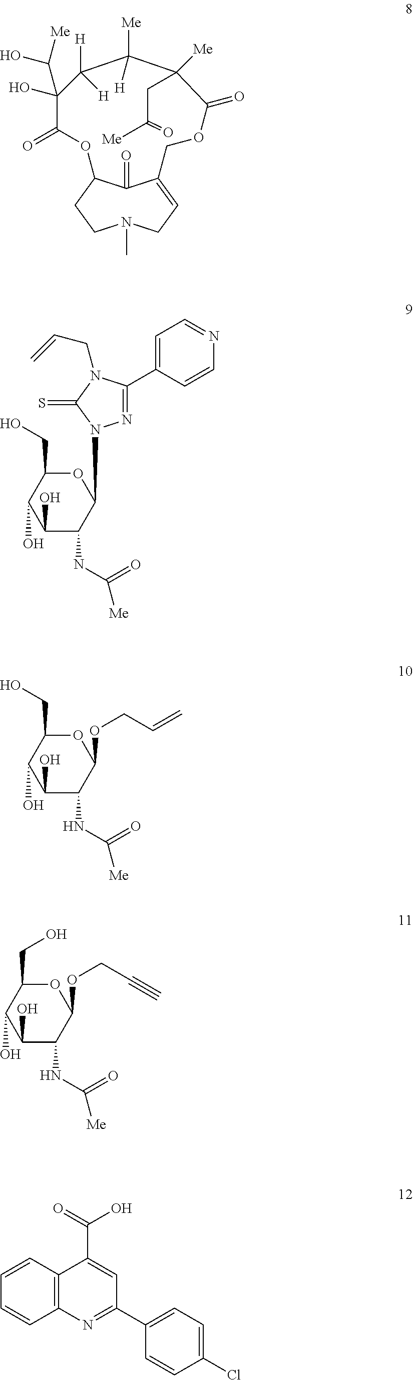

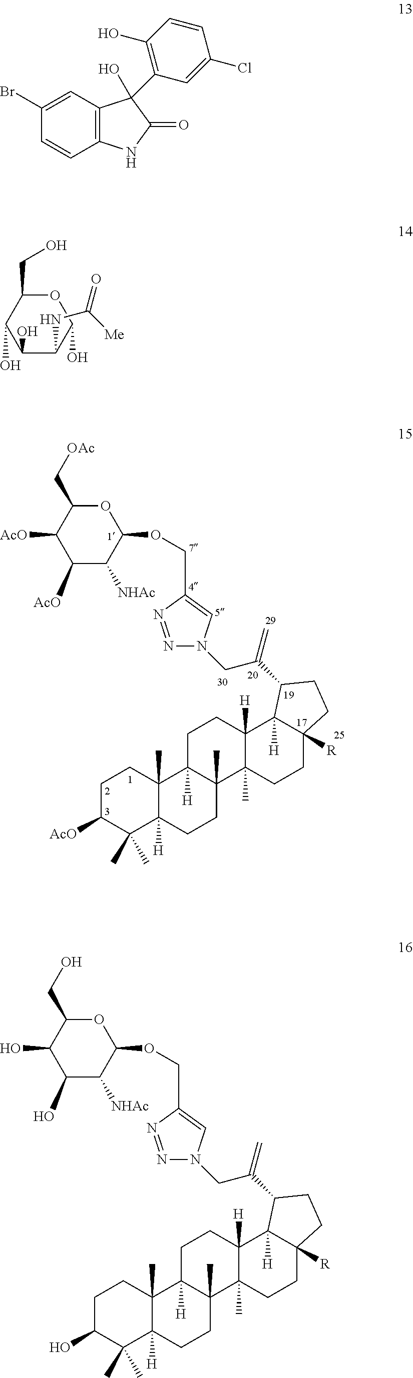

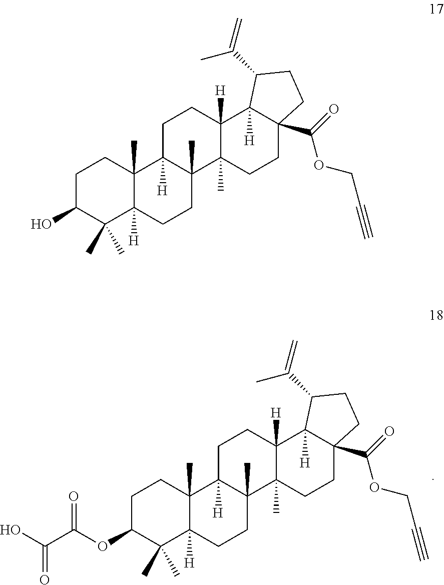

In various embodiments, in compounds 15 and 16, R is CH₂OAc, COOH, or CH₂OH. Compounds 2-18 can be conjugated/bonded to any suitable [CON], [Linker], or [Linker-2] as described herein and in Majouga, A. G., et al., “Identification of Novel Small-Molecule ASGP-R Ligands,” Current Drug Delivery, 2016, 13, 1303-1312, doi: 10.2174/1567201813666160719144651; Olshanova, A. S., et al., “Synthesis of a new betulinic acid glycoconjugate with N-acetyl-D-galactosamine for the targeted delivery to hepatocellular carcinoma cells,” Russian Chemical Bulletin, International Edition, Vol. 69, No. 1, pp. 158-163, January 2020; Yamansarov, E. Yu., et al., “New ASGPR-targeted ligands based on glycoconjugated natural triterpenoids,” Russian Chemical Bulletin, International Edition, Vol. 68, No. 12, pp. 2331-2338, December 2019. Compounds 2-18 can be attached through any suitable reactive group contained therein. Without limitation, compounds 2-13 can be attached to a CON], [Linker], or [Linker-2] through or by reaction with at least one OH, NH, vinyl, alkynyl, amide, acid, ester, ketone, or aromatic halogen contained in compounds 2-18. Suitable reaction modes for attaching compounds 2-18 to a [CON], [Linker], or [Linker-2] as described herein include, but are not limited to, substitution (e.g. alkylation of OH or NH groups), esterification (forming an ester), amidation (forming an amide), transesterification (exchanging one ester for another), transamidation (exchanging one amide for another), azide-alkyne cycloaddition, and other reactions capable of forming C—C, N—C, or O—C bonds with vinyl and alkynyl groups such as cycloadditions, aminations, oxidations, alkylations, rearrangement reactions (e.g. Claisen, Cope, etc.), and the like.

The disclosure encompasses polyclonal, monoclonal, synthetic antibodies, and the like. One skilled in the art would understand, based upon the disclosure provided herein, that an important feature of the antibody useful within the disclosure is that the antibody specifically bind with a circulating protein.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³O, ¹⁴O, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

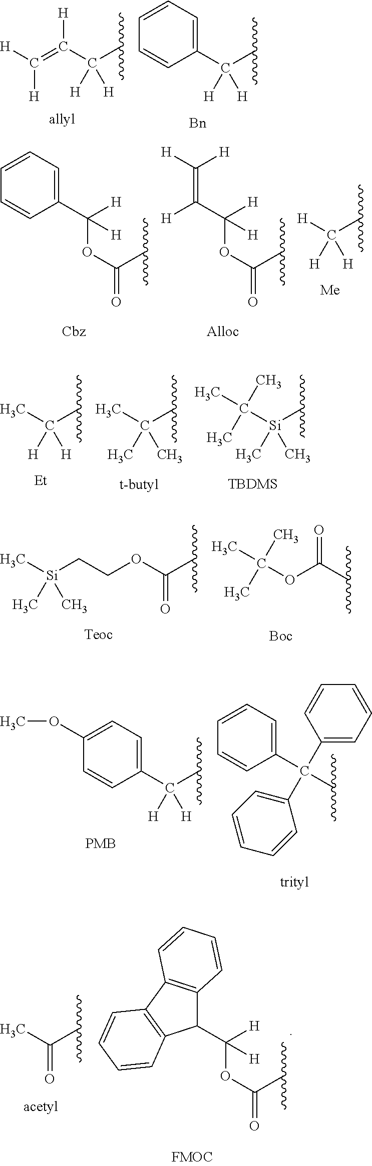

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

The compounds of the disclosure can be used to treat certain diseases and/or disorder, such as, but not limited to, autoimmune diseases, cancer, inflammation, or any other disease and/or disorder described herein.

Non-limiting examples of diseases and/or disorders include acute sciatic pain, advanced solid tumors, allergic asthma, allergic reaction, ALS and multiple sclerosis, Alzheimer's disease, amyloidosis, Anaplastic large-cell lymphoma, angioedema, angiogenesis, angiogenesis, ocular vascular diseases, ankylosing spondylitis, psoriasis, anthrax (prophylaxis and treatment), arthritis, asthma, asthma and white blood cell diseases, asthma, atopic dermatitis, atypical hemolytic uremic syndrome, autoimmune diseases, autoimmune disorders, B-cell cancers, B-cell malignancies, Bacillus anthracis spores, bleeding, bleeding with hemophilia, cancer, cancer (diagnosis), cancer etc., cancer, viral infections, chronic myelomonocytic leukemia and juvenile myelomonocytic leukemia, clear cell renal cell carcinoma, clinical signs of atopic dermatitis in dogs, cold agglutinin disease, colorectal cancer, Crohn's disease, psoriasis, psoriatic arthritis, and asthma, Rheumatoid arthritis, axial spondyloarthritis, psoriasis arthritis, ulcerative colitis, Cryopyrin-associated periodic syndrome, diabetes, diagnostic agent, diarrhoea caused by E. coli, Duchenne muscular dystrophy, dyslipidemia, eczema, fibrosis, gastric cancer or gastroesophageal junction adenocarcinoma, gastrointestinal cancers, geographic atrophy secondary to age-related macular degeneration, haemophilia A, hemophagocytic lymphohistiocytosis, high-risk neuroblastoma and refractory osteomedullary disease, Hodgkin's lymphoma, hypercholesterolemia, idiopathic pulmonary fibrosis, focal segmental glomerulosclerosis, cancer, immunologically mediated inflammatory disorders, infectious disease/influenza A, inflammation, inflammations of the airways, skin and gastrointestinal tract, inflammatory autoimmune diseases, inflammatory lesions and metastases (detection), influenza A, invasive Candida infection, macular degeneration (wet form), metastatic cancer, retinopathy of prematurity, metastatic pancreatic cancer, migraine, migraine and cluster headache, multiple sclerosis, multiple sclerosis, psoriasis, psoriatic arthritis, muscle atrophy due to orthopedic disuse and sarcopenia, muscle wasting disorders, muscular dystrophy, nosocomial pneumonia, oncology/immune indications, osteoarthritis in dogs, osteoarthritis, rheumatoid arthritis, osteomyelitis, osteoporosis, osteoporosis, bone metastases etc., ovarian cancer, ovarian cancer, pain, pain and inflammatory diseases, pancreatic and colorectal cancer, pancreatic cancer, Parkinson's disease, paroxysmal nocturnal hemoglobinuria, atypical hemolytic uremic syndrome, paroxysmal nocturnal hemoglobinuria, atypical HUS, Plaque psoriasis, primary systemic amyloidosis, progressive supranuclear palsy, psoriasis, inflammatory bowel diseases, multiple sclerosis, reduction of scarring after glaucoma surgery, reduction of side effects of cardiac surgery, relapsed or refractory cervical cancer, reversal of anticoagulant effects of dabigatran, rheumatoid arthritis, ankylosing spondylitis, rheumatoid arthritis, ankylosing spondylitis, psoriatic arthritis, psoriasis, Crohn's disease, ulcerative colitis, Rheumatoid arthritis, Crohn's disease, Plaque Psoriasis, Psoriatic Arthritis, Ankylosing Spondylitis, Juvenile Idiopathic Arthritis, Hemolytic disease of the newborn, psoriasis, rheumatoid arthritis, psoriatic arthritis, ankylosing spondylitis, sepsis, sepsis caused by Gram-negative bacteria, severe asthma and chronic spontaneous urticaria, SLE, dermatomyositis, polymyositis, solid malignancies, solid tumors, spinal cord injury and multiple sclerosis, Staphylococcus aureus infection, systemic lupus erythematosus, systemic lupus erythematosus without renal or CNS involvement, systemic lupus erythematosus, lupus nephritis, acute graft-versus-host disease, systemic scleroderma, thromboembolism, thrombotic thrombocytopenic purpura, thrombosis, triple-negative breast cancer, uveitis, rheumatoid arthritis psoriasis, viral infections, wet age-related macular degeneration, X-linked hypophosphatemia.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats the disease or disorder.

In certain embodiments, administering the compound(s) described herein to the subject allows for administering a lower dose of the additional therapeutic agent as compared to the dose of the additional therapeutic agent alone that is required to achieve similar results in treating the disease or disorder in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating the disease or disorder, and/or with an additional therapeutic agents that reduce or ameliorate the symptoms and/or side-effects of therapeutic agent used in the treatment of the disease or disorder. These additional therapeutic agents may comprise compounds that are commercially available or synthetically accessible to those skilled in the art. When the additional therapeutic agents useful for treating the disease or disorder are used, these additional therapeutic agents are known to treat, or reduce the symptoms of the disease or disorder.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, point out specific embodiments of the present disclosure, and are not to be construed as limiting in any way the remainder of the disclosure.

The materials and methods used in the experiments presented in this Experimental Example are now described in the present application.

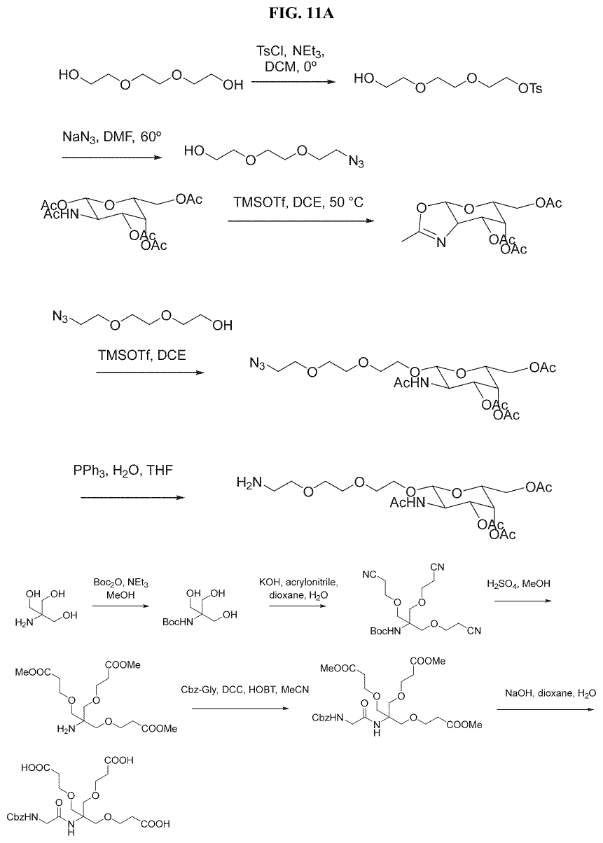

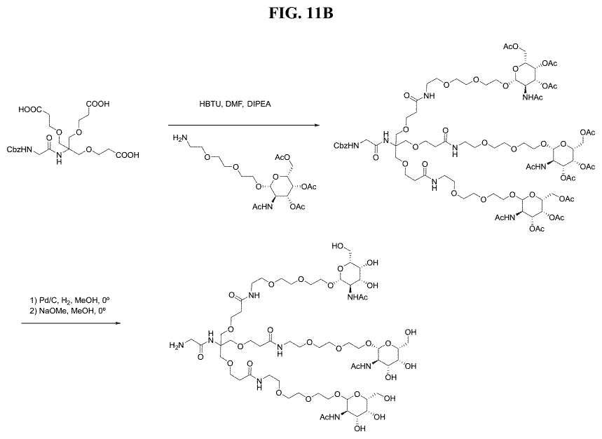

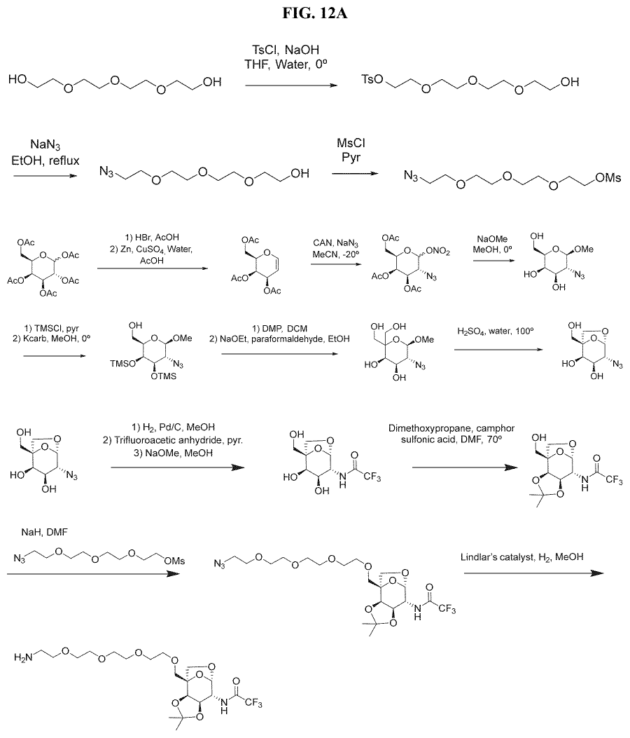

FIGS. 11A-11B illustrate the non-limiting synthesis of an ASGPRBM group.

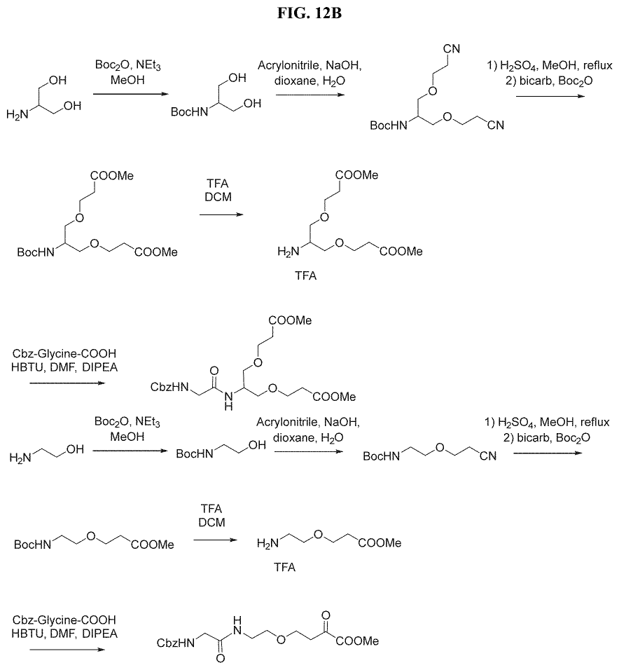

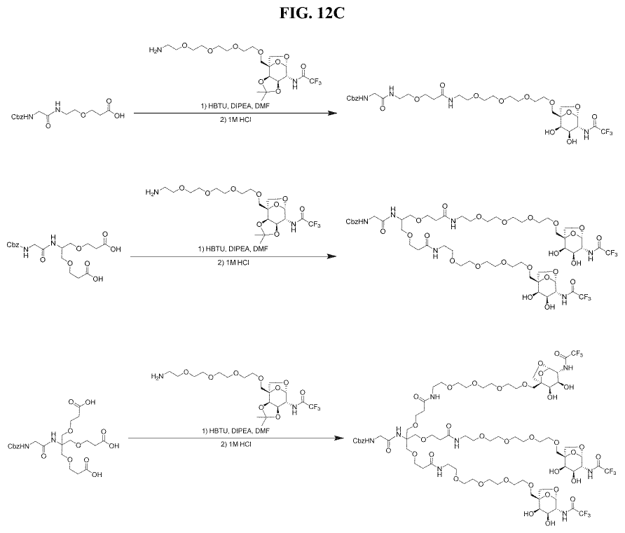

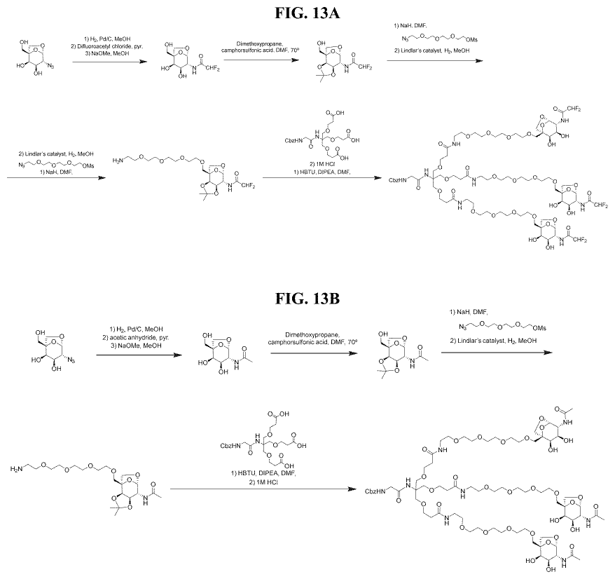

FIGS. 12A-12C illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

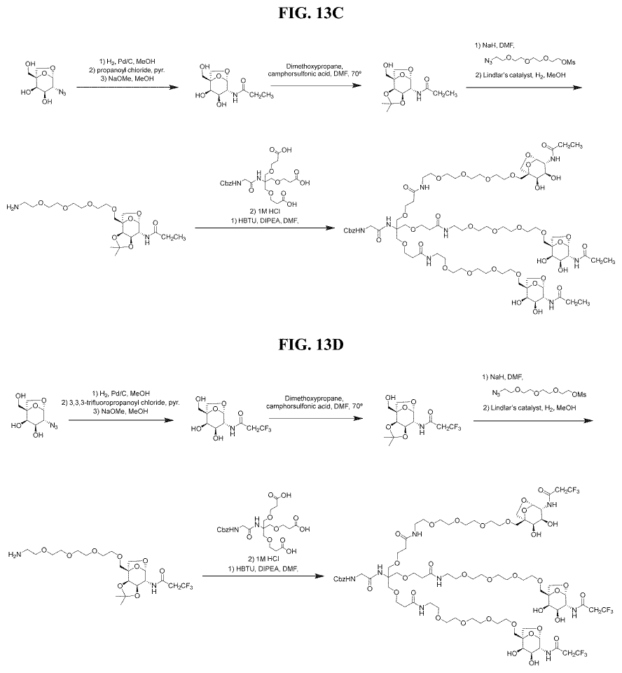

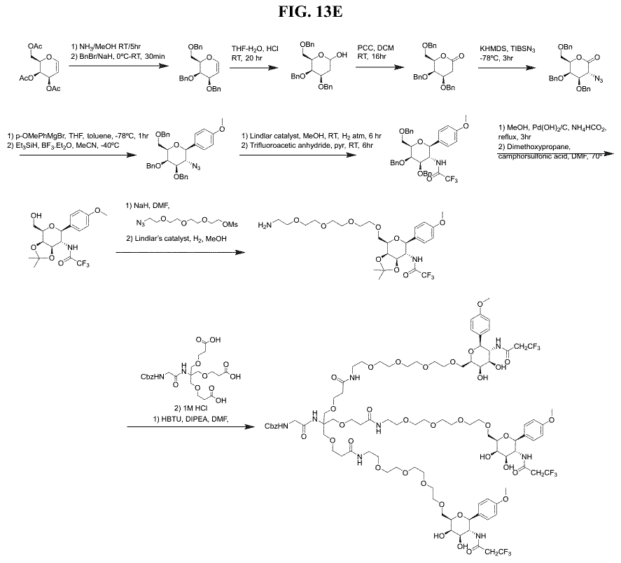

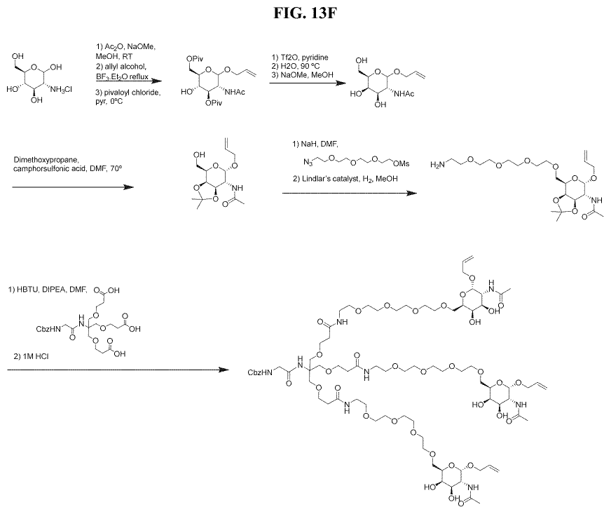

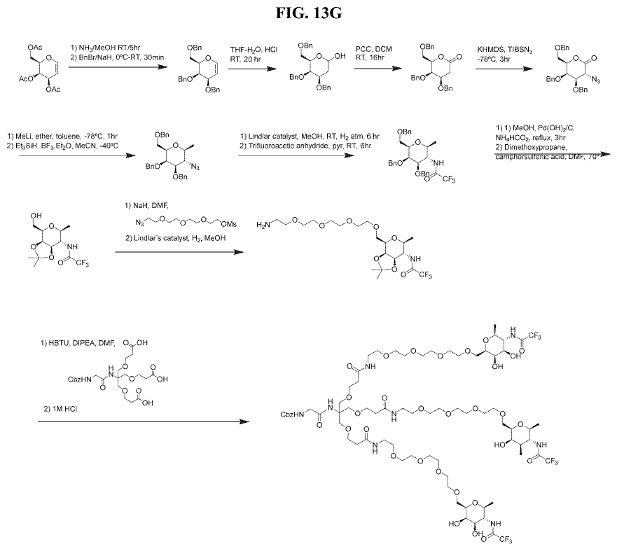

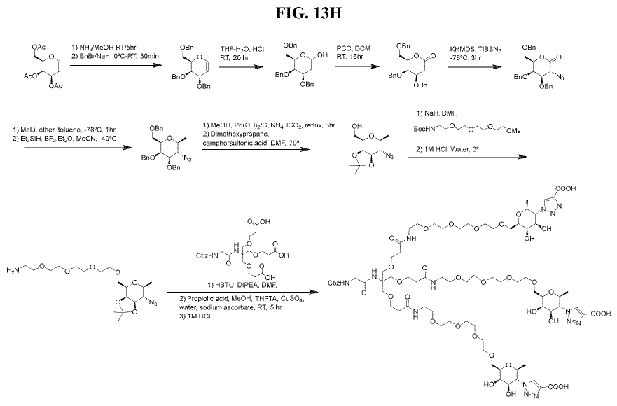

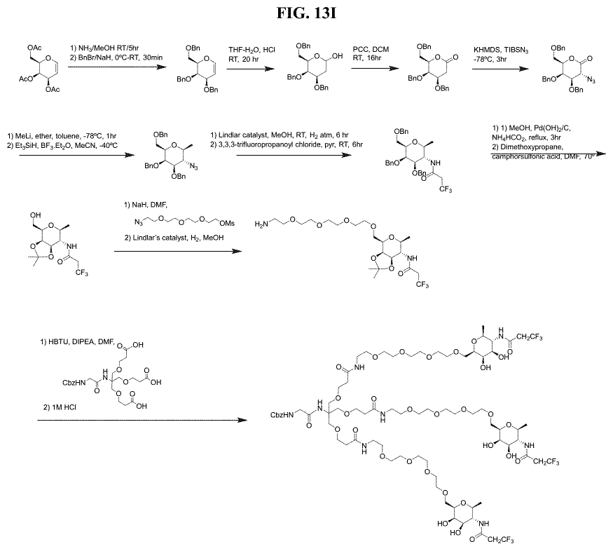

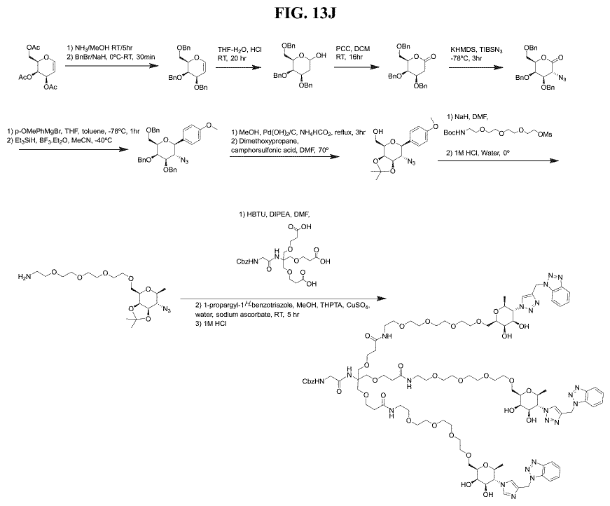

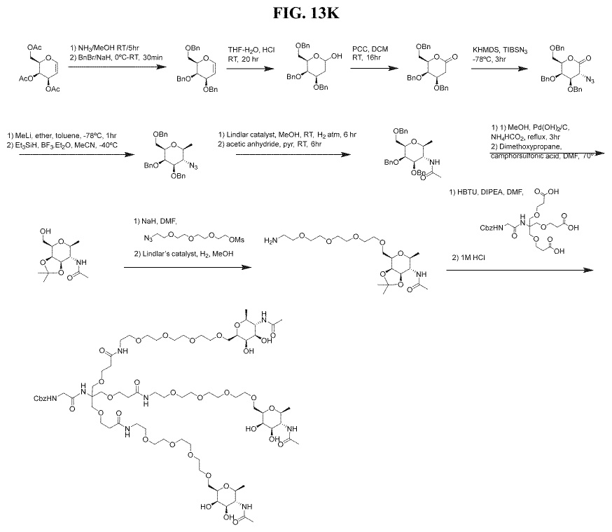

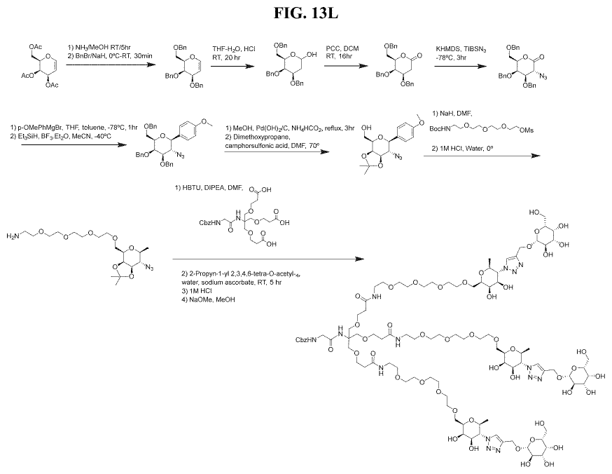

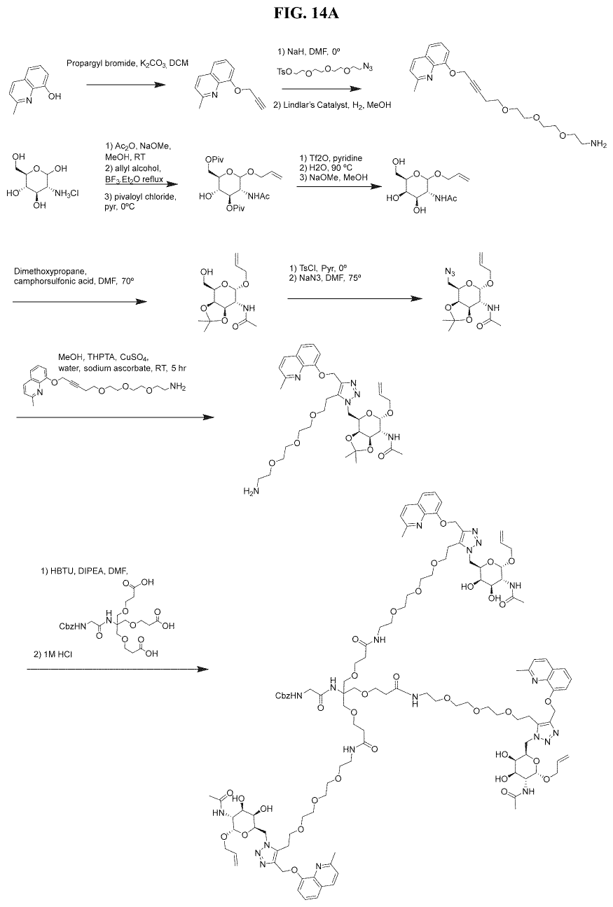

FIGS. 13A-13L illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

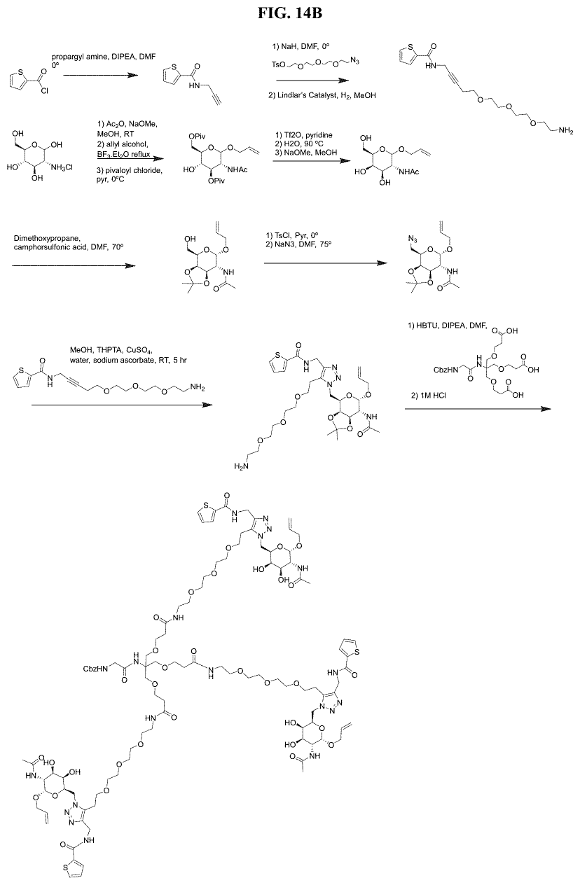

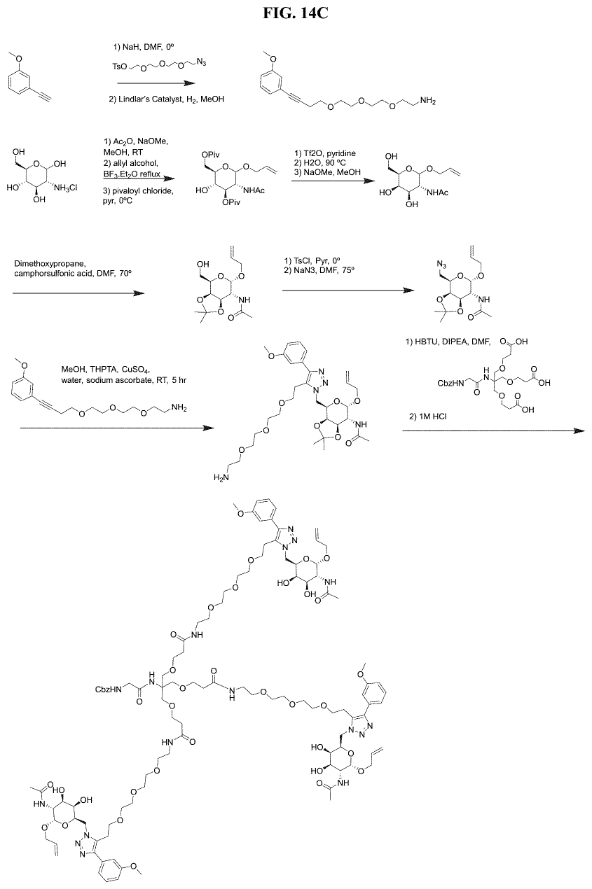

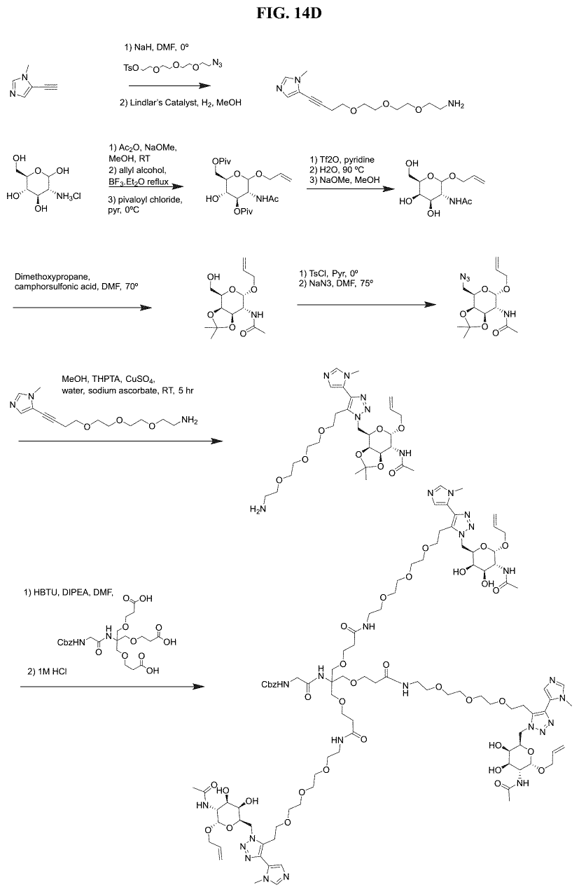

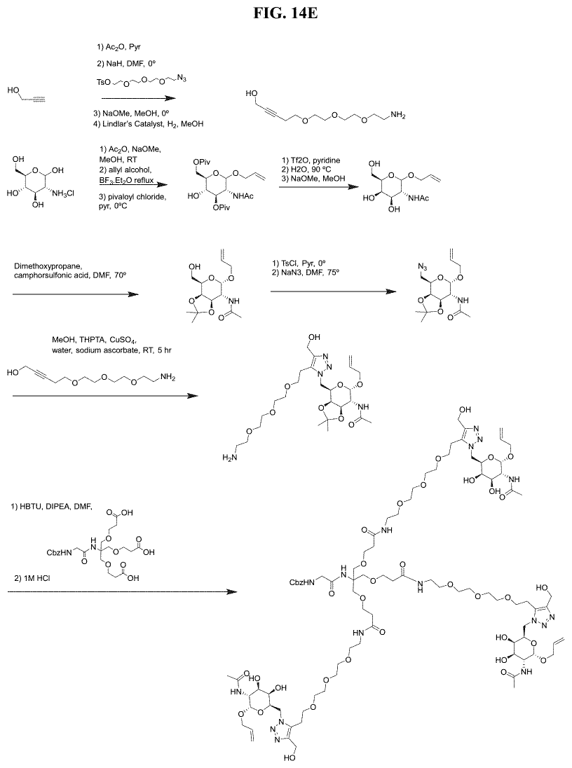

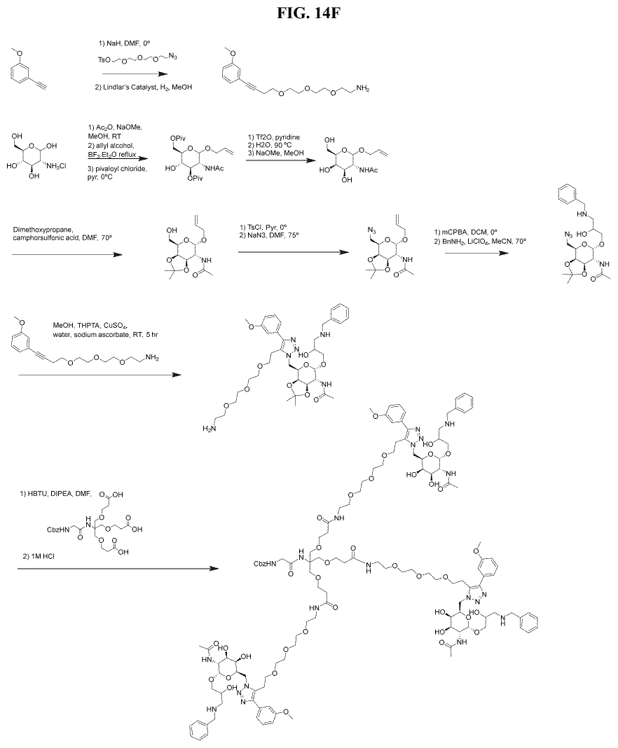

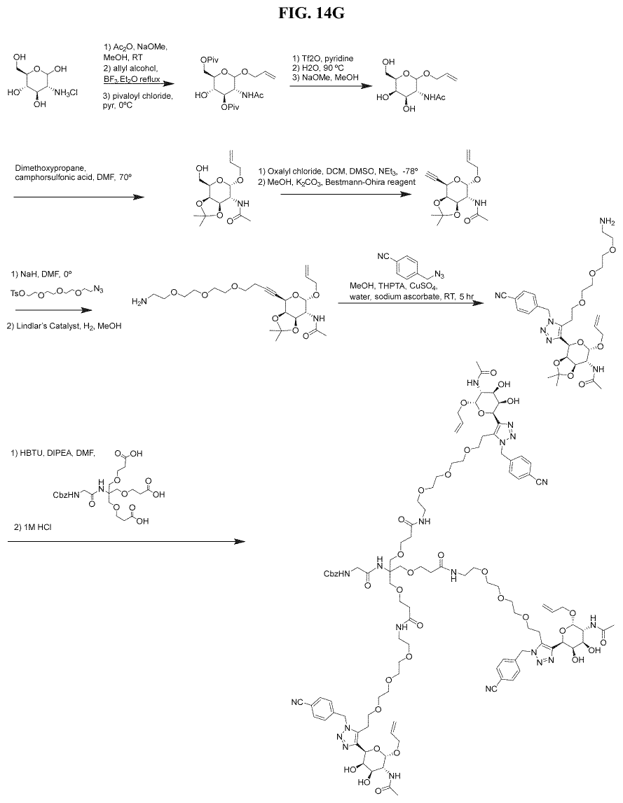

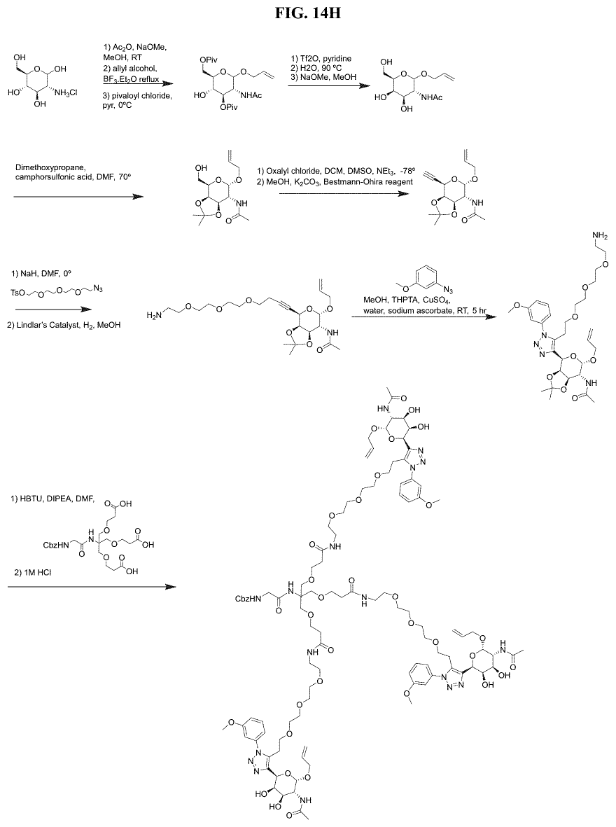

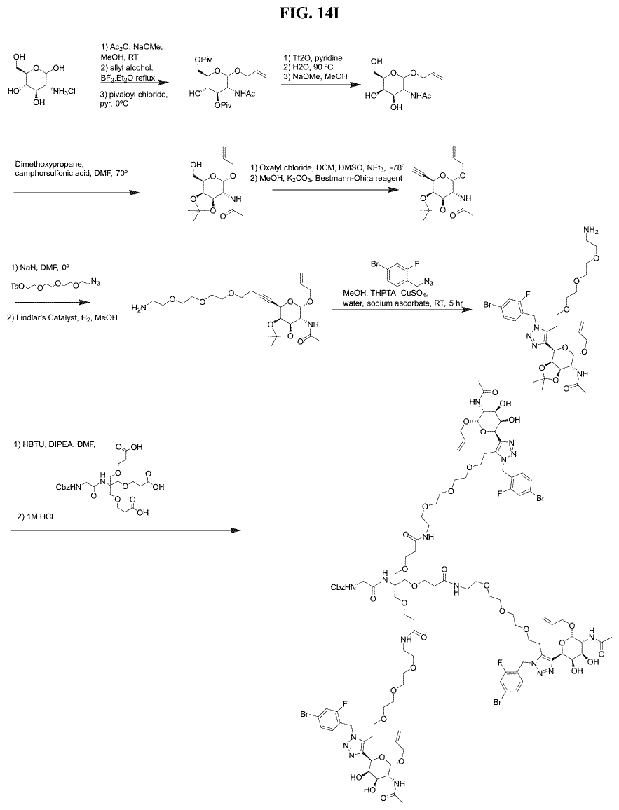

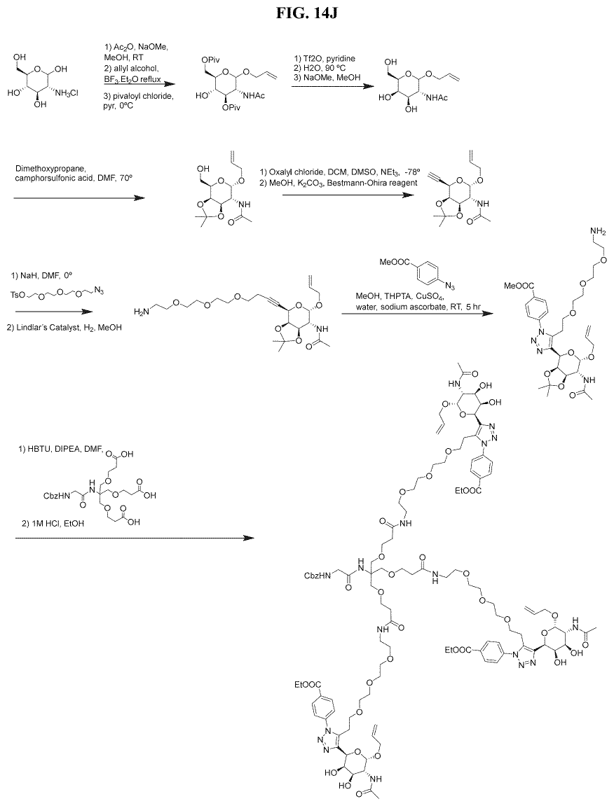

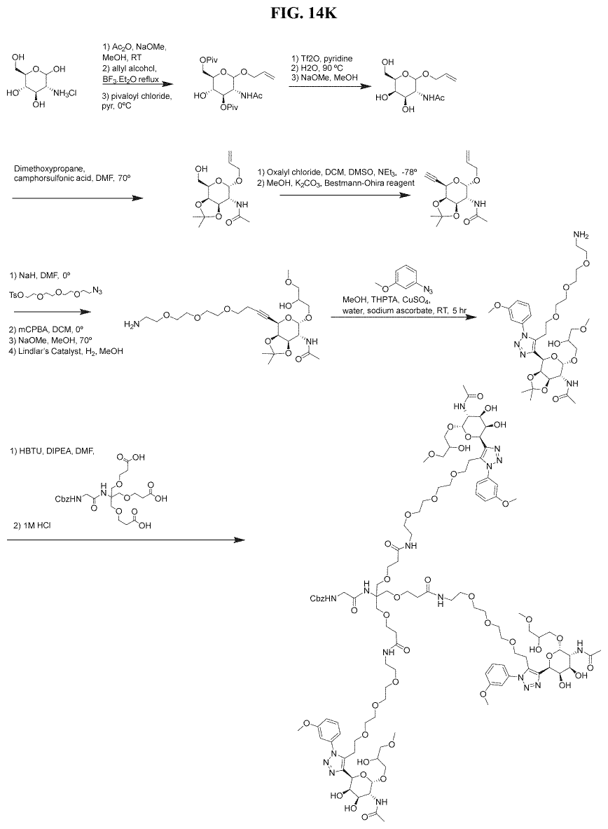

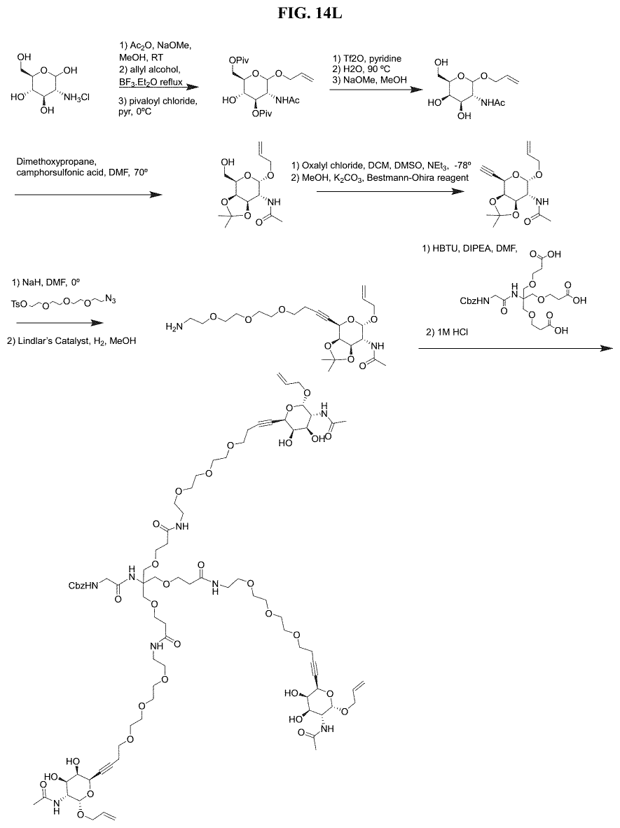

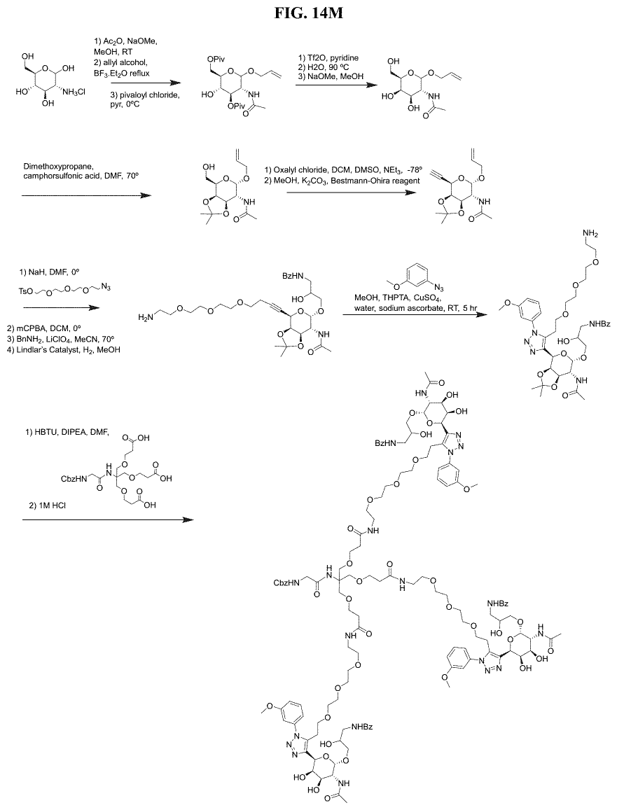

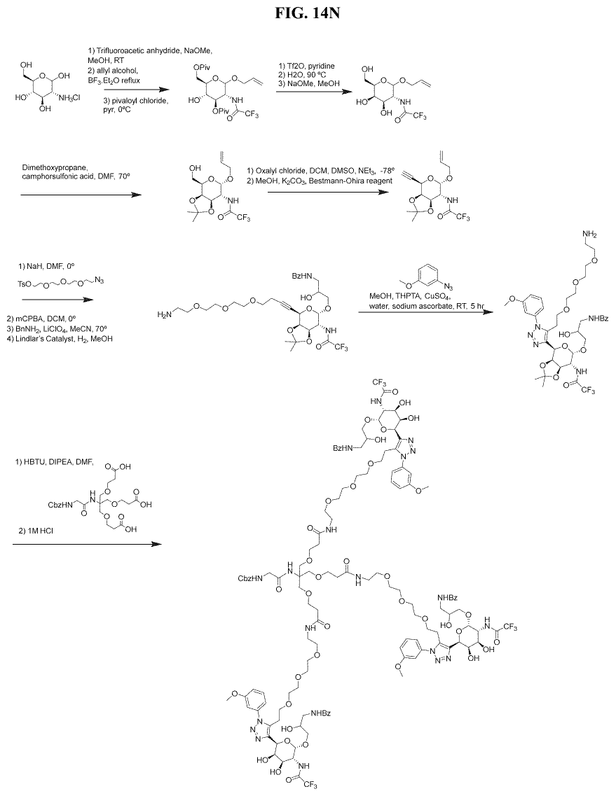

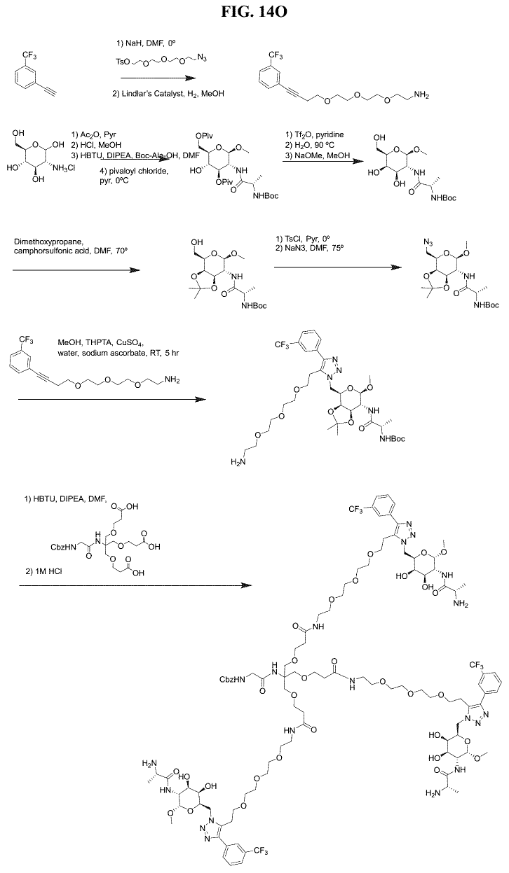

FIGS. 14A-14O illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

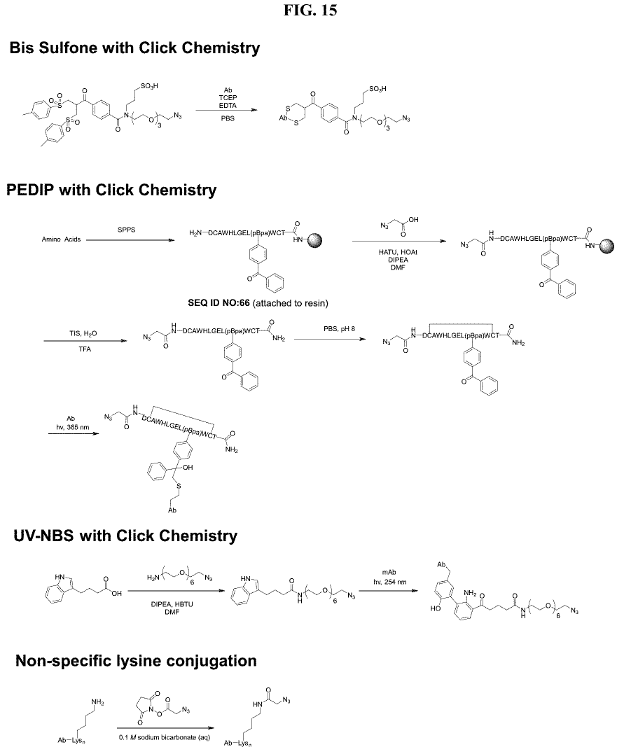

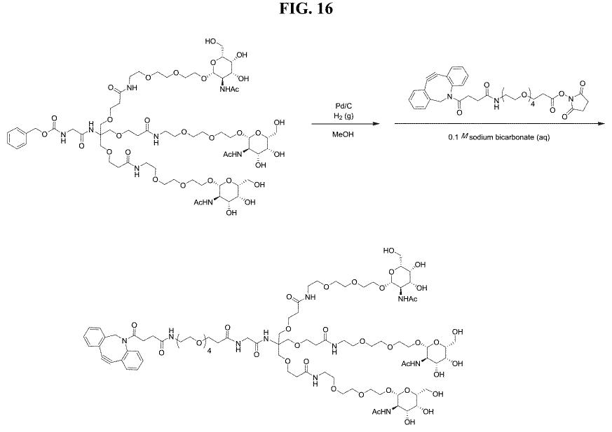

FIG. 15 illustrates non-limiting synthetic schemes that allow for labeling (derivatization) of an antibody (labeled as Ab) with an azido group, wherein pBpa represents para-benzoyl-phenylalanine.

FIG. 16 illustrates a non-limiting synthetic scheme that allow for labeling (derivatization) of a CRBM group with a strained alkyne containing group. In certain non-limiting embodiments, any azido-containing compound (such as, but not limited to, those shown in FIG. 15) can react with a strained alkyne-containing compound (such as, but not limited to, that shown in FIG. 16), so as to generate a compound of the disclosure.

### Enumerated Embodiments:

The following enumerated embodiments are provided, the numbering of which is not to be construed as designating levels of importance.

Embodiment 1 provides a compound comprising formula (I), or a salt, geometric isomer, stereoisomer, or solvate thereof:

[Ab]_{k′}—[CON]ₕ-[Linker]ᵢ-[CON]_{h′}—[CRBM]_{j′} (I),

wherein:

the Ab is an antibody that binds to an extracellular protein;

the CRBM is a cellular receptor binding moiety that binds to at least one receptor on the surface of a degrading cell in a subject, whereby binding of (I) leads to endocytosis and degradation of the extracellular protein;

each CON is independently a bond or a group that covalently links an Ab to an CRBM, an Ab to a Linker, and/or a Linker to a CRBM;

the Linker is a group having a valence ranging from 1 to 15;

k′ is an integer ranging from 1 to 15;

h is an integer ranging from 0 to 15;

i is an integer ranging from 0 to 15;

h′ is an integer ranging from 0 to 15;

j is an integer ranging from 1 to 15.

Embodiment 2 provides the compound of Embodiment 1, wherein the valence of the Linker is 1, 2, or 3.

Embodiment 3 provides the compound of any one of Embodiments 1-2, wherein k′ is 1, 2, or 3.

Embodiment 4 provides the compound of any one of Embodiments 1-3, wherein j is 1, 2, or 3.

Embodiment 5 provides the compound of any one of Embodiments 1-4, wherein h is 1, 2, or 3.

Embodiment 6 provides the compound of any one of Embodiments 1-5, wherein h′ is 1, 2, or 3.

Embodiment 7 provides the compound of any one of Embodiments 1-6, wherein i is 1, 2, or 3.

Embodiment 8 provides the compound of any one of Embodiments 1-7, wherein at least one of h, h′, and i is at least 1.

Embodiment 9 provides the compound of any one of Embodiments 1-8, wherein k′, j′, h, h′, and i are each independently 1, 2, or 3.

Embodiment 10 provides the compound of any one of Embodiments 1-9, wherein k′ is 1, and j′ is 1, 2, or 3.

Embodiment 11 provides the compound of any one of Embodiments 1-10, which is:

[Ab]-[CON]₀₋₁-[Linker]-[CON]₀₋₁—[CRBM] (Ia).

Embodiment 12 provides the compound of any one of Embodiments 1-11, wherein the degrading cell comprises a hepatocyte.

Embodiment 13 provides the compound of any one of Embodiments 1-12, wherein the CRBM is a folic acid (folate) receptor binder, mannose receptor binder, mannose-6-phosphate (M6P) receptor binder, low density lipoprotein receptor-related protein 1 (LRP1) receptor binder, low density lipoprotein receptor (LDLR) binder, FcγRI receptor binder, transferrin receptor binder, macrophage scavenger receptor binder, G-Protein coupled receptor binder, or asialoglycoprotein receptor (ASGPR) binder.

Embodiment 14 provides the compound of any one of Embodiments 1-13, wherein the CRBM is:
(a) a folic acid (folate) receptor binder comprising at least one of folic acid, methotrexate, premetrexed, or a biologically active fragment thereof;
(b) a mannose receptor binder comprising at least one of:

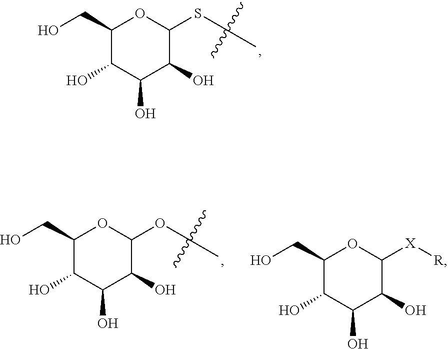

wherein: X is S or O, R is selected from the group consisting of:

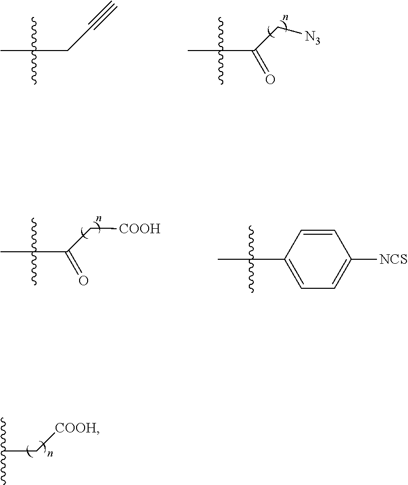

and each occurrence of ‘n’ is independently 1, 2, 3, 4, 5, 6, 7, 8, 9, 10, 11, 12, 13, 14, 15, 16, 17, 18, 19, or 20; and
a polymeric molecule illustrated in FIGS. 1-7;
(c) a mannose-6-phosphate (M6P) receptor binder comprising at least one of:

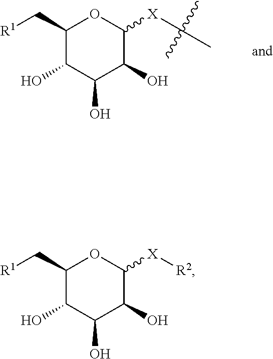

wherein X is O or S, R¹ is selected from the group consisting of:

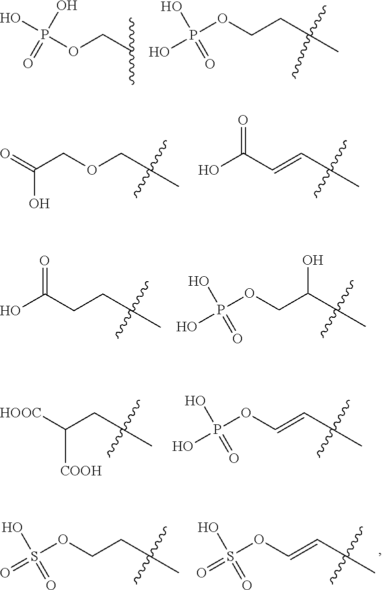

R² is selected from the group consisting of:

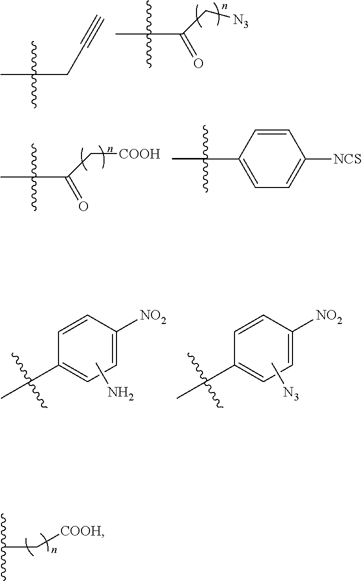

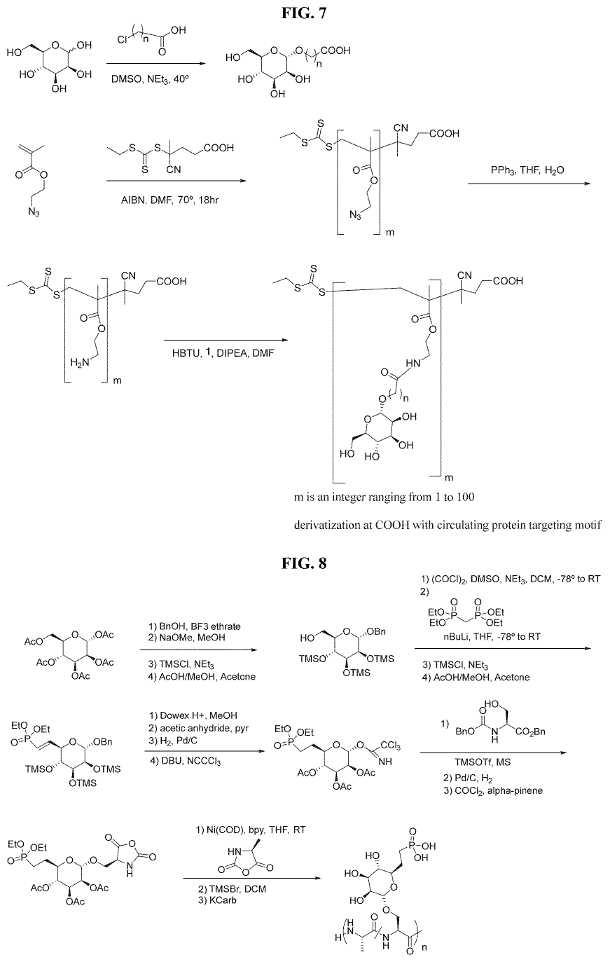

and each occurrence of ‘n’ is independently 1, 2, 3, 4, 5, 6, 7, 8, 9, 10, 11, 12, 13, 14, 15, 16, 17, 18, 19, or 20;
a polymeric molecule illustrated in FIG. 8;
a compound selected from:

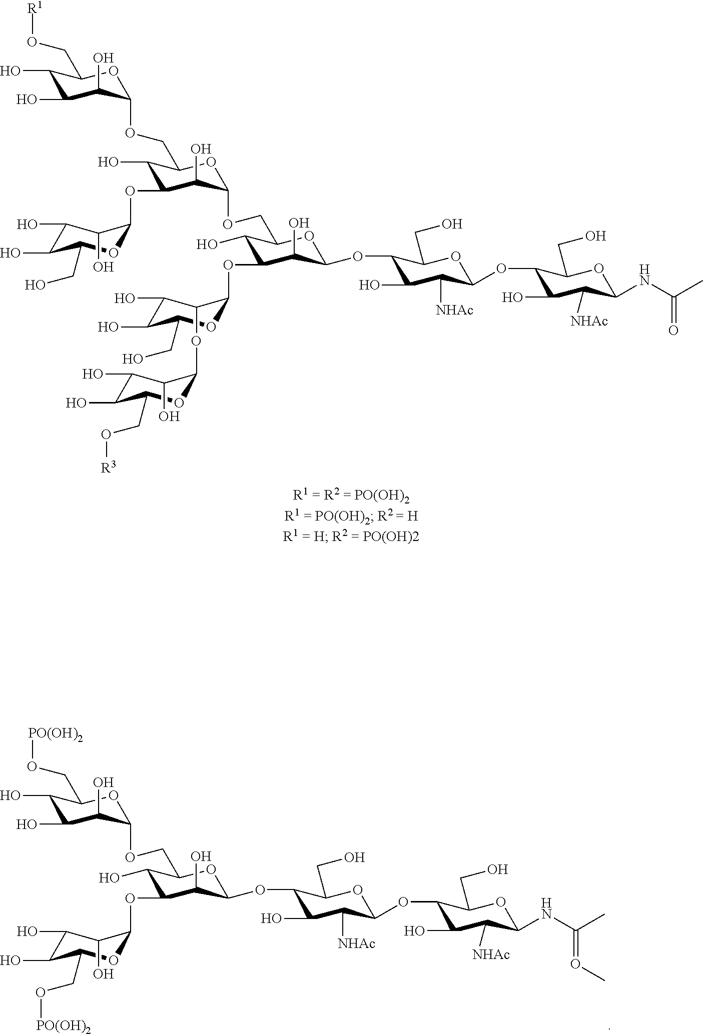

a compound selected from:

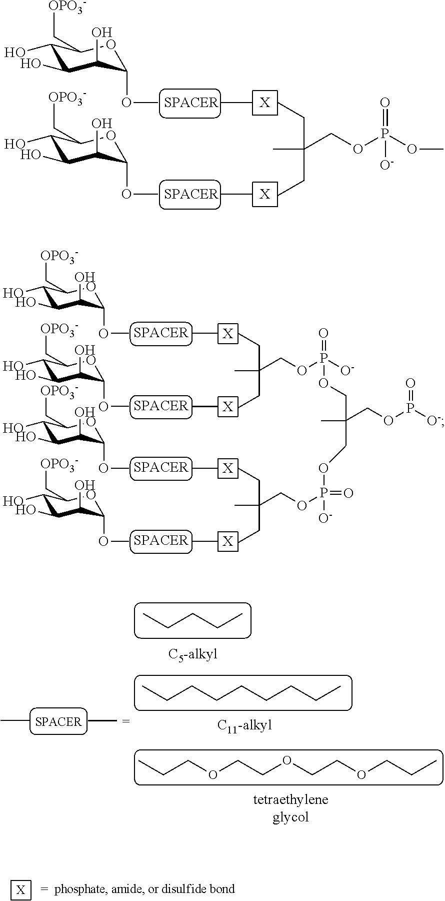

(d) a low density lipoprotein receptor-related protein 1 (LRP1) receptor binder comprising at least one amino acid sequence of SEQ ID NOs:1-9;
(e) a low density lipoprotein receptor (LDLR) binder comprising at least one amino acid sequence of SEQ ID NOs:10-35;
(f) a FcγRI receptor binder comprising at least one amino acid sequence of SEQ ID NOs:36-52;
(g) a transferrin receptor binder comprising at least one amino acid sequence of SEQ ID NOs:53-59 and 67;
(h) a macrophage scavenger receptor binder comprising at least one amino acid sequence of SEQ ID NOs:60-65;
(i) a G-protein coupled receptor binder comprising at least one of:

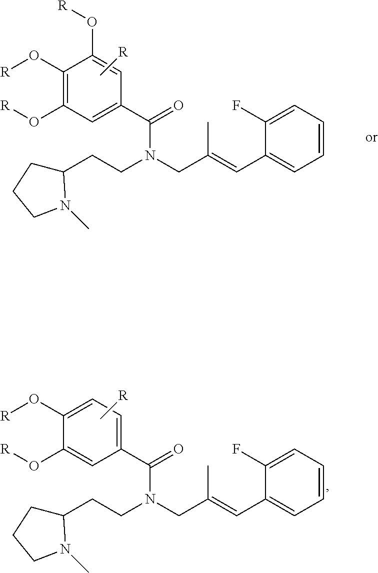

wherein each occurrence of R is independently H or C₁-C₆ alkyl;
(j) an asialoglycoprotein receptor (ASGPR) binder comprising:

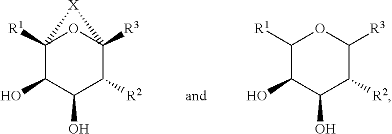

wherein:
X is a linker of 1-4 atoms in length and comprises O, S, N(R^{N1}), or C(R^{N1})(R^{N1}) groups, such that:
   when X is a linker of 1 atom in length, X is O, S, N(R^{N1}), or C(R^{N1})(R^{N1}),
   when X is a linker of 2 atoms in length, no more than 1 atom of X is O, S, or N(R^{N1}),
   when X is a linker of 3 or 4 atoms in length, no more than 2 atoms of X are independently O, S, or N(R^{N1});
   wherein each occurrence of R^{N1} is independently H or C₁-C₃ alkyl optionally substituted with 1-3 independently selected halogens and/or 1-2 hydroxyl groups;
R¹ and R³ are each independently H, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}(vinyl), —O(CH₂)_{K}(vinyl), —(CH₂)_{K}(alkynyl), —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens; or
R¹ and R³ are each independently Ph(CH₂)_{K}—, which is optionally substituted with: 1-3 independently selected halogens; C₁-C₄ alkyl optionally substituted with 1-3 independently selected halogens and/or 1-2 hydroxyl groups; or C₁-C₄ alkoxy optionally substituted with 1-3 independently selected halogens and/or 1-2 hydroxyl groups; or
R¹ and R³ are each independently a group of structure:

—O—(CH₂)_{K}—CH(OH)—(CH₂)K′—R⁷,
wherein:
   R⁷ is: C₁-C₄ alkoxy optionally substituted with 1-3 independently selected halogens and/or 1-2 hydroxy groups; —NR^{N3}R^{N4}; or —(CH₂)_{K}—O—(CH₂)_{K}—CH₂—CH═CH₂;
   K is 0, 1, 2, 3, or 4;
   K′ is 1, 2, 3, or 4;
   each occurrence of R^{N3} is independently H or C₁-C₃ alkyl optionally substituted with 1-3 independently selected halogens and/or 1-2 hydroxyl groups;
   each occurrence of R^{N4} is independently H, C₁-C₃ alkyl optionally substituted with 1-3 independently selected halogens and/or 1-2 hydroxyl groups, or Ph-(CH₂)_{K}—; or
R¹ and R³ are each independently selected from the group consisting of:
   —(CH₂)_{K}OH,

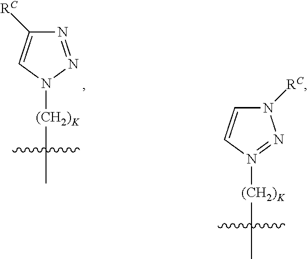

L¹—≡—, L¹—(CH₂)_{K}—, and CYC—(CH₂)_{K}—,
wherein CYC is selected from the group consisting of:

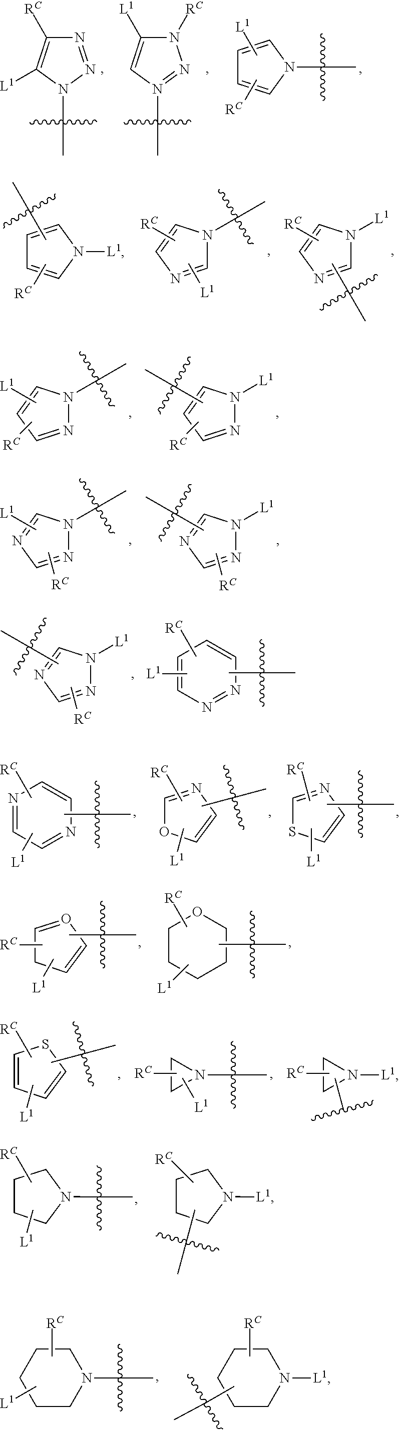

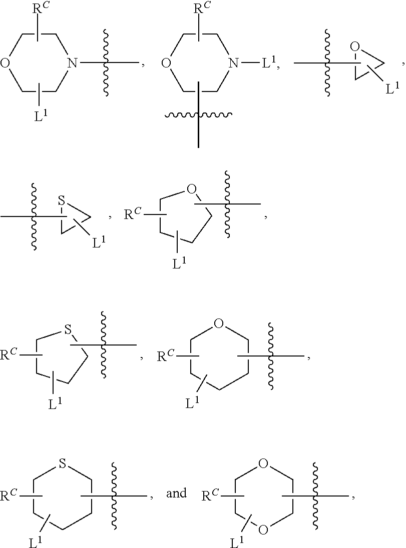

wherein:
   the bond marked with [symbol] indicating the site on CYC whereto —(CH₂)_{K} is connected;
   L¹ is a bond, -Linker, —CON-Linker, or —CON-Linker-CON;
   R^{C} is absent, H, C₁-C₄ alkyl optionally substituted with 1-3 optionally substituted halogens and/or 1-2 hydroxyl groups, or a group of structure:

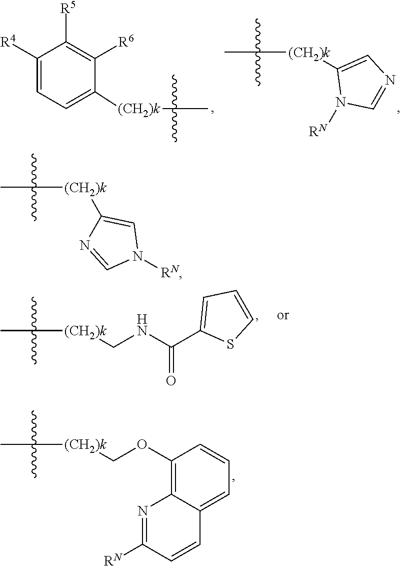

wherein:
   R⁴, R⁵, and R⁶ are each independently H, F, Cl, Br, I, CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens, C₁-C₃-alkoxy optionally substituted with 1-3 independently selected halogens, —(CH₂)_{K}COOH, —(CH₂)_{K}C(═O)O—(C₁-C₄ alkyl) optionally substituted with 1-3 independently selected halogens, O—C(═O)—(C₁-C₄ alkyl) optionally substituted with 1-3 independently selected halogens, or —C(═O)—(C₁-C₄ alkyl) optionally substituted with 1-3 independently selected halogens;
   each occurrence of R^{N} is independently H or C₁-C₃ alkyl optionally substituted with 1-3 independently selected halogens and/or 1-2 hydroxyl groups;
   each occurrence of R^{N2} is independently H or C₁-C₃ alkyl optionally substituted with 1-3 independently selected halogens and/or 1-2 hydroxyl groups;
   or
R¹ and R³ are each independently (C₃-C₈ saturated carbocyclic)—(CH₂)_{K}—, wherein the carbocyclic is further substituted with —L¹ and —R^{C};
R² is —(CH₂)_{K}—N(R^{N1})—C(═O)R^{AM}, wherein:
   R^{AM} is H, C₁-C₄ alkyl optionally substituted with 1-3 independently selected halogens and/or 1-2 hydroxyl groups, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, —C(═O)(C₁-C₄ alkyl) optionally substituted with 1-3 independently selected halogens, or —(CH₂)_{K}—NR^{N3}R^{N4}; or
R² is

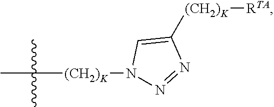

wherein:
R^{TA} is H, CN, NR^{N1}R^{N2}, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens, or
R^{TA} is C₃-C₁₀ aryl or a 3- to 10-membered heteroaryl group containing 1-5 non-carbon ring atoms, each of the aryl or heteroaryl groups being optionally substituted with 1-3 groups independently selected from CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens and/or 1-2 hydroxyl groups, —(C₁-C₃-alkoxy) optionally substituted from 1-3 independently selected halogens, —(CH₂)_{K}COOH, —(CH₂)_{K}C(═O)O—(C₁-C₄ alkyl) optionally substituted with 1-3 independently selected halogens, —OC(═O)(C₁-C₄ alkyl) optionally substituted with 1-3 independently selected halogens, or —(CH₂)_{K}C(═O)—(C₁-C₄ alkyl) optionally substituted with 1-3 independently selected halogens, or
R^{TA} is

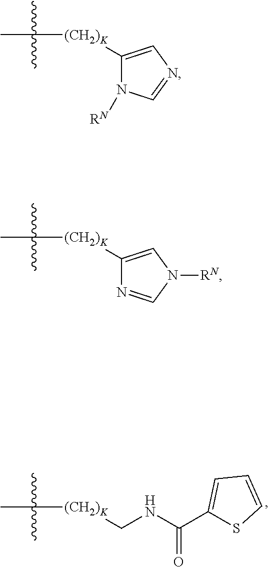

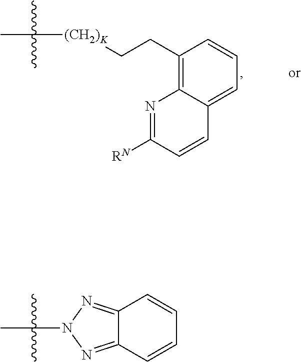

optionally substituted with 1-3 C₁-C₃ alkyl groups optionally substituted with 1-3 independently selected halogens, or
R^{TA} is

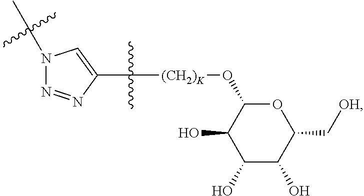

wherein each —(CH₂)_{K} group is optionally substituted with 1-4 C₁-C₃ alkyl groups optionally substituted with 1-3 fluoro groups or 1-2 hydroxyl groups.

Embodiment 15 provides the compound of Embodiments 1-14, wherein:
the X in ASGPRBM is —O—C(R^{N1})(R^{N1})—, —C(R^{N1})(R^{N1})—O—, —S—C(R^{N1})(R^{N1})—, —C(R^{N1})(R^{N1})—S—, —N(R^{N1})—C(R^{N1})(R^{N1})—, —C(R^{N1})(R^{N1})—N(R^{N1})—, or —C(R^{N1})(R^{N1})—C(R^{N1})(R^{N1})— when X is 2 atoms in length;
the X in ASGPRBM is —O—C(R^{N1})(R^{N1})—C(R^{N1})(R^{N1})—, —C(R^{N1})(R^{N1})—O—C(R^{N1})(R^{N1})—, —O—C(R^{N1})(R^{N1})—O—, —O—C(R^{N1})(R^{N1})—S—, —O—C(R^{N1})(R^{N1})—N(R^{N1})—, —S—C(R^{N1})(R^{N1})—C(R^{N1})(R^{N1})—, —C(R^{N1})(R^{N1})—S—C(R^{N1})(R^{N1})—, —C(R^{N1})(R^{N1})—C(R^{N1})(R^{N1})—S, —S—C(R^{N1})(R^{N1})—S—, —S—C(R^{N1})(R^{N1})—O—, —S—C(R^{N1})(R^{N1})—N(R^{N1})—, —N(R^{N1})—C(R^{N1})(R^{N1})—C(R^{N1})(R^{N1})—, —C(R^{N1})(R^{N1})—N(R^{N1})—C(R^{N1})(R^{N1})—, —C(R^{N1})(R^{N1})—C(R^{N1})(R^{N1})—N(R^{N1})—, —N(R^{N1})—C(R^{N1})(R^{N1})—N(R^{N1})—, or —C(R^{N1})(R^{N1})—C(R^{N1})(R^{N1})—C(R^{N1})(R^{N1}), when X is 3 atoms in length; or
the X in ASGPRBM is —O—C(R^{N1})(R^{N1})—C(R^{N1})(R^{N1})—C(R^{N1})(R^{N1})—, —C(R^{N1})(R^{N1})—O—C(R^{N1})(R^{N1})—C(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})—, —C(R^{N1})(R^{N1})—S—C(R^{N1})(R^{N1})—C(R^{N1})(R^{N1})—, —C(R^{N1})(R^{N1})—C(R^{N1})(R^{N1})—S—C(R^{N1})(R^{N1})—, —S—C(R^{N1})(R^{N1})—S—C(R^{N1})(R^{N1})—, —N(R^{N1})—C(R^{N1})(R^{N1})—C(R^{N1})(R^{N1})—C(R^{N1})(R^{N1})—, or —C(R^{N1})(R^{N1})—N(R^{N1})—C(R^{N1})(R^{N1})—C(R^{N1})(R^{N1})—, when X is 4 atoms in length.

Embodiment 16 provides the compound of any one of Embodiments 1-15, wherein X is OCH₂ and R^{N1} is H, or wherein X is CH₂O and R^{N1} is H.

Embodiment 17 provides the compound of any one of Embodiments 14-16, wherein the ASGPRBM comprises the structure:

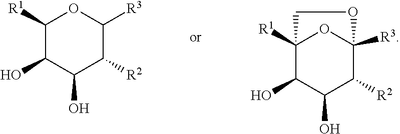

Embodiment 18 provides the compound of any one of Embodiments 14-17, wherein the ASGPRBM group comprises:

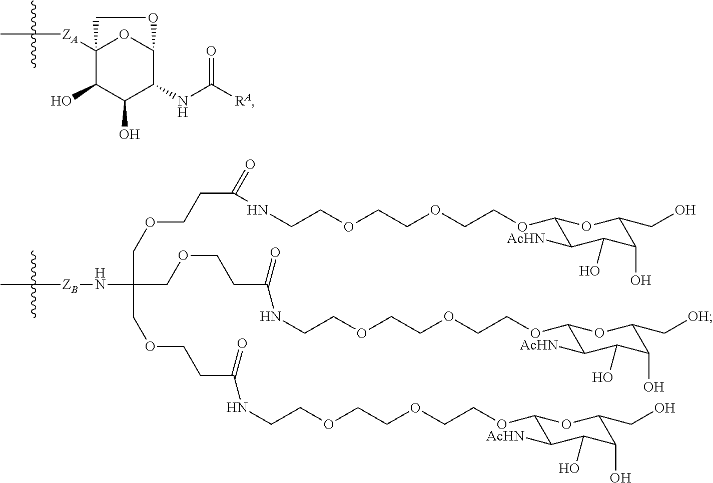

wherein:
R^{A} is C₁-C₃ alkyl optionally substituted with 1-5 independently selected halogens;
Z_{A} is —(CH₂)_{IM}—, —O—(CH₂)_{IM}—, —S—(CH₂)_{IM}—, —NR^{M}—(CH₂)_{IM}—, —C(═O)—(CH₂)_{IM}—, a PEG group containing from 1 to 8 ethylene glycol residues, or —C(O)(CH₂)_{IM}NR^{M}—;
Z_{B} is absent, —(CH₂)_{IM}—, —C(═O)—(CH₂)_{IM}—, or —C(═O)(CH₂)_{IM}—NR^{M}—;
R^{M} is H or C₁-C₃ alkyl optionally substituted with 1-2 hydroxyl groups; and each occurrence of IM is independently 0, 1, 2, 3, 4, 5, or 6;

3

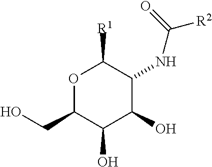

4

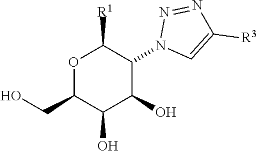

| | R¹ | # | R² | # | R² | # | R³ | # | R³ | # | R³ |
|---|---|---|---|---|---|---|---|---|---|---|---|
| a | Me | 1 | Me | 9 | CH₂NH₂ | 1 | Ph | 10 | 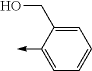 | 14 | 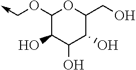 |
| b | 4- OMePh | 2 | [symbol] | 10 | CH₂CF₃ | 2 | 4-OMePh | 11 | 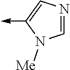 | 15 | CH₂OH |
| | | 3 | i-Pr | 11 | 2-furyl | 3 | 3-OMePh | 12 | 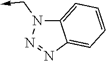 | 16 | CH₂NH₂ |
| | | 4 | t-Bu | 12 | Ph | 4 | 3-NH₂Ph | 13 | 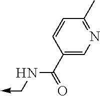 | 17 | CH₂NHMe |
| | | 5 | CCl₂ | 13 | 4-OMePh | 5 | 4-NMe₂Ph | | | 18 | CH₂NMe₂ |
| | | 6 | CF₃ | 14 | 3-OMePh | 6 | 2-pyridyl | | | 19 | CO₂H |
| | | 7 | Ot-Bu | 15 | 4-CNPh | 7 | 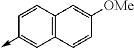 | | | 20 | CH₂NHCOPh |
| | | 8 | CH₂CO₂H | 16 | 3-pyridyl | 8 | 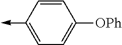 | | | 21 | CH₂NHCOMe |
| | | | | 17 | 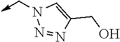 | 9 | 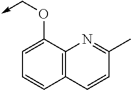 | | | | |

| | | R¹ | | R¹ |
|---|---|---|---|---|
| 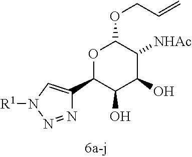 | a | Me | g | 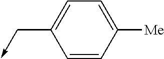 |
| | b | 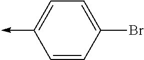 | h | 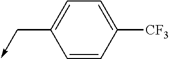 |
| | c | 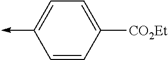 | i | 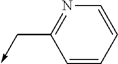 |
| | d | 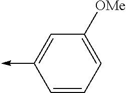 | j | 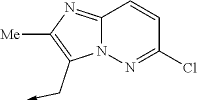 |
| | e | 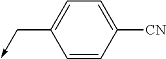 | | |
| | f | 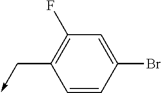 | | |
| | | R² | | R² |
| 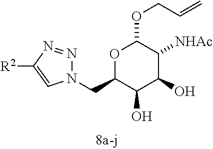 | a | CH₂OH | g | 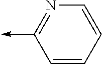 |
| | b | 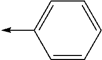 | h | 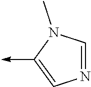 |
| | c | 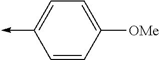 | i | 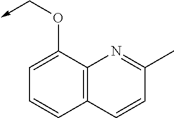 |
| | d | 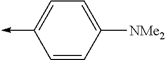 | j | 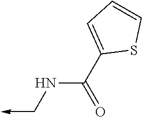 |
| | e | 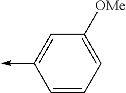 | | |
| | f | 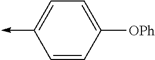 | | |

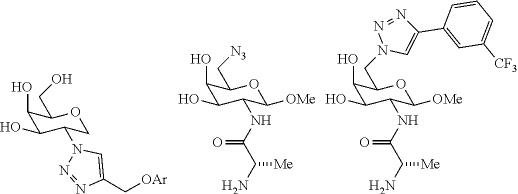

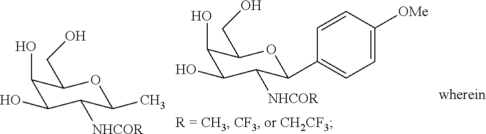

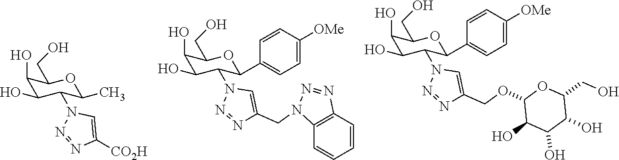

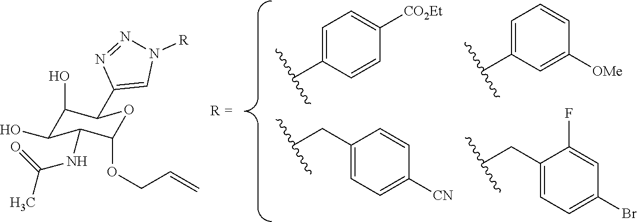

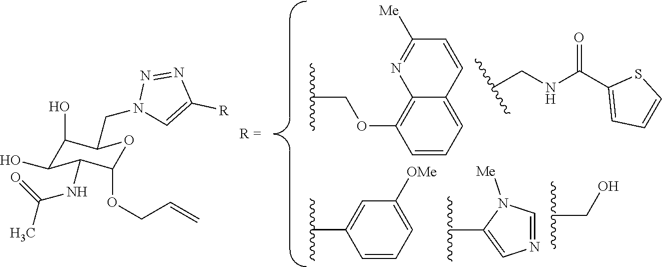

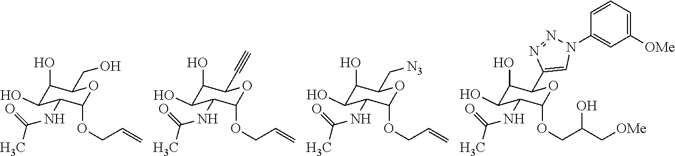 [SMILES: C=CCOC1OC(CO)C(O)C(O)C1NC(C)=O ; C#CC1OC(OCC=C)C(NC(C)=O)C(O)C1O ; C=CCOC1OC(CN=[N+]=[N-])C(O)C(O)C1NC(C)=O ; COCC(O)COC1OC(c2cn(-c3cccc(OC)c3)nn2)C(O)C(O)C1NC(C)=O]

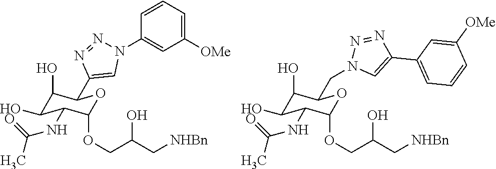

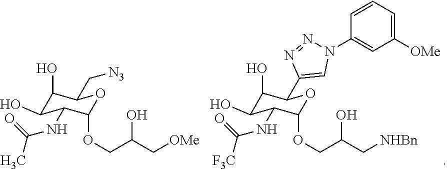

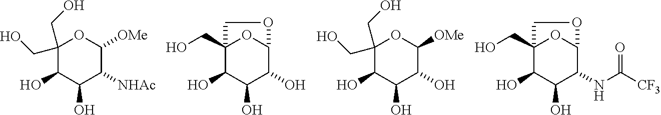 [SMILES: CO[C@H]1OC(CO)(CO)[C@H](O)[C@H](O)[C@H]1NC(C)=O ; OC[C@@]12CO[C@@H](O1)[C@H](O)[C@@H](O)[C@H]2O ; CO[C@@H]1OC(CO)(CO)[C@H](O)[C@H](O)[C@H]1O ; O=C(N[C@H]1[C@H]2OC[C@](CO)(O2)[C@H](O)[C@@H]1O)C(F)(F)F]

| | | R |
|---|---|---|
| 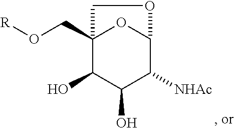 | 16a 16b 16c 16d 16e 16f | Et n-C₃H₇ n-C₄H₉ n-C₅H₁₁ n-C₆H₁₃ (CH₂CH₂O)₄Me |

[symbol]

indicates data missing or illegible when filed

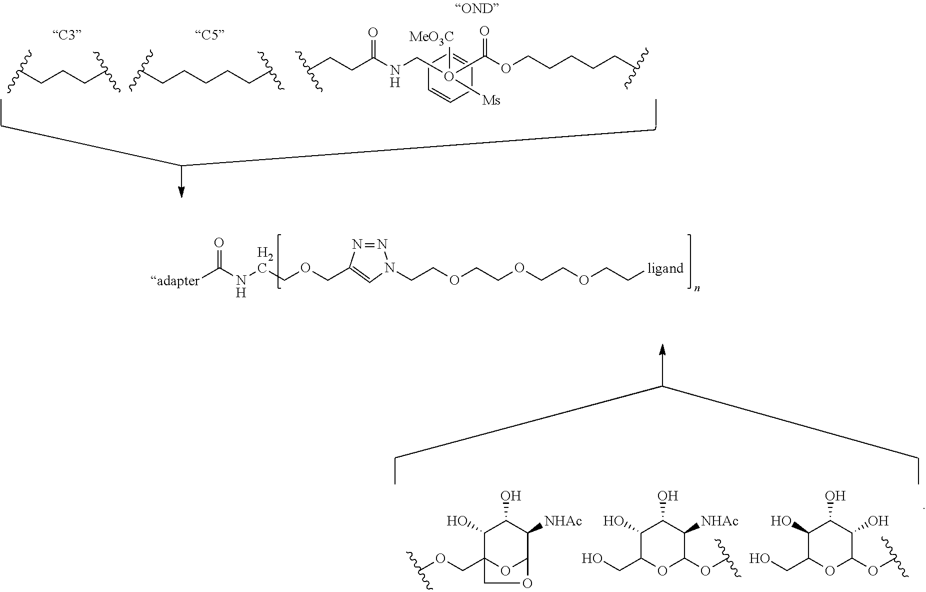

Embodiment 19 provides the compound of any one of Embodiments 1-18, wherein the Linker is a polyethylene glycol containing linker having 1-12 ethylene glycol residues.

Embodiment 20 provides the compound of any one of Embodiments 1-19, wherein the Linker comprises the structure:

—CH₂CH₂(OCH₂CH₂)ₘOCH₂—, —(CH₂)ₘCH₂—, —[N(R^{a})—CH(R^{b})(C═O)]ₘ—,
or a polypropylene glycol or polypropylene-co-polyethylene glycol group containing 1-100 alkylene glycol units;
   wherein each R^{a} is independently H, C₁-C₃ alkyl, or C₁-C₆ alkanol, or
   combines with R^{b} to form a pyrrolidine or hydroxypyrroline group;
   wherein each R^{b} is independently selected from the group consisting of hydrogen, methyl, isopropyl, —CH(CH₃)CH₂CH₃, —CH₂CH(CH₃)₂, —(CH₂)₃— guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
wherein m is an integer ranging from 1 to 15; or
   wherein the Linker comprises the structure

—[N(R′—(CH₂)₁₋₁₅—C(═O)]—,

wherein R′ is H or a C₁-C₃ alkyl optionally substituted with 1-2 hydroxyl groups, and

m is an integer ranging from 1 to 100; or

wherein the Linker comprises the structure:

-Z-D-Z′—,

wherein:
Z and Z′ are each independently a bond, —(CH₂)ᵢ—O—, —(CH₂)ᵢ—S—, —(CH₂)ᵢ—N(R)—,

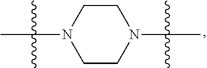 [SMILES: *N1CCN(*)CC1]

—(CH₂)ᵢ—C(R²)═C(R²)— (cis or trans), —(CH₂)ᵢ—≡—, or —Y—C(═O)—Y—;
each R is independently H, C₁-C₃ alkyl, or C₁-C₆ alkanol;
each R² is independently H or C₁-C₃ alkyl;
each Y is independently a bond, O, S, or N(R);
each i is independently 0 to 100;
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X¹ is O, S, or N(R);
j is an integer ranging from 1 to 100;
m′ is an integer ranging from 1 to 100;
n is an integer ranging from 1 to 100; or
   wherein the Linker comprises the structure:

—CH²—(OCH₂CH₂)ₙ—CH₂—, —(CH₂CH₂O)_{n′}CH₂CH₂—, or —(CH₂CH₂CH₂O)ₙ—,
wherein each n and n′ is independently an integer ranging from 1 to 25; or
   wherein the Linker comprises a structure:

-PEG-CON-PEG-
wherein each PEG is independently a polyethylene glycol group containing
from 1-12 ethylene glycol residues and CON is a triazole group

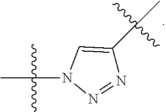 [SMILES: *c1cn(*)nn1]

Embodiment 21 provides the compound of any one of Embodiments 1-20,

wherein the CON comprises a structure:

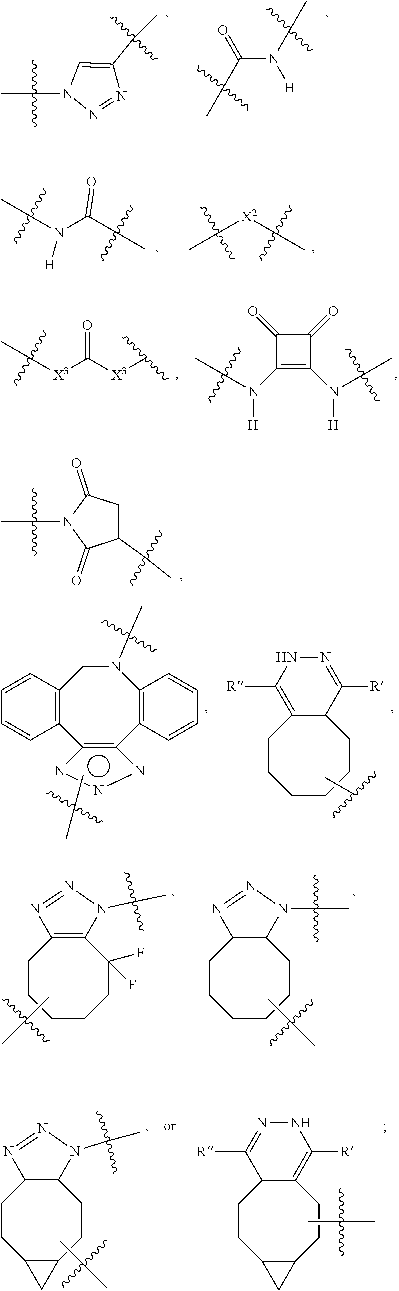

wherein R′ and R″ are each independently H, methyl, or a bond; or
wherein the CON comprises a structure:

—C(═O)—N(R¹)—(CH₂)_{n″}—N(R¹)C(═O)—,

—N(R¹)—C(═O)(CH₂)_{n″}—C(═O)N(R¹)—, or

—N(R¹)—C(═O)(CH₂)_{n″}—N(R¹)C(═O)—;
wherein each R¹ is independently H or C₁-C₃ alkyl, and n″ is independently an integer from 0 to 8, in certain embodiments 1 to 7, in certain embodiments 1, 2, 3, 4, 5 or 6; or
   wherein the CON comprises a structure:

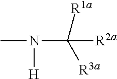

wherein:
R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}—, or —(CH₂)_{M2}O—(CH₂)_{M1}—C(O)NR⁴—, with the proviso that R^{1a}, R^{2a} and R^{3a} are not simultaneously H;
each M1 is independently 1, 2, 3, or 4;
each M2 is independently 0, 1, 2, 3, or 4;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0; or
   wherein the CON comprises a structure:

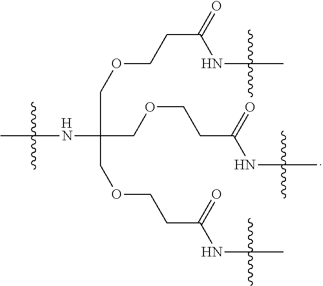

Embodiment 22 provides the compound of any one of Embodiments 1-21, wherein the extracellular protein comprises 1-40-O-amyloid, 5′-nucleotidase, activated F9, F10, activin receptor-like kinase 1, alpha-fetoprotein, amyloid, angiopoietin 2, angiopoietin 3, anthrax toxin, AOC3, AOC3 (VAP-1), Bacillus anthracis anthrax, BAFF, beta amyloid, c-Met, C1s, C242 antigen, C5, CA-125, calcitonin, calcitonin gene-related peptide, calcitonin gene-related peptide alpha, Canis lupus familiaris IL31, carbonic anhydrase 9 (CA-IX), CEA, CEA-related antigen, CEACAM5, CFD, CGRP, clumping factor A, coagulation factor III, complement C5a, CSF1, MCSF, CSF2, dabigatran, E. coli shiga toxin type-1, E. coli shiga toxin type-2, EGFL7, endotoxin, episialin, FGF 23, fibrin II, beta chain, fibronectin extra domain-B, folate hydrolase, GDF-8, gelatinase B, GMCSF, growth differentiation factor 8, hemagglutinin, hemagglutinin HA, HGF, HIV-1, HNGF, Hsp90, human beta-amyloid, human scatter factor receptor kinase, human TNF, IFN-α, IFN-γ, IgE, IgE Fc region, IGF1, IGF2, IGHE, IL 17A, IL 17A and IL 17F, IL 20, IL-1, IL-12, IL-23, IL-13, IL-17, IL-10, IL-22, IL-4, IL-5, IL-6, IL17A and IL17F, IL1A, IL2, IL23, IL23A, IL31RA, IL6, IL6R, IL9, ILGF2, Influenza A hemagglutinin, influenza A virus hemagglutinin, influenza A virus hemagglutinin HA, interferon gamma, interferon gamma-induced protein, interleukin 1 alpha, interleukin 13, interleukin 17 alpha, interleukin 17 alpha, TNF, interleukin 17A, kallikrein, LOXL2, LRRC15, LTA, MASP-2, MCP-1, MIF, MST1R (aka RON), MUC1, myostatin, NACP, NCA-90 (granulocyte antigen), neural apoptosis-regulated proteinase 1, NGF, NOGO-A, Notch 1, NRP1, oxLDL, PCSK9, PD-L1, phosphatidylserine, RANKL, RGMA, root plate-specific spondin 3, RTN4, sclerostin, SDC1, serum amyloid A protein, serum amyloid P component, SOST, Staphylococcus aureus alpha toxin, tau protein, TFPI, TGF beta 1, TGF beta 2, TGF-β, TNF-α, TROP-2, TSLP, VEGF-A, VEGF-A and Ang-2, VEGFA, or VWF.

Embodiment 23 provides the compound of any one of Embodiments 1-22, wherein the Ab is a monoclonal antibody.

Embodiment 24 provides the compound of any one of Embodiments 1-23, wherein the antibody comprises: Abagovomab, Abrezekimab, Adalimumab, Aducanumab, Afasevikumab, Afelimomab, Alirocumab, Altumomab, Altumomab pentetate, Andecaliximab, Anrukinzumab, Arcitumomab, Ascrinvacumab, Atezolizumab, Atidortoxumab, Atinumab, Avelumab, Bapineuzumab, Bavituximab, Belimumab, Bermekimab, Besilesomab, Bevacizumab, Biciromab, Bimekizumab, Birtamimab, Blosozumab, Bococizumab, Brazikumab, Briakinumab, Brodalumab, Brolucizumab, Brontictuzumab, Burosumab, Cabiralizumab, Canakinumab, Cantuzumab, Cantuzumab ravtansine, Caplacizumab, Carlumab, Cergutuzumab, Cergutuzumab amunaleukin, Certolizumab, Certolizumab pegol, Cibisatamab, Clazakizumab, Clivatuzumab, Clivatuzumab tetraxetan, Concizumab, Crenezumab, Dectrekumab, Denosumab, Dezamizumab, Diridavumab, Domagrozumab, Dorlimomab, Dorlimomab aritox, Durvalumab, Dusigitumab, Eculizumab, Edobacomab, Efungumab, Eldelumab, Elezanumab, Elsilimomab, Emactuzumab, Emapalumab, Emicizumab, Enokizumab, Epitumomab, Epitumomab cituxetan, Eptinezumab, Erenumab, Evinacumab, Evolocumab, Faricimab, Fasinumab, Fezakinumab, Ficlatuzumab, Firivumab, Fletikumab, Fontolizumab, Fremanezumab, Fresolimumab, Frovocimab, Frunevetmab, Fulranumab, Galcanezumab, Gantenerumab, Gatipotuzumab, Gedivumab, Gevokizumab, Gimsilumab, Girentuximab, Golimumab, Gosuranemab, Guselkumab, Idarucizumab, Igovomab, Imalumab, Indatuximab, Indatuximab ravtansine, Infliximab, Istiratumab, Ixekizumab, Labetuzumab, Lacnotuzumab, Lampalizumab, Lanadelumab, Landogrozumab, Lebrikizumab, Lemalesomab, Lendalizumab, Lenzilumab, Lerdelimumab, Lesofavumab, Ligelizumab, Lodelcizumab, Lokivetmab, Lutikizumab, Marstacimab, Mepolizumab, Metelimumab, Mirikizumab, Nacolomab, Nacolomab tafenatox, Namilumab, Narnatumab, Navivumab, Naxitamab, Nebacumab, Nemolizumab, NEOD, Nerelimomab, Nesvacumab, Netakimab, Nofetumomab, Nofetumomab merpentan, Obiltoxaximab, Oleclumab, Olendalizumab, Olokizumab, Omalizumab, OMS, Onartuzumab, Oregovomab, Orticumab, Otilimab, Ozanezumab, Ozoralizumab, Parsatuzumab, Pascolizumab, Pasotuxizumab, Pateclizumab, Pemtumomab, Perakizumab, Pexelizumab, Placulumab, Ponezumab, Prasinezumab, Pritoxaximab, Quilizumab, Radretumab, Ralpancizumab, Ranevetmab, Ranibizumab, Ravulizumab, Raxibacumab, REGN-EB, Remtolumab, Reslizumab, Rilotumumab, Risankizumab, Romilkimab, Romosozumab, Rontalizumab, Rosmantuzumab, Sacituzumab, Sacituzumab govitecan, Samrotamab, Samrotamab vedotin, Sarilumab, Secukinumab, Setoxaximab, Setrusumab, Sifalimumab, Siltuximab, Simtuzumab, Sirukumab, Sofituzumab, Sofituzumab, Sofituzumab vedotin, Solanezumab, Sontuzumab, Stamulumab, Sulesomab, Sutimlimab, Suvizumab, Suvratoxumab, Tabalumab, Tacatuzumab, Tacatuzumab tetraxetan, Talizumab, Tanezumab, Tefibazumab, Telimomab, Telimomab aritox, Tesidolumab, Tezepelumab, Tibulizumab, Tildrakizumab, Timolumab, Tisotumab, Tisotumab vedotin, Tralokinumab, Trevogrumab, Urtoxazumab, Ustekinumab, Vanucizumab, Vapaliximab, Varisacumab, Vepalimomab, Vesencumab, Vobarilizumab, Vunakizumab, or Xentuzumab.

Embodiment 25 provides a pharmaceutical composition comprising at least one pharmaceutically acceptable excipient and at least one compound of any one of Embodiments 1-24.

Embodiment 26 provides the pharmaceutical composition of Embodiment 25, further comprising another therapeutically active compound.

Embodiment 27 provides a compound selected from the group consisting of:

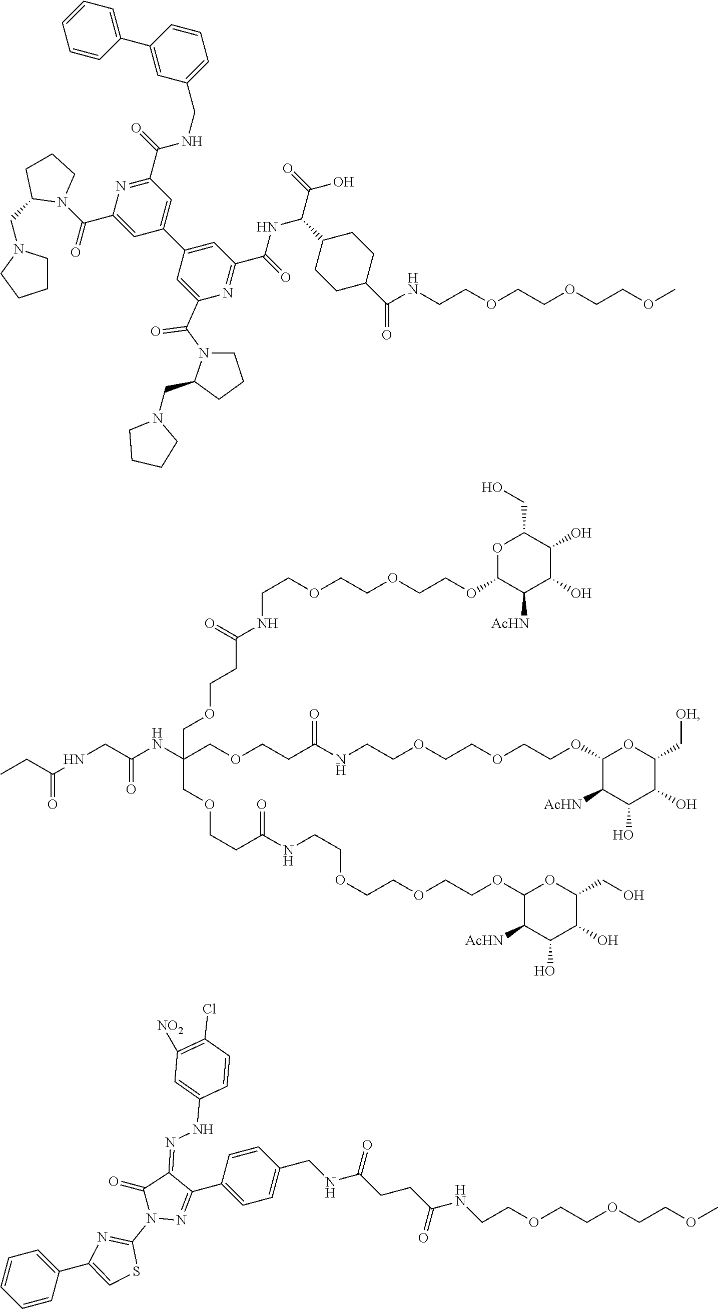

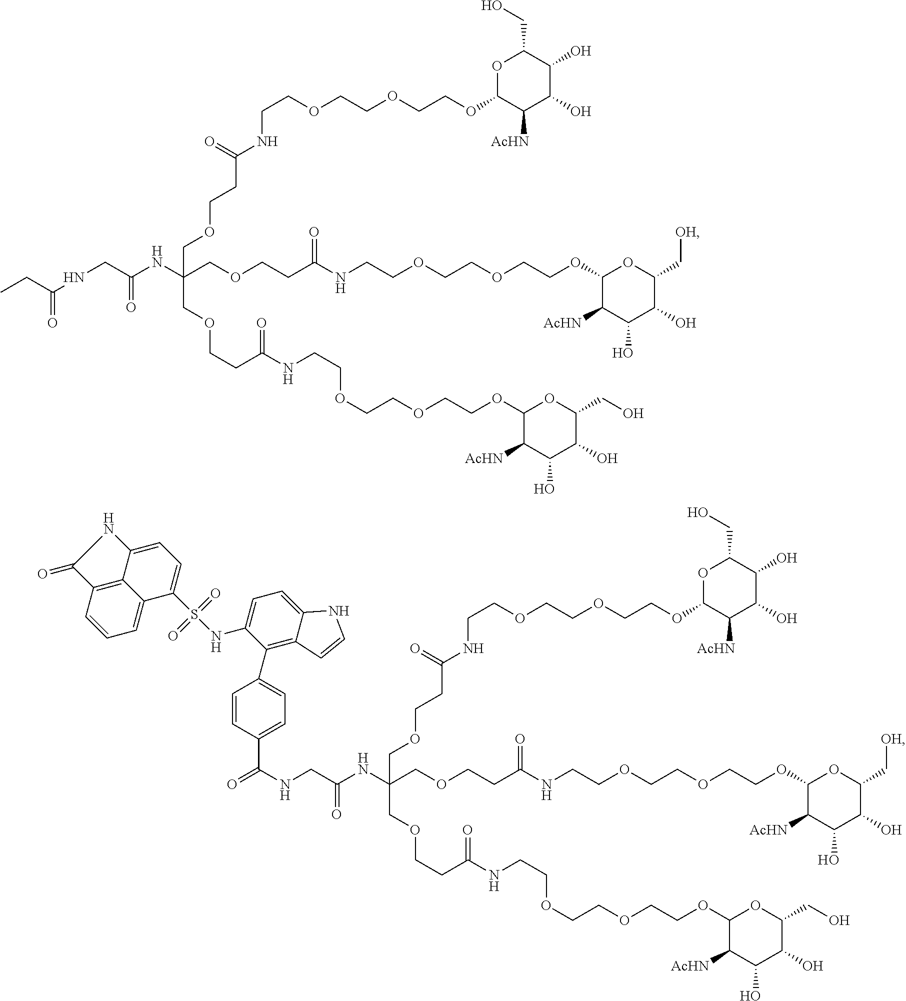 [SMILES: CCC(=O)NCC(=O)NC(COCCC(=O)NCCOCCOCCOC1O[C@H](CO)[C@H](O)[C@H](O)[C@H]1NC(C)=O)(COCCC(=O)NCCOCCOCCO[C@@H]1O[C@H](CO)[C@H](O)[C@H](O)[C@H]1NC(C)=O)COCCC(=O)NCCOCCOCCO[C@@H]1O[C@H](CO)[C@H](O)[C@H](O)[C@H]1NC(C)=O ; CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)c2ccc(-c3c(NS(=O)(=O)c4ccc5c6c(cccc46)C(=O)N5)ccc4[nH]ccc34)cc2)O[C@H](CO)[C@H](O)[C@@H]1O]

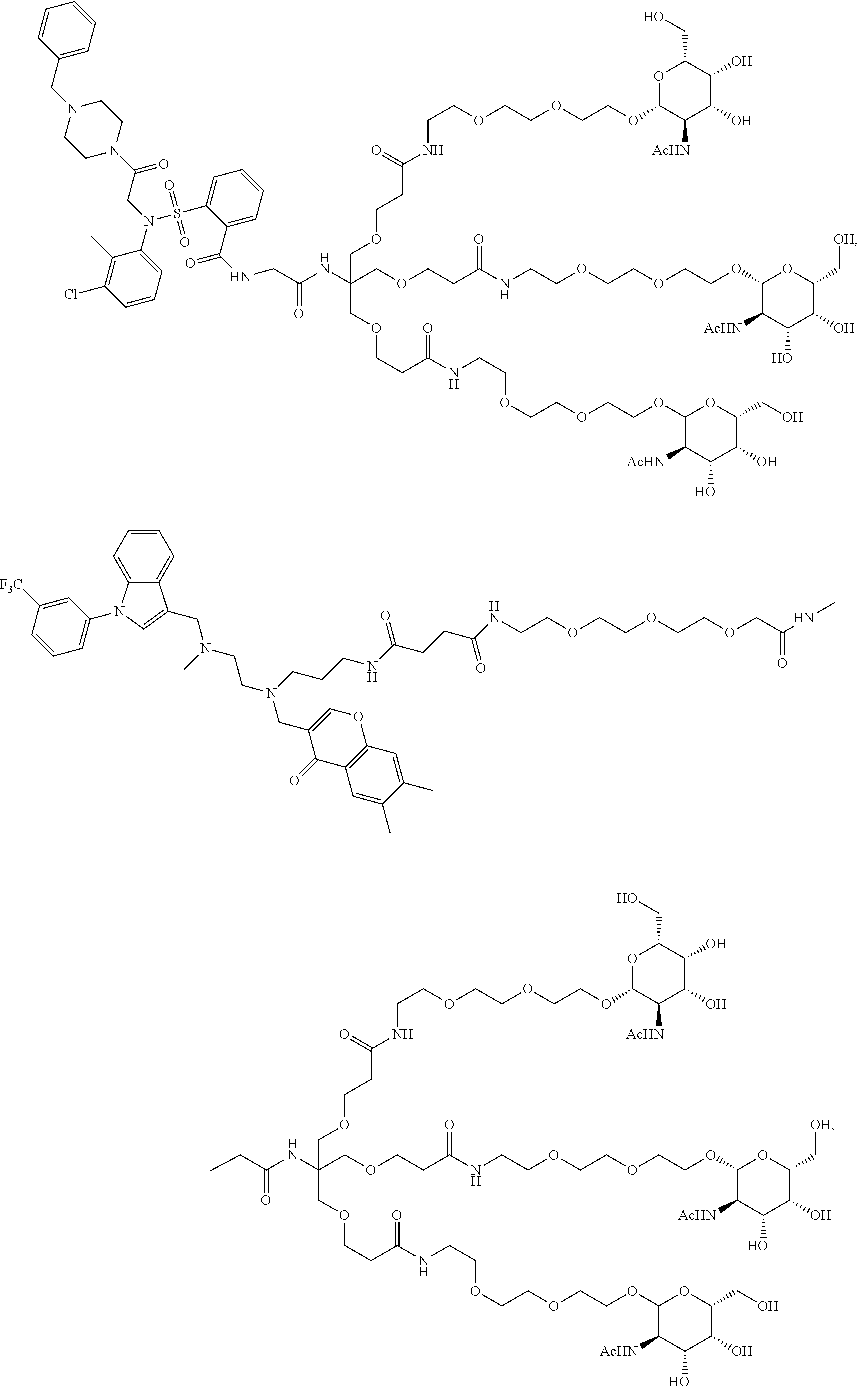

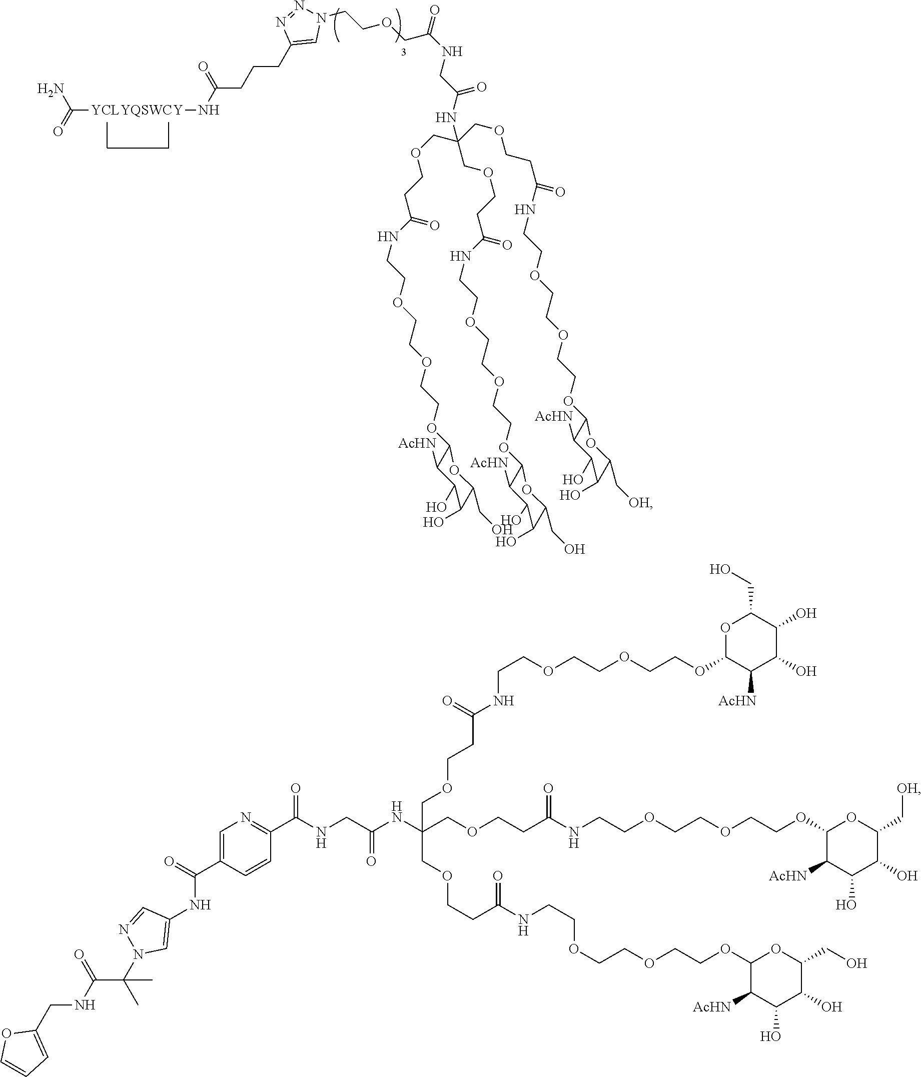

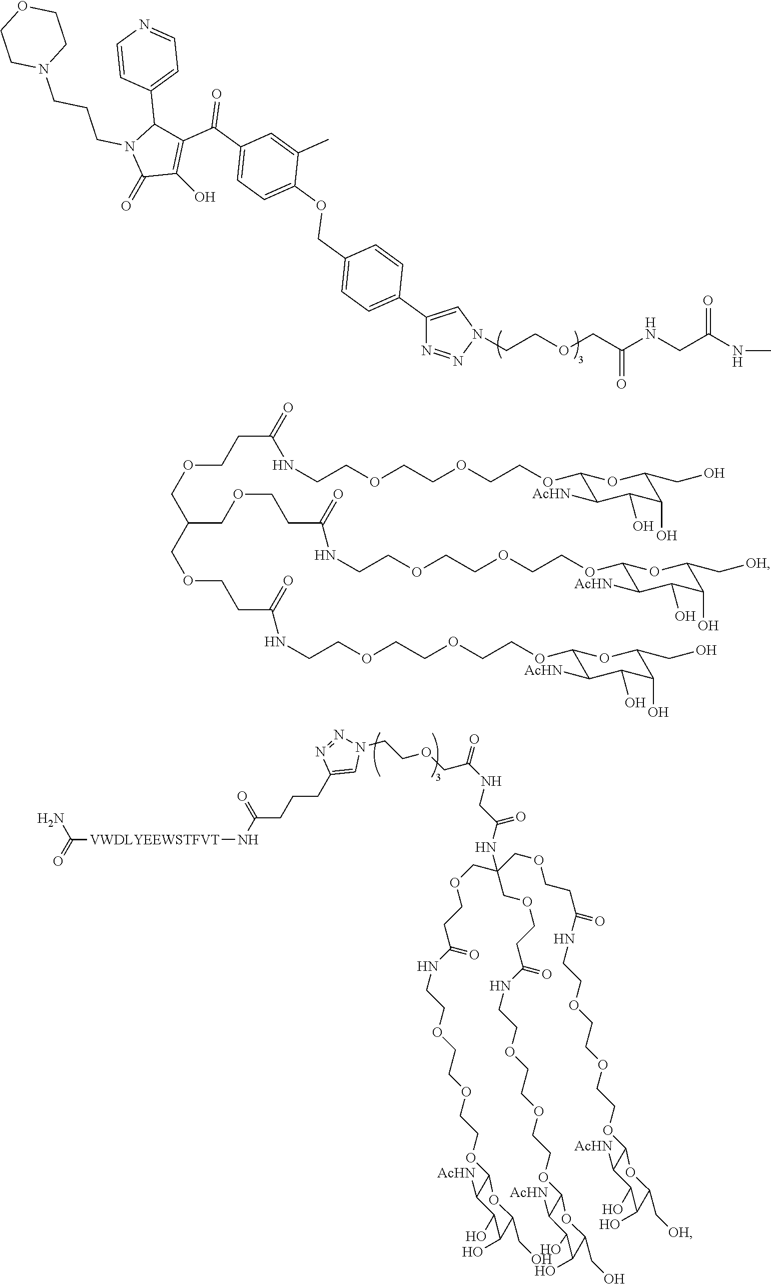

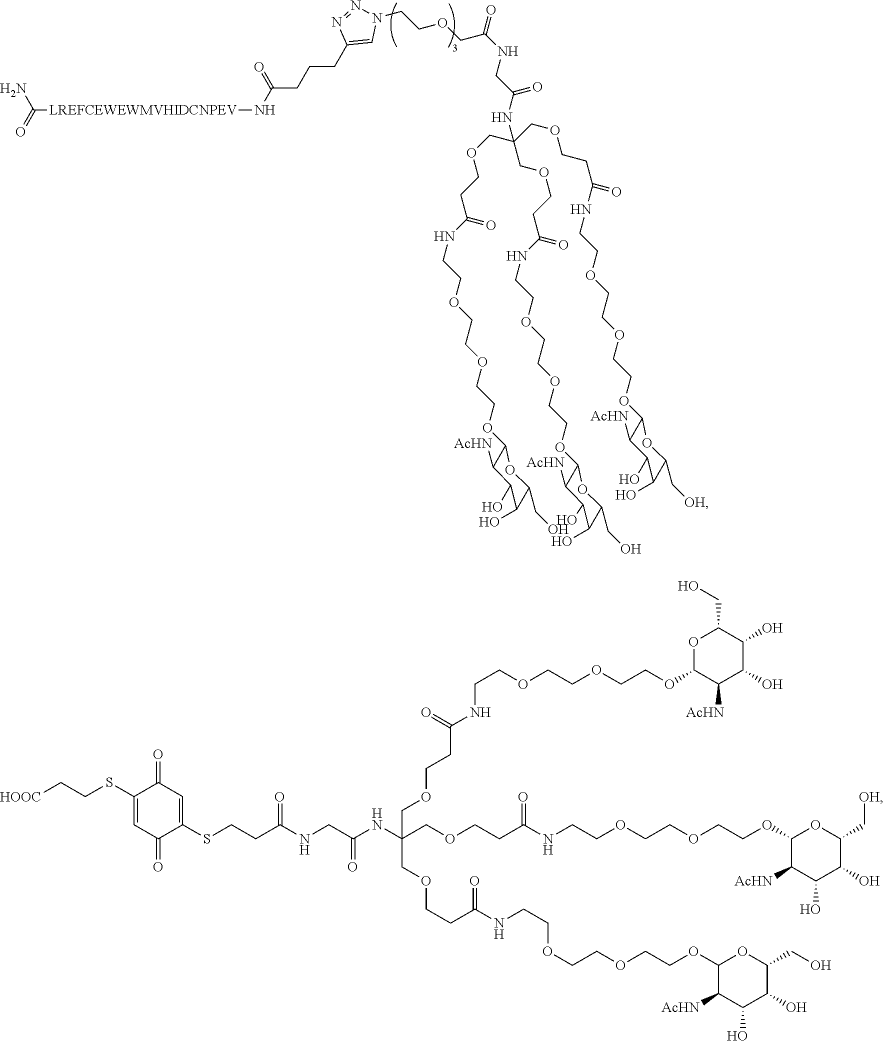

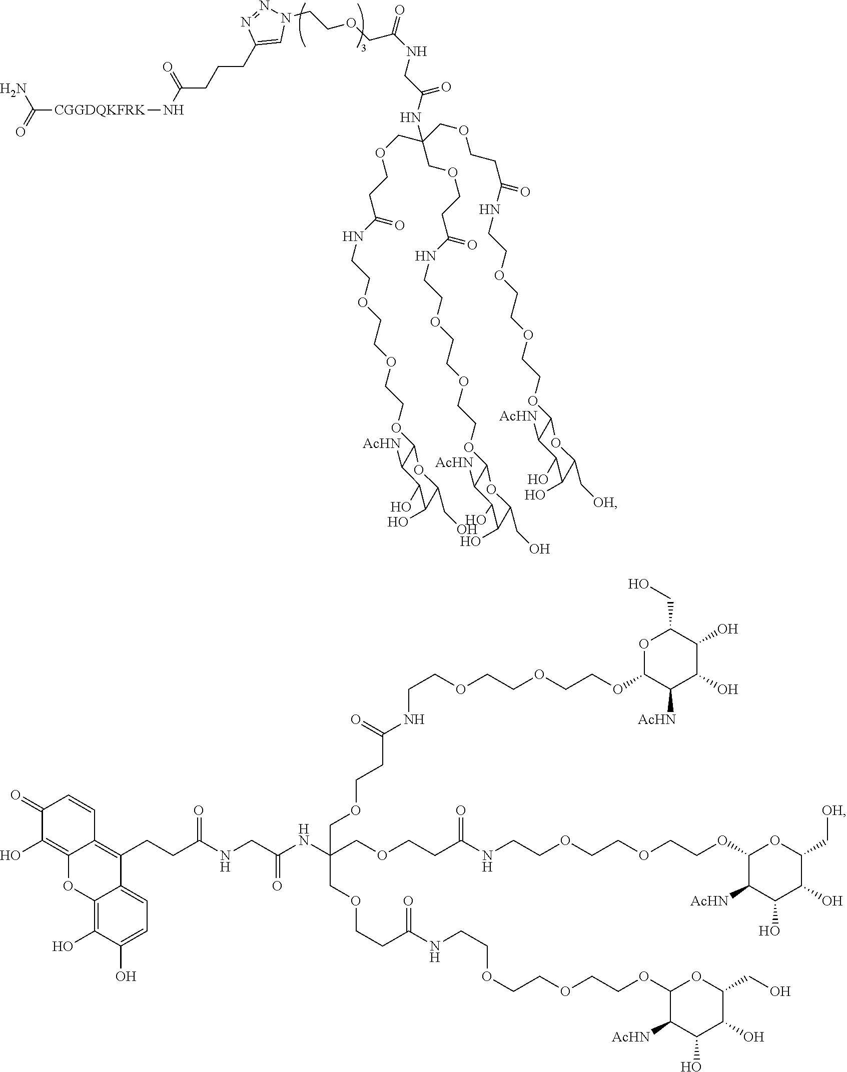

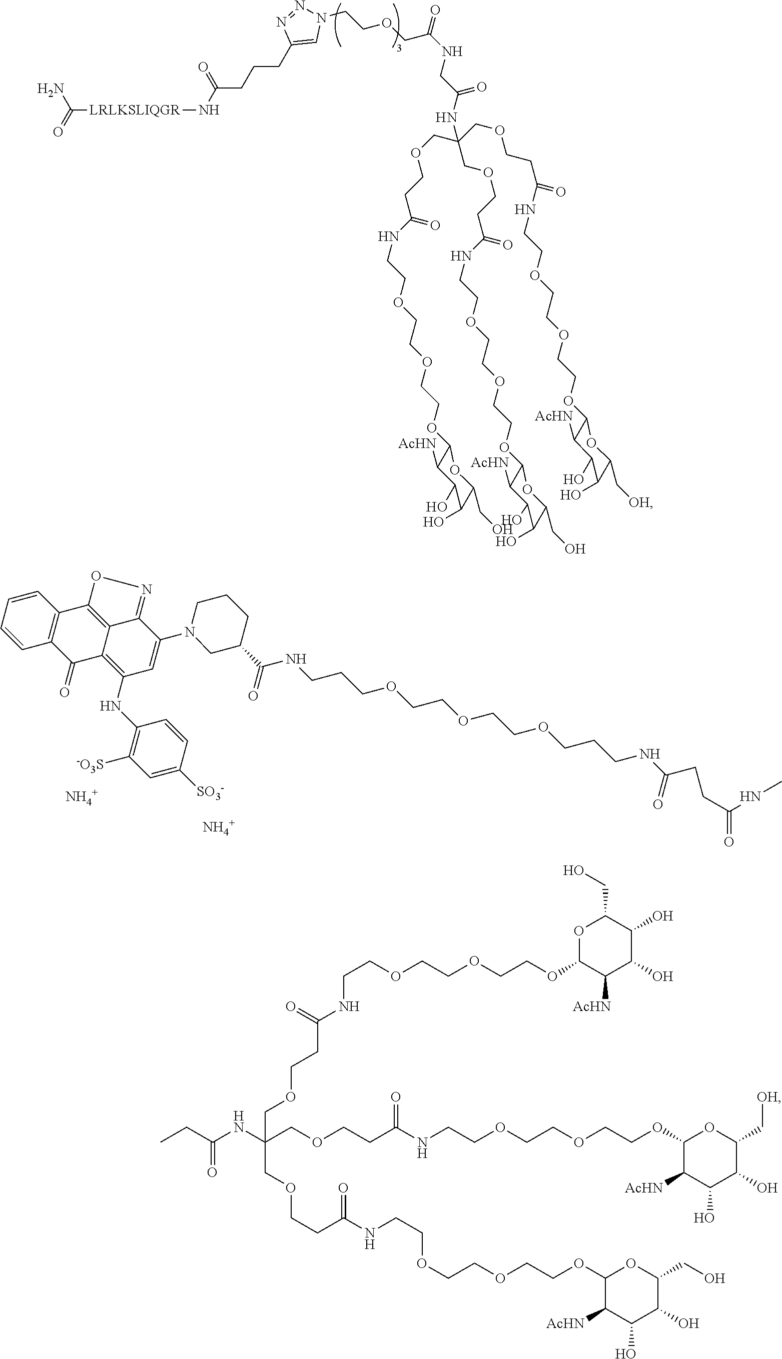

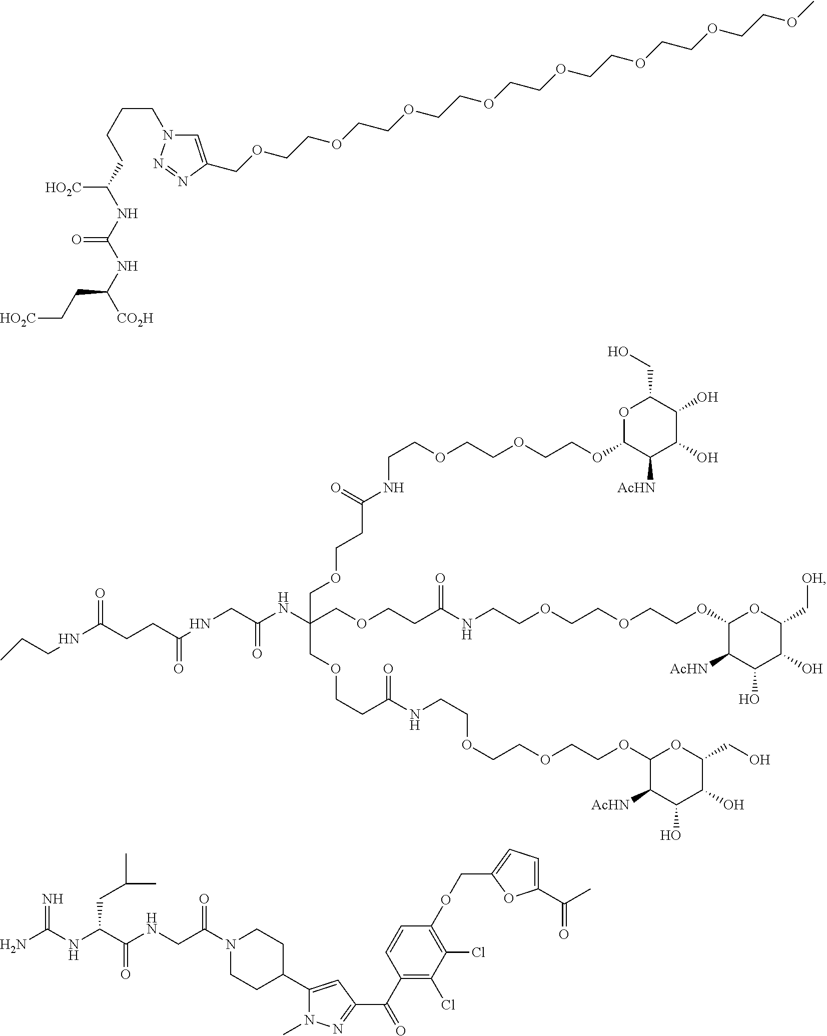

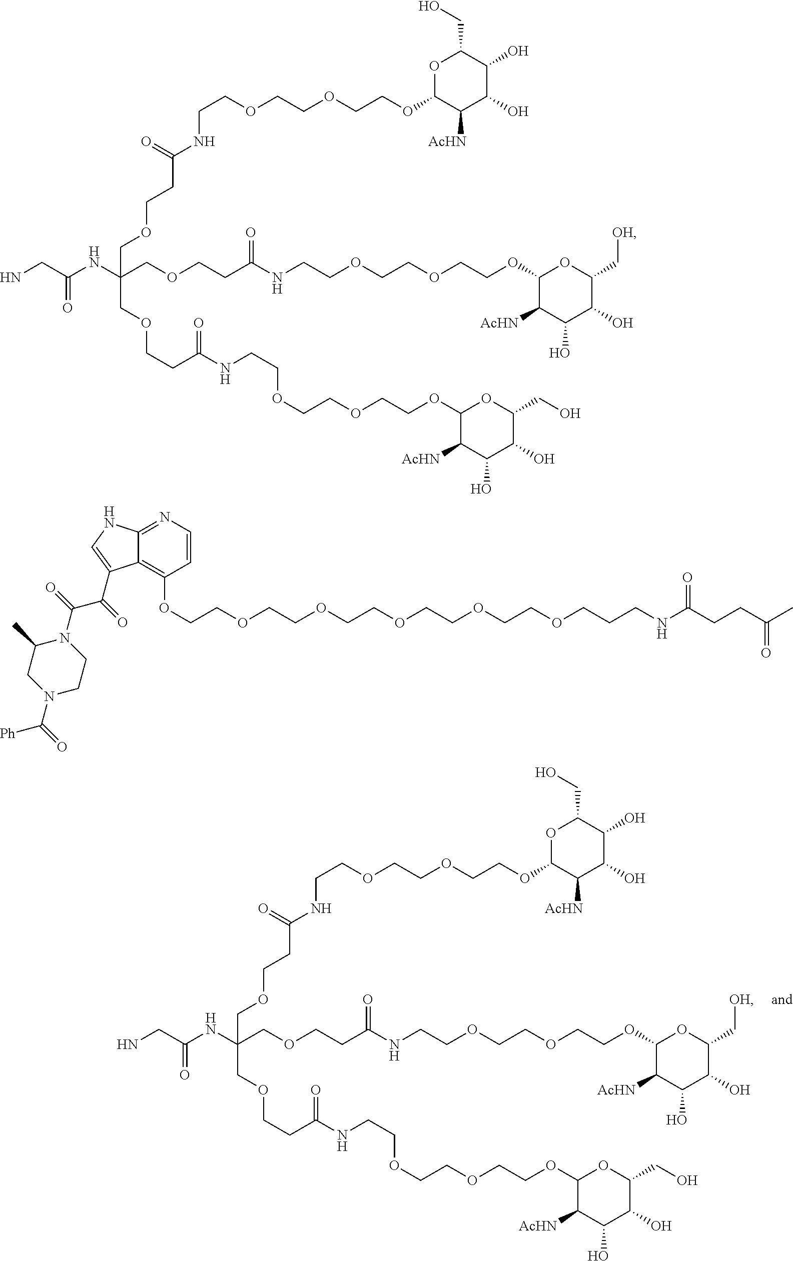

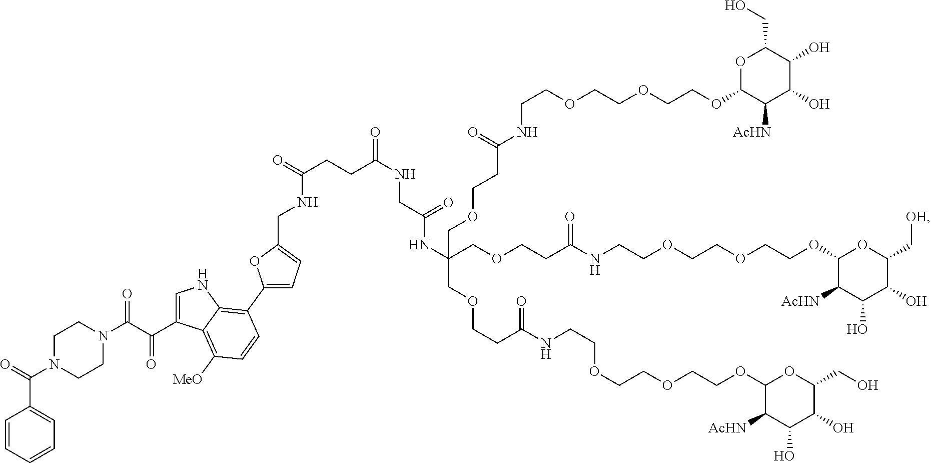 [SMILES: COc1ccc(-c2ccc(CNC(=O)CCC(=O)NCC(=O)NC(COCCC(=O)NCCOCCOCCOC3O[C@H](CO)[C@H](O)[C@H](O)[C@H]3NC(C)=O)(COCCC(=O)NCCOCCOCCO[C@@H]3O[C@H](CO)[C@H](O)[C@H](O)[C@H]3NC(C)=O)COCCC(=O)NCCOCCOCCO[C@@H]3O[C@H](CO)[C@H](O)[C@H](O)[C@H]3NC(C)=O)o2)c2[nH]cc(C(=O)C(=O)N3CCN(C(=O)c4ccccc4)CC3)c12]

or a salt, stereoisomer, or solvate thereof.

Embodiment 28 provides the compound of any one of Embodiments 1-27, having one of the following formulae:

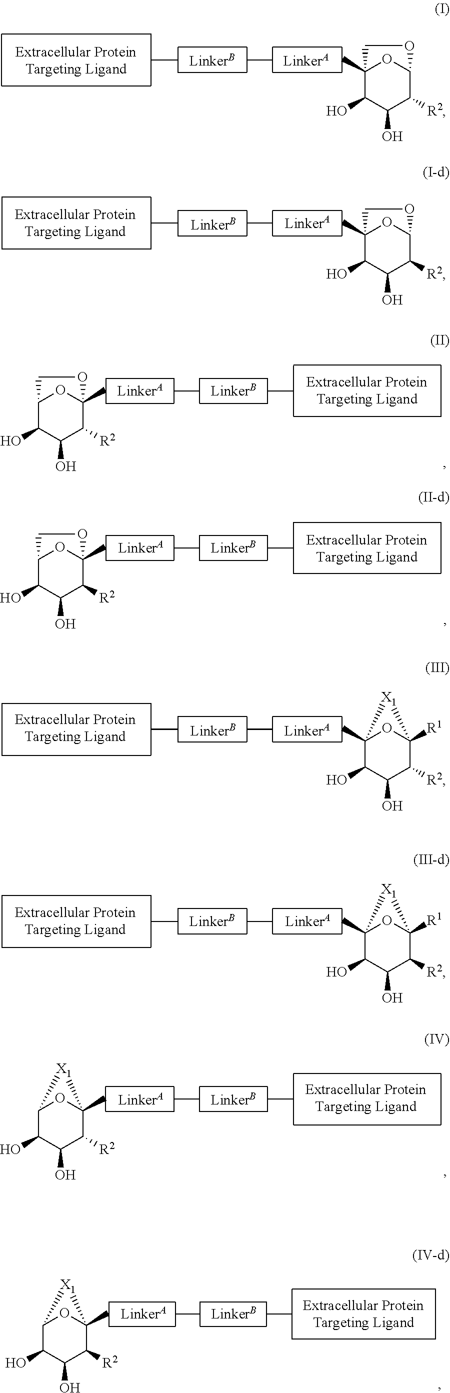

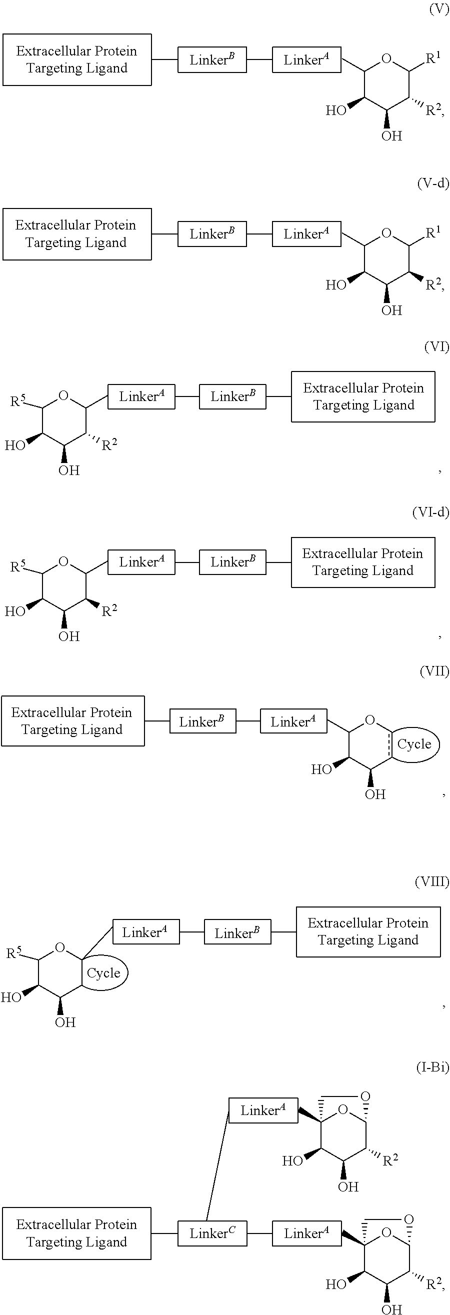

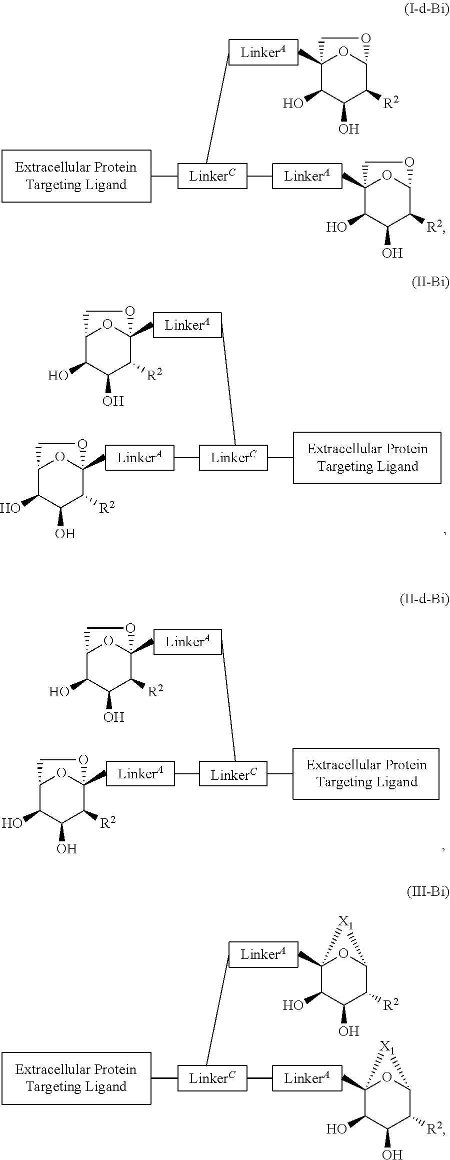

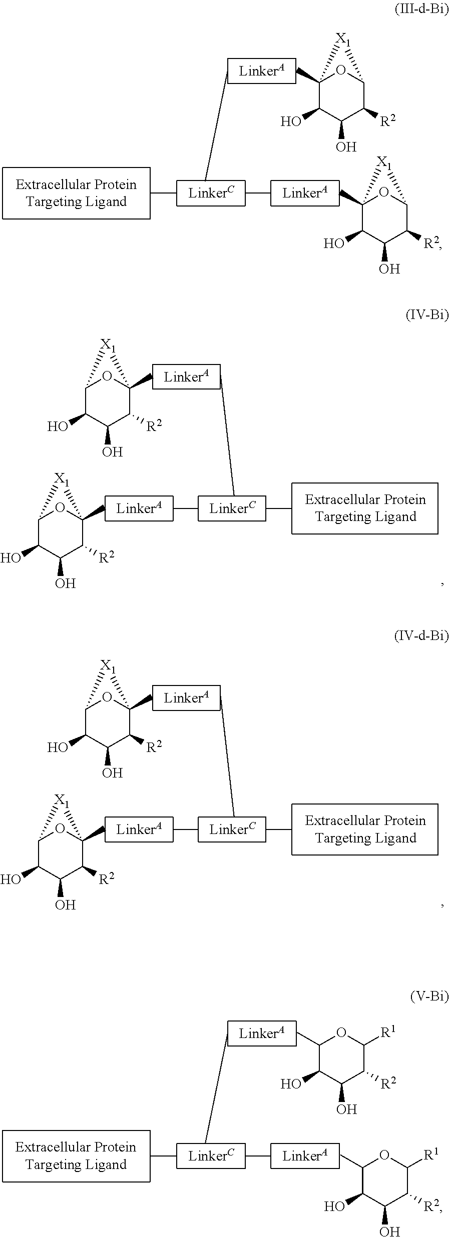

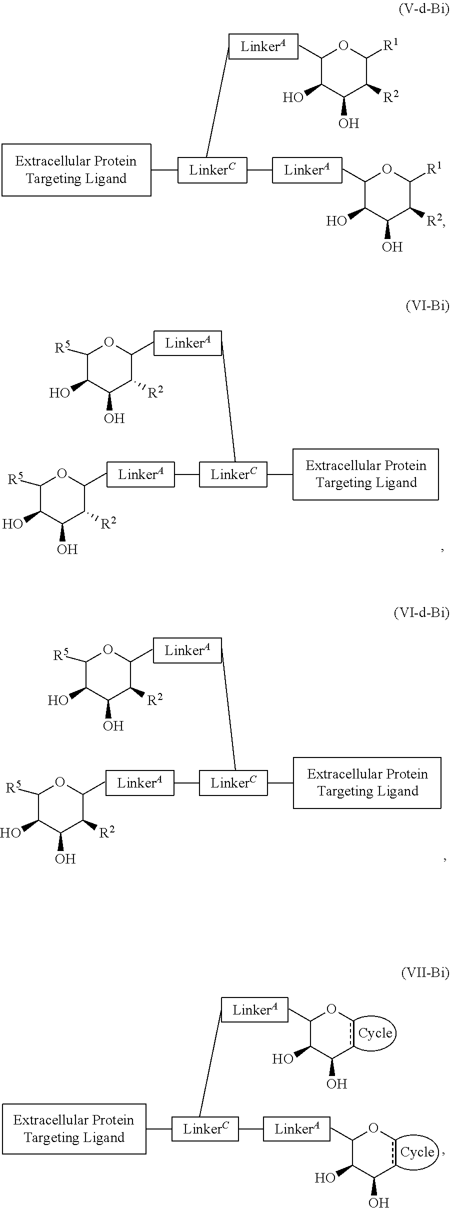

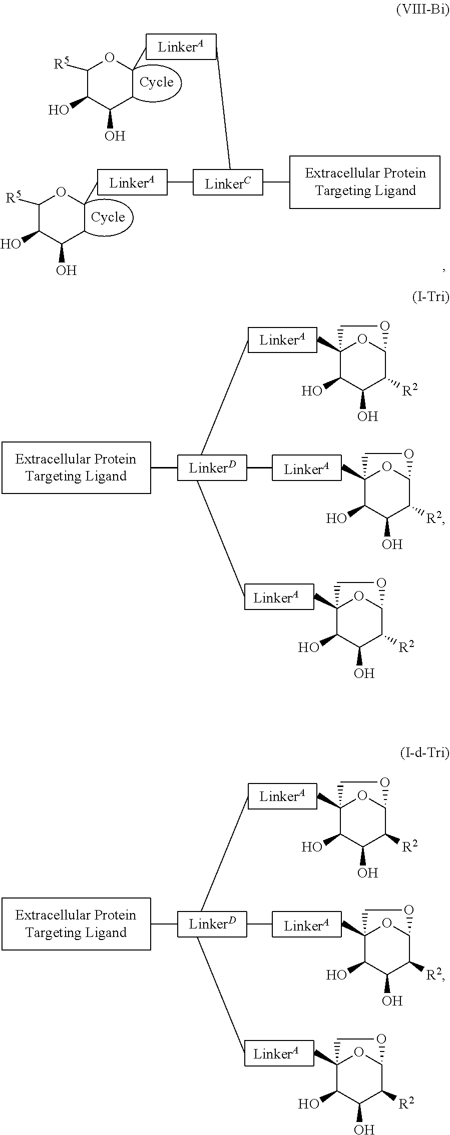

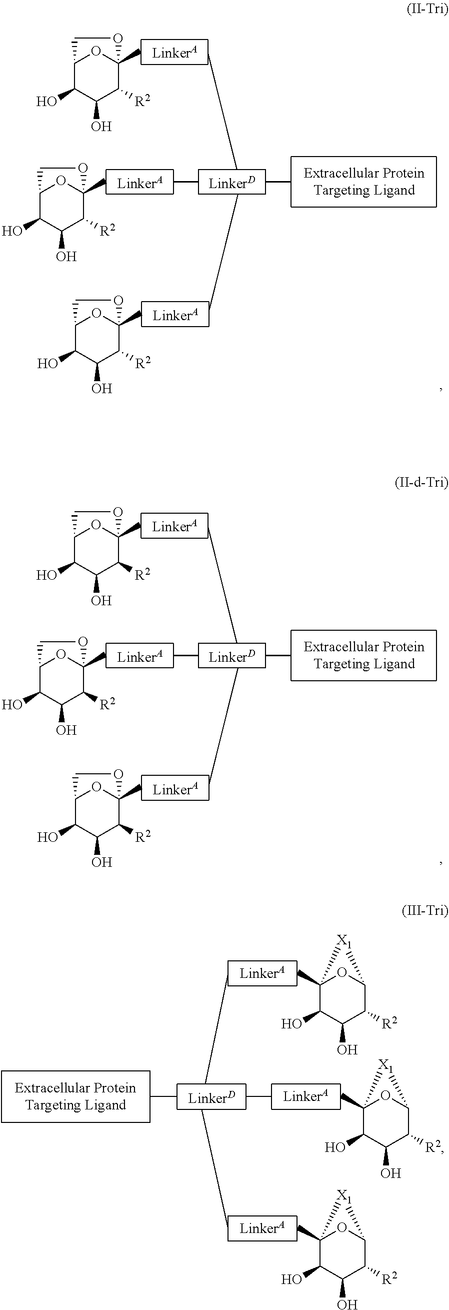

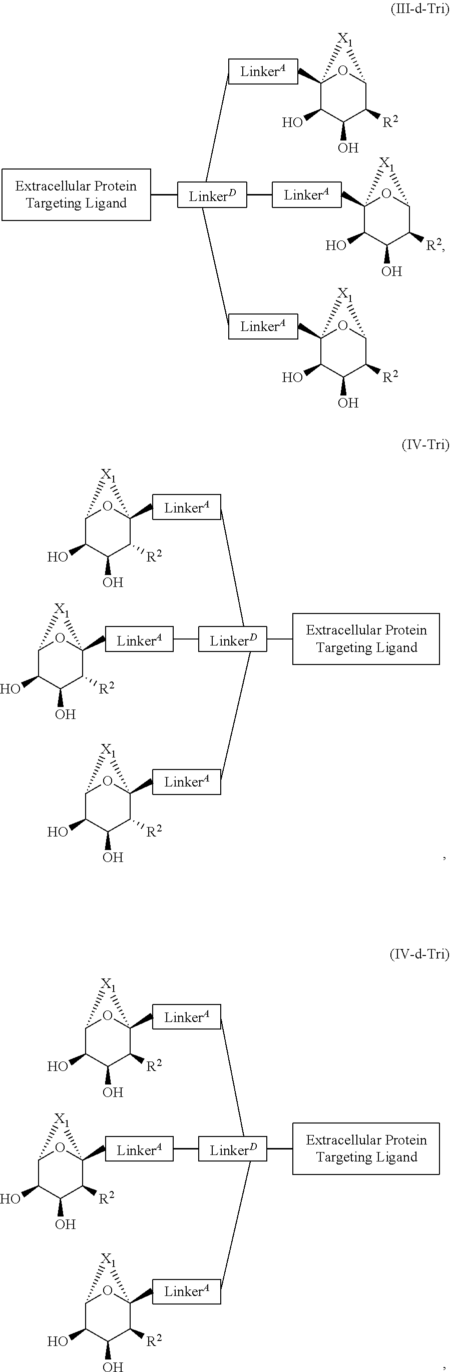

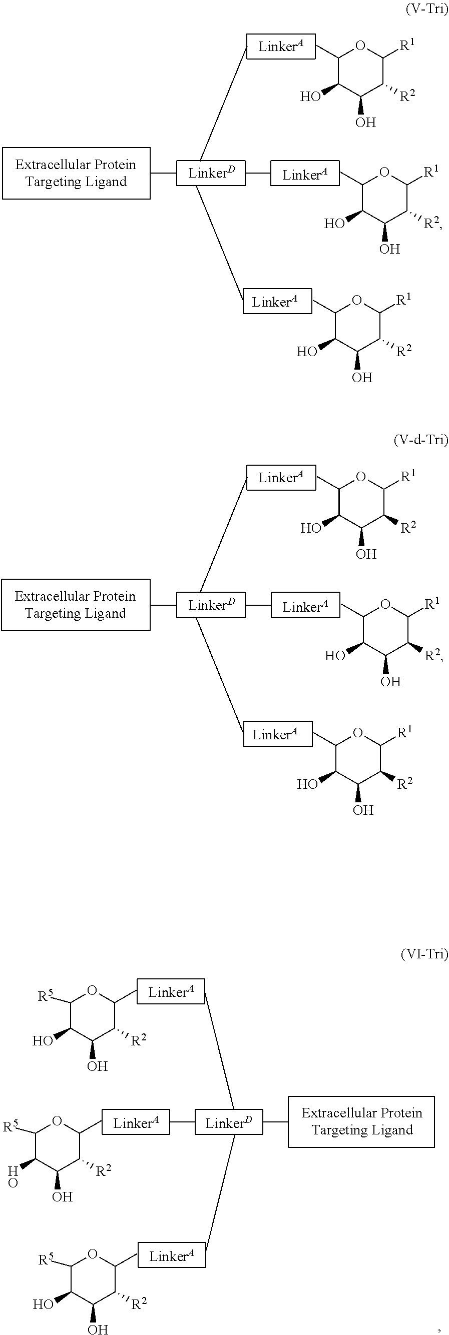

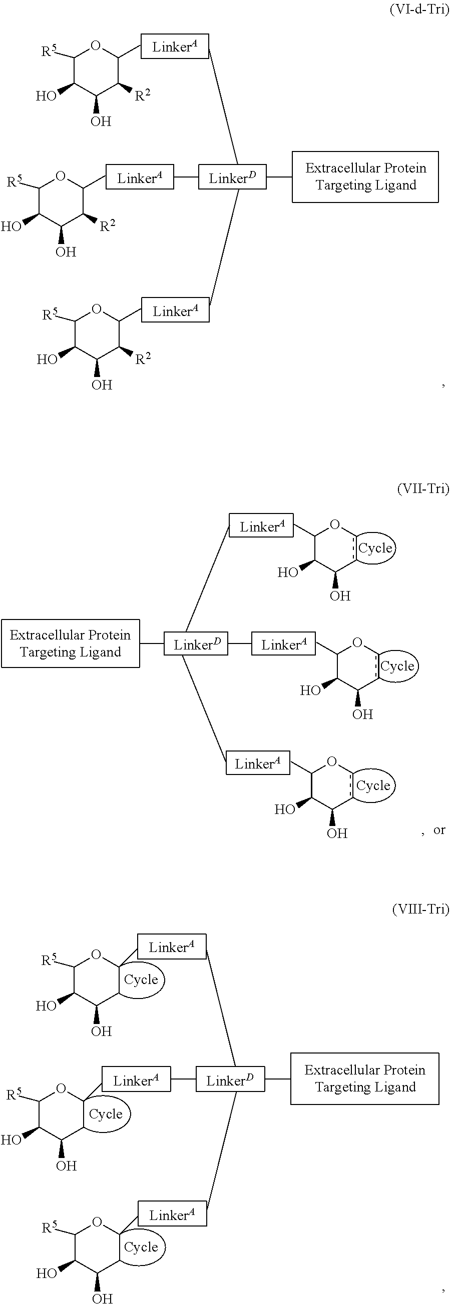

or a pharmaceutically acceptable salt thereof

wherein:

Extracellular Protein Targeting Ligand is a Circulating Protein Binding Moiety [CPBM] which binds to a circulating protein in a subject, wherein the circulating protein mediates a disease state or condition and is to be removed by the action of hepatocytes or other cells of the subject;

X¹ is 1 to 5 groups independently selected from O, S, N(R⁶), and C(R⁴)(R⁴), wherein if X¹ is 1 group then X¹ is O, S, N(R⁶), or C(R⁴)(R⁴), if X¹ is 2 groups then n more than 1 group of X¹ is O, S, or N(R⁶), if X¹ is 3, 4, or 5 groups then no more than 2 groups of X¹ are O, S, or N(R⁶);

R² is selected from
(i) aryl, heterocycle, and heteroaryl containing 1 or 2 heteroatoms independently selected from N, O, and S, each of which aryl, heterocycle, and heteroaryl is optionally substituted with 1, 2, 3, or 4 substituents,

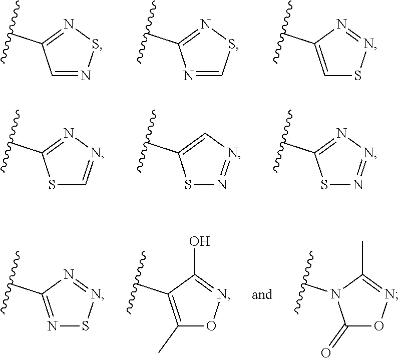

(iii) —NR⁸—S(O)—R³, —NR⁸—C(S)—R³, —NR⁸—S(O)(NR⁶)—R³, —N═S(O)(R³)₂, —NR⁸(O)NR⁹S(O)₂R¹⁰, —NR⁸—S(O)₂—R¹⁰, and —NR⁸—C(NR⁶)—R³ each of which is optionally substituted with 1, 2, 3, or 4 substituents, and
(iv) hydrogen, R¹⁰, alkyl-C(O)—R³, —C(O)—R³, alkyl, haloalkyl, —OC(O)R³, and —NR⁸—C(O)R¹⁰;

R¹⁰ is selected from aryl, alkyl-NR⁸—C(O)—R³, alkyl-aryl, alkyl-heteroaryl with 1, 2, or 4 heteroatoms, alkyl-cyano, alkyl-OR⁶, alkyl-NR⁶R⁸, NR⁸—NR⁶—C(O)R³, NR⁸—S(O)₂—R³, alkenyl, allyl, alkynyl, —NR⁶-alkenyl, —O-alkenyl, —NR⁶-alkynyl, —NR⁶-heteroaryl, —NR⁶-aryl, —O-heteroaryl, —O-aryl and —O-alkynyl, each of which R¹⁰ is optionally substituted with 1, 2, 3, or 4 substituents;

R¹ and R⁵ are independently selected from hydrogen, heteroalkyl, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, —O-alkenyl, —O-alkynyl, C₀-C₆alkyl-OR⁶, C₀-C6alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆akyl-N(R⁸)—S(O)R³, C₀-C₆alkyl-N(R⁸)—C(S)R³, C₀-C6alkyl-N(R⁸)S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents;

R³ at each occurrence is independently selected from hydrogen, alkyl, heteroalkyl, haloalkyl (including —CF₃, —CHF₂, —CH₂F, —CH₂CF₃, —CH₂CH₂F, and —CF₂CF₃), arylalkyl, heteroarylalkyl, alkenyl, alkynyl, aryl, heteroaryl, heterocycle, —OR⁸, and —NR⁸R⁶;

R⁴ is independently selected at each occurrence from hydrogen, heteroalkyl, alkyl, haloalkyl, arylalkyl, heteroarylalkyl, alkenyl, alkynyl, aryl, heteroaryl, heterocycle, -OR⁶, —NR⁶R⁷, C(O)R³, S(O)R³, C(S)R³, and S(O)₂R³,

R⁸ and R⁹ are independently selected at each occurrence from hydrogen, heteroalkyl, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;

R⁸ and R⁹ are independently selected at each occurrence from hydrogen, heteroalkyl, alkyl, arylalkyl, heteroarylalkyl, alkenyl, alkynyl, aryl, heteroaryl, and heterocycle;

Cycle is a 3-8 membered fused cyclic group optionally substituted with 1, 2, 3, or 4 substituents;

each Linker^{A} is a bond or a moiety that covalently links the ASGPR ligand to Linker^{B};

Linker^{B} is a bond or a moiety that covalently links Linker^{A} to an Extracellular Protein Targeting Ligand;
Linker^{C} is a chemical group that links each Linker^{A} to the Extracellular Protein Targeting Ligand; and
Linker^{D} is a chemical group that links each Linker^{A} to the Extracellular Protein Targeting Ligand; and
wherein, when R² is NR⁶-alkenyl, —NR⁶-alkynyl, —NR⁸—C(O)R¹⁰, —NR⁸—S(O)₂-alkenyl, —NR⁸—S(O)₂alkynyl, —NR⁶-heteroaryl, or —NR⁶-aryl, then Extracellular Protein Targeting Ligand does not comprise oligonucleotide; and
the optional substituents are selected from alkyl, alkenyl, alkynyl, haloalkyl, —OR⁶, F, Cl, Br, I, —NR⁶R⁷, heteroalkyl, cyano, nitro, C(O)R³,

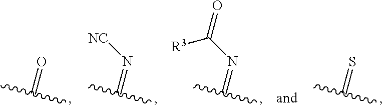

as allowed by valence such that a stable compound results.

Embodiment 29 provides a method of treating, ameliorating, and/or preventing a disease or disorder in a subject, the method comprising administering a therapeutically effective amount of at least one compound of any one of Embodiments 1-24 and 27-29 and/or at least one pharmaceutical composition of any one of Embodiments 25-26.

Embodiment 30 provides the method of Embodiment 29, wherein the disease or disorder comprises an autoimmune disease, cancer, or inflammation.

Embodiment 31 provides the method of Embodiment 30, wherein the autoimmune disease comprises Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (AP1S), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, esosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymyositis, acute disseminated encephalomyelitis (ADEM), acute motor axonic neuropathy, anti-NMDA receptor encephalitis, Balo concentric sclerosis, Bickerstaffs encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, or pyoderma gangrenosum.

Embodiment 32 provides the method of Embodiment 30, wherein the cancer comprises prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer, or lymphoma.

Embodiment 33 provides the method of Embodiment 30, wherein the inflammation comprises inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, or arteriosclerosis.

Embodiment 34 provides the method of any one of Embodiments 29-33, wherein the subject is further administered at least one additional therapeutic agent that treats or prevents the disease or disorder.

Embodiment 35 provides the method of any one of Embodiments 29-34, wherein the subject is a mammal.

Embodiment 36 provides the method of any one of Embodiments 29-35, wherein the subject is a human.

Embodiment 37 provides a method of treating, preventing, or preventing an autoimmune disease, cancer, or inflammation, the method comprising administering a composition comprising a therapeutically effective amount of at least one compound of claim 27 or 28 and at least one pharmaceutically acceptable carrier or excipient, wherein the autoimmune disease comprises Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (AP1S), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, esosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymysositis, acute disseminated encephalomyelitis (ADEM), acute motor axonic neuropathy, anti-NMDA receptor encephalitis, Balo concentric sclerosis, Bickerstaff's encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, or pyoderma gangrenosum;

wherein the cancer comprises prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer, or lymphoma; and

wherein the inflammation comprises inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, or arteriosclerosis.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A compound comprising formula (I), or a salt, geometric isomer, stereoisomer, or solvate thereof:
[Ab]_{k′}—[CON]ₕ-[Linker]ᵢ-[CON]_{h′}—[CRBM]_{j′} (I),
wherein:
the Ab is an antibody that binds to an extracellular protein;
the CRBM is a cellular receptor binding moiety that binds to at least one receptor on the surface of a degrading cell in a subject, whereby binding of (I) leads to endocytosis and degradation of the extracellular protein;
each CON is independently a bond or a group that covalently links an Ab to an CRBM, an Ab to a Linker, and/or a Linker to a CRBM;
the Linker is a group having a valence ranging from 1 to 15;
k′ is an integer ranging from 1 to 15;
h is an integer ranging from 0 to 15;
i is an integer ranging from 0 to 15;
h′ is an integer ranging from 0 to 15;
j is an integer ranging from 1 to 15.

2. The compound of claim 1, wherein the valence of the Linker is 1, 2, or 3.

3. The compound of claim 1, wherein k′ is 1, 2, or 3.

4. The compound of claim 1, wherein j is 1, 2, or 3.

5. The compound of claim 1, wherein his 1, 2, or 3.

6. The compound of claim 1, wherein h′ is 1, 2, or 3.

7. The compound of claim 1, wherein i is 1, 2, or 3.

8. The compound of claim 1, wherein at least one of h, h′, and i is at least 1.

9. The compound of claim 1, wherein k′, j′, h, h′, and i are each independently 1, 2, or 3.

10. The compound of claim 1, wherein k′ is 1, and j′ is 1, 2, or 3.

11. The compound of claim 1, which is:
[Ab]-[CON]₀₋₁-[Linker]-[CON]₀₋₁—[CRBM] (Ia).

12. The compound of claim 1, wherein the degrading cell comprises a hepatocyte.

13. The compound of claim 1, wherein the CRBM is a folic acid (folate) receptor binder, mannose receptor binder, mannose-6-phosphate (M6P) receptor binder, low density lipoprotein receptor-related protein 1 (LRP1) receptor binder, low density lipoprotein receptor (LDLR) binder, FcγRI receptor binder, transferrin receptor binder, macrophage scavenger receptor binder, G-Protein coupled receptor binder, or asialoglycoprotein receptor (ASGPR) binder.

14. The compound of claim 1, wherein the CRBM is:
(a) a folic acid (folate) receptor binder comprising at least one of folic acid, methotrexate, premetrexed, or a biologically active fragment thereof;
(b) a mannose receptor binder comprising at least one of: 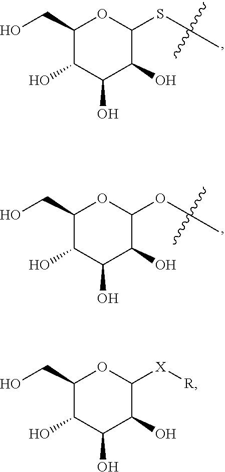
wherein: X is S or O, R is selected from the group consisting of: 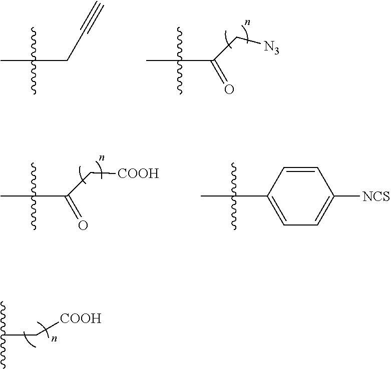
and each occurrence of ‘n’ is independently 1, 2, 3, 4, 5, 6, 7, 8, 9, 10, 11, 12, 13, 14, 15, 16, 17, 18, 19, or 20; and
a polymeric molecule illustrated in FIGS. 1-7;
(c) a mannose-6-phosphate (M6P) receptor binder comprising at least one of: 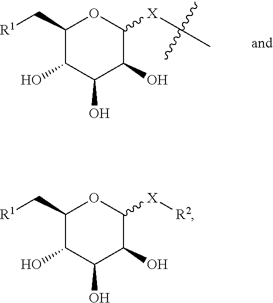
wherein X is O or S, R¹ is selected from the group consisting of: 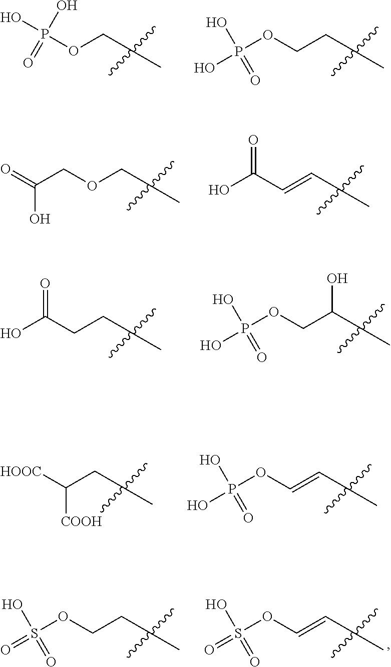
R² is selected from the group consisting of: 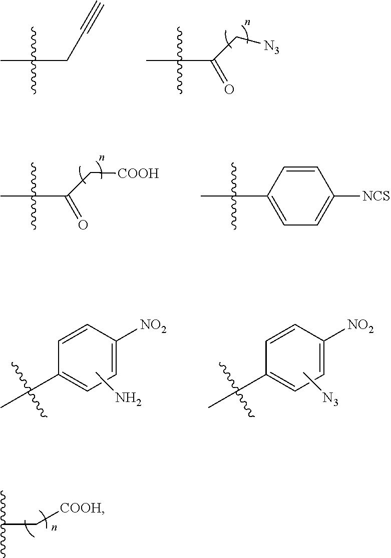
and each occurrence of ‘n’ is independently 1, 2, 3, 4, 5, 6, 7, 8, 9, 10, 11, 12, 13, 14, 15, 16, 17, 18, 19, or 20;
a polymeric molecule illustrated in FIG. 8;
a compound selected from: 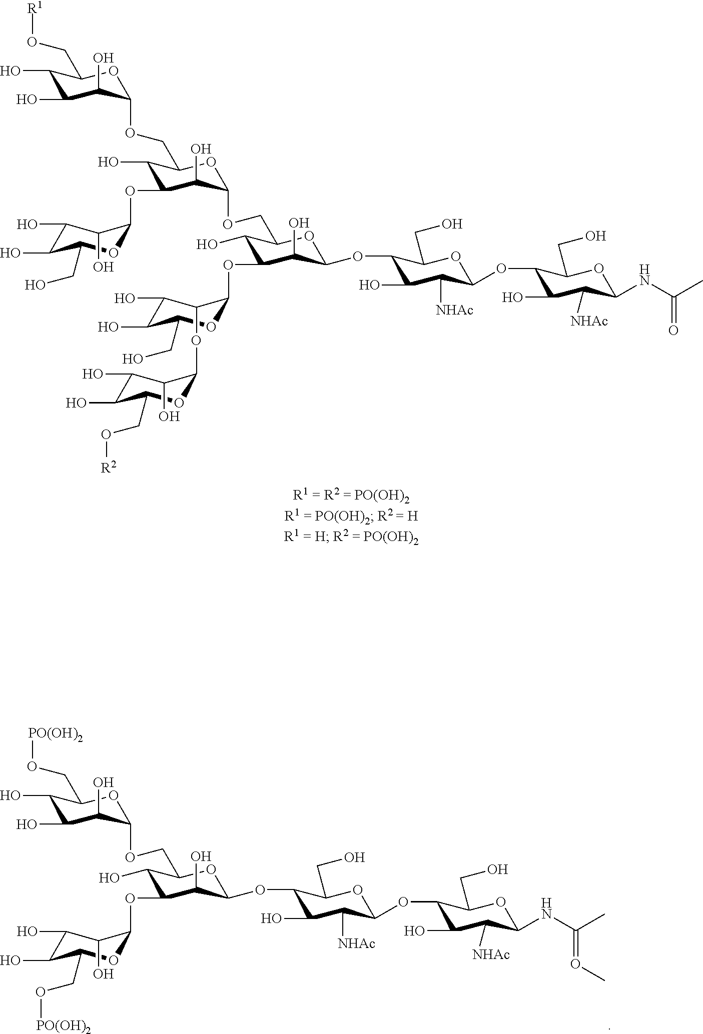
a compound selected from: 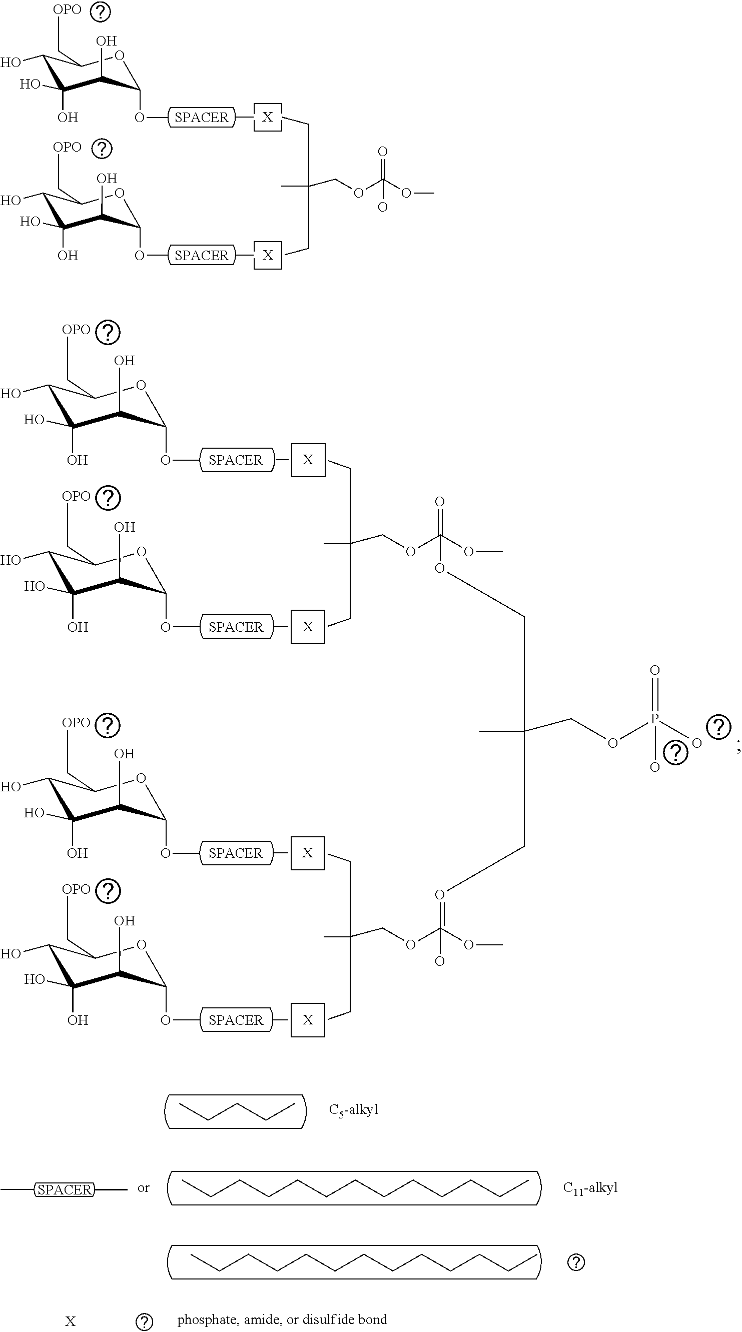
(d) a low density lipoprotein receptor-related protein 1 (LRP1) receptor binder comprising at least one amino acid sequence of SEQ ID NOs:1-9;
(e) a low density lipoprotein receptor (LDLR) binder comprising at least one amino acid sequence of SEQ ID NOs:10-35;
(f) a FcγRI receptor binder comprising at least one amino acid sequence of SEQ ID NOs:36-52;
(g) a transferrin receptor binder comprising at least one amino acid sequence of SEQ ID NOs:53-59 and 67;
(h) a macrophage scavenger receptor binder comprising at least one amino acid sequence of SEQ ID NOs:60-65;
(i) a G-protein coupled receptor binder comprising at least one of: 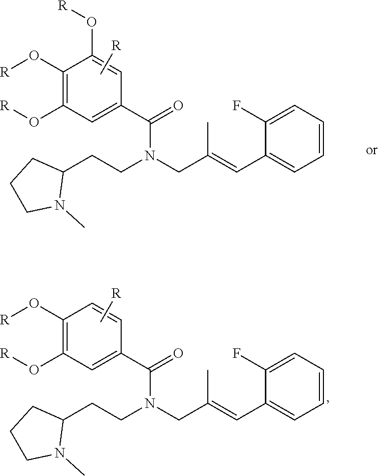
wherein each occurrence of R is independently H or C₁-C₆ alkyl;
(j) an asialoglycoprotein receptor (ASGPR) binder comprising: 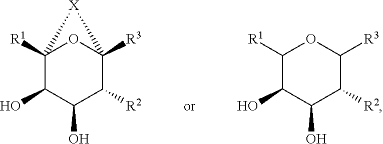
wherein:
X is a linker of 1-4 atoms in length and comprises O, S, N(R^{N1}), or C(R^{N1})(R^{N1}) groups, such that:
when X is a linker of 1 atom in length, X is O, S, N(R^{N1}), or C(R^{N1})(R^{N1}), when X is a linker of 2 atoms in length, no more than 1 atom of X is O, S, or N(R^{N1}),
when X is a linker of 3 or 4 atoms in length, no more than 2 atoms of X are independently O, S, or N(R^{N1});
wherein each occurrence of R^{N1} is independently H or C₁-C₃ alkyl optionally substituted with 1-3 independently selected halogens and/or 1-2 hydroxyl groups;
R¹ and R³ are each independently H, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}(vinyl), —O(CH₂)_{K}(vinyl), —(CH₂)_{K}(alkynyl), —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens; or
R¹ and R³ are each independently Ph(CH₂)_{K}—, which is optionally substituted with: 1-3 independently selected halogens; C₁-C₄ alkyl optionally substituted with 1-3 independently selected halogens and/or 1-2 hydroxyl groups; or C₁-C₄ alkoxy optionally substituted with 1-3 independently selected halogens and/or 1-2 hydroxyl groups; or
R¹ and R³ are each independently a group of structure:
—O—(CH₂)_{K′}—CH(OH)—(CH₂)K′—R⁷,
wherein:
R⁷ is: C₁-C₄ alkoxy optionally substituted with 1-3 independently selected halogens and/or 1-2 hydroxy groups; —NR^{N3}R^{N4}; or (CH₂)_{K′}—O—(CH₂)^{K}—CH₂—CH═CH₂;
K is 0, 1, 2, 3, or 4;
K′ is 1, 2, 3, or 4;
each occurrence of R^{N3} is independently H or C₁-C₃ alkyl optionally substituted with 1-3 independently selected halogens and/or 1-2 hydroxyl groups;
each occurrence of R^{N4} is independently H, C₁-C₃ alkyl optionally substituted with 1-3 independently selected halogens and/or 1-2 hydroxyl groups, or Ph-(CH₂)_{K}—; or
R¹ and R³ are each independently selected from the group consisting of:
—(CH₂)_{K}OH, 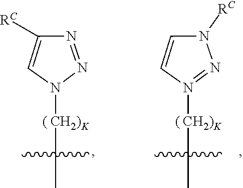
L¹—≡—, L¹—(CH₂)_{K}—, and CYC—(CH₂)_{K}—,
wherein CYC is selected from the group consisting of: 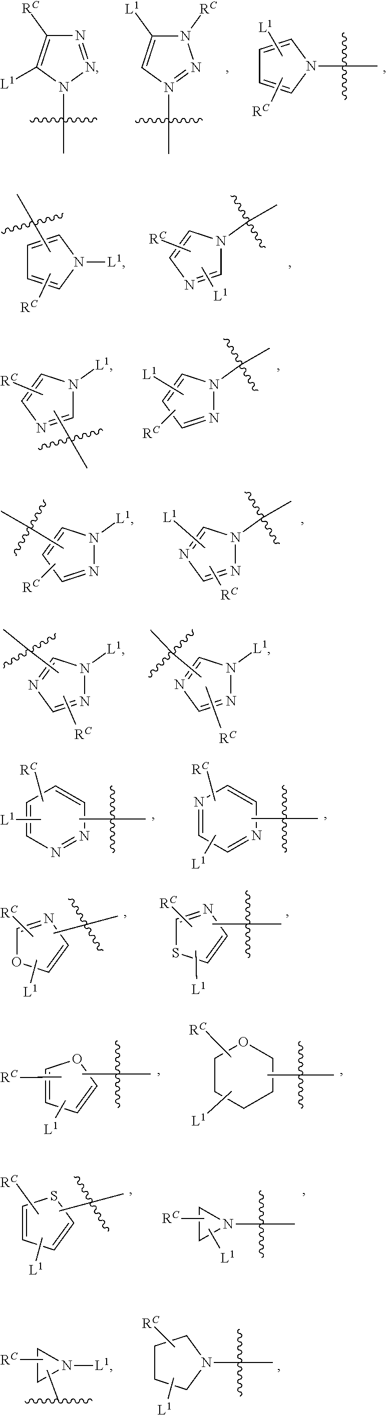 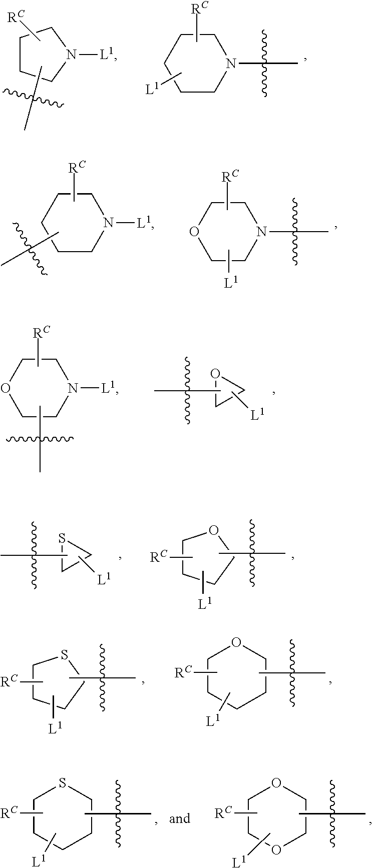
wherein:
the bond marked with [symbol] indicating the site on CYC whereto —(CH₂)_{K} is connected;
L¹ is a bond, -Linker, —CON-Linker, or —CON-Linker-CON;
R^{C} is absent, H, C₁-C₄ alkyl optionally substituted with 1-3 optionally substituted halogens and/or 1-2 hydroxyl groups, or a group of structure: 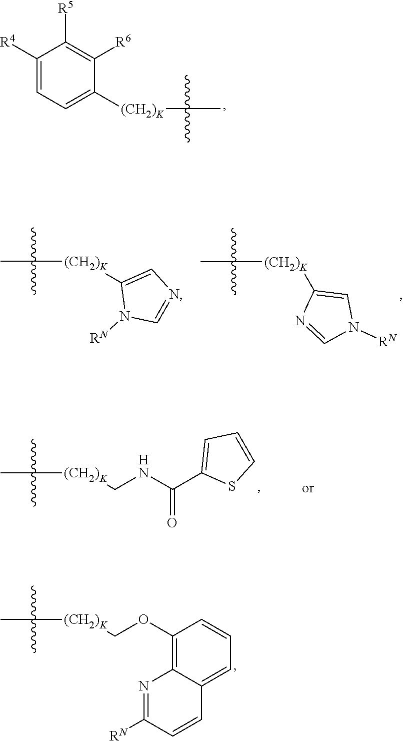
wherein:
R⁴, R⁵, and R⁶ are each independently H, F, Cl, Br, I, CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens, C₁-C₃-alkoxy optionally substituted with 1-3 independently selected halogens, —(CH₂)_{K}COOH, —(CH₂)_{K}C(═O)O—(C₁-C₄ alkyl) optionally substituted with 1-3 independently selected halogens, O—C(═O)—(C₁-C₄ alkyl) optionally substituted with 1-3 independently selected halogens, or —C(═O)—(C₁-C₄ alkyl) optionally substituted with 1-3 independently selected halogens;
each occurrence of R^{N} is independently H or C₁-C₃ alkyl optionally substituted with 1-3 independently selected halogens and/or 1-2 hydroxyl groups;
each occurrence of R^{N2} is independently H or C₁-C₃ alkyl optionally substituted with 1-3 independently selected halogens and/or 1-2 hydroxyl groups;
or
R¹ and R³ are each independently (C₃-C₈ saturated carbocyclic)—(CH₂)_{K}—, wherein the carbocyclic is further substituted with —L¹ and —R^{C};
R² is —(CH₂)_{K}—N(R^{N1})—C(═O)R^{AM}, wherein:
R^{AM} is H, C₁-C₄ alkyl optionally substituted with 1-3 independently selected halogens and/or 1-2 hydroxyl groups, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, —C(═O)(C₁-C₄ alkyl) optionally substituted with 1-3 independently selected halogens, or —(CH₂)_{K}—NR^{N3}R^{N4}; or
R² is 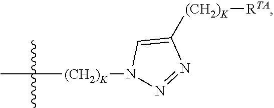
wherein:
R^{TA} is H, CN, NR^{N1}R^{N2}, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens, or
R^{TA} is C₃-C₁₀ aryl or a 3- to 10-membered heteroaryl group containing 1-5 non-carbon ring atoms, each of the aryl or heteroaryl groups being optionally substituted with 1-3 groups independently selected from CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens and/or 1-2 hydroxyl groups, —(C₁-C₃-alkoxy) optionally substituted from 1-3 independently selected halogens, —(CH₂)_{K}COOH, —(CH₂)_{K}C(═O)O—(C₁-C₄ alkyl) optionally substituted with 1-3 independently selected halogens, —OC(═O)(C₁-C₄ alkyl) optionally substituted with 1-3 independently selected halogens, or —(CH₂)_{K}C(═O)—(C₁-C₄ alkyl) optionally substituted with 1-3 independently selected halogens, or
R^{TA} is 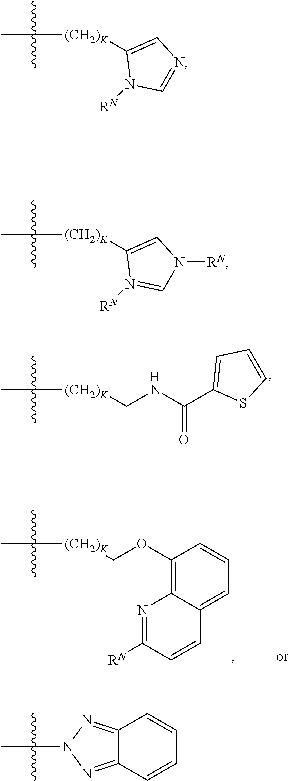
optionally substituted with 1-3 C₁-C₃ alkyl groups optionally substituted with 1-3 independently selected halogens, or R^{TA} is 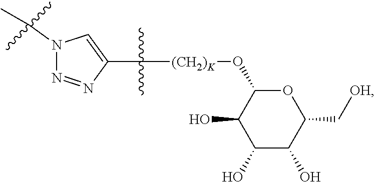
wherein each —(CH₂)_{K} group is optionally substituted with 1-4 C₁-C₃ alkyl groups optionally substituted with 1-3 fluoro groups or 1-2 hydroxyl groups.

15. The compound of claim 14, wherein:
the X in ASGPRBM is —O—C(R^{N1})(R^{N1})—, —C(R^{N1})(R^{N1})—O—, —S—C(R^{N1})(R^{N1})—, —C(R^{N1})(R^{N1})—S—, —N(R^{N1})—C(R^{N1})(R^{N1})—, —C(R^{N1})(R^{N1})—N(R^{N1})—, or —C(R^{N1})(R^{N1})—C(R^{N1})(R^{N1})—, when X is 2 atoms in length;
the X in ASGPRBM is —O—C(R^{N1})(R^{N1})—C(R^{N1})(R^{N1})—, —C(R^{N1})(R^{N1})—O—C(R^{N1})(R^{N1})—, —O—C(R^{N1})(R^{N1})—O—, —O—C(R^{N1})(R^{N1})—S—, —O—C(R^{N1})(R^{N1})—N(R^{N1})—, —S—C(R^{N1})(R^{N1})—C(R^{N1})(R^{N1})—, —C(R^{N1})(R^{N1})—S—C(R^{N1})(R^{N1})—, —C(R^{N1})(R^{N1})—C(R^{N1})(R^{N1})—S, —S—C(R^{N1})(R^{N1})—S—, —S—C(R^{N1})(R^{N1})—O—, —S—C(R^{N1})(R^{N1})—N(R^{N1})—, —N(R^{N1})—C(R^{N1})(R^{N1})—C(R^{N1})(R^{N1})—, —C(R^{N1})(R^{N1})—N(R^{N1})—C(R^{N1})(R^{N1})—, —C(R^{N1})(R^{N1})—C(R^{N1})(R^{N1})—N(R^{N1})—, —N(R^{N1})—C(R^{N1})(R^{N1})—N(R^{N1})—, or —C(R^{N1})(R^{N1})—C(R^{N1})(R^{N1})— C(R^{N1})(R^{N1}), when X is 3 atoms in length; or
the X in ASGPRBM is —O—C(R^{N1})(R^{N1})—C(R^{N1})(R^{N1})—C(R^{N1})(R^{N1})—, —C(R^{N1})(R^{N1})—O—C(R^{N1})(R^{N1})—C(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})—, —C(R^{N1})(R^{N1})—S—C(R^{N1})(R^{N1})—C(R^{N1})(R^{N1})—, —C(R^{N1})(R^{N1})—C(R^{N1})(R^{N1})—S—C(R^{N1})(R^{N1})—, —S—C(R^{N1})(R^{N1})—S—C(R^{N1})(R^{N1})—, —N(R^{N1})—C(R^{N1})(R^{N1})—C(R^{N1})(R^{N1})— C(R^{N1})(R^{N1})—, or —C(R^{N1})(R^{N1})—N(R^{N1})—C(R^{N1})(R^{N1})—C(R^{N1})(R^{N1})—, when X is 4 atoms in length.

16. The compound of claim 14, wherein X is OCH₂ and R^{N1} is H, or wherein X is CH₂O and R^{N1} is H.

17. The compound of claim 14, wherein the ASGPRBM comprises the structure:

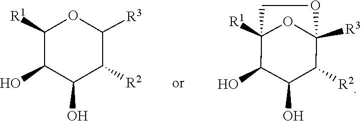

18. The compound of claim 14, wherein the ASGPRBM group comprises: 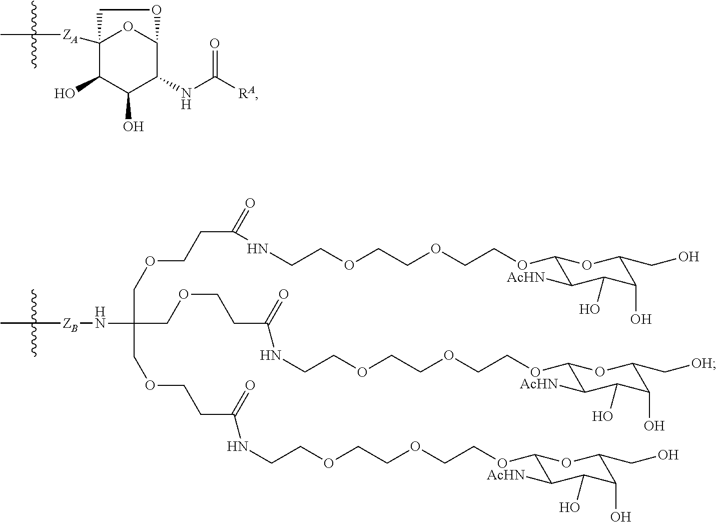
wherein:
R^{A} is C₁-C₃ alkyl optionally substituted with 1-5 independently selected halogens;
Z_{A} is —(CH₂)_{IM}—, —O—(CH₂)_{IM}—, —S—(CH₂)_{IM}—, —NR^{M}—(CH₂)_{IM}—, —C(═O)—(CH₂)_{IM}—, a PEG group containing from 1 to 8 ethylene glycol residues, or —C(O)(CH₂)_{IM}NR^{M}—;
Z_{B} is absent, —(CH₂)_{IM}—, —C(═O)—(CH₂)_{IM}—, or —C(═O)(CH₂)_{IM}—NR^{M}—;
R^{M} is H or C₁-C₃ alkyl optionally substituted with 1-2 hydroxyl groups; and
each occurrence of IM is independently 0, 1, 2, 3, 4, 5, or 6;
3 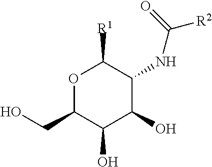
4

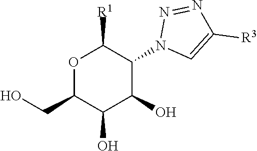

| | R¹ | # | R² | # | R² | # | R³ | # | R³ | # | R³ |
|---|---|---|---|---|---|---|---|---|---|---|---|
| a | Me | 1 | Me | 9 | CH₂NH₂ | 1 | Ph | 10 | 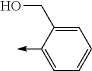 | 14 | 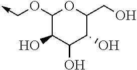 |
| b | 4- OMePh | 2 | [symbol] | 10 | CH₂CF₃ | 2 | 4-OMePh | 11 | 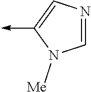 | 15 | CH₂OH |
| | | 3 | i-Pr | 11 | 2-furyl | 3 | 3-OMePh | 12 | 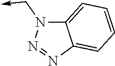 | 16 | CH₂NH₂ |
| | | 4 | t-Bu | 12 | Ph | 4 | 3-NH₂Ph | 13 | 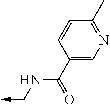 | 17 | CH₂NHMe |
| | | 5 | CCl₂ | 13 | 4-OMePh | 5 | 4-NMe₂Ph | | | 18 | CH₂NMe₂ |
| | | 6 | CF₃ | 14 | 3-OMePh | 6 | 2-pyridyl | | | 19 | CO₂H |
| | | 7 | Ot-Bu | 15 | 4-CNPh | 7 | 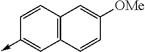 | | | 20 | CH₂NHCOPh |
| | | 8 | CH₂CO₂H | 16 | 3-pyridyl | 8 | 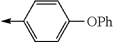 | | | 21 | CH₂NHCOMe |
| | | | | 17 | 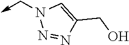 | 9 | 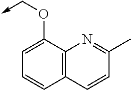 | | | | |
| | | R¹ | | R¹ |
|---|---|---|---|---|
| 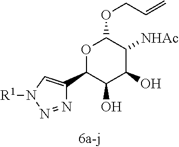 | a | Me | g | 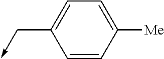 |
| | b | 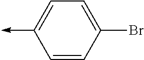 | h | 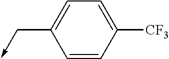 |
| | c | 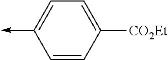 | i | 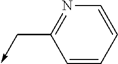 |
| | d | 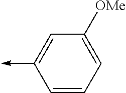 | j | 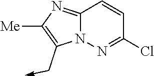 |
| | e | 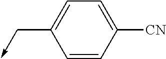 | | |
| | f | 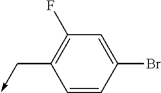 | | |
| | | R² | | R² |
| 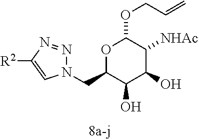 | a | CH₂OH | g | 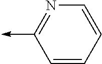 |
| | b | 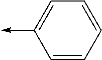 | h | 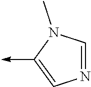 |
| | c | 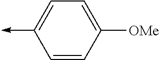 | i | 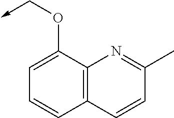 |
| | d | 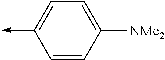 | j | 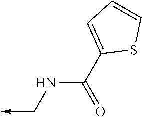 |
| | e | 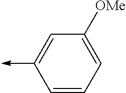 | | |
| | f | 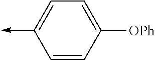 | | |

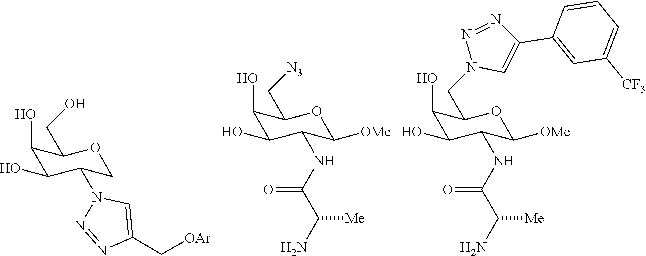

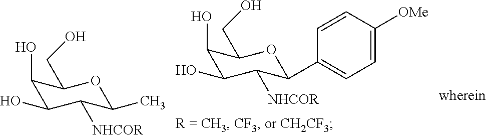

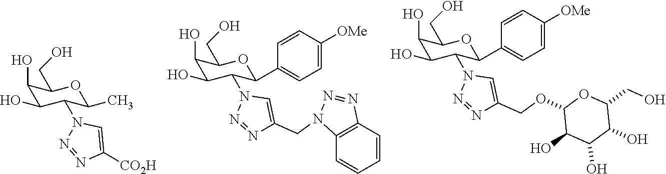

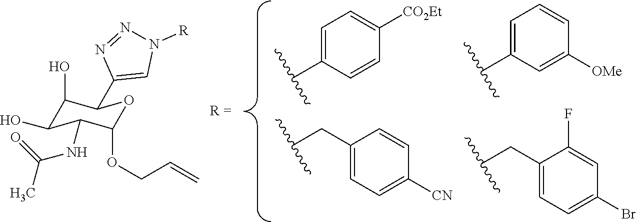

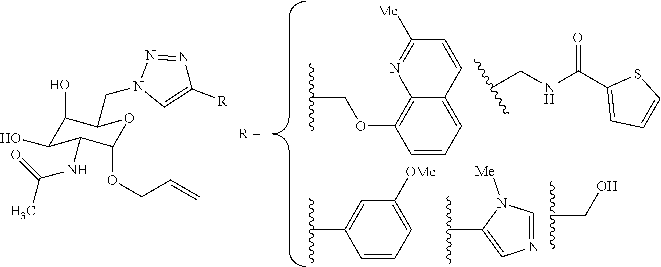

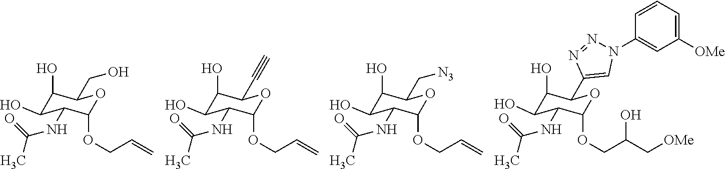

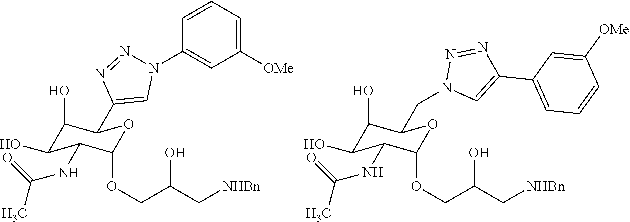

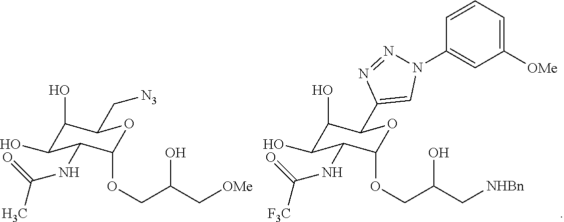

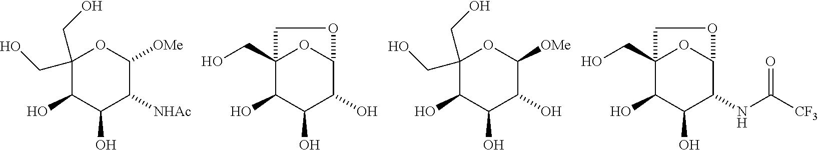 [SMILES: CO[C@H]1OC(CO)(CO)[C@H](O)[C@H](O)[C@H]1NC(C)=O ; OC[C@@]12CO[C@@H](O1)[C@H](O)[C@@H](O)[C@H]2O ; CO[C@@H]1OC(CO)(CO)[C@H](O)[C@H](O)[C@H]1O ; O=C(N[C@H]1[C@H]2OC[C@](CO)(O2)[C@H](O)[C@@H]1O)C(F)(F)F]
| | | R |
|---|---|---|
| 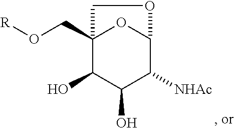 | 16a 16b 16c 16d 16e 16f | Et n-C₃H₇ n-C₄H₉ n-C₅H₁₁ n-C₆H₁₃ (CH₂CH₂O)₄Me |

[symbol]

indicates data missing or illegible when filed
or

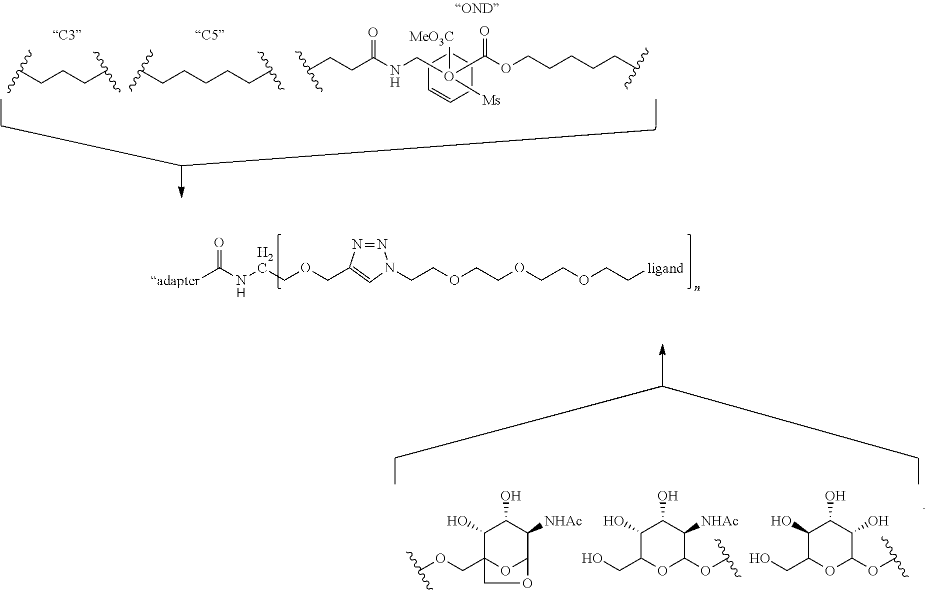

19. The compound of claim 1, wherein the Linker is a polyethylene glycol containing linker having 1-12 ethylene glycol residues.

20. The compound of claim 1,
wherein the Linker comprises the structure:
—CH₂CH₂(OCH₂CH₂)ₘOCH₂—, —(CH₂)ₘCH₂—, —[N(Rₐ)—CH(R^{b})(C═O)]ₘ—,
or a polypropylene glycol or polypropylene-co-polyethylene glycol group containing 1-100 alkylene glycol units;
wherein each R^{a} is independently H, C₁-C₃ alkyl, or C₁-C₆ alkanol, or
combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
wherein m is an integer ranging from 1 to 15;
or
wherein the Linker comprises the structure
—[N(R′—(CH₂)₁₋₁₅—C(═O)]—,
wherein R′ is H or a C₁-C₃ alkyl optionally substituted with 1-2 hydroxyl groups, and
m is an integer ranging from 1 to 100; or
wherein the Linker comprises the structure:
-Z-D-Z′—,
wherein:
Z and Z′ are each independently a bond, —(CH₂)ᵢ—O—, —(CH₂)ᵢ—S—, —(CH₂)ᵢ—N(R)—, 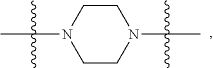 [SMILES: *N1CCN(*)CC1]
—(CH₂)ᵢ—C(R²)═C(R²)— (cis or trans), —(CH₂)ᵢ—≡—, or —Y—C(═O)—Y—;
each R is independently H, C₁-C₃ alkyl, or C₁-C₆ alkanol;
each R² is independently H or C₁-C₃ alkyl;
each Y is independently a bond, O, S, or N(R);
each i is independently 0 to 100;
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X¹ is O, S, or N(R);
j is an integer ranging from 1 to 100;
m′ is an integer ranging from 1 to 100;
n is an integer ranging from 1 to 100; or
wherein the Linker comprises the structure:
—CH₂—(OCH₂CH₂)ₙ—CH₂—, —(CH₂CH₂O)_{n′}CH₂CH₂—, or —(CH₂CH₂CH₂₀)ₙ—,
wherein each n and n′ is independently an integer ranging from 1 to 25; or
wherein the Linker comprises a structure:
-PEG-CON-PEG-
wherein each PEG is independently a polyethylene glycol group containing from 1-12 ethylene glycol residues and CON is a triazole group

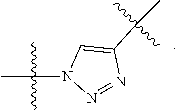 [SMILES: *c1cn(*)nn1]

21. The compound of claim 1,
wherein the CON comprises a structure: 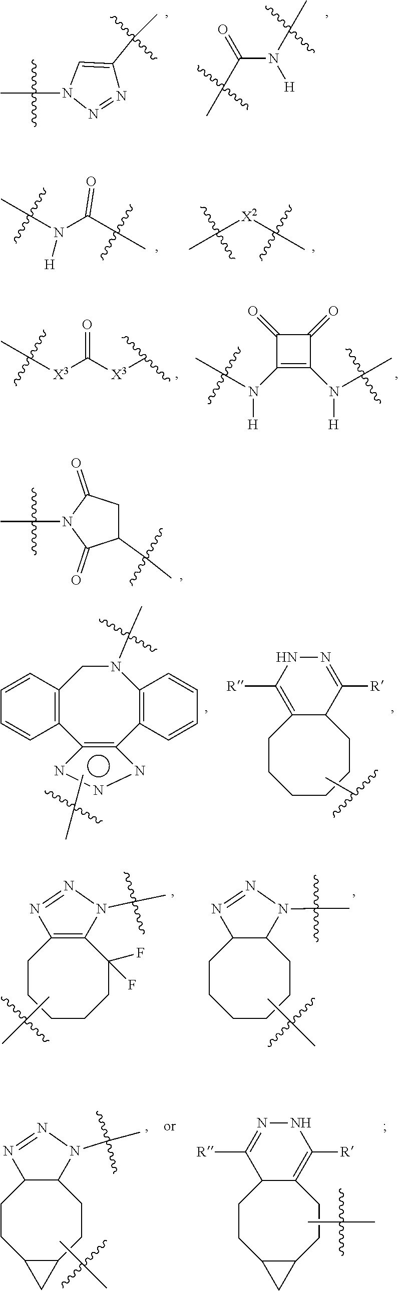
wherein R′ and R″ are each independently H, methyl, or a bond; or
wherein the CON comprises a structure:
—C(═O)—N(R¹)—(CH₂)_{n″}—N(R¹)C(═O)—,
—N(R¹)—C(═O)(CH₂)_{n″}—C(═O)N(R¹)—, or
—N(R¹)—C(═O)(CH₂)_{n″}—N(R¹)C(═O)—;
wherein each R¹ is independently H or C₁-C₃ alkyl, and n″ is independently an integer from 0 to 8, in certain embodiments 1 to 7, in certain embodiments 1, 2, 3, 4, 5 or 6;
or
wherein the CON comprises a structure: 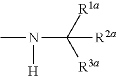
wherein:
R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}—, or —(CH₂)_{M2}O—(CH₂)_{M1}—C(O)NR⁴—, with the proviso that R^{1a}, R^{2a} and R^{3a} are not simultaneously H;
each M1 is independently 1, 2, 3, or 4;
each M2 is independently 0, 1, 2, 3, or 4;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same Ria, R^{2a} and Ria cannot all be simultaneously 0; or
wherein the CON comprises a structure:

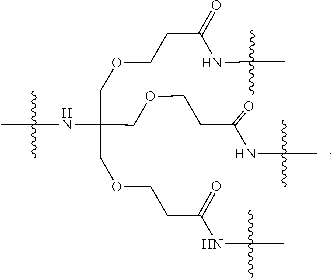 [SMILES: *NC(=O)CCOCC(COCCC(=O)N*)(COCCC(=O)N*)N*]

22. The compound of claim 1, wherein the extracellular protein comprises 1-4043-amyloid, 5′-nucleotidase, activated F9, F10, activin receptor-like kinase 1, alpha-fetoprotein, amyloid, angiopoietin 2, angiopoietin 3, anthrax toxin, AOC3, AOC3 (VAP-1), Bacillus anthracis anthrax, BAFF, beta amyloid, c-Met, C1s, C242 antigen, C5, CA-125, calcitonin, calcitonin gene-related peptide, calcitonin gene-related peptide alpha, Canis lupus familiaris IL31, carbonic anhydrase 9 (CA-IX), CEA, CEA-related antigen, CEACAM5, CFD, CGRP, clumping factor A, coagulation factor III, complement C5a, CSF1, MCSF, CSF2, dabigatran, E. coli shiga toxin type-1, E. coli shiga toxin type-2, EGFL7, endotoxin, episialin, FGF 23, fibrin II, beta chain, fibronectin extra domain-B, folate hydrolase, GDF-8, gelatinase B, GMCSF, growth differentiation factor 8, hemagglutinin, hemagglutinin HA, HGF, HIV-1, HNGF, Hsp90, human beta-amyloid, human scatter factor receptor kinase, human TNF, IFN-α, IFN-γ, IgE, IgE Fc region, IGF1, IGF2, IGHE, IL 17A, IL 17A and IL 17F, IL 20, IL-1, IL-12, IL-23, IL-13, IL-17, IL-1(3, IL-22, IL-4, IL-5, IL-6, IL17A and IL17F, IL1A, IL2, IL23, IL23A, IL31RA, IL6, IL6R, IL9, ILGF2, Influenza A hemagglutinin, influenza A virus hemagglutinin, influenza A virus hemagglutinin HA, interferon gamma, interferon gamma-induced protein, interleukin 1 alpha, interleukin 13, interleukin 17 alpha, interleukin 17 alpha, TNF, interleukin 17A, kallikrein, LOXL2, LRRC15, LTA, MASP-2, MCP-1, MIF, MST1R (aka RON), MUC1, myostatin, NACP, NCA-90 (granulocyte antigen), neural apoptosis-regulated proteinase 1, NGF, NOGO-A, Notch 1, NRP1, oxLDL, PCSK9, PD-L1, phosphatidylserine, RANKL, RGMA, root plate-specific spondin 3, RTN4, sclerostin, SDC1, serum amyloid A protein, serum amyloid P component, SOST, Staphylococcus aureus alpha toxin, tau protein, TFPI, TGF beta 1, TGF beta 2, TGF-β, TNF-α, TROP-2, TSLP, VEGF-A, VEGF-A and Ang-2, VEGFA, or VWF.

23. The compound of claim 1, wherein the Ab is a monoclonal antibody.

24. The compound of claim 1, wherein the antibody comprises: Abagovomab, Abrezekimab, Adalimumab, Aducanumab, Afasevikumab, Afelimomab, Alirocumab, Altumomab, Altumomab pentetate, Andecaliximab, Anrukinzumab, Arcitumomab, Ascrinvacumab, Atezolizumab, Atidortoxumab, Atinumab, Avelumab, Bapineuzumab, Bavituximab, Belimumab, Bermekimab, Besilesomab, Bevacizumab, Biciromab, Bimekizumab, Birtamimab, Blosozumab, Bococizumab, Brazikumab, Briakinumab, Brodalumab, Brolucizumab, Brontictuzumab, Burosumab, Cabiralizumab, Canakinumab, Cantuzumab, Cantuzumab ravtansine, Caplacizumab, Carlumab, Cergutuzumab, Cergutuzumab amunaleukin, Certolizumab, Certolizumab pegol, Cibisatamab, Clazakizumab, Clivatuzumab, Clivatuzumab tetraxetan, Concizumab, Crenezumab, Dectrekumab, Denosumab, Dezamizumab, Diridavumab, Domagrozumab, Dorlimomab, Dorlimomab aritox, Durvalumab, Dusigitumab, Eculizumab, Edobacomab, Efungumab, Eldelumab, Elezanumab, Elsilimomab, Emactuzumab, Emapalumab, Emicizumab, Enokizumab, Epitumomab, Epitumomab cituxetan, Eptinezumab, Erenumab, Evinacumab, Evolocumab, Faricimab, Fasinumab, Fezakinumab, Ficlatuzumab, Firivumab, Fletikumab, Fontolizumab, Fremanezumab, Fresolimumab, Frovocimab, Frunevetmab, Fulranumab, Galcanezumab, Gantenerumab, Gatipotuzumab, Gedivumab, Gevokizumab, Gimsilumab, Girentuximab, Golimumab, Gosuranemab, Guselkumab, Idarucizumab, Igovomab, Imalumab, Indatuximab, Indatuximab ravtansine, Infliximab, Istiratumab, Ixekizumab, Labetuzumab, Lacnotuzumab, Lampalizumab, Lanadelumab, Landogrozumab, Lebrikizumab, Lemalesomab, Lendalizumab, Lenzilumab, Lerdelimumab, Lesofavumab, Ligelizumab, Lodelcizumab, Lokivetmab, Lutikizumab, Marstacimab, Mepolizumab, Metelimumab, Mirikizumab, Nacolomab, Nacolomab tafenatox, Namilumab, Narnatumab, Navivumab, Naxitamab, Nebacumab, Nemolizumab, NEOD, Nerelimomab, Nesvacumab, Netakimab, Nofetumomab, Nofetumomab merpentan, Obiltoxaximab, Oleclumab, Olendalizumab, Olokizumab, Omalizumab, OMS, Onartuzumab, Oregovomab, Orticumab, Otilimab, Ozanezumab, Ozoralizumab, Parsatuzumab, Pascolizumab, Pasotuxizumab, Pateclizumab, Pemtumomab, Perakizumab, Pexelizumab, Placulumab, Ponezumab, Prasinezumab, Pritoxaximab, Quilizumab, Radretumab, Ralpancizumab, Ranevetmab, Ranibizumab, Ravulizumab, Raxibacumab, REGN-EB, Remtolumab, Reslizumab, Rilotumumab, Risankizumab, Romilkimab, Romosozumab, Rontalizumab, Rosmantuzumab, Sacituzumab, Sacituzumab govitecan, Samrotamab, Samrotamab vedotin, Sarilumab, Secukinumab, Setoxaximab, Setrusumab, Sifalimumab, Siltuximab, Simtuzumab, Sirukumab, Sofituzumab, Sofituzumab, Sofituzumab vedotin, Solanezumab, Sontuzumab, Stamulumab, Sulesomab, Sutimlimab, Suvizumab, Suvratoxumab, Tabalumab, Tacatuzumab, Tacatuzumab tetraxetan, Talizumab, Tanezumab, Tefibazumab, Telimomab, Telimomab aritox, Tesidolumab, Tezepelumab, Tibulizumab, Tildrakizumab, Timolumab, Tisotumab, Tisotumab vedotin, Tralokinumab, Trevogrumab, Urtoxazumab, Ustekinumab, Vanucizumab, Vapaliximab, Varisacumab, Vepalimomab, Vesencumab, Vobarilizumab, Vunakizumab, or Xentuzumab.

25. A pharmaceutical composition comprising at least one pharmaceutically acceptable excipient and at least one compound of claim 1.

26. The pharmaceutical composition of claim 25, further comprising another therapeutically active compound.

27. A compound selected from the group consisting of 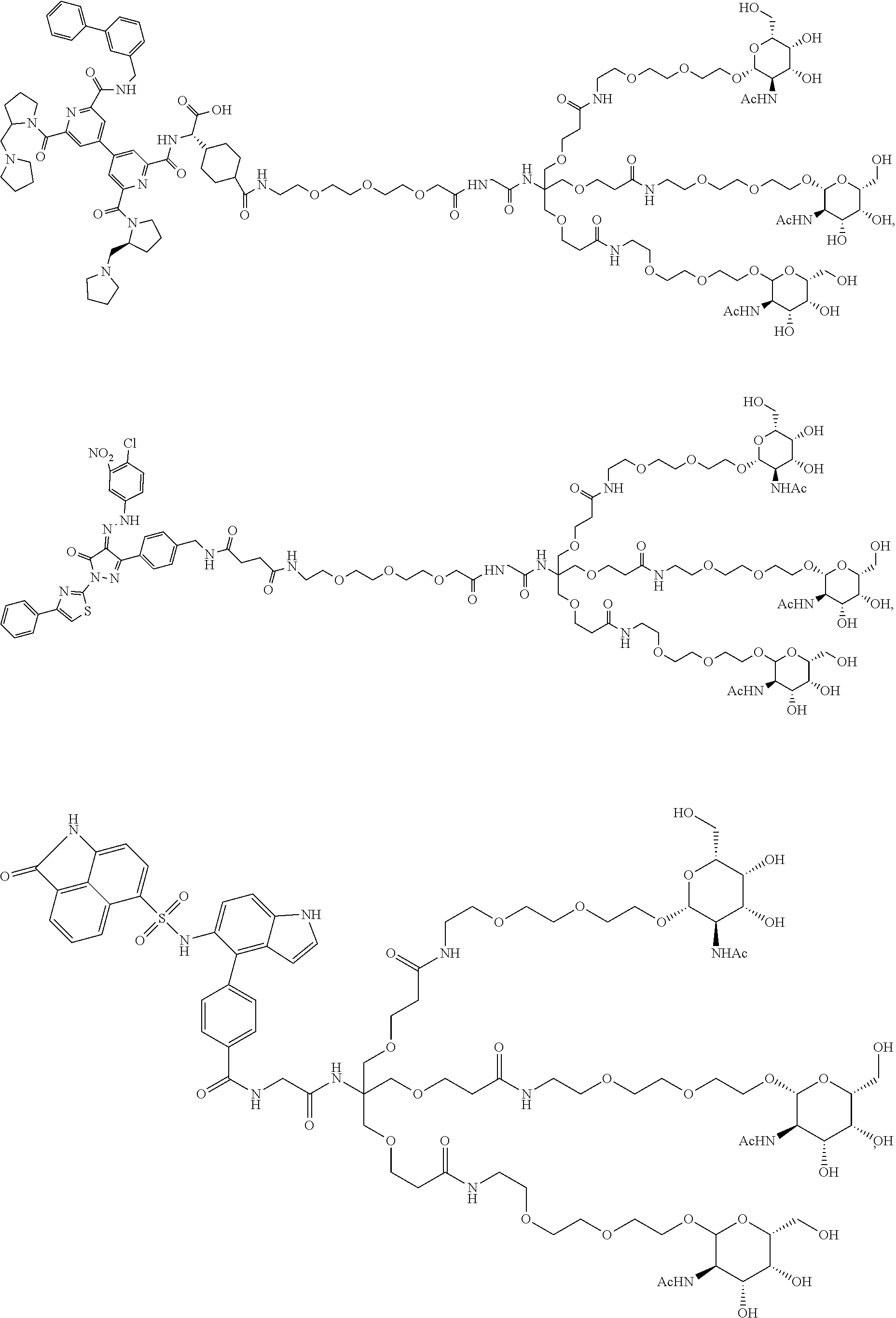 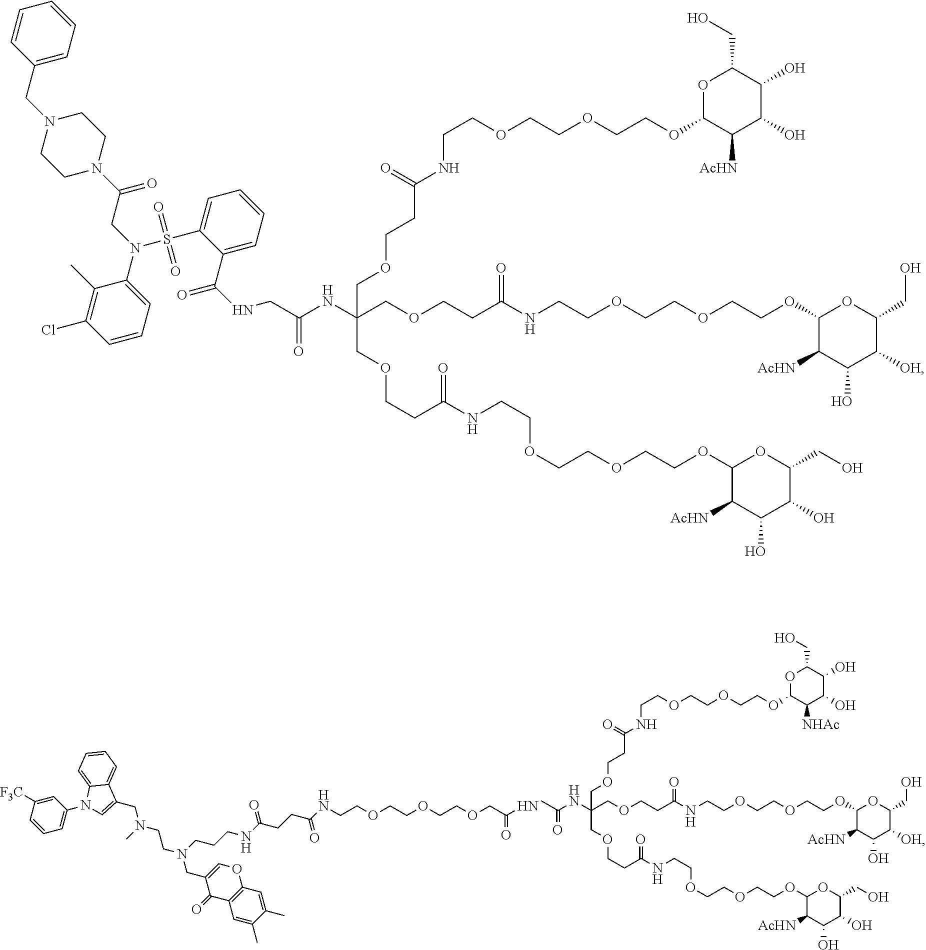 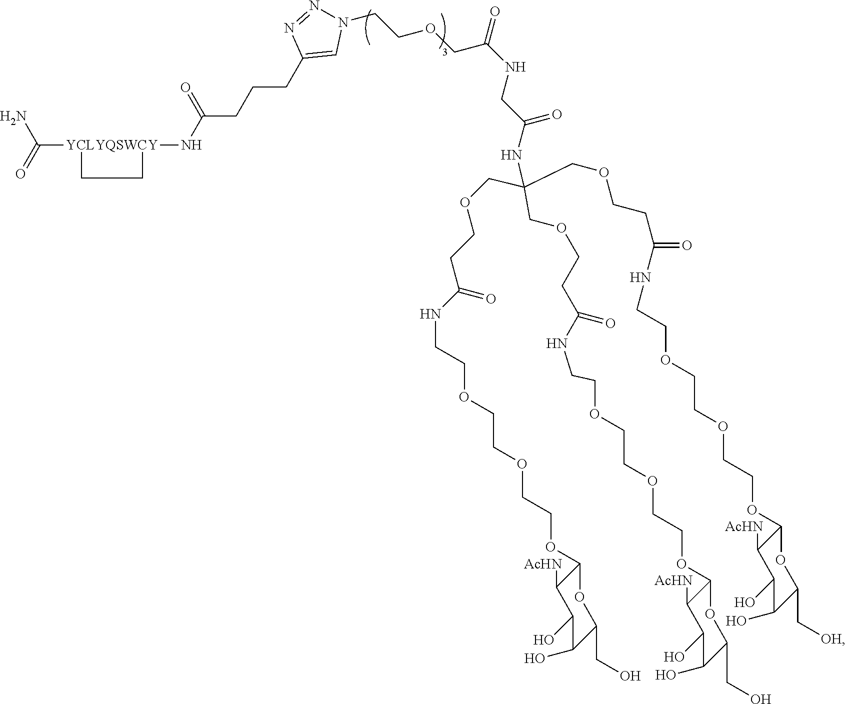 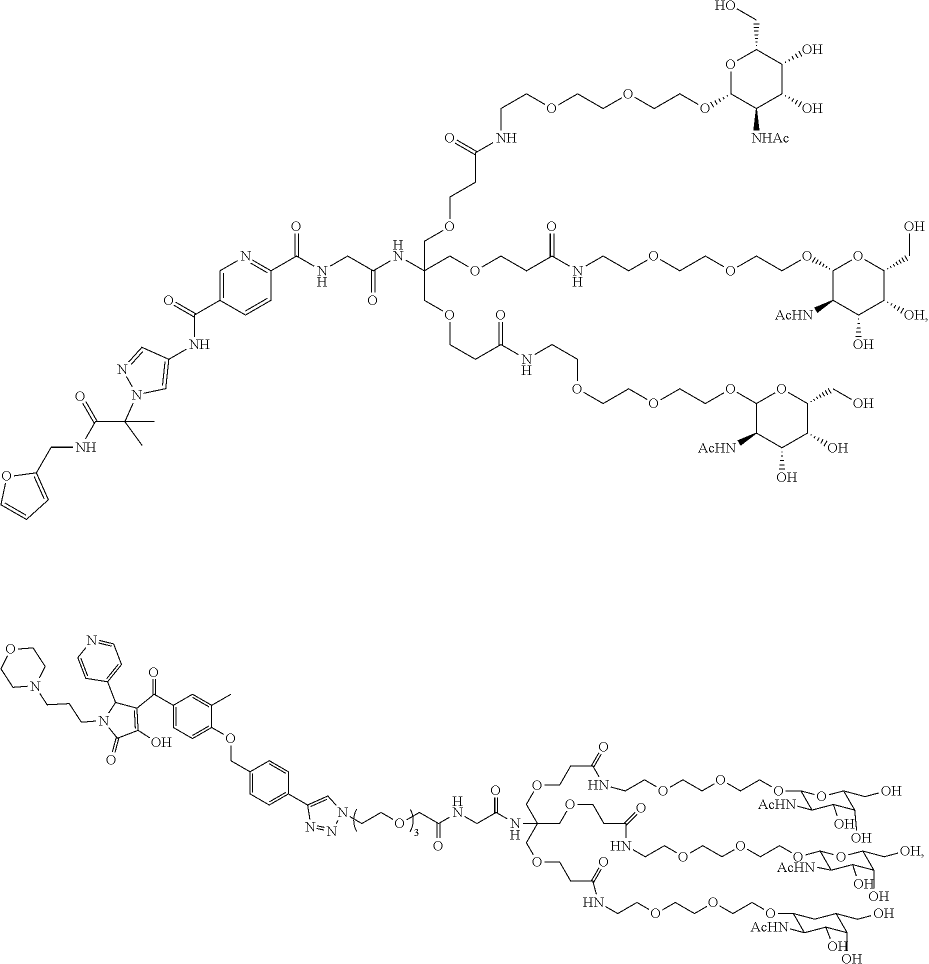 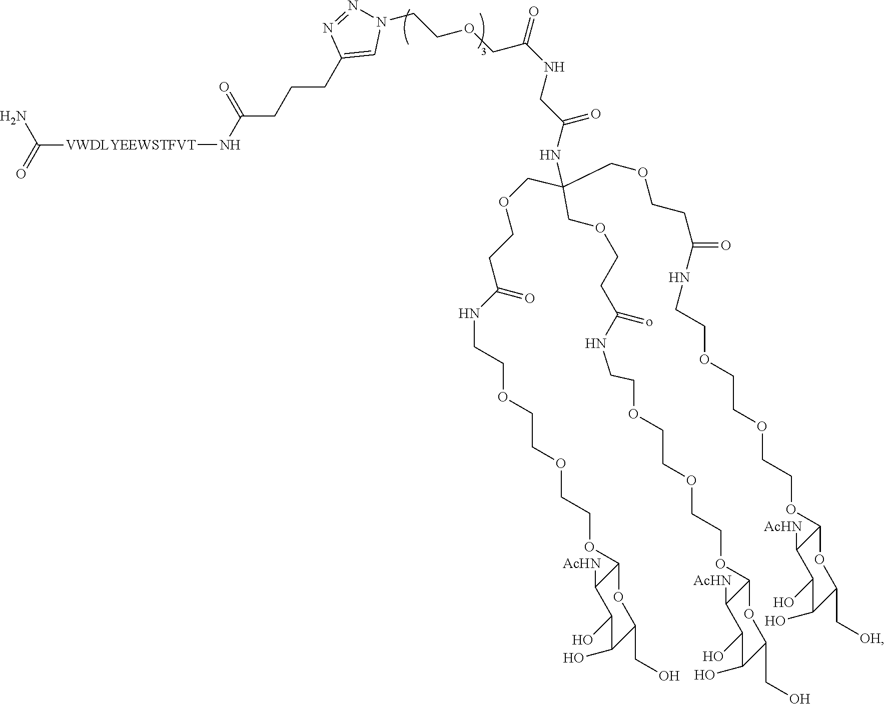 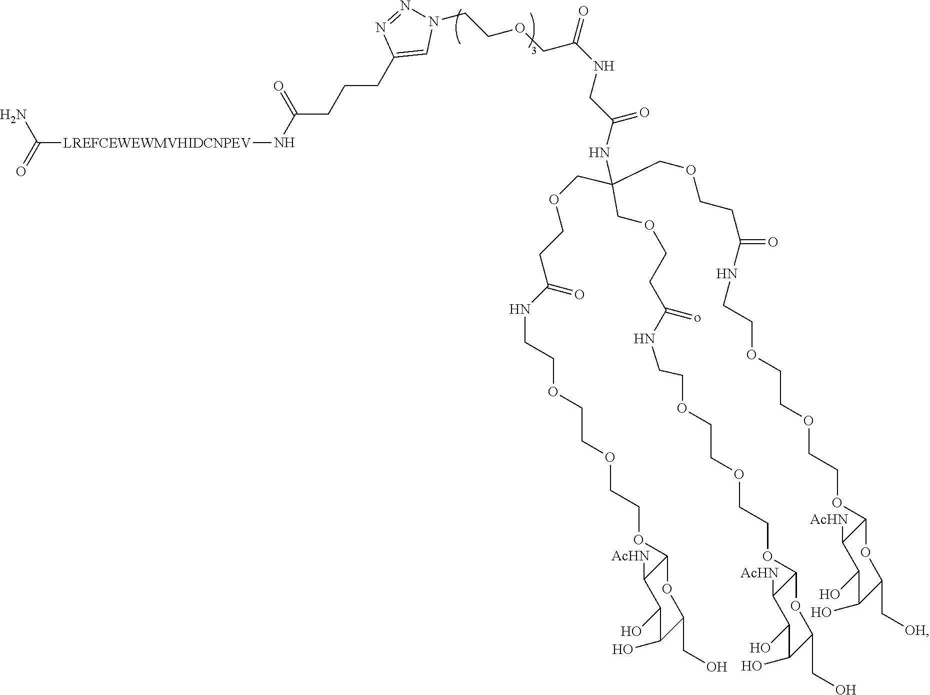 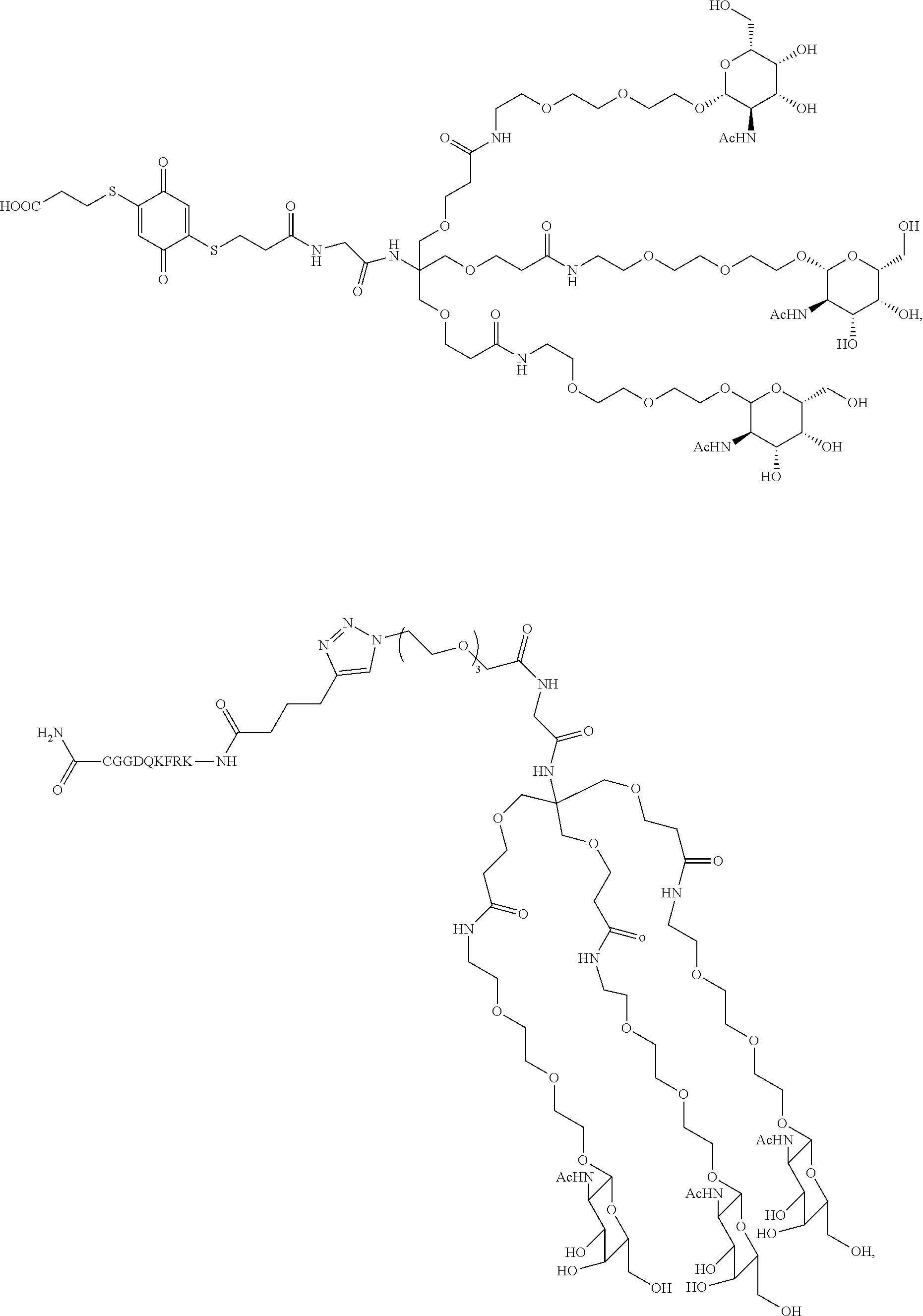 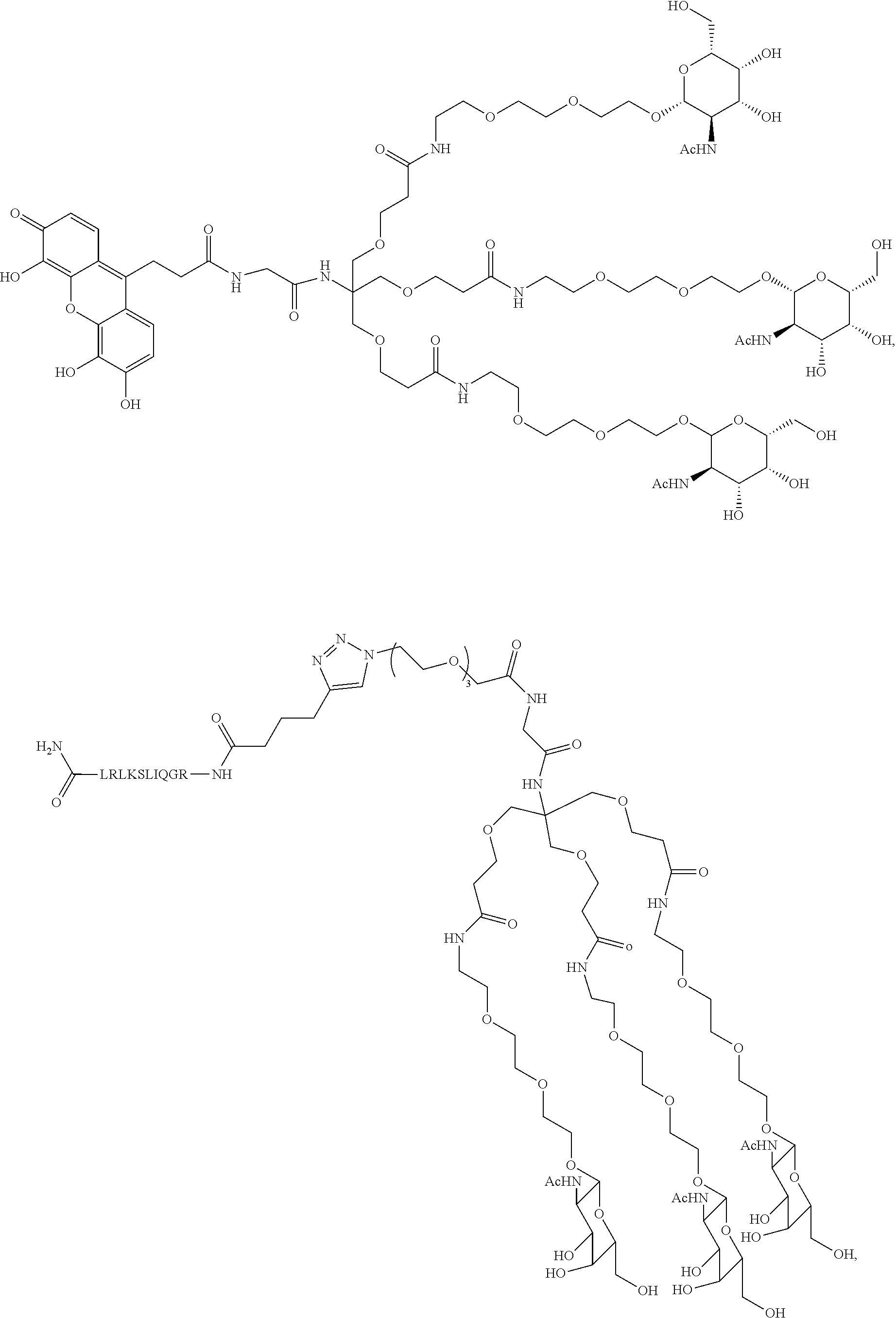 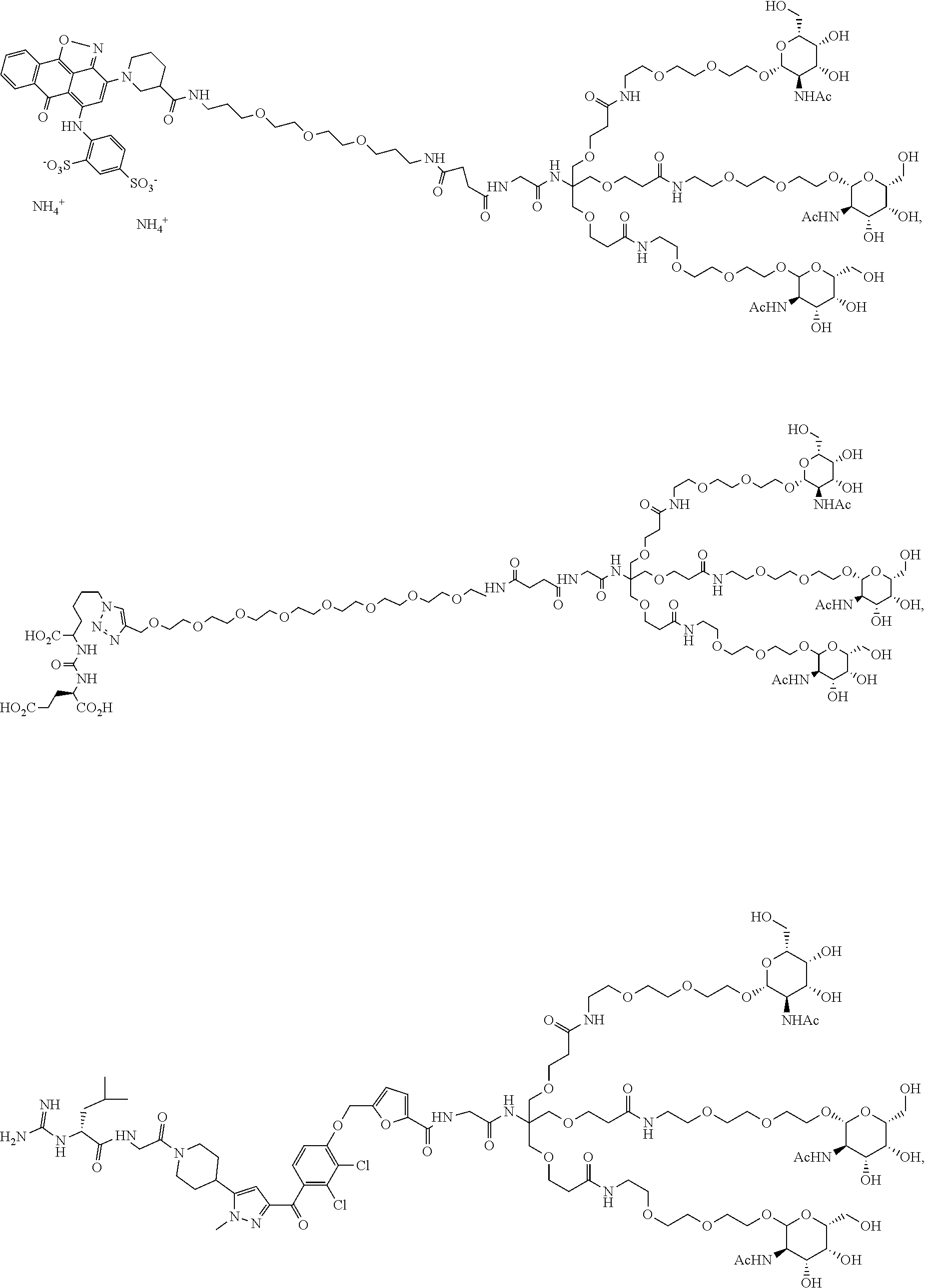 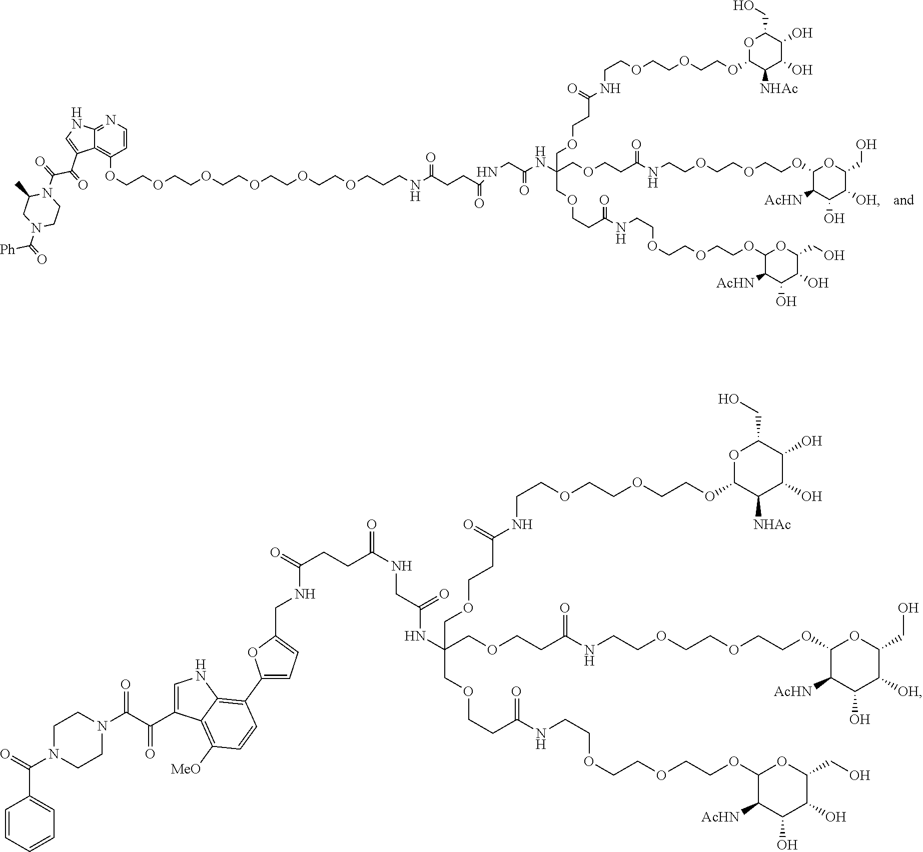
a salt, stereoisomer, or solvate thereof.

28. The compound of claim 1, having one of the following formulae: 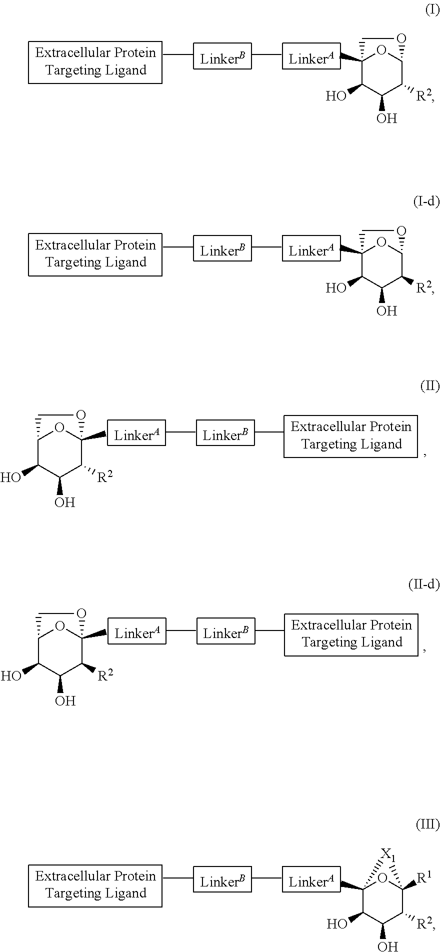 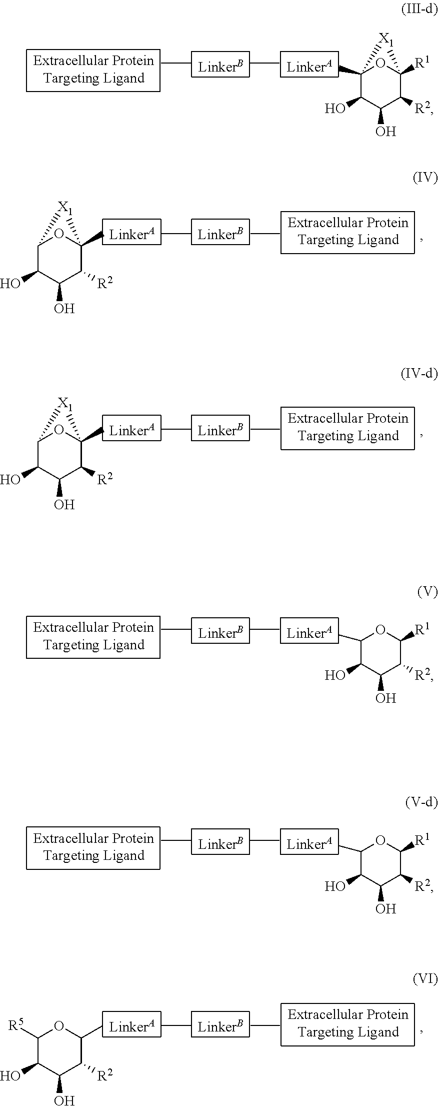 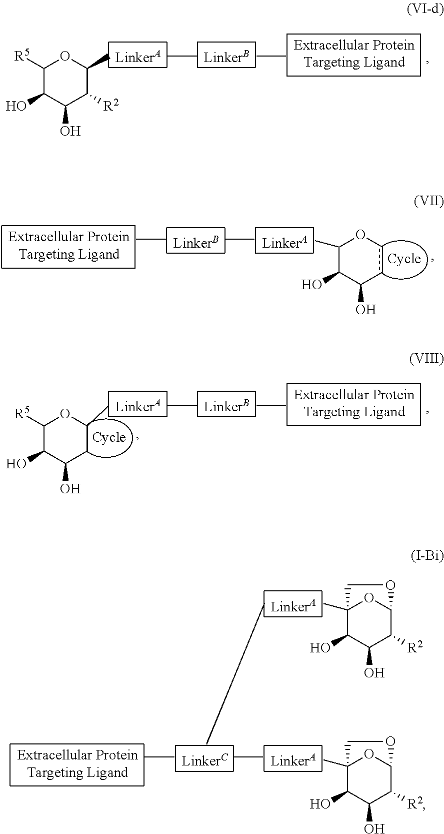 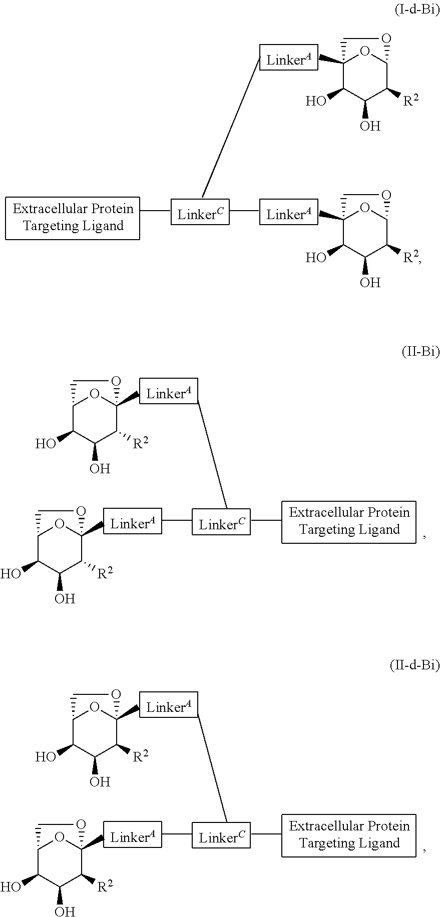 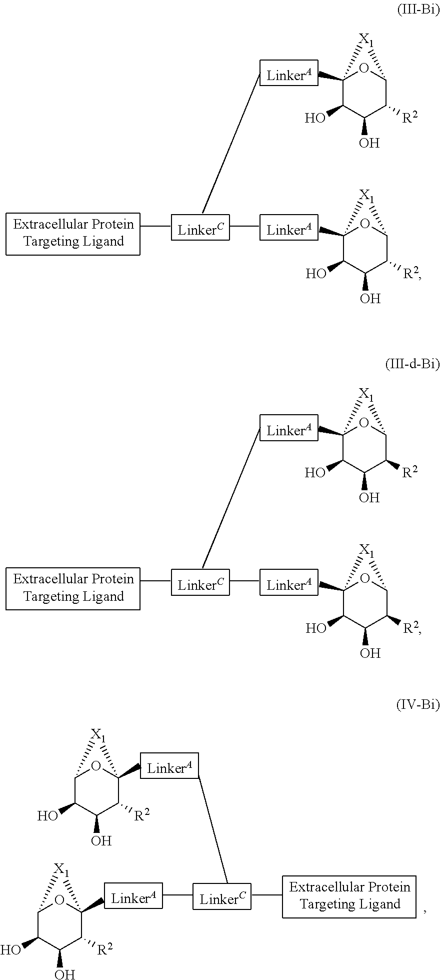 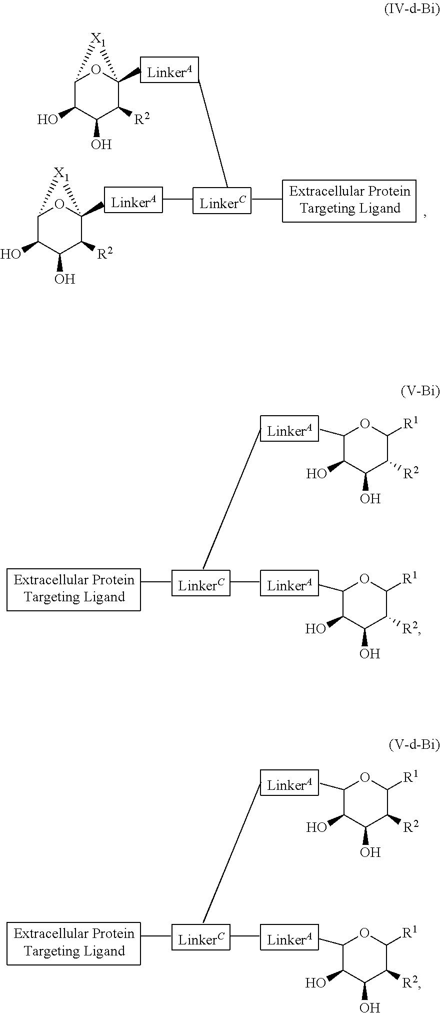 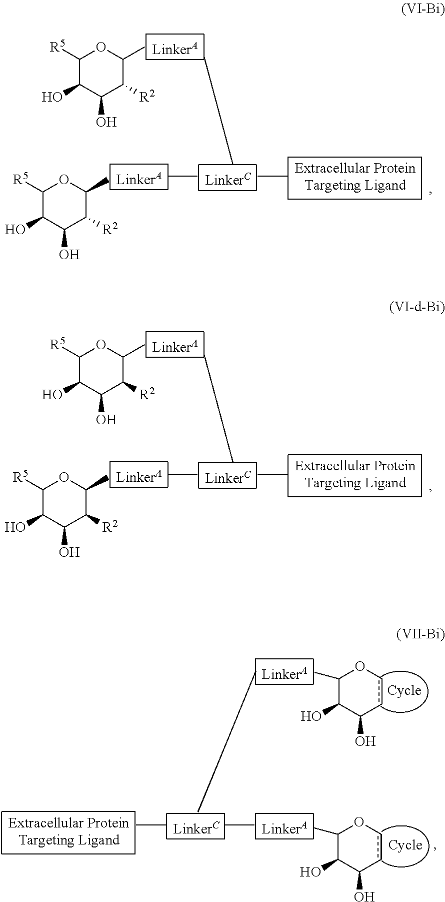 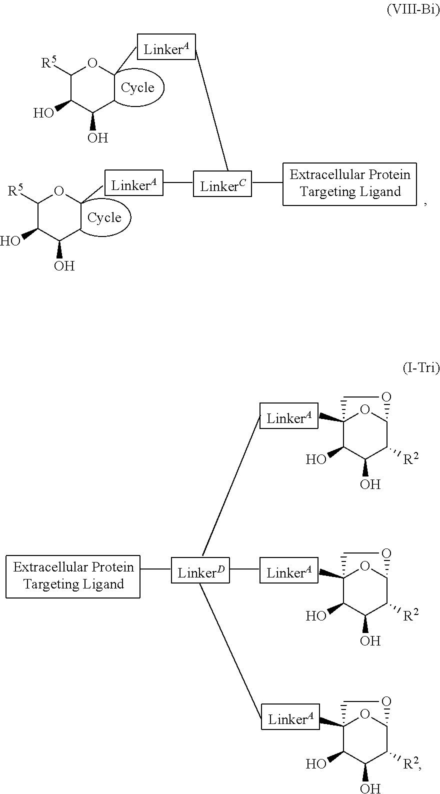 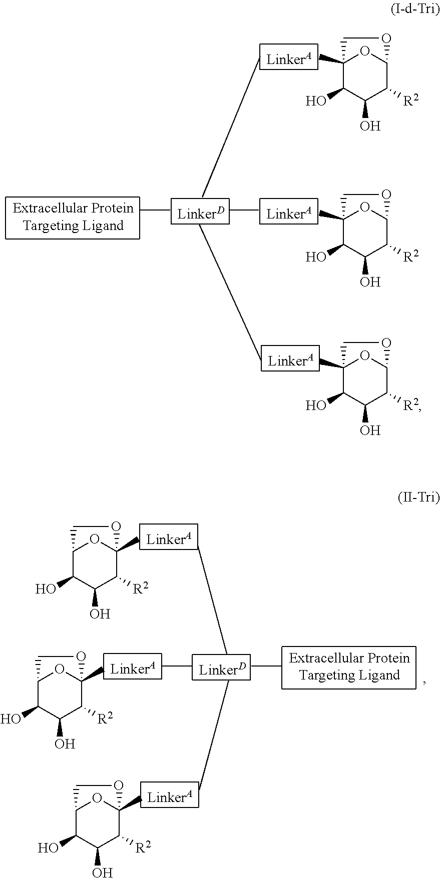 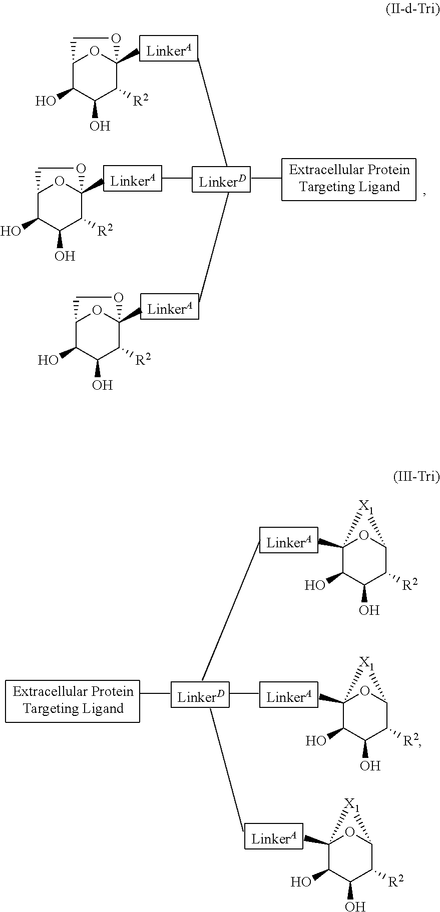 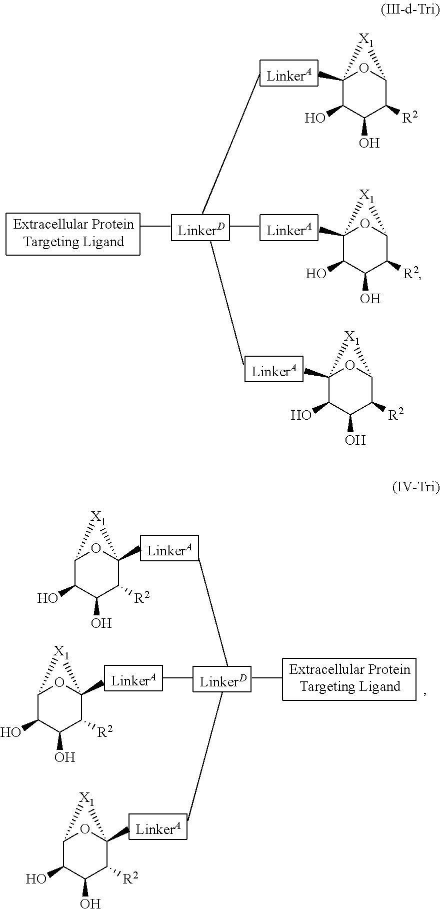 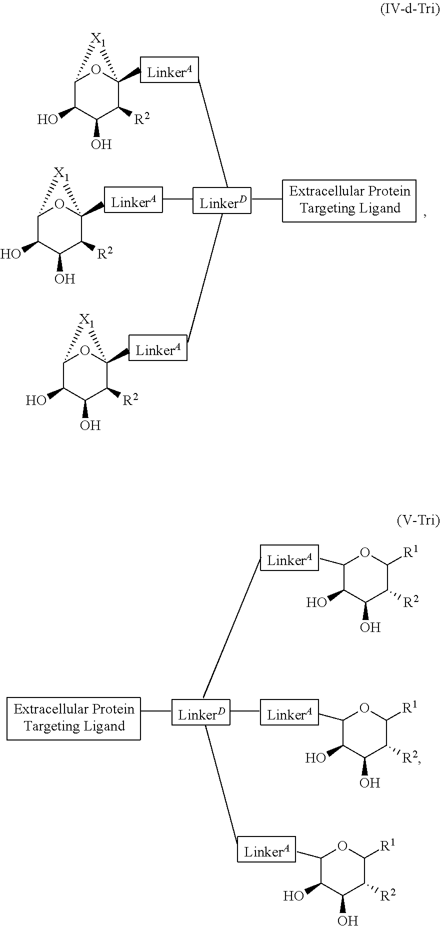 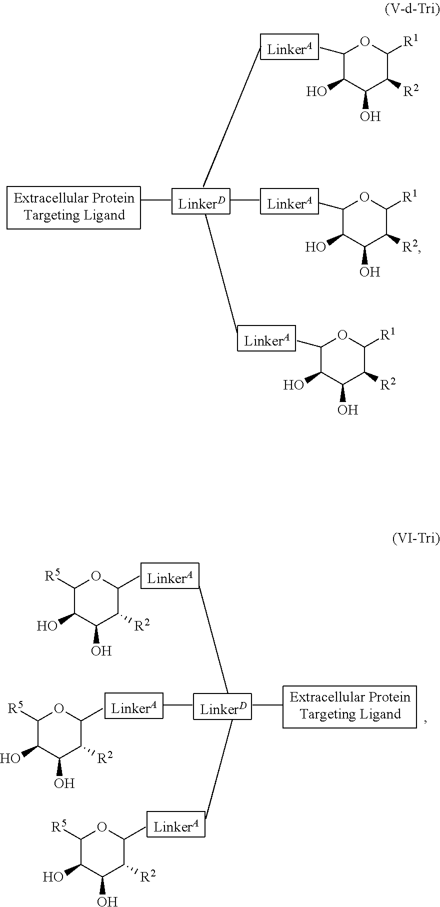 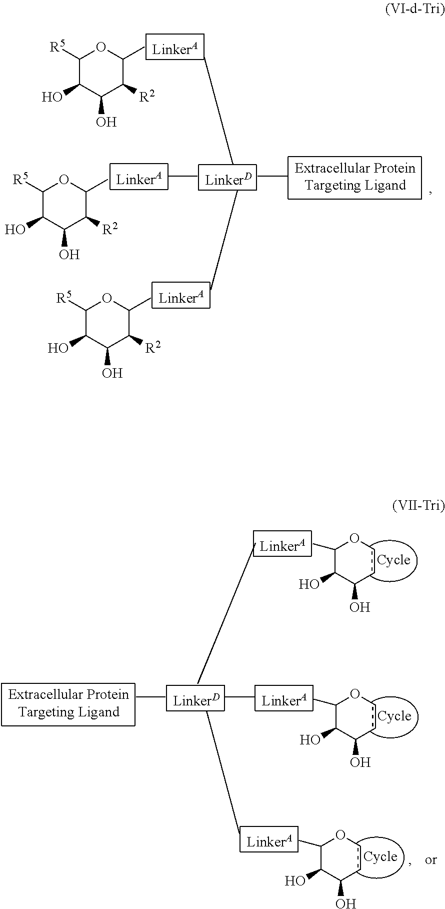 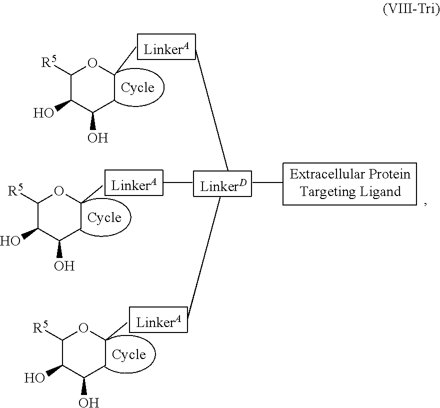
or a pharmaceutically acceptable salt thereof
wherein:
Extracellular Protein Targeting Ligand is a Circulating Protein Binding Moiety [CPBM] which binds to a circulating protein in a subject, wherein the circulating protein mediates a disease state or condition and is to be removed by the action of hepatocytes or other cells of the subject;
X¹ is t to 5 groups independently selected from O, S, N(R⁶), and C(R⁴)(R⁴), wherein if X¹ is 1 group then X¹ is O, S, N(R⁶), or C(R⁴)(R⁴), if X¹ is 2 groups then no more than 1 group of X¹ is, O, S, or N(R⁶), if X¹ is 3, 4, or 5 groups then no more than 2 groups of X¹ are O, S, or N(R⁶)
R² is selected from
(i) aryl, heterocycle, and heteroaryl containing 1 or 2 heteroatoms independently selected from N, O, and S, each of which aryl, heterocycle, and heteroaryl is optionally substituted with 1, 2, 3, or 4 substituents;
(ii) 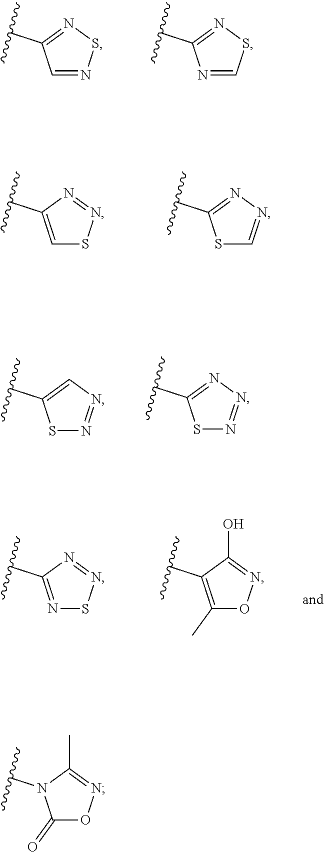
(iii) —NR⁸—S(O)(NR⁶)—R³, —NR⁸—C(S)—R³, —NR⁸—S(O)(R⁶)—R³, —N═S(O)(R³)₂, —NR⁸C(O)NR⁹S(O)₂R³, —NR⁸—S(O)₂—R¹⁰ and —NR⁸—C(NR⁶)—R³ each of which is optic any substituted with 1, 2, 3, or 4 substituents; and
(iv) hydrogen, R¹⁰, alkyl-C(O)—R³, —C(O)—R³, alkyl, haloalkyl, —OC(O)R³, and —NR⁸—C(O)R¹⁰;
R¹⁰ is selected from aryl, alkyl-NR⁸—C(O)—R³, alkyl-aryl, alkyl-heteroaryl with 1, 2, or 4 heteroatoms, alkyl-cyano, alkyl-OR⁶, alkyl-NR⁶R⁸, NR⁸—NR⁶—C(O)R³, NR⁸—S(O)₂—R³, alkenyl, allyl, alkynyl, —NR⁶-alkenyl, —O-alkenyl, —NR⁶-alkynyl, —NR⁶-heteroaryl, —NR⁶-aryl, —O-heteroaryl, —O-aryl and —O-alkynyl, each of which R¹⁰ is optionally substituted with 1, 2, 3, or 4 substituents;
R¹ and R⁵ are independently selected from hydrogen, heteroalkyl, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, —O-alkenyl, —O-alkynyl, C₀-C₆alkyl-OR⁶, C₀-C6alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆akyl-N(R⁸)—S(O)R³, C₀-C₆alkyl-N(R⁸)—C(S)R³, C₀-C6alkyl-N(R⁸)S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents;
R³ at each occurrence is independently selected from hydrogen, alkyl, heteroalkyl, haloalkyl (including —CF₃, —CHF₂, —CH₂F, —CH₂CF₃, —CH₂CH₂F, and —CF₂CF₃), arylalkyl, heteroarylalkyl, alkenyl, alkynyl, aryl, heteroaryl, heterocycle, —OR⁸, and —NR⁸R⁶;
R⁴ is independently selected at each occurrence from hydrogen, heteroalkyl, alkyl, haloalkyl, arylalkyl, heteroarylalkyl, alkenyl, alkynyl, aryl, heteroaryl, heterocycle, -OR⁶, —NR⁶R⁷, C(O)R³, S(O)R³, C(S)R³, and S(O)₂R³,
R⁸ and R⁹ are independently selected at each occurrence from hydrogen, heteroalkyl, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, heteroalkyl, alkyl, arylalkyl, heteroarylalkyl, alkenyl, alkynyl, aryl, heteroaryl, and heterocycle;
Cycle is a 3-8 membered fused cyclic group optionally substituted with 1, 2, 3, or 4 substituents;
each Linker^{A} is a bond or a moiety that covalently links the ASGPR ligand to Linker^{B};
Linker^{B} is a bond or a moiety that covalently links Linker^{A} to an Extracellular Protein Targeting Ligand;
Linker^{C} is a chemical group that links each Linker^{A} to the Extracellular Protein Targeting Ligand; and
Linker^{D} is a chemical group that links each Linker^{A} to the Extracellular Protein Targeting Ligand; and
wherein, when R² is NR⁶-alkenyl, —NR⁶-alkynyl, —NR⁸—C(O)R¹⁰, —NR⁸—S(O)₂-alkenyl, —NR⁸—S(O)₂alkynyl, —NR⁶-heteroaryl, or —NR⁶-aryl, then Extracellular Protein Targeting Ligand does not comprise oligonucleotide; and
the optional substituents are selected from alkyl, alkenyl, alkynyl, haloalkyl, —OR⁶, F, Cl, Br, I, —NR⁶R⁷, heteroalkyl, cyano, nitro, C(O)R³, 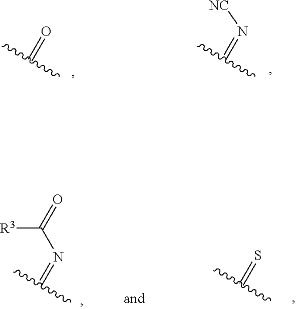
as allowed by valence such that a stable compound results.

29. A method of treating, ameliorating, or preventing a disease or disorder in a subject, the method comprising administering a therapeutically effective amount of at least one compound of claim 1.

30. The method of claim 29, wherein the disease or disorder comprises an autoimmune disease, cancer, or inflammation.

31. The method of claim 30, wherein the autoimmune disease comprises Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (AP1S), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, esosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymysositis, acute disseminated encephalomyelitis (ADEM), acute motor axonic neuropathy, anti-NMDA receptor encephalitis, Balo concentric sclerosis, Bickerstaffs encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, or pyoderma gangrenosum.

32. The method of claim 30, wherein the cancer comprises prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer, or lymphoma.

33. The method of claim 30, wherein the inflammation comprises inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, or arteriosclerosis.

34. The method of claim 29, wherein the subject is further administered at least one additional therapeutic agent that treats, ameliorates, or prevents the disease or disorder.

35. The method of claim 29, wherein the subject is a mammal.

36. The method of claim 29, wherein the subject is a human.

37. A method of treating, preventing, or preventing an autoimmune disease, cancer, or inflammation, the method comprising:
administering a composition comprising a therapeutically effective amount of at least one compound of claim 27 and at least one pharmaceutically acceptable carrier or excipient,
wherein the autoimmune disease comprises Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (AP1S), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, esosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymysositis, acute disseminated encephalomyelitis (ADEM), acute motor axonic neuropathy, anti-NMDA receptor encephalitis, Balo concentric sclerosis, Bickerstaffs encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, or pyoderma gangrenosum;
wherein the cancer comprises prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer, or lymphoma; and
wherein the inflammation comprises inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, or arteriosclerosis.
